(19) United States
(12) Patent Application Publication
(10) Pub. No.: US 2020/0263206 A1
(43) Pub. Date: Aug. 20, 2020

(54) TARGETED INTEGRATION SYSTEMS AND METHODS FOR THE TREATMENT OF HEMOGLOBINOPATHIES

(71) Applicant: EDITAS MEDICINE, INC., Cambridge, MA (US)

(72) Inventors: Jennifer Leah GORI, Jamaica Plain, MA (US); Cecilia COTTA-RAMUSINO, Cambridge, MA (US); Carrie M. MARGULIES, Waban, MA (US)

(73) Assignee: EDITAS MEDICINE, INC., Cambridge, MA (US)

(21) Appl. No.: 16/762,360

(22) PCT Filed: Nov. 7, 2018
(86) PCT No.: PCT/US18/59700

§ 371 Date: May 7, 2020

Related U.S. Application Data
(60) Provisional application No. 62/582,905, filed on Nov. 7, 2017.

(51) Int. Cl. C12N 15/90; C12N 15/113; C12N 9/22

(52) U.S. Cl. CPC C12N 15/907; C12N 15/113; C12N 2800/80; C12N 2310/20; C12N 9/22

(57) ABSTRACT

Genome editing systems, guide RNAs, DNA donor templates, and CRISPR-mediated methods are provided for altering a β-globin gene to alter a genotype, e.g., by correcting or partially correcting, a genotype associated with thalassemia or sickle cell disease.

### PRIORITY CLAIM

The present application claims the benefit of U.S. Provisional Application No. 62/582,905, filed Nov. 7, 2017, the contents of which are hereby incorporated by reference in their entirety.

### SEQUENCE LISTING

This application contains a Sequence Listing, which was submitted in ASCII format via EFS-Web, and is hereby incorporated by reference in its entirety. The ASCII copy, created on Nov. 7, 2018, is named SequenceListing.txt and is 480 kilobytes in size.

### FIELD

This disclosure relates to genome editing systems and methods for altering a target nucleic acid sequence, or modulating expression of a target nucleic acid sequence, and applications thereof in connection with the alteration of genes encoding hemoglobin subunits and/or treatment of hemoglobinopathies.

### BACKGROUND

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin (Thein 2009). The α-hemoglobin gene is located on chromosome 16, while the β-hemoglobin gene (HBB), A gamma (γA)-globin chain (HBG1, also known as gamma globin A), and G gamma (γG)-globin chain (HBG2, also known as gamma globin G) are located on chromosome 11 within the globin gene cluster (also referred to as the globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c. 17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA2, an HbA variant in which the two γ-globin chains are replaced with two delta (Δ)-globin chains. δ-Thal is associated with mutations in the Δ hemoglobin gene (HBD) that cause a loss of HBD expression. Co-inheritance of the HBD mutation can mask a diagnosis of β-Thal (i.e., β/δ-Thal) by decreasing the level of HbA2 to the normal range (Bouva 2006). β/δ-Thal is usually caused by deletion of the HBB and HBD sequences in both alleles. In homozygous (δo/δo βo/βo) patients, HBG is expressed, leading to production of HbF alone.

Like SCD, β-Thal is caused by mutations in the HBB gene. The most common HBB mutations leading to β-Thal are: c.-136C>G, c.92+1G>A, c.92+6T>C, c.93-21G>A, c.118C>T, c.316-106C>G, c.25_26delAA, c.27_28insG, c.92+5G>C, c.118C>T, c.135delC, c.315+1G>A, c.-78A>G, c.52A>T, c.59A>G, c.92+5G>C, c.124_127delTTCT, c.316-197C>T, c.-78A>G, c.52A>T, c.124_127delTTCT, c.316-197C>T, c.-138C>T, c.-79A>G, c.92+5G>C, c.75T>A, c.316-2A>G. and c.316-2A>C. These and other mutations associated with β-Thal cause mutated or absent β-globin chains, which causes a disruption of the normal Hb α-hemoglobin to β-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted β⁰/β⁰). β-Thal major results in severe reduction in β-globin chains, leading to significant precipitation of α-globin chains in RBCs and more severe anemia.

β-Thal intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. So represents absent expression of a β-globin chain; β+ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for β-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogeneic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Thus, there is a need for improved methods of managing these and other hemoglobinopathies.

### SUMMARY

Provided herein are genome editing systems, guide RNAs (gRNAs), DNA donor templates, and CRISPR-mediated methods for altering a β-globin gene (e.g., HBB) to alter a genotype, e.g., by correcting, or partially correcting, a genotype associated with thalassemia or SCD.

The compositions and methods described herein allow for the quantitative analysis of on-target gene editing outcomes, including targeted integration events, by embedding one or more primer binding sites (i.e., priming sites) into a donor template that are substantially identical to a priming site present at the targeted genomic DNA locus (such as at least one allele of the HBB gene, which is referred to interchangeably herein as the “target nucleic acid”). The priming sites are embedded into the donor template such that, when homologous recombination of the donor template with at least one allele of the HBB gene occurs, successful targeted integration of the donor template integrates the priming sites from the donor template into the target nucleic acid such that at least one amplicon can be generated in order to quantitatively determine the on-target editing outcomes.

In some embodiments, the at least one allele of the HBB gene comprises a first priming site (P1) and a second priming site (P2), and the donor template comprises a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to P1, and wherein P2′ is substantially identical to P2. After accurate homology-driven targeted integration, three amplicons are produced using a single PCR reaction with two oligonucleotide primers (FIG. 1A). The first amplicon, Amplicon X, is generated from the primer binding sites originally present in the genomic DNA (P1 and P2), and may be sequenced to analyze on-target editing events that do not result in targeted integration (e.g., insertions, deletions, gene conversion). The remaining two amplicons are mapped to the 5′ and 3′ junctions after homology-driven targeted integration. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the at least one allele of the HBB gene, thereby amplifying the 3′ junction. Sequencing of these amplicons provides a quantitative assessment of targeted integration at the at least one allele of the HBB gene, in addition to information about the fidelity of the targeted integration. To avoid any biases inherent to amplicon size, stuffer sequences may optionally be included in the donor template to keep all three expected amplicons the same length.

In one aspect, disclosed herein is a genome editing system, comprising:

a ribonucleic acid (RNA) guided nuclease:

a guide RNA targeting a target nucleic acid of an HBB gene; and

an isolated nucleic acid for integration into the HBB gene, wherein:

(a) a first strand of the target nucleic acid comprises, from 5′ to 3′. P1--H1--X--H2--P2, wherein

P1 is a first priming site;

H1 is a first homology arm;

X is the cleavage site:

H2 is a second homology arm; and

P2 is a second priming site; and
(b) a first strand of the isolated nucleic acid comprises, from 5′ to 3′, A1--P2′-N--A2, or
   A1--N--P1′--A2, wherein

A1 is a homology arm that is substantially identical to H1;

P2′ is a priming site that is substantially identical to P2;

N is a cargo;

P1′ is a priming site that is substantially identical to P1; and

A2 is a homology arm that is substantially identical to H2.

In one aspect, disclosed herein is an isolated nucleic acid for homologous recombination with at least one allele of the HBB gene having a cleavage site, wherein:

(a) a first strand of the at least one allele of the HBB gene comprises, from 5′ to 3′, P1--H1--X--H2--P2, wherein

P1 is a first priming site;

H1 is a first homology arm:

X is the cleavage site;

H2 is a second homology arm; and

P2 is a second priming site; and

(b) a first strand of the isolated nucleic acid comprises, from 5′ to 3′, A1--P2′-N--A2, or

A1--N--P1′--A2, wherein

A1 is a homology arm that is substantially identical to H1;

P2′ is a priming site that is substantially identical to P2;

N is a cargo;

P1′ is a priming site that is substantially identical to P1; and

A2 is a homology arm that is substantially identical to H2.

In one embodiment, the first strand of the isolated nucleic acid comprises, from 5′ to 3′, A1-P2′--N--P1′--A2. In one embodiment, the first strand of the isolated nucleic acid further comprises S1 or S2, wherein the first strand of the isolated nucleic acid comprises, from 5′ to 3′,

### A1--S1-P2′-N--A2, or A1--N--P1′-S2-A2;

wherein S1 is a first stuffer, wherein S2 is a second stuffer, and wherein each of S1 and S2 comprise a random or heterologous sequence having a GC content of approximately 40%6.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the isolated nucleic acid comprises, from 5′ to 3′, A1-S1--P2′-N-P1′--S2--A2. In one embodiment, A1+S1 and A2+S2 have sequences that are of approximately equal length. In one embodiment, A1+S1 and A2+S2 have sequences that are of equal length. In one embodiment, A1+S1 and H1+X+H2 have sequences that are of approximately equal length. In one embodiment, A1+S1 and H1+X+H2 have sequences that are of equal length. In one embodiment, A2+S2 and H1+X+H2 have sequences that are of approximately equal length. In one embodiment, A2+S2 and H1+X+H2 have sequences that are of equal length.

In one embodiment, A1 has a sequence that is at least 40 nucleotides in length, and A2 has a sequence that is at least 40 nucleotides in length.

In one embodiment, A1 has a sequence that is identical to, or differs by no more than 1, 2, 3, 4, 5, 6, 7, 8, 9, 10, 15, 20, 25, or 30 nucleotides from a sequence of H1. In one embodiment. A2 has a sequence that is identical to, or differs by no more than 1, 2, 3, 4, 5, 6, 7, 8, 9, 10, 15, 20, 25, or 30 nucleotides from a sequence of H2.

In one embodiment, A1+S1 have a sequence that is at least 40 nucleotides in length, and A2+S2 have a sequence that is at least 40 nucleotides in length.

In one embodiment, N comprises an exon of a gene sequence, an intron of a gene sequence, a cDNA sequence, or a transcriptional regulatory element; a reverse complement of any of the foregoing or a portion of any of the foregoing. In one embodiment. N comprises a promoter sequence.

In one aspect, disclosed herein is a composition comprising an isolated nucleic acid disclosed herein and, optionally, a pharmaceutically acceptable carrier.

In one aspect, disclosed herein is a vector comprising an isolated nucleic acid disclosed herein. In one embodiment, the vector is a viral vector. In one embodiment, the vector is an AAV vector, a lentivirus, a naked DNA vector, or a lipid nanoparticle.

In one aspect, disclosed herein is a genome editing system comprising an isolated nucleic acid disclosed herein. In one embodiment, the genome editing system further comprises a RNA-guided nuclease and at least one gRNA molecule.

In one aspect, disclosed herein is a method of altering a cell comprising contacting the cell with a genome editing system.

In one aspect, disclosed herein is a kit comprising a genome editing system.

In one aspect, disclosed herein is a nucleic acid, composition, vector, gene editing system, method or kit, for use in medicine.

In one aspect, disclosed herein is a method of altering a cell, comprising the steps of: forming, in at least one allele of the HBB gene of the cell, at least one single- or double-strand break at a cleavage site, wherein the at least one allele of the HBB gene comprises a first strand comprising: a first homology arm 5′ to the cleavage site, a first priming site either within the first homology arm or 5′ to the first homology arm, a second homology arm 3′ to the cleavage site, and a second priming site either within the second homology arm or 3′ to the second homology arm, and recombining an exogenous oligonucleotide donor template with the at least one allele of the HBB gene by homologous recombination to produce an altered nucleic acid, wherein a first strand of the exogenous oligonucleotide donor template comprises either: i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site either within or 3′ to the cargo, and a second donor homology arm 3′ to the cargo, thereby altering the cell.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm. In one embodiment, the first strand of the exogenous oligonucleotide donor template further comprises a first stuffer or a second stuffer, wherein the first stuffer and the second stuffer each comprise a random or heterologous sequence having a GC content of approximately 40%; and wherein the first strand of the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, the step of forming the at least one single- or double-strand break comprises contacting the cell with an RNA-guided nuclease. In one embodiment, the RNA-guided nuclease is a Class 2 Clustered Regularly Interspersed Repeat (CRISPR)-associated nuclease. In one embodiment, the RNA-guided nuclease is selected from the group consisting of wild-type Cas9, a Cas9 nickase, a wild-type Cpf1, and a Cpf1 nickase.

In one embodiment, the step of contacting the RNA-guided nuclease with the cell comprises introducing into the cell a ribonucleoprotein (RNP) complex comprising the RNA-guided nuclease and a guide RNA (gRNA). In one embodiment, the step of recombining the exogenous oligonucleotide donor template into the nucleic acid by homologous recombination comprises introducing the exogenous oligonucleotide donor template into the cell.

In one embodiment, the step of introducing comprises electroporation of the cell in the presence of the RNP complex and/or the exogenous oligonucleotide donor template.

In one aspect, disclosed herein is a method of altering at least one allele of the HBB gene in a cell, wherein the at least one allele of the HBB gene comprises a first strand comprising: a first homology arm 5′ to a cleavage site, a first priming site either within the first homology arm or 5′ to the first homology arm, a second homology arm 3′ to the cleavage site, and a second priming site either within the second homology arm or 3′ to the second homology arm, the method comprising: contacting the cell with (a) at least one gRNA molecule, (b) a RNA-guided nuclease molecule, and (c) an exogenous oligonucleotide donor template, wherein a first strand of the exogenous oligonucleotide donor template comprises either: i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site, and a second donor homology arm 3′ to the cargo; wherein the gRNA molecule and the RNA-guided nuclease molecule interact with the at least one allele of the HBB gene, resulting in a cleavage event at or near the cleavage site, and wherein the cleavage event is repaired by at least one DNA repair pathway to produce an altered nucleic acid, thereby altering the at least one allele of the HBB gene in the cell.

In one embodiment, the method further comprises contacting the cell with (d) a second gRNA molecule, wherein the second gRNA molecule and the RNA-guided nuclease molecule interact with the at least one allele of the HBB gene, resulting in a second cleavage event at or near the cleavage site, and wherein the second cleavage event is repaired by the at least one DNA repair pathway.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

In one embodiment, the first strand of the exogenous oligonucleotide donor template further comprises a first stuffer or a second stuffer, wherein the first stuffer and the second stuffer each comprise a random or heterologous sequence having a GC content of approximately 40%; and wherein the first strand of the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, the cell is contacted first with the at least one gRNA molecule and the RNA-guided nuclease molecule, followed by contacting the cell with the exogenous oligonucleotide donor template. In one embodiment, the cell is contacted with the at least one gRNA molecule, the RNA-guided nuclease molecule, and the exogenous oligonucleotide donor template at the same time.

In one embodiment, the exogenous oligonucleotide donor template is present in a vector. In one embodiment, the vector is a viral vector. In one embodiment, the viral vector is an AAV vector or a lentiviral vector.

In one embodiment, the DNA repair pathway repairs the target nucleic acid to result in targeted integration of the exogenous oligonucleotide donor template. In one embodiment, the altered nucleic acid comprises a sequence comprising an indel as compared to a sequence of the target nucleic acid. In one embodiment, the cleavage event, or both the cleavage event and the second cleavage event, is/are repaired by gene correction.

In one embodiment, the first donor homology arm and the first stuffer consist of a sequence that is of approximately equal length to a sequence consisting of the second donor homology arm and the second stuffer. In one embodiment, the first donor homology arm and the first stuffer consist of a sequence that is of equal length to the sequence consisting of the second donor homology arm and the second stuffer.

In one embodiment, the first donor homology arm and the first stuffer consist of a sequence that is of approximately equal length to a sequence consisting of the first homology arm, the cleavage site, and the second homology arm. In one embodiment, the first donor homology arm and the first stuffer consist of a sequence that is of equal length to a sequence consisting of the first homology arm, the cleavage site, and the second homology arm.

In one embodiment, the second donor homology arm and the second stuffer consist of a sequence that is of approximately equal length to a sequence consisting of the first homology arm, the cleavage site, and the second homology arm. In one embodiment, the second donor homology arm and the second stuffer consist of a sequence that is of equal length to a sequence consisting of the first homology arm, the cleavage site, and the second homology arm.

In one embodiment, the first donor homology arm has a sequence that is at least 40 nucleotides in length, and wherein the second donor homology arm has a sequence that is at least 40 nucleotides in length. In one embodiment, the first donor homology arm has a sequence that is identical to, or differs by no more than 1, 2, 3, 4, 5, 6, 7, 8, 9, 10, 15, 20, 25, or 30 nucleotides from, a sequence of the first homology arm. In one embodiment, the second donor homology arm has a sequence that is identical to, or differs by no more than 1, 2, 3, 4, 5, 6, 7, 8, 9, 10, 15, 20, 25, or 30 nucleotides from, a sequence of the second homology arm.

In one embodiment, the first donor homology arm and the first stuffer consist of a sequence that is at least 40 nucleotides in length, and the second donor homology arm and the second stuffer consist of a sequence that is at least 40 nucleotides in length.

In one embodiment, the first suffer has a sequence that is different from a sequence of the second stuffer.

In one embodiment, the first priming site, the priming site that is substantially identical to the first priming site, the second priming site, and the priming site that is substantially identical to the second priming site are each less than 60 base pairs in length.

In one embodiment, the method further comprises amplifying the target nucleic acid, or a portion of the target nucleic acid, prior to the forming step or the contacting step.

In one embodiment, the method further comprises amplifying the altered nucleic acid using a first primer which binds to the first priming site and/or the priming site that is substantially identical to the first priming site, and a second primer which binds to the second priming site and/or the priming site that is substantially identical to the second priming site.

In one embodiment, the altered nucleic acid comprises a sequence that is different than a sequence of the target nucleic acid.

In one embodiment, the gRNA molecule is a gRNA nucleic acid, and wherein the RNA-guided nuclease molecule is a RNA-guided nuclease protein. In one embodiment, the gRNA molecule is a gRNA nucleic acid, and wherein the RNA-guided nuclease molecule is a RNA-guided nuclease nucleic acid. In one embodiment, the cell is contacted with the gRNA molecule and the RNA-guided nuclease molecule as a pre-formed complex. In one embodiment, the RNA-guided nuclease is selected from the group consisting of wild-type Cas9, a Cas9 nickase, a wild-type Cpf1, and a Cpf1 nickase.

In one embodiment, the target nucleic acid comprises an exon of a gene, an intron of a gene, a cDNA sequence, a transcriptional regulatory element: a reverse complement of any of the foregoing; or a portion of any of the foregoing.

In one embodiment, the cell is a eukaryotic cell. In one embodiment, the eukaryotic cell is a human cell.

In one embodiment, the cell is from a subject suffering from a disease or disorder. In one embodiment, the disease or disorder is a blood disease, an immune disease, a neurological disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or a pain disorder.

In one embodiment, the cell is from a subject having at least one mutation at the cleavage site.

In one embodiment, the method further comprises isolating the cell from the subject prior to contacting the forming step or the contacting step.

In one embodiment, the method further comprises introducing the cell into a subject after the recombining step or after the cleavage event is repaired by the at least one DNA repair pathway.

In one embodiment, the forming step and the recombining step, or the contacting step, is performed in vitro. In one embodiment, the forming step and the recombining step, or the contacting step, is performed ex vivo. In one embodiment, the forming step and the recombining step, or the contacting step, is performed in vivo.

In one aspect, disclosed herein is a method for determining the outcome of a gene editing event at a cleavage site in a target nucleic acid in a cell using an exogenous donor template, wherein the target nucleic acid comprises a first strand comprising: a first homology arm 5′ to a cleavage site, a first priming site either within the first homology arm or 5′ to the first homology arm, a second homology arm 3′ to the cleavage site, and a second priming site either within the second homology arm or 3′ to the second homology arm, and wherein a first strand of the exogenous donor template comprises i) a cargo, a priming site that is substantially identical to the second priming site either within or 5′ to the cargo, a first donor homology arm 5′ to the cargo, and a second donor homology arm 3′ to the cargo; or ii) a cargo, a first donor homology arm 5′ to the cargo, a priming site that is substantially identical to the first priming site 3′ to the cargo, and a second donor homology arm 3′ to the cargo, the method comprising: i) forming at least one single- or double-strand break at or near the cleavage site in the target nucleic acid; ii) recombining the exogenous oligonucleotide donor template with the target nucleic acid via homologous recombination to produce an altered nucleic acid; and iii) amplifying the altered nucleic acid using a first primer which binds to the first priming site and/or the priming site that is substantially identical to the first priming site; and/or a second primer which binds to the second priming site and/or the priming site that is substantially identical to the second priming site: thereby determining the outcome of the gene editing event in the cell.

In one embodiment, the step of forming the at least one single- or double-strand break comprises contacting the cell with an RNA-guided nuclease. In one embodiment, the RNA-guided nuclease is a Class 2 Clustered Regularly Interspersed Repeat (CRISPR)-associated nuclease. In one embodiment, the RNA-guided nuclease is selected from the group consisting of wild-type Cas9, a Cas9 nickase, a wild-type Cpf1, and a Cpf1 nickase.

In one embodiment, the step of contacting the RNA-guided nuclease with the cell comprises introducing into the cell a ribonucleoprotein (RNP) complex comprising the RNA-guided nuclease and at least one guide RNA (gRNA). In one embodiment, the step of recombining the exogenous oligonucleotide donor template into the nucleic acid via homologous recombination comprises introducing the exogenous oligonucleotide donor template into the cell. In one embodiment, the step of introducing comprises electroporation of the cell in the presence of the RNP complex and/or the exogenous oligonucleotide donor template.

In one embodiment, the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

In one embodiment, the first strand of the exogenous oligonucleotide donor template further comprises a first stuffer and/or a second stuffer, wherein the first stuffer and the second stuffer each comprise a random or heterologous sequence having a GC content of approximately 40%; and wherein the exogenous oligonucleotide donor template comprises, from 5′ to 3′, i) the first donor homology arm, the first stuffer, the priming site that is substantially identical to the second priming site, and the second donor homology arm; or ii) the first donor homology arm, the cargo, the priming site that is substantially identical to the first priming site, the second stuffer, and the second donor homology arm.

In one embodiment, the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site. In one embodiment, the first stuffer has a sequence comprising at least 10 nucleotides of a sequence set forth in Table 2, and wherein the second stuffer has a sequence comprising at least 10 nucleotides of a sequence set forth in Table 2. In one embodiment, the first stuffer has a sequence that is not the same as the sequence of the second stuffer.

In one embodiment, the first strand of the exogenous oligonucleotide donor template comprises, from 5′ to 3′, the first donor homology arm, the first suffer, the priming site that is substantially identical to the second priming site, the cargo, the priming site that is substantially identical to the first priming site, the second stuffer, and the second donor homology arm.

In one embodiment, the altered nucleic acid comprises, from 5′ to 3′, the first priming site, the first donor homology arm, the priming site that is substantially identical to the second priming site, the cargo, the second donor homology arm, and the second priming site. In one embodiment, the altered nucleic acid comprises, from 5′ to 3′, the first priming site, the first donor homology arm, the cargo, the priming site that is substantially identical to the first priming site, the second donor homology arm, and the second priming site.

In one embodiment, the altered nucleic acid comprises, from 5′ to 3′, the first priming site, the first donor homology arm, the priming site that is substantially identical to the second priming site, the cargo, the priming site that is substantially identical to the first priming site, the second donor homology arm, and the second priming site.

In one embodiment, the altered nucleic acid comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

In one embodiment, when the altered nucleic acid comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

In one embodiment, when the altered nucleic acid comprises a targeted integration genome editing event at the cleavage site, amplifying the altered nucleic acid using the first primer and the second primer produces a first amplicon, wherein the first amplicon has a sequence that is substantially identical to a sequence consisting of either i) the first donor homology arm and the first stuffer, or ii) the second stuffer and the second donor homology arm.

In one embodiment, when the altered nucleic acid comprises a targeted integration genome editing event at the cleavage site, amplifying the altered nucleic acid using the first primer and the second primer produces a first amplicon and a second amplicon, wherein the first amplicon has a sequence that is substantially identical to a sequence consisting of the first donor homology arm and the first stuffer, and wherein the second amplicon has a sequence that is substantially identical to a sequence consisting of the second stuffer and the second homology arm.

In one embodiment, the cell is a population of cells, and when the altered nucleic acid in all cells within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid.

In one embodiment, the cell is a population of cells, and when the altered nucleic acid in all the cells within the population of cells comprises a targeted integration genome editing event at the cleavage site, amplifying the altered nucleic acid using the first primer and the second primer produces a first amplicon, wherein the first amplicon has a sequence that is substantially identical to a sequence consisting of either i) the first donor homology arm and the first stuffer, or ii) the second stuffer and the second donor homology arm.

In one embodiment, the cell is a population of cells, and when the altered nucleic acid in a first cell within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid; and when the altered nucleic acid in a second cell within the population of cells comprises a targeted integration genome editing event at the cleavage site, amplifying the altered nucleic acid in the second cell using the first primer and the second primer produces a second amplicon, wherein the second amplicon has a sequence that is substantially identical to a sequence consisting of either i) the first donor homology arm and the first stuffer, or ii) the second stuffer and the second donor homology arm.

In one embodiment, the cell is a population of cells, when the altered nucleic acid in a first cell within the population of cells comprises a non-targeted integration genome editing event at the cleavage site, amplifying the altered nucleic acid using the first primer and the second primer produces a first amplicon, wherein the first amplicon has a sequence that comprises an indel as compared to a sequence of the target nucleic acid; and when the altered nucleic acid in a second cell within the population of cells comprises a targeted integration genome editing event at the cleavage site, amplifying the altered nucleic acid in the second cell using the first primer and the second primer produces a second amplicon and a third amplicon, wherein the second amplicon has a sequence that is substantially identical to a sequence consisting of the first donor homology arm and the first stuffer, and wherein the third amplicon has a sequence that is substantially identical to a sequence consisting of the second stuffer and the second donor homology arm.

In one embodiment, frequency of targeted integration versus non-targeted integration in the population of cells can be measured by: i) the ratio of ((an average of the second amplicon plus the third amplicon)/(first amplicon plus (the average of the second amplicon plus the third amplicon)); ii) the ratio of (the second amplicon/(the first amplicon plus the second amplicon)); or iii) the ratio of (the third amplicon/(the first amplicon plus the third amplicon)).

In one aspect, disclosed herein is a cell, or a population of cells, altered by a method disclosed herein.

This listing is intended to be exemplary and illustrative rather than comprehensive and limiting. Additional aspects and embodiments may be set out in, or apparent from, the remainder of this disclosure and the claims.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1A is a schematic representation of an unedited genomic DNA targeting site, an exemplary DNA donor template for targeted integration, potential insertion outcomes (i.e., non-targeted integration at the cleavage site or targeted integration at the cleavage site) and three potential PCR amplicons resulting from use of a primer pair targeting the P1 priming site and the P2 primer site (Amplicon X), a primer pair targeting the P1 primer site and the P2′ priming site (Amplicon Y), or a primer pair targeting the P1′ primer site and the P2 primer site (Amplicon Z). The depicted exemplary DNA donor template contains integrated primer sites (P1′ and P2′) and stuffer sequences (S1 and S2). A1/A2: donor homology arms, S1/S2: donor stuffer sequences, P1/P2: genomic primer sites, P1′/P2′: integrated primer sites, H1/H2: genomic homology arms, N: cargo, X: cleavage site.

FIG. 1B is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the P primer site and the P2 primer site (Amplicon X), or a primer pair targeting the P1′ primer site and the P2 primer site (Amplicon Y). The exemplary DNA donor template contains an integrated primer site (P1′) and a stuffer sequence (S2). A1/A2: donor homology arms, S1/S2: donor stuffer sequences, P1/P2: genomic primer sites, P1′: integrated primer sites, H1/H2: genomic homology arms, N: cargo, X: cleavage site.

FIG. 1C is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the P primer site and the P2 primer site (Amplicon X), or a primer pair targeting the P1 primer site and the P2′ primer site (Amplicon Y). The exemplary DNA donor template contains an integrated primer site (P2′) and a stuffer sequence (S1). A1/A2: donor homology arms, S1/S2: donor stuffer sequences, P1/P2: genomic primer sites, P2′: integrated primer sites, H1/H2: genomic homology arms, N: cargo, X: cleavage site.

FIG. 2A depicts exemplary DNA donor templates comprising either long homology arms (“500 bp HA”), short homology arms (“177 bp HA”), or no homology arms (“No HA”) used for targeted integration experiments in primary CD4+ T-cells using wild-type S. pyogenes ribonucleoprotein targeted to the HBB locus. FIGS. 2B, 2C and 2D depict that DNA donor templates with either long homology arms and short homology arms have similar targeted integration efficiency in CD4+ T-cells as measured using GFP expression and ddPCR (5′ and 3′ junctions). FIG. 2B shows the GFP fluorescence of CD4+ T-cells contacted with wild-type S. pyogenes ribonucleoprotein and one of the DNA donor templates depicted in FIG. 2A at different multiplicities of infection (MOI). FIGS. 2C and 2D shows the integration frequency in CD4+ T cells contacted with wild-type S. pyogenes ribonucleoprotein (RNP) and one of the DNA donor templates depicted in FIG. 2A at different multiplicities of infection (MOI), as determined using ddPCR amplifying the 5′ integration junction (FIG. 2C) or the 3′ integration junction (FIG. 2D).

FIG. 3 depicts the quantitative assessment of on-target editing events from sequencing at HBB locus as determined using Sanger sequencing.

FIG. 4 depicts the experimental schematic for evaluation of HDR and targeted integration in CD34+ cells.

FIGS. 5A-B depict the on-target integration as detected by ddPCR analysis of (FIG. SA) the 5′ and (FIG. 5B) the 3′ vector-genomic DNA junctions on day 7 in gDNA from CD34+ cells that were untreated (−) or treated with RNP+ AAV6+/−homology arms (HA). FIG. 5C Depicts % GFP⁺ cells detected on day 7 in the live CD34⁺ cell fraction which shows that the integrated transgene is expressed from a genomic context.

FIG. 6 depicts the DNA sequencing results for the cells treated with RNP+ AAV6+/−HA with % gene modification comprised of HDR (targeted integration events and gene conversion) and NHEJ (Insertions, Deletions, Insertions from AAV6 donor).

FIG. 7 depicts the kinetics of CD34+ cell viability up to 7 days after treatment with electroporation alone (EP control), or electroporation with RNP or RNP+ AAV6. Viability was measured by Acridine Orange/Propidium Iodide (AOPI).

FIG. 8 depicts flow cytometry results which show GFP expression in erythroid and myeloid progeny of edited cells. The boxed gate calls out the events that were positive for erythroid (CD235) or myeloid (CD33) surface antigen (quadrant gates). GFP+ events were scored within the myeloid and erythroid cell populations (boxed gates).

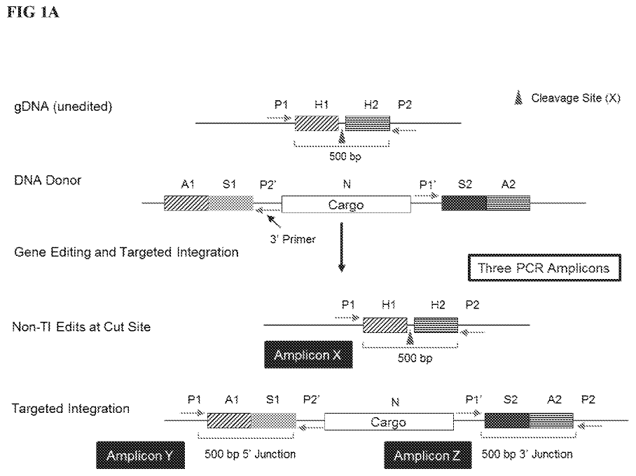

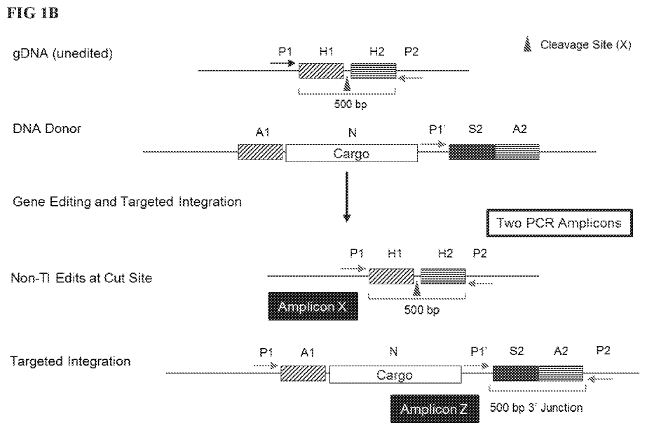

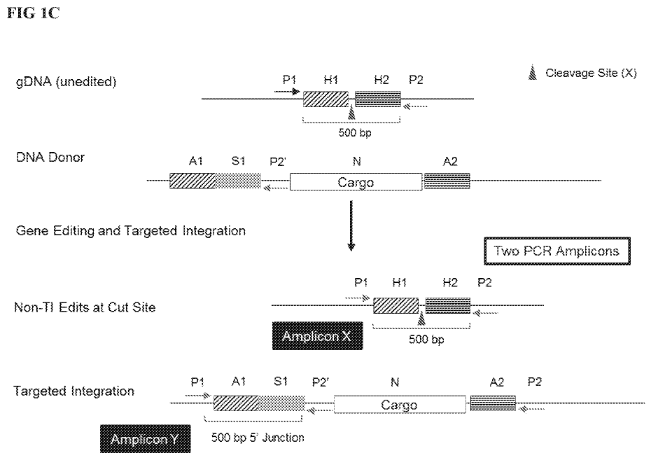

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Domain” is used to describe a segment of a protein or nucleic acid. Unless otherwise indicated, a domain is not required to have any specific functional property.

The term “exogenous trans-acting factor” refers to any peptide or nucleotide component of a genome editing system that both (a) interacts with an RNA-guided nuclease or gRNA by means of a modification, such as a peptide or nucleotide insertion or fusion, to the RNA-guided nuclease or gRNA, and (b) interacts with a target DNA to alter a helical structure thereof. Peptide or nucleotide insertions or fusions may) include, without limitation, direct covalent linkages between the RNA-guided nuclease or gRNA and the exogenous trans-acting factor, and/or non-covalent linkages mediated by the insertion or fusion of RNA/protein interaction domains such as MS2 loops and protein/protein interaction domains such as a PDZ, Lim or SHI, 2 or 3 domains. Other specific RNA and amino acid interaction motifs will be familiar to those of skill in the art. Trans-acting factors may include, generally, transcriptional activators.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g., a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai 2016 (incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid. e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid. e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g., a nucleic acid or protein), does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state: relieving one or more symptoms of the disease; and curing the disease.

“Prevent,” “preventing,” and “prevention” refer to the prevention of a disease in a subject, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a gRNA complexed or able to complex with an RNA-guided nuclease, and accompanied by (e.g., suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Comish-Bowden 1985, incorporated by reference herein). It should be noted, however, that “T” denotes “Thymine or Uracil” in those instances where a sequence may be encoded by either DNA or RNA for example in gRNA targeting domains.

TABLE 1
IUPAC nucleic acid notation

| Character | Base |
|---|---|
| A | Adenine |
| T | Thymine |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

### Overview

Aspects of this disclosure generally relate to genome editing systems configured to introduce alterations (e.g., one or more deletions, insertions, or other changes) into chromosomal DNA to correct mutations in the HBB gene. Alterations may be made at or proximate to (e.g. within 10, 20, 30, 40, 50, 60, 70, 80, 90 100, 150, 200, 250, 300, 500, 1000 bp of) a site of a mutation associated with SCD (the c.17A>T HbS mutation) or β-thal (including, without limitation c.-136C>G, c.92+1G>A, c.92+6T>C, c.93-21G>A, c.118C>T, c.316-106C>G, c.25_26delAA, c.27_28insG, c.92+5G>C, c.118C>T, c.135delC, c.315+1G>A, c.-78A>G, c.52A>T, c.59A>G, c.92+5G>C, c.124_127delTTCT, c.316-197C>T, c.-78A>G, c.52A>T, c.124_127delTTCT, c.316-197C>T, c.-138C>T, c.-79A>G, c.92+5G>C, c.75T>A, c.316-2A>G, and/or c.316-2A>C).

Alterations of these sites may be made through the use of the genome editing systems disclosed herein. Genome editing systems, which are described in greater detail below, generally include an RNA-guided nuclease such as Cas9 or Cpf1 and a guide RNA that forms a complex with the RNA guided nuclease. The complex, in turn, may alter DNA in cells (or in vitro) in a site specific manner, directed by the targeting domain sequence of the gRNA. Alterations made by genome editing systems of this disclosure, which include (without limitation) single- and double-strand breaks, are discussed in greater detail below.

In certain embodiments of this disclosure, the alteration includes the insertion or replacement of a sequence in the HBB gene, which results in the transcription of a corrected HBB mRNA from the altered allele. For example, the alteration may include the targeted integration of a sequence comprising a region of an exon, or an entire exon, of the HBB gene in place of a mutation associated with SCD or β-thal. Alternatively or additionally, the alteration may include the insertion of a sequence comprising multiple exons of HBB into, e.g., an intronic sequence of the HBB gene. The inserted sequence may also comprise one or more of a splice donor sequence, a splice acceptor sequence, an intronic sequence, and/or a polyadenylation sequence. When inserted, the sequence results in the transcription of an mRNA encoding a functional HbB protein, which mRNA sequence may comprise only the inserted sequence, or it may comprise one or more unaltered HBB exons from the allele.

Genome editing systems used in these aspects and embodiments can be implemented in a variety of ways, as is discussed below in detail. As an example, a genome editing system of this disclosure can be implemented as a ribonucleoprotein complex or a plurality of complexes in which multiple gRNAs are used. This ribonucleoprotein complex can be introduced into a target cell using art-known methods, including electroporation, as described in commonly-assigned International Patent Publication No. WO 2016/182959 by Jennifer Gori (“Gori”), published Nov. 17, 2016, which is incorporated by reference in its entirety herein.

The ribonucleoprotein complexes within these compositions are introduced into target cells by art-known methods, including without limitation electroporation (e.g., using the Nucleofection™ technology commercialized by Lonza, Basel, Switzerland or similar technologies commercialized by, for example, Maxcyte Inc. Gaithersburg, Md.) and lipofection (e.g., using Lipofectamine™ reagent commercialized by Thermo Fisher Scientific, Waltham Mass.). Alternatively, or additionally, ribonucleoprotein complexes are formed within the target cells themselves following introduction of nucleic acids encoding the RNA-guided nuclease and/or gRNA. These and other delivery modalities are described in general terms below and in Gori.

Cells that have been altered ex vivo according to this disclosure can be manipulated (e.g., expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some cases, an autologous transplant includes the steps of obtaining, from the subject, a plurality of cells, either circulating in peripheral blood, or within the marrow or other tissue (e.g., spleen, skin, etc.), and manipulating those cells to enrich for cells in the erythroid lineage (e.g., by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system. The genome editing system can be implemented or delivered to the cells in any suitable format, including as a ribonucleoprotein complex, as separated protein and nucleic acid components, and/or as nucleic acids encoding the components of the genome editing system.

However it is implemented, a genome editing system may include, or may be co-delivered with, one or more factors that improve the viability of the cells during and after editing, including without limitation an aryl hydrocarbon receptor antagonist such as StemRegenin-1 (SRI), UMI71, LGC0006, alpha-napthoflavone, and CH-223191, and/or an innate immune response antagonist such as cyclosporin A, dexamethasone, reservatrol, a MyD88 inhibitory peptide, an RNAi agent targeting Myd88, a B18R recombinant protein, a glucocorticoid. OxPAPC, a TLR antagonist, rapamycin, BX795, and a RLR shRNA. These and other factors that improve the viability of the cells during and after editing are described in Gori, under the heading “I. Optimization of Stem Cells” from page 36 through page 61, which is incorporated by reference herein.

The cells, following delivery of the genome editing system, are optionally manipulated e.g., to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

Functionally, alteration of HBB using the compositions, methods and genome editing systems of this disclosure results in significant induction, among hemoglobin-expressing cells, of corrected 8-globin subunit protein (referred to interchangeably as HbB expression), e.g., at least 5%, 10%, 15%, 20%, 25%, 30%, 35%, 40%, 45%, 50% or greater induction of β subunit expression relative to unmodified controls. This induction of protein expression is generally the result of correction of the HBB gene by integration of a donor template (expressed, e.g., in terms of the percentage of total genomes comprising indel mutations within the plurality of cells) in some or all of the plurality of cells that are treated, e.g., at least 5%, 10%, 15%, 20%, 25%, 30%, 35%, 40%, 45%, 50% of the plurality of cells comprise at least one HBB allele comprising a corrected HBB sequence.

The functional effects of alterations caused or facilitated by the genome editing systems and methods of the present disclosure can be assessed in any number of suitable ways. For example, the effects of alterations on expression of β-globin can be assessed at the protein or mRNA level. Expression of HBB mRNA can be assessed by digital droplet PCR (ddPCR), which is performed on cDNA samples obtained by reverse transcription of mRNA harvested from treated or untreated samples. Primers for HBB, and other globin genes (e.g. HBA, HBG) may be used individually or multiplexed using methods known in the art. For example, ddPCR analysis of samples may be conducted using the QX200™ ddPCR system commercialized by Bio Rad (Hercules, Calif.), and associated protocols published by BioRad. Fetal hemoglobin protein may be assessed by high pressure liquid chromatography (HPLC), for example, according to the methods discussed on pp. 143-44 of Chang 2017, incorporated by reference herein, or fast protein liquid chromatography (FPLC) using ion-exchange and/or reverse phase columns to resolve HbF, HbB and HbA and/or γA and γG globin chains as is known in the art.

Donor template design is described in general terms below under the heading “HBB Donor Templates.”

While several of the exemplary embodiments above have focused on targeted integration at the HBB locus, it should be noted that other modifications of HBB and targeted integration of donor templates at other loci are within the scope of the present disclosure. These alterations may be catalyzed by an RNA-guided activity and/or by the recruitment of an endogenous factor to a target region.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

In certain embodiments, the genome editing systems in this disclosure may include a helicase for unwinding DNA. In certain embodiments, the helicase may be an RNA-guided helicase. In certain embodiments, the RNA-guided helicase may be an RNA-guided nuclease as described herein, such as a Cas9 or Cpf1 molecule. In certain embodiments, the RNA-guided nuclease is not configured to recruit an exogenous trans-acting factor to a target region. In certain embodiments, the RNA-guided nuclease may be configured to lack nuclease activity. In certain embodiments, the RNA-guided helicase may be complexed with a dead guide RNA as disclosed herein. For example, the dead guide RNA may comprise a targeting domain sequence less than 15 nucleotides in length. In certain embodiments, the dead guide RNA is not configured to recruit an exogenous trans-acting factor to a target region.

Naturally occurring CRISPR systems are organized evolutionarily into two classes and five types (Makarova 2011, incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as, without limitation, a lipid or polymer micro- or nano-particle, micelle, or liposome. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (see section below under the heading “Implementation of genome editing systems: delivery, formulations, and routes of administration”); and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino et al. (“Cotta-Ramusino”), which is incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, International Patent Publication No. WO 2015/070083 by Palestrant et al. (incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

As disclosed herein, in certain embodiments, genome editing systems may comprise multiple gRNAs that may be used to alter the HBB gene.

Genome editing systems can, in some instances, form double strand breaks that are repaired by cellular DNA double-strand break mechanisms such as NHEJ or HDR. These mechanisms are described throughout the literature (see, e.g., Davis 2014 (describing Alt-HDR), Frit 2014 (describing Alt-NHEJ), and Iyama 2013 (describing canonical HDR and NHEJ pathways generally), all of which are incorporated by reference herein).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added, the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor 2016, which is incorporated by reference herein. Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

### Guide RNA (RNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing), gRNAs and their component parts are described throughout the literature, for instance in Briner 2014, which is incorporated by reference), and in Cotta-Ramusino. Examples of modular and unimolecular gRNAs that may be used according to the embodiments herein include, without limitation, the sequences set forth in SEQ ID NOs:29-31 and 38-51. Examples of gRNA proximal and tail domains that may be used according to the embodiments herein include, without limitation, the sequences set forth in SEQ ID NOs:32-37.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end) (Mali 2013; Jiang 2013; Jinek 2012: all incorporated by reference herein).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner 2014) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes (Nishimasu et al., Cell 156, 935-949, Feb. 27, 2014 (“Nishimasu 2014”) and Nishimasu et al., Cell 162, 1113-1126, Aug. 27, 2015 (“Nishimasu 2015”), both incorporated by reference herein. It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain.” (Cotta-Ramusino) “stem loop 1” (Nishimasu 2014 and 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner 2014.

While the foregoing description has focused on gRNAs for use with Cas9, it should be appreciated that other RNA-guided nucleases exist which utilize gRNAs that differ in some ways from those described to this point. For instance, Cpf1 (“CRISPR from Prevotella and Franciscella 1”) is a recently discovered RNA-guided nuclease that does not require a tracrRNA to function (Zetsche 2015b, incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously (see, e.g., Mali 2013; Hsu 2013; Fu 2014: Heigwer 2014; Bae 2014; Xiao 2014; all incorporated by reference herein). As a non-limiting example, gRNA design may involve the use of a software tool to optimize the choice of potential target sequences corresponding to a user's target sequence. e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

Guide RNAs targeting the HBB gene, and methods of identifying the same, are described in WO/2015/148863 by Friedland, et al., (“Friedland”) under the heading “Strategies to identify gRNAs for S. pyogenes, S. Aureus, and N. meningitidis to correct a mutation in the HBB gene.” Individual guide RNA targeting domain sequences are provided in Tables 24A-D, 25A-B and 26 of Friedland. Friedland is incorporated by reference herein for all purposes.

qRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

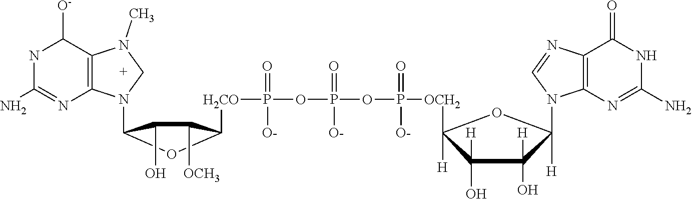

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

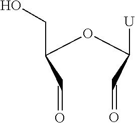

wherein “U” can be an unmodified or modified uridine.

The 3′ terminal U ribose can be modified with a 2′3′ cyclic phosphate as shown below:

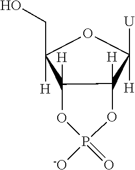

wherein “U” can be an unmodified or modified uridine.

Guide RNAs can contain 3′ nucleotides which can be stabilized against degradation. e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene): addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, 0- and N-alkylated nucleotides. e.g., N6-methyl adenosine, can be incorporated into the gRNA. In certain embodiments, one or more or all of the nucleotides in a gRNA are deoxynucleotides.

Dead gRNA Molecules

Dead guide RNA (dgRNA) molecules according to the present disclosure include, but are not limited to, dead guide RNA molecules that are configured such that they do not provide an RNA guided-nuclease cleavage event. For example, dead guide RNA molecules may comprise a targeting domain comprising 15 nucleotides or fewer in length. Dead guide RNAs may be generated by removing the 5′ end of a gRNA sequence, which results in a truncated targeting domain sequence. For example, if a gRNA sequence, configured to provide a cleavage event, has a targeting domain sequence that is 20 nucleotides in length, a dead guide RNA may be created by removing 5 nucleotides from the 5′ end of the gRNA sequence. In certain embodiments, the dead guide RNA is not configured to recruit an exogenous trans-acting factor to a target region. In certain embodiments, the dgRNA is configured such that it does not provide a DNA cleavage event when complexed with an RNA-guided nuclease. Skilled artisans will appreciate that dead guide RNA molecules may be designed to comprise targeting domains complementary to regions proximal to or within a target region in a target nucleic acid. In certain embodiments, dead guide RNAs comprise targeting domain sequences that are complementary to the transcription strand or non-transcription strand of double stranded DNA. The dgRNAs herein may include modifications at the 5′ and 3′ end of the dgRNA as described for guide RNAs in the section “gRNA modifications” herein. For example, in certain embodiments, dead guide RNAs may include an anti-reverse cap analog (ARCA) at the 5′ end of the RNA. In certain embodiments, dgRNAs may include a polyA tail at the 3′ end.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif.” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease). Examples of PAMs that may be used according to the embodiments herein include, without limitation, the sequences set forth in SEQ ID NOs: 199-205.

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above; see also Ran 2013, incorporated by reference herein), or that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The P1 domain, as its name suggests, contributes to PAM specificity. Examples of polypeptide sequences encoding Cas9 RuvC-like and Cas9 HNH-like domains that may be used according to the embodiments herein are set forth in SEQ ID NOs: 15-23 and 52-123 (RuvC-like domains) and SEQ ID NOs:24-28 and 124-198 (HNH-like domains).

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains). Examples of polypeptide sequences encoding Cas9 molecules that may be used according to the embodiments herein are set forth in SEQ ID NOs: 1-2, 4-6, 12, and 14.

The crystal structure of Acidaminococcus sp. Cpf1 in complex with crRNA and a double-stranded (ds) DNA target including a TTTN PAM sequence has been solved (Yamano 2016, incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique P1 domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity. PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran 2013 and Yamano 2016, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand, while inactivation of a Cas9 HNH domain results in a nickase that cleaves the bottom or non-complementary strand.

Modifications of PAM specificity relative to naturally occurring Cas9 reference molecules has been described for both S. pyogenes (Kleinstiver 2015a) and S. aureus (Kleinstiver 2015b). Modifications that improve the targeting fidelity of Cas9 have also been described (Kleinstiver 2016). Each of these references is incorporated by reference herein.

RNA-guided nucleases have been split into two or more parts (see, e.g., Zetsche 2015a; Fine 2015; both incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, which is incorporated by reference herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### RNA-Guided Helicases

RNA-guided helicases according to the present disclosure include, but are not limited to, naturally-occurring RNA-guided helicases that are capable of unwinding nucleic acid. As discussed supra, catalytically active RNA-guided nucleases cleave or modify a target region of DNA. It has also been shown that certain RNA-guided nucleases, such as Cas9, also have helicase activity that enables them to unwind nucleic acid. In certain embodiments, the RNA-guided helicases according to the present disclosure may be any of the RNA-nucleases described herein and supra in the section entitled “RNA-guided nucleases.” In certain embodiments, the RNA-guided nuclease is not configured to recruit an exogenous trans-acting factor to a target region. In certain embodiments, an RNA-guided helicase may be an RNA-guided nuclease configured to lack nuclease activity. For example, in certain embodiments, an RNA-guided helicase may be a catalytically inactive RNA-guided nuclease that lacks nuclease activity, but still retains its helicase activity. In certain embodiments, an RNA-guided nuclease may be mutated to abolish its nuclease activity (e.g., dead Cas9), creating a catalytically inactive RNA-guided nuclease that is unable to cleave nucleic acid, but which can still unwind DNA. In certain embodiments, an RNA-guided helicase may be complexed with any of the dead guide RNAs as described herein. For example, a catalytically active RNA-guided helicase (e.g., Cas9 or Cpf1) may form an RNP complex with a dead guide RNA, resulting in a catalytically inactive dead RNP (dRNP). In certain embodiments, a catalytically inactive RNA-guided helicase (e.g., dead Cas9) and a dead guide RNA may form a dRNP. These dRNPs, although incapable of providing a cleavage event, still retain their helicase activity that is important for unwinding nucleic acid.

### Nucleic Acids Encoding RNA-Guided Nucleases

Nucleic acids encoding RNA-guided nucleases, e.g., Cas9, Cpf1 or functional fragments thereof, are provided herein. Examples of nucleic acid sequences encoding Cas9 molecules that may be used according to the embodiments herein are set forth in SEQ ID NOs:3, 7-11, and 13. Exemplary nucleic acids encoding RNA-guided nucleases have been described previously (see, e.g., Cong 2013; Wang 2013: Mali 2013: Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped: polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA. e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art (see, e.g., Cotta-Ramusino). The stability of RNP complexes may be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g., different stoichiometric ratios of gRNA: RNA-guided nuclease protein, different buffer solutions, etc.) to identify optimal conditions for RNP formation; and (b) testing modifications (e.g., chemical modifications, alterations of sequence, etc.) of an RNA-guided nuclease and/or a gRNA to identify those modifications that improve RNP formation or stability. One readout of a DSF assay is a shift in melting temperature of the RNP complex: a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g., 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g., 2 μM) of Cas9 in water+10×SYPRO Orange® (Life Technologies cat # S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g., 2 μM) Cas9 in optimal buffer from assay 1 above and incubating (e.g., at RT for 10′) in a 384 well plate. An equal volume of optimal buffer+10×SYPRO Orange® (Life Technologies cat # S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e., to alter) targeted regions of DNA within or obtained from a cell. Various strategies are described herein to generate particular edits, and these strategies are generally described in terms of the desired repair outcome, the number and positioning of individual edits (e.g., SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region: or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g., a homologous sequence within the cellular genome), to promote gene conversion. Exogenous templates can have asymmetric overhangs (i.e., the portion of the template that is complementary to the site of the DSB may be offset in a 3′ or 5′ direction, rather than being centered within the donor template), for instance as described by Richardson 2016 (incorporated by reference herein). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g., a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g., ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

Genome editing systems according to this disclosure may also be employed for multiplex gene editing to generate two or more DSBs, either in the same locus or in different loci. Any of the RNA-guided nucleases and gRNAs disclosed herein may be used in genome editing systems for multiplex gene editing. Strategies for editing that involve the formation of multiple DSBs, or SSBs, are described in, for instance, Cotta-Ramusino.

As disclosed herein, multiple gRNAs may be used in genome editing systems to introduce alterations (e.g., deletions, insertions) into the HBB gene.

### HBB Donor Templates

Donor templates according to this disclosure may be implemented in any suitable way, including without limitation single stranded or double stranded DNA, linear or circular, naked or comprised within a vector, and/or associated, covalently or non-covalently (e.g. by direct hybridization or splint hybridization) with a guide RNA. In some embodiments, the donor template is a ssODN. Where a linear ssODN is used, it can be configured to (i) anneal to a nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, or no more than 150-200 nucleotides (e.g., 150, 160, 170, 180, 190, or 200 nucleotides). In other embodiments, the donor template is a dsODN. In one embodiment, the donor template comprises a first strand. In another embodiment, a donor template comprises a first strand and a second strand. In some embodiments, a donor template is an exogenous oligonucleotide, e.g., an oligonucleotide that is not naturally present in a cell.

It should be noted that a donor template can also be comprised within a nucleic acid vector, such as a viral genome or circular double-stranded DNA, e.g., a plasmid. In some embodiments, the donor template can be a doggy-bone shaped DNA (see, e.g., U.S. Pat. No. 9,499,847). Nucleic acid vectors comprising donor templates can include other coding or non-coding elements. For example, a donor template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

A. Homology Arms

Whether single-stranded or double-stranded, donor templates generally include one or more regions that are homologous to regions of DNA. e.g., a target nucleic acid, within or near (e.g., flanking or adjoining) a target sequence to be cleaved, e.g. the cleavage site. These homologous regions are referred to here as “homology arms,” and are illustrated schematically below:

[5′ homology arm]-[replacement sequence]-[3′ homology arm].

The homology arms of the donor templates described herein may be of any suitable length, provided such length is sufficient to allow efficient resolution of a cleavage site on a targeted nucleic acid by a DNA repair process requiring a donor template. In some embodiments, where amplification by, e.g. PCR, of the homology arm is desired, the homology arm is of a length such that the amplification may be performed. In some embodiments, where sequencing of the homology arm is desired, the homology arm is of a length such that the sequencing may be performed. In some embodiments, where quantitative assessment of amplicons is desired, the homology arms are of such a length such that a similar number of amplifications of each amplicon is achieved, e.g., by having similar G/C content, amplification temperatures, etc. In some embodiments, the homology arm is double-stranded. In some embodiments, the double stranded homology arm is single stranded.

In some embodiments, the 5′ homology arm is between 150 to 250 nucleotides in length. In some embodiments, the 5′ homology arm is 700 nucleotides or less in length. In some embodiments, the 5′ homology arm is 650 nucleotides or less in length. In some embodiments, the 5′ homology arm is 600 nucleotides or less in length. In some embodiments, the 5′ homology arm is 550 nucleotides or less in length. In some embodiments, the 5′ homology arm is 500 nucleotides or less in length. In some embodiments, the 5′ homology arm is 400 nucleotides or less in length. In some embodiments, the 5′ homology arm is 300 nucleotides or less in length. In some embodiments, the 5′ homology arm is 250 nucleotides or less in length. In some embodiments, the 5′ homology arm is 200 nucleotides or less in length. In some embodiments, the 5′ homology arm is 150 nucleotides or less in length. In some embodiments, the 5′ homology arm is less than 100 nucleotides in length. In some embodiments, the 5′ homology arm is 50 nucleotides in length or less. In some embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the 5′ homology arm is 40 nucleotides in length. In some embodiments, the 3′ homology arm is 250 nucleotides in length or less.

In some embodiments, the 3′ homology arm is between 150 to 250 nucleotides in length. In some embodiments, the 3′ homology arm is 700 nucleotides or less in length. In some embodiments, the 3′ homology arm is 650 nucleotides or less in length. In some embodiments, the 3′ homology arm is 600 nucleotides or less in length. In some embodiments, the 3′ homology arm is 550 nucleotides or less in length. In some embodiments, the 3′ homology arm is 500 nucleotides or less in length. In some embodiments, the 3′ homology arm is 400 nucleotides or less in length. In some embodiments, the 3′ homology arm is 300 nucleotides or less in length. In some embodiments, the 3′ homology arm is 200 nucleotides in length or less. In some embodiments, the 3′ homology arm is 150 nucleotides in length or less. In some embodiments, the 3′ homology arm is 100 nucleotides in length or less. In some embodiments, the 3′ homology arm is 50 nucleotides in length or less. In some embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the 3′ homology arm is 40 nucleotides in length.

In some embodiments, the 5′ homology arm is between 150 basepairs to 250 basepairs in length. In some embodiments, the 5′ homology arm is 700 basepairs or less in length. In some embodiments, the 5′ homology arm is 650 basepairs or less in length. In some embodiments, the 5′ homology arm is 600 basepairs or less in length. In some embodiments, the 5′ homology arm is 550 basepairs or less in length. In some embodiments, the 5′ homology arm is 500 basepairs or less in length. In some embodiments, the 5′ homology arm is 400 basepairs or less in length. In some embodiments, the 5′ homology arm is 300 basepairs or less in length. In some embodiments, the 5′ homology arm is 250 basepairs or less in length. In some embodiments, the 5′ homology arm is 200 basepairs or less in length. In some embodiments, the 5′ homology arm is 150 basepairs or less in length. In some embodiments, the 5′ homology arm is less than 100 basepairs in length. In some embodiments, the 5′ homology arm is 50 basepairs in length or less. In some embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 basepairs in length. In some embodiments, the 5′ homology arm is 40 basepairs in length. In some embodiments, the 3′ homology arm is 250 basepairs in length or less. In some embodiments, the 3′ homology arm is 200 basepairs in length or less. In some embodiments, the 3′ homology arm is 150 basepairs in length or less. In some embodiments, the 3′ homology arm is 100 basepairs in length or less. In some embodiments, the 3′ homology arm is 50 basepairs in length or less. In some embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 basepairs in length. In some embodiments, the 3′ homology arm is 40 basepairs in length.

The 5′ and 3′ homology arms can be of the same length or can differ in length. In some embodiments, the 5′ and 3′ homology arms are amplified to allow for the quantitative assessment of gene editing events, such as targeted integration, at a target nucleic acid. In some embodiments, the quantitative assessment of the gene editing events may rely on the amplification of both the 5′ junction and 3′ junction at the site of targeted integration by amplifying the whole or a part of the homology arm using a single pair of PCR primers in a single amplification reaction. Accordingly, although the length of the 5′ and 3′ homology arms may differ, the length of each homology arm should be capable of amplification (e.g., using PCR), as desired. Moreover, when amplification of both the 5′ and the difference in lengths of the 5′ and 3′ homology arms in a single PCR reaction is desired, the length difference between the 5′ and 3′ homology arms should allow for PCR amplification using a single pair of PCR primers.

In some embodiments, the length of the 5′ and 3′ homology arms does not differ by more than 75 nucleotides. Thus, in some embodiments, when the 5′ and 3′ homology arms differ in length, the length difference between the homology arms is less than 70, 60, 50, 40, 30, 20, 10, 9, 8, 7, 6, 5, 4, 3, 2, 1 nucleotides or base pairs. In some embodiments, the 5′ and 3′ homology arms differ in length by at least 1, 2, 3, 4, 5, 6, 7, 8, 9, 10, I 1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In some embodiments, the length difference between the 5′ and 3′ homology arms is less than 70, 60, 50, 40, 30, 20, 10, 9, 8, 7, 6, 5, 4, 3, 2, 1 base pairs. In some embodiments, the 5′ and 3′ homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs.

Donor templates of the disclosure are designed to facilitate homologous recombination with a target nucleic acid having a cleavage site, wherein the target nucleic acid comprises, from 5′ to 3′,

P1-H1--X--H2--P2,

wherein P1 is a first priming site; H1 is a first homology arm; X is the cleavage site; H2 is a second homology arm; and P2 is a second priming site; and wherein the donor template comprises, from 5′ to 3′,

A1--P2′--N--A2, or A1--N--P1′--A2,

wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2. In one embodiment, the target nucleic acid is double stranded. In one embodiment, the target nucleic acid comprises a first strand and a second strand. In another embodiment, the target nucleic acid is single stranded. In one embodiment, the target nucleic acid comprises a first strand.

In some embodiments, the donor template comprises, from 5′ to 3′,

A1--P2′--N--A2.

In some embodiments, the donor template comprises, from 5′ to 3′,

A1--P2′--N--P1′--A2.

In some embodiments, the target nucleic acid comprises, from 5′ to 3′.

P1-H1--X--H2--P2,

wherein P1 is a first priming site; H1 is a first homology arm; X is the cleavage site; H2 is a second homology arm; and P2 is a second priming site; and the first strand of the donor template comprises, from 5′ to 3′.

A1--P2′--N--A2, or A1--N--P1′--A2,

wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2.

In some embodiments, a first strand of the donor template comprises, from 5′ to 3′,

A1--P2′--N--P1′--A2.

In some embodiments, a first strand of the donor template comprises, from 5′ to 3′,

A1--N--P1′-A2.

In some embodiments, A1 is 700 basepairs or less in length. In some embodiments, A1 is 650 basepairs or less in length. In some embodiments, A1 is 600 basepairs or less in length. In some embodiments, A1 is 550 basepairs or less in length. In some embodiments, A1 is 500 basepairs or less in length. In some embodiments, A1 is 400 basepairs or less in length. In some embodiments, A1 is 300 basepairs or less in length. In some embodiments, A1 is less than 250 base pairs in length. In some embodiments, A1 is less than 200 base pairs in length. In some embodiments, A1 is less than 150 base pairs in length. In some embodiments, A1 is less than 100 base pairs in length. In some embodiments, A1 is less than 50 base pairs in length. In some embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In some embodiments, A1 is 40 base pairs in length. In some embodiments, A1 is 30 base pairs in length. In some embodiments, A1 is 20 base pairs in length.

In some embodiments. A2 is 700 basepairs or less in length. In some embodiments, A2 is 650 basepairs or less in length. In some embodiments, A2 is 600 basepairs or less in length. In some embodiments, A2 is 550 basepairs or less in length. In some embodiments, A2 is 500 basepairs or less in length. In some embodiments, A2 is 400 basepairs or less in length. In some embodiments, A2 is 300 basepairs or less in length. In some embodiments, A2 is less than 250 base pairs in length. In some embodiments, A2 is less than 200 base pairs in length. In some embodiments, A2 is less than 150 base pairs in length. In some embodiments, A2 is less than 100 base pairs in length. In some embodiments. A2 is less than 50 base pairs in length. In some embodiments. A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In some embodiments, A2 is 40 base pairs in length. In some embodiments, A2 is 30 base pairs in length. In some embodiments, A2 is 20 base pairs in length.

In some embodiments, A1 is 700 nucleotides or less in length. In some embodiments, A1 is 650 nucleotides or less in length. In some embodiments, A1 is 600 nucleotides or less in length. In some embodiments, A1 is 550 nucleotides or less in length. In some embodiments, A1 is 500 nucleotides or less in length. In some embodiments, A1 is 400 nucleotides or less in length. In some embodiments, A1 is 300 nucleotides or less in length. In some embodiments, A1 is less than 250 nucleotides in length. In some embodiments, A1 is less than 200 nucleotides in length. In some embodiments, A1 is less than 150 nucleotides in length. In some embodiments. A1 is less than 100 nucleotides in length. In some embodiments, A1 is less than 50 nucleotides in length. In some embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, A1 is at least 40 nucleotides in length. In some embodiments, A1 is at least 30 nucleotides in length. In some embodiments, A1 is at least 20 nucleotides in length.

In some embodiments, A2 is 700 nucleotides or less in length. In some embodiments, A2 is 650 basepairs or less in length. In some embodiments, A2 is 600 nucleotides or less in length. In some embodiments, A2 is 550 nucleotides or less in length. In some embodiments, A2 is 500 nucleotides or less in length. In some embodiments, A2 is 400 nucleotides or less in length. In some embodiments. A2 is 300 nucleotides or less in length. In some embodiments, A2 is less than 250 nucleotides in length. In some embodiments, A2 is less than 200 nucleotides in length. In some embodiments, A2 is less than 150 nucleotides in length. In some embodiments, A2 is less than 100 nucleotides in length. In some embodiments, A2 is less than 50 nucleotides in length. In some embodiments. A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, A2 is at least 40 nucleotides in length. In some embodiments, A2 is at least 30 nucleotides in length. In some embodiments, A2 is at least 20 nucleotides in length.

In some embodiments, the nucleic acid sequence of A1 is substantially identical to the nucleic acid sequence of H1. In some embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H1. In some embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H1.

In some embodiments, the nucleic acid sequence of A2 is substantially identical to the nucleic acid sequence of H2. In some embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H2. In some embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H2.

Whatever format is used, a donor template can be designed to avoid undesirable sequences. In certain embodiments, one or both homology arms can be shortened to avoid overlap with certain sequence repeat elements, e.g., Alu repeats, LINE elements, etc.

B. Priming Sites

The donor templates described herein comprise at least one priming site having a sequence that is substantially similar to, or identical to, the sequence of a priming site within the target nucleic acid, but is in a different spatial order or orientation relative to a homology sequence/homology arm in the donor template. When the donor template is homologously recombined with the target nucleic acid, the priming site(s) are advantageously incorporated into the target nucleic acid, thereby allowing for the amplification of a portion of the altered nucleic acid sequence that results from the recombination event. In some embodiments, the donor template comprises at least one priming site. In some embodiments, the donor template comprises a first and a second priming site. In some embodiments, the donor template comprises three or more priming sites.

In some embodiments, the donor template comprises a priming site P1′, that is substantially similar or identical to a priming site. P1, within the target nucleic acid, wherein upon integration of the donor template at the target nucleic acid, P1′ is incorporated downstream from P1. In some embodiments, the donor template comprises a first priming site, P1′, and a second priming site, P2′: wherein P1′ is substantially similar or identical to a first priming site, P1, within the target nucleic acid: wherein P2′ is substantially similar or identical to second priming site, P2, within the target nucleic acid; and wherein P1 and P2 are not substantially similar or identical. In some embodiments, the donor template comprises a first priming site, P1′, and a second priming site, P2′; wherein P1′ is substantially similar or identical to a first priming site, P1, within the target nucleic acid; wherein P2′ is substantially similar or identical to second priming site, P2, within the target nucleic acid; wherein P2 is located downstream from P1 on the target nucleic acid; wherein P1 and P2 are not substantially similar or identical; and wherein upon integration of the donor template at the target nucleic acid, P1′, is incorporated downstream from P1. P2′ is incorporated upstream from P2, and P2′ is incorporated upstream from P1.

In some embodiments, the target nucleic acid comprises a first priming site (P1) and a second priming site (P2). The first priming site in the target nucleic acid may be within the first homology arm. Alternatively, the first priming site in the target nucleic acid may be 5′ and adjacent to the first homology arm. The second priming site in the target nucleic acid may be within the second homology arm. Alternatively, the second priming site in the target nucleic acid may be 3′ and adjacent to the second homology arm.

The donor template may comprise a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to P1, and wherein P2′ is substantially identical to P2. In this scenario, a primer pair comprising an oligonucleotide targeting P1′ and P1 and an oligonucleotide comprising P2′ and P2 may be used to amplify the targeted locus, thereby generation three amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Y, results from the amplification of the nucleic acid sequence between P and P2′ following a targeted integration event at the target nucleic acid, thereby amplifying the 5′ junction. The third amplicon. Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover, P2′ may be identical to P2.

In some embodiments, the donor template comprises a cargo and a priming site (P1′), wherein P1′ is located 3′ from the cargo nucleic acid sequence (i.e., A1--N--P1′-A2) and P1′ is substantially identical to P1. In this scenario, a primer pair comprising an oligonucleotide targeting P1′ and P1 and an oligonucleotide targeting P2 may be used to amplify the targeted locus, thereby generation two amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover. P2′ may be identical to P2.

In some embodiments, the target nucleic acid comprises a first priming site (P1) and a second priming site (P2), and the donor template comprises a priming site P2′, wherein P2′ is located 5′ from the cargo nucleic acid sequence (i.e., A1--P2′--N--A2), and P2′ is substantially identical to P2. In this scenario, a primer pair comprising an oligonucleotide targeting P2′ and P2 and an oligonucleotide targeting P1 may be used to amplify the targeted locus, thereby generation two amplicons of similar size which may be sequenced to determine whether targeted integration has occurred. The first amplicon. Amplicon X, results from the amplification of the nucleic acid sequence between P1 and P2 as a result of non-targeted integration at the target nucleic acid. The second amplicon, Amplicon Y, results from the amplification of the nucleic acid sequence between P and P2′ following a targeted integration event at the target nucleic acid, thereby amplifying the 5′ junction. In other embodiments, P1′ may be identical to P1. Moreover, P2′ may be identical to P2.

A priming site of the donor template may be of any length that allows for the quantitative assessment of gene editing events at a target nucleic acid by amplication and/or sequencing of a portion of the target nucleic acid. For example, in some embodiments, the target nucleic acid comprises a first priming site (P1) and the donor template comprises a priming site (P1′). In these embodiments, the length of the P1′ priming site and the P1 primer site is such that a single primer can specifically anneal to both priming sites (for example, in some embodiments, the length of the P1′ priming site and the P1 priming site is such that both have the same or very similar GC content).

In some embodiments, the priming site of the donor template is 60 nucleotides in length. In some embodiments, the priming site of the donor template is less than 60 nucleotides in length. In some embodiments, the priming site of the donor template is less than 50 nucleotides in length. In some embodiments, the priming site of the donor template is less than 40 nucleotides in length. In some embodiments, the priming site of the donor template is less than 30 nucleotides in length. In some embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides in length. In some embodiments, the priming site of the donor template is 60 base pairs in length. In some embodiments, the priming site of the donor template is less than 60 base pairs in length. In some embodiments, the priming site of the donor template is less than 50 base pairs in length. In some embodiments, the priming site of the donor template is less than 40) base pairs in length. In some embodiments, the priming site of the donor template is less than 30 base pairs in length. In some embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 base pairs in length.

In some embodiments, upon resolution of the cleavage event at the cleavage site in the target nucleic acid and homologous recombination of the donor template with the target nucleic acid, the distance between the first priming site of the target nucleic acid (P1) and now integrated P2′ priming site is 600 base pairs or less. In some embodiments, upon resolution of the cleavage event and homologous recombination of the donor template with the target nucleic acid, the distance between the first priming site of the target nucleic acid (P1) and now integrated P2′ priming site is 550, 500, 450, 400, 350, 300, 250, 200, 150 base pairs or less. In some embodiments, upon resolution of the cleavage event at the target nucleic acid and homologous recombination of the donor template with the target nucleic acid, the distance between the first priming site of the target nucleic acid (P1) and now integrated P2′ priming site is 600 nucleotides or less. In some embodiments, upon resolution of the cleavage event at the target nucleic acid and homologous recombination of the donor template with the target nucleic acid, the distance between the first priming site of the target nucleic acid (P1) and now integrated P2′ priming site is 550, 500, 450, 400, 350, 300, 250, 200, 150 nucleotides or less.

In some embodiments, the target nucleic acid comprises a second priming site (P2) and the donor template comprises a priming site (P2′) that is substantially identical to P2. In some embodiments, upon resolution of the cleavage event at the target nucleic acid and homologous recombination of the donor template with the target nucleic acid, the distance between the second priming site of the target nucleic acid (P2) and now integrated P1′ priming site is 600 base pairs or less. In some embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 base pairs or less. In some embodiments, upon resolution of the cleavage event at the target nucleic acid and homologous recombination of the donor template with the target nucleic acid, the distance between the second priming site of the target nucleic acid (P2) and now integrated P1′ priming site is 600 nucleotides or less. In some embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 nucleotides or less.

In some embodiments, the nucleic acid sequence of P2′ is comprised within the nucleic acid sequence of A1. In some embodiments, the nucleic acid sequence of P2′ is immediately adjacent to the nucleic acid sequence of A1. In some embodiments, the nucleic acid sequence of P2′ is immediately adjacent to the nucleic acid sequence of N. In some embodiments, the nucleic acid sequence of P2′ is comprised within the nucleic acid sequence of N.

In some embodiments, the nucleic acid sequence of P1′ is comprised within the nucleic acid sequence of A2. In some embodiments, the nucleic acid sequence of P1′ is immediately adjacent to the nucleic acid sequence of A2. In some embodiments, the nucleic acid sequence of P1′ is immediately adjacent to the nucleic acid sequence of N. In some embodiments, the nucleic acid sequence of P1′ is comprised within the nucleic acid sequence of N.

In some embodiments, the nucleic acid sequence of P2′ is comprised within the nucleic acid sequence of S1. In some embodiments, the nucleic acid sequence of P2′ is immediately adjacent to the nucleic acid sequence of S1. In some embodiments, the nucleic acid sequence of P1′ is comprised within the nucleic acid sequence of S2. In some embodiments, the nucleic acid sequence of P1′ is immediately adjacent to the nucleic acid sequence of S2.

C. Cargo

The donor template of the gene editing systems described herein comprises a cargo (N). The cargo may be of any length necessary in order to achieve the desired outcome. For example, a cargo sequence may be less than 2500 base pairs or less than 2500) nucleotides in length. Those of skill in the art will readily ascertain that when the donor template is delivered using a delivery vehicle (e.g., a viral delivery vehicle such as an adeno-associated virus (AAV) or herpes simplex virus (HSV) delivery vehicle) with size limitations, the size of the donor template, including cargo, should not exceed the size limitation of the delivery system.

In some embodiments, the cargo comprises a replacement sequence. In some embodiments, the cargo comprises an exon of a gene sequence. In some embodiments, the cargo comprises an intron of a gene sequence. In some embodiments, the cargo comprises a cDNA sequence. In some embodiments, the cargo comprises a transcriptional regulatory element. In some embodiments, the cargo comprises a reverse complement of a replacement sequence, an exon of a gene sequence, an intron of a gene sequence, a cDNA sequence or a transcriptional regulatory element. In some embodiments, the cargo comprises a portion of a replacement sequence, an exon of a gene sequence, an intron of a gene sequence, a cDNA sequence or a transcriptional regulatory element.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

D. Stuffers

In some embodiments, the donor template may optionally comprise one or more stuffer sequences. Generally, a stuffer sequence is a heterologous or random nucleic acid sequence that has been selected to (a) facilitate (or to not inhibit) the targeted integration of a donor template of the present disclosure into a target site and the subsequent amplification of an amplicon comprising the stuffer sequence according to certain methods of this disclosure, but (b) to avoid driving integration of the donor template into another site. The stuffer sequence may be positioned, for instance, between a homology arm A1 and a primer site P2′ to adjust the size of the amplicon that will be generated when the donor template sequence is interated into the target site. Such size adjustments may be employed, as one example, to balance the size of the amplicons produced by integrated and non-integrated target sites and, consequently to balance the efficiencies with which each amplicon is produced in a single PCR reaction; this in turn may facilitate the quantitative assessment of the rate of targeted integration based on the relative abundance of the two amplicons in a reaction mixture.

To facilitate targeted integration and amplification, the stuffer sequence may be selected to minimize the formation of secondary structures which may interfere with the resolution of the cleavage site by the DNA repair machinery (e.g., via homologous recombination) or which may interfere with amplification. In some embodiments, the donor template comprises, from 5′ to 3′,

A1--S1--P2′--N--A2, or

A1--N--P1′--S2--A2:

wherein S1 is a first stuffer sequence and S2 is a second stuffer sequence.

In some embodiments, the donor template comprises from 5′ to 3′,

### A1--S1--P2′-N--P1′-S2--A2,

wherein S1 is a first stuffer sequence and S2 is a second stuffer sequence.

In some embodiments, the stuffer sequence comprises about the same guanine-cytosine content (“GC content”) as the genome of the cell as a whole. In some embodiments, the stuffer sequences comprises about the same GC content as the targeted locus. For example, when the target cell is a human cell, the stuffer sequence comprises about 40% GC content. In some embodiments, a stuffer sequence may be designed by generating random nucleic acid sequence sequences comprising the desired GC content. For example, to generate a stuffer sequence comprising 40% GC content, nucleic acid sequences having the following distribution of nucleotides may be designed: A=30%, T=30%, G=20%, C=20%. Methods for determining the GC content of the genome or the GC content of the target locus are known to those of skill in the art. Thus, in some embodiments, the stuffer sequence comprises 20%, 25%, 30%, 35%, 40%, 45%, 50%, 55% 60%, 65%, 70%, or 75% GC content. Exemplary 2.0 kilobase stuffer sequences having 40±5% GC content are provided in Table 2.

TABLE 2
Exemplary 2.0 Kilobase Stuffer Sequences Having 40 ± 5% GC Content

| SEQ | | |
|---|---|---|
| ID | GC | |
| NO. | Content | Stuffer Sequence |
| 251 | 38.40% | TCAATAGCCCAGTCGGTTTTGTTAGATACATTTTATCGAATCTGTAAAGATATTTTATAAT |
| | | AAGATAATATCAGCGCCTAGCTGCGGAATTCCACTCAGAGAATACCTCTCCTGAATATCAG |
| | | CCTTAGTGGCGTTATACGATATTTCACACTCTCAAAATCCCGAGTCAGACTATACCCGCGC |
| | | ATGTTTAGTAAAGGTTGATTCTGAGATCTCGAGTCCAAAAAAGATACCCACTACTTTAAAG |
| | | ATTTGCATTCAGTTGTTCCATCGGCCTGGGTAGTAAAGGGGGTATGCTCGCTCCGAGTCGA |
| | | TGGAACTGTAAATGTTAGCCCTGATACGCGGAACATATCAGTAACAATCTTTACCTAATAT |
| | | GGAGTGGGATTAAGCTTCATAGAGGATATGAAACGCTCGTAGTATGGCTTCCTACATAAGT |
| | | AGAATTATTAGCAACTAAGATATTACCACTGCCCAATAALAGAGATTCCACTTAGATTCAT |
| | | AGGTAGTCCCAACAATCATGTCTGAATACTAAATTGATCAATTGGACTATGTCAAAATTAT |
| | | TTTGAAGAAGTAATCATCAACTTAGGCGCTTTTTAGTGTTAAGAGCGCGTTATTGCCAACC |
| | | GGGCTAAACCTGTGTAACTCTTCAATATTGTATATAATTATAGGCAGAATAAGCTATGAGT |
| | | GCATTATGAGATAAACATAGATTTTTGTCCACTCGAAATATTTaAATTTCTTGATCCTGGG |
| | | CTAGTTCAGCCATAAGTTTTCACTAATAGTTAGGACTACCAATTACACTACATTCAGTTGC |
| | | TGAAATTCACATCACTGCCGCAATATTTATGAAGCTATTATTGCATTAAGACTTAGGAGAT |
| | | AAATACGAAGTTGATATATTTTTCAGAATCAGCGAAAAGACCCCCTATTGACATTACGAAT |
| | | TCGAGTTTAACGAGCACATAAATCAAACACTACGAGGTTACCAAGATTGTATCTTACATTA |
| | | ATGCTATCCAGCCAGCCGTCATGTTTAACTGGATAGTCATAATTAATATCCAATGATCGTT |
| | | TCACGTAGCTGCATATCGAGGAAGTTGTATAATTGAAAACCCACACATTAGAATGCATGGT |
| | | GCATCGCTAGGGTTTATCTTATCTTGCTCGTGCCAAGAGTGTAGAAAGCCACATATTGATA |
| | | CGGAAGCTGCCTAGGAGGTTGGTATATGTTGATTGTGCTCACCATCTCCCTTCCTAATCTC |
| | | CTAGTGTTAAGTCCAATCAGTGGGCTGGCTCTGGTTAAAAGTAATATACACGCTAGATCTC |
| | | TCTACTATAATACAGGCTAAGCCTACGCGCTTTCAATGCACTGATTACCAACTTAGCTACG |
| | | GCCAGCCCCATTTAATGAATTATCTCAGATGAATTCAGACATTATTCTCTACAAGGACACT |
| | | TTAGAGTGTCCTGOGGAGGCATAATTATTATCTAAGATGGGGTAAGTCCGATGGAAGACAC |
| | | AGATACATCGGACTATTCCTATTAGCCGAGAGTCAACCGTTAGAACTCGGAAAAAGACATC |
| | | GAAGCCGGTAACCTACGCACTATAAATTTCCGCAGAGACATATGTAAAGTTTTATTAGAAC |
| | | TGGTATCTTGATTACGATTCTTAACTCTCATACGCCGGTCCGGAATTTGTGACTCGAGAAA |
| | | ATGTAATGACATGCTCCAATTGATTTCAAAATTAGATTTAAGGTCAGCGAACTATGTTTAT |
| | | TCAACCGTTTACAACGCTATTATGCGCGATGGATGGGGCCTTGTATCTAGAAACCGAATAA |
| | | TAACATACCTGTTAAATGGCAAACTTAGATTATTGCGATTAATTCTCACTTCAGAGGGTTA |
| | | TCGTGCCGAATTCCTGACTTTGGAATAATAAAGTTGATATTGAGGTGCAATATCAACTACA |
| | | CTGGTTTAACCTTTAAACACATGGAGTCAAGTTTTCGCTATGCCAGCCGGTTATGCAGCTA |
| | | GGATTAATATTAGAGCTCTTTTCTAATTCGTCCTAATAATCTCTTCAC |
| 252 | 38.90% | AAAACGTACTACGTCCACTAATATAGTGCTCAGGGCCTTTAAAGTTATGAACAGGAATACG |
| | | GCGATGACGATAGAGATGTACAACTCAGTGCGAACCCCAGTGTATGTACAAAAAGTTACTA |
| | | ATTCACTTTACTGTTTTGAGGATGTACCTGCCAAAAAGATTCAGATTATGAAAGTCAGATC |
| | | TTTATATGACGGAACGCGCAAAGGATCCTATTAGGATGCGCCTCAAAAAGCCATCTAAAAA |
| | | GTTCATGTATTGAGCTTATTAGTAAAGGTATCAACAAAAATGATTCCACCTTATATAAATA |
| | | AGCTTGATCCCATTAATTGAATAATAAAGACCGAGTAATCACTTTTATGCATGTAACAAAA |
| | | ATCCCGTTTGCGGCTATGCTACAACGGTCATCCCATAGAATATTATCATCGTACAAGCCCA |
| | | AGACCCGATGCTCAACATTAGAGCCAAATAACGTGCACACTCCTAATATGAGATGACTGCC |
| | | GCTTTTAACACCAGATCTGTTAGTTAGGCCACGCACTTCCAAGTTTATCTAGAGTGCATGT |
| | | CTTTATATATGTTGGTCCCCTGTAATGACTTATAATATTTCCTTCGACTGTGTTGAACATC |
| | | TGTAACAATAAAGACTAAAGCTCTGGGTATATAAGGTTGCAGTGGTACCTTATTAGGTCCA |
| | | TTATCGCAGAATACTGCGGATGGACAATCTTGCCAATTTAATTGACTATCTATTAGTTTGC |
| | | ACAATATAACGATTCGTCTTGGACAAATTTGGCGAGTGAGCCCCTTACTCGCTCAAAATGT |
| | | TACAATTGCCGAGCTCGGAGTTGAATGATTAGTTACATATTATAGAACAGAATGCAGATGT |
| | | AGTTAGACAAGATGTGTTGATGAATGTGAAGTCTGACTGGAGTAAAGGAACAAGAGCACCC |
| | | ACCTACGTATATTGCGCATTTTAAATGTAGCCTCGACTCTAACACGTGCGACGTGAGTCAT |
| | | AATTGTGCATGTTATTAGATCTATGGAATGTTGTTTTTTTAATTATCAAACGTACGTCAAA |
| | | CCGCCAAACTCCGTGTGCCATAGAGTATACTCCTGAAGTTCGAAATTAGGCCATAAAGTCT |
| | | TTCTTGCTGGTTGTGAAATGAAGGGGTGTTTCATAATTTAACTTTGACTGCTTCTGTTGGG |
| | | ACGACGTACCCGTTCGTTTGTTTGTCCTACTATTTAGTATCTTAAAACAGTCCATTTACCG |
| | | TTAATGTTCTTAACCCTTAAAGATACAAACTTAGCTCTGTAATCAACTTCAAGACGTCTTT |
| | | GACAGAACGTCTAAGACCCAGATCTGTGTTAGCCAACTCGTATTCAATTTCGTACCGGTGG |
| | | ACTTCGGCCCCTCACACTGCCATTAGTTGATGCTGAACTTTGTATTTGCTGGGTAGGATAT |
| | | ATAACGATTTTGCAGATGTGTGTGCTAAGTATATTGTCTTAGTGACGGTCCAGCATATAAA |
| | | ACACCTACACAAGAAGGTTATTCTTAATGGTTGATTGAATATTATTAAATTGTTGCTTTTA |
| | | CTTTTTCCTCCTACAAATTGTCATGAGCTCAAATTTGTTGACCTAAGGTATTAATATTGTA |
| | | TCCTACACGGATTGTGAACGGTAGGGTCGTAACAATCGTACTTTACGGCTTAAAAATTGTA |
| | | AGCACCTTGCCAGGTAGATGAAAACTTTAAGGATAGAAGTATAGTAACTCACATGCTTGCG |
| | | GCAGCATCGTAGGGCAGAGGTGTGATCTTGGTGATTGAAATTAAGGGGTAGGATGATCGGC |
| | | CGCATATATCGGCTACTAGGATTAGATAGATGCAACGCTTTACTTTAATCAAGTGACGTCC |
| | | GTATAAGTAAGACATCTAATGGCTGTATTTTTGTATACAAGTATAAGGAACCGGGGAGTCT |
| | | TTATAGCGACGCGTAATTATATATTCCAAATCAGTTAAGTGGCGTCGGTTACGAAACTAAA |
| | | GAGAGTGTTCAAGACGCAATGAAGAATCGTGAGCGTAATTGTTCGCGC |
| 253 | 39.30% | AACCCTCGTGTCCGGTAAAACACGCTTCGAATACAAAAGATTATATAGGTACGGAAGGCTG |
| | | GGAATCTTTCTTCGATGGAACTGAGATTATATTCCACTGTAACCTTATTATGACTATAGAT |
| | | TTCCAACATACGGATAGATTAATACCGACTGTAGATTCCATACTTGAACTATGAAGCCGTA |
| | | CGAGTACCCATACTATAACTAAGACTATGACACGTGTGAATTCGTGTTTATCATAGTGCAA |
| | | ACTCTTGCTATTCCACATGGGAGTTTAGAACTCAGCTGTTCCTATACAATTAGCACTACAA |
| | | ACCCACTAATATGGATAGCATGATACCATCTGAGGAGGATTTGGTGTTACCATGTTGTAAT |
| | | CTAAGAAGTTTCACAAAATCAACGTTAGATAAACGGCAATATACGCGCACTAATAATGAAC |
| | | CCCAAGATATCAGTTGAAAAATTTTCGATCTCCTCTTTAAATTAACAAATATTGCAGAGTA |
| | | AGTACCGAAATTGTGACACAAGTGCCGTTTGCCCGTCTTTTTCACAGCCTATAAAGTTCAG |
| | | ATCTATATGGGCTCCCACTTAACCTTCAGATAGATAACAAGTTACTGGAAGTGATTCTATC |
| | | ATAATACAATCAACTATAACACATCCAATGATATATCTCGAGAAAGTCGTAGTCTAGAGCT |
| | | CCTTCTATTATCCGGTCTTACCTAAATAGTTATATTTAGTTGCCCATTTAAAATTGGATAG |
| | | GAGGAGGGGTGCTCATGATTTAAAAACCAACTGTGCATGCGGTTCTTTGATGTGGATCCAC |
| | | CTTGCAAAGCGCTAAAGATAAAAGTAGTCACTACAGGAATTCAACTTCCGTCGTTGTCAGC |
| | | TGGCGCGGGAACCCATCTTGTGTAAAAAACTGTATAACCAGACACGTGGACTCGACCGAGA |
| | | AACAGTCAGAACCTGTCACAAGAAATAATCTTGATTAAAGGCTTTGACGGCAAACGGACCT |
| | | CTTCCCTGCTGAAGTGTACGATTGAATATCCACATCGAAGGTCAATTACCCTCATCTTTTA |
| | | CATGGTCATAAGACAATAATCTCCTATTTGGATTAAAATCCGCGCACGAAAGATAAGAGTG |
| | | GAATCGATTGCATTATCGAGTTTTTAAGCCCCATACCCGACAGATGTGTAAAAAGTGTAGT |
| | | GGTAATGGCGTCACCAAGACCTATGCTTCTCATAATAATAGGACGTATGCCCTAGCTACTG |
| | | CTAACGGTCGCTCTTACAATACTAGCTAAAAGAAACAAATTTGAAAAGTTATGTAGGAAGT |
| | | CATTGGCGGTGAAAAAGTGAGAAAAAAGGTCCCCGGAGACTGTGCTTTCATGTTATCAAAG |
| | | TACATGCCGAGTGAAGAGTTTGTTTTGATCAACTTTTATTATCTGGAGTCATTATACGATA |
| | | TTGCCATGGTTCCTTGGCTGTCCAACCAGGGGTCTTTTACACCAGATAATCTTCTACTACA |
| | | CTACACCTCAGGTACGATTCTTTCGTTATCAATCGACTACAAGATTATAGTGTCTCTAAGG |
| | | CGTGATGTAGGTTTTCCCTCAATGACAAAGACTTTACAGCAATCCGGTTCAATACGAGAAT |
| | | TAAGTGTGCGAGTAACAGCAAAGTAAAATCTAACAGAAAGGAGACTCAGAAAACAACCTAT |
| | | TGAGGACTGTAATATCAACTCAGCATTATTGTTTACTTTAAAATCTAATAATCGTTTCGAG |
| | | GATATGAGCACGGTATCCTAACATCAAGACAAATACCACATCATCTAAATACAACTGGTTG |
| | | CAATGAGTCGAATCGCGAACAAATAAAGCAACTATAAGCACGATAAACCACTGTTATGGGA |
| | | ATGATAAACAGTCTTATGACGTGGTCTATCTGTCGTAGGTGGTAAAGCCTTCTGAAGATCA |
| | | CTATCCAGTTCTGGCCTCAAGAACCATTTAGACAGCCTTTTCTAAACATGATCGTTGCTAT |
| | | AAGGACCGGGGACACCTAGACAAACTCACGGAAGGGATAACTTACATC |
| 254 | 38.90% | ACTGCTTATATAGGAGGTACAAACAGATACAATCCTTAGTTAACTAGAGAGAATGCTTTTT |
| | | TTCGACCGACACGCTTATAACTTCACTGGGCATGGTCACCATATTTAGGTAAAACAAACTG |
| | | CTGCGCTATATGTCGTACACATCCTGAGTGTACCAATATGTAGGTGGAAGGCAAGTTCAAT |
| | | GAGACGTCAGTTACCAAGCAAATTTACATTCTAGCAGTTATAAATGTATTATGACGCAGTT |
| | | CTTGTGGTGAGCGATCATTTACATTAAAACTTTATTCAAGAGCGTATATTAGCATATATTT |
| | | TCCGGAGAGTGCACTACGGGCCGAAATTTAGGCTGGAACTCCGCAAATTGGTTACGACCCT |
| | | GTATACATAGTTCTTATTATTAAGTAAAATGTGTGAATAAAACCTACACGACGCGTGATAT |
| | | ACGTAAAAGTTTATCTCTTGTAGTAATCAACTAAATTAACTTACTACTATCTGGTCGTCCG |
| | | TATGACCCTGTGAGCAGATTATTTTCGACTCGACATCTATGAATTCTACGGCACGAAAAGT |
| | | TGGTAACTTGTACTGGGTTAAACAATGTGTATTCGGGAGTCTGCGGAAGAACGTTTTTAAT |
| | | GTAACTTCCTTTGCAAACCAAAATTTGGTCTATTCAAACTGACACTAGCGTAATCTATACC |
| | | GCATGAGATCCTGACATGATCCTATATCTATGCGCATAGGTACTCGCACCAATAAGTGGGT |
| | | CGTAGAATTTCACGTAACTCAATGTTGTCTCCTTTCATTTTTTGTTAATTCGAGAAAACTA |
| | | CAAAAATAGTTAGTAAAATGCTCAAGGAGTCAGGTGCTACCTGTGGAATACATCTATGTCC |
| | | AATGGAACTTGCTCCCTCGGATGTGCGATTTCGTTGTTCAGTTGGGCCTTTAAGGAATACA |
| | | GCAACTCCAACTCTTTGATTTTAGGTAAGTATTTGATTCGCGGAAAGTACAGTGTATAATC |
| | | TGTTATTTGCCAAGACGTCATCGAAATCGAGTGTATCGAGATCAGACCATCGCGCTATCGC |
| | | AAGATATGAAGAGCATAGACAGATCACGATGCCAATCAGTGTCGATGGTGCGAAGACGCAG |
| | | CCCCTGTGATCAAATCGTCCGTTTCTCGATTTACTAGCGGAAAACAAAAACGAAGCGGTGA |
| | | ATACCCTGCGAGCTAATGTCTTTACCCGGTTATACGAGCTGATAACTCGGAAAATGCTAAT |
| | | ATCGAGGCTGCGCACTTAAAAAAATACTTTAATAATATTAATAAGCATAGCTGTATCATAA |
| | | CTTAAAATTCTACTGTATGATTTAGAATCTAACAGTGTTAACGATCTACAGACCGCACTAA |
| | | GATGAAGACGGACTAATCTCCTCCCTAATTTTCCTTGTTGATTAGCAAAGGGAGATCCTTT |
| | | TGTTATTTGAGGTTTACGAGAAAGATGTAAGAGTCGAAATAATTACGTAAACCTCATAGTC |
| | | GTCACCTAGAGCAACTATAACATGAACCACTCGCCTTGGTTAAATATAAAATAACTTCTTC |
| | | TCTGTAACATTGTTGCACACAAGCGAGCGACAAAATTTCACAACATTTGTTGCGTAGATAA |
| | | TATTACTGCATCATTTTTGCGTCAGAGTGAATGTCACTTATATAACTAGGAAAAATTAGTA |
| | | GGATAGCTCTTGCGGTTGAGAGTAATGTCGACTGAATCGACCGCCATAGATGGTAGAGGGA |
| | | GTGATTCAAATAGATTAATGTATGCGCTCCATCTATAAGGACGGACAAGGATCAATGTTCC |
| | | CTTATACTTAGCTAACAGGACCCTCTCCGAAGGTCTGATAATGCACTCATATAAGCATCGA |
| | | TGCGTCCTGAGTAGAAAAATCTTTACAAACTTTTAATAGATAAGTTATCTTGGAGGTGCTA |
| | | TCTATTCAAATCTCTGAACAGATCTGCGGCATGATAATGTCTTTGTACCGGTGTGAATAAT |
| | | GTGAGTCAGACGTCTGTGCGAAGTGGGAACCGAAATCTTTTAATCATT |
| 255 | 40.90% | GATTCGGTCGCGTTCCATAATCGAACCCTTAAGCCCATCTTCCAGCTGTTAACGTTATGTA |
| | | CCATCTTACCTCAATGTCAGCGATCTATGAGGTTCATGTTTTTGGTGGATTAAAAAACTTC |
| | | TTTATAGTGGTTTAGACAGAACGTTTAGCGCTGCGCTCGAAGTGTCTTATCTAACGGAGGA |
| | | CTAAAATTACCTGGTCACTCCTTAGACTTTTCGTAGTACTTAATTGCCGGACATCCGTTGG |
| | | GCTACACCAGCAAGAACACAAAGTGGTATGTGTGAAGCTAGACTGACCTCATGATTCGTAC |
| | | TACATTATAAGAATCAAGCTTCCCGGATTTGTGTTCTGAGATATTACCACGTACATTTTTA |
| | | AGGGGGTTCTTGACATCGTAACGCTAAGGCTGATTAAAGAGGAGGGTGCTATGCAGAGTTT |
| | | ATTGGTGTTTCATCAATGTATCACACAAAATTAGCTACTATAGGAAGTAGCTTTGGTGCGA |
| | | GCAGGGGGCGGTATGGTTAAGAAAGCTATGGTAAGAAAGGCCCAGGTGATACTACGTGTAA |
| | | GGTTGTGAAGAGCCACAAGAGCCAAGTTTTGATATTCGACTTCCTCCGAATCTACAGCTTA |
| | | TCGAGGGTTAAACGTTACGCATATTACGAGATTACATGATAGCTTCTCAGTTCTAGCACAT |
| | | TTATGAGACCCTTTGAATGGTGTCAATAAATAGGAGGTCCCCATATGACAAGTAGAATACT |
| | | AACTATAAGAGATTTGTAACGCTGGATACCATTTGCAGAGGATTGGCCCAAAGAATGATTG |
| | | CCCAACGCTTATATTGTCAGACCTTGGATTAGAAGAATAACGCAGAATACGACTGCAGTTT |
| | | GATATAATTTTGGCTCTGGGTTGCCTTAGTATCATTACTAATAGACTTGTGGTCTATATCC |
| | | ATTTGTTTAATGGAATAGACTGGGTAAAACACACCTCTTCCAGGCTGTAGTTCTTCATGTT |
| | | GTAAGGATCCGTCATGGCGTGCAAACTAGGGGAGGTATTTTTTGCTAATTGCGGTAACGGC |
| | | TCCAGTTGGGATATCGTCAATATGTGCCACTCGGCCCTTTCTCTGAGACGCTAAGATTTCC |
| | | GTAAGGTATAGCGATAAGAGTCTCTAATGCCAGAGGAATTGTTACCGCGAGCAAGATTCAT |
| | | GTCTATATATAAAATATCATCCACTTTGAATTACTGGTTGGAATCATCGTTCGCGTTATAA |
| | | CAAAAAACCTTTTAATTATGTTACCACAGATCTCGAAGTCCCTTTTGAGGCAGAAGTTTAA |
| | | ATATAAGCTCTAATTGTCGCATCTAACGGGTATATCGTCTCAACGGTAGGTCAAAAACATT |
| | | TGTTAACTTCAGACTGTACATTCGCATTTAACTCGCCATGTAAACCGCAATACATCTCGTG |
| | | CCTATCTCTCCTAGTAACGTATTATCGCTGGGTGAAAGCGCAACTAAGTAATAAGTGAATG |
| | | TCATTCACAATACCTAACTCTATCCGACGCGTAAGAGCGACCCAGCAGTTTAATGACATGA |
| | | TAAATCAAATTCTATGGAAGGCAGTACTTGCTTTGTGGACGATAGCGATTTTCCACCGTAT |
| | | TGCGAAGTCAGTTATGCTGAAATTTTATTCCATTCGCATAACACCAAGGCTTACTCTTAGG |
| | | AAAAAATGTAATACCGATTTTGGTATGAAGTATGTTACAGTACAGAATGAAATGCCCGGCG |
| | | GCGTGGTCAAACTGTTTCCTGAGGTTCATATAGGGAAAGGTCATCCCTCAGAATTGGCCCC |
| | | GTAATCGCAAAGCCTACGGGAGCTTTCTTAAGTCCAACCGGTAAAGCCAAATCTCAATTCA |
| | | TATGAGGAAATGTTTGACCGATAAAGAATAGATTGTCGAACTAACAGTCACAGAGAAAATA |
| | | CGAGTAGCATCACCTAAACAAAGCAGGTAATAAAATAGACTAATGGAGATCATCGTATCGG |
| | | CTTATGACCTGCGTCCATTTAAAGGCAATGAATACATTACCGACTAGA |
| 256 | 40.80% | AGTTATGAGGTTGACTTCTCATATAACACTATCAACAATGATCATCTCTTGCGAAACAAGC |
| | | GCCCTACACAGCTTGAATGGAACCAAGAGCCATAATGAGGTAAGGGACGGCTAGTTACTAA |
| | | TAAAGGAATCGATTTTACAAACACTAAATGAAAAACTTGCGCTGGTTGCAATGCTATAAAA |
| | | AAATGAAATGCAAACCAGTGAAGATCCCGATCAACCGTTCGCTGATTTTTATTGATGCTGT |
| | | ACGTTGTGTTAGTTTAATGATATATAGGCCATCTCCAGGTTACTTAGGACGCCAAAATTAC |
| | | TATTTTGAAGCTCAACCGTGGTATAATAGCTACAATAATTAATTGATGCCTGCAGGTCGTA |
| | | TCTCGAACGATTGTACGCATTACCTATGATATGAACAGAATCTGTATCCCATACTTAAAAT |
| | | CTTGACCTTGTAAAGATTTCGCATACGCATTAAGAAATTTCGTTCTACCCGCACGGATTGT |
| | | CCAAGTATATCTGGCCATTCACAGAAGTTACTAATCTTCATCTCTAAGTTTAAGGCCGACA |
| | | AAGGGTCCAAAACCTGCGTAGGTTACAACGCAGCTTACACTCAGTGACTAACCAACGCTCA |
| | | GTAGGGTAACTGGACTTGTTCTCGCTATTCAGCTGGTACTGTAATGATCAACTTAGAACGG |
| | | CCCTATGGCTAAGCAAGGAGTACGCAATGTTTTAGAATACGTGTTTGCTCACACAGGTAGT |
| | | AGTTTAATATACCCCCTGACAAGATATGTTAACATAGATGAAGTTTGGTATTACTTATAGC |
| | | CAGACTATTCTTCAACATATACACTGGGTTTTAGGAGTGTGGAATTTATAAGGACAGTTAT |
| | | ATTCCTACAATCGTTGTATGATCCTTTTGGGTTTGGTAGAACTACGTTTGGGCCGCGCCTT |
| | | TGGTCAACCACGGACTTTCTGTCTAGATGCCAATTCCTACAAGCTTAGTCCTATCAATTTA |
| | | GTAGAGAACAAATTTTGTCATCACTGAATTGTCGTCTTACTATCGGATCATTCTCCGCTAA |
| | | TTATAGGATTATTAGTAACGCGTATATAGGAGCGATTAATGACTCATCAATGAATAGCATC |
| | | ACTAGGTGTATTATATGAACCTCTCTCTATTCTATTAACTGCCCACTGTGGGTAATTTGAG |
| | | TTATACCTGACCGGTCCCTCGGATCCTTAATCCTTTGATGTCGATAGGTAACTGAAGTGTA |
| | | AGATCCTGATATATGAAGCCGGTAAGGAGACGGAGATTTTATATTAGTGTTCTTGGATACT |
| | | GTGCTAGAAGGTTCTACTCTAACTCAAACAGGTTATAAAGTAGGAAGGAAAAAGTTGATAG |
| | | TGGTAAACTAATTATGAGTTGGCTTGCTTATTCCAAGTTAGCGAGGTTTTCATGACGTAAG |
| | | TCTGATAAGGTTTGCTGGAAGCTGAAAAGTTTTACAAAAACGTTGTTTTAGAATGGTTTGT |
| | | CCCCGAAAATCGAACCTGGGATAGCCCTCAGGAGACGAACAAGCCCAGGCAAACCGGGGGT |
| | | TTCTCGCTTATTGCTATAATCACCTCTAGTGTTGTAGAAGCAATTACGGTGGGGAGGCGTC |
| | | AATGTGGCCTGAGTTCCGTTGAGGACTTTTCACGTGTAGGACCCATTAATAGAGGAGATAT |
| | | ATGTCTTTCAGCTGCGGAATTCATAATAGTGGAAAGAAGAAAAGGGATTACTAGATTAATA |
| | | TTACTCATCCCAGACTTAAGTTGAAAGCTACATCTTCACACCCAGGAAACCGGACCGCCTT |
| | | TGTTCAGGTCTAAGTAGTCTGGAACAGAACCGTATCAACTGCCCCAATTCATAGGTGTTAG |
| | | CGTGACAGCGATCGCGGATTTTTAGTCCAGACTGGCTGGGCCATCCGCTTCAATAAGTTAG |
| | | AGGACTACATACAACGATGGACCGAATTGGCAATAGTCGTGGTAAACTTCGAAGGGGCGGT |
| | | GTAAGATTCAAGCTGTAGTCGTGATGAAGGAGATCATCGTATAAACAG |
| 257 | 39.70% | ATACATCTAGACTACTAAGAGGGATTATCCCAGCGCAGTCCCACCCAAACATCAATCTGTC |
| | | CCTTTGTTCTAATATATCTCTGGTCGCGAATGAGTAAACGGGGCTAAAGGTCCATTATTTT |
| | | TATGTAGGAGCATGTTGCTTATTATGGCATAGCAGTCGCCATCCCCCTGTCACTCGATCTA |
| | | GATACATCTCACATTGATTGGAAACTTCTACAAAACGTTAGTACTTAAGATGAGTGATTTA |
| | | GTGCATTTCTCGTTTTCACAAACTTTGCTAAACAAACGTATTGAGTGGCGCGTTTTTTGAT |
| | | TTGTCGCATAACCGTTTACTCCCTGTTCGAAGGAAATCGATCTCCTTATAAATAATGAGTA |
| | | CATTATACAGCTAGCATAATCTGCGTGTGGCAAAAGTGAACGTTTAATCTACAATTGATGG |
| | | AAAAATAGCCCGTTAGTCCTTTTAAAGACGTCTTGGAAAAATATTGAGAGAACCTTCGTCC |
| | | AAAATATGTCAAAGCTTCGTCACATCTTTTCACCTATTACTAACTCCGTAGTTCAACTGAC |
| | | TTTAGAGGGCAAGTTTTGAGACAATATCTTAGGGCTGACTAATAAGACGGTTATATTTCAA |
| | | GAAGGAAAGATCTTAAGAGTCAAAAAAACGTCAGGGCTATCGTTACGATATTGGTATGAAC |
| | | AGTAATGATATATTTTGCAGATCTTAATATAACGACATTCGAACACAATAGCGTCAGACAA |
| | | AGGTTACCACTCCTCTATAATTACTGCAGCTTCAATTGATGAGCGTCATTTAATTTTGGCC |
| | | GGACATTTACATCGTGAGCTGGCAGCACGCTCAGCTTTATTGTTCTTGCCAGAACATTACG |
| | | AATAGCCGTTCAATGCCAATTAGTATGATAAAAGTAGTGAGTGTAAAACATGGCCTGGGTT |
| | | TAAAGAATGAGTAACTATTATTTTGTAGGAATAACTGATTCCCTTGAGTTCTATCTTAAGT |
| | | TGTACAGAATCACACTCCTACAGCGAATAAGCAACGACATAGAATCCGTTATTTCGTATGT |
| | | CTCGGCGGGACATGTATAAGTAGCATACGTTATATCGGTTGTCGCACGAACCGCCTTCATT |
| | | CCAAAGGCGCTTACAAATCTGCAGTAAAAAGCTTAGCATTTACTATAGAGTATCGGCGTTG |
| | | ACCGTTAAGCCCGTCCCGTCCATTCAATCACTCAATTGATCATCTTTTGGCAATAGTCGTC |
| | | ATATGAGAAAATAGCTCTGTCGTTGTTATTATTGGCTAGAGTATAAGCTGTTAAACTACAG |
| | | AATGACGTTTTGTGGAAAGTGGACGTAAGATCCTTGTTCGCGAAGACTCGCACGGTGGGGA |
| | | ACAATTCCTGGGAATATTTGATCTACGTACGGTTATTCTGCATGTGATTACAATATTTCCA |
| | | ACGCAGTCCTTTTGACATTATATGAAACCAGACCCGATGCATATGTTTTCTGACTGGTGGT |
| | | TTGAGTCAGAGTCAACAAAAGTATCAGTCTTTCGTTACTAAATCTTCCTAAGTAAATGGTG |
| | | GGCGACCATTCCTTGTAACCTGTTCTGTTATAGGTACTATTCCAGCCTGGAAATCGTGGAA |
| | | CACATCGATCTAGTTGTCTATCTATAAGAGAACACTCGGTTCCAAATATGTAATCCGCACG |
| | | TAAGAGAGGAGTCTCGTACATGATATATAACGTTGGGTACATTTCTTAGACATTCCGGTGA |
| | | TACATAATGTACAAGTCACATGATTACACCAGCTGGTAGATAGAATACCTGAGACTGGGTC |
| | | CTAGATGATTATAAGAAGTGTTACATGGACGCTCTCGTTTTGTTGTTGGCTTAACACCAGG |
| | | GCTTGCTCCATGTTCTCATGTCGTTATTACTGAATTATCTTCCATTATGATCCTGGACGGA |
| | | TGAACGAAGCAGAAGATAACAAAGATGACTGAATGCCGGAAAAGGAATTAGGCCCTGATAT |
| | | ATCGCGCTTCTTTATGCATGTTTAGGCTGTACCAATAAACGCAAGAGG |
| 258 | 40.80% | GTACCCGTATATCGTCACTTCATTTGAAGCTATTATTAATGTAAAATCCTTCCGTCACACA |
| | | CTCTTTTCAAAAAGGGAAGTCTAAATTAACATTCAGATGAAAAGCGCTGACCCACATGGGA |
| | | ATATCCTTTCTAGGCTATCAGCCGAAAAGCTCCAGCGATTAGCTAAATATCTAAGCCTCCA |
| | | GAACAGAGTTATTATATATTGGTTCGAATATGCTAATATTACAGTAGAAAGTAAGGTACCG |
| | | GCACTTTTAACGCCGAAGTCGACCGGTGTAGCTGTGAAAATATATTTAGTACACGTAATAT |
| | | TAATTGGAAATTGATGAGATCGAATCTTCAGGAGAATCTGACGAGCATTACTAATCGCGCG |
| | | TGACGGGAACGTTAATATACAAGCGTCTATTCTAGGTTATAATAAACTCCTATCTGGGAAG |
| | | TTGAATGGTTTTTTCAAAACTTTAACGTTCTGGCTATACAAAGCTAGTTGCTTTAACTTAT |
| | | CGCATACTATGATCCTTCCCATCAATCAATCTCAGTGACTATAAACGCAAGTGACACAATT |
| | | GTCTGCGTTCCACATTTCTAAATCTCTTATCGCTCATTCCCTCTACACAAAGTTCGATTAC |
| | | GAAACGCGGGTCTACACACAAGCTTACAAGGATTACAATATCCAATTTTTTGTTATCAAAG |
| | | GCGAACTCAACGAATTTAATCGTTGGTCATTGGTATGGAATGGCGATTATAAGAAAACTCT |
| | | TTTAGTCATAGTAGCTCGAGATGAAGTGAACCGGGCCAGTCGGTAGTTTCACTATCGCGCA |
| | | GTAGTCACGATCAGTTCTTAGAATCTATCTCCTAATCAAGTCCAACAAGCAATCCGAAATG |
| | | TTGCTTTCTATAAAGGGTATGTGTACCTGCCAATATTAAACTTGATTCACTCAATAGTGAT |
| | | TTTAAATATGTCCATATTTATGCAAGAATCATTGACATTAGTAAATTCAGCCGTGCATTTG |
| | | ACACAATAAAGGTAGATTTAGACTGCATATTTCCCGCATATTTATTATTGTCAACGCACAA |
| | | AGTTGATGGACCGACCACGATCGCATCGAAGACCGTCTAAACGACGATATTCTTCGGAGAT |
| | | CCATATTTGTTTTCAATTACCGACCATTGTTCATCAAGTGTAGTTCAGTCGGAAATTTTTC |
| | | GTGTGCTTTTTAAAATACCAAATCTGAGGAAAAAGCTCGCTAGATGTTGAGTCAATCCGTA |
| | | AGAATATGCCCCAGGAGACATATGTAAGTCACAGCCGTAGACTCTCGGTTACCCCACGATA |
| | | TGTTCCATATGCAACGTTTGTTGAGTAATATGCAGTTCAGTCGGGCGTATTATGAACAGAC |
| | | AGACTGGCACAGTAAATTTTATCATCGGGTTTAAAATATCTAGATACCTCAGTTTCAAGGG |
| | | GGAGTTGAACTTTAACACGAGATCAAACTACATACACAAGATTATCAGTGGGTACGCTGAG |
| | | ACTTATCCTTAGCCTGGAGAGAGTCCAGCTACAGGAACTGCTAGTACTTAGCGTGCGACCT |
| | | CAAATCGAGAGAACTAATTACCCTGATCGACAGATCGGGCAAGTTAAGCAAACGCGGCTCG |
| | | CGTGTAGAACCATAACAATTGGAGATGCTCCTGCTTAAGAGATTATAGAACCGCAACCCAT |
| | | CAATCGTCAGTTACCCGAGGGCTCACGCACGCGGTGATGGAAGTTAGTTCCTTTGTACGCA |
| | | CGAGCTGCAATACGTGGTGATTATAATCGGCGCACACTAAAGGGGTGGATAGAATAGTAGA |
| | | AGCATATACGTCGCATAGGCGTACGCGGGCGAAAATTTTAATCGTTAACGTGGCACTAACA |
| | | GCGTTTTGTCTCCCCACTCGTGGGTTGCGGTGCATCGCACATATTCCCACAACACCTCTTA |
| | | ATGCTTTATTATTTGTATTAATGGCGGGAATCTGCCTGATATTAGTATTCGCACTAGTGGG |
| | | TAACGAAATCTTAGTCGCTGGCTACTGCAGAACTAATTGCGTTGCGAT |
| 259 | 40.80% | ACTAGCTACAGATCTGTAATAGAAAAATGCAGATGCTTGTTCTGCGTCGACTCGCTCATCA |
| | | ACATCCTGTCTCACAAGTTATGCATCCTGTGCATTTTATTGAAGCTTTGATGGGGATTAGA |
| | | TCGTGTATGGAAATGTTTATTCGCCTGGATAAGATCTGTCGGCTTATTCGTGGCCAATAAT |
| | | AGGTCAATTTGCGGAAACATAAAGACTCGCATACCAATACTCGCTTATCCTGAGGTTAAAT |
| | | TTAGTGTATGTAGACGAACAACAGTATTTAGTAGTATGACGTTCCCCCGTATTGCCAGAAC |
| | | TCCTGAATATTTGGATATGAGGTATGACTACGAAAAAAATACTACGTTGCTCATAACGATT |
| | | GGTGCAGGGATACCGAACTCATTGTTAAGGGACGCCACAGTCCAGTCTCTTTTCGTTCAGA |
| | | GCGTGTTTTTCAAAGTGCTTGTATTAGTGTGGACAGAGTTTACTGATCTCTCCGCACTTGG |
| | | ACTGATTGTGATCCCGATCATCTCTTTTCATAATTGTAACACGCTTTCATAGTACACTTCT |
| | | GTAGATTGAAGAGTGCTTGCAGCCGGACAGTCCTATAGAATTTGGCGTTTGTTCGGCCAAT |
| | | GTGTGCATTTTAACTTTAGGCGCCATCTCTTGAGATTACTCCTTTGAAAAATTTTGGCGGA |
| | | GGTTAACTCTGGTCTTTAACATAGGCGTGCTTAACACGAGCTTTACGGTCAGGTACAGGTA |
| | | ACAAAACAGGTCTAAATTTATTTAAGCAGCTTCTGATACTTTCCAAGGGTCACAGTTGGGG |
| | | AGCCTTCCGAGGTATGACAATCAGTTTTCAAAAGGTGTAGAATATCATATATTCTATCTAG |
| | | GCCAGAGCATTCTAAGCTGTTAAAAGAGTGCTATGCTCAGAAGTTGACTGTTCTAATCGAA |
| | | AATCGGACATAGATAACCCGCATACCACAAGTCCCGTTGTAACGTACCCATCGTTTTTGAT |
| | | TCTATGTCTTTGCTAATGATTGGCGATTGAGACATCCTACTTCTGTAGCTTGGCTGTTATG |
| | | CGATCCAAAATGGTATCCAGTGGTGGATGTCCGCCGCAAACTGAAACTCCCTATCAGTTCT |
| | | TTGAAATTAATTTGCGGGCTATCCGACTCATTCTTTAGGAATTAACAGAAGAACACGCGTC |
| | | TGTACCAAGGTTCTTCTTTGTTATATCACATAACAATGAATCACGTTCTATGATGTATCCA |
| | | GGTATAGAAGTTGTAGGTAAGCACTTGTATAAGGGGGCGCTCCTCTCAGATTGATTCATTA |
| | | TTTACTAAAAAAGGAGCGTGTTATTACTTCTAACAACTCCTCGCCATTATATATTATTTAA |
| | | CTACCATTCCCACTAGAAATGGATATCGTGTTCTAAGACCCTAATTGTGCTCATTAAACTA |
| | | ACTACCGCACCAACCGCCTTGAATCACCGGACCACACTAGTTAAGCTGCCGATACCCAATA |
| | | TGGTATTTTAGTGTATACCGGATATGACCTTATTTACGAATGGATTGAGCTCACCCCATAG |
| | | ATCAGTACCAGCGTTATTATGAAAATCTTGTTATTTTAACAGAGAGACATGCTTGGTCATT |
| | | ACTACGAATTTGAGTTTACGTTATACAAGGCGATCCAAACGGACAATAGCGCGATACGAGA |
| | | TTATAGTACCAATAGCACGAATCAGTTTTAGCGATCTCGTCCGATCTGTCAAGCCGAATGA |
| | | CTCTGAAACGTTAGTATCTGAAACGTTTCATTCAGCCTAAGATATGTATAGTATCATTATA |
| | | CCGTGTGGGTAGAACAATCAAATGCAGATAAAGCTATTTAATGCACTTCACATAACCTCTC |
| | | CGTTGGAAATCCATGTATTCTCTAATCAATTGAATTGTACCTTAGAAAGCACAGGGGGACA |
| | | CCTGAAGACCTCCCATCTCTTAAGGTTACCGGCACGTGAAACTTCAAAAGTCAGACAATCA |
| | | AACGGCAACGTGAATGTCTTCGGAAGTGGTGGTATGCACATCGCGTCA |
| 260 | 41.70% | TTAATAGAAGTAATAAGTGCTATTGGACTAAAATCGCGTCAATTAGCTATAGAACAGCTCT |
| | | GTGACGAACTATCAATGGGGCATTCGTTCACTAGTGGATACCGTACAAGCTCGCCGTGATC |
| | | GTGCGTCAAGGATAGTGCCAGAGCGCCGCGCTATATGTGTAACGACGCATAAGTAGATGTT |
| | | TATGTTATTGGGCAAAGTCATTCTTATCCATAATAAGCGCTGCCGATAAAGATTCATCAGA |
| | | GATATTGAGATTCTCCATACTTGACTAATCTCTGAGTAATTAAAATATATTTCTAATCGGA |
| | | TAAGTTAGGGATCACCGAACCCAATGAACTTAGTTTAATGTGTTCTCGCGAATATCCCCAT |
| | | GATATAAAGATCCGAATACCTCAGCTCCGTGCGTGCTCGTGCAGTCGTGCGTTTTCTATGA |
| | | ATCAACCATCAGTAACGAGTAGCGGTAACTACTTCTCGAGTTTAACCAAAGCCTATGTATA |
| | | CTAGCGTGCAATCACGTGCGGAAGGTCCGACCTACAGCAGCATTTTCGTTCGAAAAACGAA |
| | | AACTAATGTGCACTATGTTGAATGGGCATTCAGGCCTTAACTTCTAACGTTAAACTAGATT |
| | | TGCGATTATTAGGTATGAGATCGACCAGGTCGCCACAGATAATTAAAGATAGCCCTAGCAA |
| | | AGTGATAAGGTCCGGATGTTAGAACTTGCAAGAGTGTGTAAGATTATTTACTCTCGGTGCG |
| | | TCGACAGGCGAAACCCATAACTTTTATCGGTCAAGATTACGACCTTCAGCTAGTATCTTGA |
| | | GATTTGAAAGGGCCTAAAAGCAATTTAGTGTACTTGTGTAACATAACCTTAATTATTGATG |
| | | GTTCTATCGACTCCCAGCGGTAATAATCTTGTAATATTGTCGGATTTAGTTGAAGGGCAGG |
| | | TTGACATACCGAACAATAGCTAGTATCAATGTATAACTAGGAGGCATCTAATTTCGTAAAC |
| | | ACTCCTGACACTTGTCGTGTCTAAGCATGTTAGGACAAAAGACCAGTTTTTTTAAACCTGA |
| | | CTGTACCGGCAACGCCACAGATTTTATGTCTCGCATACGTACGAACTGAATTTGAGGGGGC |
| | | TCAGGTTTGGACTTACACCGCACGTGACTATACTGAGATCGAGGCTCCATTAACGGCAACA |
| | | TAAGACTAGCACTGTATGATCTGAAGCCAGGCTCTGGTGAAATTGCGGGTAGTTAACGACA |
| | | TTTATCGACGAACCCTTGATAAAAAGTGATTATGTTGTATCTGCGTGATATATTCTTTTCG |
| | | TGTTCAGTCTCTAGAACTTCGTGCGTAATAAAGATTATAGAGGAACGGTTAACCTCATTAC |
| | | AAGACGGAGACCGTTCATAGACGCCGATGGATTACAGGGTCTACTATAGCTACCTAGAACA |
| | | CTGGTGAACATAGGGATAACATACAATTAACAATATTCCGAGCCAAATTATGTCTTGAGTC |
| | | TTGGTTGTTATCTATATCGTTATTATGTTAGAAACTAATAAATGCGATAAGAACTAGATTT |
| | | TACAGTAGATCCAAATACCGGAATCTATCGGGACGATTGATTAAGACTTACTCAAACCTAA |
| | | CTTTAGCCCGATTTTGCAATTAGAGATACGTCGATTTCGAGACAAGAGTAGCGTCCCCATG |
| | | GCAAATATCCACGGACAGATAATGACACGTGAGGGATGGCAAGAGTAGTTGCTCAGGATGT |
| | | AGGCGTTGATGGTCTGGCGCTAATGTCGTGGCTACCTGTTGAGTCTCGCGTAATGACTAGT |
| | | AGTGTTCGAACGTATGACCAAGTTCCTTCCTAGTGTTACCACTTTGACACATACCCAGGGG |
| | | TTTGCCGCATGTCGCTACTATAGTATAGGTGCTGCTATGAAGCTTCTGAATCAGCGGCTAA |
| | | CAAGTACCTAAGAAAATTGGACATCTTTTGGATGACAGTGCACAGGAGCCTATACTGAATT |
| | | ATCGGTGATCGATGCTTCATGTAATCAAAACCAGCGCGTACACACTTT |
| 261 | 39.10% | TAGTCTTAATTCATTACATATTGTGCGGTCGAATTGAGGGAGCCGATAATGCGGTTACAAT |
| | | AATTCCTATACTTAAATATACAAAGATTTAAAATTTCAAAAAATGGTTAGCAGCATCGTTA |
| | | GTGCGTATACATGAAGAGGCACGTGCCCCGGAGAGAGGAAGTAAGCTCTTTAAAGATGCTT |
| | | TGACATACGATTTTTAATAAAACATGAGCATTTGAATAAAAACGACTTCCTCATACTGTAA |
| | | ACATCACGCATGCACATTAGACAATAATCCAGTAACGAAACGGCTTCAGTCGTAATCGCCC |
| | | ATATAGTTGGCTACAGAATGTTGGATAGAGAACTTAAGTACGCTAAGGCGGCGTATTTTCT |
| | | TAATATTTAGGGGTATTGCCGCAGTCATTACAGATAACCGCCTATGCGGCCATGCCAGGAT |
| | | TATAGATAACTTTTTAACATTAGCCGCAGAGGTGGGAGTAGCACGTAATATCAGCACATAA |
| | | CGTGTCAGTCAGCATATTACGGAATAATCCTATCGTTATGAGATCTCCCCTGTCATATCAC |
| | | AACATGTTTCGATGTTCCAAAACCGGGAACATTTTGGATCGGTTAAATGATTGTACATCAT |
| | | TTGTTGCAGACCTTAGGAACATCCATCATCCGCCGCCCTTCATCTCTCAAAGTTATCGCTT |
| | | GTAAATGTATCACAACTAGTATGGTGTAAAATATAGTACCCGATAGACTCGATTTAGGCTG |
| | | TGAGGTTAGTAACTCTAACTTGTGCTTTCGACACAGATCCTCGTTTCATGCAAATTTAATT |
| | | TTGCTGGCTAGATATATCAATCGTTCGATTATTCAGAGTTTTGGTGAGGAGCCCCCTCAGA |
| | | TGGGAGCATTTTCACTACTTTAAAGAATAACGTATTTTTCGCCCTGTCCCTTAGTGACTTA |
| | | AAAAGAATGGGGGCTAGTGCTTAGAGCTGGTAGGGCTTTTTGGTTCTATCTGTTAAGCGAA |
| | | TAAGCTGTCACCTAAGCAAATTAATGCTTTCATTGTACCCCGGAACTTTAAATCTATGAAC |
| | | AATCGCAACAAATTGTCCAAAGGCAACAATACGACACAGTTAGAGGCCATCGGCGCAGGTA |
| | | CACTCTATCCACGCCTATCAGAATGTCACCTGGTTAATGGTCAATTTAGGTGGCTGGAGGC |
| | | ACATGTGAAGCAATATGGTCTAGGGAAAGATATCGGTTTACTTAGATTTTATAGTTCCGGA |
| | | TCCAACTTAAATAATATAGGTATTAAAGAGCAGTATCAAGAGGGTTTCTTCCCAAGGAATC |
| | | TTGCGATTTTCATACACAGCTTTAACAAATTTCACTAGACGCACCTTCATTTTGTCGTCTC |
| | | GTTGTATATGAGTCCGGGGTAAGAATTTTTTACCGTATTTAACATGATCAACGGGTACTAA |
| | | AGCAATGTCATTTCTAAACACAGTAGGTAAAGGACACGTCATCTTATTTTAAAGAATGTCA |
| | | GAAATGAGGGAGACTAGATCGATATTACGTGTTTTTTGAGTCAAAGACGGCCGTAAAATAA |
| | | TCAAGCAGTCTTTCTACCTGTACTTGTCGCTACCTAGAATCTTTAATTTATCCATGTCAAG |
| | | GAGGATGCCCATCTGAAACAATACCTGTTGCTAGATCGTCTAACAACGGCATCTTGTCGTC |
| | | CATGCGGGGTTGTTCTTGTACGTATCAGCGTCGGTTATATGTAAAAATAATGTTTTACTAC |
| | | TATGCCATCTGTCCCGTATTCTTAAGCATGACTAATATTAAAAGCCGCCTATATATCGAGA |
| | | ACGACTAGCATTGGAATTTAAAATTGCTTCCAAGCTATGATGATGTGAGCTCTCACATTGT |
| | | GGTAGTATAAACTATGGTTAGCCACGACTCGTTCGGACAAGTAGTAATATCTGTTGGTAAT |
| | | AGTCGGGTTACCGCGAAATATTTGAAATTGATATTAAGAAGCAATGATTTGTACATAAGTA |
| | | TACCTGTAATGAATTCCTGCGTTAGCAGCTTAGTATCCATTATTAGAG |
| 262 | 40.90% | GGCCCTATAGATTTTAACCTAAGCTCTAGCTTGTGTGTGCTCAGAGTACTGCTCATAAATA |
| | | TGCTCGATAAAGGAGGTAAGGCATATCGTAATTTGGAAGATAATACCACACTTATTGGTAA |
| | | CACGTTGGAATCACATATTAATTATGACCCAGCCTTGGCATTCGAGCAGGGATATGTGGGA |
| | | GTATCAGTTGAGTTTGGCTCCTTGCTACTGCCCTCTGATGCTCTGCTTGCTCTAGCTTAGG |
| | | TCATTAATGATAAAAAAGAGCCAGAGTGTGGGCTAAACAGGCAACGGTACCGTTGTAGAGC |
| | | GAGGTATTGCTATCGGGAGACGTCGGGTCAAAGTGGGATTCATGCAGTAAGTTTGCCAAAG |
| | | GGTCTGCTTAAAGAGACCGATTCCGGAAGGCTATATGCCATAGCAAGGTATGCACTGCATT |
| | | GAGCTGAAAACTCTTGAGCATAGTATTTACTAAATAAAGAATCTGATATCTTCTAGCGTGT |
| | | TCACTGGACTATTATTTAGATGGTCGCCAACAACAAGCGTGCGAATCATATAGACCCAACC |
| | | CAGGGTGGTATTGAATTCTATATTAAAATGTCTCGCCCTTATAACTCTCTAGGTTTCCATA |
| | | GTACAAACCTAGGTGTCGTCAACTGCATGCACTGCTTTTTGTATCGGTAATGTTGATCGAC |
| | | CCGATGGGCTTTTTTTAATAAAGGTCTTGTTTAGTTGATCATACTACCAATTTTGGTGGTC |
| | | GATGGCTCAATGACCAATGGAATCTTTATAGTAAAAGAGCCCTTGGCACCAACGAATCATG |
| | | GAATTTAGGACGATGTCTCATTTACCATATTTTGCATTCAGACTATGACTTTCAATAATAG |
| | | AATATCATCGTCAAACACCGTGGATATGGCATCGACAAGTGTTGGGATGCCCACTGAATAA |
| | | CGTCTCTTCGTCATCTTTAGGGCGGCTATCCATTAAGGAGGATTTTATTTTTATAGCAGTC |
| | | TTAGTCCGAGGCATTGGCGCCAAACATCGGCTCAACACTAGACACGTCTTTAATGGAAAGT |
| | | ATCTAGTGTTACTGCGGTACGGAAAGCAAGTTCAGTACTTTTATCCAATCTAAGTATCACC |
| | | CAGCTTATATTTAAAAGCTAGGTAATAGGGAAGTTACTAATAACTCATGCGCGTGTAGTGT |
| | | AGTCTTGCTGTCGCTTAAAGCAACTGAATGAATGTACGGCTGACAAAGGCTTACCCAAGAA |
| | | AACTCTCTTGTACGCTACAAGAAACCTGTAACAAGAGAAAAATATTTTAGCCCACGTATAG |
| | | TGAGGCCAAACTTGATGCCCGTAAAAGCAAACAAGTAATATTCAGCAGAATTTGCGGTCAT |
| | | TCAAGTGTTTAGGTACGTAACTTTTACAGAATTAGCTGTTGATTAGGTAATACTAAATCAA |
| | | AATGTCGTAATACCGAAGCAGAAGTATATGATCTAATTTGTCGCCTCGCTTCATGCTACGA |
| | | ATGTTACTTCGTTTATTACAGCTGCAAACTTGCAGTGACTTGCATTTGATAGGATTCTTCC |
| | | TAGGGAACCATACTGGGCCGCGGACAGGGAGTCAGGAACTCATAACGGATGAAGATGTAAT |
| | | CTCTATAGGGGTGAATAACAGGATTGAAGATAGTAATCTAAGTACTCTCATCTCGTGGACG |
| | | ACTTTAAGCGCACTGACAGCGACTCGCGATTCGACGAACACCCGTGATCGATTTACACGTT |
| | | CATTCTGAAAGATATACAGGTAATAATTCTAAAAGATAATTGAGTACCAATATATAGGTTT |
| | | TATGATCTTAGGCGCATGTCACTGACGAGAGAAAAGATAGTCTTGCCGCCTCTAAGTGTTC |
| | | TATTTCTGGACGTGCCTGGGCATTAAGGGCGACGTTGACTTTTATACACATTTCATGTCCA |
| | | CTAACAATTTTATATCACGTAGCAGGACATAAAGGGAGGACTCTATAAAAAGTTTCGCTAT |
| | | ATACGTACAGTACGTTCAAAATCTCCAGAGGAAAGCTTGTAAAAAAAG |
| 263 | 40.40% | CGCTCGACACGAGTATAACAAATATCGATAGATGCTATAGTGATAAGGTATAAGTAAAATA |
| | | GTACTGCGAATACAAATAGCTTGGAGAAATACGTTCATCCTTTAACTTCAAAAATTTTTGG |
| | | ACCTCAGGCACGTTGTCATTATTACTGGCAGGTGATACCACCCAAAAATCGTACCCGCAAT |
| | | ATATCTTCGGTAATTCTTGCCAAGTTGGGATTTTACATACTTAGTATTAATAGTGGGATCA |
| | | GCTTCGATCGAAGACCATAACTCAGTATGTGTATTCCTCATACAAGATTTCTGAAGGACGA |
| | | AGGCTCATCAATGCTGAGGTGTTATCAGGTCAATAACAAGCCGCATTAACGCCGTAACCCT |
| | | AATGCCATAATTCTTTGACGAAATGCCAAATAGTTTCATCAGGAATCACATTATTTGGATA |
| | | AGGAAGCACAACAAACGCTTTAATCTATACCCCTAGAATTAAGAGGACAGCATGATAGGCT |
| | | TTGCAATGAACCAGTCTCCTAAGCGTACCACCACTCCGGAGCCTTATGGCGCGCCGGTATT |
| | | ATGGCGATGCACTGCCTGGGCGAAACTCGAGTGAATCATTTTTCCCGATATACACAGCAGT |
| | | ACGCCGACGGTCTGGTAAAAAAAACGTTATAGGCTTTGACCGCATGGTGATCGTGGTTAAG |
| | | TGCCTTTACCTAGAGTGCTGCTAGATGTAACACAATTGATCTGACAGTTTACGACCTTGTA |
| | | ATCCAAGAACCATATAGATGAGCCGCTGAGTTAGTAAGATAATGCACGCTCCGGGGCTAAA |
| | | TCTAGTGCGGTTCATGAATACCGAATCAACTACGGTTATTGGCTGCGGTAGAATATTTAGT |
| | | TGTGTTAAATATACTCTAAGATGAACATGTATCACTATAATCACTCACCCCCTCTGCGTTC |
| | | ATAAGTAAGTGGCTAGTGTGATAGTAACTTGTATCAGCGACCACTACTATATGTGGAAGCT |
| | | TTTGAATGAGAATCTCCGCACATGATGATGTATTGATACAATTCTTTTGTTCGAAAAAGCT |
| | | TCGGTGTTTTTTAGGACAGGAGATTAACGCTTTAGAGTCATACATATATGTCAAGAAACCG |
| | | GGGAAAAAATGCCAGCCCAGAGTGTTCTAAACGATAGGTTGTTCAGTTTTTAATAACCCGC |
| | | GACGCGTCAAGTAACGTCACGGGTCAGCTACGATTACCAATTTGCTATAAACTTTCCCCCG |
| | | ACGAGCCAAATCCCTCAAAGCTGCCAGATAAAAGGATAGCAACCTGTACTCCCCGTCAAAT |
| | | CTAATGCATTCTTGTTTTTTAAGTCTCGTGTAACATGCGTTGGCTAATCTTCTCTACCGGG |
| | | TCCAGTGCCCTTTCAGCTTATGCCTCACCTTTGATTAGTAATGGACATCAGCTTTTAGTCA |
| | | CATCGGAGTGCCAATTATACCGTTATATCTTTCTCTGATGCAGACCGACCTGTCGTGTACC |
| | | GATTCATCCTAGGGTAACTAGCCGTGGCAAAATATCTTTATCGTGTTGTCAGGACTTGGTT |
| | | GTTATATACTCTAGCCCGTAGATTTAAAATAAATTAAGTGTAGATCGTCCAAATATCTAAA |
| | | GCAATCGCAGTTTTTATCACATCATGTGTTAAAATGCGATCAAAAGAAAAATACTGTTATT |
| | | TCGAGAGTCAAGGCTGTGAGGAAATATGATGAAGACTGCCATCCTGGTGGACTGGCGGCCC |
| | | CAACGTTGAAGTTTCTATTTGATCGGTTATTAAAGGATACTCGAGAACAACATCGAAGGAA |
| | | TAAACTTTTATAGAAAGTCTCCGAAATGAATAACTTAAGATATAAATTTATCGCGCGATAG |
| | | TTCTGGTGGATGATAGCTTTATTCCTCTTAATGCAGTATAGCTATTGCACCTATTAATTTG |
| | | TATAATAACGTATCATGTTAGACGGTCAGCATGATATTCCGGATAGTGGAAGCAAATTAGG |
| | | ACATCTAAATATGTCGCTAGTATTTGAGTCATTATAGCTTCGAGGCTT |
| 264 | 42.10% | CTCTAACGTGCATTTCTTCGTCGCCTTTGTAAGACCCCACAAAAACATGACGCTTTAGGGA |
| | | TATGGTCCAAGACTCCGAATTGAAAGTATGCTGGTATGATATGGGACGTTTTTGAAACCCC |
| | | CCTCTCACGCGGGTAATTGGGTTTTTAGTTAGTGTATCATAGTAGGTATATCTACGAACTA |
| | | CGTCTGACTGAGAGAGACTTTGTGCCTCTCAACCGCTATGGTGTCAGCGACTGATATTGGA |
| | | GTTATTTACCCGTCGTTATACGTGGGTAATCTTTACTACGGTTCAAGGTAACTAATCTAGT |
| | | GTAGGTAGAATGCTGAAGAATTACCCGTTGGACCCGGTAGTCCGTCCGCTCCACGCATGGA |
| | | ATGCATGAGTAACGTCTAGGTGAATATCCGGAGTGCATAACTTTTTGGTATCTAGTCCGCT |
| | | AGTGGATGGAGAATGAGATATTTTTTTGGAGTGGTTAGTATTAGTCTTCTCAAAGAGAACG |
| | | ATCATTATGTTGCTTAAATTCACGCTATGTTCTCGATGTAAAACAATTTTCGTAGAGAAAG |
| | | ATGCGTAAAACGCAGAGTTAGCATATAAAAAGTACAATCAAGCCCGAAGCACTCACAAGAA |
| | | ACATAGGGGCTAAATGTTACCGTCCAAGTGAGTAGGATTTAATATGAAGCCGGGCTTATTG |
| | | GGTACAGTACGTGGACGGACTACGACGCATGTGTGTTATAGAATGAAGTGCCTACAACTGA |
| | | AGCACAATTACTAAAGGAATGTACCTGGGTTTACACTAAGCATCCCATCCTCTTCGCGGTT |
| | | CAGCCTGATGTAAACGTAAATCTCGTCTTCCCATTATTAAGACGCCTCGATCTACGATAGG |
| | | TGATACGTGTACATCGGTGGACCATGTGTTTTGATATTCAACGATGTAAGTATGGTTCCCT |
| | | GCAGTGAACCCCTCTTCAAGTCGTCGATGTACCTGCAAGTGTACAATCGGAAGACCATGGG |
| | | TCCATATGTAAAAATAAGTTAGGGGTCTTTTGGTCTGTGTTGGTTATAATCGATATTGCCA |
| | | AAATATTATGGACAGTTAGTTCGAATTTTGTGTATGGTAGCCGTCGAAAAGGGTGGACGTT |
| | | AAGTATATCCATCCCAGCGGCTGGGAGATATGTAGACCGACGAGTGTTAAGTTATTCCACT |
| | | TAGTTTAGGACGAAATCAATACGATTATTTTACATCGGAGGAGATGACAACAAAAAACTAC |
| | | TCGGTTTCGACAGGTGGAAGATGTCGCTGCGCACCAGTAGAGCTTAGGAGAGCGACGGTAC |
| | | TCATTTGCAGCATGGGTACGTAATCACGTTAGTAAATAAGTAAGTATGCCTTCTCTTATGT |
| | | CATTTTATAAGCTATAATGGTGTTGTGCCAACTTAAAGATTGACACATGATATGCTACCAG |
| | | ATAAGCCTCGAGTCGCCTATATTTTGCTACTAAACCTGATTAACTAGAGAATAGGTATAAT |
| | | CCCTGGTAACCAGTAATTTTAATACTATGTTGCCACTTGATGTAGACCTGGCTGTGGTTAC |
| | | TAAGGTGCTTTGAAACCATTGACCACCCGTTTCTGCTCGGGTTGTGCATCTAACGTAAATA |
| | | TTCAGAGATAACGTGGCTCTGCTATTATTTTTATATTGCCTGCTGACATATCATCATCCTT |
| | | GAATGGCCAGCAACAGTTCTTGATCGGCAGAGGCCCCATGAACTAGGGTAATATAGCAGAT |
| | | TAACTATCGGTTAACTGTATTAAACTTGTGTAATACTTATATTGACTAATTGGGATTGCCT |
| | | TTGTCGTTATCTCGTTTATCTTGAAAACGGTGATGTTTTTAGAGGCGATAGTATTGAATAG |
| | | CTCGAATGATCACCAGCCATCAAGAATGTAGCTAACTCCGATACTCCTTGACGAGAGCTCA |
| | | AGCGAATACTAGGTCGGCGCTGCTATCCGCAGAGTTCAGGGTTCTACCCGGGGTATAAAAT |
| | | CCCATTGATCATTCAGATATTATGGACTTGGCGTTTATGCGACGAGTC |
| 265 | 39.60% | AAGAAGCAGCTAGTGCTACTTCGGAATAGTTGTCGTTTAAGTCCGTTCAAACATGACGCTC |
| | | TAGTCATTTTGAAACCTAAACCAGTAATAATAGACTGACTCAGAATGATTATACTGCTATC |
| | | TCTAGTTTAAGGAGATCCAGCGAAATAACTTGGTGAACTATGCCGAGATACTATAAAAAGA |
| | | TCAAGGACGGGTCGCTCACGGTTTTGGTTTATTTTACTACTTCTTCGTGGCTGTATTAGTC |
| | | GATGCAAGTTCTAATAAATAGCAAACGTTTTAAGTGGGATTAGTACATATTGATGGACGTC |
| | | CACCACGTGAAATCTCGCAGCGTCATAGAAGGAGCTATAACCATTCACTGCGACTACGACA |
| | | TGTGTTTGGGTAGTGCCAACTACCCGCTTCCGCGTCCCTGCCGTTCTGTACACTTATAAAA |
| | | TTGATATTTTAATCAGTGGATGTGCTGATACGGGGCACTGAGATGATGAATAGTATTAGGC |
| | | TGTAGTACCTTATGTACGCAAGAAATTTTAGAGTAAAGATTAGTCTGTGGGTAAGGAAAAA |
| | | GCTAAGTTATGATTATCCATGGCCATGGCATCTACAAGCTGATGAACGTACCAACATTATC |
| | | TAATTTAAGAACTTAACTTGTCTTATCCTCTCTTAAAGTCTTAATTTGCACTATTAAGCTT |
| | | AGGGAAGTCGCAACCAAACTCGTGTAGTATTGAGATAAATTATTAAACTTTCTTAGTATCT |
| | | ACTGATATCCGTATCAAGTATGCTTATAAATTCTTGTTCTGCCTGACAGGCTAGTGAATCC |
| | | TGCACCCGGGACGATTGCAGGTGTATACAGGCCCTCACGCTAGCAATCAATACCAATACGA |
| | | AATAAGGGCTAACATTTTTCGTAACAGATTAGAAGCAGTCCCGTTCAGAACTTACCACTGC |
| | | ACCAACGGAGGTACTGAATTCGGACTCATAGAATCCTCGAGTAGTAAGACCGTAGAAGAGA |
| | | CAGTGCATATTAATGTCATAGATCAATTTATATTTTATATGGTTGCCCATTTCATGATACC |
| | | CCTTTAAATTTATAACTTAGAAAAGGAGCCGCACTAATAATGAGCGGCATGCTGTAAAAAA |
| | | GTAGGCCAAAACGCAAGATAAGGTACCTTTGTTGTCCAATCAAATTAATTGATTTATTCTT |
| | | CGATCGATCGACCGTCATAGTTGAAGTAACTATTTAGTTACGGCAGATACAGCGTATCAAT |
| | | TCATTCGGTGACTTTGCTTAGATAACTGCTCGATAATCCGGAATTATCATCGTTCAAAGTC |
| | | CTTCCCTTACTAAGGCTCTTGGATTCAGATGATCGGTCATCCCTAACAAACAGCCCACTGC |
| | | CATGCTGCTATGGTGACATTCGTTACTACATTGATTTCTGCAGACCTTCATCCATAATACG |
| | | ATGGTAACGTCTCGCTTACTATGCACGGTGTGCCCCTGCCTATATCTTCACGATATACCAA |
| | | GTGGAGAACCGTAGGCATGTAGTCATTCAGGTGGCCACTCTCCTTCACATTATGTTTAGAG |
| | | GTCATGAATAACCCTAATCGTGTGACCTCAAACAGCATCGTATTCCGAATAAGTAACAAGT |
| | | AGGGGTGTTTCAAGTTGCATGACACAATAGGATATGATTCTCAACCAAACTTGGCAATAAA |
| | | CGCATAGGTTTAGCAGTACTAACAAGCCATTATGTTTAATATAGAGCATGGCTTACTCTGT |
| | | CATGTTCAAGGTGGCTAAACCCAACGCGTTAATACACTCATCGGTTACAGTGTTTTTAGAA |
| | | GAGCAATTGATATCTCTTCAGGTGATACCTGGTTCATTATCCTAATTCAGTTGGTTCAGGA |
| | | AGCCTTATAACTACCAATTCGATATTTTTAAGCATATAGATTAGGTGATACCACACCGTAG |
| | | GAAATTGTGCAGAATTTGGTGTCTAGAAATTTAACATTAAGTGATCAGAAAATTCTCTGTG |
| | | TTAAACGACTGTTGCGAATCTGTGTCTTTCAACCTCAAGTACGATCTC |
| 266 | 40.10% | TAACAACCTGTAACTGTCAACTAATGACCTCCTTACCAAAATTGAGGGTAGTTGGTTCAAA |
| | | GAGAATGCAGCATGACGCAGAGCTTGTAGTCACATCGTTCTTCTAGTACGCAGAGTGTAGA |
| | | GTTAAGATTATTAAACTCAGAGCACGTTGTGGACAAACCAATACCAGTCCATTCAATTACA |
| | | TGGTATCTAACAGTATCGTACAACTTTAATATGGTCTAGGGCTAGTGAAGTGTACCAACTA |
| | | CTTGATACGCAGTAAATAATTTCATCCTATCTTTACGTCGCCATCGAAAAGCAAAGTTATG |
| | | GCGCGTGGAAATTCAGATGAACCATAACCAAAGAGATAAATTGGCAGCAGTTTTTTGTAGA |
| | | CATTTATATAAGAAGAGCTCGAGGCGTAGGTTAATTCTATACAACGCTATGATAGTCAAGT |
| | | TCTACTTGACCAACTACGCTGGGAATGTTTATTAAATTCAACTGGGGGCAAACTAGCATAT |
| | | ACTGTCTGAGTGTCCTTCGATGGTTCTATACAAACGGGGTGTCGAGGTACTAGTGGAATGG |
| | | AGAAACTACCGACAAACGCATATCTTATCTTCTACTCGGGATTTATGAAATTTTTTGCGTA |
| | | TACTATTCCTGTGAGCAATGTTCAACAGCGTAGTGAGCCTCATAACGTCACATCAATTGTT |
| | | TCACGTCTGTGGCTATCGAGTATTCCTTAACTTAACTAGAGTATAGACATTAGAGTCTAAT |
| | | TCTATGCAAGTTAGATAACTACTACTACTGTCGTACTTCATTCAGTTCCTGCTCGTACTCG |
| | | GCGACGCTATAACCGGCCTAGTTTGTGCGTCGCCAGATAACTGTTCCTTTTAAACGTATAA |
| | | AAAGTACGAAAGATTAACCCAGCGGAAGTTGGGCCCCATAAATGTCATATAGGGACTGAGA |
| | | CTACTGTTAAAAACTCCTAGTATACATTGTAGATAATCAACTAAAGTTGGACTATCAAGAA |
| | | TCAAACTGTAATCAGGTCACAGAACAAATGGACTAATAGAGCTATCTAATCATCATACAGA |
| | | TTTATACCCAGTGGAAACAAAACTTTACCCCTTGAGGATTTACTGGAGTTGTGTCAAGTTA |
| | | GAAATCGGTCAACATAAATTAGAAAATGCCTTGGAACGCTGTATAACTGATCACATATAGC |
| | | TGTGCCTAATGCTTCAATCGTCAATGCTGACCACAATCTACCTGACTTGGAAATCCGCTAC |
| | | ACCCATATCCATATACTTAAAGAATCCGTACTTTATATCCTATTCACCGATGTCCGATGTG |
| | | GCGCTATGTGTGTCTAGTAGTATATCAGTTCAAGGCGAGAATGAAGAAGAATACAGGGTCT |
| | | CTTTAGAGCACTGTGTCACTGTTTCTTAGGCCAGTTAATTCTAGAAATCAAATAAATGAAT |
| | | AACTCGCGACGGCTCAAAAGAAATCTATGGTTTACGCATAAGCTGTAGGTACTTCTAAGCT |
| | | TGATTTGCTTCCGGGGGATCCTAATCTAAATGTGAAGGGGCAGATTTAGATCTCTGCTCAT |
| | | TGAGTGGGAGGTTGGACATTGAACATASAACTACCTTCCCTGCGTGCTGTAAGATTATGAG |
| | | AATCTATGCTCGGTCGTTGTCTAAAAATCAGACTACAAGGGTAAGAATAATAACAGACCGA |
| | | AATAGATGTCTCCTTCAAGATAGTCAGTTTGCGCAAGTCTGGCAGGAACGTTAAGTAATCC |
| | | TGAGTTATAATAGCGCCCTTTTAAGCTTTCCTGGCGAAAACCGAACCAAGCCCCCGTAACA |
| | | CAATGTCACTATCCGTACGAAAGTTAGTGTAATAACGACTGTACCTATTATAAGCACATTT |
| | | GGTTGGCTATCTTCTCCCTAGATTCCTGGCGGAAAAGAAGCATGTCTACGTTCGATAGGAC |
| | | TGATTTTTGAGGAAAACTATTATAACGGCTATAACGCGCGATTAATCCCTGTCGGTCGATC |
| | | ATTCACGTGAGTGTAAAATTGTGATTAGTACTTAAACGGGTTCGTGGA |
| 267 | 40.70% | TTGACGATTTATATAGCTACTACTTAGCCTTACTACATATTCCGGCGTGCCGGTAGATATG |
| | | ACTAAGTTAATACTTACAGACATTCAATATTAGGATTTCGGTGACCTCGATCTCTCTTGAT |
| | | TGAATAAAAAATGGATATTAATGCGTCGATAGTTGTGATAAGTTATGTATGATGTCCTGAG |
| | | GGACATATGATAATCTTCTAATAGTTACCTTAAACCGAATTGTGTTTATGATGAAAAATAT |
| | | AGGTGAAGTTAGCACCTATCACCAGACTTTGGGATAGTTAGTCCGTACCAAGCAGCAGTTC |
| | | AACTGACAGGAACGTCAATTCTGTCTCTCATTACTTTGGCCATGGATTGAAAATCGACTTC |
| | | AGTCTGACTCACAACAGTTATAGAAGGATTTTGGCTCACCACTCTTCGAAATAGGTCATTT |
| | | AATGCGTACTGCTTTTTTTGACGGCCCTTTATTCATTCTATTGAGGGAATCCCTAACTTTA |
| | | GCCACACGCAAACTGGTTTATATGGATACTCTCAAGATTGTTTACATATCCAGAAGCTTAT |
| | | ACTTCCTCAATGTGATGCACACAAGGTGGGATCATCTTGTTTCTACAATGCAGAATGAATT |
| | | AAAAATCGCCCTTCCTGGCACATCTTGCTGTACGGCTACAGAGTAAAATTAGCTCGTTATT |
| | | TATGAGTGTTTACACAACCCAAATCTAAGTCGAATGTACTTTAAACTTGGCGTGGATTCAT |
| | | AGACATGCAATCAGTGTTAAATTGTCACTCAAACACGTGCCTGACTTCAGACAAATTCATG |
| | | GATTCAAGCTGCTAATATTCACAATAGACGAGATAGGGGCGTAGCTTTTTCTGTACGATGG |
| | | GGGAATATACGAGCATTTCTATGAACCAAAACAGGCAAAATGAGCAAATACCTTGTGCATC |
| | | ATATAGTTTCCATCAACTGGAGAAAGCCTCTTGATCGGCTACAACTTTTCAAGTCCTTGCG |
| | | GCGTTGGCCCTGAAGTACTATAGCCTTTTGTTCTCACTAATCTAGCCAATCACTTGTTGAC |
| | | TATTCTTGCCTCACCCATAGAGTGGTAATGGAATTCCAAAAACCTATTCCCGAGTTTAACC |
| | | CGTATTGTTTGAGAGGAGTTCCTAGTGTCTTCATTAAATTGCACATGGACTCTACGGAAAT |
| | | TACTTTTTATTAAATCATAGAATCTCTGTCATCAGTCCATGCGTCCTCAGTCAATAACGGT |
| | | CGCCGTGTCTAGGGAAAGGTTCATTCTATGCCTGTAAAGTACATCTAACACAATTTAGTGT |
| | | GGGTCTTCTACTACAGTTCACCCGGGAAACGTTTTATGTACGAGTGTTGGTAAAGCGTCCT |
| | | CATCAAGTCGATCCATTGTAAGGAATCGACTATATACTCCAGCTTAACTAGGACCCCGTTA |
| | | CATCTTAATGGTAGGTCTAAGAGGTGATAAGACTGGAACCTACATCATGAGTTGAGTGAGC |
| | | AATGAGAGCCAGCAAATGGTGGGAAGACTAGACCAACACAGGATCTCATGCTTCCTGTAGC |
| | | AGTGCAACTCAGTTCGCTGCGAAAATAATTAACATATCCCCTATTGGCAAAACCCTGCATA |
| | | CGTATTTAGCAAATATCTGTAGGGGTCGTCCAATAGCAGTGCCGTTTTATAAATTGCCTTG |
| | | ATACATAACACTGAATCAAGTGAAATCGAACGGTGGTAAAATGGCTTGAAAGGGGAAGTTG |
| | | TTTAACATTCGCTAGCGACACATGTTGCATGGTTAGGGTTGCTATTTCGCCTCATTCTCGT |
| | | TACGACATTCTCAACCAGTAGCCCACCAACCCAATTAAGGTCACGCACGAACCTATCATCC |
| | | ACTTAGCTCTTACAACATAAAATAGTCAATACACCTTCCTCAATTAGCCTTAATCAAATAA |
| | | AGCTAGTTATTTTTGTCTCCTGGGGATCAGGGCGCTTACTTCGTACTCGCTTCCCCCGCTA |
| | | GGAAGGCCACTGGTTCCCGAAGAAACGTGAATAATTGCACATGCTTTA |
| 268 | 39.50% | AGTATGGAAGGTGCCTCGGTAATTACGGAAAGAGCTTATCTGCCGGAAACTTTTATTTTGT |
| | | TTCATCAAAAGGTTATACGATAATACCGCATCTACCTTTTCGTATCAAAATTGGTCCACAA |
| | | ATCCAACTTATTGTCATCTTGAATCACACATTCATCTTTCCGTCTAATGAAGGAGCGTCAT |
| | | TACTTGTTGTATGAAACGCAAATTCTCTACACTAGTAAGTGAGACATTAACTACAGCCTAT |
| | | TAAATAATTCAGGTAGACTGATGAGTAATATTTCTTCTATATATATGTGATACTCACTCTC |
| | | TACTGAGTTGACTAGTGGACTCTTTGTTCTTGTACACACACAACAGAGAAATGCCTAGAAC |
| | | AAAGTCAAAGAAAGCGCCTAGATGACTTTGTAAATTGCACCAGATCTGAAGTCGAGTCGTG |
| | | AATAGAACTTTGCATAAGACTCTAGGACTTCCGATGGCGTATTATACTTAGGAAACCAAGC |
| | | CGGTAGTAAGAATCGAGGATAATACTCTGGGAAGTCTTCCGTATTTGCGTGAACAACCAGC |
| | | TTCTGGATCAAGCATTTCTTAACTAGATTAAGCTTCCTCTTTCGTTTTAAAGCGTTTTACT |
| | | TCAGCAATTGTAATCCCTACATTTGTATTAGCCGAATAGAACGATGCTCCTACAACACCAG |
| | | GCCGACCTCATGTTACGATGGCCGAGACCATAACTCTTCGATGAATCATTAGTGGAAGAGT |
| | | TATCTACTGACGGCATGATCCTGGGACATGAAATTGGTAAGCATTTGGACACGTTAATTCG |
| | | CCTTTTACTTCAACGCTCGGACCCGGTATAAGATAAAATTAGACCGTTATCTTCGTAGATC |
| | | GTAATACGTATCATCTCGTATATGCCGCTTGTATTCAACGGTTTCCTTTTTAGACTGGAGC |
| | | GATCTACGCTGGCTTGGTTTAAGGACTATGCTAGGGTTTGTACGTAATCCCTTTAATAATT |
| | | AACGACCGAGCTGACAAACTGAATAAGTACAGCATCAACAGGACGGTTCGATTGACAGCTG |
| | | GAAACCTATTAGGCATCTTGGCCCTTAGCATAAGTCCCAGTATTATTTGTTCCTCCAGTAA |
| | | AAATCTCCCCGGAATTAGAGCAGCGGTGAAATTTATGGACTTGACCTTTTTGGTTTAGTCG |
| | | TAGAGGGACAAATATCATCTCATCTGAACGCTCATCACCAGTTAGTTCATCCAAATTCAAT |
| | | TAGGAGGCGTCATATTGTCGGGCGTCTGTAACGGAGCCAGATCTAGAAGTTCATTGCTATA |
| | | AAGAATTAGTGTGCTTGGCACATCACCTAATCAAATTTTGGGAAGCAGCATAGCTATTCAG |
| | | GTGTTGGTCAACCAGATAAAGTCTATGAAGAAAAAAACCTGTGTTAGTTCTGCGTATTAGT |
| | | ATTGTAGTATAATGTACGACATCCCGAAAGTTAAATTCAGGTCGCAGAGTCCCTAGTCCAC |
| | | CGTTCTAACTCACAAATCGATGTTCGGACATAGCTATTTAACAGTCCATATTTACCTTAAG |
| | | TGTTTCGACTTATGTATGCTAGTTAGGTGTGTGGCTCGCCTTCCCACTGTTAGACCACATC |
| | | TAGACGGACATCGTTAATAATATCTGATATACACAAAAACGTTTACCATAGAAAACACTAT |
| | | ATTCATGGACACTTTATCATATTCCTCGCCCATCCTCACGACCCAGATAATAGGGAGTTGT |
| | | AGTTTTTCTAAACGGTTTTAATATGCAGGTCCATAAAGCATGCAGTACATTACTGTTTAAA |
| | | ACTTTAATTCAGATATATCCTGGAGAAGAAAATCTCGATTGGTTAATCACTTCATTGTTAA |
| | | ATTCGATTTCGCTATACGTTTCTGTACTAGGAAATTTTTCATATTAGGCACGCGGTGTTGG |
| | | TTCCGTAACACTATTAATTTCCTCCCGGTTCGATCATGGCTTGCGGTAAGTCCTCAATTTA |
| | | ACATAATTGAGATACCGAAATCAACCCAGCGTCGCAGTATTTTGAGTT |
| 269 | 39.60% | GGTTAAATGCATGCTCACGCCTCGCCAGGTTGTTAAACCATGTACTTACTCAATTTGACAA |
| | | CTGATGTCCACTCTCCACCTCGCGCGATGCTACTTTCTTAATACTAACGCCACCTTGTCAA |
| | | ACACCTAGATCGTTCTAAGTGTAGCACCAGACAGAGTAGACACCGTAAAAGGTGAAAAGGG |
| | | GATTAATTTCTCCTCCTTTTGCACAAAAAAGTTAAGGGGTAGGCCGGAGGAAGGTTAACGC |
| | | GAAGCACCTGCGTAATCGGTTTCGTGCTATATCGGAGATATACCGTAATGACTCGTCGACG |
| | | AAAGTCGAAGGCTTTAAGCTCCATGCCCCATGTTGGTGCGTTAGGACTTTGGTAAAGTGGT |
| | | AAAATTTAGATCTCTTTGTGTCCTTTATATCAAGTTAGTGTGAATGCTGAGTTTTCTCATT |
| | | TTTTAATGTAAGTGATTAATATGAAGATGTGTAGTCTAATTTGGAGTACCAACTTAAAACG |
| | | GAATAGGATCGGTGTATCAATGCATATGAAACCGGTAAATTTAGTCCTGTTGACCTGAAAC |
| | | TGATGGGAACAAACCCTCAAACGCTATCGCAACACCGTCCTAGGTTCCATGCACTATTAAC |
| | | CTGTTATTGCCCGTGCGGAGATCTGGTTTTTATTGTTTTATACTCTAGATATATTAGCGGT |
| | | TATGTTTTTCTGTTAATTTAAGATGCATAGTCTACTTTGACCTCCGGCAACGTGATTTGTA |
| | | GAAAATATTTCCCACACACACTATATGTGCTACTCAGGTTACCCATAGTTTATGTAATAAG |
| | | TATCACTTTAAACCCTCCACCCGCCCATACAATAGAAGCCCTTCAATTATACGAGGAGGTA |
| | | TTGACCTGACTAGTTTACCAAAGCCAAAGATACCTGGACAAGTTGGACAAATACTAAAGGA |
| | | CTACTGTAGCATAGTGTTTGCGGGCCAGTATACGCTTATTTAAACGATACTACTGATAAGA |
| | | AACACTGGGGTCAACGTGCTTTCATCACCTGTCCATTACTCCAACAGTCCCAATTTTTTAA |
| | | AGAAGGAATTTTCGGGACAGTGAACGCGGAATCGCTAATAATATTCAGATAGATAGCTCGA |
| | | CACAATATAACTAATCAGACAAAAACTATTCAAAACTTTCTCCTAGGTAGTGCGCGGCTCT |
| | | TTTACGTGGGGTTTATTCACCTGCGAATTATCCTGATGCCCAGGAGCTAACTCATTATAAT |
| | | ACCACCAGGTGACAGCCTACAAGTTTCATGGCATGGCTGCAACCTGCACACGAACGCTTAT |
| | | GCAGCATGTGCTCTTGAGTTATACCAGCTACTTGATTCGATATATGGTTTTTGTGAAGAAT |
| | | TTGATACCATTGACACGGGATGTTCCAAATATTTAATAAGTCCATGCATACTAATACCAAC |
| | | GCCAGAGATAGATTGTCAGTAGAACTCTTGAAGTCAATATGGACCGAGTGACTTGGGTGGT |
| | | TTATCCCACTGTTAGAAAGTTATCGTAAAATTAATTCTTGGTCAAATCTAATCCTTATAAA |
| | | CACTCTGTTATTACTCTGCTTCGAATATGTTGTTATTGACCATGCTGATAACTACATCCTT |
| | | TATGTTAATTGAAGGCATTCTCTGAAAGTCAACAATTAACTTCATATCAGACATTTGACCT |
| | | ATTCCTCACTTTTCTATAACATGACAATCACGGTGATTATAAACATGACGCGTATCGGCAG |
| | | CAAACCACTGTACTGATATGTAAGAGCGCCCGTCGCATAGATATTTTAGACTCTGTCCAAA |
| | | TCACTCTACGCCAACTTGAGGTCAGTATGCATACCGTGGTAAGCTGAATAGTTCTTATACA |
| | | CTTTCTAATTAACCCAGATGACGATTTTTTGTTATATGAATGACGATCTTGGCATTATACT |
| | | GCCAAGACTGCAATCTAATCCTAAATTCATTATTTAGTAAGTCTATAGCAGATCTGAATCC |
| | | CATAAATGAATTCTATCGTAGTACCTACACTATGTCACGTAGTACAAG |
| 270 | 39.60% | CTAGGTAAATTCTTAGGTAGCCGAGTTGAACTATTAATAAGTCTCGTCTGTGAGTATGTCT |
| | | TCCGTTAGGTATTTTCATATAGCTTCATGTGCCTGTAAAGACAGAAGTATAATTGGATACA |
| | | TCAGACTTTTTATCCCTTTTACAGTCTAGAAAGACCTACTTGAAACATGTTTCTTAATGGG |
| | | TAACGTAGTGAATTATGCTCGTTTTTCCTTTGGTAGAATGATATTTATCTCCATATGCTCT |
| | | GAGTTGGATAATTTGTAAAGAATTATACACGTTAATTCAACCTCTTTATCAATGAACTACG |
| | | CGGGCTTGATCAGAGTAAACTCACAATAGTATCTTGATCTTCACAATCTGATGGATATTGA |
| | | TGCGAGTTATACGACCTGTGGCATATCAACAATGAAGTGAAGTGTCTGTCCTTATGATTCG |
| | | AAACAAAATAAGTGTCCTTGCTAGCTACACCCACACCGCGGTGTGCATCCCATAAAGGCTC |
| | | AGGTATAGTCTTGTCATAAGCGCTACACTGCCATTCGTTTAGAATCATTGTTTAGCAATCT |
| | | CAAAAGTAATAACATCCGACTTTCGAATAGGTTCAGTTTCCTGATCTACTGGAGCCTATAT |
| | | ATATGCACAGACGAATCTCGTACATGGCATAAGCAAGTCATGAGAAGAGGCTGTACCACGT |
| | | AAATATAAGCCTCTGATTACGCTGAAGCTTAATAATCATCACCCATCTACGAATCCGATTG |
| | | AGGGCATAGGCTTTCATGTCTTTTTCGCTGTAGGTCTATGCGATTGTGAGACTATTGAGTT |
| | | TTCCACAATATGGTGGTAGGTACTGAGTAGGGTACATTTCACTGTCCTATTGCGCTGTCGT |
| | | ATGTCTATCCGCCGTTGCCGTCGTCGATGTTATACCATTTGACTAACAGTGTTATGAGTCA |
| | | CTCCCTTGGATGCGATGTACCTTCTGTTCTGAGGGATGTAAGTTGCAGTTAAGCACTATTA |
| | | GCGAATAACGCTAGGATTCTGGAAGAAGAAAACACAGGGTCGCTTCAGGTCTCGAGAATCT |
| | | TACGGTTAGAAAATTTGGATCTGAATAAAGAGATGTCTAGCCAGTGTGGGGGTTGAATAAG |
| | | CTAAATGTCTGCAATGTGTATGCTTCTGCACAGATATTAACAAATCCGCCATATTTAGGCA |
| | | CATTTGGTAATGGCTGACAATCGGATCTCAAGAATTCTATACTGAGTTATCGGACTACAAC |
| | | TAAAAAGATGCTATATAAAATTGTCATAATTCATGAAAAGCCAGTAGGCCGACCATCATCG |
| | | CTCTAAGTTGAGTTGTTTGACGCGAGGCAACATTACGTGCATGGACGATATACACGTTACT |
| | | AGTTGTATGGTATTTCGGCTAAGTTTCCTAGCTAATTTCATTAAAAGCTGCGCATTGGTGT |
| | | TTTTCAGCCTATATACTGACGTAGTAAACTTACATACTTAATTATACTAGGTAATGATATA |
| | | GAAAATGGCTGTACATCCTTTCTGAAATGCTTCCATGCAATGGTGCTACAAGTCTTAGATT |
| | | TACATTATAATCGGAAAAACATCAACAGTATGATTACCTAGGAGGAGCTAGCATATCCAGA |
| | | AAGTAGAATAGCAGAAGCCACCAACAGACTGGGTGAGAGTGACGTTATGACGGATGGATCA |
| | | TACCCCATCTTAGGAGGGTCAGGTCATTTCTCAATCATATGTTTCCAGATGCGATGCAAAG |
| | | ACAAGGCCCAGAAATTTCAATTGTAGGCCAATCGTCCGGTCGTATTAATCTCAACCAAGTA |
| | | AATAAAAAGCATGTGGGCTGGGCGCAGTCAAAGTCGCTTTTCTTGGTCCTTACTAATCTGA |
| | | AGAATATACAGTAAACAGAGGATAGTGGGGCTAGTTCAGAGTAATAGGCAACAAACCCTTT |
| | | CATGCATTAGTGTAGAATTTGATACTATTGCGTGTATCGCTTTTAACTTTATAAAGAGTCG |
| | | ATACAGCGCAGGCTCATAATGTTTGGAGTCTGTCTAATAAACATCTAA |
| 271 | 40.50% | TTTATCTATTTCATATATTGCTAGATAAAGTTGACTGACTTATTACGATTATTGTCCCAGA |
| | | CAGCCGAGCTGGGCCGTGCGTCAATGCACGGGCTCAGCCTCCTAATGTTAGCATTTGTTAC |
| | | TCTTGGAACATTTGGATATAGTTGATTTTTTGATAGTGCAAAGGTTCTCGGTCATCCGGTA |
| | | TAACGATACTCCCTACCCTAGACATTCAATCGGTGCGATGGTAAGTCCGTTTCGCACTGAA |
| | | AGCCTGTAGAGTCTATTTGATGTTTACTTAATGCGATTTGACTCAAATGTAGGTTAGGAGT |
| | | CCGTTCGCATCCTATGCAGTGATAAACTATCTAGTGTGTTTAAAAAGACGCAACCACTACC |
| | | AATCAGACCAGCAAATTTACATCAATTTATGTCAAAACGCCCTTACTTCGTCTAAATATAG |
| | | ATATATCACCACATCAAGCCTGCACTTCTCACACTATGTTCTATGTCATGTCGTTGTACCG |
| | | AACAATTGATATTTAACCGGAGTTGAAGATCAGCTAAAGAGAGAAGTTATATAACCAACAA |
| | | ATACAGCCCACCCATCAATGATCGTGAAAACAAACTGTACTTAACAGTTCAAGAACAGTCA |
| | | CCATTTCTCGACGTACAAAAGATTCTTCCATTATGGTTCGATACAAATTGTTCAAACGCCT |
| | | GTCTATAGCAGGGCTCCGCCATATTTCGAGCATACTAAATCATTGGGTGGTCAAACAGTCT |
| | | CACAAACAGGTCTGTTGCGATTCATACGAGACGACCATACTTAGGCCTTGAAATGTCGTTG |
| | | CATTTAAGTAACAAATACTATAGACCGCTGGTAGTCGCCATATAACTCTGGCTCCAGATTA |
| | | TACATGACCTGTTTAGAAAGGCAATGGGAAGAGGGCAAAACCCCAAGATTGTTCCTAATAG |
| | | TTGTAGATAAATGGATGATATCTGCATCATCACTGTTTAGAGAATCCCGCTTTCCTTTATT |
| | | CGGTTATACTCACCGTTTCTCGGCGGGTTGAGACATGCATAACTTCTATCTATCGTTGAGA |
| | | ATTATCAACTTCAATTCCCGAGACTGTCATTATCTATAGTTGAGGAACCTTCGTCGCTGCT |
| | | ATTGAATAGTAAGAACCCCTCTAGTCCAGCTGATGCTTGTGGTAACTGCACTAGTAATTCA |
| | | TCTGCCATCCGTGCTTAATTGGGCATGCTTTGTTGCATCCCACTCCCGAACTTGAAGGTTG |
| | | GAACTCTCGTTTTGCCAGCACAGTTAACAGGGAGTAAGACCTATTGGTGTGACATAACAGT |
| | | TAGGTAAATCCATCTAAACACGTGTGTTTACTAATATTCAGTCGGTGGACTAACAGACAGG |
| | | AGCTTACCCATCCGTGGATGTTTTCTTAAGGGTGTCGTTAGAATGAATAGTACATGTATAG |
| | | TACTGTCCGAGGTGTAGATAGAATAAATGTGACCGTGATCTCAGATTTATGGTTCAAACGT |
| | | TCTAATTTTCCGAGGAGTAGTACATGTTGGTACCTTTTCACATTATGGTGCTAATTAGGCA |
| | | TGTATAATATCATATCATAGCTTTGCCCATACTGACTATACTAAAATTGCTATTTTGGAAA |
| | | GTTTATAAGGCCGTTTCTCATTGTATCTAAGACCTAAGCTTCGCGTCAAGAAATACCCTTA |
| | | CAATCGGCCTATTTAAAATTATTCATTTGTCTAGGGCGCGATGATCCTTTCCGAATATTTT |
| | | ATCGATTACTACTTATGGATACCCGTTAGACGCTTATCCTCCTACTACACCGTACTAATTA |
| | | CGTACTTTTTTCGAAGTACGATCTGATTAGTGTCGACCACCTTGCCCTTAAATCTGATCGC |
| | | TCCCACCAGTACGCAGGACACACGTAACGGTTTCGATACCCAGCGAGATCAGCCTTACCAG |
| | | TGCTTGTGTGGTATAACCACACTATTTCAATGCACAATGACAAGAGTACTATGTTAATTCA |
| | | CATGCCTATCTAGTTCAATTACGTTCAGACTCATAAAATGCCATTGCT |
| 272 | 42.00% | TCGAAATTGGATCGACGGAGCTAATACGCAAATTATTTGTTTGTGATTTCTATCGCGCTTC |
| | | AAAACCTACAAAAAATAACAGCCTTTGGTGTAATTCGTCGTGGCCATAAATATGGCTTATT |
| | | CTATATATCCGAGGCCCAGGCCATAACAAATTCTCCAAGATTTACTAAATTAGTACGGCCT |
| | | TCATTCCGACGGGAAGTTTAAACTCAAGCCATGGAGTCCGGTAGTCTTTCAACTTTGTCGT |
| | | ATGACGGTATGCTAGATGCCCCCAATCCGCTATTGAACAATGGCAAACACTACAGCAGTTA |
| | | GCCAGAGAATTACGCTCTTTCACTTTCCTAGAAGTACACAAGTCCTGAACCTACCAACTGA |
| | | CTGTACACACCCTCTATGGTACTTTTGCTGTTTAGTTGCCGAATGATGCATCATGTCTGAT |
| | | TTTTCGGGCTAGCCTTAGCTGAGTGTCAGCTTCACCCTGATAAGACAGGAGTCAGAAACGG |
| | | AATTTCATTAATACCGCCTAAGGCGAAAGAGAGGCTGTCATGTAAGCCGGCAGGTTTCCCC |
| | | CTTACGGGGCCCACACTCTCCCCTCGCTATGAAATGACACTTCACAAACAGTCGCTACTCA |
| | | GGATTTATTCCAAGTTCCAACGATGTTGAGTACATTGAGAATGTATTATATTAAGCTAATA |
| | | GGCAGTTTTCTCCAACTATCGATTATTCGGCTGATATAGCCCCCATCCTGAGACGTTATTA |
| | | CGTCACTGAGGATGATCTATTCACACAACACTTGGGTTACCATAGTTCGGAATGCGATCTA |
| | | ACGTCTCACAATGGTTTTTGGTGGAAGTATAGTCTTATTCCCCGGGCTATCGCAAGCACCC |
| | | AGGAGTAGTTTCGTTGGTGTCATGCTTATCCCTACGACCCACCAGAGTGTCCAATCAATTT |
| | | ACACCTAAACTGGAACCTAATATATTAATCAAACTTTAAATCTCTATATATTCAGACTACT |
| | | TTACTCACTTTGATGTTAGATGCGTAACAAGCATATAAACCCGTTTGTGATCGTACTCAAT |
| | | CGCACCCTTCTCGTTATTGATTGATCCTTGCGCGAGGTAACCTGGGTAATCTCTAAGTTAT |
| | | CGATGCACCGTATCAACATTCATGATCGAAAAAAGTTTAGTGAGAAGGAGTTAATGGATCG |
| | | TTCCGACTAAACTAATGGAATTATGTATGGGATGTATTTCGTTTGAGCGAATTAACTAGGA |
| | | ACTAACTCATACATCTTGCAATAGTGGTAGCGTAAAATGGTTGAACGTAGTTGAAATAGTA |
| | | GGGATACGACATGTCCCCTAAGCCTCACCCTTGGTAGTTCTCGTAAGCGGACAACGCGTTA |
| | | TCATCACGCTTTGGAGTGTACTAGTTTATGTCTACTGCGTTCGCTGACAATAAGAACAGCA |
| | | ATATCCCAATTCTCAGTACTGACGTAGGACCATTAGCGCTATAAAAAAAGTAGCGTGAACT |
| | | GTCATTTATTAAGCATTCCATTTTATCCAGTGTCCGCTAGGCGGCTAAATTATACAAACAG |
| | | AACGGTGTTCTTATACTGTTACTACCTCCACAAGTGGGATTTACGAACGCAGAAAGAGATA |
| | | AGCTCACTCTCGCTATGTGCACCGATGAGTCATACAGAGGTCATCAGTAAAGGAACTCAAT |
| | | CTAGAGTTACAGTCCAGCAATCCAATCCGGATGCCAACAGGCGTAACGATTATATTCAACC |
| | | ACTAAGCCGCATAAAGTATCGATGATTAGCGGGGGAATACCTCCTAAACAGTTTGACCGGA |
| | | ACGTCTACAATACTTTGCCGGTTATCAATGAAATATGCGGGGACGAACCATGCATCGTTAC |
| | | TCAGCCTTTGGTGTACGCCAGTAGGAGTACTACTTGTTCTTCTTACACGACACGTAGCTAC |
| | | TTCTATGTATAGTAATGTAGTTGACTATAGAATGACGAATAGAGAAGGGAACCAGAGCTCA |
| | | CTTATTCCGTGAACTCGATTTATCATGTTGTTAAAAAAGATAAAATGT |
| 273 | 40.00% | GCTACTATTTTAGATATGCATCAAAGAAAAACAAGGACATCTCCTGTATACGTATAGGTAA |
| | | TAAGAAGAGGATCCAACGGAAAAAGCCACCGGTGGAGATAATAACTATTGTTAGCAAGTCC |
| | | AGTTTTCTGTCAGGGGCAACGTTAAGATAGAGGCCAGGGTAATTATTTAACTACTAGCTGC |
| | | ACTTCGACTTCATTTTCTGAGCTCTGTAAATACCAATGGAGCGAGTAGCTACGGTTAAACA |
| | | GATATCGGCTGGATGTCGGTGGTAGGAAAATGTGCCTGTTGCGGCTGATAAGCATTAACTT |
| | | ACCTAAACATAGATTGTTGGTTTTCCTAAGGTTTTATAAGAACGTATATAAAGATTTCTTA |
| | | AATGACAAGCTTAGCCTGCATAGGCTACATGTGAGTGTGGATGGCTTCGACAGTGATCCCG |
| | | CAGTGGACCAGATTCCATTACCTGAATGAAAACGTTCAATTAAACCACTTACCGTATCACT |
| | | CTGTCCTTGTAGCCCTGTAAAATGAGACTTGCGGATACCAAATTAGCCAAATTATTCATCT |
| | | AACTATAATACTTCTTCCATGAAACATTAATACGGCCACCGGGAAGCCACCGATTCTGTCG |
| | | CCTTATATTTTTTGCTCTATGTCTTTCTTTTAGTCCGACAACTAATGTGAACAAATTTCGA |
| | | CCTAACAAAATAGAGACAAATAACCCTATATTAATACAACGCTACGAAGATCTTCAATAGG |
| | | ATTGGTCCGATTATAGACCAATTATACTTTTACATAATATGTACAAAACATCTCGGCATTC |
| | | GATGGCATTGGCGTGGATATTCGATTGTAAAAGCAATGGATTTTTCTTGCGCTGAAAATGA |
| | | TGATCGCCCTCGATCATCTGTATAGCACGGGTCGAAGTTTCAGAAATGATAGTTGCTCAAT |
| | | TTGGTTCACTTCGAATTTACGCTGATGTCCCAAGCGACATGTCCCCGATCAACATGGTTGT |
| | | TGGATATCAAAAAGCTGATAAAAAATGTGAAAGGACACGCCTCCAACGCGTAACTGTTTCA |
| | | CCTACTTCCATTTCGAGGAACTGGGTCGATTTAACGACATCAAAGTTGTTTGCTCAGACAG |
| | | TCTTCCTATGAAAATGAAAAGTGATCTAGGAGTAGAACCCGATGGCTATTAATAAACACAC |
| | | TCTTACTAAATAATTTGGCGAGCATCAGAGCGTAGGTACTCGGAACCTGATTGCCGTTCCG |
| | | CTTTCTATACACTGTGAATAACAAAGTCATTGAGGTGACAACCTTGCCGCGTGCACGGTCT |
| | | AAAGCATGAAATTTTAAAGCAACAATCAAATCTCTAACGGCCTATCTCAAGTTACGCAGCT |
| | | GGCGGTAGGTGGGTTTTCGCACTGACTCTTTAACCAAGCTGCTGCTAAAATACTCTTACCT |
| | | GACTGTTGATATAATGGTCGCGATTACAGATAATCCCGCACATCTGTCAAATAGAAGATCC |
| | | ACTAAAGAGTCCAAATCAGAGAGACCCAATAAAGTAACCAAGGCATTACCGTTTCACGAGG |
| | | TGGACTTTCATGAAAGCATAAGTATGGCGTATAATATAATGTTATTTGGAAAAAAGATCTC |
| | | CACAACCTGTTTTACCGCTGAAAAACCTAAATACCGTACCAGACGAACCACTTGATAGTCG |
| | | AATGCGCCATTGAAGGAAACATTCTCCGTTAATCTGATTTTAAGCTCATCAGGCTTTTATC |
| | | TTTGCGTTATCTACATTTGACGATTACCAAGGATCAATTACGTGATTGGACTATACTTAAT |
| | | ATCAATGTACGAAATCGTCTACGATACTACAAGGTAACCACTGATAATTCCTCATTGCTCT |
| | | ATGTTCACACTGACCTTGCTAATCGACGTGGACTTGCGTCCTTGTCTAGCTTATAATAGTG |
| | | AGATTTAATGACAATGCTGGTATAATACCGTGCAACTACACGCATAGAAATTACTCAGCGC |
| | | TCGAGAAAAGTAGATTACTTCGCTCCTTCGGAGTTTTGCGTATTTTCA |
| 274 | 41.00% | CCTCATTTGCCCTTTTATATTTACCCGAGTTAGTTCACGAATGTGCCATAATTCTGGTCGC |
| | | AGCAAACTGCGGTGTTTAGAAATAATCTTCCGTTATTCGTTTATCAAGACCTCGTTGTTTA |
| | | GTAGTTCTAGCTGAATGCGGTCTATTAAGTTGGAGAAGATCTGGGTTCATTACATTAGAAC |
| | | CCAAACTAATTATTAAGTTCTGCTCATTAGCATTAGGTAGAATCTATTCTTGTCCGGCGCT |
| | | GTTGCTACTGGGTTTAGTCTAAGTAGTACTTTAACTGTTCCTAAGGGATGCTGCAAAATGA |
| | | GATATACTCCTCCGATAATGATCAATTTGGATTTTGGGCAGCGGTAAATGTTTTATAGTGT |
| | | GAATTGTGTTACTAAATTTCATGACGTAAGCTGACCTTCTAACCGTCGTGCTTGGAGGATT |
| | | TACGCGGCGCCAAAAAGAAATATACTAGTCCCAATCGCACTAGGATTTGTTTAAAAAAAGA |
| | | CGGAAAACCTGCAACCAAAGGTGTCTTGTACTGACTCTATCTGCAAAATTTGGATGTTCTA |
| | | GCTCCGTTTATGGTCGCTACATGGAAACGCTATTGGTTAAAGATTCACTATAGGCCAGTTC |
| | | AAGTTTCCCGAAAAATCGTGACGGACGTTATACTCTAACATTGATAAGAACCATGTATCAA |
| | | GCGATCCGCAATATAGGGAAACACGGCGAAGATCAAATTTATAGATGGGAGGAAGCACACA |
| | | CAATATGAGTATTAGTGTGCTGAAATCAGCAGCGTAAAGTGCTTCTGTTCCACCTATACTT |
| | | TTACGAGTCTCGTAATAGCGTATTACCATGTAAGATGCATTAAAGCTATAACTTTATGGCA |
| | | AAAAAGGTAATTTATTCGCTCATTACTATTATTTGTCGTTTTGCATAAATAAAGTGTTGTT |
| | | ACTTCAGGAAGCTTTAATTCTCTGTCTGCCTTAACCCGAATTCTACGCGATCTCCGTATAG |
| | | GAGATGAGAACCGGTGACACGAGACCCGCACTCGCAAGTCGTTTCTTGAGGCTAACGACAA |
| | | AATGAAGCCATCAGCGAAATCTCATCCGTTAGGCTACCCAAAGTTAAGACTTTCCCTGTAT |
| | | CCCGCTAATGCGTCAATTGGTAGAGGTATCGGGATTAGATATTGAAGACCAAGTCAGGTAG |
| | | AGTTGGCGCTAGTTGAACATGGACCTGGCCTTACAAACAAGAAGACCACGAGAGCCCTAGT |
| | | ACAGGAATTTATCGGAAAAAATAAGAAAATTAAAATCCCCGATCTGTGTGGTGCTGAAATA |
| | | AGGCAAGGGCGCTTAGCCTCACAGTCGTTACTAAGTCAAGGTTCTAAAAGCACGTGTTTTA |
| | | GCTTGATGGATCATGACTTCGCTACGGTCACTACTCCACCGTGTTTCTGGAGGTATGCAAG |
| | | GGAAAATCGAGGGATGTGCTCAAATCTGTGGCAACCGGAGCACCATTCTAGGTAACTTCCA |
| | | TTAACTTTTGATTTAGAGTATATGGTTAAGCTATTAAACGTTTCCTAAGGACAAGTGGGAT |
| | | AGTGATATACTTTTTTCGGCGACATCAATCCAGGATTATCCGCTAACAGATCGCCTAGCGC |
| | | TAGCGCATATGATGATATCCTTAGGAAGAGATCCACCCCGGCCAAGAAACTCCACACTCAA |
| | | TAGGCGGTGACCTATTTGTGAGTTATGCAGATGTGTTTCAAGACTCAACGCCGACAAAGTT |
| | | CACCACCAGAGAGTGTAAGGCTTATCAAATTTCTGATTTTATCGACTTATAAATTTGACAC |
| | | GTCTAACAGATTCGGCCTTTGATTGTAAACATCGCCGCTATGATATTTTCGTGATCCTTTG |
| | | GGATACGAGATGCATCAGTACTGGCCCCGAATATTTCCATTTTAATTACTGTGTAATGCTT |
| | | AGGTTCACAATCAACAAGTAGTTCGTGAAAATGTTACTATAATATCCACACAAAGATTTAC |
| | | GCACTCTAATGGTGGACGTTGGACCTCTGTTAACCCGCTTTCGTTATT |
| 275 | 40.00% | ACTAAAGTCCTGGAGAGTATGCTTGGCCTCGTGCGGTAACATTTGAACAGCATGCTAGGTG |
| | | CTAGTAGACCCTTTCTTGACAGCGGAATTTGCTGTTATTCAAACCACCTGTCAGGCCAATT |
| | | CTGGAGCGCAACCCACAGTGATAGAAGATAGTCGTTACAATCAAATCCCACAACTTGAGAC |
| | | TAGCCCTCAGACTGGAACAGTACACAGTTATGCTGTGGAGACAAATAAAATACGTTATGTA |
| | | TTGGTCATTAGATTTGGCTTTCTTATACGTCGTGTAGTAATGCTTGTGATCGGTTGCCGAC |
| | | ATGGTTACGAATAGCTGTTTATTAATTTAAAATTCAATTCTGTCGATTTAGAGGATGGATA |
| | | ATATCCGCTATGTAGACATGAGTGAGTTCCTTATCCTTCAATTCCCTTTTTTCTGTTATTT |
| | | TGGATCTACGAATGAGGTATTAAGTTCGTAGCACTCGTCCGTTTCGTGGAATGACTTATTC |
| | | GAGATGGCTTGATAAGGAATTGTACCTCAAAGGTTTCATTGTTAAGAAGATGAATTTTCAC |
| | | GCCCATGGCATAAGCATATGATTAGGTCCACTAGGTCATAGACACATGATAACTCGTCGCT |
| | | CAAAATAATCGAAAGAACGTCTATCGGCCAAATTATTACTTTGATCCCAAAGGAGAAATCA |
| | | TATTGGGGCGCGGGACTTCATGTGTATTACCATCCAGCAAGCATTTGATAAAAGTAACTCC |
| | | TATATTATTATGAATAGCGGTAAGTTTCTTTGACCAACCTGACAATAACACCAAGTGACTC |
| | | ACTGAGCCCGTTATCTACTAGGTATTCGCGAATACCGTAAAAGCTTGATGCAGGTGACAAT |
| | | GAGAATTATCATTAGCGTACTGTATGCTCAACCTAGCCTCCTTGCAAGATTTCGTTCTATC |
| | | TATTTTGTATTCATTTCTTTCCGCGACATGCATTCTTTTGCTAGATCCTGGGTCCTGCAAT |
| | | CATTTATAAGCACGCAACTTAGCTTAAAAGTGTGGAGACGAGACGTACAATCACTACTTCC |
| | | CATCACTTCTTCTCTTATAAGCGTACCGAAAGACCTCGTATTTTATTAAACAATAACGTGC |
| | | AGTTGGCCTAACATAATTCGATGTCTTTCAGTGTTCTAGGAAAGGTGCGGTGTGTCTAGCA |
| | | AGCATGTCAGCCCTACAGATTCTTAACATACCTATGTGTCTAAATCGAGTATACTATAATG |
| | | ATGTACCATAAGCCCTTGCCAAAGGATCATATTCGGACTAGTTATTGCCTTCTGGATGGGG |
| | | TACTTAGACTAACATTTTAAACCTCTTGCGATACGACCTGGTGCTAATACACTATTCCTTC |
| | | TTTTCTCACGCGAACTTTCAGTATCGTACAAAAGTATGGGATTTAAACCTTTTGAAGTTTG |
| | | GTCGTGATTATTTGTTTTTAGGGCCTCCTCGACGCCTCAAATAGGGATTTCTTCAGCACTA |
| | | CATATTTTGAGCCGTATGCGAACCCTTCTTAGGACCGCGGTAGTTTGTTCACGAGCACGTT |
| | | GGCCACACCCCAATTATCCAGAAAGCCGGACTTAAGACATATTGAGTTTGTTAGTGCATAA |
| | | ATAGGGTCGCATATTGATCTGCGACTCGAGTAAATGTCGTACTGGTGATATATTCTCCCGT |
| | | TTTCGAAGGCCCCAATCAATTACTAATTACCCTATTTACGAATGTCGAGAGATGTTCAAAC |
| | | GAAACATGAGGGCGCATCCCAACGCCCATTTTGAAACTTGATTGTTGTATAATTCTTAATT |
| | | TTTGTAGATTCAGCGTTCTTGACACATTTTAAAGACGTCAGTTCACCGTACCTACCCCTTC |
| | | GGTTAGGCGAAAAAGATTAGGTTAACGATTTCTATCGTTCGTTGGTTGTTATTTCTGCAGT |
| | | ACATTAATTTTATAACTTGATATATCAAATCTGTTTTTGATTAATGTTTGAAAGCTAATCG |
| | | TAACACCAAGGAATGCTAAATAATCATACGTGGCGGACCAGCTACTATA |
| 276 | 40.10% | TACATCCCCATCAGTCAAGACGATTCGTTAACAAATATCGCTGACTGGGAGAATCCCAGCA |
| | | TGTCTTGGCTGGCTAAATAGAAGCTACTATGTTACGCACTTCCATTTTGAATTACAGGCGA |
| | | CAACATTACCAGACTTAGTTAATTATTAAACAAGATCACTTTGCGACAGTCCTCTGAGGAT |
| | | CAGTTAGAGTGCAATCACTTAAGTAATACAAAAATACAGAAGGATTCTCTGGCGAACAGGT |
| | | TTATTAGCGCATGGCCAAATTTCTAATCAACCCCTTTAGTTAGTAGCCATTTCTAGCCAAT |
| | | ATCAAATGTACTCCAAGCCGGCGTATAGTTGTCAGTGTGTGATTTAACGAATAGGATCCCC |
| | | CCCCATAACAAATACTAATAAGAGTGGAGCAATTATAGTTTAGATCGTAAAGGTTTAAATA |
| | | AATAAACGTCAAGCACAATTATGGACTCGTATGGGGACAAATTGAGCCTACTAGCAGTTCT |
| | | AGCGAAATAAGTTGACCTAACCAGTCCATGGACTGCCGGTTCGTTGAAGTCGGTCCAACGG |
| | | ATTGCAGATCATTGCTAGGCAGTTGGTAGATAAATTTCTAGTACTTATAGTCACGTAATTG |
| | | TCAAAAGTCCTACGAGCGTGGTCACCGTATTACTACGACCTCCATAGTTTTCTACCGTGCA |
| | | TTCTGAAAGAAATATGGCTGGAGTGTCCTAGCTCATGATAGAAAACGCCTACACTTAGCCA |
| | | ATCAGACATTAATGCGGTAACGGATCAAGCATTACAGGGCGGATTGGTCGCATATCATTGC |
| | | ACGGAAAGCGTTGCCTTAAGTTCGGTACATTCCACTTTCAACTTCATATTGACTCAAATAG |
| | | TGGGACAGTGATTTACGCGGAGTTTTAATCTAAAAATTCTTGAGTTTATGATAGAACAGAT |
| | | CTAAATTACGGTTTTTATATGTAGTGGTATTAATAATGTTCATAACCCTAGATATTTCCGA |
| | | GATTAGCACTCGTTCGGCGCATTGCCGGTATAGAACAATATGTGAAGAAATTTGCACCTAA |
| | | GAAGTTGATATTCTCCTCTACATGCGTATAATATATAGTACCATAAGTGGATCATTATTAA |
| | | AATAAATCTGAGTGGGTGGACTTATCTTCTGTCACCCTAACTGGATCAGCAGTGGGCTAGT |
| | | AGCCATTAAGGAACAACCACTTGGCCCGAAACTATTTGAAAAGTGATAAATACATACACGA |
| | | TTTACTACATAACCACTCCTCTTGTTGATAGGCATGCCCAAGGATTCGTATGGGCGATTTT |
| | | CCATAAACCTACAGGGTGATTCGCGCATATAAATAACACCAAAGCAGTCAGGCTTTTTGTA |
| | | TGAAGTGTAGCTTCCCTAACAGTATGATAGTTGTGTAGAGTCGCTTCTGAACTGGCTGACC |
| | | CTAGTTATAATTAGTTCGGCGGAGGATGGGCCGCGAGACAAAGTATACTCGAACCTTAGGG |
| | | CCGCATTCCAAAGGTTATTTAGATAAAAGTACGCAAACCCGCACATGAGTTGAAATAATGA |
| | | AGTACAATGTTATTTATTGTGCGTGGTAATAGTCTCGTGACTGAAAATTTTTACCTTTAGG |
| | | GTTCTCTATCCGGAGGAGCGTCATGAGCTCAAATACAAAATCGGAGCATTGACTCAATTAC |
| | | TACTTTATGACAAATTCTACGTCTAAGCGATTTTTCTAAATCGCCGTGATCAACAAACTAG |
| | | ATCTACACCAGTGATGCATGCTCACGGCGAATGTCCTGAAGTCAGATCTAATTCTTAAGGG |
| | | TTGGATTAGCTGGCTATAGCAAGCCATATTAATATGATTAGTCGTGTATGGTTTACGCTAC |
| | | CTCTCCATAGATATTTCTAACTTACATTTGTAAATGTTTCCAAGCATACCGTCAGTATAAA |
| | | TACCCAATGATGTGGCTCTCCTTCAAGTGTTTAGATAATAGCTATTTCCATAAGGTGCCTC |
| | | CCCTATCCGCTCATCCTCGGGTTTCATATGTTGTAAGTGGCACTTAGA |
| 277 | 39.50% | TTGTTTCTTGGAGGGTTACTTAGGATTATTCAATGTCAAGCTGGTACCAAATAATATGTTA |
| | | ACATCGACAACGTTGCTGATTCTTTAACTGTACGATTTACTCAATCCTTACAACAGTCTTT |
| | | CCCCCCGATGCTTCCGATAATCCGGATGGAATGTAAAAGCTTTAATTTAGCCATAATGGAG |
| | | CTACTCTGCAACAGTAAGGCAAAATTTTCTTAAATGGAGGCCAGGCAAGATTTGTCCCCGC |
| | | CAGAATAGCCTACTCCACAATATTCTCTTTAAATATTCGCCATGCTATCTCACGCATCCAT |
| | | GAACAGGTTATGAAAGCGTAGAGTCAAACGTACACTTTAGGTTAGGTGCCTTGTGGGGATT |
| | | TCACGCCACAAAGTAGAGTAGAAGCAGTGTATCAAACTATGTGTAAAAGTAATTTCATATA |
| | | GTAATAGCCACCAAGAATGCGAACATAGGTGTCGGCCTGAAGATCTAAAATTATACTTATT |
| | | AACAATCATGTGAGTAGGTTGGATTTTAACACGTTCATAAGTATCGATCGCTTCGCTTAAA |
| | | TAGAATAAAGTACACATCATGTGACGACGCGCTTCGATTATTGTGCTGCGTTAAGAGTAGT |
| | | AGGATAATTTTTGATAGACCTGTCTATAACACGGTATTTAATCCGAAGTTCACTATACAAT |
| | | CATAATAGGATATCGTGTTCTGTCTCGATGATCTATTCGTCGCTTCGGGTGCAATATAGGA |
| | | TTCCTATATGAAACTCACTTCCCTGAGCATTGGGATTTCTTGATAGCTAGATCGCGTTAGA |
| | | GTCGGGCGGTGTATAGTCTCGGATACAAGAACATAAGAGTAATTATGTGGAACCTTTTCAT |
| | | GTGATTGTGCTAACTGTGTGATATTCGCAATAATTCCTACATCTTAGTTTTTAGACTGGAC |
| | | TTTTTTTTCCCAAGCTCTAAGCATACATTATTCGCTGCGTATGTCACTGACCTAGAGGAAT |
| | | AAGTGTTCTGCTGTCAAAACTAACTCTCTCTAGCAGCCTTTTTGACCATATTATCAATTAG |
| | | GCGCCATCCCATAATAACTTCAAAATTTGCAACCATCGGAATTAGAAATCCCGACGTAATC |
| | | AAGACGAATCTTCGCCGATTATCGAGCTTACATAATCGAAGGTGCATTTCTGAACCTTGGC |
| | | TACGCTAACCCTCTAGTCGGGGCAAGATGACTTGGTTATCTGGTTAACTAGGAACTCCTAG |
| | | CCTCATATTGTATCAATCTGATCTAATACAGCGTCTACCAATTATTTGATTAGGTTTGCTT |
| | | GCCCTCATAGCATCGCAGCGAGTATCTCACAATGTGTATGGGTATTCTTCTAGTTACGAGT |
| | | TTAGACGGAGAATAAGCCGCTTGTGGTTAACCTCTGTAAATACCTCTAGTTGAATAAGTGT |
| | | GCAACCCAATTCACATTCGTCATGTTAACAAATCGGCAATCTTTCCACTAATGAGAAAAAA |
| | | CAAATCATTAATATATGTGAAAGTAATTATTGTGTCCTCATAACGGTAAAGACTTACGAGT |
| | | AGGTAACAATCTCAACTTCACCAATTACCACCTAGATTCCAGCACCGCGAACGTAATCAGT |
| | | GTTCCGTGCGTCTTACACAAGAGAACTCCTTAAGCGGCTAGCGTATACTTTTAAGAGCAGT |
| | | GGGTATGTGGCCCGGGGCATCTATTGTTTACCGTAATATAAGCGCACTAGTCTATTTTTAC |
| | | ACTAAATATCATTCCATATCCGGTTCTTTCAGTAACAAAAGTAAACACAGTGTTTTGGAAG |
| | | CAGTGTATCAAGAATTGTGAACTTCTTTCACCGGCGCAGGGATCCACTGTCTAGAGAGAAT |
| | | CTTAATTCTATCAACCGACCCTCCATGTCTTATAGATTGTGTCAACGGAGCACCTAACCGT |
| | | ATCCTTAAAAATTTAGAGGAAATAGAACTCTCATTCTTCAGCCTGTTAAGCCAATTAAATC |
| | | GAAACCGTTGCTATTAGGTGTAACGGTAGATGTGATAAAAGGGTCACA |
| 278 | 40.60% | AGGACGAGCTCTAGGGGTGCCCCTGCTGTTGTTGGTTATTTAAAAGCCGCGATGAAGAGAA |
| | | CGCTAGGGGGAAAAAACGATTTGCCTAGAATAGTGGATCGGCGTTTTGATGTAAGTGTAAT |
| | | TGGGTAGAAGGACTTGTTTTACATTTGCGAAATCTTGCTCGGGGACGTTATAATATGGCCT |
| | | TGAAATGGATGATGACAATATAGTTTTAATGTTATTATAATTAGAGTATCGTATTATTAAA |
| | | AAGGATGTCCACTGTGGATCCAAGTTAAGCATTAGGCGCGTTGAAGAGATTGTACCGCCCG |
| | | AACCAATGCAATTGACATGCCTAACTAGCAAGACAAACGTGTTAAGACTAAAGTCCCTCCT |
| | | ATCAACGTACACCTGATACGCTTGACTAGGTAGAATACTAAAATACTCTCGTAATGAATAC |
| | | CTATTATCTAAGTGACTGCTGCGTTCTTTTAGGTGGTGAACTGGCTCCGGAAAGTGTGCTA |
| | | ATAGTCTATATGTCCGCGCCTGCCACGTAACCACGAGGCGGATCAGCTAGAAACATAAAGC |
| | | CGTTTGAGCAATAAGTGACTATACTTAACGGTCTGTAAATTCGCGCTTCAATACCTCTTAC |
| | | TCTCTGCGTTCTATCCCGTCTTTTTATAAATTCAACTATACGCTCCATTGCTTATCGCCAT |
| | | ATGAGTCCTTATCTACTTAAACTGGCTACCAATTCCTTGCTCTAAGCTAATGAAAGTCCAT |
| | | TCGCAGGATTACAACATCAATGCTAACTTTCTCTTGCATACAGTATATCGTCTAATAAATG |
| | | TATAGGCTCCCGGAGGTCGGAACAGCAGTACTCCCGGCCACGTATCCCGAATACAAACCTT |
| | | ATTAGTAAAGGAAACACTAGTGAGAGCGTACGGGGATTACTCGAAATATCGCAGGAAGGTG |
| | | GTTAATATGCCAAGGAAATACGAATAATTCTCTCCGCATTCCGAAACTGTTAGCACATAGA |
| | | CAAGACAAAGAGTTTACTGACACATCTTTTGACAACCCGCACTCTACAACGACCTACTCTT |
| | | TATACAAGTACGGATTATTGTAACGCTCCAGCCTAGAGAGAGTAACCCGGAGTTATATGGA |
| | | GTCGCTTGAGGAGAAATATTAAAGCTGAATTCTGTTACGACTAGTAACATTACCAGCCGAG |
| | | GTCTGAATAACGTGCCTATGGCGATCAGGACAATACGAGAGAATTTCTTCTACCACACTAT |
| | | GTGCAGCAGCTCACTCAAGAGTCCTATGTAGACTGTTTAACCAGTAAGGATTGTTGTGCGG |
| | | AAGTGTAATATGGTCGAGAAATACCGCTAATATGGATAAGTTAATTGAACTTCGGACGTCA |
| | | CATTCTCCTATAATGAGGATCTATTCAAATCGTTTTGAAGTAACCTCCTCATTTGAGTAAA |
| | | CTAGGCTTGCCTGGAGATGGGGCCCCCAACTGTAATGTGTTATGTTTAGTTTGAACTCAGT |
| | | TGGCTCAAAGTATCCCGCAGTACTAATATTAAATCTTGTTATTGTACAGCTGGCGAAGAAA |
| | | GTTAAGAAATGTGACTCCTATACTATTACTGGATTTACAAAGTAAGCGTCTTTGACATTAA |
| | | TTATGGTATTGACAAATCAAATGAGAGACAGTAAGATGATGACATTCGCTCATATTGTATG |
| | | GCTCGTTGACTGATGCAAATAGTACCAAACCCTTTTTTTAGAATTCCAGATGAGGAATTAG |
| | | ATTTTTCAGTCAATAGTTACTTGTTATGCCACGTAGGCTTATGTCCCCTAAATCGCATATA |
| | | ATAAGATAGAGTGCGAATGCGTGCACGTGTACACTAATCAGGGCAAACTAAACATTTAACC |
| | | TTTGGAGAAATTCCGTGGCGCTGAACTTAGTGATGATATATGATTAAGGGATCCGTTTTGT |
| | | TTTCGATAATCTAAGAACTGACGAAGGCACTAATATCGGAGTTACACAGGAAATAGAATGT |
| | | CGCAAGATGTGCCTTAGGAGTCAGAAATCAACGAGTGTTGATCCCACA |
| 279 | 39.00% | ACAACGACTTTCGAAGGTGGCTGAAGAAAACCACATGATAAAATCGCGAGTATGGTAAAAT |
| | | TAGCTACCTGAGTATATTTAATCGAGGTTATATCTTTTGTGAGTCGGACACAAATTCTATA |
| | | TTTGACGGAGCATAGGGCAGACGGACATATAAAATTATAAACAGTCTGTACGGCGGGGCCT |
| | | CCAATTGGATTCCCGCGATCATATCAGTCAGTTGGGAACCATAAATTGCGAAACTCAGTAC |
| | | TATGCTTCAATGCCCCTTTCTAACACGTTTATCGCTTCAACCTAACGGTATTTGCACTCCG |
| | | ACTATCGTCTTATGCCTCACAATCAGATGTAATAATGCGGGATTTATAAAGATTTTGAACC |
| | | ATTGGACAACTGACGGCTTCTCATCTCACCTTGACGAGAGTATTTCCTATTAACCTGAATT |
| | | TCGCTAAATACTTATCTTTATCGCCAATAATTCCTTTATGATACACAGGGCTTCTCCAATT |
| | | CATCCACGCAGAAACTGCCCAAATGAGGAGAATAAAAAACTTTATAATTAAATGAATTTTA |
| | | TAGCCTATGCGTATCCCCCTACTTCAAATCTGTGCAGTGATGATAAACTATTGTAATGAAG |
| | | ATCATTTAATTCGCGAGATTAAACAGATTCATGTTCTAATGCGATTATTCTGGTGTGATAT |
| | | CGTGCATGGATAATAGAAAGCTGATCCATTTAGAAACCAAGCTTATGCCTATCCGCACCTT |
| | | TAACACACGCATAGATTAGCGCTCTGCGCGAATCCTGCGCGTTGCAACTGTACTGATACAA |
| | | TGCGCACCAAAACAACTTATACTCTAGCAATGTACACACATATTGCGAGCCAATCTGTTCA |
| | | GTTTCCCTTTGATATTTCAGGATAATGAGATGGACGCCAAATAGATTACTCTTATACTGAG |
| | | GAAAATATGAAGTTCAGGTTCAGCGTTACACGCAAATCAGCGATTAGGTCTGCCTAATATG |
| | | ATTTACGTAAATAAATCTACCAACTAGAAATCCGGATATTTTACAATAATCATGGCAACGG |
| | | GTATGACCACTGGGTTCGATCCATATACCTGATGGGCTCGGCAAAAGTCTGTAAGAATTCT |
| | | CTACATCCCGATCGATGCTTCTTTATTTATTTTACTTCATAAACTCGTATTTAAGCTATGC |
| | | ATTGCCAACAGGGCTTAAATAAGAAAAAGTGTTGCACACAGAAGTTGCTATGCCGCAATGG |
| | | AAAGAGTACTTTCATGAAAATACGTAGATATTTAGGAGCTTTCATTTAGTAGGTCATCTGG |
| | | TTGACCATATACTAATCGGATACTTGCGAATTATTGTCCTTTCAGCAGTGAATCCTGAGAC |
| | | TGATAAGCCAGCAGGCGGGAATCGTATTAGTAAAATTTAAGGACATCTGAGTACGGGCGAA |
| | | ATCTACAACACGACGAAATCATCAATCTATTATGACATAAGTATTGGACAGTACGTCTGAC |
| | | TGGGAAACATAGCTTTATGTTGGATATGTACATTAGTGCAAATCTGTGTTACGTGTTAAAT |
| | | CATCGCGTTCTAGAACTCTTAATCACATAGCGAGCTACCTTGGCGAACACTCGTTACTGTT |
| | | CTCGTTTTGCTATCATGTCCTAAAAGCGGCAAAAGTTATTACTGCAGGACCGAAAAATATG |
| | | AAAAACTTATTTTTTCATGGGACTACACAAATCGAGTTGAGCCTTTAAGCGGTTCTATGTT |
| | | ACTTGAGTATCTTGAACTTGGAGGGGGGTTATAATGATAATAGCAATACATAGGTTATGAT |
| | | AAACTGTCCTGTTTTAGATACACGGGAGCCTTAGTAGGCTTATTTTAATAGTGTAGTTGTT |
| | | GATATGAATAATATAGAAAGGCCATGGAGGAGAAGTGCTATGTTAAGAGGGCAGTCGCGGT |
| | | CACGTGTGCCATTGACGCTCACTTATATGCTGCGTTTTCGCAGTGTCTCAAAGATTAAATT |
| | | AGCCATATGGTGTCTATTGTTTTCGTAAACGCCTAGCATGCGTTCGTC |
| 280 | 38.90% | CTTGTGCGTCGAAATCGAAACTCAAATAGTATGTACGCTGAAAATAATAAAGCCTAGCTAA |
| | | CAATCCATCCGCGTTTAGATCGTAATTCACATTTTACCGATAAAAAGTTAAGTACAACATT |
| | | GGAATTGTTATTACTTAGCCAGCCAATAACGCGTCCTAATTACCAAAAAAAACAGACTCTG |
| | | AATCATGGTAGATTAATTGGGTATCGATAACATTATCCAAATTCAGGGGGCCATTCGCTTA |
| | | AGAAAAGAGATGTTAACGTACTCCAGCGATCTGCGGTGTTCTGACTGTAAAAATACGCATA |
| | | CATTTCACCCATAGCAGAAGACGTAGGACGTCTTTTCTACCAGGTGTCTGTATTACATACC |
| | | CCATGCATATCTAAAAGGATTCTGCACGTATTTTGATTTTTACCAGTTGAGATAGTGTCAA |
| | | ATTCTGACTTTCAAATGACAATCGCAAAAATGTATGCGAAGGCTGATGATCTTGTAATCAA |
| | | TACTGGTGCTAGTCACATACTGTTGTAGATACGCCAGATTTACACTATACACAGTGAACAA |
| | | GGTCATGTCAATAACAACTATTTTTGTTTATAATCACTAACCCTGCATATGAGGGTCTTGA |
| | | TCCAAGTTCGAATGGTTGAGAATTCCGAGTTTATTGGTTAGGGAAGATGTATCAAATATAA |
| | | TCCTTGCTTACTTCCCAACAGTCACAAGAAGCAGAGTTAACGACTGATTACGGCTGGACCA |
| | | ATAAATATTGAAACATCGCAATAAAACTTGAAGAAATTTGACTACAAAGTTTAAGTGTATA |
| | | CAGTAGATCGGTTAGGGTATACTCAATTAGGGCGGAACCCGCATTCCTGTCGATAAGCTAG |
| | | TAGTAGGTGGTTTTCAGGTTGGTATCAACCATCAATATTCGACATACATTAATCCAGTGAA |
| | | TAGGGGCGTCCGGATTTTGTAAAGCATTAACCTTCTGTATAAATACTGCCAATCATATGGC |
| | | TTGAGTAACCGTTTTTGTCAGTGGAATCGTCCCCTCGCTAGAAGCATCTGTACGATATCTA |
| | | ATGGCTGTAGTTGCCTTAAATCGGAAAGGTAAGTCGGAACCTGGGCTCTCATTCGAATAAG |
| | | ACCAATCCTAAACGGCGAATTCCTTTATCTTGTTAACTGCTGTGTCAAGTCCTCTTATCGA |
| | | AAATTCTTACATGTTTACTCTTGCGATTAACTATGGTGAACTAATCCCAACAATGACTGTT |
| | | CGTAATAGATGTGTTTGTAAAATTAGTATTTTGGTGACATCTCTAGTCATTTCATGCCTTC |
| | | ATAGATCATCGGTATTTCGCAATAATCTGCTCATACTATGTACAGAAATACCACTACCTTC |
| | | TGACACCCTTGCTAGCACTCTGGAACTAAATAACTCATAGACGAAAATACAATGCAAAGCT |
| | | CATCTTCTTTTGAATATTGAGCGAAGTAGATTGTTGACGTTAAGAAATGAGTAGTTTCATT |
| | | CGAGAACATCCGTAATCAACTACAATTATAATCTCACAAGATCGGTCTATTAAATCCCTCA |
| | | TACTCCTAGGACTAGAACGAACGATCGAATTTGTGCTTTGGGCTTAGGTAAAGACGTATAA |
| | | TCCTACCTAGAAGTTATCCATTTATCCACTTGATAACATATGTCTATTCCCCAATCATAAT |
| | | AAGACGTAGAAGAAAACGACTCTCACAACGACAGTATGCCCTAATATGCGATGGCGACTGA |
| | | AAATCTTACGGCGCCCGCCTCAATCACGTTCACGTGACCCAGCACATTAGATCCAGGACTG |
| | | ACTCAAGATCATTACTCGGCGATCAACGCACTATCCTCAATTGGCTATGTGCGAACTCCTC |
| | | GTATAGGATAAGGATATTCCGGTCTCCGTATACGCTAGGCTCAGTAACGCGTCTTACTCTG |
| | | GGTCAAGGGTTTAAAGATCATAGCGGTATCATACAAAAAATCATATGGCCTACTTTGTCGT |
| | | TTTAAGCGAAGATCAACGACGTAATAGCTAACTTAATGAGCAAGATTT |
| 281 | 40.20% | TCGATAGGACAGATAAGTGACCGCTTGTTGAGTCTTATATGTATTGGACTTAACATCGAGC |
| | | AACAGTCTGTAACATATGTCACTACGTGATTGAAGGCCGTCGTCAGTAATTAAGGATAAGG |
| | | CGGTAAGACATAAGATACCGTACAAGGATATTTATCGTTATCTCAAGGTCAAATCTAACTA |
| | | TAGGTAACAATTACCTTCTACTAGTAGGGGAATTCCGTTGGATAGCTAGTAAAAGATTGCT |
| | | TCAACTAATCCAACAAAGTATTACATCAAAACAGATTGGTTATCAAGATTGGAGCTTCAGA |
| | | ACTAGAGTGGTGAGCAAAGCACTCTCATGCCTTTTGTAAGAACCGGGAATGAACCGCAAGA |
| | | ATCACTTGACAAAGGTATTGGGTGGTTATGTTGCCGGGAAGCTACGATTATATCCAATAGG |
| | | CTACGGTCGTTGTACAACCGGTTGTCTATCTGGTACTTGGTTGATGACCTAGGTGCGAGCC |
| | | ATTCTGCCAAATTTATATGGAGATTAAGAGTGGTCTTTGCCTGATGAAAGGGCCAACTGCC |
| | | GAAGTACTTTGGAGCAGTGTTGACTGCAGCTCCAAACATCTTGTATTTTAATATTTCGGAA |
| | | TAGACATCTATCGTTAGTGAGGAAAGAATTTGATCCCGCGCTATTTTCCCGACATTCTCAA |
| | | CACTTGGATTACTTAACTCATAGAATTTTCTACCTATTATATTATAACAAAAAGGTCAGTA |
| | | TTGGTCCTGACGTATCTGATTCACGTATTACGGGGCGGGGTGGAAAAACTTGGTTTCCTAG |
| | | AGCCTTAGACGAGCGTTAATATACAACAAACTAGTTTCACATAATATTACGTATGGAGTAG |
| | | ACTCAAACAATGGATCGCGGCGACGTGGATGGTATTATCGCATGATGCAATTCTAACGATG |
| | | AATTTGTGTCCGCGCTGTTGTCGTTTTAACAACGATTTTGAGGTTATGATAGTTATAATCA |
| | | TTAGAACATGTCCGAAATTCAAGTGGTTCACCTTAGCTTTGTCAATTTTGTCACACTTCAG |
| | | GGAGGGTCCAGGAGGAACTGCAATCGTCAGTCTGAATCGTTCGAGCAGTAGAAATGACCTA |
| | | ATTTGCTCGTGACGTACTGACGATACCAAATCAATGATTGAGTTCGAGGATCTGATGTTTG |
| | | GAGCTTGCGTTGGACGATCTGATACTCAAAAGTCGACACTCAACATTTTTTGCCACGACAG |
| | | ATATTCTCCAGACTTAAGAAATCCTTGCTGAATATCAAACATGCAGCTTAGATTAGTTATT |
| | | ATGTAAATTGTGAGATACTATGCTAACTCGATAGTGAGGTGTTGGTCTGACACCGTGAATT |
| | | AATAGGTCGTCCTTAACAAGTACCACTTAGATTCCTCGCTTTTGAGTCTTTGACGCCTTTG |
| | | GCCGGATGCATGTATAAATCCTTTTCAAAAGGCTGTTCATTCCCATCCAAGTTCTGTAATA |
| | | GGTCTATCTTTACTTCTGGTAACAAGAGGGAGTTGGGTTACGACGAGTAATTGTTGTAGCA |
| | | AGGATAAACTGCTATTTTTGATTAACAGCCTCACATATAATACGGGCAGCCAAGTCAGCCT |
| | | GCCGGCAAATTTAGCAGTGTTTCTGCTCGCCAATGTCTCGAGACTCCTAGCTCTCTCGTCC |
| | | ATTGCTGACTAGAACTAGCCAATTCGGCGAGCATTAGAGTGCTAAAAAAATCGGTACAGGA |
| | | GCCTAAGGGTATCCGGGCAGAAGCAAGTGCTGCCAAAGACAGTTAGTTTATGAGCTTACGT |
| | | CCAATGATAGAATTTGCAAACGGTATGGTTACCTTCTTTTCTGTATCTTCTCAATGTAATA |
| | | TGTTAATGAACACATTGTTAATGTGGTTTCATATAGTAAAGTAGAAAACTAGCCGACAACC |
| | | AAAGTAAGAGGAGCAGTTTTAGAATCAAATACACCAACTTAAAAATTTGCATCTATGTTTT |
| | | TGAGAATTGACATACGACATAATAAAAGTAGGATAGTTGTAGATCGTC |
| 282 | 39.90% | ACAACAATCCAGAATTAAAGAGTCAATGATTAAAGTCTCTATAATTCTTGGTGGTTAAGGT |
| | | GCAACTTTTGTCAAGCCAATGCTTCTCTAGCTTACGAAAGGAACTAGTATTACAATTTGTT |
| | | ACCGCATATACTAATGATCAAACATTGTACAGGTACGGTTAATAGGCGCACTAGTAACACC |
| | | GTCAATTATTATCCTCGTCCGACCTGAGAAAGGATGATAGATCGTGCATAGAGGGACTTGT |
| | | GGAACGAAGAACATTTCCTACGCAGCTACAAAAGATATATTGCACCAGGGACGTCACACTA |
| | | AAGATGTATACTACAGCATTGTTTCTCATAACCTCTAGGTAGGTCTGTAGATTCAGCGTAT |
| | | ATCGACTACCTACATCTCGTCTGATATTCATCTATCGCCTTAAAATTGTGTAAAATAATCT |
| | | GAGGTCATCAATGGTTTTGTTTTTACATTATGTAAGGTCCGTAATGGTAACTTGTGAACCG |
| | | ACATAGTTCCCCGTCGCTTAGGTGTGCAGATAATTAGATCCAATGGATCAATTCTCGGAGA |
| | | TAGTCTTCTACGGCATTCTATCTGTACACGTATTGGTACGGGGGTCGTAGGCAGGGAGACA |
| | | TCTACAAAAGTTAGCGGTTGCTGAATTATTAATATACAGCTTTACGCTTATACGGTTGACT |
| | | ACAAAAAAATTACAAGATTCTTCATGAGATTGTACCTGTCAACTTAATTCGTATCAAAAAT |
| | | TCTAAAGTGCGCATCTAACTTCATACAACGGAGAAAAGTAGATATAAGTAGGGTGTGAACG |
| | | CAGATAACGTTCAAAATGATTTAAACTATGATTGAGATGTCCAAGTTAAGGACGGTAGGGT |
| | | TGCTACCGTGGACTATAAACCCTAATGCCTAAATCTTTATATTCGGGAATTGTTTCGGGTT |
| | | AGGGGGAATACGCACGAGGCTAACACAATATGCATAGTGCGTATCATTAGCGTATGGAGGA |
| | | CGAAAAGAGATATACCCAATTATAGCCTGAATGTCTTAATCAGACCCTTATCGTCATCTCA |
| | | TTTTTGACTACAATCGGTAATAACTACTCGGGTTTACTAGATCCTAACGGGATGACTCATA |
| | | ATAGAACGAATAGTGTAAAAGCAACCTACGCGTAAGACCTTCCCGGTCATGAGGATGTCAT |
| | | CCTATGCAAGCGTTCCTCCCGCGAACGCCACGTGATCTCTCGATTCCATTCTATAGGATTC |
| | | ATTAAAGCTCTACTATTACCCCAATTGCTGGGTGTTCTAAGATCTATAATGTTATTGTCCA |
| | | GATTAAGTTCTCCTGCACTACTGGCGATTGTGTCTTTCGCCCGCTTGTCCCCCCGTAATTG |
| | | GATCGGGCCTTCGCGTTCTGCTAATATTTGTTACGTCACGTCGGATAACCCCTACTTGTGC |
| | | AACATCCTGACGAATGTTGTAAAAAGTTTTTCTTTGGAAATTTGTACAGTTAAAAGACAAG |
| | | ATAATATGATTGGATGGCAAGTGACTGTAAAGTTCTATCCAGTGTTTCGTATACGATTAAT |
| | | GAAACTAAACGAGAAACTTTGCTGACCTCCACCCAAGATAGCCTTCACTCTTTCACTAACT |
| | | CCACGGTGAATTTTTTTTAGTAATTTTCATAAAGGCAAAGACTAAGTTTACCTAGTAACGC |
| | | CAATCCCCCCACCATAGTACACTGTGATTCGAAAAAAGGATATTTTTGAGCTTCTATGCTT |
| | | TAGGGATATTTAGTTTAACGGAAAGCACCGTCAGCTTGGAATATTAAACACGCACATGATT |
| | | TATGGACCCATAGTTGACATCAAGGTCTTTGATACCGACGGTTTTCGTATTTTCCAGTGAA |
| | | AGCCGAAGCTTTACAAAGGAGAGAGTAATTGAGCAAATTTCTCACTGCATGTCACAGGGAC |
| | | TGATAAATTAGTCCAAAAACTTTATTACGTTTGACCTTAGAGGTACCCTAATGCGGCTTAT |
| | | TATTTGGAGGCCAGACTATTGCGCGTAACAGGCTGTTTGAGCATCGGT |
| 283 | 38.20% | CTCCTCGAGCTTATAGAAAAGTCAACGAATGTGTAGAACCAAGAAAGTGACCAGCTATCAA |
| | | ATAAATAACAAGTGAGAGGTACAGCGTATCTAATAGGCGAAAGTCTAGCTCCAGGTATCGG |
| | | TGAAGTCTAACTATGAATTAAACGCATTGCGTAGCTACATGGTTTTACACGCACCATTAAC |
| | | AGGCGCATAACTACTGCCTGAATCGCTCTGATATTAAAGTCAAAGGAAGCTAAAGACTTGC |
| | | TATATCGTTGCATGGTGTTAAGTAAATACGACTCGAGTATTTTAAAAAATCCTCTGAATCG |
| | | ACCAACTATTTATTCGTTCATTCTCTGTCATTGAGTAGCGCTAATCAATGTAGTATTTGGA |
| | | TCAATAACCCTCTGGGTTAGGCGACTACATGAGTACCCTTGGAAAAACTCTGGTCGAGCAA |
| | | AACAAGACACATGGGGTTTAATAAAGTCTATACAGTTTATAATTATGCAAATTTGACGAAT |
| | | TTTGTACAGAATTTTATCTATAATCTTAGGGGGGTATACATATGACAGCTTTCCGGTGTTA |
| | | CAATACTCCTTGTGCTTTGTACACTTGGCGGAAAATTCACCACAATGTATGGGGTTCCGCG |
| | | CAAGCTCTCTTTTTCGGTAATCTGGGATTCCTTTTTTGTGCCCTTTTACATAACAAGACGA |
| | | ATTGGTCTCCTTTTTACTCAGAAAGAATTATAATACTTTTCTTACTTGTCCGTTTCCCCTC |
| | | ATCTTTTTTTACCTCCAAATCCGATTCATCGCCTTAAGTCCAGTGTCTTCCAATGTAGTGG |
| | | TTTAACGCGAGCTACATAACCATCCCGGATGTATACGATTCTACAGCGTCTTGAAAATATT |
| | | ATGTTTAGGTTTCGGGTGAAACGCACCTAGAAATTATAGCAATAATAATCTTAAATCTCCT |
| | | CATCATAATAGATAGGTTATTGATAGGCGACATGAAACCCAGCGGATTCACCTATCACCAA |
| | | TCAAACCACAGTTCCTTTTGATGCAGTCATTCCTACAGGCATCCTATTAACAAACAAGCGT |
| | | GTGCCGATGAAGAATTCGTATCTGTTAAGCATCCGACGGCACATGTGCAAGAGTCGATCTC |
| | | CTGATACCAATTTTAGTACTTCTCCTCTGATTAAAACAACTTCCAAAGTTCCAACAGATGG |
| | | AGTATAGATAATCAAGTTTCCAGAATTAATCAGTAATTTGACAAGTGGAAGCGCTAGAGGA |
| | | CTATTCCCGGTAATACTATAACAAGTAATAGTGACCTTGTGTATAAATAGACGTTGATAGA |
| | | TATATATACACTTCTTGATAGCTGAGGTAGACGTTGATACAACCCGCAAGTGAGTCCATTA |
| | | CCTTAGGCCCTACGAACATGCTCAAACCCTTTTATGCTTTCCCAGACTCAAAATCAATACG |
| | | TAGATATATTGTAACCGTATAGAAAAGAGCTTCTGTTGGATACAGTGGTATAACAGCTCAT |
| | | GTTCAAGGTTTATACGGTATGACAAATGTGATTTTCTTTTATGTGAGATAACCGAACCAAT |
| | | TTCGAAAGATTACTACTAGTTGAAATACCAATTTTAAAGGTATCCTTTCGATTAGACCCCT |
| | | TATATTATTCTACTGTATTAGCAAATTTTAGAAAGTTCGTGTGGTACTCAAATCCGATGAA |
| | | ACTATTCACCGTGACCATTAAATAAGTTTGATGATCACCGAGAATTCACACCTCGTAAATA |
| | | ACACCTATCTTAATAGAATTCGTGCGCAGCTCTAAGAGAGAGCATCTTCCAAAACGAAGAG |
| | | CTGTTTACAATTGCTGCCACGTCTTTGATATACACTCTTTTATTGTCCAATCCGATGTTTC |
| | | ACAATAGGATCCATGGTTCCGGTTACTTCCTAGCTAAAAGGGTTTGCCCACGCGGTGAGGG |
| | | AAGTCTGTCGGTATATTAGACGTAGTGTTCACGAATAAGTAAGATTTTTAATTTGGAATGG |
| | | TTTGCAACAATTACATAAGGATAAGTAAACGCGCCGTATAATGCTCTA |
| 284 | 40.00% | ACATATCGTATGAATTCGTCTAACATTTGAACGGACCACACCATCTGATCCGCACTCAATG |
| | | GACAGTAGGCATTCGGTTACACTTTCGTCTGGAAGAACAGTCCGAATATGAAAATATGCTT |
| | | AGATGATTCCAAGTTAATTTCGTCTATAAATAAGTAGCTTTTGCTCTATAAAGATAACCTC |
| | | CTACAGTCGTAACAGAGCTCATATACGATAAGAAGAGTATACTTTTAGTTTTTCGCACATT |
| | | TAGCCATTCAATCGAGAACATAGACGCCTCGAGCCGAATTGCTTAGCACATTTTCCTAATA |
| | | AATGTATTCGAATATCCAAAATGAACTTGCATGACTCCGTAGCACGCACTAGATTTAGTGT |
| | | GCCTAAAGATTAATATCCCAAGGTTGGGCTAGAACTAAAAACGCTGTTGCCAATAGGTTAG |
| | | ATTGTAAACTGGCCCTTAACAAGCTGATTATCAGGTGCTTTGGATACTTAGCACATACTTA |
| | | ACACATCGGCGTGAATAAGTGGGAAAATGTGCACAAACTCATTAGAAATTCTGTGATTGGG |
| | | TCTTTACGTTATGTTAAAGTTGGTATTGCTTATAATAACTTATTCTCGCAGCGTACTCGAG |
| | | AACGTTTGAATTCGTGAGAGCCCTTAAATCAACGACCCCCGGCGTTTAGAAACGGCAATCC |
| | | ATATACCTGTCATAAATTATCTTAGAATTATTATTATACCCTAGCCTTAGCCATTTTGTTT |
| | | ACCAGAACACGGATGGATCTAGTTACGATTCATATAAAGTGAGAGAGGCTAGTGTTGTAAG |
| | | GGAGTGAGAGAGCTTGCATCTTACGAGCTCTTAGCTCCTCTTATCAAAATATCATTTGGGC |
| | | CCAACAACGCGTAAGTCAGATGATCTATTAGCAGTTTGGATATGTTCAAGAAGTCCTCCAG |
| | | CGGGTTTGCGAGATTCTCTGTATCGTTGACTTGTGACATATGATTTGTATTCCAAGACGGT |
| | | CAGTTGCAATCTTGCCTGAACTAGTTGGATTATCAGCCACCCCAGGCTGTTGCATCTAATT |
| | | AAGTTTTCCTATCTGTAAAACCTTTCACTTAGCAATGGCTTAATGCTCTTACCGATCAGCT |
| | | GGAAGCCGGTAGTACTGTCACTTGGTTTTCTTAACCTATCAAAACGGAAACAAGCCGTATT |
| | | TTTGATGGTAGCACTTCAAATGGTGGGCAACCGACTAAAGAACGTCACTCTTTAAATTCTC |
| | | ATAAGTTAAAATCGGATGTCGAGTCAATATTTTGTCGGGCCATGGGAAAGAGAGCAGTATG |
| | | CTACCTTCTTAATCTCTACCTTACTTTAGACAAGCATACGTCAACAACTGTGACTCTTCAA |
| | | GGACGGGTATTCCCTGACTCAATGCTTTGGAAGAACATTTAACTGGGTTCCATTATAGTGG |
| | | TCGGACTCTTTATGCTTATGTCGCACCAGGTCCATCTATCGAATTCCTGTATTCTATAAAC |
| | | ACCGGCTGCACTCTAAGAAAGATCGAGCTTCTGATTCCAAAAGTCTATAAATGATCAGTTA |
| | | GCCTAGCGCCGACACATTGCTCCGTTAGAAGCTTGACGTTTGTTATTATGAGGGATCACAG |
| | | ATTACCGTGTGTCGATTGGTGGCTCACTTATCTATGAGCCAGTTTCGTTATGGTCATACCT |
| | | TTAATTAAGGGAACATCGTGCTAAAATTTTTAGAATGGGGTACTGTCTAGACTGTCTCGAG |
| | | GATTCATGCCGATGAAGACCTGAAATTTGAATCGGAACTTTTGTGGCACCGCCGTATCGCA |
| | | AAATGAGAAAAAGATATCGTTAACCCCTTATAAACCGCAACTAACTAAGTCAAAATAAGTC |
| | | GACGTGACTTAAGATACTGATTAAGAAATGGTATCACGGCTCTTTTGCAATACCATTACCA |
| | | AAATTGCGAATGAAACTGTTTTGGCCTATCTTAAGCCACGAATAATAT |
| 285 | 40.40% | ATTCTTAAAGTCGATTCGGTGTCATAATAGGGTTATCTAACATATGTACAAACGCCCTATA |
| | | AAGTTATTATCGGACTGGTGCATAAGTAACAGTTCGCTATAAAGTTAAATGCTATCAAGAG |
| | | AAATAAGGCATACTGTGATGAAAACGAGGTCGTACAGAAACACCTGCAGGAATTAATCTGC |
| | | CGTATCATACAAGGAATATCGTTGGAGTCAAGATGACTGCCCATTTGCAGTTGTCATCTTA |
| | | ACTGATGATGGTTTCTTGCTTGATAGCACCCGCCTCAGTAAAAACAGATGGAACACTCCAA |
| | | TGCTAGCCAACTGAAATTTAACGTTAGTACCAAAGGCATCCAAGCAGTCCCCTGGCTAAGT |
| | | TGGAGTGTGGCATCGATATAAAATAGTTAAAAAAACGGTCTGATGTTTCATGCAGTCGCAA |
| | | CCACGCATACGGTTCCGGTTCGCAACGATTGATGTGGCGGTCTCAGTATTTTACAAGTTTT |
| | | AACATGTCGGCAGCCGCTAGGTAGATACCTGCACCCTGTGGTTTCGTATATAGGGAATTTC |
| | | GGTGCTTTAAGATAAGGATTACTCATAGGGGATATTACTCGATTGCCTCGAAAAATGCGAT |
| | | GAGTCTCTATATTCAACGGTCTATTACAGGCTTTCTATTTTCTCGGGACGCCTAGGAGTTG |
| | | AATGATGCACATCATTAAGCTACTTATGCGGTCTTCCATACCATTCCAATGTCGTCGAAAG |
| | | AGGATGCAGTGACAACTCAGGATACTAATAATTCCTTGAGAACTGTCTATTTCAAGCCTAT |
| | | TCTAACATAATTAGTTGCTAGCCATATAAGAAAATATCATCAAACAGATAGGGTTGATAAC |
| | | AGAGGGTGCTGCCCGTATAGTGAACATCGTAACCGGGTTTCACATCCTAGATTGGTGGCCT |
| | | CCTACTATGTAAGATGTAGTTATACTGAATGTGGTGTTGTGATCAAGACGTAGGAAAATTT |
| | | ATCAGATATGCCAACTAGTATCATCCTGAGTTATAAAGGGGGTAATTTCGGACAAAGGTGT |
| | | TGTTTCAAAAGGTTCAAGCCGACGTACCCGCACATCAACTTATCTTGTAATGATTCAAGGT |
| | | TTATGTAGCTTGATCACCAAGCAACCCAAGCGAGCTGTACCAGATACGATTATGTTAATAA |
| | | AGGTTTGGCGTACTAGACTTAACGCTAAGGTTTCGTAATGTAACGCCTGCATTCACGTCAA |
| | | TAATAGCTCAGTATGTGAGAAGTCCGATGCTGTTAATTCTAATAACGCTCCCACTTGAAGG |
| | | AGAAAGCGGGAGTAGGTGCGTTTGTTCAGAAACCACTTAAGCGGTTTGTTTGTACGTACAA |
| | | AATTTGCTTTTAGATGTATAGTTGTATACATAACCATCGTCCGAAAGTAACCTTCATATGA |
| | | AACTCAAAGGCATTAGTTGGGAAGCAGTATGTGGCGTTTGTGACACATCGGGAATATAAAA |
| | | TTCCAATATATATTCTAAGTAGCAGTTAAATGAACTCCACTATGGTTAAATACTTGTACCT |
| | | ATCGTTATTCGCAATTGTGCCACTTTTACATAGATTGTGAACCGGTATATCGCGTGGTCAA |
| | | GACCAGGCTTCAAAGCTGTAGAGAACTGTTTATTCTTTGAGTGACATAGTATCGAGACTTG |
| | | TATAAACATGGATGGTACACAACGTTGGAAAAGCCGAAAGCCAATAAGATATTTAAGCATT |
| | | ATGCTTTTATGTCAACACTGACTTTCTAAACCACACACCTTAAATCAGTAGAACAGCATTT |
| | | TGAAGGAGTGGCTAAACCATGTTGCGTGCAATTCTCCGGGCTCGTAAAAACGTGTCGTGCT |
| | | AAAGGCTCTAAATCTCGCAGTAAAGGAGGCCCTCCAAACTAACTTAACTCATTTTGACGAA |
| | | CTCAAGTAGCTTCTATTAAATTCGTCCGAATACCATGAAGAACGGGATTCGCATACTGCGT |
| | | TCGCCGTAGTGGAGCTCGTTACAAATCAAATGGATCGATAAACAAACG |
| 286 | 42.30% | TTAGTATAGTTAAGATAATGCGTCGCTAAACAACATAAAGATTCTTTACCGATGAGTTCTC |
| | | GCTGGTATTCGCTTTTTTAGTCTTACTCGCTCAAGTTATCTTGAGAGATGTGGAACTGAAC |
| | | CACTTGAGGTAGCCCCATCAATTATAAGGAAATTGAAATAGGATCGAAATATTCTGAACTA |
| | | TTTCCATCTAGTCTACTGAAATTAACATTGACACCTTTCACAAACGAATGGCAAAAAAGGA |
| | | CGGATCCATCCCCACAGACAACTTCGTTTATTTCAGCACATTTGTCCCTGGACAACAGCCG |
| | | TATGTGGTTCGACATACTACCTGATAGTGAGCGGTTATCGAAATGTCCTTGACTAGCTACT |
| | | AAGAGGCTTTATACAATATTCCTACACACATAGACCCAGTAGATATGAGTTCTAGTTGGAG |
| | | ATTTTTCAACACAATTACGCCACGAGGTCCGACAACGTATCCTCCACAGTTAGGAACATTT |
| | | ATTACAAGGAGGTTAGCTCCGTGCTACAGCAACACGAATTACTCCACCGTGTTGAGCAGGT |
| | | AAACGAGGGCAAAATACACCCCAAAGCGTAACTGCATACGACTTTCCGCTCGAAGATTGTT |
| | | AAAACAAGACTGCAATTTCTGTGGCAAAAGACACTAAAGATGACAGTACAGCACCCATGGA |
| | | GAGTTTGTACCCGGTTCGACCTAAGTATCTGTTGTCCAGAATCGTGAAATTTGAAGTGGCC |
| | | TAAAAGCTGAGACGAGTATAGTAGGGTGGAGGTTTCCTATATGTTGGTCGGTCAGTAAATA |
| | | TTTAAACCACGGGAGTTAAACTTATCTTAAATGTATCTATACATTAGTATATAGGCTGAGA |
| | | TTCGATATATATAGACGCCACCCCGAGAAATAGAAAGATAGTGATTCAAATTCCTAACAGT |
| | | TCGGAGTGGTATACGCATTTCTGAGTAATTTGGCGTACAAAGTTTGAGTAGAGCACAGAGT |
| | | TGATAACTAGAGCAATGTCTGAGAGTGGATTAACTTGGTGTGCTCTGCTAGAAATCCCCAG |
| | | TGATGATCTCTCATAAAAAGTGACTGCAAGACTAGGATACAATTTATTATCGAAGTATCAA |
| | | GATCGTGGGTTCCTTTTTTCCTGGTCAAAGATGAATCTGTCTTACTTAACGAAACACAGGA |
| | | ACTTTTCTTGCATAGGCACCGATCTTGCTATGTATTGAAGCTACTTCAAAGGACCTATCAG |
| | | CGGGTGTACACAATGTCGGAACATGCATAAATGGCAGAAGGCGATGAGTCATTTCGCACAC |
| | | CAACAGGCCGACGAGCGTAGGAGCGACTCAGAACACTACCAACTATAGCATAACGATAAAC |
| | | GGAGAACGTCCATGCCGTTATGTGACCATTCGGTTCGGAGTCGTGGGTTACCGACCACGAT |
| | | AGAACATGGCACACTGCTTTCTCACTTCCCCAATAAGAAACACCCTGGACGTATACCTCGA |
| | | TTGGATCTGGAGACAGTACTCGGATCCACACCTAAGTAGTACCTCACTGTGGGCGATGGCC |
| | | AAGACGCGAGGTTGACTATCTGCGTGGTGGAAAAGGCCGACAGATCTTTATCAATTGTAGT |
| | | GAGCTGATGAGTCCTTTATCCGTTATAAGCTACTTTTATTGGGTAATAGATGGTGCTCTTA |
| | | CTCCTTCGAGTTAATATATAGAAATCACCGCAAAGTTAAACGCAACATGAGTGGTTTGGAT |
| | | TAACAACTTCTGGAATCATTATAACCTTAGGAGCGTTCTAGTGATGCTGAAATTGAGACAG |
| | | TAAAAAGTGCCCATGATGTAGGAAAGTCACTATAAAGTGAATCTCTTGTCCTTAAACATAA |
| | | AGCGCGGTAAACACTCACGTTAAGATGGTTGTGGCCACAACATGACTCTTGTGGTTCTTGA |
| | | CGTGTTAACGCGGTGGCACTAGCAGGGATGATACAAGTTGATGCTTACCCATATGATTATT |
| | | GTTCCCCGGAGCCACCACTAAGCCACTAAATGAAGATTTTTGCGGCGA |
| 287 | 38.20% | GATGTTCTGAAGTTCCTTAGCGTACAAACACAAAACGTGCATTGGAAAATGGAGAGGGAAC |
| | | CCTCTATGTCTGATGATTTTTTCGGTTGAGCTAATTCCAGTGCAATCGACAATAAGGGCAT |
| | | GTCCGAAATTCGCTTTTTAATGGTAGTAGGTCCGGCATCATTATGTTGTCGGCCTAAATAC |
| | | CATAATCATTGCTCAACCTTCAACTCTTTGCTGGAACAATTAGTACTTTTCGTTTGCGCTT |
| | | AACCATGCGTATAATGTAATAAAAGCACCAGTTTATAGATATCGGAAAATTTAGAGTTCAT |
| | | GCCATAGTTTGAACCGACGGTAGGTACCTATAACGTCTTTTGATTTCCGCAACCTATGTAT |
| | | TGTAAGCAGTTGTCCTAAGGAGTATTTTCACTGTCTAAGTGGTAACCAGCGGCGAGAACAT |
| | | AGTCGGCGGAACGGTTCTGATTTCGACTAGCATCGGCGACATTGCCTTGTCAATCTCCATA |
| | | ATGATATAAACATGGTCTTTTAACTCTCACAACCTAAATTATTAACAGGTCGATACTTCTC |
| | | TGGCGAGGTTGTTTTAAAACTTCCACTCCGGATAGGAATTTCATTGAAAATATAAAAGGTT |
| | | GATGTGTCAATCGAAGTCTAAAAAGAATGAAGATTAGTGTCGCCTAGGACATCTATTTGTT |
| | | TTAAAGTGCAAGGAACGTGTTCACGTAGAATTGTGAAATTGGATACATGTTTAGTGTCATG |
| | | CATTGTTTATGGGATTGACTATAACTTAGATAGAGAACTAGTTACCCTTATTACTTTGCAG |
| | | TATATGAACGACTGATTGTCAAGACTGAGCCTAAATTAAAGTAATCAGCACATTTTGGATA |
| | | TGGATAGGAGCTCAGTTTCTGGTTTCACTCTCATCGACTTCTTTGTCCAAATACGGCAATC |
| | | ACGTAATGCATAAATATTCAAACATAATGTGATGAAAGAACATATCACCCGTCTAAAAAAT |
| | | TAAATATATACTATAGTGCTGCAATACATCCTTAAATTGTCCTATATTGGTAAGTCAAACG |
| | | ATACAACCTGCATTCTTGGGGGATAACTGATGTTTACTGGACGGCGGAAATACTTTAATTT |
| | | ATAGGCTACTCCAGTGCATAGTAAGAATCATAATTTGGTAGCGCCTAGTAAAAAGAAATCC |
| | | TCAAAAACTAAACGCTATTCTGATCGCTATCATCAAGAAATGAATTGTAAGTGAGGGCTGT |
| | | ATTCTAACTCATCCTAGCAGGATTTATTGCCTGCATCATCGACATTCTGTTCGAAGCGGTG |
| | | ATCCCCATTTGGACAAATTCAAGGTTTGGATTATCTAGCGCCCTTGGAGTCTCTTTACGTG |
| | | TTTAGGTGTTCCTGTAGGAAAATCATCTTATTGTCGCGAATAGAAGGTAGAAAAAGACCTC |
| | | AAAGTTACCATATGCACCATGGAGATGAAACGGTAAAAGTAACTGGGACCAAAGCTGTCCT |
| | | TCCGGGATTCATTATTACCATAATCATTAGGCATCAATAATATTCTGTGCGATATGTTGCT |
| | | CGGCTTATTAACCTCAATGAAACAATATGACCGCATATCGCTACAGTAAATCTACGACGTT |
| | | TTTACTGATTGATTGAATCGCACTTTTTAATAATTGTATGCCCCGATACATAAAATGTCAT |
| | | AATCGAGAAGCATATAGTAGTATTGTAGTATCCTCAGGATCGGTTGGTAGCTTTAATACGT |
| | | GTAAATTTTTCTCGTAATTATCGAGAGTGTGGAGACGTCCGTGTACTGGATTCGTAAGAAT |
| | | TCAATACCCTGATGTCCGTCCGAGTAGATCGATAAAGTAAGTAGGGATATTCAGATATTTA |
| | | ATGTATTTCCTGTACACTGTGACATCTCTGCAACGAGATTGTTATACTGGCGGCGCGTAGG |
| | | AAAAATTCAACCAGTCTGTTTGCAGGGATAGTTAAAATTCATTAGAGACCAGAGCAAATAA |
| | | TGAGCATCCGAAATGTATCCAAAGCGATATACGCGCTTACAAACTCTG |
| 288 | 39.70% | TTGATGTGCGAATATAACATTGATCATCAGAGGCAAGGTGATAGGTATTAAAACGTTAGCG |
| | | TCCACGCTCCTGGTTCTATAAAACTTCTTTAGATGCTGCTAAGTCCATTGATTTACTGTTT |
| | | TATAGATACGAGAGTAAATATAGTTTAAATTTTTTAAGTTTGAAATACGTGTAGCTATCGT |
| | | TGCGCTAAGGAGAGTTGTCTATGTACTAGTGATTTCAGTCGGAAATAGCAGAAACATGAAC |
| | | CTATCACATGACTGTCGAATGGAAAATTTGGAGTCTGGAACATTCAGTATGAGATATACAT |
| | | TAATCCATGACTCAGAGGAATTGACCCACTAATGTTATTCTTAGTTGCAATTCCAGGTATG |
| | | TCTAGAATTTGCAATCGGTTAGCCGTTGTGTACTTCGTATCAATTTTCAAACAGAATACAA |
| | | AACCACGCTAGTTAGCCGAAATTACTCCTAATTGTCGTCACTATGTAAGAGATTTAGAAAA |
| | | AATAGTATTTGGTACTACTAAGATAATCGCTGTCCACTATAAACTTGTAGGTAGTTAGTCG |
| | | AGTGTTCTGCAAGGGTACATTCATGGAATTCGCGAGCAACGTTCGCTTCTCCCCAAATATT |
| | | GATATAAAGACGATCCATTCTATGTATTTTCGCACTAGTAAAATACCTATCTACTCGACTT |
| | | ACGCTATAGCTCAGGGATCTATTTGTAGGCATCCACAGCTCAGACGAAATAATAGATTTAC |
| | | GAACTGATAGCGGCCCTCCATGCCTGCTAATCATGTTCATACATCCAAACAAATCGTTTTG |
| | | TTGGTAGACAACAACATAGCGATAATTTCAACTGGTTGAAATGGTTGTATAGCTGAATATA |
| | | AACGATCCCAAAAAATTCAAGATGGTGGCTGCACCGGAACGACGTTAATAGCGTGAGGAGG |
| | | TGTTAAAAGCAACAAAATCACACCCGCCGTCTTCTAGGGTAAGCGGGTGCCAGCCGGGTCT |
| | | ACTGGATAAGTAGATATTTAGCAAAGAACCTCAGTTATCCATTTTCTGGTTACGTGCACAA |
| | | TTAGTTTTGCATCTGCCGGCTTTTGTCTCTGGCACTTGACAAACCTAGCAAAACTCAACTG |
| | | AGGGGTTAACACGCTCTAAGATTCCTCTTACTAGATGAGGTATTCATCTGCGTATCTGATT |
| | | CTACGTTATAGGCTTTTTCTCTCGAATACTAATGTCTGGACTGATCAATAAGAATTGGCTA |
| | | ATTGCGGAAGTCAAAATAGAACCAATTATATTCATACTTCTATTATTAGTTCTAGGATGAT |
| | | TTTCCCGACCATCGGTAGTAGGAGGAGGTGATGTAACTCAGTAGTATTATGCTGAGTGATT |
| | | GCACCTCTGATTCTATTAATATGGGGGGATGCTGCTTGCCTCGTGGGTTAGTGTCCGGATG |
| | | AAAACCCCCCTAACCTATTCACGTATAGTATCCCAGTCAATTGAGTCAGTGACCTTAATCC |
| | | TAACAAAAAATACAGAATGCTGTGAATGACCTCGTTCTTCTTATTGTGCACGATCTCATTC |
| | | GAAAATGAACGGTATAGAGTCTGAGCATCACGATATAAGAGATTCATTCTGTATTATTTAC |
| | | GAAAGGCGTAGCACCATTCGATCAGCGAGCAGAACCACGGGGCAGTATTGAATTTCCGTTT |
| | | TTCCGATTTCAAAACGGCTAGAAATGGCTGCTGGATGATAGATGCCCAACTCACACGGTTG |
| | | AACTTGCTTATCAATTGTGCGGTTCATATCAGACATAGCAGTCTGCTTGGAAGATATTGAG |
| | | TAACTTCAGCATTCAAACGCGCAAAGCTATTGAGTTGCCCCTGATGCTGTCTATCGTGTAT |
| | | TAAGTGATCGTGGGAATTAGACATACAACTTTACCTCTTCTAGCTTGTTTATAGAGCCTCA |
| | | CCGAGGTATAAATCATTAATTACCCAGGAGACCGGTTTTGCTATTACCTTGTAATGTTCAA |
| | | AAAAGAAGTGGAACACAGTGAAAGCCTCATTTCTCAAGCAAGTGAGTA |
| 289 | 40.40% | TGTAGACATTTGTCTTCAATCTAACCTCTTTCTCAGGAAATAAGGGCTTGTATTGTTCCTT |
| | | CGTTTGTTTACCGCACAGAAACAGCTTCACTTAACATACATTGTAAGTGTGTATTTCTCGG |
| | | GGTACGTAACATAACGAAACTTAAAGCAATCAGACATACAGTGCCATTCCCTACGGTACTG |
| | | TCTCAGTATGTTAATACTACTCATTTGCAAAAGGATGTACGCACTTCATACTACAGCTGCT |
| | | GACGGTGTATATCAAACAATTATATTAACGCTCGTAGGATAGTTCACGTCCGCCATATCTT |
| | | TGATTTAGGCTTCAAAATTCAGAATAATACGAAATAGTCTGTCTACTAGGCCAAAGTCACT |
| | | TAAGGGCTAAGAGTGTAATGAGTAATCAAAATAATAATCGTTGAGTCGTCAATTGGAGCAT |
| | | CAGTTATGGCATTAAAACATCTAGTGGGTCGAAAGGATCAGGAAATTATGTATGGGTGAGA |
| | | GTCGCTGCTACGGTATCGCTTTTGGATTGAGGGCTACTACACTCAGTACCCACAGTGTGTG |
| | | TATTAATAAGAATCGCAATATGCGTCCTTTTAAGTTTTAAGGTACCCTACCTTTCATATCT |
| | | AGTGGAAATCATTTACGCCTATGCGACAAATTAGAGACTTTTATTTGTAAAACATTGGATG |
| | | TTGGAATGACCCTAGATGCATGTTAAATAGCACGTTCATTAGTGGTACACGCCTATCACTA |
| | | ACGCTATGGAAAAATAGAAGAAGCCAGAACAAGTAAACCTATGGTGACAAATAATTACATA |
| | | AGGAAATCCCTCATAATTAGAATACCATAAAACGTTAGTTGTACTATCCGTAATCTACCTT |
| | | CTAGCGTGGAATAGTTGAGTGTATTCTAGTGACGCCCCGTTCCATAACGATACATGTAAAA |
| | | TTTACAGCGACGTTTAGGAACCCTACAAGGGGAGCAGCAGCGAGGATAGCTGACTAGCCTT |
| | | ACAATAAGCACCCATACTTATGATTGACATGATGGTCATGCGGCGTTACCACTCCGCTAGC |
| | | GTTACTTCTTTCGTCTTGTACCGGTTTGGCAATGCGATGGAGCCCAGGTACCGTAGAGAAA |
| | | GTAGCGATGTGTGAGGTCGAGTACTTTGTCAGAAAGCAAGTCGGATTGCGGTCCCATTTAC |
| | | CGCGACGTGCATTTGTACAGTATGACCGTTTTTTACCACTTACTGATGAGGCCAGACTAAT |
| | | AAACGATATTTGGTCACAGGACAATATTACGGCCAATTATGAAATAACTGACTGGCCTATT |
| | | GAATGACTAGGAATGTCAAGTCCAGACTCTAGCTATTTGGGAGGTTTATATGTTTGGACCG |
| | | ACTTGTGGGAGTTTGACACTACGAGTAACAAGATTATCCCTTTTTATGCTGCGCTAGTTGA |
| | | CATGGATTGACGAGGTTATTAATATCCATGACTAACTCATCACAGCTTCCCGAGCCGAGAC |
| | | GGATTATTTTAATCTCGTTGATCGATATATTAGGTGACGTGAGAAGAAGATGTGTCGTAAT |
| | | CAGTAATAGTTAGGATCAAGAGGTTAAAAGAAGCGCCTTCTTCACAGATTCTCAGTATCTA |
| | | CCAGCACAGAGTTCTCAGTTTCTAACGTGTTCCGTATGGATTTGCGCCACTTTCTGAATAA |
| | | GTCTTATGAGATATACTTACCTGGTCCAGATGTAGCAGCGAGTTAAGATTATAACTGCGGT |
| | | TTAGCACGCAGCGTTTAAATACAAATACTCTTGACTGTTATAACGTTCAGGATTAGGAACA |
| | | GGTTCCTCACGGATATAGAACCCAATTCACGTGCATGAGGTATTCTATCTTAGGGGGAGGA |
| | | ACTGCGCTGGAGCTTGAAACTGACCCTCTAGGCGCTTGCTTTCACTGAGATCTATTCAAAC |
| | | TGACGTTTAGTAAGAAATCATAAGACTTATCTACGCCGCCTTATAATTTATGTTATTAAAA |
| | | CATGATCATGCGATCAATTAGGTAAATTTCTTTGTGCCTTGCAATATG |
| 290 | 38.60% | CGAATATTTATTTTTCCTACGCACCTACACTATCGTGAAGTTCATGGTATCAATTATATGT |
| | | CACTAGAGCCACAAATACGTACTTAAATCATTTACCTCGACTGAAGGTTGTAGGCTTGGAC |
| | | ATACTCTTGCCACCATTGTAACAAAGGTAGATCGGTTGGACCCGAAATTTGGTACTTTTAA |
| | | TCTAGAATCAGCAATATCCTACGGAAAGGCCCAAGAGATGTCTCAATGGATGAGAGTGTAA |
| | | TTACCTAATTTCAGAAAAGAGAGTTTAACACAAATAAGAACAGACGAATATCAATAAAGTG |
| | | CACGTCGGGCCTAAATGAGCCCACAGCCTGGATAGATTAAGTGCGATACGTCGCTACCAAC |
| | | GAACAAAAGTATTTGGTATTATGACATCGGCTCCGACGGTATAGGATAGGAATAACTCCCA |
| | | AACAATATAATCTTGGATACGATTAAGTTTGAGTTTGATTGATCCCATCAAACATTTGTTG |
| | | GTATAAAGTTAATGTGTGATCCAGTTAGAATTATATGAACATAGTGTTGTCACGATTTTGA |
| | | GACGACCGTTAAACATTATACTGCGGTGGCATAGCAAGTTCATCTCCTGACATTAGTCAGC |
| | | ATTTAATAGTAAGCAGGAGTACTATTAACACGCTCCTATAATCGGTTGCCTGTTGGGGATA |
| | | ATCAGAACATGAAAAACTCCATATTAGAAAATTACATAATATAGATCACGTGTATGAAACC |
| | | TAATACCGCGAATATAATTACATTATGATTGCAATACATAGGGTAGACTCCTAGTTAACGT |
| | | AAACCAAATAACCGACTCGAGAAACACAGGACTAACAATTATAATTTATAAACTAAGAGTG |
| | | CTATACTAGTTACTGCCTGATACCTATGTTTATTTGCAAGTCAAAAGTTTCAAATAGCCCT |
| | | TGGCAAGCTACATGATGGGTGATTGGAGGTGGGACTAGGAGTTCCGTCCTTAGTCTGAATA |
| | | AAGAACATGATGTGCACCGATTTGTCGTCTACTCGGACGTTGTGGCAAGAATAAAAGTGAG |
| | | GTATAGTACCGCTAGCCGCAGAGATACTGCCTTCATATGCGCCGATACTCTATTGTTCATA |
| | | AACAGCAATGAGGCAGAGCACATAATCTTAATTATTAATTTAGTTAACGGCTTCCCAATTT |
| | | AGCAATGAATAAATTTTTTGAGGTGCATCTGTGATTAATTCACCCAGAAACGCTTTCGCGA |
| | | ATTACCTGTCACTATAGATCCTTAATGAATTATCTTCGTCGTCGGAACAATTATCGGACTT |
| | | TATTTTGCCTGTTTTATGTATCGAGTTAAATAACGGGAATCATAATTTTATATTACATCTG |
| | | TTTTGTATAGCGGATCTCAGTAGGTTACATCACTGTCGTCGGATTCAACAGCAACAACACC |
| | | GTTAATGAATATAGCTACACTGCATGAGTCCCAACAGCACTGGTCCACTAGAAATATATAA |
| | | TTATACGAATACTTTGCTATGTTCATGACCTGTCAAAGGAGAAATCTAGTAAAGACCCACG |
| | | GATATCGAAGAACATTGTAGTTCTGACTCGGTTTGAATGTCCGGTAACTGCAGGTTCCCGT |
| | | TATACTGAGCGGTCCGAAAATGGCAGTCTAAGTCCCCCTACATGACGATTGCTATTTATTA |
| | | GGTCTCAGAATATAACATTAGACACAAGAGCACAATAGTCGGAGTATGCGTTATCGAGACC |
| | | GTATATGAGTCAATCGAACGTAGATCGATCATAGCTAACTAGGTGGTGTATCACTGACGAC |
| | | TTGACGATGTTTTATCGCTGATTAGTTTATGATCTTGTAAAGATTGGATGCTACATATTAT |
| | | GGTAATTTTGCTACTTCCCCCAACTATACCAAATGACTCACTGTTTATCAAAGGTGACTGG |
| | | ATAGGCGCTAGGTATATCCCGGTGCGCAATTATTGCCCTGGCGAGCCGAACATCTCGAATA |
| | | TGTAAAGACGAATACTCCCTAATTACCTTTTCGAGGTAACAATGAATA |
| 291 | 42.20% | ATCGAGTTGGTTTCTACGAGTAGCTGGCAAGCGCACATAGAACACACATTGCATGTGAGTG |
| | | GAGCGATTGCGAGACGAAACAACCTTCCAAAAGCCCAACGATTACAGTGCTAGTTATCTAT |
| | | GGGAACTTATTCCCTTAGGGCCAAAGTCCCTAGGTTATTCTATACGACTCACACCGAAGAG |
| | | GCTGTAAATTAACCCGAATATAGATGATTAGTCCTTTGTTTGTCTTAGGGATGGCACCATA |
| | | ATAAAATTGTC&AATTAGGGTACAGGACTAGTTCGATTTCTTCTATCCGTCGTCCTAGGTT |
| | | TATATGTGGCCGTCACCACTGTATCACATGCCAGCTAGCAACAGTATGATGTATAGCGGCA |
| | | AATCATTCGTCGGGGGCATGCAGAACGTCAGTTAACTTTAAAGATGAGACTACGTTTTGGT |
| | | CACAATACAATGACTTAGACTCATCTCTTAACTCAGACAATCACTTTTATACTTAGTGCAA |
| | | TGTGTCACAGCCACTTTATGGCCTAGCTAATCCTTATAGTCGGTAGCTAGCGAGTTATAGA |
| | | ATCTTGTTGTGGATAATCCTGCTCAACCTTGCCTGGAAGTCTAAGACCAGTACTAGAAGTT |
| | | AGGCGTCGGAGTCTGTGATGCTTAAGTTGTTCGGCCAACTAATTAGGGGTGTACCTCCTTG |
| | | TCTAATCCTCTTAGATATTATTCGAGAAGGGTACAGTACCCCTCACAAAGAGAATCTAAGT |
| | | TACCGTCTGAAGTCTGAGTGATCCGTTTTGAGGTAAACAGCTGTTATACATACTTACAGCT |
| | | TAGTCTACATGACCTACTAAGCGCTTCGTGCTCCTTACCGTCCCAGAATACCCATGGCTCG |
| | | CGTCTCCTGCCGTACAATACGTAGATTTAATACTCGTAATGTTTACAAAAAATGGCTCAGC |
| | | GAATATGAATACGATATACAGTACCATATTTATGGATACAAAATTTGTGGCATCCGCCTAA |
| | | TAGGGCTTTCCTCAGGGCTTACTCCACATACTGTTCAACCTTCTAGGTTCAGTAAAAGTGG |
| | | AGACCACGATGCAGTGTCCTTCTTAATCTGGCCTTATTTGTCGATCCCTTATCTCGCTAAG |
| | | ATTAGTCACACGACAAAGAGGTCGTTAATGACGTATCTAGCCACAATCGACAGTCTTCTGG |
| | | CGAAGATATCTACAAGAGTCGTTGATTCGTCACTTTTAGCCTTGTAAAATTGCCCTTTGAA |
| | | TAGGTGACACCCGAATGGATTGGTACTTTCGTAATTAACCGAGACTTTGGAGAATTGTCTC |
| | | CGGCGTTTCATGTGGCGAAGAATAGAGGTGACTTTGATGGCACCAGAATCTCACTGACAAT |
| | | TGCTATAGACCTAATATCGGATATTTCTGCAACTTCCTAATCGAAAAAATTTCTACAAACC |
| | | AGTCGCAGCCTTGAGTATTCGCCCTTGACATAGATTCACAAGATTGAGTCGCAAATGGTCC |
| | | TATGATAATGGATGTGTTATTGCTGGAACTTTATCATGATGCAAAGAGGTTATAATATTTT |
| | | GTGTTAGTAGCACACTTAATGCACGCAGAATCCTTAATCAATCATTAGCTGCTAATGAGAA |
| | | TGAACCGACCGTGTTGGTGTTACTGGAATTATATTCAGTATCGCTCTGATCTTAAGGCCCT |
| | | CAGCACCTGAGGTCTAACGAAAATTTTTTTAAGCCCATTCTCGCAAGGCCACAACCATCAG |
| | | TCTCTCGAGAACGACATTGGACCTCATATCCAAGCCTCCGGTTATTCACCGATGTATTTCT |
| | | TCGAGTATCTAAAATCTGCCAATACGATTCAAGAGAAGTTAGTATGCGGGATCATGTAGCG |
| | | TACCTTTATATGAATAAAACATACCTGGTAGATGGAAACTTGGTGACCCGGGAGTACGTCA |
| | | TTCTGGTACTGATACTTGAGGGTGAACATGGTGCGTGATTCCAGTATAGCGGTGAACCTAC |
| | | GACAATATGTGCATGGCATTGCTTATTTGGTGTATCGTTTTTTGAGAA |
| 292 | 37.60% | TAACTATATGGTGTCTGTTTACTACGATTGCATTAAGATTTCTAGCAATCTTCTCCAGTAA |
| | | CTGCACTTCCCCATATTGTAGAAGCGACTTATGGAGCTAATCTTTCACTTGGTTTAATGCT |
| | | AACTGGGATTTGAGCACGTAAAACTTAACTCGGACCACTTTGTTGACATAATTCCGCTGCT |
| | | TATATACCCATATTCATGTCTACGATTATAAAGTTCTTCGTATTTGGCTAAGCGTCTCTAC |
| | | CTAGGCTCAAGCCTTTTTAGCCAATCTGAACGCTAAACGGGTGCTAGCCTAGTGATTATTT |
| | | AATGACGATTTGAGTTCATGGACGAAATTACATTATTACTGTCTAACCGGACAACGGGCAC |
| | | GTCACAATAAGAAGGGTACAGTTGGGATCGCAGTTTATTCATGCTGTATGCCAATTCTACT |
| | | ACCTCTCGTCATCTTAATTCATATATAGCTGAAGGGCTAGCAAGTAGTGGATGACTATAAT |
| | | CGGGATTTAGAAGAGTTTTTTCCTCGAACATTAGCCTTATGTGTCTATTTTGTTAAAATTG |
| | | ACATGCTAAACGATAGCTATTAGCTGGAGGAATAACATAATGTTGTAAAAGGTAACCAGCT |
| | | CATCACTTCAGGAATCTTACTTCCTACGATGGCTGTCTTTTAGTCGACGTAAAGAAACCCA |
| | | ACCAAGGAATACTTAGACAGACAGGAGATCATCCTACAAAGATAGTCGATCTTTTATTTAG |
| | | TCCAACGCTTACCAATGAATAGGGCTGTCTGAGACTCAAAATATTGGACCATGGGTTTCGC |
| | | AAAGCGCAAACGGAGAACTATGATTTCTTGTTGTGGCAGCGTATGGTCCCCACGGGTGACT |
| | | GTACAATCACGGAGACTTTTATCATATAACGATAGTACATTTATCTGGATACCGGATCCTT |
| | | CATTTCTCGGAACTCTATACTTACTTTAATTTAATGGCCCGAAATCTATTATCCTTAAATT |
| | | ACACCGCCGTGGACTCGGAATGAAGATGAGTCCGGAAGGCATACTGTTAGATCGGCTGAGA |
| | | TATTGCCTAGTGGAATCGATCTTTTGATGGTATTTGTGTACATTCTAATTCGAGGCGAAAC |
| | | TGTCAATAAACTAATGGGAAAAGCAAGCATATCACGAGAAATATTCTAGGGGATAACATTA |
| | | CGTTTTCGGAACACAACAGGTTCGACATAAATCTTTTATCATATTATTTGCTTACAATTAT |
| | | TTAGGGCTTCCGCCCATACTCAGTAGTTCAAATGATGCAAAGGATGTGGTGTCTAGTAGAT |
| | | CTCTTAAATTTCTATCGAATGGCGTAGTTACATTGCAGTTATTTTTACATGGCAAAATGAT |
| | | CAAATTTGTACGCAATAGCAGTAACATATTCTCTGTAGTCTATATCTTTATGATTGGAGAC |
| | | TGTTAAAAGCTGATATGACTAATCAAGAAAATATCGAAATTTGATCTACGACTTAACATTT |
| | | TAACTAAGCAGACATCATAACGTTTATTCTTCAACGGGCCGTTACTGCTAAACATTAATCT |
| | | AACGTAAATCGGAACTCTGCAGAGTGCCCGTCTCTTATTTTGTCTGAATTTTAGAATTTAC |
| | | AAGGAGATGCTCAAGCCGAGTTAGAAGAAGAGAAATATAATGAATCCACCGAGTGTATGTT |
| | | TATACATAAAGAACTATCTTTAGGCGACGTGCTAGATCCCACTATGTTCATGTGTAACGCA |
| | | TTTATTGGTGGAACTCTCGCAAAATCTTACATTATTTCGCCATTACGTCTATACAAAAGCT |
| | | AGATCCGTGAAGGGTCATAACCTCCTTTAAAGGCATGAAAGAGGTTATCTAACTTATGATT |
| | | CTATAAGATCGTCACTGGTGGAGTAAAAACATCTGTGATAAATACTTGTGATACTCTCTAA |
| | | CATCCCTGTAATATGATGATCATAACGCTTGCACCTATTAACTTAAAAGAAAGTTGTCTTA |
| | | TGGTGATTCTTAAATAAAAGTGCCTGAGCCACCTTGTGTAATTTTTAA |
| 293 | 40.20% | CACAATAGTATAGGGACGTCTATTATTGAAAATTATACCATGTGGACATATTCTGGATTTG |
| | | AATTTATTTTTTACGAACTTACTCGTCTCTTTGTCGAACTGATCGAACCATGATAGGCGGT |
| | | CCATACGTGTAGTGTGTGCTAGAAGCATCTGTACTTGTATTGAAAGGAACAAAGTCAACCA |
| | | TGCTGTTCACCAATTTGATACGAAGGAATGTCCTATCTAACCGGGCTTATTTTACAGGCTA |
| | | AGTAGGTGAATAATGACAGGAAAAATTCGAATAAATCAGAAGAGTTTTAAGTAAGGCTCAC |
| | | TGGTCGAACGGTGATAATACTGGCGGCAAGTTCTATGTAGCTTATTAGATAACTCTTCGGG |
| | | TGAGAGAAAGAGCTTATAAATGTGGCGCTGAAATCCGATGCCAGCTGTAGCCGAGTCGCGT |
| | | CATCTCCTAACGGATCAGTTAACATTATGCTTACTGGACGTAAAGTGGCTTGTCTAGCTCT |
| | | CATGCGCCTTGTAAAGCTTTTTCTCACTGTGTTCGATTATAGTGCTCTCAGCCTACCGTTG |
| | | CAAACAATGACTAGCGACTGAGATGACAACACGCCACACATATCGAGTGGTACCGTATTGG |
| | | GAGGGTAGTGGAGAGACCACCCGATATGGATAACACGTACAAGATGTGGTTAAAGAGCCAA |
| | | TCACAAATTGAGCGGCGATCGTGTCGACAATTTTTCATTGTGTAAGCATGCATGTATACTA |
| | | GAAATAGAGTAATACTTAGCATATACGATTAACTCTTGGTGAGATGAGATTCTAGCTTTAA |
| | | AAGAGGGGATACCGATAGAGTAATACATGTTCTTTTGAGCAAATGGGTTGTTCGCCCTGAT |
| | | CCATGATAACGACTATTTCATAGCTCTAATTTAGATGCTTGACCCAGTGTAAAGATCCGTT |
| | | TTAACTAACTTAGATGATAATGAGAAATAAAGTAATTGACTACTTAGTACACTTTAAATCC |
| | | TCCAGTCGATGTGTATTGTCGCTATATCGCAACCCGATGTTCACATACAGGGTCCTGACTT |
| | | TGGGTATACCTTAGTACGTAACAATCTCACTCACAATCAATCCAAGCGCGGTTACTATGTT |
| | | ACGACGGGGAAGCAATACACAGCTAGGCGTGCAGTACTGCTCTTAGCTCTCCGAAATCTGA |
| | | TCTAGATGCCCAAATAATTTTGTTTCCAAAGCTAGCGAGGTTTTACGACCAGTCATGACAG |
| | | ATTCTGCAGTTGAAGCATGTCACAGGTAAGCAAAAGCGTGGAACGGATGGAGCGAGTAATC |
| | | AATAGAACTTACTTTACGAGCGGTGTTACAAAATTGGGTATAATGCACTAGCCGACATCGA |
| | | TGGTGTAGTGAATTGGACTGGCACCCTCAAGGCCTCGCCCAACTCAGTCTCGCTAGTTTGC |
| | | TACCTGCATCCTATGAAGCTGTTTTTAAAAATATCGATTTCTAGCGGTAGTTAAACTATTA |
| | | GGAAGGGCTAAAACAAAGTTAATTATACTTATGTGAACTTACAATTTATATATTAGAAAGT |
| | | GAGTAAGCATATCTGAACAAGCATCATCGTAATGAGGTCGGTTCGAAGTATAAACTTAAGT |
| | | TAACGACATCTTCCAATACCATCGAAGTCTACTAAGTAAGTTAGGTGCTTAATGATCATTC |
| | | ATAGTGTAGCAAGTCCCCGCAACTAGATAAAGTCAACGACTTAGGAGTTTAGATAGAATTG |
| | | TGTACCACTAGCTCGCTACAATTGGTTTGTCTAGACTTAATCCCTTACCTGTTGAGACCGA |
| | | CTCTATTTCGGTAAAAATCGGCAAAATACGGTAACATTGTCTGCAGTCTGAACACAGACTA |
| | | GCTTATATACATGGATCAACCATCAGGTGTGACTATGTTTTATTATATGAACTGTTACCAT |
| | | GGCGCCTACGACAATAGTATATTTCCATTTCGGTTACCAGTTTTTGTCTACTTTATCCATT |
| | | AAGTGATATATATACATGTGTCCAACGTTATATGGACAGCGTTGTGCA |
| 294 | 41.90% | TAAAAGAACGGACATGGCGCACAAAATGACTATGAGGCGGTTACTTCTGATGATCACACCC |
| | | TAGTTCTTACTCAGGCTATTGTACACCCTGCCCTCTCAATATACCCGGAAATATGCATTTA |
| | | TACGGCAATCGATCTTGAATCCCAGTTCGAGTCTTTACAAATTCCATCGTTTACTACGCAA |
| | | CGTCATGCTAAATAACACCTTCCCATATATGTAGCGTGGGCGGGACTATTAGAGTCACTTT |
| | | GTGCTAAAGAGCCGGTAAGTATAATAGTTTACTCCGGAAGGTGTCAATATGTTTAGCGACT |
| | | GTATTTTGGTACTTTATCCCTAAACTTAGCTAATTTACACATATAGCAGCTGGAGGAGCAA |
| | | GGTATCATTTAATCTTGCTTAAGACCCTAGTTTGTACCCCTGTCGCACACTAAACCCAAAA |
| | | TTGCGACATTGAGCCACTTAGGCCACATTCGTTAATCTGGTAGTTACAGCACAATGGCTAT |
| | | AATATACAGATACGTCTAGAAAAAAGTTATTTAATGCATAGCTTGCATAATCGATTCTTTA |
| | | AAACAGGGTGGGGAGCTACGTATCTAGGATTTTATTCTACGTCATGATAACGAATCTTCCT |
| | | GAACGTACTAGATGGCGACTATCGGAGAATGATTTAGAACGCCGGGTGTGTCTTGATGATA |
| | | TAACAATAAGTACCACGAAAAGAATGTAAATAACTTGATATCGACTGTCACAATTTGTTTG |
| | | TATCATTGTTCGTATCATTATGCTCCTGCTCGTGTCGCAATTCCCCTTTCACCTTTTGGTT |
| | | CTTTATACACAATCATATTATAGACTTATACGGAATATTGGTTGTAACTTAGAGTAATACC |
| | | GATTGAACCCACATGTCGCTGACTGCGACGCTACGGCATCTTAAGCCGATATATCGTCGTG |
| | | ACGTAACTAGGAGTCCGTAAGCGAAGAGTAGCATAGCGATGATCGTTTCAGACTCGGAGTA |
| | | TTAGAGTTACCATGCTAGCCACATAGAACGGCCTTCCGTAACCGGTGGCACTCGTTCGCAG |
| | | TGGGAAGCCCAAGTTAGAATAAATTGCTAAATCTGATTCTCCCGTCTGGACTTCGATCTTC |
| | | GAGCTAGAGTGCCACTACGGGCACTAACACATTCAACGAGTTTCGTCGGGTGGCTCGACTA |
| | | TCGGCACGAGTGTTGCTCTACGAGAATACCTGCCTTCCTTACTGCGATTTCTCTTTACGCT |
| | | CTTCCACTGGTGCCAAGTGGCTGTATATTACTGGTCGAGTAGGGCTCGCTGATTGTCGTGA |
| | | TTCAAAAACGCAACTCTAAAATCCATACCTTTGTTGAATACCTTTATTCTCGTTATCATAG |
| | | AGGTGTTCGGGCCCTCACTATCGATGGCAGATATAGCTTCTCCGCTCGTACTTTCATATAG |
| | | ATGTTCCCCAACAGCTTTAAAGTTAGAATGATCCACTTTCAGGGCATCCAGTAACTCGAGC |
| | | AATTATGTATGTAACCGATCTTTCGATGATAGGGGATAGTACACCTTAACCCTTGTCCCCG |
| | | GTGAATTGCGGCGACACCATGCGGTAGGCGTATGTACGGTGTGCCCTTAATTAACATCGCT |
| | | ACTGTACTACACGGTTAGGTCGTTTGAAAAGGCAGCCATGAATGTTAAGATCTTATTTTAA |
| | | AATTGATCATTTACATTTAGCTGCTTTGGGGGTAAATCTACTGATCCAGGTATTAATCTCT |
| | | TTTGTATAATGTACCAATTGTAGTAGGTTCTCTATGTTCTTAAGTTTCATTGTCGATAATA |
| | | AACTAATCGGCAAAGGAAGAAAACTCAATAACTTGTATTGTACCAAAAAAGCGGGGGCTAT |
| | | AGTTAGATCGGTGACTCACTTTCTTCGATATAAGGGAAACCCACCGTATAACGACGGTGAT |
| | | CTTAAGCCTTCTCCCAGGTTAACGTATAGCCTACAAATGAATGCATTCAAAATGTCGTAAG |
| | | CCTTTTACCTGGAAAGCACAAACGATAGCGCATTTCCTTAAAGTACCT |
| 295 | 38.90% | ACTTGCACAGAAATGACAAAGACGTCGATTCACGATAAGGCATTCCAATAAGTATAACATA |
| | | ATCGTGTTTCGGGGCGCACAAAATAGATACCCAAAAGAGTGTCCTTTCCACTCGACAGTAG |
| | | AGCTCATAGTTCCGTGAGATTCTTGCCTCGTAACTAGTAGACTGTCTATCGCAAGAATATC |
| | | ACACCCAATATTTAACAACGCTCTGACGTAGTAGTGGCTACTTGTGCGAATAATCTAGTTT |
| | | CTCATATTTGCGATTCAACTTACGGCTAAACGGCCTCATAGTTTTTCCCTATTTTGAACAT |
| | | AAGTCGCTGTTAAGCAGAGTGATACTTCCCTTATTTAAGTGTAAGATGTTAAACACTAAGC |
| | | TAGAACACAGTAAGCCCCCGTATCTTAGACGTAATAGCCCTGTTAGATTAAAGGATTGCGA |
| | | TCGACATACCAACAGATGACATTAAAGCAAGTATAGCTTCAATTCCCGCCACGGTAAACAC |
| | | CTATCACGATACAAAGGATAGACTTACCGAGTACCGTAGTTAGTAACCTCTAAGCTAGTAA |
| | | ATCAAAGTTTTCGCTAGTTATTCATAAGAACAAAATTACAAAATGCGTATTTACAACTCAT |
| | | TTACAGTGATGAGACCGATTCTAATCCAATCGGTGTTAGTTTTGCTTATCTGAAAATACTG |
| | | TTAGAAATGACGTGGCTGTTAATCAATGTATAACGTGCATGCGCTGAATATCAATCATCAG |
| | | TATCGAGGAGTTGGCATACGCGGGGGCTGTTGTTAAAAATTGATCCGAATCATCTGGTTTA |
| | | CTCCACTAATGGATTAAGCCTCCTCAAGGCAGCTGATGTGAAACCCAAAGATGTCAATTTG |
| | | ATTTCGGTAATTAATTGAAATCCCTGTCCTGAGCAGACTATAAACAGATAACCGTATGGAA |
| | | ATCTGATTCCTTAGACGTTTTCAAATCTATTCAAGTAAATTTTTACGGGAATCTTAAACGA |
| | | TATCGTTCCGTGAAGTAATTCAAAAAACGGTCTTGATCTTATAATTCACGTTTGATACTAA |
| | | TTTAGTCCTCCGCTCCCTAATGATTTTTTACGAAATGGTCCAGTTTATTGTTTTTAAAACT |
| | | CTTTGGAAAATTCGTGTATGAGGATGATTAATTGTTCGATCAACGTTTGTATACTTAGATC |
| | | TCAAGCAAGAACTGTCAGCGACCTGTCGTTAGGTAGTTTGTTGCCTGCCACCTCGCGACCT |
| | | TAGGAAAGGAAGGTAATCTATTCCTTAATACGTACTATGTACAAGAGATGCAAGAAAAGGG |
| | | CAACATGAGAACGGTTAGTCTCTTTGACCCTCTTACTGGTTAGTGAATATTTTTACCAGCT |
| | | GCTACGATGCAGGATATCTGGCCCTTTGACTGTTCCATGGACACGAGCCCGAAGGATATTT |
| | | ATTTAATCGAGAGCTGTATTTAGTATCTTCATAGGACTTGAAATCGGATACCGCTGTAATT |
| | | GTGGAACCTCATGAGACCTCCTAACAAAACAAGTATCGACCTGCCCTATCTCCGACATTTA |
| | | CTCAACTCTACCCCCAGGTTGACAATTTAGGATGGTGTCTATGGGAAATATGATTCGTAAC |
| | | GTGCTGCCTCAAGAATAGGTTATGAAAATATATATATAAAATTCTATGATAGTTCCTTCGT |
| | | CTCACTCAATACTAAGTCGTTAAGCCAACTAGCTCGGGCGGGCTATTAGTTGCCATATGAG |
| | | GATCCATGAATCAAACAAATAATGCAATTCTGCTAAAAAGTGTGTATATAGAGCGTACACA |
| | | CAAGAAACAAAACTGACCGATCCGACTTAACCATTTCAATATAATGCTGCACCCTTGTCCT |
| | | CAATAGCTTGCAGGGGGCAATTACGTTTGGAGTCTGGTTGTGGTAATACTCGACTGTCCTC |
| | | GGCGATATAGAATAATTATAGAGTGTATTATAGCACAAATTATTAATAGATTCCATAGCCT |
| | | GGCGTTACATGAATATTCTCAGTTAAAGCATTTGAACGATCAAGTGGT |
| 296 | 40.60% | AGGAACAATGTTAATATCAAGTCGGGTCCAAAAAGATGTGTAAAGTTTGCGAACCGTTGCG |
| | | ATCTGTTTCTGTATCGTCTTACACTGTCAGGGCACTAGGACTCACTACGACTCATATGTAC |
| | | ATTGTTTAGCTCACTCCGAGACGCTTAGTGAATCGTTAATAGGTTGATTTGTTATTGAAGC |
| | | TGTCTGACTTATTATCTTCTTAAACGACTTTTTACGTATTGGGAGTCATAGGCGTTTTACA |
| | | GATATCCGCGTCAGTCCACGACGTGGTGCTCTATCGGATAGGTACAATCAACAAGAATGAT |
| | | TATTGCTCATCTTAATTTACTATGTGCGCCGTTTCLCCCCAAATTCGCTCAAGCTCAGACC |
| | | ATTGAGGGCGGAATAGGATTGAGGGGTAGTGAGGCGCTGCTGTATTAGGCAACCCCGGTGG |
| | | TTCATTTGAAAAAACAATCGCGGAAACAACTCTAGGCCTAAGGGGAACAATCGCTTTGACT |
| | | ATGAGCTTCTATACCTTTGAATATACACTTTGCGTGGAGCTTGGCGCGACTCCTTTTGAGG |
| | | TAATGCGATCCTACCCATTTTGGGTTCCCTCTTAATTATATTATCGGCTTTTGTCACCATG |
| | | ATCTCATAATACTGATAAGTTACCCCTGATGTTACGACCCCGCAGCCGTTAGATATTTTAT |
| | | TTAGGAGGACCTACCCAAGGCCTATGATCCTTTCTCTATATCACGAGGATTACAGACAAGA |
| | | GATGTGTAATCCGCCCAAGTTACTCTACTCAAGGTTGCGCATATTAGGGGAGGGCGTTTGA |
| | | CAGTTGCAGTATGCCATCTTGGAAGGCAACAATAAACGGTACACAACTTTACAAATATTCC |
| | | ATAATTGTTTCTACTTTTCATTCATTCATTATGTATCCCTCTATACTTATAAAACATGTAC |
| | | GACATGTCCTGTAGAGCGGGACCTGTTCCCGCTCATGACAGACGAGTTATTTGTCTCCGAC |
| | | GTATCATCCATCTTTAAATATTGAATAGGAGCAGCATCAAGTGTGGATAAGTGCAAGCACT |
| | | ATTAAATCCGCGTGAACTTTCATATGACATGAGAATCGGACTGTCTGTTATCGTAAATAAA |
| | | CCCGAGATAATGTTAAAACTATTCTAATGACTTCATGAAGCAGGATCATCTAAAGTTATCA |
| | | TAAACCACTTACTTAACCACTCATATTCCACAAGTTACGGTTCTTTAGAATATTAAGGTGT |
| | | AATGACCCATCGAGCCTTATAGCTCGAATCAAGATTAAAAGAATATTCTAAATGACCATAC |
| | | CGGTTACATGTGTGGGCGGAGTCAAAAGTTTTTCTGACTATTAGGTGCACAAAGGTGTTCA |
| | | GAACTTAACCAAACTCTTAGCACATTTGATTAGCTAGTCAGATTAAGGTCTCCACTTTCTT |
| | | TTCTGTGGTAGTTCGGTAAATTGATGGGCATTAACAAACTTAAGGTTGATTACAATGGGGG |
| | | GTTATCGGATGGTTATTGTAATTGACCCGTCCATAGATTTGCTTAAAAATCGCATTTTGAA |
| | | TACATATCCTAACTTCCAAGCATTACACAGCGCTGCACTATAGAGCTAGGATGACTGTACA |
| | | ACCTCGGATTATAGCTTCTACGTAAGGCGTGGCCGTGGCTGGTATAATAGTGGGGTGGAGG |
| | | GAGAATTGACAAAAAAAGTTTATCATTTAAATATTAGTAATGGGGTTGTCGTTCTAGGACC |
| | | GTATTTCGCGTACTAAGTCACATACCCTTATATATTTTCCACAGCAAGTCTATCATTGCAA |
| | | GCTGTTAACTTCATTCCGGCGGCTGCTGAACCAGTATCAGTTGGTCCACAGAAGCTAAAGT |
| | | TAGCAAAGTAATACACGCCAACCTACTTATATATGTATATCGTATAGCTTAATTGAGATGT |
| | | CGTAGCCATTACATGCTGAGCCTTATTTTTGACCGAGACCAGGTACAC |
| 297 | 39.40% | TTGGACGTCGAAATTATTTTTGATATACGTGTAATGATAGACTAAAGGCAAAAAGAAGGAG |
| | | TATAAGTCTAAGTTCGAAGAGGCGGATTTGGTTATACGTCCTGCACCTCTTGCCAGACATT |
| | | CTTTTAATTCTTGTGACCTGGACTTGAACTTCCTTTTTGCGACCATTTGTGGGTTTAGTAC |
| | | GAAACCCCCATAAGCAGTTAGCATTAAACCATCAGGTTTGACTCGCCACATTCGCTATCGC |
| | | AAATGCTACTAATTCATCTTAATCTGACCCCCCCGGGAAGGAAGCCATTTAATAGATAATC |
| | | TGAGTCGTTCCAGAGATGTACTTCTCAGATAAACCGTGAAGACTATTAGGACATATGCTGA |
| | | ATAACCAGTATGTATGGCTGTTGTCGACTCTCATTCCTATAGTGGAGAGAACTGATACATA |
| | | CATATTCCCTACACGGATGTTAAAGAGTCGCAGGACCTGGTGAGGCACTGGATCAACAAGT |
| | | TGCCAAACTGAGTGCCAGTGGAGCTAATCACACCTTCGGCTCTGCGTTACATGCGTTAGTG |
| | | AAGGTCCTTGAGGTGTGCCAGCAAAGATTGTTAACATATAATCTAAGGGATTATATGGTGT |
| | | ATATGGGACTGAAAACCTAGAGGTCTGTGGGGAAAGACCGTACAGTCCCTGACCATCACAA |
| | | TAAAAAATAGCCAATATAGCGTGCCATTCTAAAATTTTAATTTTTAATCAATCGCGACTCC |
| | | TTTGGTTTCATGCTAGTTGATTCTATTTAAGAATCCAAGTGAGTTTTAATCTTAACCCTAA |
| | | TGATTTAAGGTTCCAGTAAGCAAATAAACGACTCGCCGTAAAGCGAAATTGATCGATACGT |
| | | TTCTTGCTTTATTTTTGGGTACAGCAATCCTTCGAAATGTTGGCTTCGTAATTCCCTCCAG |
| | | TAACTTAAATCAGTTAATTTGCATTGTAAGAAAACAGCAAGTGAATCATGTCGCCGCTTCA |
| | | GTAACTTACTGCAAAATGAAAGCCTAATAAATAGTTACCCATCTATCTAAGTATAAACGAC |
| | | TTTTGCTTATGTCCACCCATGCTAGGCTGTGAATCCTCTTACGTATAACGTGCTTTGCGTG |
| | | TACTTTCGAACTTTCTAAGTATCAATCGCAAATCGAAGTAACTTACCACCGCTCGTAGGAA |
| | | TTGCATGTTAAAAAGGGTTAACTCCCTTCGCTTTGTCGTTTCCCAACCTGATGAAGGAAGG |
| | | TGAAATACAACATATGGAATGATATATATCACAAATACACACGACTCTGGACCAGTGCAAA |
| | | GTAGTTATAAACTCAAAACGCCCCCGACATACATTAATTCTACTTCGAAAAATATGTTGCC |
| | | CTAACGAAATGGTTTGCCTAACAGCGGCAAAAGATATGTCGACTCGATTGTATTTAAATCG |
| | | ATTATTAAGATTGGGATGAGGGCCACGTAGCCGAAACTGCAACATACCGAAATGGGCGTTA |
| | | GAATGCATTAATTATAATTTATTGGCGCTCAGCCTTAATTAACAATCTAGGCGTGCTCATA |
| | | CTGTGTACTTTAAAGCACCATTTACATGTCATAACAGATTATTGATGTTACGTAATATTCA |
| | | TAGTATACAGTATCACCTCGATCAAATTCATATGTTTTTATTTTAAACAAGAGTACTCCTG |
| | | TGTCGTTCTGAATTACTATTAGTCAGGTGCGTTAAGCTCTGCAGAACGATACCGACTATCT |
| | | GTGCATCTACCTGATTCGAAAATGAAGGCGATTGGGACTCTCCACTAGTTCTGAGTTGTCC |
| | | TCCTCGATTTACAATAGATAACTTCAGCTGGATGTTTATCGAACGCACTAATCTTAACAAT |
| | | GGTTTAAGTAGCCGTATCAGATTCGCCATTCAAATCTTTGCTCTAGTTTCATCAGTCCGAG |
| | | TTACTCTCAAAATAACAACCTAACTCGTCTTGCCTACACTGGTTCTGGGTTTTATATTTAG |
| | | AGACATAATCACGAAACTTCATGCACTATAGAAGGCACCATGCTGTTC |
| 298 | 41.40% | TGAGCTTCGCTTTTTCCAGAGTCGCTGACTAAAGTGAAGTGTCTAGTCGTTGTCCATGCGA |
| | | TATCGGGGTCCATCAACTAGAATTCATTTACGGTACGCGTTGTCATGCCTTATATTTAGCA |
| | | ATAAGACTAACGGAAGCTCCTCTGGAGGGAAAGTAAGAACGTCCCCCCGGGAACATACCTA |
| | | AAATAAAGGTGCATGAACCATCACGGAGTGGAGACGCAAAAGATCAATTAGTACAAATCAG |
| | | CAGGAGACATGCAAAGACCGCGCCCCTTTCTTTTTATACCATCTTAATAGCCTTTACTGAT |
| | | CGTGTATGTTTTCATCGTGCACCTAATTATGGAAATTCTATGAAGCTTTTGCTCCTAATCG |
| | | TTTAGTAATGCTCTCGGATGCCACGTTATCTTACTGAGAAGCCCGTGACCAAAGCATGGTG |
| | | ACAATAGAACCAATATATATGAAAATACCGGGTTCGTCTGAAGACTGTGTAGTAACAAAGG |
| | | TATTCTTGTGAATTCACGTTTTTAATCTCATCTACTATCGGATATGACAACAAACTCTGAT |
| | | TAGGGTAATATAAAATTTACCGTTCGGCCTAATTAAAGGACAACCGGTATGTAAAACAGCA |
| | | ACATCACCTAGCACGAAATTTACCTATGAGTGTGGAATTCGTTAGCGCTGTCGACGTGCAT |
| | | AACCTACGGGTTGTTGCATACGGGTCAGTGGGATAATGTTGACTCGGTCCTTAGTAAAGAC |
| | | TAGCTCTTCTTATTCTTGCGCTTGTAACTGACAAGTCGAGTTCACGTGGGCGCAGTAAAGT |
| | | CGGGAAGACGGTAATCGCAAAAGTTCGGTAAAACTAACAGTTTTTAACGAGTCCGTAAGTT |
| | | CAAGGGCCTAAATAGCTGGAGGATTTTAACGTCTAAACATTCGGGACACAGTGTATGACCC |
| | | GCATAAAAGGTTCAAACAAATAATACTTAGAGCCGTCGTTCGGATCTTATATGTTTGAATG |
| | | AACCCTTAATCACCCTATAACATGAAGCTACGACACATTAATCAGATCAAAACCTACTTAG |
| | | AGCTCGTCCGATACTACAACTTGAAATCTTCCACCAAAACTAAAGGGTCCATTATGTCAAA |
| | | ATACCATTTCTATTTATATTTTAACCATCAATTCGCCTATACCCCTAATCAGCATTAATCT |
| | | CGCTTAAAGATGGTAGAGTTAAATACAACGCAGAGCTTTTATACTACCAGTGATGGATCAC |
| | | AGGATTGCGTTTCAAAAGGTGATAGCAATTACCAATGACCTTTGACAGTAATGTTACATCC |
| | | TAACCGGATTATTTGGAATACCCTCTATTTGCTTTCTGTTTAGCCGACGCCTGTAATTGTC |
| | | TACCTGCGTGCGTTGTGATGCCGGTCCGCTCGATTTAAGCACTCCGATATCTCATGTAGGT |
| | | GTGGACTTTGGACAAGGGGAAATAACTCTCAATGACAATCGTACTGCTTATGTTAGGCAAT |
| | | GCTGGCATATGCTACTCTGAGGCTTACTAAGTTAGTCTTGTCCGTGATCTCAGAACAGTTA |
| | | CTATTTAGTTGCTTGCGAGTATATTTCGGTAGAGACGTATCTTCTACTAAACACGGTTAAA |
| | | TATTTTTTGGTTATCTCTCGCCCGGTCTAGTAGTGCCATAACGTTTACGAGGTCATATAAC |
| | | TGTCATACATTGCAAGGCGCTTTATCTCTATTGTGAACTAGTAATTATAGCCATGATACAA |
| | | TTTTTGGACGGAACTTGTTTTATCTAAATCGAAAGAACCTACATTGCCTCGGCATAGACCT |
| | | CGCAAGCAGCTAGTTCACTAGCTGCTTCATGATGGTCCAAGCTTGTGAAAGATTCACATAA |
| | | AATCAACCTCCGTGGGAGTCTCCGATGGACGAAGCTGTGTGACTGGATATTATCTCATGAT |
| | | TGCGTCACCCTTAACATGTGTGAGGTAGAGCTAACTATAGAAATACCAGTCGAGTTAGCGA |
| | | CATAATGCGAATTGATCCGCCTGTCAATTCCTCCTTATACGCGCCGTT |
| 299 | 40.00% | ATTGTCCATTCTTGTATTTGTATCACTCCCTAATGAACCAAACTCTCTAAGCCCATTCTTG |
| | | TAGTATTTAACACACATGACAACGGTCCAATTTTCATGTATAGTCGGAGTAACGCGATATA |
| | | CTGAATCTTCTGACTTATCAGACATATAAGATGTAAAAACAGCGGATCAAAAGTGTTCTCT |
| | | GCTGGGTGTAAAATGACAATTAAGCGTGGTATTATCTCTGTTAATAACACAGGGATTTATA |
| | | TGTAAGGATCGCGCCCTCATACATTCATTAATTCTCACTCAGACTTCCCTCCTTCGGGCTA |
| | | CGTTAGATTGAAATGAAAATAACATGTTGTAATCATTAAATAGTACATACTGAGTTTTTTA |
| | | AGTCGAATACTACAAAAAATATCATACTTTTTTTACCAGTTCAGTATTGGAGTCGACACAT |
| | | GATCTAACATAACAGAAGACATAGCGATGGGGATTATCGACCTTTTTATGGGTAGTAACAG |
| | | GTGGTTGCCGGATGCACTAGCATGATCAGGTCTCCTACTCACACAGTCCTTCTGACTGTTA |
| | | GGTTGTCTTTGCTTATAAAAATACTCGGATTATTGCGCCACAATTATTTGATCAACGAGCT |
| | | TCTTGGAGAGAATAAAAATATTACACTTCGGATAGATAATACAGGTTAGGTTCTCCTATGA |
| | | ATTTGAAGATCCCATGTTCGTTACCGTCCAAGAGCCACGGCTTGCTTGCTCGAAATTAAAG |
| | | TGGGCATTCGCGCGGGATGGGAAGTACCCTCAGTCTTGACAATTCCCATCGTCAATATTAG |
| | | TACGGTGGATTCGCCATCACCAGGAAACGTATTGCTGATGATGATTTCAATACTGAAGTCG |
| | | TACACTTCTCACCCGGAAACGTTAAAAGGACGATAATGACTTTATTGAGATCATCGAGGTA |
| | | CGAGCCCATGCCTTAGGTCGCTTCGTAGGGGTCCTCCTTAAAGGAGACTGTTTCTTACATG |
| | | ATTTGTTACTTCGTTGAAAATAAATCATGGATCGACGTCACCAATTACTGGGGTACCTGAG |
| | | TATATAGCGTAGAACGTGAAAGTGATTACACCTGTATAGGAAATGATGAGCTCGGGGAACC |
| | | ATAATGAATTATAGTGTAAAGATAAAAAACTTGCCCCGTGCCACGAGAAGGAATGTAGCAG |
| | | ACAATCATGGGGACATTGTAACTTACCCAGACTTTAATTTCGTTTTCACTATACCACTCAA |
| | | TTATGATGTGACATTCTGGAATTGATAGCGTATGTTGCAGCCTTCTAAACTCAACACTGAG |
| | | CTCCTTAAGGGTTATTATGGTTATATTTGAGACTATAATATAATCCGAGTTCGGTCGTAGT |
| | | GAGTAATCTTTGGAGGGTTTAGGGGGGCAGAATTCACTATAAGCAGCAGAGATTTTCTTAG |
| | | AAAGAGCCGGGTCCCGTTCCAATAAGCCCTACCGGACGTTTATAATCATTGGTGCATCAGT |
| | | GAGGCCTTCTGTTCATCTTCTATTCTGCTGTACCCTTCTTGCACCAACGCGTTGGATCCTT |
| | | GTATCGAGTCACTGCCAGGTTTGTGGATTTTTTGCAGCCCACCCTACGTTATATCTTAACA |
| | | ATCGGATAATTAAACCAAGCTATCGAATGCTATGAGCTACCACAGATTATCATCGATTGTT |
| | | TTCCCTATCATTACGATCCCTGACGGACTACTTAGTATGTCCTTTTCTTAATATTCCTTAA |
| | | GAACTGGAGTACAGGCTGATTACACAACCAGTAGGATTAGGATTAAATAGAGAAATGTATC |
| | | CGGAAAAGCGGAGTTACTGTTTGGGTCTTTAACCGCGAATCGCGGTTTTTTTTCTAATATG |
| | | CAGTGATCCTTTATTTGGTTACTGTACATCTGCTGAACACGCTATGTGGATCTCCCACAGT |
| | | TGCAAGTGCAAAATATTAATAAATTAATCACTATACAGTACAGCTAGATTTCATACTAAAT |
| | | GCTGATTTTTGACCGCACCCTCGAGAGTAATTCAATGACGGCCATGTA |
| 300 | 38.90% | AATCAGAATGAGCAGATGTAAAACATATTTATGTAAGCAGGTTATCCCGTATGGCACTCGT |
| | | TGCTCTAAGTAGATGTTTTTGTCTCGGGTAACTTATGTCCCCATCCTCAGAGTGTATTTAC |
| | | TTTTATTTAACCCGACGGTGAGAACATACAACGGGTCAACAAGACAATACGACCATTATAC |
| | | TGCTAAACTCTCTTCCTCAGGTGCTATATGAGTTACGACACAATTTTTGATGTTAAAGTCG |
| | | ACCCTAGCTGCTAACTGAACTTCTGGGACTTAAAACTACCAGAAAGGATGAAGAATTAGTT |
| | | TGGTCAATAACTATATACGAAACGCCCTGAAGGAAGTCGTATTAAATTTGGAGTGCATAAG |
| | | ACATGGTGAGCGAAAACTAACACCTACCTCTTAGATACAGATTAGTTTTAGTTATCTTCTG |
| | | GTCTATCGTTGATCATTCTAAGTTTATTCAGCACTAGAGACTTTTGGAATACGACTGCCAA |
| | | AGCTAGTATAGGATTATCTAAAGATCATTATTATTAACGGATAATGCGAAATTTGCTAGAT |
| | | CGTATATACTATTAATGCAGCAACTTAACTAAAGATATATTTACAGTGGGGCTTATGCAAC |
| | | CGGTGAGCCCTCGGTTCTTTATGATTCGTCAAGTAAAGTTGCACAACGTTCACGATTTAAT |
| | | CTTATTCTTTGATCTTGGGCTGATGTATCCTCATTATTTATGATAGAAAATTGATTGGTGC |
| | | ATTTGATTCGCCCGATACTAGACCCACAGCTGTTGTTCGATCCCGTATACAATGAGAGCAT |
| | | GTTCAGATCAACAGTAGGTGTAACATCTTATGTTCCGAGCCTTCTAGTAACCAACGAACAC |
| | | CTGGCAAATGAATTTGCCATCTTTCCGCTGTACGAATAGGGGTAATGTGCCCTTGATTTAA |
| | | AATGTTATCGATAGGGGAACTACAGATACTGAGAACTCCTGAAACGACGTTAACAAACCTC |
| | | CTGCAAAACTTGCACTCTTTGAACGAGGTTGCCTAGTTTCCAGAAGTAGGTTCTTGTCACT |
| | | TGAATTTCGATGGAATTCTCCTTATCTATCCAGTGACGAGGAAGAAGAAATGGGTTTTTAC |
| | | AAGGACTAAGTGTTTAGACAGAAAAACTAATCTTTCAGTAAAGGTGAGAAGTGATTTTGCA |
| | | GAGGGAGATTGTGTTACGAGGATAGTACTGACGTTTATATGAGAAATAGTTATCGATAATG |
| | | TGCGTGTCTTTACCAAGGGACTGACCAACTGATGTGGAAATTTAACTCTTCATGATCACAT |
| | | AATTTCAATACGTTAACAGTTAGAAGCGGTGATCTTTACAAAGTAGACAATGAGTTATTGT |
| | | CCCATAGCAATGCCTAATGTCGAGCGTGCTTCAAACAATTGAATGGCGTTATTTTTTGATC |
| | | CTTAGGAAACAAAAACCAGCAACGTAACTTATTCTTGTATCTTCATGTAATCACATTACCG |
| | | GTATAGAGATGGTTTTACATATACGCACGTTACTTTGAGATAGCGAAGCATACGAATATAC |
| | | ACGATACAATGTCAGAAGGATAAAATCACTATGGCCTCACTCGGTGCATTTGATTTCAAAG |
| | | GCTTAATGTAGCTCTGTTCGCACTCGTGGATATAGTTGGAGCCAGATAGACTAGGAAGATG |
| | | TTTGTTTAGATAGTATCCTCGTTCGTGCATAATATCCTTGAGATAGTATAGGTCGAATCTC |
| | | CACAGCAGCAAGATTCTCCGTGAGCATTGCCACTCTTTCAGTAGTAAGCCTAAGTAATTCA |
| | | TTAAGCGTAATTAGAGACTTATTTTCCATATCTGCGCGTCGAGTTTCTTCTGCAGCCCTAG |
| | | TTAGGAGACATACGGGACGCTTGCGTTTTTATCGTAGATTCACTTAGTACAGGGAAGATAA |
| | | ACATGAGAGGAAATCCGACACCTAACAATACTTTCAAACTGAGGGGCTGGATTGTACTTAC |
| | | CTTCACATCATCGAAGTCAATTCTTCACCTTCACAAGCTCTTTCTTCG |
| 301 | 43.30% | ATTTACACCCATGCCGAACATAAATAAACAAACACAAAAGGATGAGAGGAATAATGGGTTA |
| | | ACTAAGGGGAGTCGAATCGTATTGATACTTATGAATGGCTATGTTACACTCAGGTTGTACT |
| | | GGATTTCGTTTGCGCTACAGCTTAGACCTTTCGCTAAAGATACACGCCGCAGTGTCTGAAA |
| | | CAGACGCACATTTAAACCGCTGGGCTGTTAACGCTCATTCTCGCTGAACTAGTCTGTCATT |
| | | TATCAGTGAGATCAGCTTATCTCCAATCCTCATAAGACCGTCGACAGGAACCCTCAATTCC |
| | | ACTCGTAACAGTCCCACGCTGGGTTGCGTAGTCTGTTGTAAGAATTCATTCATGGTTGAAA |
| | | TGGGGCTGATGACTATGAGGCGGCATCTATTGGTATGGTTTAGTAGACGATCAGAGGAAGT |
| | | CTGTATAGTCAGGGCTCAATATGTATCCACGTAGTAATGTTGCCTGCTACCGACACGATTT |
| | | AGACAACGTCAGCGTTATTACGAACACGACCTCGGTTCCACGTGTCATCGTCTAGATGGTC |
| | | CCTTTGTTCGTAGGCCTCCAAGACCTCAGTAATATCTAATTCGAGCTTCAAGTTTGCTAGA |
| | | CGTTGACTTGACGTAGCAGATAAATCGCACTGTAATGGAATGATACCTGAATCCCGTTAAC |
| | | TTCCAGCATGGCACATACGATTTTTAAATTACGCTTTAGATAAAGAAGCAGTGCGGTCTAA |
| | | TCGAAAGTGCACAAGCATATCAAAACTCAGGTCTGGTTTGTACGATTATTTGGAGCAGATT |
| | | TTCAAGATAGTTATGCCAATCTCTCCATAACCATATACAGTGACGGGGACCCTCTATGATA |
| | | CGTCATCTCCGGGACCTACTTTGACGCTGGAGTCTTACAGATGGTGGGACCATTTGTGCTT |
| | | AAGCTACTTTTAGTGCGGTAGGAGCCCTCCACAATATGATTCAAACCTAAAGAAGCTAGGA |
| | | GCCCTCTCGACCCTGGTACTTGGCATTGGCTTAAATTTCACGTATACGCCATAGCAGATTA |
| | | GTTTAATCTCCGATTTTCAAAATACTAGATAGGGAGAGTTCTATACCACATTAACTCGCCC |
| | | CGATGGGAGAACGCACAAGAGTTAGTTTTCGACGCCGCGTAAAACAATTCAACATGGCCCT |
| | | CGAGTCTGCTACTGTAGTGCATGAAAGCTTTCCTAGTTGGGCTAGTAGCCCAAGATTCTGG |
| | | AAAAATTCAAGTTAGTCGACAGATGTTTCCGCCTTACGAGTAATTTAAAGAGGTTACCCCG |
| | | AGACCGCAAAGAGTTTAGTGCATCTTATGTGCATTGTGTTGTTCGTCAGGGGGCTTTGCAC |
| | | CTAAACGGTCTTACGTACAAGCTCAGTTCGTGGATACATGAAAGTCTTGGAGTCAAGACCT |
| | | ACAAATCGACGCGATTCTAAGTCTAATGTATCCTTACTTCGGGCGTATTGTGATAGTATCA |
| | | TAACGGTTAAGACAGTTTAGGATAAACCGCAGAGACAAAAAATCTCGTTCGTGTAACTGAG |
| | | TATATAGTGTACACTTGTGCCCGCAAATGCATATTATTGATCGAGTAATTTAACGTGTGCC |
| | | TCCTTGGTAGAGGGTTTCCCTAACATACTCCTTTTCCTGATTACCTCAGTCTCCTGCTTCA |
| | | ACCGGTCTCCATAAGTGAGAGGTTGTGTGTACCGCACTTTAGAAGAGTAGAGGTTTGGCAA |
| | | ATTTTGGGAGCATTAGACTAGTCGAATTTCATACTTCTTAGTCGTCTGGGAGAACGTAAGA |
| | | CCTGATTAAACGCATGATACACGTAGTCATTCAGTTCTTCAGTTAAGAGGTTGCATCAAAT |
| | | AGCACTAGCTTAAATGTAAATCGTCTTAAGTCCAACTATTATGCGGCACTTGATCACCATT |
| | | TCACTCACCTCATCACTACGCTTGATAGTATGATCTCATCGTGATGGTACCCAGTTGAGAT |
| | | CAGCGAGGATCTCCTCATAAATTTACACATTGTTAAAAGGTCCCGCGC |
| 302 | 41.50% | TAGATCTGCTTTGTGAATGCCGAATTTCAGATTGACTGTCCGCGCGCTAGCTCATTATGAC |
| | | CCGGCAGTTGAAATCGTATAGGGTTGGACCCAACTACTAACGGAACTCAACCACTCGCCCT |
| | | GTACGAGATCACAGGGAACGTCGGCTAAGGAGGTTATGGTGGCCTTACCTTAGCACTATAT |
| | | AAAGTGCGTTCGAAACCTCAGTGATTCCCCGATAGTATGATTTTTAAGTTCTAAGATTAAA |
| | | TTTGATACATCAGTTGGTCCTAGAGTTAGTGCTACTAAGCTTAAATCAACCAAAATTTTAC |
| | | CCGTTCTATTCAGAAGGAAACTATAGTGGTAGCAAGTGTGACAGTAGGTATAGACTTAAAT |
| | | AGTTACGGCGAAATAGAAAGATTACGACGTTCAGCCTTGTGTATCGAATTTGTGACTTTAG |
| | | AGGCAGACAGAGTAATGGACCTATCATCTAGGTCCTGTCAGAGTATCATGTGCATGATTCG |
| | | ACAGAAATCTCAATAATAACCCAAATCGGGCTCTCTTGCATTGAATAATTCATCATCAACA |
| | | TGAGGTAATAGCAAAATGCCTTTACTTCAGTTGATTAGGGTGATGGCCGATCACCTATGTA |
| | | TTTGAACATATATTGTATATCCGGTCGGAATATGGCATCCTTAGCCGTCGTGCGCCGGCTT |
| | | TCGGAATTTGATCTGTCTCTGTTTAGACGCGTAACCTCAATTCGCCGCAAACTAGATCACT |
| | | ATTCTAATAATCTCACTAGGAATCTATTCGACATGCGATCTTTGATTATAGGATTCAGAAT |
| | | CTAAGAAATTGCTACGATGGGGTGTCATAGCGATGTCTATTTGAGTTTCTATAGTGAATTG |
| | | GCCATTTGTTTTGGCATCATAGATCGCTGACACAATCATTGTGTCTTTCATCGATCTGGAG |
| | | TACAGTTAGAAGAGAAGCGAGGGCTGGTAACATGCTTATAGATTCTTATACTTACTACCTT |
| | | AGGGTACACTAACAATATTTGACATTATAGGTCGACCAAAAAGATTTCTCTATCAGGTTTA |
| | | GAGACAAAGTCGTCGACATATTTCTGTTTGAACTCTTGAGGATGCACGAAAGTGTCTATCG |
| | | GGGTATCAGTGAGAAGGCGTGGCAAGCATTCTCTAGGTGAATTCCACCCTTTTTAGTCCTC |
| | | GTTAGTACCCCGTAGACCGCGGAACATCGAGAAGTTATTCGTAAACGTGTCTATCTGTTCT |
| | | ATGTTAGGAGTAGGTCATTGAACAAATTGAGCTTTCAAATAGATTCTAGAATGTAGCGCGT |
| | | AAGTATGTCCCGATAGCGGTTTTCAGTGTATTAGTTGCATCTAATGTAATTGAGATGAAGA |
| | | AAACCTTGGTCGAAGAGACATGCCTAAAGAAGAAGGCTAAGTGAAGGCCTTTATATCACGT |
| | | GGTTCATAGOCCATTATATAAAAATTTATATTGGAGATGTCCCATTGGTATTGATAGATGG |
| | | TTGGTAGCTGTCAGCAGTGCGCCCTAGGTAAACCAGAAGACTCCTTAACAGATCGGTATAA |
| | | TTATTCGAGGTTTCCGGCTCTAGCATTCAGACATGGAAGGTTCTTTCTAAGCGGATATATT |
| | | GCTCGAAGCCCGTGAACCTTTAGAATCAACCTTTATTATCTCTAACCATCTTTTTTACGTT |
| | | TCACCTTTAACTTACGCGAATCGATTCACGACTGCCGAAGTACAAACGATGACTCAGTGTT |
| | | GGTTTTCGCTACAACATTGAGCTCAGCTCTATAGCGCGGACTACAAGTTCTGCGTAGATTT |
| | | TGCCAAAAAAAGTTGCGGGTAGCCTTATTCATTTAACGTATGACTGGGAGGCGCTCAAATC |
| | | TCTCACTGCACCTATTCGCAGACGCAAATTATGGCGTCGACCCCAAACTTTCAGGTAAATA |
| | | GCTCACAAGATTGACCATTGGCAAGTTTGAACTAGTGTCGTAACGTCCTGAACAAATGTTT |
| | | TTCTAGCCGCTCCTGCTAACCTTATGGACATTTTCCTCTTCACCCCTG |
| 303 | 39.40% | AAACTACAGAAGAACCCAAAGGCTACTCACTCCCTTTGCTGTGTTCAGCTCGCTGGCTCGT |
| | | CAAGATAACGGACTCATGTCTGTGGGCAAAGCAATTTATTACAGCTATACCTTTGTGGAAA |
| | | AGTCTCCTTGTAAAATTGTTAGCAATATTGTTTCGAGTTATATCGAATTTAAGGTTTATTG |
| | | TTATTCGTGACCATAAGGAGCTAACATGATGCGGTTTAATGCGTATGGAAAAGCGATAGTG |
| | | TTTTTAGTGAGGGAATGTAGAAGACCTCGTTTCAACCCTTACCATACCCGAGGGTGTCTTA |
| | | ATCTGTTATTAAATAAAGAGCAGCAAAATAAAAAAAAAATGCAGTGTCTATCAAATTCCCA |
| | | AATTTGGCTACGTCGTTCACTACCAATTTTCAAAATAATAAGAAGAAGTATATGGATCCAG |
| | | TCTGATTGTCTTTCCGATCAGCAATATAAAGCACCAACGTCTTATAAGAGCTAAATAGTGA |
| | | TGATTCCATGCAGTATAATTCAATTCCCCTAAAGCTACTGTCGATAAACTTCATATAACAT |
| | | ATGTACTTGGACCGTTTGGTTTGGACTTGACAGGCTTTAAGCAGTCTGCATCATGAGCCTC |
| | | CTTCTAGATGTGCAAGCATTCCCCAGAGGCGGTTCGCTTCAGCGTGGTAAGGAATGATCTC |
| | | TGGGTCGGAGGTAGTGCAGAATGACCACTTATCCTATCTAGTGGTTTACTTTATCTAAAAC |
| | | AACAGGGGACTAGATCTTATTATACGGCCAAAACTGAAATGAAGATCATCTCATGAATATT |
| | | CTCTTAACATGAGAAATTTCCGTTGTCAATTTTTAAATGGATTAATGTCATAAAATCTGGG |
| | | ATATGGCGAGCTTAACACAATGCCCCTAGTTTACGTTAAGAAACATTTGATACATCAACAA |
| | | AACGTAGGATCCGCCCCGGTTTTTTGGAATCCACTTCTAGAAGCAGGAGCGGGTCGCTGTA |
| | | TTTAAGTCATAAAGGACGTCGTTTTACGAACAAGACCGTGTATGAATCTGGACTGTTACAA |
| | | CGGCCCATCCCCACCACTAGTTATACTAGTCACCGAATAATCTGAACTATTTTACTAGAAA |
| | | GTCTAGAAATTCATCCTTTGACATAAATGGATTGGAATTAAAAAAAGAATTTCAAATATAA |
| | | TCATATAAAAGTGGATGCACCAGAGCTCATGCGACGTCATTCTACGAGCGATTTATAGCTT |
| | | ATACCAATAAACCCCGCGTGTATTAACGGTCCAGTCAAAAATACTATGATACCGAACAAGG |
| | | TTTATCGACTTGTCCCGTTGAAATCCTAGATGAAGTTTATAACCAAATGGCGCCCCTTTAG |
| | | TGACGCTGTAAACGCAGATTTATCAAACAGGAAACATTTCTGATTAACCAGAAGTATGCGT |
| | | AGTGAAGGTATATCGCGCAGTAACATTCAGGTGCTTCGGGGATTCAAAAACGTGTTGCTGG |
| | | TATAGCTCGCCTGTTTTATCGAATGTAGTCTCAAAATCTAGCCGAGTTTATCAACTGGTCG |
| | | ACGCTGGAAGTCTGCACTTGAACATCGTTCACATGTAAGCCAGAGATAATGGCCTCAGCAT |
| | | CGTCTTATTGCTAATCTCACGCTGCTTTGTCGCGACGTACTCTCTGCATTACCAAATGGGA |
| | | TTAGTTTAATTTCGTTCTCTGGGTGACCTTGTGCACGCTATGTGGGTTTGTATTAGTTGAT |
| | | TAAAGAGTCCCTTTGAAGATGGCTTCACTCACCACATGACTACACTTCCTATCGAGGTAAG |
| | | GAAACGTTTTCTTGTGCAAACACCCCAGACTTACGAAGTTTAAAGTTTTGTATAATATTAA |
| | | GAATTTATCTAACACTGAGACACCATACACAGCTTCCGTACCCTATTGGTCCACAATATAA |
| | | GACGTTAGATATTGCCAATAAATGCTTCATTCGGTTTTTTGTTAGACAATTGGAAAATCTT |
| | | ATACATAACATATAAACGTTTCGCATCCCTGGTTCCTTCCGATAGGTC |
| 304 | 40.50% | TCGTTTTATCACGTTTTAACATTGAATCTTTAGTGCAACCAAGAGCCACTTCTCCTGGGTT |
| | | ATAATCATCATCTATTTAGCATACCAACGCGTTTGGCTGCCTCGGTTTGTATATAGTCGTA |
| | | AAAGCCTCCGGTTTATGAGGTGATGGAAATTAGTTGGATACTTGAATAGATAATATCCCAT |
| | | GCGGTATTCACCCACTGAATCACATCGCCTGATGATCCTTGCTGTTTGCGGGAGAGCTCTT |
| | | CTAATGATTTTTGCAAATGCTGTGCATCCCTAATAGTCTTTTACAGGGCAAAGTACAGGGA |
| | | TTGACAGCCCCCGAATGTCTACAGCCGACAAACCGAAAGTCTTCTACCCCGAGGTAGCTGA |
| | | AGGTGCATAGACGTAGACATGTTGACTAATCTCATCTTGTCTACTATCTTGTACACAAAAT |
| | | CAAAATTAGAATTATATGGAAGGCATGGGATGAGTGATCGTTAATTAGACAGGGGCGTCTT |
| | | TGGCAATGCATTCTCTTATGATAAAAGGTTGACCAGATTACTGCTCATGACTTAGTGTCCA |
| | | CCGGCCCAACAATTAATAATTAAGAGACTCAACCGACATACGTTAATACCCAATAATGCCC |
| | | CAATACCCAGACTTTTACAGGGTTATTCGTGAACATGAGTCCCTCGACATCTTCCCAGATT |
| | | TTAATCCCCATATTACTAGTTTGTAACAGATTGGTTATGGGACTGATTAGAACAGGGAATT |
| | | TCAGCTGGAAATCACTACTAACTTATTGCTAGTTTGCCGATCTAAGAAGAGTCTTTGCTAA |
| | | TTGATTTTAAAGAGATATTCTGAACACGTCAATATCCAAATTTTATCCGCACCATTCTGAC |
| | | GTAATGACGCCTAGAGAACGAGTTGGTGGCAGTCTATCGCTTCTGTTTATTTTAACCTTCA |
| | | AAATATGATAAGGCCCCAGTTATAAACTATTTTTTACGGCAACTTCGGATTAAGTGTTCTA |
| | | TACGCCAAAACTATTGATTTACTTTACATTTCATCCCGAGAAGCTCCGTCTTATCAAGTAC |
| | | GAGATGATCCCCTATTAGAAAAACCACGGCTAGTATCAACGACATGCGTTACACACACGCC |
| | | TCAGTGGGGGCCGTCACACATAGTTCAAATATTGATACTGCTCGTCTCGATATGTGTTCAA |
| | | TGTCGGCAATCAAGCAGTGTCGGAACTGAACCCGCACTACGGGCTCGTAAACGACCCAAAA |
| | | TCCCCTAATCAATCATTGTAGTAATGGTAGCAACTTGTATGTCCTGTCAACGCAACACCCT |
| | | CCTGGTGAATTATTCTATTAGAACTACTAAAAAATAAACCCGAGGTCCAGCTCTATCGTAG |
| | | ACGACACGAAAACGTATCAAGGTACAGTTCGATAGCCGTACTTATTATGGTGACTAGCGCC |
| | | ATATACAAGGTCATTAGGGACCTTGTTAGCGGTGTGTTCACTTCATCGTCAGCGACTCGTT |
| | | CGACTGTCATTTCAATGAAATCTTTAATGAGTTTAATAGAGTAGGAAGGGACAGTAAGATA |
| | | TTTTATGAATAATGTCGTACGTAGGATTTTTTTCAAATGATGACTATCACAGTACGGCATA |
| | | CGGAAAATTCAGTAGGGAATTAGATCAAGTGTAAAATTACTGGTATACTAGCGTATACCTA |
| | | GTACGATGATAATTAACAATCACCCCCAGCATGATGTGAGAATAGTAAAGTATCCATATTT |
| | | AGAACTAAAAAGCTCGGAAGCTGAAATCCCAAACCGCTTGAACAGCTCTCGAATAATACCG |
| | | GTGTTTATCATCGGAAGGACAGCGCCTCAGGATTTTCGGCAAATCATAGCTCTTATCTTCG |
| | | ATCTAAGCGTTTGATGAATATTAGAATCGGACTGAGATATAAAGAATAGTGATATATGTCG |
| | | GAAAACGACGATGTCATTTTAGACTATGATCTTAAGACGGAGAAAGCTACCATCATAACAC |
| | | CGACTTGTCCTGCCATTGTATTACTGGCTTTCCATCGTGAGGGATAGC |
| 305 | 42.10% | ATTATGATCCCAGGCTTCGTTGAGTCTAATAGCTATCCGACTAATGAACTTCTCAGGCATG |
| | | TCTCGACTCCGATCCTGGTGGCCTTAAATTTCTTAGGTGCACGGAATTGTGTGTACCTGGT |
| | | ATGTAGAGACTATAACGACTCACTTCTTGCCAATTAGGATTCAAAACTCCCTACTTGAGCA |
| | | ACGTGTTCCCCCGCATTATCCATATCACAACAGTTGAATTTTTCTTACGTCTTCTCCTCAA |
| | | ACCGGAGGGAAGTGTGAATGTACTGTTGTCCGGCCATGCCTGAGGTATTTTGATTCTAGTT |
| | | AGTAATTACATTAGGAACTCACTTCGTCAACTCAAACACGTTGACAAATGTGCAGTTGGGT |
| | | AATACATGCCGTGCAAAGCATGTATGACCGTGGTCTACTAGATGGCTTCGCGATTTACTGT |
| | | TTATAAAGATATGGCTACGACTTAGCTCGTGAGATCGAGACAAAATCAAGATCTTATCGTC |
| | | TTCCACAAAAAGTACCCTCAATCGGATATTCGGACCGTAAAAAAGAGCATGGCGCTTGATT |
| | | ATCGTAGCTAGCGCCCAAGGAACAATTGTATTATTCAGATTAAACCCCGGATTGGACCTAT |
| | | TTTCATCCTAGTAGAAACGGTGACGACGCGACTTCCGAAAACTCCAGGAACAGTGCGGTCT |
| | | ACCCAGGTTGTAGTAGATGCCCCTTTTCTCAGGGCAACCAGGGCATCATACGTTAACTTAA |
| | | TCGGTTTTAACCGCGAAGTTCGATACGGACTGATTTAATAATAAACGCGAACAACCTAGTA |
| | | ATATCATAAATTGCGGCGTGTACTTCAGAAATGGTAACTAAATGTCAGACTTCTTGAAAAG |
| | | GAACAAGCGCGCTTTCTCAAGTTTGTTGAGTCTCATCATAATGGGGGAACTCCGTACATGG |
| | | TCCGATGGACTCGATATCCGAAGGCGATAATAATTATCCCCGTGTTCTACGCTATTTACGA |
| | | ACTATTAATAATGATCGGTCATGTCGGTGGTTTATTCCATTCCTTTATCTCCGATAAGTAC |
| | | GTTACCATGGGATTACGCAACAGCTAGATTTTCAAATGATCGGGTCGAATCCGGCCTAAAC |
| | | GAAACGTCGCTAGCGATTGAGAACGGATGTACAGATCTCTCGAATACATGAGATGCGCGTA |
| | | ATCATAGTGTACGATAGAACCTCATGTTATCAACAGGTGCTATCTTAGTAAAATACATAGT |
| | | CATATTCTTTACACGCGTAAAGATTCTTTGAGCCAGCGAACATGGAAATGGGCGTTGGTGT |
| | | GTTTCTCCCCGGCTTTCGTAATAGTCGCCACCATCCGCTTGGGTGCTGATTCGATCAGTTC |
| | | TAACCAAGGAGCCTGACAGTCTTCGATTTTTGTGTATTCCTGTAGAATATGGCACCATAAT |
| | | TCAGCGGGAAAAAATTGTCAACTCAGCAGTGTCTATTAAGAGATTACTCTCGCTTTTGGAC |
| | | TGGTACAGCCTTTACCTAGTAATATAGACGGACAAAAATTTTGTGAGTCAGACGGCATATC |
| | | CTGAAAACAAATACAAGTGTAGTCTACGTTTTAGAATAGACTGAGTGGCGTCGGTAGAAGT |
| | | TACTGCTCGAGTTATTGTAAAATTCTTGCCAAGAACGAAGTTACTCCATATGGAAAAGATG |
| | | ACTCAATCGAGTCTTACTAGATTATTTCCGAAGTCTTAAACGTTTAGACCTAACTTAGTCG |
| | | AAAGTTGAGCTCCAGAAGTCATCTCTCCCAGTTTATCAATAGTGGGTGGAACAAATTCATC |
| | | GGCTGTTGACCTTATTGCATCCACCTCGTTGGAGTTATCTTGCCATGTATCCTCAAGTGTT |
| | | CCGACCTGGAAGTATGTAGAAACCCCTTTGAAATATCTATCACAAAGCAATATCTTATATT |
| | | ATCTTCGTAGTTTTTAGAATTATATCTATTTAAGGGCACAAAGTCTAG |
| 306 | 41.70% | TTAACAATAAATGATTAGGTTGTGCTTGCCTCCTAATTTTGTTTAAAAAGTTGTTCTTCTG |
| | | CTGACTAGTTTGATTCTACTCATTTCTGTAGTACCGGTTCGGCGTACTTTTTTTAGAGGAA |
| | | AATACTAATGTGCGGAGGAGGGCTTAAGAAAACTGCAGATCACTGGATGAGCAGGAAAACC |
| | | GAAGGACGTGCACGAAAATCGGACTTGCTGTTGTGACTATACGCAGGCTAGAATCAATACC |
| | | GTCGGTGCTCGTGCCTCAGCCGTATCAGATATGATTCTTGAGCGATGTTATCGTTGGATCA |
| | | AATAGTTCTTTTCGTGGAAAGGTATGGTTAGATATCCGGGGCCTCTTAATATTGGTTTCGA |
| | | CTAGATCTGACAGAGTCGGGTCAAAGCTAACGCTGTCGCTAATGATGACAGTGTCAATCTG |
| | | GTTAAGTATACTCTGGAGTTATTAGTCGATCTCTCTCAGTGTTTCTTAAGGTGTTCTCAGC |
| | | TGGCCGGGTTGTGCGCTTGTGAGGGAGCGATAGCAGTTTGTGCTCGGTCTACGCAGTAGAT |
| | | CGTTCACAACTTAGTCAGACCAATTTATATTCCTATGCCTAAGAAATAGTAGATCATCTAA |
| | | ATGTAGTTGCCGATCAACTCAAAAATCATGAGCAGTGATAAACGCTAGTACGGAGCTAGCA |
| | | TATGCGCCTGCCGATAGATTGCATAGAACCACAGAATCTCTAAATTTCTGGCACTGACTTT |
| | | ACCTTACTTGTCTACTGATCATTTAGTTCTAAGGCGGGTCCCAGCATATACTGAGTAAAGG |
| | | AAATTGCAACGGTCCAACAAAGAATCAATAAGTAAATAGAACTCATCAATCTCCATGGTTT |
| | | TTTACCCTGTGGTATGAGAGCTTCGAGACAGTACAAATACATTCTACGAGTGCATTTATTA |
| | | AACACACGGACCCTATACAAATTAATAGCATCACTAGCTCGAAACCTATTACAGCCTGAAC |
| | | GTTTCGAACGCACTTCGGTATACAGTGTACTCGCGCGCGTGTTGAACCGAAGGTGCTAGCC |
| | | GAATTAGTTGGATTCGTATATATGTGGGATCCCGATTTCCAAGTCCTTGCTGGTTTAACAC |
| | | ACGGATATTAGTTGCTATTATTAGCGTGTTTGAAAACCATGTCAGAGTTAACGACCGGCTA |
| | | AAAAGCCGACTTATAAAAAGCCGAGTGGTTTGGCAACCTTCTACTGGTCTTGGTATTAACT |
| | | TCTGAATAAATAGAAACATGAAAAGAGTGAACTGCTAGACTGCACCTGTGGAATGATCCAT |
| | | AACAGTTAAATTACTCCGCCGAGTCCATTTTGCTGACGGTGGATTATCCTAACTGAAGAGC |
| | | GTACAGCGATTCTGTCCAACCGTTGAAATCAGTAATTTTCTATACCTACTATCGTTTGACC |
| | | AAACTCAGGGAAGCATACCTAAATATCATCAAGGCGAGAAACTTTTAGACCCATAGTTGTA |
| | | TTATAGTCTAATTTCAATGCACATTCTGTTCAGGCACAGACTGATATTGAAAGAGGCCCGC |
| | | GACTTTGAAGGTGGGCTAAATTTATGCAATAATGGCACACGAATCAACACAGTCTAGAACT |
| | | TACCAAACCAAGCCTAGATTCACCTATCTATTTTTGATCCGACTGTATAACGTATTGTAAT |
| | | ACCTCAAGACATAAGACACTCATAACAATTTAACTTTCTCTTATTAGGAGGCTCCTCTATG |
| | | GGATTCGTCGTCGAGTTAAATGATTTGAGGTTTTATGTGGACTCCGAGGACGCCCGGTAAG |
| | | AATTTCTAGGACTTAGGATACAATGCAACTCAGTGGAGTATGTTCCCCCGTGTGATCTATA |
| | | TGATAGCTGAGTACGACAATAGGCATGCGATTCAGACTATCCGCTTTTAATTACCAATGAA |
| | | TGTCACGACGGAGAACGTTATGAAAGGTTTTCTCTAGCACGCCCTATCGCTCTTATATGCG |
| | | AAATACATTCCTGCTTGTGAATGGCCGGGATTGCTTACACATTAGCCT |
| 307 | 38.40% | ATATGAAGCACCTAAGAGCTCTATCCCCCCTTAAATGTCAAGATTGGCTAATATACCACCC |
| | | CATACACATGATTAACCCGGTTACCTTCGACAGGTTTGGATCTTTAAATACAATTAGTTGA |
| | | TCTTCGCTCTGGCAGAGCTCGGGTTCGTTCGTAGTGTATAAAATATCTCTACTTGCAATTA |
| | | TCGTTTTACCCCTGCAAGAGCGTCTATTGGTCTTGCTGTTTTCTTACAGTTGTATGCTCGC |
| | | CATGTATAGGCAGGTAAACAGACTTTGACAAGGGTGGGCGAGTCGCGTAGAACCTTTCCAT |
| | | GAAGGCATTTATTTTTGATTATCTCTGATACCTGGGTGTGTATAATTGGATGCAACGTCGC |
| | | TTGCTAAGACATTCGAGCTCGAAATTCTAGGATTTTGTCTATACCCTTTAGAATCTTCACT |
| | | TCTATAAATGACTAAAAACATGGGAAATGACAAATTAGCAAGCGGCGCTTTTTTGAATCAA |
| | | TCACTAGATATATTTCTAAAACTTAGCAATGCTTTCATGAAAACCACTAATTTTAATTAGA |
| | | TATTTGTAAATAACCCGCATCAAACGCAAGTTGATGTCGCATCATATATATCTCCATAGTC |
| | | ATTTCTATTCAACTGGCATGTTCGGTTAATCAAACAAACCTGACAACATTATTGGTCTCAT |
| | | CAAAATTTGCTCTATTGGCATCCAGAAGATTGAATTTTGAGTGACCAGTAATATTACCCTC |
| | | TGGGACTACTTGTATCTTTTGTAAAAGACGTATAATTGTAGGGAAAATTTGAAGTTGTAAA |
| | | CTAGAACAATGAAATAAATCACAAGCCTCTTAAATTTCCGAGTGTGTTTAATAGCTGTCCG |
| | | AAGAATAAATATCCAGGGAGGATCTGATCTCTAAAAAGGAAACTTTCCTAGGTGCAATTCA |
| | | TGGGACAATAGTCTTTACCATCATTTGGATCGGAATCTTTAAAGATTTAACGTAAAACTGT |
| | | AGATGGGTGAAGCAACCACTGGTGTCAGGATTGTTGTAATAACCTACAATACGAAAACACA |
| | | TGGAAATATTTTTTTCACGAGCTATACACGTAGTTATACGTATGAAAACAAACAGGACTCA |
| | | AATAATCTATAGAGGAATTTATAGGTTCTTCGTGAACGTTTCGAGAGCATAGACATGATTA |
| | | CAGGCTGCAGATGATTGCTCTAGGGACACTGGATACGTCTGTCTCAGTATATTAAGAGGCA |
| | | TTAACTTATAGAGCTGGTTTGAGTTCCTCATGAGAGAGAATATATATTTGCACAATGATAC |
| | | TCAAAAACTTACCGCTCTGCACAATCCGCACATCGCGATCATACGCGCCGTTAAAGTTATC |
| | | ATCCAATATACTCATAAATGGTGTAACCTAGCTCCTACCACAAACTGAGTACCGGGATCGC |
| | | TATCCACATCGCTGAAAGAATGGGAAAAGAAAGGTTTCCTTCGAGTCACGCACTGAGTAGA |
| | | TCTACAATACTTATGCTCTAGAACGCGTGATATTTCTATGTAAAGTAAAGCATGCTACTAA |
| | | GGTACATCTAATTTTACGAAACCGTATACTACTACTCGCCATTGGTATACTTTAGACTTTG |
| | | TAAGTAAAAAACGAGTAGGGCCTCAAGGACATAGTCACTGCTTATACAGCGAAACGAAGCT |
| | | GCTAACAAAGCTCAGACCGGTATTGCTGTTAGTATATTCTTGTTAGAAGCGTACATCGGTT |
| | | GGGCCGTATGGTCCGATTACCTTAAGAATAGTTGACTAGGATCGTCTCTAAGGTCGTACTT |
| | | ACCCACCTAGCAGCTGATATCTTCGATGCCTATATCTGTATAGGTAGAGATTCATTCTCAG |
| | | CGCATTGCCGCGGTAGATCCTATGTAGATTATTTAGCATAGTTAATTA |
| 308 | 39.10% | GAACCTTGGGTCCTTATCCTGAAATAAAAAGAAAGTGCACGTCTCCGTAATATATGGATGT |
| | | CTCAGTGATATCCACGATTACATCAAGCTGAGTTATTTTTAATGATAGTTGACTGTATTGC |
| | | CTAAAACGTATCTGTAGTAATGAATACATAAAGGTACTGGTGATTGAGAAGTTCTCATTAA |
| | | ACGTTAAAATCCGCATCATCTGTAAAAGGTGGGTAATTGCACTATAGAGGGTAGACCACGC |
| | | CTGTAGCCCGCTTAGAACAATTCTTGTACTATCATTTTTAAGTCCTTCAATGTCTATCATA |
| | | AGTATTGGACATTGCACGAGAAAACACGGGACAAAATGCTCGTCGTTTGAGACTATGGATC |
| | | GCTATTCGGGTCGAGCAATCTGAAACAGATATTGTCATGTTTGGAAGGTGAGCCCATTAGT |
| | | AGTAAGCGCTTTATACCACTATTCAGGAGTTATAATTTAAGGAGTGTAACAGTATGATGTC |
| | | TACCGGTACACGGGAGATTGTAATACAGTAGTAGCTCCTTATGGCTTGGGAATAAATTACA |
| | | AACTGAACGCTTTCTTTAGAGCTCTAGTGTCCTGATTTATGGGTAAGGCGTATTATCTGCA |
| | | AGTCTCAGTTCGGGATAGGTATTCCGTCATCTAATATTACCTCTAGGGTGTATACTACCAT |
| | | CCTTTGCAGACTATAAATACTATCTATCGTCGGCACTGATAGATGGAGGATTCCTTGCAAG |
| | | ACCTGATATCTCCGTCTCCATGTCTAGTTTATAGATTTGCCTTACAAGTTCATTTATGCAT |
| | | GTGTAATAGAATGATTTATATGAACCGTCATAGTTCCATTTTAGCATCCGAGCGTGTGTCC |
| | | TCTCTCGTAATTAGGCGTACGTCGAATCATTTTGCTTTCACTGTAAATAGGCAAAGCAAAA |
| | | TGTAGCAAAGGAAGGAATGAAATGATCATTCTCATGCTACATGTGTCCTTATACATAAAAA |
| | | TATATATACTTGATTAATTGCACATGAATCACTTACATTCGATTATCATAATACATCCCCC |
| | | ACTCGGATTGCTCCACGACCAGATGGTTAAAAAGTTGAATCTGTGCTTTGATTTTTAAGTG |
| | | AGCACTCACGTAGTATGAAACCGCTAGCTCAGGTTTTTTTTGGGGATCGTTCAGTATTCAC |
| | | GAAAGAAGAATGCGGCGGGGTGGTTCCACACCATATCAACTAGTGTTTATAGTTGCTTATA |
| | | TAACGGCAACCGGCTAGTAAATGGTAACTTAACAGTAAAATGTCTAGGATTAGTAAACATA |
| | | TATTATGGAGGCGTTAAGGCTGTACGCCTTGATAGTACACACCTTTTTACAATCACAATCC |
| | | TAGGTTGATCTAAAACCGTTGACGTCAAGTCCATTATAAAATCTTAATCGCCTGATTTCCC |
| | | TGTCCTAAAATGAAGAGATTAAAGAAGTGAAATATATCCCTAAGCCAGAAGTGGGAGAATA |
| | | CCATTTGGATATATGCGAGCTTCTGCCAAATCTTAGAGATTTCTGGACTTTTCAATTATCC |
| | | AATATGAGGCTTGAGGATTACCAACTCTGGACTACATGACAGTTCCACAGAAACTATTTAG |
| | | TTAGACGCAGAGCCAATTAGAACCTCGACAATTAGGTAAAGTAAAGTTTACAATACTGTTA |
| | | AGTCGCGTAAAAAAGGTTGATTCAACTATGACGGGTATAGAGGAGGAAATAGAGGCTCTCG |
| | | TTAGCTGTGTCGTTGGACATAGTAACTTTTTACAAAGAATGTTAGAGCTGTTGAATATTTA |
| | | CGCTTATACAAAGTATCTGCTGTATCACGACGGATTTTATCCATGCAGGGCAGTAATCCAT |
| | | CAGGCTTTTGGAGAGGACAGCCTTGGGAAGGATATCGTCACGAGGCGTTTCGCACTCAGAC |
| | | ACCCGAAAAAATTACGAGGAAATGATAATCGTAACGTGGCGCCTAGCGCTGGATAATTACC |
| | | ATAATTTAACAGAGGCCACAACAGGTTTTCACCCTTCAATGAGTGTAA |
| 309 | 41.00% | GATTCTGTACAATTGTTTCAAAATATAGCTTAACACATTTGATGGAATAATAAGGGTTCCA |
| | | ACTAGATATAGTTAGTTAGGAGTTACGGGAGTGGTGCTCGGGTACACCGAAGCGTTTATGT |
| | | CTAAGCTCTCTTCTGAGGGGGCTCAGACAGCTGGTACAATAATTCATCCGAGCCGCGGTGA |
| | | ATGCGGCATCAGGCCCCTTCTATACTTATAAAAGAGCATATCTAATTTATTGGCATATTCC |
| | | TGCAGGCTACATAAAGTCACTCGGTCGAGGCATCCCTATTCGGGCTAAATTTCAACACGTC |
| | | TGGTTTGAATAGCGACTGTTTTTTACAGATGGCTTGGATAACCAATCAACCTTCAAGAAGC |
| | | ACAGTTCTTATGTTAGGAACCGTATGCAACCGTAGACTCCTATTTTCACTTGCGTGAGCAT |
| | | TCAACGAAATTGGGAAGACAGATGGACTTACATTAACGTATCGGACTACGATCGTAATATC |
| | | CGTGATGTGAGTATTATAGTATACAAGAGTGAGGAGATGGAAATCATGACGGTTATCCCAC |
| | | GTAGCAGCACACGCAGATGCAGACCAGACAGATACGAATAAACTTTTTTGTACGGTTGCCC |
| | | GGTAAACTAGCCTGGGATCCCGCGAACAAATGTTAGAATAAAAACGCGAGAGACTTGCTTT |
| | | AGTAGCTTTTCATCAGGATTCCTTGCAAAAAGTTAACACAAAGTAAGCGTGTTGTTAGTAA |
| | | TGTAATGTTTGTGAGGTAACACTGTGGGTTAAGTAGTACTAATGATCTTTCTTTGCTGTTT |
| | | GACTTTCAAAATGCGTGGAGTTCAGTGGTGGCAAAGATTGTTTAAGTCTTACGTATTGGTA |
| | | GTACTCGTTAAGCTTGAAAGTTTCGATTATCTCTTTTTATTCCGATCTGAAATGAGCTTGT |
| | | TCTATCCGAAGCTGAGGTAGTCCACTTAGACCGATCTATCGCTAACGAGAATAATACTTAT |
| | | TATTTAAATCCTTTCTCATGCCAATAGAGGAGACTGTCATGGTAACCGGTATGCTTGTGTT |
| | | CATATTAATTCTAAGATTTGCTACAGGATTAAGTCTAGTTCAAGTCCTATTCCAAATACCA |
| | | CAATCTCTAAGGCCTCACACGCCTTAACAGAAAGGGGATTATACGCGTCGGTTGTTCGTTA |
| | | TGCCTTATAGTACTCAACCCATAAATAGATCGCACATAAGAGTATGAATCGGTTGATGAAA |
| | | AAGTACATAACTCACTACAGTGCCGGATGAGAGATTCCCGTGAATTAACTAGTGGCTACAA |
| | | AACGTAACGTGCGAAGAGCAAAGGTGGCCGCGATATTACCTTTACTTTCGGTGCCTTAGTA |
| | | AAAGAGGATAATGGCAAAATGAACGTCCTGGGCAATCAGACCAGAGGGAATATGCTTAGCT |
| | | ATTGGCTTTGTAATTGTTGTAGTTTTTAATGGTTCTAAATATCAACAAATACCATCATGAT |
| | | AGTTACCGATCAGATGAGCTTGAGCCGTTGAAAAGAATGCAAATACAAAATCTTGTTCATT |
| | | AATCCGATGCAACGTGCCGGCTTGAAATTCATTTTCGAAGTAGTGCGTCCCCGCGTATAGA |
| | | CGCTACAGTAGCTCCGAAGGTCTATTGTTAGAACAACATTTTAGAAACGGGCCTAATAGGA |
| | | GTTCCTCGGGAAAAAGAGGAAGGGACAAGTTGATTGTCTATTAAGATAGATGATCCTATTA |
| | | TAGCGATGTGAATACTACGCCCAGTGACACCATGAAAATAGACTGGAAATGATGGTACGAT |
| | | TGGATGAGTAGATCATTAGCTGCCTTTACCTTCGACGACTTCGTCGTAGTGAGGGTTCTGA |
| | | CCAATGTCCATAGCAGTTGAAAGCGCGACATTACTCGAACAACGCTGTGGTCACTCTTTAA |
| | | TGATTCGTATAATGAATCTTCCTCTGGAACAGTTGGACAGAAAAGTGGCTTCTTGCTTAGG |
| | | ACCTAGCTAGACTTTGTTGCCTTTCTATGTAATACGTACGCAAATTCC |
| 310 | 41.40% | CAGTAGATGAGGATAAGCCCAAGTATCGATTCCAGGAAGCCGCCATATGGAGATATAGAGG |
| | | TATCTCTGGCTTCGCGAACTCACAAAGGAGTGTCTCGATGGACCTCCATAGGTAACAAAGA |
| | | TCAAGGCCCCTTACCAACTCATGTTCTATAAACTGACATCTATGCAATAAAGTTAACACCA |
| | | GAAGGTGGGTCAGACCACAAACCACAACCCCGCTCAATTTTAGAACAAAGTCTACTAAGAG |
| | | GTGCGAATCAAGCCGAAAACGGGAGTTTATTGTCCATATGATGCTGGATCGGATTATTGTA |
| | | TTATAATAGCCTAAGATCGTGTCTCCGATCCAAATGCGTGTACGCATCAATCCTGAGAGAT |
| | | CCGGGATGGTTGCTGGGGTTAATAACTTCTCCTTTATATCCGGATGACTGCTAATTCCTCA |
| | | AATGCAATCATTCTGGAATTATGAGGCCTATTAAACGAATTTAACAGTACCTAGTCGGTAG |
| | | AAACAATTCTACCCCGCATCCTTAAGTCTACTTTCAGAGCTACTGGCGCCTTTGACGCATA |
| | | GGTAAAACCGGCGACTAGAGGAATGTCGTATCAAGATAAGCCCTTATTTACTTATGCTAGC |
| | | CTGTGTTCGATAAATAAGATGTCTGAATTGAATTCGCGCAGAAACCAGTGCTGCCACGGTG |
| | | AAGAGTGATCGGGGCGGCTATCAACTACGCGGTGAACTACCCCAAAACATTTAGGACATGC |
| | | GAATATATCAAAGAGAAATCAATTCCATTAGTTCGAAGATGAGCACGATCGTTACTAACTG |
| | | CAGACAAAGAAGGCACTATTGATAGAACCGATTGACAACCCGAACGTGTACCGGAGTTTGG |
| | | ATCAGATCTTGAGACTGCGCTTAAAAGCAAGAACCCATCACAAAAAGGCAATAGCATTAGG |
| | | AGGAATCGCGCACAAGTACAATAACTTTTTCCGTATTTTAATAATATTAATTGTCCTTCTC |
| | | ACCACGAGGCCGTTTCCTTCGTGGAACCAGTCGTCCTACTTTCTCTCCGTAATTTCATTTT |
| | | ATTTAGAATAAAGGTATATACGGACGACTATCGTTCGGAACAACTAATAACAGTGCTTGGA |
| | | GGTGAATAGAAGTAAGTTGAACTGAGCTAAAGTGAACAACTACAATTCGTAGCCCTGATTT |
| | | CATTGTCATTTTTTTTCTGACTCAACACCCCAAAGATCGCGCAAAGAATAAGGCCATAGCT |
| | | CAAACCCGAAAAAATCTTCTAAGGCCTGATAACTTAGTTATTATATGAACACCGGTAATCC |
| | | CTGCATGCAGCATATATGAAATAAAATGCCGTCGTTTTCATTGTTTCGTATAAGTAGGGAA |
| | | CGAGGTCCATGTGCTATTTTGCTCTTTTATGTGTGCCCAAGGGGTACTGGAATGTCGAGTA |
| | | ATACTCAGTCCTTCAATGCTCATCTTGTGACCAAATTCATTGGGGAACTCCATTGGGAAAG |
| | | GAATCTGTGAGAGTGAATCCAGACTAGGATCTACCCACATTGTAGTCTGAATTTTAGCTTC |
| | | TAGAAAGTACCGCTCAAGTTGACTATATTTTACACAATGTGGGCTGATGGCTGGTCTCCGG |
| | | TTGAGGAAGGATCAATCATACTCATCATGCATACATGAAGATATACTAGTATGATTAACAA |
| | | TAGCTTTTCAAAACAGACACTCGACTTATTGAGCACCCTATTGGCTAAGCAACTGCATCTG |
| | | CACTAGCAATGGATCTTAAGGCATCATATAACCGGTTAGGTACTTTCTTGTTAGGTAGAAC |
| | | AACACGGTTGATCAGGCCAATCGCTACTGAAGTAATGAAATCAATAAACACTGAGTCTTAT |
| | | GAAGTACTATTACAATCTCCTAGGGTCGTATCAGACCTTTGTTATGTTTTAAGGACAATGC |
| | | GGGATCTCTCATCCAAAAAGCGAAATTGATACCAGGCATTGGTAGTCAAGATTACCGAATT |
| | | ATTTTACGTAGGTCATTATATGCCTGCAATTTTGGCGCTTTACGCTCA |
| 311 | 38.90% | GTTTAATCTCCTTGACTAACAGGAGTCTCTTGCCAACGGATGTACGTAACCGTATGTTAAG |
| | | ACATTATGAAGAGTTAATATTACATGCAACCATTCGATTTGCCATAAATGTACCGAACGCC |
| | | GTTATATTTACTTACTGGATGAAAGATTCAAGAATCAATATAAGTTAAAATCTTAAAAAGA |
| | | TCAATCATACGTATAAAGTCTATTTGCTATTAGAGACGACTGTCTGATTTGATGATGCAGC |
| | | GCGTTGTTATAAACCTCATAAATAAGAGGCGGTGGCTTTCTTACTATTAGCACAAGTCTCA |
| | | CTGAGTAGTAGAATAACTCTTACTCTATATGTTTCATCAGGTACGACCCCACGTGGCAAAA |
| | | TTACATTTTGCACACGAGGCACATTAAGACCGAAGAGAACATTTGGCCGAGAGGTATGTCA |
| | | AAGCCGGCTTAATGATATCGACACAACTCATAAATGGTGAAAGTTATAACCAGGTAATCTT |
| | | ATGGGATTCTGTGGAGTAAAGCCCATTGGACTTCGGAATAAATAAGCAAGCTAATCAGTTA |
| | | TAATAGCATATATGTTAATACCAAGCGTGGAATGAGCACATTTTGGCAGTTTAACACTAAG |
| | | CTTGATAAAACTCGTAGAGTAGCGATTGGACACTACAAGACGCGTGTTTCGCTAGAGACGA |
| | | ACCACCTTGTGCCAACAGATTACTCTGAAGCTCGCCTATTTGTGGAAGTAAATATTACGTA |
| | | ACGGTTATAGCATTGTTAACGATGATTTTGTCGAGTAACGGTATGAATTTATGAAAAACGT |
| | | CAAACAAGCGTGATCAGTTTCGCATGATCGAATTGAGTTTTTGCCCGCGCAGGGTTCGCGT |
| | | CAAAACACCTTAGAGTAAATACTTAAGAGGAATCGCTACGTCTATTTGTAAAAGTCCGAGT |
| | | ACCCACCTTGGAATCCCCATTTTTTTTTTTCCAGTCAGCTCAACGGTTGAATCCACGTGTC |
| | | CGAAGAAGCTCTGAGCAAACTATGGTGTCGCCGTTCTAAGCCCATTTCAAACGTTATGGAG |
| | | CGTTGTGCCTCTTTGTTGGCACTTGTTATTCACCGCGGCGAAGTAACGCGCTCGTCAAGCG |
| | | AATCATTTTATGCCTACTCGGGCTATAGTTAACGGAGTTAAAATGCTTCAAGTGTAGGTCG |
| | | ACAAAAGATCAGGAATTCGAGATAAACTCTCCATGTGAAATAGCAAGTTTACGTCCTCGTT |
| | | TTTGATTATAGACTAAGATTACGAATTCTTTAGCGCTGGCTCATTTGAATCCAAAACCGTA |
| | | GAATAAGAACCCCAGACTTATGTCCTCGAAATTATGAGGTAAGAGAACAAATAATTCACGA |
| | | GTACTGACAGTATAAGCGCTTATGTGAGACGACCACGTAACTACAATTTATAAACTTGACC |
| | | GTTATTATGTAGTATTTAGTGGCTCATAAAACCAGCTTAGCTTAGATCTGTGAGACTGACC |
| | | AGCTGACCCAGAAGACTTTTACATTGAAGTTGCAGCTATATGGAAACGTACTTTATAATTT |
| | | CTTAATGTAAGAATAAATTTGCTGTATCGCTTTGTTCGTTTGAACTCTTTTCTATGTAAAA |
| | | GGCTGACTAACCCAGGAAGAGGGGAGCATATTTTACAAATTAGTAAGCGCTCTCTCATTCA |
| | | TTTAATGATCACCTTATACCGACTTCAGCCTATGGAAGATCTTGCGCTGTTGCGTACCTAC |
| | | AGCGGGTAAACGGATGTGTTAAACACGATAGTAATAGTAAGTTTCCGTTAGGCTGTAGTTT |
| | | ATAACAGTAACATAAGTGCTAACGAGATCAACACAATTGAAGTTGCGAAAGCAAGAAAATC |
| | | TTGCTACATATATCTTAGATAAGTATGAAAACATAGATTGCGTTTTTACAAAAAGTACGAA |
| | | AACATTATATTCTCAAGCTCACGCTCCATGAACATGCCATGGATGCGAGAGCTACTTAATA |
| | | TTATCCGGTAATTATTAAAGTAACTACCGGTTGCGCACAACGGCTTAA |
| 312 | 42.00% | GACTCTTCTTCTCAGTCCACGTTTGAAAATCAGACAACTACATATTCAATGGAAGCGCTGA |
| | | GTCGGAGTGGCTTTCCGATTGACTGCAGGTGTCTGGCGATAGATTATTAAAATAACCGAGG |
| | | ACCTCATCTGTGATTACTTATGTTAACACGTCGTTACAAGCAAAATGTACAGATCGTGTGT |
| | | GGGTTAGGGGTTCACTAGAATCGGTGGGGCAAATTTGCCGCAACCGATATCGTATCTGTCG |
| | | CCATTTAGTGGGAGCTGGGCGTGCTATCAGAATTTATTTAAACGGTTTGGGGACAAAAGAG |
| | | GACCTTATACTGGTAGTATACCTTCTTTAGTCTTTGCTCCGATTGAATACACCGGAACCTA |
| | | ATTTGTAAAGAGGCCCAGATGTTGGACAGAGTGGTTATGAGTGCAGGTTTATAGTTCAAGC |
| | | ATCAGAATAGTATTAAGATAAAACTGAGGGCTTTCAGGCCTTGATTTAAATGTGAGAGTAT |
| | | TGTCAGGCCATTTGGAAATATCATAAAATCCTTTGTGCCAGATAGTTATGAAGCTGCTTAG |
| | | ATCCACTTGCCTTCATTTGAGTCTGCTGACTGCCAATTAGAGTCCTCCTCGGTACGTATGA |
| | | ATAGAAAACTTCAAATACGATTCTCCCCAATTTGCTCTGTGCAGCCTTGCCGATAGTCCTT |
| | | TATGTCATACACTAGGTGTGAGCTCCAAGGGTCTTGGTTCCAGCCCCGCAATTCAGATAAA |
| | | CATAAGCCCCAGTAGCGGAGGAGATTTTGAATACCAAACTAACTTTATAACCCGCGCATGG |
| | | CCAGTGCCATAGCGAATGCGCGGGGAGAAGTCATTTTAGAAGCCTATCAGGCGATCCCGGA |
| | | TCATTACCCTCGTATAATAAATAGCCTTAGCTGCAAGTTCGTGTCGCCGCGAACGTATTCG |
| | | GTATCAGACTCTGATGTCCTTTAATAGTGATTATGACGACTGTCATAAACTTTGTAGTAGT |
| | | GTATATTATCGATTGCGTTTTATTCATCTTGATGATGGGATACATCTGCACTTTTGAGCTA |
| | | ATCTAAGATCAAATATCTATTTTCACGATCCCGCTACTACGGCTCGAGAAAGTTACTTTAC |
| | | CGGACCGGGCTTAACACAAGACTTACGACGTCCTGGATAGAATTTTAGGGGTTTCTAAATT |
| | | GATCCGGTTTGAGAACTTCTTACTTATATTCCAGTTTCGAGGACTAGGCATTTCTTCATTA |
| | | AGACCGAGGCATGGGTTATTTTTATATTGTGATGCAAATCGGTTTGCCCCGCCGGAGAGAC |
| | | TACATGCCAGTTGGTAACGTGACAAGGCATGTGCAACGTTCTTTAGTGTCGCTACGGGATT |
| | | CTGAAGTCTACTGCTTACCTGATTATACCACGGTTCAACTTCGGTTACTAAGGATATTCGC |
| | | TATTGCACGGGATGGAAATTTATTCATGTCCCAAAAAACAAACTCGACAAAGGTGCCCACA |
| | | TGCGGCCTCATTTTACAGTGCACTTATGAGCTATTGCGAGCTCCCTCCAAATATTGGTGGG |
| | | ACAGTTAATAAAAACGATCTGATAAAAATAGTAGGTATCGAGACCTAAGATTGGAATCATC |
| | | ACATTCGCGTGTTATAAGATTGGAGATGTTCTAACTTGGATGAAAATGTTAGTTACAATAA |
| | | CCATATCCTGGTTCGAAGAGTATTGAGATGGACTTTCGAGATTATAATATGATTTCAGAAA |
| | | GGTCGCACATGACTGATCCTTTCCTCTGCAGGTGGTCCTGTCATCGGGTATGTTTTTTTCC |
| | | TCTAGATAAATGGATATTGTAAGCAAATAGTAATTCCTGCATGCTGGATACCATACATGAT |
| | | GTGACCGCCATAAGCTAACCAGCTTCTAAAAAAATACACTCCTTGCTAGTATGGTGATTAG |
| | | TTACGGTGCATGAAAATAGTAGGAACGCTGATTCTCGTTCATTTTGTGTGCGTTCCACGAC |
| | | GAATTTCTGTTCAAAGTCCTGCAGATCTTATTGAGACCTTTACAGCAC |
| 313 | 41.20% | TCGTAGGCTAATAGAAACAGAATTATCAATTCCTTATTTAATACATCACTGGACTGAGTCA |
| | | TTCTCTCAGAGCAAAAGGTAATCGCTTCATTAAGGTATTGTCTATCCTGTAAGAACACCCA |
| | | CGCCGTGGATATATCTCAACATGTAATTAGGGGGTACATGCAGTGTCGCAAAATTCAAGCG |
| | | CCAACTGGGGCATTTCTAGTTATGCTAGCTAATCTACTCTTGTAAAGGAGCTTTCGACTAA |
| | | AAACTGCCACTATAATCTGATTCAATGGTGGTAATAAGCGGTAATCTTTAACCGTGTTTTT |
| | | GCTGTCCGACTTAGTGAATTGATACGTTTATAGGGAAAAAATAGGTCGCTCAATATACCTT |
| | | AAAGATAATATCACCGGCATGCGCCTATGAGGTATCGATCCTGTGTCTATGAGGTAAAAAA |
| | | CGAGACTAAAGTTTGACTGTATTAATAATTATGAAAGGGAACCTTGTAGTCAAAAGATTAA |
| | | GAGCAAACCCGTCTTTCAATGACAAGAGATACATTGGATGCCTCGAAATTGATTATTAAGT |
| | | AACCAGAACCAATGATTATACTAAGAGCTTATTCCTTTCTCCGCAGACTCTTAAGAAACAA |
| | | GGACAACTGCCCCTGAGCAACCAGCCTGCTGATACGTCCAAACAACCCGTTATCATTAGCC |
| | | TGTATTGAGCTAAAAGCACGTTTATTACTTACATGGCAAGTATTATTTATTATGTGGCTCG |
| | | TATAGGTCGGGTATAGAAATGTTGGACATTACAAGAAAGTTCAATCATAAAGCGAATCGTT |
| | | TATGTTAGCAGACTTTATCTACAGTTAACACGAGGCTAGCGAGATGTGCTACTTTTCAAGT |
| | | GTTTGGAATGCATCCGAGGTCACTATAGGCAATTCTTTACCGCGATCAATTCGTATTTGAA |
| | | ACGCCCGGCTAGCCTCCCATAGATTCCCAGTCAAAGGAATCAAGGCTGCGCCATTCTGTGA |
| | | TTTACTCCCTCTTTGGACAACCAACGTACTAGCCTGCAGGATACGATGCCAACATTAATTT |
| | | TTATAACCGTGAGATCAACGCGGTCAAGGAAAAAGTTAGGCATAATATCGCGGACACCCTG |
| | | GCGTGAACGATTAACATCTGCGGGATATGAACATTTCTCGATTTACTTTAATGATACTTGG |
| | | CTTCATAATAAACATAATACATCCCCCTGAGGTTGATAAACGTTAGAAACTTAGGCGAGTC |
| | | CATAAGCGCTTTAAAGGATCTTTTATCACACACGCGAAACATTACCATTCGATAAAACTCT |
| | | TATCACTCATCCCGAAATGCCAGTTTCGCACATGCAAAAATAAGCCTTCGAGATTGGTCAC |
| | | GCCCGATCAGTCGTCTTTCGCTACCTAACCTATGATAAAATAGTTCTTAGGAGTCAGGCAA |
| | | TTGACTTGCCTGTGTCTCTTTGGAGGCTTCCAAGTTCGGATTTAAGGGTATATGCCTGTTG |
| | | TAGTCGGACAAATAGATAGGATAAGCGCTTTCCAGGCGGACTACACTATTAGTAACTATCA |
| | | GCGAATATAAATGTACTCGGCAGCTTAAGCGTAGACTTAGTACTCGCAGGACCTCTTGCTC |
| | | GTTCTAGCATATATCCTGGTCGTTTTTAACATTTTAAGCTCGAAAAAGTTGTCGGAAGATG |
| | | ACTCCATTAGATGGACGATTAACGAACAAAGGTCTGTGAATGACATACACATCTGATCAGT |
| | | ATTGGCCGCATTCGCAGGATAGTACATCGCGGGGCAGACGTATTAAATCAACCTCTCCACA |
| | | CCCGGGTTTCGTTTTGCCATTGTTGCCCTCGACAGCAGCGTTTCATTAATAGGAGGCTTTA |
| | | TAATACGTCCAGAAGGTGTCAGAGGCCTACGAGCTCACGAACGTATCCTCATAAACTTATT |
| | | GTGTCACCAGTCAAGTCGTATTTTATCTCCTAAAACGACTTACCCACACCTTATGGAGGCT |
| | | TAGCGATCGTGTATATATGCTTCTTATTATAGTGCACCCTGGGTTCTA |
| 314 | 41.20% | ATTGGGCATTTCGTCGGACACTAAATGAACATTAAAGGATTGATCTTAGAGTGCTATATTG |
| | | AATCACTCAGCCCAGTCCTTCGGACTTCCTTGTATTTCACTGGGCGTATACTACATTCTCA |
| | | AAATAATTTTGCGAGTCAATTAAACTAGATACCACCTATGGGGGGTTTCGTCTTGGTTTCA |
| | | AATTAGATGGTAGTAAGTTTACGTGAACACCGTTGAGACGTAGACGGCTTTTATGGGTTGT |
| | | CTGTGTTAGACTCATTGAGCTGCTCATCCGAATTATTCATTCAGTACTATTTAGCACTTGG |
| | | ACATCCCTGCTAGAGCTCTGCGAAATGCGGTATTAGGTCTGGGGTGACCTCCAGCTCAATT |
| | | AATTTACACCGGTAGTAACCAAAGGTTAGTTAAACTCACGAAAATGATACTCACTGTTTTG |
| | | TGTATCCTTAGTTATATGTCGGCGGATTCAACCTTCGGATAATAAGTAAATGGTCTCAGAT |
| | | CGTAGCTGCAAAAAATCGTAAAGCAACTGTTGTTAAGATTGGCTACTCCTAACAAATTCCG |
| | | CCTCCCTCAAGCAGGACACTTCGGAATACAATCCGGAAATATGGCGTGAACCCTCTATGAT |
| | | CGACTGATTCCAATCACGGTTCAGTCCACTCTATCTAATTAACTTATCGGGTAGATACTAG |
| | | AAACTCACTCAAACCGTATTCGTGAAATAATTATTCGGAGTCAGTAAGCAAAGCCCAGTGT |
| | | GTATTTTACACTTAATTGGCTCTCTGTCAACTTCTTGCAAATTAATCCATTACTTGATAAT |
| | | AATATATCGCGTTCAATGGCAAGAAATCCACCGCAGAATCGCAAATGGACTCCCTCTCATC |
| | | TAGGTTTAAGCAAAAATGTTGAGATTCCACCTAAAAGTGGATATAGAAGACAAAATTATTT |
| | | GTACCAACAGTAAACAGGGACGGAAGGTGCCTCTCAGGTAGTTACTGAATACCTGTTAGAC |
| | | GGGTTCTGCCCGGCTTCTATGACTTGAGATTATGTGGTTCTACAGTATATCATCCGTCTAG |
| | | GAGTGAACCTAATGAAAAATACTCTAGGTTGGTACGTATTCATTCACATAAACGGATGCGA |
| | | TGAGTTGGCGGGTTGGAAGTTCTGTTAATGTCGTAAGTACTTATAGGCTGACAAGAGGTAA |
| | | CTGTGATACGAAAGGATTCGGTCTCGACGGCCGAACTCTAAAAGGTCTCCTTTTCCGGAGA |
| | | ACACAAGACTCTTCTGCTTCTGACCGTATTTGGATAGATCCATCGGCGGTACCTTTGTTTG |
| | | TTGGATCGTAACATCTCTTTTGATCCTACTATGTGCCAACTCAGTTAGTTCGCGCTGAATT |
| | | AAGATTCAAGATCCTGTTCATATCTTTTATAAAACATGTGGATGTCTTAAAACTCATCTCT |
| | | TCAAACGCCATTGCTCGTTTCTGGAGTGTTACGGGTTCGGAGTAGAGTGGTATTGGATGTC |
| | | AATATGTGAATTTATCCACTCTGACATACACAACGAGTCCGAGAATTTTAGATCGTGCCTC |
| | | CAAACAGCGCTCAAATCTTACAAATATTAATGTAGAGCCATGGCCCCATGCAGAGATGTTA |
| | | CATTCGCATGGATCAATCTAAGTTTGTACAAAAGAAAGGCACTTCTTAATCTGAACTTCAT |
| | | ATCGTGTTTCCCTAGCGATTACTATGATTCTAGTGTAGCGTTAGTTGCTTATGCTCTTTAT |
| | | ACACTCGAGGTATCATGTACCAACAACCTAGCGAAACTGATACTGAGAGGTTGCAGATAGT |
| | | CTTCGACGATTTAGCTACTGTCATTTAACATTCCTGCCTAAAATAGCTTCCGTCCACTCAC |
| | | GTACTGGATCTCATTCTCCGCGAGCCTTATAGAGACTGGATTACGTATATTCAATAATAAT |
| | | CTACTCTAGACCACCGACCTCATCCCTTGTTTATTGATAGTGGTGTCCCTAGCTGACCAGT |
| | | CTTGTTGGGAAGAAGCATGTAACATTCCTATTAGCGCCAACAACGCGT |
| 315 | 40.70% | AGAGAACGTGTCACGTACTAAGTGCAAAAGAGGCTGGGTTTTTTTTGTTAGCTTAAAACAC |
| | | CAATAGACAGAAATCCATGGAGATTTAAATGCAATTATTAATCTTGATCGAATTGTCTTTT |
| | | AGCCGACAACCTGTTGGTCCCGACAATAAATTTAACGATTGTTTTTATCCTAAGATCAACC |
| | | GTTGACGAACAAATTAGGCGAAAGTTATATTAGTAGCCAGACGCGTTTGGAAACAGGCAAA |
| | | AACTGCTAGAATACCCGTAGAAACCTACTGGAATAAATGAACCGATACGTTACCGTCTCAG |
| | | GAACTACTTAGGTTTGATAGACAGTGGAATGCCATATGTCTTTTAGCGTAACAACCCTAAA |
| | | ACCTTATTATTGGAAATTTACCAGGTAGGATGTCATGTAACACGCCAATCCAATTCATGTC |
| | | ACAAAGTGATTAGGTATACTAGCATTTATAACTTGGGTAAGTGCATCTCATGTAAGTACCG |
| | | ATGGGCGTACCTCTTCGATGTATTAACCAGCACCCACTTCATACAAGTTCATCGGTAAGTG |
| | | GTTTACAAGAAACATCATAAATAGAAATAACACCTCTTCAGTGATAAGCGGAACCCCGTGC |
| | | CACTTGAAACAATCTCTCGCAGATGACCCTTGGAACAGGGCTGACAGTTTGAAGTGACAGG |
| | | GTGAAGTCATTCCTTTACAATTTAAGCCGGGAAATTTATCAACACTAAACGTAAAATAAAA |
| | | TTGGCGTACTGCCTGGACATTGGTCGCAATGTAATCTTCTTTGTTCTCGTAAACCAAACAA |
| | | TAATATTTTGAATCGTATTATATTGCACAGGTAAGCCACTGCAATTAAATTAGAGCCCATC |
| | | ACTTCCCGGGCTAATTGAGACTAAGTCAAATTATCCTTTCAGACTTCTTTAACCTAAACAT |
| | | GAAGAGGGTTTTGGAATTGTTAAAGACATTCCATGGGGTACTGACGTAGTACCAGCCAGAG |
| | | TTCGATTCTTACAATTCACACGTATAGGTAGAGGGTCCCACAGCTACATATCCTATCCTGA |
| | | GCCGAATTCTCGCCATTGTTAGCTTTAAATATTTCGAGCCAGACCTGTGGAATTTAGTGAG |
| | | TTGAAGACTATGGGAGCCATACCGAAGTTGCTAATAAAATTGTTTCTAATTACTCTTCGTA |
| | | CATCAGAGGCACGCCATGTGTGTGATTAATTCATCTTGTTTCCCGTACAAGCAATAGCAAT |
| | | ATTGCTCGCATCACGTCCACCAAGTAATTATTGTATAGTTACTTTGAACTATATCTCTGTA |
| | | GCATTTCGAGTGGTGCTCAGAGGCGCGGATCTTGCCTGTCGGGGATTGTGAAAGTTGGTCA |
| | | GAAAGTTACAACGGTATGGTATTTTAGAAATCGCGAACCTGATTGCGTCCTAACGCGATGT |
| | | TATTAGTATTCAACGGTTGGTCAGAGTTATATACCCCTAGAGAGGCCTATGGAGATAGACA |
| | | GTCTCGCGTATCTCATCATAACTCTTGATCAATCTAGTCAAGTAGTTCACGGGACTAGCCG |
| | | TACACAATAAGGAACCTAAGTGCAAAACCACTCTTTAGATAAGGATCCTGCGCCATGCTTT |
| | | GAGCCGCAGCATTCTCTCGATGAGTCCAGCGTGGTTTGCAACACTTAGTACATAAGATAGT |
| | | TAAATACAGAGCGGTCCTATTTTGAAAAAGAAATCCTATGGACCGCACCAGCCGGAGGTTA |
| | | CCTAAGACTTCGGACGAACATCCTTGTTTAAATGTATGACTGGATGACTGATTTTCAACAG |
| | | AGCGAGGTCCAAGAAAAACTACAAGCCACTTATTAAAGACATGAGTAAGGACGAGTTATTG |
| | | AAACTAAGACATACGTGGGATAGCTAGGTGGCATAATACAAGCAGATAACCCCGTACGATT |
| | | CAAACGATCTTAACAAGTATTTTATTACAAACGGGCCTGGTTTTAAGAGAAAAACGTGCAG |
| | | TACCCTCAATATGAGTAATAAGGGAAGTGACAGGGAGCACTCGGCGAT |
| 316 | 40.50% | AGGGCTTGCATATCCACAAAAATGAATTTATCTAGGTTCAATTACGTGTTATCCACTCCAG |
| | | CGAAAACTTGACACTAGGATTATTGTCTTTTGTCGACACGTTAATACAGCAACGTCCAAGA |
| | | GATCTCTTGCTTTGGCTTGAACTTGCAATATTCACGGGTTGTTTCCATTCTTACCTCGACT |
| | | GGCTAGCTGAATGACCTTTCACCTGGGTTACGATGTACGCGGGGCACTGTGGCATTAAACG |
| | | AAGTCATTATCTGCACCAACCCTTGATAACAAAATAAATATGGTCTGCGACACCTTGTGCT |
| | | GGGAGACAAAAATCTTCTGTAATTGGTTCTGTACGACAGGATTAGTTCCTCTTTATTTCTT |
| | | ACCATGTTTCCTCTTCCAGCATTAAGATGGTAAATTGAATGTATAGTGCGCGATACGGAGC |
| | | ACGTGTCAGTTGTCGCTCGGTCGTCGCGATTATTGCTTGGAGGATCCTAATAAAGCTAAAT |
| | | GAGTGGAGTAGTAGTATGCGTGTGTGCCGGCCGTAATATCTCATTCACGTGCATCATAGCG |
| | | CATATATTCGACACTTGTAATCCCGTCTTTCGAAGAATCTAGGTTAAATGGATACTACTTT |
| | | TTACACACGCATCCTGCCTCTCGGCGGGAAATATGTTATTAGAAACTTCTGAAGTTGTCTG |
| | | GATTAAAGTACTGATCATGGCTAAAACACTCTATTTTTGGTGTGAATATAGCTCTATTTAC |
| | | TTCTATCGAGGCCTCGTTCTAGAGGTTATTAGTGACAGTCCGTCCGTAAATTTTCCTGTAT |
| | | ACTCGTCTTCCTTATTAGGGTTGAGGTGTACTGCATGTCTTATGCTATACAATCAGCGTAC |
| | | GATCAAGACTGTAATATGTGTATACGACCACATTATGAATGAGGGTAAGGTGCGATAGTCA |
| | | GTAGCTGCTTGCTATTATCCTTAAATCGAATAATGCAGCGCTTCAACAATAGATCATATGT |
| | | ATTTCAAGCAACAATTAGGGGATTCAACTAGAGATGCTAATGTAGGTTTGTGAATATTTTG |
| | | GTCGTACATTGGTAGGGCATCTGATTGCATGTATACAGTCATAATTCAGAGCGACGCTCTT |
| | | TTTAACCTTGGGAAAGGCCGTGAACGAATGCGATTAGGCCAATCTAGCGCATATAGTTAAT |
| | | TATTTTACTCTTTATCTCTTGAGCAACAGCGGCAAGGAAACCTGGGAGTTGCTAGACACCG |
| | | AGTAGAAATCCCTTACTTCGCCAGCGGATCGATCTGTACTACATGCATCTTCTACTAATGG |
| | | TTGAAAGTGAAGCTAGTACTTATTTGCATGGTGCACCCATTCTTACAACCAGGTTGTTCTA |
| | | ATGTCTTTTCATCAATTCTTAGCGGAGTGGGCATAATGAAAGTATAAGAATGGAAGTGTTC |
| | | TATTTTGCAACCGGAGACCACATGAAAGGATCGACACAGAGATGCTAACAGTGCATACATT |
| | | CGATGTGGGATAGACCAACTCTTGTACGATTTAATGTGATCTCTGTCACAATTCGTTTAGG |
| | | TGTCTATGGTAAAACCTCAGCCACAACATGTATAGTCTTACAGGCATGGCTATCGTGATTT |
| | | AACCGTGAATAACTTGTCGGTAACAGAAACTCTGGCACAGGTGAGCGTAATCAAATCAACT |
| | | TCAGTAATGAGGACTTCTAAGATAGTTCCGAATCTGTTCACAGTATTAGCACGGTGATTGA |
| | | GTTCTCTTCTAATATTCCTATCTTTACATTGCGTACTGTCACAGAATGCTGTTGCCTCTAT |
| | | GATTTTACAACGGCAATCTAAATCGTCGTATCATATGTTCAGAATATTAAATAGCTCAACT |
| | | CCGTGTTGAGTCCTAAGATAAAGATAGAAACATTGACTATAAAATCTATCCATTGTAAACC |
| | | AGACTAATCATGCAAGCACAAATTAGAGGGCAGACCGCGGCCATTGGAATCATTTATATCT |
| | | TTATCGTTTAATTCACAAGAATGGCTAAATGCCGGATTTTGACCGGGC |
| 317 | 39.10% | TTACATAACGACTCCGTCGAAGCCGTCCCGGACATCGAGTCTGACACTTACAACCCTGAGA |
| | | GCCGCTTCCCTATATGTCTATAGATTGCGAGTGTATGCCACTGTCATTGCAGATTTAGGGT |
| | | CACCCCAAAAACACGAGTATTATTAGAGACTACGTATCATTTAGCAAACAATTTCGCGAAG |
| | | CCCTAATTGAAAAGGCAACCGATTGACCCCTGGATAGATAAGCTAAAATAGTGTTATGCGG |
| | | AGCAATGTTCTCATTTGGACCCATACACTCTATTCCTTCTGAATGACCTTCGAAATACGAA |
| | | TAAGAACATGGCGTTCCCAATCATCCATATACCCGTTCAGGCTGAGTAGCCAACATTTCGT |
| | | ATTCAAAGATACAGTTGACAAGCTGACATTCATTGATGACTTAGGGGCTAACATATCAGGC |
| | | CTTTTCTTAATGTTTAAATACTTGCCTATTATGTGGCCATGAGGAGTGCGATGATACCAAT |
| | | GTTATTGGAGTATCGTTAAAAAAATTCGGTAGTGTTATAATTACGAACTATAGCTTACGGG |
| | | TCATCTATTTTAACATAGTGAGGGCTTCTTCACACTTCCAGTCGTCGGTCTGCATGAAACA |
| | | AAAATGAGTTACATTTAGAGGAATGCGGGGTAGGCACAACTAAACACAAGGATTAAATTCG |
| | | TCGCGACAGGAGTACACTTAACGTAATTAAAAAGCTACCAGGCGAAACTTCTATTTACGGG |
| | | CAATTACGAATCCTATGACACTTCTAGGACCTCTCATTCTTAAATAGAGACAGCCTCCACT |
| | | CGAGCTCCGATTGAGCTCTGCTCTCTTCCAAACAAGAACCTCCGTGCGAGCAGCATATAGC |
| | | GAGCATTCTTCGGAAGGACCTATATAGATCGGTCAGTTGGGAAATCTTACAAAACGTCGAG |
| | | CATATATTATTTGCCGTCCGCAACCTATGCACAGGGGCCTTTAAATCAGTTTATTTAAAAA |
| | | ATCTAATTTCAAACAGTCTTGCAATAGGTTAGGTGGGTATAGAGTATCAAAAATACGTGAC |
| | | TAAAAACAACAGAAGTTGATAAACAACAGTGATTTTCGGGATTTATGCTACACCTTAGCGA |
| | | GAAACTTCTGTTAACATTGTCTATGCTTTGAAACTATGTAAAGGAATTCGTGATATGGTAT |
| | | ACCTAATAGGCCCATACCATTAAACTGAATCATAGTGGACGAGAAGCTTTATCGCCCTCTA |
| | | ATGCGTAGTGACGAATGAAAATCAGACAACCATTATAGAAGTCCGAGTCAGCCACGGATGT |
| | | TCGGAATTGCTATATATACGCATGACTTGCCAAAGTTGTGGTTTACTGTATATTTCGTATT |
| | | CCACAATTACATATAGCTAAATCTACGATCGCGGCGCGGTATAAGATTTCAAACTCGGTAA |
| | | ACTTGAATGATTTAAATCATCCAATTGTTTTATGGATCGTGGCCTGGAGTTTGGCAATTAA |
| | | TTAAAGGATATTTAGCTGAATGTGTAAAATAATTTTTAACCCAAATGTGTCTATAATATGT |
| | | GCTCGGATAAAGCTCAGGCATAACCACAGATCTACGCGACCTTGTGATCGTCCTTGTATGT |
| | | GTATATAGAGCAACTACCAACAGTTGTTCAGACGCAATCAAACGATAGCTTTACGATAGGA |
| | | TGTTCATTTATTACCAAGTACTATTATTCACTCTATAGGGTTATTATATCCTCTACTACTC |
| | | CGGGGTGCGCAACTTTCCTTACGCCATTATTAACGGAATGAGCGGTAAGCGGCACCTTCTA |
| | | TATCATCGTCATAAGAGTGAGATGTAATGTTACTATGCCTTATGCTTGCCATGGTAAGCCG |
| | | AAAATAAGAAGATCACAAAATAGCACCATCTTTTCCATAGATTCTCATAAACATTGATGTT |
| | | TGAGCAAAATAACAGCTATTACAATGATGTAAATTATTATAAATGTCTAATCATAAGCCAG |
| | | TAATTTCGTTAAGCAATCTAGAGAAGTATCTTAAGAGCGTTAAGAACC |
| 318 | 41.30% | CCTCACTGAGACCAATTATGACTTTTCTCTTGCAATTACACAATAGTGCGTTAAGTACTGA |
| | | AAACCATCCTCAAGGCTAAATGTTATAAGATTTTTCATACGAGTGGCGAAAACCAAGTCAA |
| | | ACTGGTTAAATGATGTCTACTACAAGTTTGGGCTTGGCTGACAAATTTTTCTATGAGCTAC |
| | | TGTAATAATGCGTCTTCATACGAACGCACTCTGCCCATAAATAGGCGATGGACCTAATACG |
| | | TCAAGCCCATCTTCAAATAGTTTTTCTTGTAAATTTTTGTCTTGACAGACATGATACGTTA |
| | | ACGTTGTCTTTGACCATTATATCTTCGCGATAGGGTCGAGTTCGTATTTATTAAATTGATG |
| | | AAATTGCGACACATATCACGTGACTTAATCCCGAAAAATTAGAGTTCTTGCGCTTGTCATA |
| | | GGCATGAAAAGCTCCCCTCATAATACGTTTGACCTTTAACGTATGTCTTTAACATATGTTC |
| | | CTGGTAACCAGGATTTAAAGTCATGGTCAGCCTTCGTAAAATGTGAGAAGATCGCGAATAC |
| | | ATCACGAACTCTCTCAGGCAAACATCTCATCCACCATTTATATAGTAGATGCGCTACCCAC |
| | | TGTTAACCTGTTTGAGATGTCGATTTAAACGTTAGAAGGTGGTTCCATCGCTGGATTGCAA |
| | | CCTTTACTTAAGGTCGATGATACGTACAATCGCTTTACTTTAAGCTAAGTTATTGGCATAC |
| | | TACTGAAATTCACTTCCTGGCAGACTTGCGTTGCTCTCGCAATCCCGCAGTCCTTTATGAT |
| | | GTCTAGGCGTTTTACAAATCGACAGTCATTGTATTTAAGTCATTGGATTGTACGGTGTAAG |
| | | TCGACAGGGAACGTGTTGAGTTAATAGTTAAAGGTTCAGATTCTTGCAAGCGCGCTTTTCT |
| | | ATCGCCTGGTTTATCAAACTCATGGTGATTATATATTTTGCAATTCATCAGCCCTCATATG |
| | | TTGGTAAGACTCGGATTGGGTCGACGCCAGACTAACGTCATAAATGTTAGAATTATTAAAG |
| | | ACGCAATTGTTTATGATACTCACTAATGGGTCGTTAGATACTTATTGTTTTAAGGCACCAG |
| | | CCTCCATTTGTCCGAGTCGAGGCCCGAGCTTGGGCGCAAAACTTTTAGTATCTAACTGTGA |
| | | GTGACAACCTTTAGAGTTCTCTCGTATAGAAGGTCCGACGTCAGAGTATCATAACCTACTG |
| | | GAATTGGCCGGGTTCGCGTGCACTCTCACTTCCTGCCAGAACGCAATTAAGCATGCTGGTA |
| | | GTCTCGACCCGGTACCTCACTCTATGAAATGAAACTATAGTATACCTATCGATCTTAAGAT |
| | | GTGGGTTCTAGCTGTGACTGCCCGAAGAAATAGTATTTCAACGACCCGATCGTCTAGGAGC |
| | | GTTGTGGGAGGGTTCAATGCTCTCGTATCGATTCCCAAGACGTTGTGGACATACTAGCTGG |
| | | CGAATAATACTATGTGTAGTGAAGTTTGCGGTAATCTGCGTAGTGGCTAATTAAGAAACAC |
| | | CGAGCCGTGTCTTTTGCAAACTCATCGAGGCGTTGACTAAAATGTCTAACGGTTAGGGCGA |
| | | TATTTTATTTTTACCCGCGGTTTATTATCTATGAGTACTCCCCATTCCCATATAGCGTGCA |
| | | TAGTTTACTTTTCCATATGTTATTAGCAGGCTGTCCGCCCAAACGTTGCGCTAGCCACCGT |
| | | TAGATCACAGTCATATTATCATAACGATTACCAGGTTATAGTTTCACTGACTAAGGAGCCC |
| | | ATAAATGTTCATTTTCACTAGACATGCTATGGGTTTGGCCCGACCAAGATTGATAAACTGC |
| | | GGTAATGGCGATATGATTAAACGATTAAACTTTTAACTACCATGGGGAGACAAGACTTCTT |
| | | AACTAGTCGGTATGGATTGCTGCTTGTAAAGCTAAACAAGCTGAATGTAAGAACAGGCTGG |
| | | CCGGTTCATAACACTATCACGAGTGGCTGACAGAGTTTTACTTATAGT |
| 319 | 40.30% | ATTCGCATTGTTTGAGTAGCCGAGCACTAGTGGGATCATTTACCTTCTCGCGGAAGAGTTA |
| | | CAAAAGTACTGAGGAAATATGTGAATTGTTATAGCTTTTAGGAAAGTAAACATGAAACAAG |
| | | GTAGAACAGATGACGACGTGATACAATTATTTACACAACTGGAAAATTCCGTCAAAGTTTT |
| | | AAAGTATATTCCTTGAGTCCTATTATTGAATATTCGAAAGGTAGTCACCTGAGTTGTCCCG |
| | | TAATAATTACATAAGTATCCGTATGGCAACAAATATCTCCTAGATCCGGGCCGCGGATAGT |
| | | TTTCGCTAAAGTATCTAAATCGAACTTCTTAGCATACGATTACTAGACTATCACCTTGAGT |
| | | AGTCTATATCTCTGCGAGTGTAAAATGCACACGCCGTTAAATCGCCTAAATGCCTTTCCGT |
| | | GGCCATTATATGCCCCACTTGCTTTCAATTCATTCCATAAACTATGATCATGGACCCGGTT |
| | | GCGAGATGTTACAGATAAAGTCGAAACTTTCAAGAGCAGCTGACGACAGGTAAAATTACGA |
| | | TGCACTGCGGTGTAAGGAAATAATCTCCAGGTTGCAATAGACATTTAAATTGTAGAGGAAT |
| | | AGAGTTACGCAAACCAAGCCCAAGGATCTACCGAACCCCTCTACCTTATACAAACTCGTCA |
| | | GCCGAAATATACGAAATAGCACGTTGCCTAGAGGTTTACATTAATCATTTTACACGATCCC |
| | | TTTACTATTAATATATCGATTCCGATCTAAAAGGCGTTTCAAGGATAGCAATAGTCCTATC |
| | | AAAATCATTCAGTTACTGGCAATCCAACCAATTCGCTGTACACGACGGGGTGAGGTCGTAA |
| | | AATATTATATGTCATAGATGCACTGTTTGCGACCATGTCTAGCATTTTTCAATAGCTCCAC |
| | | CCACGCGTTGGCGACCCATTGTTATTCAAAAATGGGCCGCATGAAGAGTTAATTCGTCTTG |
| | | TTCTGACATAAGTGTTGACCATCAGACAATAGACGTATACCGCTGGTTACCTCTAATCGAA |
| | | GATCCAGAGCTCCTTATGCAACGTATAGTAAACCTGGCTCGGAAAGGGGTTACTCTTATTT |
| | | TTAGCACCTACATTCGGGATCAAATCATATGCACTTTCAAGATGGTGCTCACTATAACACA |
| | | ATAACTTGGGTTTCCAGTTAGGATGAGGAATCCGCCAGGTTACTCTATGAAGTCAAGCTCT |
| | | TCCGTAGTTTAGGCGACGCTTGACCCGCGTTCCTCACAAGTAACGCGACAGATTGGAGGAA |
| | | TAGCGACTGCTTCACCATATAGGGACTTACATACAGATCGAATGATTTGCAGCTTTAACAA |
| | | CCCATAACGATCTGCACTAGATGCGATGAGATCTCTGTAAAACGAAACTTGGAATTACCCA |
| | | GAGCAGTTCTAATTAAGCTTTTTCGATAATATTACACAGCAACTAAATGAGCACGTATGCT |
| | | CTAGTGTCGCAAAATCCTTATTGTATAGGAATAGGTCGTTGTCACAACATAGGTCTGTCAC |
| | | CAAACTCAGACATTATAGTACTTTACGGAGCATGTTTAGACATAATCTGCACAATGCTGAT |
| | | TAGTCTCAGTGTGGTCAAATTCTTTAACGTCTCTGTTCCAATCAAAGTGAGCAGACTGATT |
| | | GCATCACAACTCCATCACTTAACCAATTATTAATAGTCCACACAATTCATTCACTCTTCAC |
| | | TGTTCAGCACTCAGTCATGCTCTGGATATTCCATATTTCCCCGCCACATATACTGAGTTTG |
| | | GTCACTCATATGTTCGCTAAAATCGATTTTTAAGCCATTCTTGCCTATTAACGACGGTCCT |
| | | AATCGTTTCCCTTCACCATGGATATACGGTACGGGCCCTATTATCTGCGTrACGCAATGTC |
| | | AATAAAAGATATTCTAAGAAGAAAAAAACATAAGTTGCGTAAGCGTGCTGCAAGAGACACT |
| | | CTCTCTTCGCAGTAAACTAATTTTTCCTTTAAGAATACAAAGCGAACA |
| 320 | 39.10% | GGATTAGATTGTGCCATAACGCAACAGGTAAAATTATTAGACCAGCAAAAGAATCCTAACG |
| | | TATACAATTTTATCGTACATAACCCGTGAATCTTATTAAACCCAGCCAGGCCGCCTTACTT |
| | | TGCTCCAAGTAGGAGCATAATGCATAGAAGTTTCAGTATCCTGTCTAAAGCTATTAAGTCG |
| | | AAATGAGACAAAAGTGACGAGTTATTAACGATCAGAAACTAGTCTAAAGGGAACCCTCCTG |
| | | CGGCCATTTCTTGAGGACTTACGTGCACCATATCATGAGGTCCTACTGTGGGAAAGGAAAT |
| | | CCTCAGTTTACATGATTTGAAATACTGTAGTGACCTGTCAATTTACTGATTTCTATGCATA |
| | | TAATGACAATCTCACCGAGTACGCATAAATCAGCGCAGATCTCATATATTCATAATAATCT |
| | | CCGGGACGTTATTAAATTAATTTTTTTCTAGACAGATATTCAGAAGTCCGACGTTATACAA |
| | | GTGCCCAGTAACATGTTCTGAGCAAATAGATTGTCGACAGCCCCAATTAACCACCTACTAG |
| | | TCTTTAGGCACTGTGTGAATGAAGCTATTAAGTACTAGACATAATGTCATTGCTGGCTCTA |
| | | GCTGAAGAGTATACCTAGCTTTTTTTCCAGATTTTTGAGTACGGGATCTGTTCTTGTTGAA |
| | | CAAATAATCTGGATGGCGCCATACAGGCGTCGCCTGGAGCGTCAAGCTCACATACCCTATC |
| | | GTCAAAGTATGTTCCGTCAAAGGTGTCTCAGCACTTAAATACTTAAACAATCCGAGTTTCG |
| | | AGTTCTAAATGGTTGCACAATATGCCTGGTAGATTGATATAATCTTGAAGCAACGATGGAT |
| | | GAAGAAAAATTATTGATACTTACTTTTACCCACACAAACCGTCTGAGTGTCTTTTTAAGAG |
| | | GGTTACGAATATATAAAAGCGGATCACGATATTCCACCGGGAATAGCGCAATTAGTCATAT |
| | | GGAACATGGTGTGAAACCACAACTATGAAATCTATCCGTACACCAACCAAGAGACCTAAAA |
| | | GTTTTACATAATCCGTTTGCTTTCGTATTGCCCTCTATCTAATGAAAACCCATTGACAATT |
| | | ATAAAGAACAAAGGTTATCACACGCTGCGTATTTAGAGAAGAGAGGACATGTGGGATCAAT |
| | | GTGGTCGCAAAAATTATCACTTTAATCAACACCGATTCTAAGAAGAAATAAACGTCGTATT |
| | | CAAGGGTACTGTATAGGTACGTTAAGCGTTGTCGTACACTGAGCGATTTAACTAACAGCCG |
| | | GGAGAATGCATAATTATGATAAAGTGAATCCACTTAGCGTCTCGAATAGAGGCTATTTCGC |
| | | TTGCAATCAAATGCTTAAGAGTATCCTAACCAATTTTAGACAAATATCAGTATGTTTATCG |
| | | ATTAAGCTGGACAATTCCTCTACACAGATGTTTAAGCGAACTAGCATTTTCATCCTCCCGA |
| | | CTCATAGGAGTCCTTCGTTGCACAGTAGATAGTCAGCGTGTGTTCTCTTCTCCAATTGATA |
| | | TGCTGAAAAACTATAGGTTACCCGTTTCGGTCGGATAAAGAATTTGACTTAATTTTCTTGC |
| | | CGATAGTAGGTATACTGTAAGGCAGCCAATATAACCGTTAGAGCTTGATTAGTATGATATT |
| | | CGCTCCTTTTAATGTATCTACATCTAGCTCTGGAAAACCCGGTGTAGAAGTAATGTATTAA |
| | | GTCTGCGAAGCGGGAATCTGCTTGTGACAAAGATTCTGTCGCCCGCAAACGTCAAGTAATA |
| | | AATCGCAGATACGGTCAGAAATTCCTTCTGCATTTCAAGATTAGTAATCTATTCGATTCCA |
| | | AACATCCTGCTCCTAACAGAATGCGCACGGGACCTAATGAACTTTTCATATACGTTTCATC |
| | | AAGCAGTAGTGTTCGGAAACGAGACATAACAGGGTACATGTGCATCAACCTTTAAAAACCA |
| | | ATCTCTATTTGGTATAGTCGTATTCGAAATCCAGTAGTGAGGTGAAAA |
| 321 | 38.70% | AATTGGAGCCAACCATAAATTGGATGGTAGTTCCAAAATTTTATAACCTATTCTAGTGTCT |
| | | GCAAGTATTTAGGAGATAGGTGAATTACACGTCGTACACATAAATATGATTATGCGATCAA |
| | | GAGTGAATGGGGTCTATAGTAATATGATGTAAAACTTAAGGATATTGTGGACTGATTTAAC |
| | | GTTACGTAGTCCTGACAAGAGTTTAGATGCCAGGTCGTAGAAGTTGTGTATCCCCCTATTC |
| | | TCCCAATGGTAGATACCGTGATAAAAGATAAATTCCTGTTAAGGAAGTCGAGGATGTTCTG |
| | | TGGAGTGCAGAGTTCTACATGTGATGAGATAACCTAAGAGAAAAAGTAATTTATAGATTGC |
| | | CCCCGTTAGGAGCTACACCCGACTATTTGTTTCGTTAAGATATTTGTTCGTACCATGCTGT |
| | | TATAACGACACTCCCTCGAATCTTATTTTATGGCAATTAAAGATGTTACAGGTGGCGTTGG |
| | | CAATTCTGGTAAACTCCGCACTTTACAAATTGTTGTTTGCAACTCTCTCATATTGTATGCA |
| | | ATCGACCCCAAACCCTCATCCTCGACCCTATGAATGAAGGTTTTCTGTGCCAAAAGCCATT |
| | | TTACTCAAAAATTAGCTTTTAATTTGGGGAGCTTAATAGCGAATTCCAGAATCGTTTCATG |
| | | GGGATTAGGAGATATATTATAGGAGTCCACCAATAGTCTATTGACTTAGTGGTTTTGGCTC |
| | | ATGCACGGTGGACAAAACTTCAGGCGTGTTATCTAATTACAACCCGTATTCATACATATCA |
| | | GGGGTGTTGATTTCAGAGAATAGATTAGGAAACTACGAGCAATACCAATTTTGAAGATATG |
| | | GTCTACTAGTAGCTCACTTACTCAACATTGCTACTTTATTCGAAGGCCCATATTGAGGAAT |
| | | ACTGTCTTGTTGAGTAAAACGATACCCGTAACTTTAAACTATAAAGGCATACCAGAAAAAG |
| | | TGTCACCGCAGGAAAATATAAGAACGTCCATCAATATATGATGCAAACTAGAGAAAGAGCT |
| | | TGATAAATTATCAAACTAGCACTTCTGGGAATACTCCGTGGTTGCAAGGTTACAGGGTTGA |
| | | GTCAAAGAGTTATTAAATCGATTGATATACTTATTCAAGTGATTGATTCTATATAGCTACG |
| | | CATATCTGCTGACTTTTTCGATACGTTGCCTGGTTGTCCAGAGCATGTTTTGGACGAGAAA |
| | | TTTCGCGCAGATATCATGATTACGATTGGCAACTAAGGATGACTAGCGTAATGAGAACCTG |
| | | GCTAATTTTGTGTTTCTTATTCAAATTGTATAACTAGGTAAGGAACGACTCGTTCAGAATG |
| | | AGTTCTAATCATAATCTTCTAAAATACTGACAGAAATAATAATATATATTATGACTATTCA |
| | | GAAAACCTATAAAAAGCACTCCGTAGAAGCTCTTCAATCTTAGAATCCTCACCTAGGAACC |
| | | TGAAGATTATTGTATTGACTTATTTTGTAGTTATTAAAGAAATCCAACGACGGGGACGACT |
| | | GCTTGTATGTAATATTTCCGTTCCACAAGCCGGGAGTAATAATAAGCAACCGTAGAGGAGC |
| | | AATGGGTTTTTATCTCACGCACAGGATGTCGGAGTAGCGAGCCGTCTGAGTATGTTATCAC |
| | | CAAAGATATATGTAATATGGTTAATCAGCTGATTTATAGAGAACTTCATCCCAACCTCGAC |
| | | CGACGATCCGATTACTGTTTATCGTCATACCTTACGAGATGTCAGGTCCTCGCACAAACCG |
| | | CCACAAATTCCTTGTCACTGCAAGAATTAGTTTGTCCGCAAACTGTCTACGCGCTAGGTCG |
| | | TTGTATGTATTGATGAGCCCTATCCTTATGACACTCGGACTGCTAGCCTTCTGAGATTTAC |
| | | GACAGGCAGTCTAGTATTAAACCCTTACTACTTTTTGCTGTATATTGCATTGCAAGTTCCA |
| | | ACAAGTTAATGTAACACAAACCGTGATCGCCTCACCCCACAAAAGGCT |
| 322 | 38.80% | GTAAGGGTCGTACCTCTGATCATATTCGATTACTAATAACTCCAGATATATAGAATTGAGA |
| | | AAGGCAAATGTATTTTAAACAGCAAGAAACTGTTTCAATTCGGCTTATCTGATGTACATTT |
| | | AATAAATAGAATGAAGATCGAGTATTAGAACTGATATGAAAGTTCGTAACATCAGGACGAT |
| | | TAGAGTTTATGCATGCTAACAGGAACTGACCTGCTGACATTATATCATACAATTTCCTGCG |
| | | TCCCGCTTATGGATGGCGTCAATAGGCTAGTAACCTAATTGCAGCTTAGAATAAGGAGAAC |
| | | CAAGTAACGACAACAAAATGTAAAGCAATAGATGGCGGACTGCGCTTTAATTGCATTGAAA |
| | | TACTCTGGGCTTCAAGTGTTAGTTCATTAAAGCTGTCTCGCGATACACAAACGCTGCGAAG |
| | | TGGTTCCGGAGTAAATGTGACCAATGTTAGACAGTGGGCCCGCCATGAATGTGAAGTTAGT |
| | | TACTAGGAAGAGTATTCTCAGTTTGGTGTTTACTAGAGGTGTGCTTGGCGTTTATCTGGGA |
| | | TAATAATTGTAACTCAATTCTATTCTTTTTCGTTTTTTCTGCTCATATCGAAGTTTTGCTC |
| | | GCCTCAATCAACGTTGTYTGTATAGCACTTAGGATCACTCTGCGCATAGGGAATGCTTAAA |
| | | TCAGGGAGTTCATCGGTGTCCATCCTGCAGGGACATGAAAGCTGTCATACACGGACTCGTA |
| | | CCGGTCTGACAATCCGCTTTGCCTCATAGCAACTATTGAGCCGCATTCGCGTGGAGCTGAA |
| | | CTATCAGAATGGCTAGAAAGGATAAACCTGTGGTGGGTCCACGAGATTGGTCTTCTTATGT |
| | | TAATATTAGCTCACAAAGTCCAGAGTTAGTATCCATCTCTTCCAGTCACATGGAATTTTAC |
| | | TAATTATTGTGGTATCATTATTATAAAAATGACATTATCTAGCATGACTCCCTACCACTAG |
| | | TGCAGAGCTACTATGTACATAACTCGCTGTTTATGCGATACTCCAACAAGTAGATACGGTA |
| | | ATTTCGATATAGGATGAAAAAACCTTCATAACAGCTTAAGTTTAACTTCGAGGGTCCGTGT |
| | | AATCGGACAACGCACATACGAAGTGGCACGACCTTTCATTTGGGCTCCCTTTTGCAGGCTA |
| | | GTAAACCTAGTATACATGAAAGCCGTCTTGCTTGTGCCTACGGCTTATTTCGTTGAACGTA |
| | | CGTCTAATAGTGCCAAGGAACGAACACACGGCTAGATCATAATATTACTCCAGGTGATGGT |
| | | TTCGGTATTTGCAAAGTAAAGATAAGTTATCTGATTCACAACAATCGAGAATTTGTCCTGT |
| | | TTGAACGCCGAAATATTATCTTACTATTGCTTTACTCAGATACCTCCAATAAATTATAAAA |
| | | TGGCTTGTTTGAATGTGTATCGAAACCGAAAGCTATATCTTTTGACCGAATTAACCAAATG |
| | | CTACGCGTTTGCTGTTTATTATGTCCATCATCGCTTTAGGTTAAGCTTAATAGGTTAGGGA |
| | | AAACTACCAGGATTCACATAATATCCTATCTAGGAAGTTAAATTCACCCATGTATACTATA |
| | | CTACTTAGTCTACAATATTTCTGCTTTATTCTTTATTTCCATTATCAAAGTATTTCGGCTC |
| | | TTAAATGGGGCAATTACGAAAGATATGATTCTAGCTCATGCTCAATTGAGATGAATTTATG |
| | | ACTTTAATGGGGTGTACCATTTAATAATGCAGCGCTAACATAACGTGCGACGCTAATATCA |
| | | TTTACTAATAGATTTTCATTCACTATAATAATTAATAATCTTCTGGCCCCATGGCACAGGC |
| | | AATTTTAAATCCGTACCCGTCAGCCCTAAAATGCCAAGATTAGTGAATCTGGTGTCATACA |
| | | GGACTAACAGGTGCAAAAACCGGTTGCGTCATCAAAACGCAGGATTTACTCAGGATCTTAA |
| | | GAAATCTAAATTTTCGCAGAATCGCTCATCGCGAAAATTTTAGGCGTC |
| 323 | 42.80% | CACGTGGTTTTCAGCGGTTAACGCAATCTGCATTATTGGTAGAATTTTACACTTAACAAAA |
| | | TATCACCACGCGGACAACTGATTTAGCAAATGCCGTCCGTGACGCGGGACCCGCAGCACAT |
| | | TATTAGACATAGTACATCAGCCTGTAACCGATCAGTCATCACATATCCCGGAAAGATTTCA |
| | | ATCCAGTTGTAATCAACGCGTAAAGTTATATAATCACTTCAATCACCTTACTAACTTCAGA |
| | | ATGGCAGCCTAAAAATCTGATGCTACGAACCGCATGGTGTTGAATAAATTCAATAGAATGG |
| | | AGCTCCTGGATATTTCACGACGCCGGGACAGAAATAGTGTTATAGAGAAGAAGGCATGCCG |
| | | TTTTACTCGATTCGTAAGTAGTTTGACGAAGCAAAAACTTGGGGAAGAACTTATGAGTTAG |
| | | CCACGACAACTACCGGGAGGATTTGCTTTTCTTCCTCCATGCCAATCTTGGAGGGAGTACC |
| | | TCAATCACACGATGAATCAGCCTTAATGGGCGCCCAAAACATTCTTGGTGCCAGAAAAGCG |
| | | GATGCTTCCTCGAATGTGTAATCAGAAAAGTGGTAGATGAATCTCCGGCTCCATCATGGAT |
| | | AGAGCTGCAGGTATTGGTGCAGCAGGAACGAAGGTTCTACCAGTAAGTAAAGTTTGACGTT |
| | | AGTTACGAGTCTAGAAGGCCCAAAGGGCAACCAAAACGTCGGCACCATAACATCTACAGGT |
| | | GGTAGGCTAATGTAAAAGTGGTTATAATTGCTAGGCAGAAATAAGGCCGTTCATTGGGCAT |
| | | GTGTACACTCCATTGATGGAGCTTAATTCCTCTCAAAATAATTACATTCTGTTAACAAGAA |
| | | ATAACTTATTGGTCGATCTACGAGCTAGCAATAAATAATCATGACGAAAGAGCTGTGCTGT |
| | | GATCAGAAGTTATGACGCTTATACAGAGAGCATTGTAAAGGGCAGGCCGAAGCAAATTCAC |
| | | AGAGTACCTGAAGCGAACAAAGGAAGAGACTTCTTTATAATTTACATCGCTTGGCAATTAA |
| | | AGAAGCGAAACACAGTTGCTCGAATCACATCCTTACGTGTCGTCGACAATATCATAAGCAT |
| | | TACTAGTTTAGAGAGGTGAGATATCGGTAGTAGGTATTAGAACATTCTAATACCTAAAGCT |
| | | CATTACTATTAGCACCTTTCCTCACCTTATTTGGATTTCCCGCACGCCGTTCGCACCGAGC |
| | | TAAGTGCAATAAGCCATGGCGATGACTTAGATGTCACATTGCCCCATGAATTCACCCCAGT |
| | | GAGTTGAGACGATTTGAAGTTTAATACGTCGTTCGTGGACAGCTTGAATGTTTCACACGTG |
| | | GTAAGTTGCATATGAACATATAGGAGGGGCCACAAAGCTTATGCGTGAAGCAAATATGATT |
| | | CCTCCCTCGATCCGTTAATTAGAGTTGCTGAAGGGCATAAACTTTAGCGAGTTTGTATTAA |
| | | CATAGTCATATGAAGTAACAGAGACCCGTCATAACGCTTGAAAACCTGAACTCAGAATGCG |
| | | CTTTGTGTACCATAGGCATATACCCCACATTACGGAGATGATAATCGACAAATGCTCCAAG |
| | | AAGTAGACCTCTAGCCATCATCACGTGTCTCTACTGTATTCTCCGAAGTTCCGGAGGCCAG |
| | | TTCTTAAGTAGGCACAGAACACACGATGGATTTCCTAGGGACGTACGTATGTTCGACTTCT |
| | | CGTCAGTAATCGCGACAGAAATGGGAAGGTGAGCTTAACCTAACCCACATTTTTGTCATGG |
| | | GACTCTGTGAATGGTGTTTCTTATGAAGCTATCACGGTGTAAAGATATCTAGACACGCTAT |
| | | GTGCTACTCCGATAACCCTACGTTTAGGTTTACGAGATTGGAGAAATATACTTTATTAATT |
| | | CTTCCCTGGAATCGTACCAACAAGTTCCAAAATGGCTCTGCGGTCTGTCAAAATATGAAGG |
| | | GCTCAACTTGACAGGACGACTGACCGGAAATGATTTAAGTGAACCTCC |
| 324 | 38.00% | ATAATTATCGACATAGATGTGCTTCACTCGATTTGACAGCTGGATAGTAAGAATTAGTGTA |
| | | TAACCCAATACGTATGCTAATACAAACCCTGGACTGATTTGAATGTAATCCTATTCATAAT |
| | | ATTTTAGCTACCGTAAATGTATTCTGCAATTGAATTTCGTGTGAATGTAAAAGGTTTAGAA |
| | | GTTTCCTAAGTTATCGGGTGACGTTTTTAATGGGTCTTACCGTAGATTCAGACAATCTTTT |
| | | GGAAACCAACTGAAGAAGGAAATCACACGACCTGGCGGATAAGGGTTTGTAATTCGCGTTA |
| | | AAAAACTGACGTTTGCTATAAGAGACGTTAATGTAAATGTAACGCTTTAAATTCTCTGTGC |
| | | CCTTCTCATTCGTCACTATCCCTCTCCGATCAATCCGATTGAGTCCTAGTGTAGAAAGTTC |
| | | ACATAGAAAGCAGTTTTCCGATTAGTCTAGCGGGGTACTAAGTGAACACTAGTCAGTTGGT |
| | | GATATACTATAGCTAGGCTGTGATAATGTTAATCGGTTTGTGCCTACTGGAATGCTTAATT |
| | | TCATCTTGAGGACTTGCGCTAGGAATCGGTATGTCTTCGTTAAGTCCAAAGTGCCTTTTCG |
| | | ACAGATGTTGGATTGATGCACTCCTCCGAAAAGGAATCAAATTGGGTTTATAAATTTTGTC |
| | | TTTGTGACACCTGCCGAATTTAGATCTCACCATTATCCACAATAACCCTATTATCTTTACC |
| | | TACTTCCGTCGGAGCTTGATTATGAATATTGGCAGAATTATGTAATAGTCATTAATATGTT |
| | | GAATAAAGATATCAATACATTCAGACAATTGAATTAATCCTGCGTAAAAACCTACTTAGGA |
| | | AGCCTGTTCTGATGTGGCCGGCGATCACGTTACCTGATGAGATTTATAGATCTCAAGTCGG |
| | | ATGTCCTCTTTAATAAACTGAAAAATTGACGACTAAGTGGGCTAATTATGCCATCAGAAAT |
| | | AAGCTAACCAAACCTCTAAAGTCGACCCTGTAGTATAACTGGCAGTGCTAGATATCACAGG |
| | | GTGTTTGTCTACTGAAATTTCGGCATTCTGGTCACACTTATTGCCGATAGGTTCTAGTAGC |
| | | TAGTTTATCTAGACTCCAATTGAAAGCTTACTTCGGCCTATCAGGTTGAATGATAGACGGT |
| | | CTGTCTTAAGAAACTACAGGACATATACTGCATCGAATGCGTTTAAATCCTAACGCAGAAG |
| | | GGTTGTTATCTGATCATCAGTAAGCACCAATCTGCATGATTACAGACGTACCAACAACTGA |
| | | ATACATCCTGCCTCCTGAGAACTAGAACCTATTGTATTGCGGATGAGGGTAAGATAGGTAG |
| | | AAACCTGCTGCCAACTTATCGATAATAATTATGAACCATGCGTGGGTGTTGATATAGACTT |
| | | AATATGACCTCCTGTCTGGTTCATATACCAGTTTTCAATGCTTAAGAGAACTAGCTTGTAC |
| | | GGAGTTTTTTTAATACAAGTGCTAAATTAACAATTGTTCAAAAACAGTTTATAGTAGTAAG |
| | | GTATTGTACCAATCGTATAGCAATAAATCATACCTGTGTTTACTCCATACTTTCTTGATTA |
| | | TCGGGCACGAGAAGAGGACAACTCCCAAACATCAATGTAGCCATAGTGAATGAAAAAAGTC |
| | | GGTTATGAATCGTTAGCTAAATCGTTTGCTCCAATTAACAAAACTATAACCTAAACTGGTG |
| | | AACACATAGATAAATGCCAACTCGTTATCGTGTTATGCTATAGATCCGAATTTGGTGGTTC |
| | | TCCGAGTCTGTATCGTTTTTAATCGAGATCTTACCTTATTCCTAACCACATTTCGTAAGCC |
| | | TATTGAAACGGGTATTGCCGGTTCGCCCATCTGGTAGTACGTAAACGA |
| 325 | 38.20% | GAGGTTAGTGATCAAGCGCATTAGCTTTTTACTGCGGAACGCATACAGGATATTTACGCTT |
| | | AAAAAGGTGGATTTCGTATTTATTAAGTATTCTCTTTACTGAATTATTGTCCATCAGTAAT |
| | | CGCTGGCTTTATGAACTATCAACATTCGGTGTTGTGTTAAGTTATTAATGACAGATGCTCG |
| | | ACGTTCCCCAATTCCCGTGCGTGATATATTATCATATGACCATTAAATGATTAAAGGGGCA |
| | | TAATATTTTGAAATAACACTATTAATTTGAAACTTTTGTCCTTTTCGCACTACATGTTGGT |
| | | AACATCGCACGCACTAAATACTGAGATATCGTGCACCATGCTTTCTAATAGCACTCCGTTC |
| | | CAGTCCATAGCTGAGACTGTCTTTTCGGACAACACAATAGATAAGAGTCTATCTCTCATCA |
| | | AAACTGTAAGAAAAGCTCTACCATAATTGGGGCCGAAACGTAATACGATTATTATGATATC |
| | | GCTCCTGCCGAGGTCATACACCATAGCACTCAAAAATGGTATCCAATTTAGAGGGGCTATG |
| | | AGTAGTTAAAAAATAGGAATTAAGGTGGCAACAGGACAGAAGTCAATAGGTTCCCTTGAAG |
| | | GCTAGATTTACAGAACTGTAATGTGACTGCCTGTAAGCGCACTGGAGACATCAAGTATTGT |
| | | ACGAGTATAATTGCACTTTGGAGGTACAACATCGCACTCGACTCTTTCATCGATATTTTTT |
| | | CGTGGGTGAACTTGAGTTAAAGTTGATGGTCCCATTCACAACGAGCGGTTTTCGCGATGTA |
| | | AACGCCGGCCAAAGACAACCTAACGCCGAATTATTCTACTTCATATGCCTAAGTAAGCCCG |
| | | TTCTTTGGAGAAGTCTCATCCTCTATTATTATACATAGTTATCATATTAGTCTAGTCGCCA |
| | | AAGTGTGGTTTCTAATTGATAAATATAATAAGTTAAAAAATGAGAGCTCAAAGTTTTTCCT |
| | | TACCGTGCCGCACAAGTAAGTAGTCTCAAAAGGAGCGCGTAGGGAGGGAAAATTTAATGAG |
| | | TTCTAATATAATATGCAGGCTTGTGAAAGCTGACATTGACTACTCTGGACTGGTCGGATAG |
| | | TTGCTAGACATACCTATTGTGACAAACTGACCCATTATCGAGTCTAGTAGAACCGGTCCGT |
| | | ACAATTACACATTCTTCGTAAACTAGTTCTATAAAGACTAAAAAAATCTATATCACTTGGA |
| | | GAATTATGGAAGATGAGTCAACTCCGAAGTGTGGTCAAAAATATTACAGATTGTATCAAAT |
| | | CGAATAGGCCGTAAACAAGGGGTATACGTTCACAGTACAAAATAAATCAAAGCCTTCAATT |
| | | ATATCGAGAGATTATTACACTACCGCTGCTCTTGACTAGTCAAACGTACCTCTCATTGACA |
| | | ACATTCAGCATGATTATTGCTCCATGTCAAAGACTCCGTGTTCCCATTAGTTTTAAAGGCA |
| | | TAATTTATCTCTTTTCCTCTTGGATAACGAGAGATAATTAGACAATGCTAGTTTCACCAAG |
| | | CCCGACTCGATAAGTGGCGGTTTTAGCCTACCCAATCGCCTAAATATATCAAAAATGACTT |
| | | GTACGCGATAATACTGCTCGGGTAGTTAACGGCCAAGTACACGCTCACAGAACAACGGTTG |
| | | TACCGCTTATCTAATTAGGGAATGTACGGCTCTCTCACTAATATGCGATTAATCTATTTTG |
| | | ATTTTTATGCAGAGCATCCTAAGTGAAACTCTAGATGCCGCCAATTTTTGTTTATCATTTC |
| | | ATAAGTTAATTCTAAAATTCTTTAAATATGAAGACAAACAATGAATTGATTATGATTTCCA |
| | | GATATTTACTTTGGTACCGGATTAAACCCATTTGAACGTCATTCGATATCAAAGTCCGCTA |
| | | ATAAGGGTTTCAATTACAATTCTTCAGGAGAAGACATCGGTAAGCTTC |
| 326 | 38.30% | GCGCAAACCAGCAAATTAGGTTTGACCTTCAACAACTGTAACTCGATCTGCAGACGAGTGA |
| | | GTAACAACAGCTACTGGTACAATTTTTTTGTACCGCAGCATTCAGGTATTACCCCTTCACG |
| | | CTCAGTACAGAGGTATCGGGCATCCGTATAAAAAATTGACTTCTTTTTACGATAGTCCAAT |
| | | AGACCGTTAGCTTCTACTTCATAGTACTAATAATAACCTAATGCAATAGTCTGGATAACAT |
| | | TCACGGGACACTGATACTAGAATCAACTACGCTGATGAGCATGTCCAGACTGACAATCGGT |
| | | CGACATGAGAAGGAATAGAAAAAATCCTACCCTGTTAATTCTGGTCATGTTTGCTGGTCTC |
| | | TTTCCTACTCGGTGCTTCTCAAATGCCACATATTCGAGCATAATACCTAGTTATAGGCATA |
| | | AACTTATTGTTGCTGCCCATGTTGAGCATTTTTTATATTTAGGCCTTTTACGAATTTCTGT |
| | | TTCTATTACTAAAGATGTCAGAGTAATACCACCTTCAGACAGAATCACATGATTAAAACTA |
| | | TAGAATCGGCGGTACAAAGATGTATCTCACCTATAGAGTATGCTGATAAAATCATAGACCC |
| | | TAGACATACTATTCTTATCGCCCCTTAGAAATTATTGTAGGGGTTGCGATTACAACGCATA |
| | | CGGTATTTGCTATATGAGCACTCATGGCTTATGTGTACAATTTATTGATATATATATTTAG |
| | | AGCTCCGGATCGGGTTACAGAATCACTTCACGACCCAGCAAATGCTAATGATTTAAGCGTA |
| | | GTATATTGGCTTTGTGTCCAGTTTTCACTACGGGTTCCTTTCTATGTCCTGATAATCTGTA |
| | | CAACCGACATACCCTGAATTCATGCCGCATATGTCGTGTTAACAGTGATCTAGGGTCCAGT |
| | | GATAGGGTCATTTTCGTATCGTCGCATCTGTATCGATTGGAAAAGAATTATACAGTCCGAT |
| | | TATCACTTAGAACTACACGAGGGGACCTCTTATCTGCCCTACCTATTGGAGTTAAAGTTCT |
| | | AACTGCTCAATCTCAAGACGGCCGAAGATGGTTTTAAAATGACGGTCCACACATTTACAGA |
| | | CAAATTGGAATGCTTAGATATATCCTACTGTTGATTTTTGTCCAAAATTAGAGGCGATGTA |
| | | ACCCCACTGAAAGATTGAGCAGTACAGTAATTCTAACTTGAAAAAATAAATTTTTGGGTAT |
| | | GCTCAATCTTTAAGGTGACCTACTAACAATATCCTAGATCCCATACGGTAGTTCGACAGAG |
| | | ATCCAATACATTCTAATCGAACATTAGTAAGTTAAATAATATAGAGCTACATTTCTAAGTA |
| | | AATCGATGCTTGAAGATATTGGTAGTTCGCAGAATTTGCATCCATCACAAACACTAGTCTT |
| | | TACGTTTGCCAATTGCTAGGTAGAGTAGATTACGAGTCAATCAGAAGACCAAATTTTTTGA |
| | | CCCATAGGATACAACACGTAGTCATGACAATCGCATATCGCTAGTATGTTAGATCTAAGAA |
| | | AATAGTCTACTTAACCGGGTCATACATCTCAGCTATTAACGATATTATGTTGCCTTATGTT |
| | | AGACACGTCAATAAGTAGAGCATGCATTTCTGCCTCAAATAACAAATTTGTTAATATGCAA |
| | | TGAATACCTGAGTTGAATGAACCCAAACTAAACTCAGGGTCCTTCCATAGCGAGAGCGCTA |
| | | GGCTAACATGAGATTCTGACGTCTTCGTGAGTTGACAGGATCTTGCCAACAAATTACATAT |
| | | TTGAATAGGCATGTACGATCCATTATACTATGAGTGCCAGAGAAAACTCTGCTGGCCGACC |
| | | GTTTTACGGGGGGAAAGTCAAATATGTAGTAAGTACGAATTTTCCTGGGAGACTATAGTTG |
| | | CTGAACGTTCTTATTCTCATTTTCTTGAAGTTAAGGATGGTAAAACATACTATACCTATGT |
| | | AGATATTCTTTGGTAGTATAACTATTATAGTAGCGTAGACGTTATGTG |
| 327 | 39.50% | GCCTAAAGACCTCTATATTTTAAGCTAGCATAAAGGCAGGAGACGTTCTAACATCGCACCG |
| | | AGTTCGACTATGAAGAGAGGTATTATCAACCCTGTCTCCCAGTTCACACCGGTTGCATTAT |
| | | CATGACGTTTTTGATTTGTTTTTTTTGAGTAACGGGTTCATTGTACGTTCGATAGAGTACT |
| | | CGATAAACGACTCATTCCACGCAAGCCTATTTTGTAACTTATAACTAGACATTAGTCTATG |
| | | GCTACTTTCACACCCGAACTTACGAACAACGAGTATTTTTTTTTTGGCAAAAACGTAACGT |
| | | TCGTATGTGGCCTAAGTCATTAAAAGACAAATATTGAAGAAAAACCCATGATTTAATACCG |
| | | ATAGGACATTACAAGGGTCATTAGAGATAACAAATAAATTAGGCTTCTTCCAAGAGTTATC |
| | | CGACTAGTTGTGCTCCAGATCTGCGATACTGATCGAATTTATACCTCATTAGACATTCGTA |
| | | GTCATTGGTGTTGGACTTGAAGTTCTGTACAATCCTCGGTGATCACTCTTGGACAACCTGC |
| | | TGATAAAACATGTCTATCGTCAGTCCAGTTTGTATAATAAACTAATGAGACAATATACAAA |
| | | AGAATCCGTGGCACTACATGTTGTATACCAACATAAATTCTGAAGACCTATGATTCTTGTG |
| | | GCCGAATAGTCAACAGATTTTACGATCACTAATAACCATATATCTGTTACTTGTCTTCTCA |
| | | GATAGGAGCGGACTAGAAATACTCACTTATGTTATTCTTACGTTACTGTGCCAGACGAGAG |
| | | GTTTTTGCAGACTCTATGGTTTGCCGGATCTTGCTAGGAAAAGGGTAACTGGTGCGTGATT |
| | | GCATGAACTATGTGGTATGACTATAGATGAAGCATCCGTCACTGAGCTCTTCGAAGTCTTT |
| | | TATGAGACAAGAATATTCTTTGATAGAATCATCTATGTCTCAATTTAATCAAGGGAACGGT |
| | | TGGGTACTAAATCGAGTTATCATGAGGTCCTATCGGAATGCATTGTATTTGAGCAATATCT |
| | | ATAACTGTAGGTACTATGGCGGATATTTATTTTCCTTGCTGCGACTTCATGTAGGAAGTCG |
| | | GCAATTCCCCGCGGTTTTACATTTTCTGCTTCGAGGTATTAAGGCCCTAAAGTTGTATATA |
| | | TTATAAATTAAAGATCTGGATTATTAACTCAGTGCAGAGGGCGTAATCTGACGTGGCGACA |
| | | TGTAGATGAAGCTTGCCGAAAAGATATGAGATCTTAATATCTATAAGAAGTATGCCTACTG |
| | | TTAATTTTGGGGAGAAATGCTACCCCGGACAATTATGCGATTGTCAAGCGAATATCTTGAT |
| | | TTTATCCTTGGAATAGGTATATTACTTCGGTTACACCAGATATGAACCTATCTATTACTTC |
| | | ATATTTTACTCAGGCTTGGTCGGGACCTGTGTTACTTTAAAGGCATTAAAACATACAGCGT |
| | | CGACAATCCTCCTAATCAATATCCTCAGTAGGAATTTACTCGCAATAGCGAACTGAGTTTT |
| | | TTGCCTGTACAACGGTCGTGCCTACTCAATCATTGCCGCATACTAATCTCTATCATATTGC |
| | | CTTTACGGGGCGACCAAGGAGGAATCCTATCTAATCCCAGGGCACCTGGAACACCTGCGGA |
| | | ACATGCTTCAATAATAACATCGTATAAGTCTATGTCTGCGCTTGTGACGTCATAGTACTTC |
| | | TTCTAGTGATATATTACGCCGTTGGATTGGGATCACGTTTAGAACGACACTGTGAACTTCT |
| | | ATATGTACTCTTTTCTCACGATATGCCGTCGAGTTTTTTATCGATAATAGGCAGTGTTGGA |
| | | GCGGGACGTGTCATTAGTAATAAGTTTTTCCTATCAATTTCCTGCGATACTTGACTCCTTT |
| | | GGGGCAAACATAGACGACGGTTGGAGTCAAGGTGAACCAAAATAGAAGTACCTGGGTAAAT |
| | | GCTTCATAGGCACTTGGACAAGACATTAAGTCGACACACTATGCCTTT |
| 328 | 38.10% | AATGTTCGGTCCCGGGTAAGCTATCATTCTATAAAAGTCCCACCCCGCTTATTTAAGATTC |
| | | ACAGCGCCGCAATGACGCGGAACAGGGTTGTCTATGATGACCTAACTACGGCACTTTAGGT |
| | | ATCATATATTGAGTTGAGCGAATGGATCTGCTAGGCTTCCCGTCTATCGGATGCTTTAATG |
| | | CAGGTTAATGGCCCGATTGAAGTTTATAGTATATATATACACTGTGATGGTGTAACTACGT |
| | | TACTTCGTTACTGATCAATTTTCAAATTATCTCATTTGTTAGGCTACAACTAGGACTAAAG |
| | | CTCAAGTAACCGATGCGAAGAGGCCGAGATGGTATAATCAACGGGGGTGTAATCTAATATA |
| | | CGAATCATGCTAGGAGAGCAGCTTATCGTCAAAACTCTGTTGGCCAGATTCTAATTACTCT |
| | | TTATTGTATCTTTTTTCATGTAGATTAACCGTGAAGACAGTAGTTCATGTACGTTAGTCAA |
| | | TTATTGAGAACATTAGCTTGAATGGACGCGTGCTCAAATAATACCCCAGTAATCTAAACCA |
| | | TATTGTTAATCTTTTACAAGACCCACCAATGACCTAATGAGTTCACCTCCACATACCTGTC |
| | | ATTAGGTGACCTTATTTCCACATTTGTATTAAATACTAATAACTGACCATATTGTGCTGTG |
| | | GTTCTGTACACTTGTATACCTGTTCGGCTAATACTAGTCAGTGATTTCATAGCGAATATAA |
| | | CATTTGACAAGACTGTAGCAACAAGTTTTTGGTATAGGGTTTGTTAAAGCATACCGCGCAG |
| | | GACGACCGTCTCTTACATTAATTTACTCGTTTTAATCTATAATTATCCATATAATCAACTA |
| | | GTCCTGAGCCAAATCTTCAATTTCCCCCGCGTTTGAGATTGCTTGATGAGGCGAAATAAGA |
| | | GGCGAACGGAACTCCAAAAAAGAGCGATCTTTTATCACGTCCCTCCATAACGCTTTATAAG |
| | | TGATTAGTCGGCATCGTTACAAATTAATGATAGACCAGAAAGTACACAGACGTGTCTTTTA |
| | | TCCTGTAACGACCCTAATTCGGCACCGTCTACTAAATGCTTTGCCGTACGCTCTGATGATT |
| | | CTATCCAGCGATTACGTATATGTTCCGGGGTAACTACCTAAATCTAATGCGGCCATAGGCC |
| | | CATACTGATCCGCCGATTTCGCGCACTGCTTTACTTATATACATCAGTACTACTCGGGCAA |
| | | CCGGTAAATAATTTACAATAGAAGTTTAAGTGCAGTTACATGCTTAAGATATCGAGAGAAC |
| | | TTGTGAAATACGTACACTAGGATTTTCTCAAATTCGTGACATTACAAGGTCTGGTTTCGCG |
| | | ATTCTCTTGGACTGATATAATATGATTGAAAAATGTAGTAGATATGATCCTGGATAACATT |
| | | TTTAAACAAGTCTTGGGTGAGCTCGGTACCTTAAATCCGATCATAGAATACAACATGGCAC |
| | | CTACATTCATATTAAATAGTCTATTACATGATAAGACTCCTTCATGTCTGAAACATTGGTT |
| | | AGACAATTCGCGGTTTCAGTGGGTAGCGTGTTCTATTGACTTCGAAATGAGAAAGTGTTTC |
| | | GGCGCGTACGGTATATCTTCCCCCATGATTATACATAACATCCTTCTAAAAATCGCGCCAC |
| | | TGCAGGGTCCTCTTTTCTTATATATTATTGAGGATTTGGACCGATCAAACTTAATATTAAA |
| | | TATGATTCTACATACAAAGGTAATGATGGCAATCTACTTGCGGGCTCGACTCGTAGTCTGT |
| | | TCAATGAAAAATACATTTCTCAAGAAATAATCTTCGAGCTATTTCACTCTGTAGTTAAAGT |
| | | TTCAATCTTGTTACATACTGCTTATACAAATTTAATTTAAAAGCATGTGTCAATTTAAGGC |
| | | TAAATGCTCAGTGTAAATTGTATTGGTAAACTCCCTAAGACTAATGAATAACTTGATAATG |
| | | TGGATAGATTAAATCCGTGCAAGCCTATCCTAAAATCAATTTGAAGTG |
| 329 | 41.00% | TACAAATTGTCCACGGGCGTGAAAACAAGCCCATTCTTCTTCAATTGCAAGATTTGCGATA |
| | | CTTAAACCTTACTGATTTAATAATCGATTCAAAACGCAAGAGTCATGAACAGAACGAGACC |
| | | CCGCCATATTTAAATGCACATTCGTGCAGCGATGGGTATATTGAGGCTGTGAGAGGCTCAA |
| | | TTAAACATTTTACCAGGAGATGGGCAAAATAATGCGTGGGGATCGCGGGACTATAATCTAA |
| | | TCAGTCATACTCTAAAGTGAGCTTCGTGATATCTTGAGGATAAAAAAGGGCCTAAGCGGAC |
| | | AGGGTTATTGAGTTCCAGCTAATGATGCTCGATAATAATCGGCCGTAACTTCAATGCGAAG |
| | | AGAATATACGATTCTGAACAGTTACAGATAAGGCCTATTAGGCGCGAAAATAGTCGTCTAA |
| | | AAGAGGAGAACTGCTGGTCGAGAATGAGTGGGGGTTATTCTAACAAAGGTAGCTAGGTGTG |
| | | GTTATAAACGAGAAGGACTACACCCAATTGATCTCGATAATAGGGCGGGATTGTTTATTGA |
| | | CAGTAGTGAGGTGTTCTAATAACAGAAATTTAGTTAAGGTGCGTATTCTTGGAGTAGAGCA |
| | | CAAAACCCGCTAATGAGCATTGTATGAATCCGCGACAAAAGAGCAAAGATCACAGCAACGA |
| | | AAGTCTAATTGAAATAGTCCTCGATTATGCCGGTGAGTTGAAAAAAGTTGTACGTTCGTTT |
| | | ATGCCGTTCTAGATAATTTACACATCACATTCCTCACGTAACTACATGATTTACCTACTAT |
| | | CACTTCCAATCACCAACTCGGATTTAGGAATACTGTAACTTATTTCCGATTATCCGATTGA |
| | | GACCTAAGCAGAAAAACATAAGATGCCCATCCGAATTGTGATGTGGATACCAGTTGTGATA |
| | | ATTCGTCGGATTGAACTCAGCCTGCTTACCGCTTTTGATCGCAGTCGCCGCGGGTAGATGT |
| | | AGTTAGCCTCACCGGCTGGATACATATCTCCAGGAAATCGCGGAGTATCAATCTCTAGAGT |
| | | AAATCCCCTGCCTTCCGTTGATCGTCTTGCTCACCTAAATGTCTGAACTAGGCTGAGAACA |
| | | CAACCATACTCCGGCCACGTAGACGATGCTGAATATTACGCAGCTATACTCAAAGTTAAAC |
| | | TCTTCTCAGTGATTTATGATGTAGCTTAGTGATCTTTACAGATTTGGTATCGATTGGGAAT |
| | | CCAGTTTAAAACTGAAACGACATATAGAAATATGTACCAATCTACCAGCGCAAACCGAGTC |
| | | GAAGTCATATTATACGGTAAATCACCATCGTGTGATATATTGCAATTTGAACTGATTTTTA |
| | | ATCCCTAGCTTAAATACTTCATTGATTTCTCGCCTTTAATTCTCTGAACGTTACAATTTTT |
| | | CTGCCCAACGGTCCTCCTCTAGAATACCTCGAGAGCCGACACAAATACAGTTAGAGAATTT |
| | | TTGGTGATTTGTGCGACTTATTAGAACCACGGGGTCATGACCTTAGCCCGAATAGGTAGTA |
| | | TCCGGATATCTGAAACTCCAGGCAGTAATAATACATTGCCGGAACGACAATCGGATCTAGT |
| | | GAATGCGACATAGACGGTAATATGTTAAGCACCTCATAGATGATTACTATCAGGAAATATC |
| | | AATTTAAAGCTGCGATGAAAGGGTCAGGACCCAGCCCTTTCAAGTCTACGTAACTCCACTA |
| | | GCCACATTGTCTAAGGGTGCCAATCATAGATGATGCATCAACACCGGCGATACGCTTGTTC |
| | | AGGCATTCATATCTTATAGTTATAAAATTTGTTTATCGTGTGCAGGGGTCGATTTTTCTCA |
| | | CTTTCGGCAACCAGGAAAAGTAGTAATTACTATATAAAATGAAGGCGAATTTCGGATTACT |
| | | CTGCAAAAAATCATTAGAATACACATCTAGGATCCGGAGGTATCTGCCTCCATGAAGTTAA |
| | | CTCCATTGTGGATATGATGCGAGTAACATATTTAGGTCCGAAGAAAGG |
| 330 | 39.30% | ATCATCTACCTAAGACAGAGCTGACCGTATCCATTGTCAATAGAACAGCAACGATTTTTTC |
| | | CATCGCTGGAAGAGTGATGCGCACTAGTTCATTTCGGACAAGTAACTTGGACGCGATACAA |
| | | GATACAATCGATGTCAGAGCCTCTTTAGTACATACCATGGAATTATGAATCGACTAAAAAC |
| | | GCAGACGTATAATTCAGCTGATCGAATGATTTCGATTATATACCGAAGTCAGTGACGAGAA |
| | | CCTTCACTTTGCGGGATACCGAACTCTGTCACAAGAAATAAGTATAGGTTAGAATCCAGAG |
| | | AAAACATTGAATATTATGTTTTTTCGCACCAAAATAATCCAACGATGTTACGCTTAGTTAG |
| | | TGGATATCATGACTTCACTAAACACTTGGATTGTTATCTAAAGTTTTTATCTTCCTGGCTG |
| | | CGACATTGTTTATTTAAGACGTAGTTAAAAAAGTCGACCACGGAGGAGGAATTACATCGTC |
| | | GCTGATGAGCCCATTTTCGCTAAATGCAGTCGACTACGAAGAGTTTTTCGCGTATCGTCAA |
| | | CATAAGTTGATCTTTTTAGATAACAAACAAAACTCTTCGCATCGACGTAAAACATTTTTCA |
| | | TAGGCGCTTTTTACACCGAAGAATCTCAGCTTCAGAATTGTACGATGTCTTGTCACAGATA |
| | | TCCTTTAAACAAATAACTAATAGCGTTGATTGTTTGACATCTACTCCTTATTGTTATGAAT |
| | | GTATACCATATTGTTATATGCTATTAAATCCCACATATTGCGGTTCGCACTAAAATGAACA |
| | | TCTATATAACTTGACTGTTACTTGAATTAGTTATGGTCCAGCTAATTTTTCATTCTAGGCA |
| | | TTTAATCCTTTATGTTCCATAGTTTCCTTCGACGCCTTGAACGATGGGTGCGAGTCCGACG |
| | | GACTAACATTTATAAAGAGATTTGTGGGTTTGGGTTTGCTACAGATATCTGGACGCAGGAT |
| | | GTTTAGAGTAACATCTGTTGTCATTTGGCTAGCAAAATTTGAGTTACCTGATAGACCTTCC |
| | | TCATTCCCTTAATATTAAACTGTCTTTCTCGAATACCGTTCGCACAGGGTCCAGGAAATGT |
| | | GATGTTATGACGGCGTGCAATGGTTAGTCCTTATGCAGGAGTTTCTCCGCACCCATCAATG |
| | | CCATTATTTTACAGTCAAAAAAACATAAACTTGTATGACGAATGCAGACCTTTGAACTTTT |
| | | GTTAACCTACTTTTGTAAAACCAGCGAACCCTAACAGTTATGTAACGAGATCCGTTAACCA |
| | | AAAGCGGTTATCCGAGGATAAGCTTCCTACGACGTCACATTTGTCATCTTCCTTACCGGTA |
| | | TGAATTGTATGCAGGTCCCTATTCGAAATGTGGTTATAACTGATGGGTATCAGCAGGTTAT |
| | | TTATAACGCGTACTTTATCCTTGTAGGTTAGTTGCTCAGTACGCCCAAATCAAAGAGGAGG |
| | | CCGAGGTGCAGGAAGGACCTGACTGACAATCGTAACTAAATTATCCAACAGGATTGTTAAT |
| | | TGACAATGTTTACACTGACTATGGCAAAAATTGTCTCCCAAACGGCTGCGGACAGCGTTCT |
| | | TTTTATCGATCTGAGGTAGCACTTGCATATGGATATAGCAATAAGAAATAGGGAGATACCA |
| | | GCGAAGAACGGAGTAGATGCCTGTGACGTGTGCCGACCTGACATTGATTATCGAGCATGCG |
| | | GATTAAAATTCAACAACTATTCCCGTGAAGAGTGCCAGCCTGTAGTCAATTATTGTGGATA |
| | | TTATCTAAGTTCAGATCATACCTCTCGTCGGTGAAAAGAGATAGAGGCCAAAGGGCAAATC |
| | | TATTGAATGATTGACAATTTGATCATATACGTGTCTAAGAATTAATTGTAACGGATGCGAA |
| | | TTCGTTAATCTTCCTGGGGTACTCTTCTCCACGTCACGAGAGATAACAACAACATCAGGCT |
| | | TCTGATAAATAGCGTAACAACGTATTATCAAATGCATCCTGTCTGTAT |
| 331 | 38.10% | TTAATGACCCCTGCCTTACTGCATAAATCTCCTAATTGTGTAATCACTCCTCACTCAGATA |
| | | ACGCTTTACGTATGGATTACCAAGTAAGTGAAATCACTATACAAGAGATTGCCTAATTTTG |
| | | CTAAGTTAGCGTTGTTCGTGTTTTATAATTTTATTGTGAGTCTTTCACCGAAGTAGAAGGA |
| | | AGTAAACTCGCAGTTTCTTATAACCACTTCTAGGCGATGTAGACGACATAGAAAATGGGGT |
| | | AAGGAACTCATAATTTTTAAGTCAATGATACAGCCTTAAAAGATAAAAATTAGATTACCGT |
| | | TTAATGAGGGTACGTGACCATTAACAGTAAGAAAGCCTGCAAGCATGGGACAGGTGCTATT |
| | | GCAGAGCTCATAAACGAAATGTCGCTTGGGCGTCCTGCACCAGATACTTAGTGGCGGATGT |
| | | GAATAGCGAGGACGAATCATTGGATGAATATTAGCTAGTGGATACGGAAAAACGTGACTAC |
| | | GATTGCGGCATCGAGTTCTTAACCCTCTCATGGAGGCATCTCTCGACCTTACACAGTGAGA |
| | | GTGCATTTTGTTCGCCAGTCTACTATGACACATTAAGGCTCAAACACGCTCTGCTTATTCA |
| | | TTTGGCCTTGGGGTTCTAGATCACACTACAATTGCCCTTTGCAAGAAATACAAATGTCATT |
| | | GAAAAATTAACTGCTGTCTTATAAACCTAAACTACCAGATACTGTAATTGGTTTTAGGTTT |
| | | GAGCATCCACCAACACCAATAGCCAAGATTGTTAAACTCTAATAACTGTCTAATACACGTG |
| | | CATATTCATAGTGAATCAGTGCGGTTCATTTTCTGAAGAGCTCCAATCTGAACGATACAAG |
| | | GCGTCCTGCGCGTGGATTAAAAACAACTTAAGCGTTACGCAGAGCAGTATTCCATTTTATA |
| | | ATATACCGTTTGCCGCAGGAGGTTATATTGTAGAAGATTAGTTCATTTTGTGGGGGATTTA |
| | | CAGGCCAATATTTACCAAATTTTACGAGGTAGTTGAACCTAGTGTTACTTCGTGAGGCTCG |
| | | AACGGTCTTCCCGCTCCAACTGTACCTTTAGATGGGGGCTTCTTTGGATGTAACGAAGTAC |
| | | CGGCTTAATATGAGACGTTTGTACGCGAGGCATTCTTATTTAACCCATACTTAATCAATTC |
| | | AAAATTTATCTTGGTGAGTAGCACTGGAGAATTTGGTATCCATAGCGGACCGATAGAAAGA |
| | | TTGTTATACCAAAATTCATGAATGACGCTTAGTATTTTCTAGTTTGATAACATGGTTAAGA |
| | | CTACATTCTATCCGAATTCTTATTAAAATTGAAATGACGCATTGCATGCTGTGATTCCAAA |
| | | ACCATGCCGACAGGAGGTCTTCTTAAAAATTCAGCGTGAGGTTACTACACCTTCAAAAGTG |
| | | CATAATTGGTGGACAACTAAAGGATAATTGGGTAAGATCTTTCTACATTCCATTAAAAAAT |
| | | TCTAACAAACCCTATCTCATGTTAAGTACTTATGTTGCCTCTTACTACATTGACCCTACAC |
| | | TCAGATATGATAAATTGATGTTTAACCTAACTATTTAAAAGCTCAATACCTTCCTTTTTAC |
| | | GCGCAATAAAAGGTTAGGCACTTTTAATGTGAAATTTCAGCGAAATATTCGATCTTGATAT |
| | | AACTAAGTTTACAGTTCCTATTACTACTCATTATAATAGAATGTATGGGCTATGAATAATA |
| | | AATGGACCCTTAGAAGGATAAATGCATTGATTCGATGCTAGAGTAAACTGATGGCTCAGAC |
| | | AGAATCATGCCCATGGGGAAACATAACACCTAATCAGCATCAACTAAAAGTCACATGTACG |
| | | AGAGCAGAATCAAATACAAATCAATTATATAACGTGAACGTAGAATCCGGACCAGGGACGT |
| | | TTCTACTCTGACTATATTACCGCCAGCTGCTATAGTAATCGCGTATGGAGCATGTATTTGC |
| | | TGACTAATGCTAAAGTACAACATTACTGTGTAATTTAAAATGCTACCT |
| 332 | 40.40% | TGTACTTGTCTTCTTGTTTGTCACATACGGACCCTAAATGACCTTGTCTAGTTATCCGATA |
| | | GACCTTGCTTAAGTAGCCTCCCCTAGGGGGAACTTATTACGGAATAACAGTTTTACAGTAT |
| | | TAATCAAACTCTTATCCACGTTTTCCTGTGATCACAACGTATTGTTTCCCTTGATTTGTTG |
| | | AGAATCTCTATTGAGCCTTTTATCTATTAGAGTCTCCGTCGCACATAATCCCGGTGCGTTG |
| | | AACAGATACTGGCTAGACTCCTTACTTTTCTATCAGTTGAACGGAGGATACGAGCTTCAAA |
| | | ATAATGATTTGTTTGTAGATGTCAGAGCATCGTCGTGAGAGGAACCCGGATAGGGGGAATA |
| | | ACAGGTAGCGTTGCGGTTGCCTGACTAAAACCCAGGACTCAAGTTTCATTATTAACATTAT |
| | | TTGCATGAATGACAGTGTCGCAGATCTGGTATAATGACCAACGATCGTTTAGTAGATAAAT |
| | | TCCAATCTAACAAACACTAACCAGTATCTCAGCCCACATTGCATCTTGTTTTAGCAATCCT |
| | | GCAGATATCAGAACCCTCCTGCAGTGAATTGACTAGTGCACGACGGTAACATATCTCTTTA |
| | | ATAGCGCACCGTCCTCAACGTAGATGTTACGTCTGGGGTTATATTGGGCCGGAATGTCCTG |
| | | GGCTTGGACTAATGAAGGCAAAGGCTATAAATGTGCTTATTATTTACTTCTGCGTACTTAT |
| | | TTGGAGAATGTCATATTAAAGATGTCGCGGTGGTCGGATTAATTGAATAATGTGCGACTTG |
| | | GATGCACCTCAATCTTCATTGTTTTGAAAAGTCTGGAGACGTGCAATTACACTCTATATGT |
| | | CTTTGTATTAATCGTTATAAGCTCTAAAGGAGATAGCAAGCTCGGGCAAATGGTAGATTAA |
| | | TGCTTCAAGAAAATACAAGCCTGGGGATTCACATTCCGAATATACAACTAATGACGCTCTC |
| | | ATTCTCTTGCAAGTATAGTAATCGGCCCGCTACTCTATGGGGAGTATGGCATCAGGAGAGA |
| | | GTATCATTGACATTCGAAGTTTGCATACTGAGCAATAAGCGGGTAATGCTTCAAAACAAAG |
| | | TGCACTCACTTAATGTCGGACATTGTTTATAAGTGTTAGCGCTCAATTTTCCGCAATCACG |
| | | CTCGAGCACTAATAGTTGGAGTTCGCTTTAGTTTGATAATAACAAATATGACTTTGTCGCG |
| | | AGATTGCCTATTTGCATCCAGGACTATCGAACGCAACAAACTCGTGAAGAGGCCGCATTTT |
| | | AACTGCAGGATAGTAAGATCTAATTATGAAATACATAGTCCAGAAAATCATTCGAGACTAC |
| | | TTAACAAATAGTTTCAGAGGTTCTAGACTTTCTCAAATGTATGTAGTTCGTGAATATGTAG |
| | | TTATACTCAATTACGACTTTGATTTTTATTTACCGCCTTAGAAACTTGATTGAAATAATCT |
| | | AGAAGCCTCAATCCTGCTCCATCACAAACATAATATACTGAAAGCTAGAGGGCGTTACCAC |
| | | AGTGGTACGTCTAGATTCCAAAGCGTGCTAGGAGATTAGTGGTCGAAACGCAGGTTCCGCG |
| | | AGCAGTATCACCCTACAAAGTAGCTGGTTACAGTCAAGACCTAGCAGCAATTTCTTCACTT |
| | | TTGTTACGATACGTCCGTGGCATGATCGTCGTTGCCTAATTCTACGACTTAAAGATACCGA |
| | | AAAAAGCAAAATCTAGAACCATGATAGAGCTACAAAATCCCTCTACCCGTTCGTACGTGCT |
| | | TCCTAATCAGATCAACTATGTGAGCGACATAGTTTTAGCTAGTACTTGAGCGGGAGTTTTG |
| | | TTCTCGTCTCTGAATATATAAAGTGTTTAATGAAGTGCTATGAGGGCCACTCATCTTTAGC |
| | | ATACTAAATCATGAGACATAAAGGTCACCCGAAATAATCAAGCAGAAGACTAACAGAACAT |
| | | GCTAAGAGAGGTCTTTGAACTACGGACTTGATAGATAACCGTTAGCTC |
| 333 | 40.40% | TCACGACGAGTGAGGTCTGAGACCGTCATCAAAGATCGTAACACTTTTTACCGGGCTGCCA |
| | | TAACGTAAGATGCATGACTGCAAGAAAGTTCACGGTGGTAATTTCAATGAGTCATTGTCAT |
| | | TCCCTGAAGGACGTATAATACTATGTTACGTAGATTATTAGGGATCCTTATGCGTTGAGGA |
| | | GATATCTTGCCTTGAGTGAAAGAAACTCATCTGTTTAGAAACATACCAAATATGTCAGACA |
| | | CGGTCGGCTTTGATAAGAGTCCCTAACTAATTGGCTGCACATTACGATTCGCCGAAAATAT |
| | | ATGTTGGGAGTAGTGTACACGATTTTAGACAAATTCCCGAGATGATGACCGTGACATGTAC |
| | | AATCGCACTAAAAATCCCCGGTATTAGACTTTGAAGTGGTTTTGGTATGTGATCTTAAGCA |
| | | TATTCACTATACTAGCATAACAATGGTGGTTGCTTTTGGACGCAAGTTCTGAGTATATGAC |
| | | TATGAAGCGGAATCGATTAATTATGTCTTCCAATAAAGCTTAGAAGTATGGTTCGTGAACA |
| | | GCTTCCAGTATAATTTAGAGAGGCCGACAATATATATAGGGTTTTATTTACTATTGGCCAA |
| | | GAACATCCTCAGTCGATCTAAACTTCTTCCAAAGCACTAATTCTATCGCAAAATGGTATTA |
| | | TAACAACACTAATCTTGGAGTCAACTCATATACGCGCGTGTAGAGTCATGTAATACTCAGC |
| | | GGCTAACTACATGTATTATGTCAAGTCTTCCTTGCTATGAATACTGGTATTCCTTTGTGGA |
| | | TTAAAACGGTACCGTCATGTAATTTTGAGATAAAGATCTAGGACGGGGAAGAAAATAGTAA |
| | | TACGGTATGTATGCGTTGAGTTGGGTCTGGATATTCAGTCAACTATGGGTAACTGAGGACT |
| | | TTGACGCTGCATCCCCTGCTGGTGCGTAGTCCTAAAAAAAATTCTCTGGGACAATATGTCT |
| | | TCACAAGATCCTTGTGAGAATCCCGCTTCCGGTCCGGCTGGGCCATATAGACTCCTATTAC |
| | | TTTCAAACTTCGCACAGAATCTTAAATATGAGATTGTAAGGAAACTATCAGATCTGCTCTA |
| | | GACACCGACGGAGGAGCTCCCGGAACGTTCCAAAGCTTTTTTTTCTAAGTGTTGCACTTGG |
| | | CCGGTCGTACACGCAGAGCGGTAGATAACCCAAATACAGTTCTTCTCTATGTCTACGCCCA |
| | | TTATGGGACGCGTGGAGTCTCTGTGACGTTGACGGTTTATAGGTTAAGTATGCTTACGGAT |
| | | GAATATTAATGAATCGTCGTAGTTATTGAAGACGGCCGATGTAGTATGCACCGTCAGCCGA |
| | | TTCCAAACTAGTATCTTGCTCCTGAGTTACTCTGTTAGATTCCTGTCAGTTTATCCATTTT |
| | | AGTGTAGAAATATCCTTGAATGGTTGTACCATGGCTCCTAGAACTAGACAAGATAAAATGT |
| | | TATACCGTCTGGTGAACATTTAACCTCGTACTTATCCGGACTAATGGTAATTGTCGACCGC |
| | | CTCCTGAAAACTCGCATTGGTGTCGAAAAAAGCAATGAGCGCGTATTTTTATGGAGATAGG |
| | | TGCATGTATTAGTCTGTATTCTTAGATGCTCTGTCGATAACATGATGTAATGCGAATTGAT |
| | | TAGAACAATCTGAGAGGCTGAAATTGATTGCCTGCCCAAACACGATACGGTTCGATAGCTA |
| | | GCTGCCGATGCGCTTCGATATTAAACGTAGGCAAAGACTTCCATTCTGTTGGTGGTAATCC |
| | | TATCGATTCCTTAATGAACCCACGACATTGGATATTGATATCGTGCTTAGATATTTGCCAC |
| | | CATATGATGTATATAATTATAATACATATGCTTAAGGCGATAGTATTTACTCCCTGTACGC |
| | | GCAGTTAGCGTTGGCATGTAACAATTTAATGGCCCAATGAAGCGACTACGAACCATATAAT |
| | | TTGCTACAATAGTACTATTAACATGCTATGAATTTATGCAAAAAAAAA |
| 334 | 38.80% | GAGTTGATTTTCCGCATTTCATGGAAATATAATAGGGTAACGTTTAGTTACGGASCGTATT |
| | | CTTTTGAAAACTCTACTTAGTGTCGCAACTAAACTTCTCTGTTTTAGTACAGTCAGGATTA |
| | | GAGACTACTAAGAAATTCCTGATCTGCTCGCTACTGCCACACTTTACGCAGGAGGCTTGTT |
| | | TTCGCAGTAACCGGTGAGTTAAGGTCCAACAGGGTCAGATGTCCCTTTTGTCACCACGAAT |
| | | CACTGGCTCATTAGAAATTGATAGATTTGTTAAAACGAACCTCTATGTCAACAAATGCTTG |
| | | GAACGTCATTATGACAGTGTTTTGATGTCAGTTTATCCAGAAGGGCGAGAGGGTCATGGCG |
| | | CGGTCAATTAGAGGTTCGCATATTAGTACTTAGGTATTGTCAGATCACCGGAGTTTGGAAA |
| | | CCCTGCTTGTGTGATACCTACAACTTAACTTGGCCCAACATGAGAACGTTCCATGCTTCTG |
| | | GTATCCGTGTTTAAGCTCTCAGTGGAGAAATTCTTATAATGATATTCGTAACTAAAGGCAT |
| | | GAAACAASATGTGAGGATCGGTTATAATGGACACAGTCCTGACCCCTTCGATTGACCTAAA |
| | | ATATTGAAACTACATTCAAGTAGCGAGAATTTTTTAATTGTTCCTAAAGTTTTATTATTAG |
| | | ATAAGTGGTCGATGTGTAGGAAATAAGAGATGATAAGAAAACCAGACGTTATTTAAAGGGA |
| | | AATGTCCACCAGTGCCCCAGCGTTATAACATGATAGCCAAGAATTTGGTTATACGCAAAGT |
| | | TCGATTGCGTGCTCGGTTACTGGAGATCAAATTAATGGAGCTTCAATAATAGTACTAAATC |
| | | ATGTTTTCAATTTCTTAGCACATCCCCACTAATAGTTTGTCTCAGATATTATATGATATAG |
| | | TTGATCGACCCTGTTATACGCCTAAAACCAATTCTCTTTCGCTACCCGAGAGTGAAAACAT |
| | | ATTCAAAGTTGTCAGCCTCGACGTTTAATCTTCGTAATAATTTGTCGGTAACAGATTAAAT |
| | | ACGGAAGACAAATATTATTATCTTCAACTGTCCAAATTCTCCGTCTCCATTTGAGACTTAC |
| | | TCATACTTCAGTGACCTTGGCACTATAGCTGATGTTTGGAGAGAATTAAACCGAGATACTT |
| | | ATAATAATGAGAGCTAATGAAATGGTAGTTCGTATATGCGGTTATAGACTGTAAGAACTAT |
| | | CCAACAGACTCTGCCGCACTCTCAGATTTCATCTTAGGCTAGGTTATAATGTATGGGACGG |
| | | CTCGGATATTCTATTGAATTTAACAATTTCGTCCAACAACCCTTGGTAACTGAGTTTCCCG |
| | | ATTACATGACGATCCAGCTTACCGTAACCATAGAACTTGGCAATCCTCTCCTTAAGGCGCA |
| | | TGACTAGATCATCAATCGCACTTCTTCAATCAAGTTCTCTATCTGGCGCGGACATACTGTT |
| | | TTACGTCTCGTTTCATTGTAAAAACCCTTCTGTGTAATAAGAACACGCGACTTTGATGGTT |
| | | GCGATCCCTACGTAACGTGCACTTAACTACATATACTTGGTGAGATTGTGCTCCATATTGA |
| | | AAGTCGATGTTAATCAAGACGGAGTTGTGATTAATAAAATGGCATAATACACCTGTGTTTT |
| | | TCCTATATAATCCAGAGAGGAAAATAACTGTTTTCCGACCAAGTTTGTACTAGATTTATGA |
| | | TTTTCCGAATATGCATCTGCGTGAGTGTGTACGTCTGTGTGCATACGTCATTCAGAAAGAT |
| | | CTTCCGTATGTGAGACCTTTTGGATCAGTTGTTCATTTTTGTACCTGCCTACTTTAGACCA |
| | | GGTTCTAAAAGGCTCATTTAACACATGATTATTATAGATCATATAACCATTACTCGTAATC |
| | | TAATTTGTGCCATCGTTGCAACCGAAATCGTCTAGCAAGATGATCATCGAGCAATACCGAC |
| | | CCTTTATATAGGCTCAAGCCTATATTCAGAGGAAAATCACGGTTTGTC |
| 335 | 38.90% | GTCCATCATTGACTCTGTTTTCTCGAGGAACTCTGCAAACCAGATAAGAGATTATTAGCAT |
| | | ATATGTACCTAGAAGGACATATTATCGTGGACATCCCGGGTGTTTGCTATTTGAGATTTAT |
| | | TGATTGTTTTTTGGTAAAAGATCTGATTTACATGGCATTATAGCCGAGGCTCATGTTTACA |
| | | TTAGCATAGTAGGCTGGACTAGTTGCGAGAGATTTTGTTACCCGGGATCAATTGCCATTAC |
| | | ATCAAATCACGTGAAACGCTTTTCCAATACATGCATATCCCAGCCGATACTTAGTACGAGA |
| | | TGATAGTTGTACGACGGATATATAATTACGTCTATACGTTATAAATTGTCACCTGTCACCA |
| | | CTTTCTGAATTAAAAGCTGAGGGACGAGCCGTATTAATACTAAGAGCGTAAGAGCCTCCTA |
| | | GGGTTATATAACTTCCGCACTCAGCTATTATTATTGAACCTGCGTACAAGTATCTACTTAT |
| | | TCAAGTTACTACGTATGAATTAGTAAGCATCTTGTTTTACTTATGACCGCAATTTCATACG |
| | | TTGCATGATAAGACAAGTTCAAGCACAATAACTACGGCAGTAGGAATTGTGGCTCGACAAG |
| | | AGAGAGCTGTTTTCGCCGTTCTGGGGATGAGCATATTTAAAGTTGTTTAACACATCCTTTA |
| | | ACGATAACAAAAGACATACACAGGATGAGGTATTTCTGTCAAGAGAATTGGTAGTTTGTGT |
| | | TAAGAAGATCCCTGACCGTCCTTAGATGGAAGAATTAACGTCCATAGCTGGAGGTGTTGTC |
| | | TTTATTCACGGAAGCATAAGAGACTCGTAGTACAGAATAAGACGGTCTCAGGGTATCCACC |
| | | AGGATCAACGCCAGAAAGTGGGCAACAGATCGGAAGTGGAATTCGGAACAAACTTCATATG |
| | | TGAAAGAAAAGCTTTGATACGACTTCCATGCCTTGGTGATAGGTCAAATTTAGCTATTAGA |
| | | AACTGCAATGGGAGATGTTCGTGCATGGGAAGTAAATGTATCGACCATAATCGCTCTGCGG |
| | | GCTAGAGCTTGCGGACAGTTAGCGGTTCTTTAGACGGGCTGAACCCTATCGAGAACCGATA |
| | | CAGCAATGTAGTCCATTACGACATATGTGCTTCCTCGACTTTACTGGAGAACCTTAAGACG |
| | | CGATGGATTATTTAACTAAATTTCCAGTTATCTGAACTGGCATAATTTACAACAAACCTAA |
| | | ACATTTTCCATAGAAACTCGTTATGAGCATTTCATGCAGTGCGTCCACTGTGATATCTGTA |
| | | ATGGTAATCGGTCCTCATGCGATACGGCTCGGTAGTTTGTCTTGCGACTTAAGGCAATGAT |
| | | GTGTGGCATGCTGTCCAGAAGCAGATAGATCAGGGTCAAGTATTGCCCGCCCATTTAATTA |
| | | CTAAAGAGAATAATGCACATAATAATCTCTATTGTTAATGATATAATTATTCTAGTGATTT |
| | | ATATCTTTATAAGGTAAGCGATTTCAACAAATTAAATTAAACGCCATAAATTTCTAGCAAT |
| | | TTAGATACTGTATGGGACTATTAGGGACTCCATAATTAACGTATGACATACTACACTAATA |
| | | ACTTAACTCTATTTGACAGTTGCATTGCTTAAACACCCTTGTGTGTTAAACCATACAACCT |
| | | TATGTCTGGCTATATTTGTACTTCAGGACCGGGATTCATGATAAGTGCTTAGGAACCTAGA |
| | | CGATGAATCAAGATCAACGTCTTATTTATAAAACGTTGACACAATATTAATCCTACAAGAT |
| | | CTAACTTTACCATTAAACAGAACTTGCTAATCCCTAATGACCAACAGACTTCTGGCAACGA |
| | | GAAAAAAATAATCATAATTTGTGCGGTACACTTTAGCATTAATTTCTAGGATTCAGCTAGC |
| | | TGGGCCTAGGGAACACGAGCTTTACGTGGCGTCGTCCGAATCGTTAGAGAAACATTGTGAG |
| | | ATACTCGATATTTTTATCGGTAGAATCCTCCCTCATTCTTACAATGTA |
| 336 | 38.70% | CTCAACAGCATTCTATAGCCACTAATCTTATCTCACAGGCGCATTGCTGCCATACCGTTAG |
| | | AGGGTTTATGAGTGTGGTGCCAAATTTAATTTCCAGCTATTGCTGAGAAGTCATATAAGTT |
| | | TAAGTGCCTCTATTCATGAATCTACGAAGACTACGCCGTCTGCGCACTGGCTTTGCCGTCC |
| | | CACTTAATTTAACGTTAATATGCAGGTCCGGGTTAATTCATGAAATTTATACGAGGGGGTA |
| | | GATTGTCGCATTATACGCTCACCTACAAATCTGCCTATCAGCACAGCCATTATGACTAGAT |
| | | TTACCGGGGAATTTTCATATACACAAACCACACTCATTTTCCCACTTATAGGATTGAGTCT |
| | | CAGATCACACTTGTGCTGCTTGCTGCAAATCCTTTTATCATTGTTCATGGTTACTTGTTTA |
| | | ACTAATATCATTCATTTAAGATAGGGTATCTTTATACCTTGAGGCCAAGTTTTTTCACAGA |
| | | ATACTGAAGATCGAAACCTTTACTTCAAATAGATCAGGTAAGATTGTTTTTCATTTAAAGC |
| | | GATTCGCTCATACAGCTTTCTGTTAATAGTGATATGGATTGGAAACTAAATTACCGAGATA |
| | | TATCGTCATCGTCGGCAAGCAGCTGCTTTATACTAGGATACAGAAGACGGCCGTTTCCAGT |
| | | AAAAAAACCGCCGATTCGATCTTCGATTATTACCTTTTTACTTGCGGCACCAAATGTAGCT |
| | | GAATTATGTTATGAGCTATGCGTAGTATACCCCCTTTGTCCTAGTGCTAGGCTCTATGATT |
| | | TTATGAAATTTAACTCTTGCTCCAGGATACGTCGGATGTACTTTTAACAAAATCTACTGAG |
| | | AGGACAGGATTGACCACGTAATAGTAGAACTGATAGGCGGGATGATAGGATCATGGGCAGT |
| | | ATTGCTGATTTTAGACCTTGGAGATAGCTGCTTAATGAGCTCCTCGACCTCACACTTACTG |
| | | CAAGGTCAAGATAAGAAAATCTCCTAAAGATCAAACCATTCCAAATTCGTGTTTACATAAA |
| | | TTTTACTATTATACATCGTAATGTTAAGTGATTTAGCTACTGTGTGTCTAGGATCCAGGAT |
| | | AGTCGTCTAAGAAGCCGACCAACGTGCTAAATAGGATTTGAACAGCGTTATAGTTTAGTTT |
| | | ATAAGGTTGTCTATTTTATCAGTTACTGCACGACACATATACTCTCAGAGAATAGGGTATC |
| | | ACGGTATACATCGCTATCATATTGACTAACGATTGTTCACGGCTTATATTTTCACGAGCAT |
| | | TCCAATGTGGTAACCATTCGCAATGATCTGGGCTCTCAGTTGTTAATGTAGAATTTAACCA |
| | | GGTTCCGTATTAGTCGAAATCGATGCTCTATGACCTCAACCTTCCTCTTGTCATGATAGGG |
| | | TGACTAAAGAAGTTTCCGATACGCGACGTGAAGTCCGATTATTATCCAGATGGTAAAGTGA |
| | | AGCTTAAAACATAAGAGATCATTCTCTCTGATGAGACATAATGATATCATTTCAAAGTTCT |
| | | GTTAATAATAGAACTGCTAGTCAACGGAATCCTTTCCATCTAAAGGCGAACACTAACTAAT |
| | | TTGAATGAGAAAGATAACACTAAAACCGCCAACCTAGTAGTTACTTGAGCTAACACATATA |
| | | TTACTTAAGTAGCTTTATCTCTGGTCTAAGTCGGAGGTCACAATGACTTGGACTTCTTTTA |
| | | GTTTTTCGAGTACAACTAGACAATGACCTCCCGACGTAGCATATAGAAAGTTAGAACATAG |
| | | GATTACCGAGTGGTAATAGCCCAATCAAATTATGGTGCGAAAAGATAGTACTGTACTCATT |
| | | ACTTCCGGTATGGGACAAAGCCGATCTATTTGTCGGAGCACGTTAATTTTATGACCGGCTA |
| | | CCCTACGTTTACTGAGTCTAAAAATTTGTAAATACAAAAATTTTTCCCGCGCTAAGTTAAC |
| | | CATAACTCTCAAGTTATACGGGGTAATGGATCTTAAGTTCCCGGAAAA |
| 337 | 39.70% | GTAAGACTGATTAAGAAATTACATAGGGACCTGGAACCGGTATCAGATTTCAAATTTTGGA |
| | | TAATAAACCGCCAGGTGTTAACCCATCAACATCTAGTATTGGCGTAGTGAGATCTCTTGCA |
| | | TTTCAGACATCCTGGGACGGCAGGAGTTTCTATCCATTTTCCGCAAGTGTTATGCTCCAAT |
| | | TGACAGATATGTCGCCGAGGAACACCAATCTGGAGAATATTTAGTCGAGAGGCACAACTGG |
| | | TGTTATAATCTTAGTGTTATCAAGATGACCTTTTGGAGTCCTTTGGATACATGAACCCATA |
| | | CAAATTATCAGCGCTCTACTCTTCTGTAACACCTCGGAAATACACTGAAACAGATGTCAGA |
| | | GATAACCATGAGTGGTGATTGCAATCGGTGACCATGTTCGTAGATCAGTCCTACGAGCGTC |
| | | CATATGGCGACGAGGGAACTCCACCTTTCGAGCAATCATATTGGATTGAGCAAATGGTCAT |
| | | TCAAAAATATACTGTTCACTCTGCCAATATAAAAATAGCACTCGTTTTTTCTATTAGGACG |
| | | ATACTAAGTGGGCACTTTATCCCTAAATAACTTTCACAAACCCGATTATAGATCCCCCGTA |
| | | TCCAACTGGTAGAAGGCGGCTCGGATCTATCAAGCATTTGCCGAATTTTGCGTGAAATTTT |
| | | TCCACTGACTGCTAAGCATAAACCGATGAAGCCAATCTTGAATGGGTTATCTTGAAAATAT |
| | | TTTGCTAGATTTCATAGAAACTTTGATTAACTATATACGATATACTTATGAATAACGCGAA |
| | | TTACATATATAGACATGTTCTACGTTCCCTGACCTTGCGTCAACAAAAATCGGTTATGTCT |
| | | TAATCAGAATTGTATTATAATACATACGTAGCCGTTTTTTAACTACTGCTTATAAGAGAAT |
| | | ATTTCTATACTTACTACACAGATGTTTGGACTATAAATAGAATGACATGGGGGCAGGGGAA |
| | | TATGTATAAATGCCTGTGTGATCTCCAACTGCGCATTTTGCCGATGATATGTAGATAATAC |
| | | TTTGAGTCTTGGACGGCCAACGCGGACAGACTACACACTACTATAGACAATGGATGATTTC |
| | | AGACGGAATAAAATGCTAAAATCCTACCGATTGTCATATTTTTAAGTCTATACCTCACCGT |
| | | ATATTGAATTCATGTCGTATCCGAGCGATTTTCGATTTGCCCTGAGACCATAGATAAAACT |
| | | CACTGAGCTCTAACGTAAGATTCAATTCAATCAATTATAAGAGCAAAAGTGTAACCCGTCG |
| | | AAGTTATTAAGCTGAAATAGTCGCAAAAACTGTCAGGTATTGCTGTCCAAGTTAGCGGGGC |
| | | GCCATGAGAATGTGAATGACACGGCTCCTTGATATCACAGCGTCAATGTTTAGGTGGATTA |
| | | GAGCAGAGATATAACGAATGCTCATCCGATATGACGTATAAACAAATGAGTAATGTTAACA |
| | | CTTTTATACTCCGGTACCTCAGTATTCCAGATCTGACGTCCGTGGACACAGTCCTCAATTA |
| | | CGCTGTTATTGTATGGACTACCCATCGCTGCTTGACACGATCTTGAATTTATATAGCTACG |
| | | AATGCAGAGGTTTTGCACCGCTTGGCACTACCGAGTATAAGGATTATGTCAGTCGAGGCCT |
| | | GAAGCGGGGACTGTGAAAAGCACTCCACACACAACAGCCAATGTAGAGCCTTCGTGTTTGA |
| | | AATTCTAGGTTTTCAACATAGTTTTTTGGCTGCTATTCTATTAACTACTAGCTTTACTTGT |
| | | AATCTTCGGCTAAAGTAGGAATGTATTAATTCGCTCACCGAATATCGCCGATCCTTGACCA |
| | | CGATGTCCCGTCAATTTGTAAAAGGCATCTAGTATTCATCACGGTATGGTATCCCTTAAGT |
| | | TGTGTATGGCTACAAAAAAGTAATGGAATCTAACTAATTCCATCATGCGCGATTCATGAGC |
| | | TCGTGTCTGTATGAAAGAATATACCATTCAATAGACACAACAATGATT |
| 338 | 39.50% | CAAGCTAGTCTAAACTAACAACAGCAGGAGGGCGAGAACGTTGGCCACAAGACATTAGGCG |
| | | TTCTGTTTATCAAGCATCGACGTCTAATAATTTTAATACTAAAATTCGTCACTATCTAGTT |
| | | GTTCACCATGGATTTTTATGTAGGCGATATCAATTCAGTAAGGTAACCCTAGTTCTCTGGG |
| | | CTCATGTATGAAATCGGGAAGAAAGATATGAATGAAAAGAACCTAACTACTGAAGGGTAGT |
| | | CGACGAGAGGCAGCTAATAGGCAACCTTTGTCCCTTCGGACGGACTGGTTGCTGAAATTAA |
| | | TTTACATAAATTAATGAAACATCCCCAACGCCACCTTACCCATAGGGCGTCTCACGCTATA |
| | | CGGTCTATTTTAATGCCTAAGAATTTACGATGAGCCTATAAATACCTTAGTTGTGAACGAA |
| | | ACGCAGCACACGACAATCGTACAACCTCACTTTTAATGTTATATACGGGCGCGGCTTGGTA |
| | | AATGCCGTAGCTCTAGTAACATAATGCATCCTCACCATACCAGCAAAGCTAAAAATCTTCA |
| | | AATATTCGTATAAAACTAACCAGTTTAACGTGTATGAGGCGGTCTTTTTACCAGTTTGGGA |
| | | GCATATTGCACGTACTATCTTCTTTTTAGCAGACCTGGGATCTGAGAACTTCCCCTGGGTA |
| | | GTCTTACGATTATAGTTAGCCTAATAGATTATTTGTTCGTTAGGAAGAATTCATATATACT |
| | | AGGTTATCCTTCAGGTTGAAAATTAAGGACGTTACAGATTTTTCACAATTATACCGACTAC |
| | | CATAAGTGGGAGCGCGAATAGCATTTGAGTATTTGGATCAAGCATCTGCTGGGTTACACGT |
| | | ATTAATTAGACCCTTGCCGAGATCTAGGGAAACAAAATCCAGACCCGCAGTACGTGGGTGG |
| | | TATGACGCTTCTTAGGATAGGAGCGCAAGTCCATAGACCTTTATATTACTACGTTTACCTG |
| | | ATCTAAATAATCTGATAGAAAATTAACCAGGAGTCCCATTAAGGTATTCAACCACGGAACA |
| | | GAGTATAATCTGGTTGATAAAGTCGTTTTGATCTGTTAAAGATTTGTTAAACTAAACGAGA |
| | | CTTCTTTGGGTAACATCATACAAGTCTGATAAAGGATGATGCAGGGACTAGTCTAAAATGA |
| | | GGGAGTCTTTGGGTATCCACCAAATAATTTCAGGAGTTAAGAGCACTTCCAACGATGCAGT |
| | | CCTTTGGCCTTCTCGTGCGACAAGGCAAGAAAAGTTTATAACTCTACAGCTTGTGTAACTC |
| | | GAAAGCTGACCTACTATATAATGTTATTGGAAATCAAACTCAGGGTTATCTTCAAACAGTT |
| | | TGTTATTGGCTAGACAGCTATTACCTTTAATTGGTCCTTAATCTTGCCTATGGACATGCTC |
| | | CACACATTAAACATACTTAATGGCATGCAATTATAGATTGTCCCGTTCATTCACTATAGCT |
| | | TCATAATGGTTGGGGTAGTACACGCAAAGTCTACTTATATGGGCAACGCGCCGGCCCGTCT |
| | | TTCCTGTTAAGTTACGGGAGGTCGCTAATTACTATTTTACTGGGAATGCGCAATCAAATCT |
| | | TGATTGAGACCAACGCCAGGCCCGAACTATTCTTATTGTTCCAGAGTCTTTACTTGAATGC |
| | | ATAGTATCGGGATGGGGTGATGCCGGCCACCGGATCACCATGGATATACGTCAGTTGGCCC |
| | | ACGTGTTAATTAATGTCATATTGTTATGGGCTAATACATTACTGTATTGTTTAAATACAAT |
| | | TCGTCATGCATTATCAGTACTGTGTAATTTATATAAGCGTTCATCATTGAACGTGTATTTT |
| | | GTTGGTGCGTACTGAGTTAGATATTGGAGAAATTCCCTAACCAAGGAACAATGACTGGACT |
| | | TGTTAGCGATGTAAGAGTAATGCAAAAGTTAATGAGACTGATATTGGAAACAGTATTGTTT |
| | | AGGCTAGTCTAGAAATAAACTGCTGATAAAGAATCTTGCAGTTAATAT |
| 339 | 39.60% | TTCACTATTAAGTACACCTAGTCAGACGTGAAAGTTAGTTCTTTTCACGTCTCATATAGTG |
| | | CTATTTTCGACCACGTCTTGCAATCGTGATAGACAGAGCTGTCATTAACAAGATCAAGTTA |
| | | TAAAATTGTACGGGTTGTACCTGCTTATAGTTATATGTTGAAATTGCAAGGCCGCGTTGTG |
| | | ACCGGTTTGACGGAATCTGAAGGGATTAGAGGAGTTTATATTTAATTTCTTTCATGTAGAG |
| | | ATAGAACCGAATAACCTCTCGCTACATAGAACTAACGTTTTCGCAGTGATTTACCTTGTGA |
| | | AGTGCACAGTACACTTCACTGCCTTTTACTCGCATATTGATACAGTAGCGAAAAGTATCAT |
| | | TATTAGTGCATAACCTTCACCTATTCCAACGGTTTTACGCATTCTGCGTACGTTCGATTGA |
| | | AATAGAACAAATATAACTATAATTGGTACCCATGATGTAACATTTTACCTCAGTAATATGT |
| | | CGAAGATAGGCTAAGTCCCCAGCTAGCGTAACTAGCTAAGCCTTGATGCGTATTCCTTAAT |
| | | CTTGTTTAACGTCTCTGCTTACGCTAGTTTTTAGTAGAGCATAAGATAGCAATTTCAGGAT |
| | | GGAACGAGTTATAGAACAGACCACTCCTACAGTGAGTAGGGTCACATGTATTGTCCGACAC |
| | | TGTTTATTCAATTCCAATCTTTTAAGTGCGAATATAATAAGAAGCACCCTTTCAAACAATT |
| | | GTTATAATACGTTTTCATGACACCAACGATGTCGACTATGATGTGCTTCTCTTTTGGTTAG |
| | | ACATCTTTGCATTTCGACGACTCCTTTTCATTGAGCAGGTTTTAGTTAGCTAAGTGTTTCC |
| | | TACATTGTAGCGCATTAGTCTAATAGAGAGTGAGCATTAGTCACAATATAGTCCAATGGAT |
| | | CTGAGAAGCCTTATGAGGCGTGCTTAGGGAACAATTGCAGTTTAGGCAGAAAGAGTTACCC |
| | | TTTAAGGGTGGTATTCTTATCTCATATCTATCTTATTGGTGCAAAGTTTGTCTTTGAACGA |
| | | CAGAGTAACTCCATTCGCAGCCTTGCTAAAAGTGGAGAGACGCAAAAGTGGAGGCACAGGT |
| | | CGTTTCTTTTAGTCGTATATCCAGTTTATGAGCTTCACATTTAAGATCAAATCCCTTCTCG |
| | | AAATAAAAAGGATTCCCACTTTAAATAGGCGATTGATTGTGCGCACTATTTATTCGTAATC |
| | | TATACGTAAAGAAACTGAACGCCACAGCCTAATACATGCTAGTATTTCATACATGTGAGCC |
| | | GAAGACACGCACTTCCTTTTTGATGCGAGAATTTAGGGCGACCAAGTCTGGTAACATTCTG |
| | | TCCTAGTTGCCGAGTAACATAGATATAAGCCTTAGCAGGGCGCGGCTATACCTTGGTAGTA |
| | | AGACGGGTGTTTGAGTAATATTAGTAGCTTAATTAACAGCGGTCAATCGCGAAACGGAATT |
| | | GTAACTGGAATGTCGTATAATCCCATTTATATCTCAGCACATAAATCAAAATGGCTGTGAG |
| | | ATTTAAAGAGGTTAGTAATTGTTCAGAAATCCGAAATCCTCATTACGAAATAAAATTCGCA |
| | | TATGCATACTTGATCGGCGGAGCGATGAAAGAATTACACTTTTAGTATCCAATTATAAACA |
| | | TCATTTGCGGCCTACTTTTCCCAGTAAATCAATACGTGGAGAACTGGCTCGTACTCTGCTC |
| | | TACACTTATTGAATGAGTTAGCCAATGTAGAGCTGGATACTAAGCTCTAGAAGTTACTCCA |
| | | GAACAATTACCACGTTAATAACTTCTATTATTCAGAGTCGTAACAGCCCTCAAGTCCTCTC |
| | | TTGTTCGCCTGTCAGCAATCTCCTACGGACCTACCCTGCCAGGTAGTTGCTGTCTAAGCCA |
| | | CTATTAGAGTTGCTAGATTTGTTAATTATAATGCTTCGCCATAGTCATCCACGGTCAGGGC |
| | | GGTACCTCGCAGCTTGTGTAAGGGATCCCTCGAGTAACTCTTGATGAT |
| 340 | 39.60% | CGTAGTATTTTGTGAGCTAGATGGAGTACTCCGATTCAAGGTATTATGAACGATAGATACC |
| | | GTGGCTATATCATAGGATTGCTACACTGTAGGTTCCAGACCTTAGCGAAGCGGATACCTTC |
| | | CGTTCGGTTATCTGTTAAAAACTTTACATCTTCATGATAAAGTGTGCCTACCTTTGTATCA |
| | | CTGATGTACTTCCCTACAATAGATACTCTTTAAGACCTGAGTACGCCGAAAGAATCTGTTC |
| | | GATCTAGCAACGACAAAACAGTTATCAGCATATCCGTATATTGTGGTGTAGCGTCTTCGTG |
| | | TACTAATTTAGATTTCTGCATCTGTCTAGTTACGTGTAGGGCCTATGACGGTCCCTTGCTT |
| | | TTCCCGGGAAATATCAATTGCAGTTGTGAAAATTGTTTATAGGAAAACACAAATCTAAATA |
| | | AATTACTCCAAGGATCTTCTCCCAGATGACTATTCTTAGATAATGAGAAAGGGAGACTCGA |
| | | TTAAGTAATATTGTCGAGCACCACAATCTGCCTATATTCTAACTTAGTAATAATTAATTAA |
| | | TTATGAGTCAACCAAAGGGTCGTTTAGCTGATTCATATACATACTATATTTGATCACCACC |
| | | TACGAGCAGTTGGCATAATTTCCTTGTTGACTAGTTTTGACCCACGTGATTCCCCTAAATT |
| | | TTTTGTGCTCTATGACCGACAACCACAGTGTAATGTCTCAGGTAAAAATGAGTACATACTA |
| | | CTTTTCCAGATTGCATAAGTTATAGACTTCGGTATTTTCCAAATATTATTGCATTGTACTA |
| | | CAAAACTAACGGGTATGAGTAGACACAAACGATCACGGGTTTCACTTATGAATAACGTTGT |
| | | AACGATAAGTGCGCCTCGCCTGCACCGCATCACTAACGCCTTTTTCGAGGTAATACCACGT |
| | | TCCGAAGAATCTATTTAGTTCCTCGAATAAAACATTATTGATAAGTAGTGAATCACCAGCC |
| | | TCCCAAAAATACCAGAAGAGAGAAACAGGTCTTTCAATTGCTGGTACTATTTGATATCCTT |
| | | TACACGTTTTCTATTCTCCAGTGTAAGTCTCGTTATGCAAGTTTGTCAATATCAGAACAAT |
| | | ATGATATACAACACCTCGCAAGCTGCTAGCAGTTAGATGCGATCCGATGATGATCGATAAA |
| | | AACTTATGTACTGGACCTGCTGGTTTAGCCTTTAAGAATAAGTTGATTCTTGACATACAGC |
| | | TCGGGCGATAGGATTGAAGAGTAAAAGCGATGTAAACCAGGTCTGTGTTCGATGCAGAGCA |
| | | AGTTCCTGCATCGGATTTTTCGGATATGCAGCTTAGATGGTTACTCAAATCCAATTCCGGG |
| | | CTGTTGTCTGTACAATTTGGGAGGTTGACATTGCCACCTGGGCAAATGTTGTCCGAGAATT |
| | | CGCCCGATGAGAGAAGGGACTTGGTGGAGTCACAAGAATAGGCGATTTCGCCCCAAATTTA |
| | | ATATCCAAAAGAAGGCGTTCTACTAACCGTAACGTTAGACATATTCGTACAGTGAAGTTCG |
| | | CACTATGTGTGCATTACTCAAGTATCTGTTGTATAGGATACCTTAGTGGTTCAGTATTAAA |
| | | CACGATTCTTTTATCTTGTATGTTGTAATAGCGATCGTTACTTATCAACAGAGTTAAACCA |
| | | TGGTACAAGTGCACAAGTCATTAAGCATCTAGACTGCACTACATCGCTTCTATATTCACCA |
| | | TATGACGTTACAATCTCCCAAAGTAAGTATGTGACAACTTCTCCGGCCAGCTACATCCGGT |
| | | AGAATTGTGTTAACTAACAGTGTAATTATACTCCATCATACGATTTAACCGGTTGAATGAC |
| | | TAAAACTTAAGTAGTTCTCGCATGGGTCTCCGCCTCACTGGTAATATGTGACCGCTCTATT |
| | | GAATTCGAGACCAGGATCAATTACATCCTCACCGGGTAAAGAGTAGATCAGGATTTTTAAG |
| | | TGAGTAACCTGGCGATGAATACAAGGTTGTACTGCAGTTTTACCCTGA |
| 341 | 39.20% | GATTTAAATGGTAATTAAAATCGAAGGTTTTAAAAGGTGAGAATTTTTTTATAAAATGCAA |
| | | TCTGTTACGCCCCTAATATTCGGTTTCATGATTTGCTTAATATTGTATCAAGACAAGCATA |
| | | TTGTTAAACAGTCTCTGTACTTTCTTGATGACCAATAATGAACAGATGAAGTCTTCATATA |
| | | TTGAACTTCAATTGAATGCGTGCATGCCATTATTCGTCATCGAGAATTAGGAAGAAAACAA |
| | | TTGCAGCCTTCTAGCGCCAATTGCGATTAGTAAGCTTCGCCCTGACGTACTAAATTATATT |
| | | AGACTGATCGGAGACATTAACAAGCTGCTTATTCCGTCTTGAAGACCGTATTTCTTACTGT |
| | | TACGGTGTCCTTAGGCGTCATATATCAACTAATATAAACCGGTACTTTATTCATAATAGCC |
| | | GATATTCAGTGATTGTTTGCCATAGGCTACTTTCTTTCCCAAATCCCCGGTATCGCTATCC |
| | | TATGATTTCTGCGTCAGGGGTTAATTACGGCGACACCAGCCTAACCCAAGATCAGACTAGG |
| | | ATAATATTTCACTGGCAATACTCATCGATTAATTCAACTAGTATCTATTTTTTCACACTCC |
| | | GCAAAAAAGGGCAAAACAAAGTCGTCAAGCCGGGAATAAGGGTTATTCTTGCAGTCTTCGT |
| | | AATAAAATTTGAACTCAGTTATTGCGAATTTACTCGTATAAAGCTTCTATTATCATTCTCT |
| | | GATTACTCAAAAACGCTCCATGAGGGTAGTAGCACATAAGTAGAATTGCTCATAGTGGCTT |
| | | CTTTCTCTCAATCCCTTTGATACTGATTTTTATATTACTTACATGTAACGATTGTTGAAGG |
| | | CCAGCAAACCATATAAGTGGACAGAACAGGGAACAAGAGAAAATAATACAGAAAGTAGTAA |
| | | CTAGTCAAGAAAGTCTAGATGAATCTATAAGTTGTACCTATCGAACTATGATCGTAGCATT |
| | | TTCAGTCTACTTGAGGGAGAGGCTGTAAGGAATTTTAGCGGCCAGATATATATCGCTGGAA |
| | | CCAAGTTATCGGATGGAAACTTGATCACGTACAGAATGTGATGTACGCGCAAATTAGATCT |
| | | GAAATCCCTCTGTCCTCATTTTTTAATTAATACAATTAATATCAAAGGCCTTCTTTTCTGA |
| | | ATGTTATTAGACGGAACACGGAACTGCGATTCATCATCCTAACTACACAACACGAACTGAC |
| | | CAGATTTGCGTGTAATCGTCACGTGCCGTTGCTTACTCTAGTAAACCCCGGCGCAAGGGCG |
| | | AATTGTGAAAAAATGAGTCAATTCGCTACAGTGGCAAAAAACGAGCTCCTGGACGACACAA |
| | | CCTCGTATAGCAAGGCGTAGCTCAATGCGCCAGATATTCAGGTATTGTAGCCCATGACAAC |
| | | AAGAAATAAAGCTATAGTAGGCATCATTATCGTTTCGTCCGGCAGCTTTTTTCTGACTTCC |
| | | ACCTCATTGCGTCTTATGTCATTACTGCGTAGGGTCACCTATATGAGTCTTCATCCCTGGG |
| | | ACACTGAAGGGAGTACGCCAGTATTTCATCTATGAATAAACCTCGATTACTCCTTTATGAG |
| | | AACAATACTTACACTCGACGGGGTCTTGTGGTAGTGATCTTAAGATTATCTACCATTTGTT |
| | | CACCCTTGAAAAAAGAGACTTACCTCTCGACTTTTTTCTATACTGGGCCCCGACCGCTGAC |
| | | ATGCAGAATATTGAGGAGATGCAGATTGATATTTACAAAAATTAAAGCAGATACTCAACGC |
| | | ATATTCTATGAAAATCAGGGACACCCAGGGTGGTGCTTTAGGATGATTTACATGAAACTTT |
| | | AAAAGGACCGGGATAAACTGGCCGCCGGTCTTTCACTGCCACAGGGATCTTATTCATTCGG |
| | | ATATATTATTGCCACTCAAGATAAATTCTGTTAGTAAGTGTTAAAGTGTATCATTATTGCC |
| | | CATTCTTCAGACTCGAGAACTTCGAAGGCAAATGCTGGACGTGTGTAC |
| 342 | 38.70% | AGATCCACGGCCCTGAAATCGCCATCGCTGTTCTTCTTTGATGAATAATGGAAGGGCTGAG |
| | | TTCATCAGTGTATTCGAATGCTACTATATTTCAGTATTGTGAGTATCACAGCTGTAATCTT |
| | | CGGAAATACAAGGATGTTTGTCGACCTCGCTAACACTAGATTATTTTGGCCCGTTACTATT |
| | | TATATTTTTATGACTTCAAAATGCGCTTCAAGATTGTAACTCTGGTTGATATAGGATGCAG |
| | | GGACCGGCTCAGGGCCGCTCTGCACTACATTAATACCTCAGGGATCTCTATTTCGTTAGAG |
| | | CACACGACTTAGTGACTAGAATAGCTTTAAATGTAAAACTTCATCATATATTCCTCCTGGC |
| | | TAAGCCTTAATTTCATTCTTGGGGCTGTTGCCAAGACTGCTCAAGAGTTAGTTTTTCTTTC |
| | | TCCTTGTAGTACCCGTTCTCCTAAGTGCAAATAATCTATACACACTTCATATTGGGTATAC |
| | | CATTCTTGGTTTATTGTCACCTGTTATGTATTTTGCATCAAAATAATCATCGATGTATACG |
| | | TTAACCCAGGAGACAATCGACCGGCTAATTCCGGGAACGTAGATGTATGTAAAGTAACATG |
| | | TATTTCAATTTCTTCTGAAGTATGAGATTTCAGTTGCACAAAAGGTACTCAGCATGTCTTA |
| | | TCATCCATAGGGCCGCAATTATAGAGGATCTTGAGTGGAGGGTCCATACGAGGCCTTAGGA |
| | | AGCCGGCTTATCTCAGCGAAGGTTATCGAGATGCTAAATTTACGGATAAAGATCCGTTACT |
| | | CTTCTTTAGAACTACCGTTCCAACTCGAACATAGAATCGGCTCCGAATTCTTGGGTACCTT |
| | | GCAGAACTGAAAAATAGATATCTCGGTATCTTAAGGCAGAAATAGTTTTCGCTCTGGATTG |
| | | GTTTCTAAAGTGAATCTGAAGTTCTAGGTAAGCATTCAAGTCCATTGGGGACCATTAGGGG |
| | | TTAATACGCACTGACGTCGGTCTTTCGATTGATAAATACTTAACCTCGTTAGCAGTGAGGG |
| | | TCAACAATCATTAATCTCCAGCTATAGAGCGGGTTAGCCAGATTTTATATCGGCGTCATTC |
| | | CTTTTATCTTTGAAATTTAGGCCAAAAAGAAGGGAACTGGTTCTATTCGCGAATTGAACCG |
| | | CATTTATGGTAATAGATCTGACCACGTGCTACTGCTCACTTACAATAGCTAGTTTTCGGCT |
| | | CAAACTTTGTATAAGGCTCACTAGGCATATAACGAGTTAAAACTTTTCACATGATACGTGA |
| | | CTAGCTTCGCCCGACATACTATATATAAGGTCTACCGTTGCGGGAAAAGATGAAGATGATA |
| | | TTATCAAGTCTTTGACTAATAAATTAACTTATGCTTACAAATTTCCAAAATAGATATTCCA |
| | | GTCGTCTATCCTTCTATTACAGAGAAAGGCAGACTTAATCCGTTCATTATATAATTTATTT |
| | | AGATGTTAGTCTTTCTGGTGGGTCGATTGTTAGTCTTTACATAGAACTCCTTTAATGTTCA |
| | | TAAGTTTCCATCAGTAGAAAGTGAGCTTATGGGTTATTCACCTTTGATATTAAAAGATTTA |
| | | CTACTGCTATAATCTACCTAGCTCAGCTGAGAGGCAAGAGGATCACATGTTATTGTTATAA |
| | | TGCTTTGATTGGTAAACTATAGTGTCAAGGCAATTCGAGTGTCGCCAAGTTACGTCGATTA |
| | | GATCGATCATTAAAATCTAATAATGTTTAGAGTTTGTTAGAGTAATGGTGTTGATCGGCAC |
| | | ATAAGAGTCAGAACGCGGGAGTATTGATATTTTGCCGAATTGGAAATTTATCAACATCGGT |
| | | TCTACGTATCGTTGATGTCCTAAGGCCTTAGTTACGTAGCTTACATTTAATGCGCATAGGG |
| | | TTGAAGCGTGTGTTAATCGCTCTTTGAAATAAGTGTTAGGAAATATACGAAGTAACGAATA |
| | | TCAGCCTAATTCCAGCGACTAAAATGAAACAAGAGCATCCGGTGGTAG |
| 343 | 39.50% | TTGATAGTGTGATTAATTAGCTGGTCATTATCGGTATCGTTGACAACAGTAGGATGATGGC |
| | | GATTGTCTGCAGATTTCGTCCATTAATATAAGTAATACTTGTTATGATGTCCAACTTAGAT |
| | | ATATTGGAGTTTTATTGCTCTATTTCCTGTACCCTTGTGACGAGTAACTGCTCCGTGATAT |
| | | AGGCAAGTTAAGTGTGTCGCAATATGGCAGTAGGCTGAATACCACACATACTGTCTTTCTA |
| | | AATAACACTAGGCGACTACCTTTAACTTCATCTAAGGACGTTATTTCACACTAAGCACTCC |
| | | GTCCCGAGAACAGGGTCTATTGAGGCTACTGATTGCGTAAAGTAGTTGGACACGCATGGGT |
| | | TCTAGATCCTCATCTCTGGTTTCTCAACATATTGAGTTATACTTTCTGTTAGTTGTTAAGC |
| | | CGGGCGATCAAAGCATTTCTACTTCAGAAATGGAGGACTGTAGTTATATACTACATTCTGA |
| | | AGCGGTACCATTAATGCTTTCCGCATTGATGAATATCTATATTTACAGTTTGGTGAACACA |
| | | ATTAGGAGAGTCGGACTGCGCAAACAGAATATTTAGTTACTTATAGTTAATATAGACCTAT |
| | | ACACGGTAGAAGGTCAGTTCATATAGACTTCTGGGTGTGTACTTCATCAGAAGTCTCCTGT |
| | | CTGTTTAGCCAATCGCCACCTTCTCAGTCCCGTGGGAGTACCACTCGAATAGATCGTTGTT |
| | | TTCGTTGTTGATAAACGGACCCCGTCTTATTTTCGTTACCATTTAATACGATATCATATAA |
| | | TTGAAATATTAGGAAACGGCATTTCAAATACGAACGATTTGAACTTCACCTACCTTTTGAC |
| | | GCAATCTGAAAAGTCAACATGGTATTTCTGCTTACACCGGTAGGGTTAATGGAAGTTCTGC |
| | | GCCCATTCGAATTTTAGAACTGAACAATAATTCATGAAAATTTACGTTAGCAGTACCTTTT |
| | | TGTCTTACTAGTTGTTGCAGAAATTTAAACATTACTTGGTAGCCTGCTGTGTATATAAAAG |
| | | AGCGATCTCCGATAAGTTGTTAATCTGTTGCTACCTAAGCGCTTACTGTGTGCCTTGGCTC |
| | | GCGTATATGCCCAGGTCAACATTTATTTGTCGCTCGACTCGAAATAATCTATATCATAAGA |
| | | TGGGAACGAGTATGCTCCATGAGGGAGCCGGACTAGGCATTCAATTTTGTTTGAGTCTTTA |
| | | GTAACCATACCTATTCATGCGTAGTTAACTTCGTAGTAAAGCAGCGTTTATACATAAACAC |
| | | CAAAAAATGTCCTAGGGGCATACCAAGAATCTAAGAAACAGCGCAGTAGTTCGTTCGGTTT |
| | | GGCAACCATACGAAAGTATCATTGCACACGACGCATACAGCATCCTAGGAGTTTACTATGT |
| | | CTTCGTTTTTTTGTAGGCCCCACACACATTAAATTCGATTTATTACACTCAGAGTACCTGT |
| | | CCGCCAATTCACGTGAGTACCTTCGCGCAGCAGATAATACATTGCTATGCGTTCAGACCAT |
| | | TGTAAGAAAACAGATCATGACTCTAGAAAAAGTGGCCTTAGATCAATAAATGTTAAATCCG |
| | | GTTCTCTCTAACCTCGCCGTACACAGTTAAAATCAACGCGCATACATAAACATTGATCTTA |
| | | TGGGGGCTCACATAGTGAGACAATAGTAGTACCCAGTGTTATACCTAATCTAATATATAGG |
| | | CTAAAAGGTAGATTAATTGTCTGATCATAGATCTCAACCGATCATGGATAGCTGGGAATAC |
| | | GTTATAAAGGTAGGTCTACGACCCGCGAAATCTCGAGGAACCACAACAGAAACCATTGTCT |
| | | GTACGAGCGACAGCGTATGTACTCCGTGGCTGGTCTACCTCGGTAATG |
| 344 | 39.40% | GGGTAGTTTTTTCTCCAAGGATCCCCTTAACTAGGGTGAAGATTGGGATTAAACCTAAGAT |
| | | AAAGATATAACGGTCACTGGCGACAAGCTTACAAATTTGCGCTTTACAACAGACCAAGGCG |
| | | AAAGTAATCTTGGCCCTACTAAACCAAGGGAAATCAGTAGTAGTGTTCTCCAAATAGGCAA |
| | | GGCTAATATCTATACTGTCCCTGCATGATGTGTTAAGCCATAGGCGTGTAATGTTATTCCT |
| | | TTTCCTAACCAGCTTTTAATGTATCCTTGTGTAGGAAGAACTGCGAAGTTATGTTACTCCG |
| | | AAGCCAACCAACATGTGTCCTCTTGGCACCATGATTCGAAGGTGATATTATAAGTTATTCG |
| | | ACCGTGAAGATTACATATTACTGGATGGTGTATAAATAGACCATACGTTCATTGAAGCGTG |
| | | ACTGAAGCCGACAACGGCTTACGTAATGATTCAAAATCGGTAATAAGGATAACGGTTATAT |
| | | ATAGTAGAATTCGAGATGGAAAAACCAACTTGCTAATGACAATATTAAGGGTATATCACAC |
| | | TGTGGTTTGTAAAGTAGTCACCTATTCGTGATGCCGTGTACTTCAACTTATAGTAAAAAGT |
| | | ATTGTTTTCTAACCAGCGGTAACCTGTTGCAAAAAACCACGTTTAACCGATTGATAGCTTG |
| | | TGGTAAAGTGGCATAGAGTATACTTCCTCCATCTGTAGTACTTAATAGGTGTTCCAGTTGC |
| | | AGTATAAACCTTTCTTCGAGTATCATCACTAAGACCATTAGACATAGGATATATACAATAA |
| | | GAGCTGGAACTTGAATCTTCTAATGACAGACTTTACTAATTATAGTTCAAGCGCAGTTTAA |
| | | CTATAAATACAATTGTCAATTCATCATATGGTAGGCTAGATTCCTTTAGCCTGGCGTACAG |
| | | TGGCCCGGAGGCCTTGACCAAAACATGGTTCTGTTATATCACGAGATGGATTGACTATGCT |
| | | CGTGAATCTGGAGAGGCACTAACTTGGTAACGCCCGTACTCTACCGCAGCGGGACAGGTGA |
| | | TAGACTGTCTATGTAAATCGTCATCAATCTATATTTCAATACAACTATAAATCCAGACAAG |
| | | TATCCTTGAGATAATAGTTAATCTATCCTAACTAATAAGAAGAAAAGAGACGATACGGTAG |
| | | TAGATTAAGCTTTCGCGGAAACAAGAGGAATCTACAGAAAACACCCTAAATAAGCTATTCC |
| | | ATGCCGCCTTTGCTATGAACGAAGTACGGAAGCATGATGCTTATCAACGTCAGGAACCTAG |
| | | CTCAAATCAAGGTCTTACCAGTGACGATAACATGGGTGCGGATGGTTATTTGTGGAGAGGC |
| | | GTAATACAATGTACTTGTTTTCAGGATATCAATTTAATTTCACTTAGAATACGAGACGGCC |
| | | GACAACTTTAACGAATACATTTGCATCCCACATTAATACCTGAGTGCCGCTCATATCGTCC |
| | | TAGCACAATTTTTAACAGAAGTTTTGGTGGTGAGTAGAACAACAACATGTAGTCATCTTAA |
| | | GCGTATGAAATCTGGCTCTCAAATTCATGTTTAATAGTGTTTAATCTTTTATGTATAAATC |
| | | GTTTTTATGGTTTAGACGAAGCACTCAAAAATATAGACTGATGCCTATGACCTGTGCTATC |
| | | TTTATTTTCCAGGGCAAAGATGATCTTTCCGAGTCCATATCTTGAATGACTTCCCGCCTGA |
| | | ACCAATACCTGGTCGGAAGGAGGACTCATTAATAAACATGCATAAATGGCAGATCTGAACT |
| | | GGACGGCTGACTTATCTCACAATGTGTTCTAAAGTCCACACCGTTTCTGTACCAATGAAAG |
| | | GACGAATTATACATGCATTGGTTTGGTTAAAACCAATACTTGGTAACGATCTGGACCGGGC |
| | | GGTTAGAATGATGAATTAATGCGCCGTATGTGGAATGAAGTCCTGTTAAAATGCAAAAGGT |
| | | GGCTCTTCGAGAGTTGTTGGGTTGAATGAGAGAAACGCCACCTTCACA |
| 345 | 40.00% | TAGTATCTAGTTTCAGGTGTGCACAGAATAGTTATCCTCCTTTGTCTGTGGCTATTTGGAG |
| | | AACGTATTAGAGGAAGCATATGGCAAAATGGCCTGTACACGATAGATGGTATCATGTTTGG |
| | | AGGACGCTAGGCATTTCGCCCTAAACACCGCAACGATACCTAAAGAGCTCGTCAATGGGCT |
| | | TGCCGATTAAATACGCAAGTTTTAGTCAGTCCAGACCACATTTACCGGTAATTATGCACAG |
| | | ACAAGATATTATGCTGGTTTATAGCCCATATTTGTCTCCCCCTAAAGTGAGCTCTGATATT |
| | | TGGTTAGGTCGAGTAGTACAGTTTGCTATCTATGGATACGATGTAATTGTGCTTGAGATAC |
| | | GTGCATCACGAACATTGCTAAGCGGATTCGCAATGTTCGTGATGCATGGAGTAGTCTAAGC |
| | | AATCCAACAAGCGCCTGAATATAATTTTGTCACAAGTAAACCTTCATATTGTCTAACATAC |
| | | AGAGCTGTTTTACCCCCTCATGATCTAAATCTTTCGCTTCTTCCCAAACTGCACGCCCTAT |
| | | TCGCCTGTTAGCGCATTCAACCCTAATACAGCTGTTGTGGGGATACTCTGATTGAAACAAA |
| | | GTTCTCTATGGAAGCTTCATCATTAGGCCATACGAAATAGAATCCCCTGTTGTCCAGGTGC |
| | | TTCTCGACTGCGTTGCGGTTCTTATTTTGGCTTTGCTAATAGGAACTTCTCTCTTCGAGCT |
| | | CGGTCGAACGCCAGTTCGTCAACTATACCGCCTTCTTTTTGCGCAAGGTCATCGAAACTGA |
| | | GGTCCATCCTGGGACAAGAGATCAGTTAAGCCTACACTTGTGTGAGACTCCGCAGAAAATC |
| | | GGGACCAAAGCGTTAGGGCTTCCCAATTATGAGGATCTATGGTGTCATTGAAATTGATAAT |
| | | CCTTATAGGGCCATTTTTATCCCTGACCTGAATTCTATTTGGTGAATAAAGTATTGGTCGC |
| | | CTTTCGAGGGATACTACTATGTTATGGACCTAATGGATGACCATCTGGAACATTAGCAACA |
| | | GCAACTCTAATCTTATTTTATCATCTTCAGTGTAATATATCGTACATTTTAGGCTTTCCTT |
| | | TATGTTAAATTGTTATTATGAAAGAGGTGTATTATAAGCTAGTTAAGCGCGTTAAAACACA |
| | | AGTGGTCTGCTGTCATTCATATACCAAAGAAGGTCTTGATGGACAATGTCTTCACAAGACC |
| | | ATGCATAGATTCTAAATCGATATGACACCTAACAAATGCGGGCTAATATTCGATTTCTGAC |
| | | TCCCACACTGTGAGCACGTTTATTGCGGAGACTTTTAAGCGAGATACTCTTACTCCCCATT |
| | | GCCATATATGTAAAATGGACTTCCAATTCTGCATATTTCAGTACATCCGGACTGCGTTATA |
| | | AGCATTGTCGTGGATGCATCACCATCCCATAGTTCCACTTCTTTTTTTTAGTTCAGATCCA |
| | | AACTACACTATAGGGTGACTTATTGTCGATCAAAATTATTATATGTAAGTAATAGATCATA |
| | | CATCAAGACCGAGGTCTTTGTCCAATAGAAATAGTATGTCCTGGAGTTTTATCAAATACCT |
| | | GCCATGTGCAAGTTCACAGAATAGGACGCTTCTACAGAATTCATAAAATCCCACATCCTTA |
| | | GCGTAAGTTGTCAGATGAATTAATTATATTTTTGATACGGCCCCAGTTATTCTCGAAGTCC |
| | | ACTCTTAAAAAAAGTTATTGTACGAACTTGCATAAATCGATAACCTGTTACCAACATGCCC |
| | | CGGCATAAATCAACAACGTGGTTCGGATACGACAATATCAATCAATCCGAAATTCAAAATA |
| | | GAATATTCAACTTGACTTAATCGCAGTTCATTCGTGAATAGACACATATTAGCTCTCGCGC |
| | | GCTTTCTTATCTTCACAGCTTCTTCTCGATACCTGAATAAGTACGGGACCATTTATGTTCA |
| | | TAAGCATTCAGTGAAACTGCAGTCTAAATACTATTGGCATATACTTAT |
| 346 | 40.20% | GATATGCCATCTATCGAGGCCTGTTAGCTTAGGACATTACATGACAGTGAGACCTAGATAT |
| | | ATAGTTGCATGAGTAGATGTAACCGAAGGTACTCAGGGACAGAACTGACGGATTGACGTTT |
| | | TTCAGTATCGTAAAAGTTTGAGATCCAACAATGAAAGCTTGATGCGCCAGATGATGGAAAT |
| | | GCGCAAACTGTCGTGTGATAACACGGGAATTGGTGCTAAGCTGGAATGGTCTAATTCAAGT |
| | | TCCAATCCATATCCATCTATGTGCGAGGAATTTGTAACGGTAATTATATTGCCTTACAATT |
| | | ATTATCAACCAACACACTTGAACGATGTAATTGGGGGTATATACCAATAATAGTACTGCCA |
| | | ACTACTGTTTTTTGCAAGAATTAATCGTAGTCCGAATTAAAAGAAAAGACGGTGTACGCAA |
| | | CCCAAGTAATTAAACGAATAATCATACGGTCGATATGCTCATTCGATAAAACGCGAGATCT |
| | | TTAAGTTCTCTCACCGGGGTAATGCATAATTGCCTTAATTGGAAATTGCTTTAGGTGAGAG |
| | | TCAGTAAACCATTGGTGAGATGTGGTTATACTGCACCTCACGCAAATTAATATTCTAACTT |
| | | TAACCTGAATTATGGGTTCCCCTCATCGGGAAGTATATCTAGTGCCAACCTATCACAGTTG |
| | | CGCACATATGTTTAGAAATGGTTAGTCGGTCAGGGGAACTCACGTAAGCGGTAGTAGTAGA |
| | | ATTTAATTTATGGTCTCCTAAAGCATCGACATAGTACACTGCGACCATTCTAACACATACT |
| | | AAACTTTGAACTTACTGATATCTTTTATGTTTGACTTCCTTGCTACGCAAGTCCAGGCCCA |
| | | GACAGCTGAGTTGTCCTTACACGAGCTATTTGCTGATCATATGGTTTAATCGGCACGCGAA |
| | | TTGCAAGTTTGATTTAAGGTGAGCGCATACTTGAATACAGCCAGGGAGCTCCCTACTCAGC |
| | | GATCGTCTTCAGAGATTTCACGAAAATATAAGCATTCCCATCAGAAATTCTAATTAAACCT |
| | | TACCGGAGGTGGGGATTACTCGCAGAGTTAAATAATGAGCCCACATTATGCGTTTGCTTCT |
| | | GGAGATTATGGGTGGTTTTTCCCGTACCGCCTAATATAGTATGCTTCGACTCAGCAACTTC |
| | | ACTCTAAACCCTAGAGAGCCTCTGTATGTACGCGCGTGGATGAAATCAAGAATGGTTGGAG |
| | | TCAATGACTGGGGCACAAGTGTAATCTGGTTCGATTAATACATGGCACTAGGTGCTACGAG |
| | | GACGAGTGAATGCAATATATGAGTCCTTGCTAATAAGCATCGAAGATACTCTCCGGTACTC |
| | | CTTCATATTCGACTAATCGGTGCACTCAACTTTAGGGGGGCTCCTTATTATAAAATAGATA |
| | | TAGGGTTTGTTTAAATGATTTGTTCTATTAATACGGGGAAAATTAATGCAATGTTCACCTA |
| | | GGCACGTTGGTACTCGCCGCCAAACATTGGCATTAATGGGGATACTTAGAAACAACATAAC |
| | | ATGAAAAATATCTAGGAACGCCAACATATACGCCGTGACCGTCTGTCTTAATAGACTCTTT |
| | | TTGTTTAAAGGGTACTGAGTGATTAACTAATGCTTTCCAATCCTTTCCGTTAGAAGGCTAT |
| | | TACTACAAGTGTTTCCCACGTGCCGTTAAAAATAGAATTATCTTTGTGGGTTTACGAGCGC |
| | | GTACTGAAAACAGGTTTCTTGGATGGGATAATATTATAGATAGCAATAAAGTAAACTGGAA |
| | | AACAGTATTGGATAGCATGTGATGGACCTTGACCCCCTTGTGGCATAAGATAATCTCAGCG |
| | | TTTCGTTACACTTACATTCACTGTTAATGTCTATAGGCAAGTTACTATTTGGAGTATTTCA |
| | | AAGTGAACGGAAGAAATAGAAGTGCTAACAAACTCCGTCATAGTAGGATCATATCTCCAGA |
| | | GCGACCTCATACATGCTAAAAACCTAGTAGACTTCGTACTATGGATTT |
| 347 | 40.60% | AAGACACTTTACCACATAAGTAAACCGTTGACATTATCGTGGCGGAGAGATACTGCTTGTA |
| | | CTGGGACACTCAGTATTTTGTGGAATATTGTACCTAGCGCCTCGTTCCGTGAAAGTGTGGC |
| | | ATGGATTTTCATAATTTTATGCTGTCCTGATTGCCTACAATTAATCCAGTAAGCACTAGAG |
| | | AAATATCTGCTCCTATGCTGAGATTAGCCTTATGAGGTCTTTATATCTTTCTGTAAAGGCC |
| | | ATTGTTCTTTTGATCCTGGAGTCTCTGAATTTTGATTTGTCCCTCAAAGCCTTATGTGTAC |
| | | CCGGTCCCGGAGCATGAAGACGTATATCTTGAAGTAATCCGAAAGTATTTAGGTGTCGTTG |
| | | TCCAGTAGTAATCCCGGTTATGGGTTATAATTAAGTGTTAACATCCGAGCTTGGTCTGTAT |
| | | AATAGTGTGTTTGAATAGTAAATATCAGGACTCTACAGGGACCTATTCTACTTCGGGTTGT |
| | | GTATCTTCCTTGGAATAACTTTTGCTACGCAAAAAAGCTATAACAAGGTCTGGAGACGGAT |
| | | GTGATTTAGTAGGGCAAATAGATTTAGGTCTTCGATAGTACAGAATACTATGCTACAACCA |
| | | ATCTCTTCATGGCTTTATCAATACAATGTTCTTCCTTAACTCAGACGGGAGCAATTATAGT |
| | | TAGCTGAAGGTTGCCTCACAATATGTGTCAGAGCTAGCGAAAAGCTCCTACCAATATACAT |
| | | CAGATAAGGAGTTCATACATCTGTGGCCGATCAAGCAAGCAAGGCCGTCCGGTTCACGACC |
| | | TGGGTAGTCTGAGTTTGGAGGAGAAGCCATCGCCTCTCGCATTCTACTAGAGAAAGATTTC |
| | | ACACTTACTGACAGAGCTACACTGGTACGACGAATCTACAAAACTAAGCAAAGTCCTAGGG |
| | | TGAGCAATGCATGGTAACTAGTACGATTGATCAGTGCGTGGTATACTATCCGGATAGTCCA |
| | | GACGTCAAGACCTAATCATCGTACGTAATTAAATAATAATGCATTCAACTCTTCGGATACG |
| | | ATATATACTTATATGCATTAACTATACTTTCTCATGCATTGTATCTAACATAATCTGTACG |
| | | GCAGAATTAATTACTAAAGTCTTAATGATTCGAATATTAATATCAATTTTATTACGAAACA |
| | | ACCAAACTGACAACGTAGAGAGGCAACTACCCAGAGTCGCCAAGAATACTGTTTACGAATT |
| | | GTAGAAAAGATGTAAGAATGTTCGGATGTCGGATTACTTAATTGCGAACGTTTGTCAAGTC |
| | | GTTGCAGGATACCCTCATCTCCTCTTCCTAGTGAATTATCTGAAAGTACTATTATACAATC |
| | | TAAATCGGATACATTCGTTTGTAACACCACATGGTTGGCTCAGCTGACCATTTACGCGCGA |
| | | TATTCTGTGCTATCCGAAGGCGTAAAAGGAATTCAAGTCAGTCTCCTCTTCGTTATGTAGA |
| | | AAGGGAGGACTCCTCCGCCGTATATTCAGCTGGCTTTAACTAGGAACATAGTTGCAGTTCA |
| | | AACAGTAGTAAATCCTGGAAGACATTTCTTGATAGTCTATCTCAGAAAAAGGGGGGTGACG |
| | | TTCATGTTTACTAAGACTTGAAATGTGGCTCCGTATCTGCAGAACCAGGTTTGGGCGGATG |
| | | CCGGCCGCCATGTAACACTGAACCTCGCAAGAAATGCACAATTGAACAAATGAATACTCAC |
| | | ATCTTATCGCTTAATGTTAAATTCAAGGCGAGACTGGCTCGAATTATTGGAGCCTATGAAG |
| | | ATGTATATTAATGCCAAGGCACCGCACATAGTAAAGACTATACTAACCAAGTGTGATATTC |
| | | AATCGATCGTTGTGGGGAATCAGGTACAGTTAGTGGCGAACAGCTTTGACATCCGTTTAAC |
| | | TTTGGCAGCACCACAAACCCTTTGCGTACGTTTTTGTGTTATAACCAAGTTATGTTGCAAC |
| | | CTACTTTGACCTCTTATTTCTTTGCCGCAAGACTGAATGTCGTATTAT |
| 348 | 41.50% | GAGCAACCTACGGATATACTATCGATTCTGGACATGGTAAGTGTGTTGCGTGGTTAATAAA |
| | | AAGATTTCGTGGTCGGGGGTAGATATACCTGTAAGGTTTCGAACAGACCGCTTTGTAGAAA |
| | | GAGACTTAGTCCCTTTGCAAAATGAGGGGACCGACTAAGAAAGCGTTGAATTCAGGTAATA |
| | | CTTTTTGACGTTACCATAGTTGTTGCAGTCCCGGAGTTAAACAGAGACACATCGTGGCGGA |
| | | GTCCGTAGTATCGCATGCGTGGATTTATTGTTGTAATCAGATGTTCAATATGGCGTCAATA |
| | | TACAAATAAACAGGTCAGATGGAGTTAGCCTTACTTAAAAAACGAAAACAATGTATGCCCT |
| | | AAGCAAAAAAACTAGATAAGGACGATCACCACAGTTTTAAGAGATCTATATGCCCCTTTGA |
| | | CATCCTTATTCTGACAATGGGCAGATCCAACTACAAGATGTCGTACCGCTAACACTTGACT |
| | | AACTAACGTCAAGTAAAAAGTTCGTTAGTCATATTATCAAGTATGGACTTATTCATCGACA |
| | | GGTTGTAATTAGCCCTCCCCTAGATTAGCTGGGCTGAACCCCTATTCCTACGCTCCCTTGT |
| | | CACATGTATTCTCTACCTCAATAGGCCGGAAACTCGCAAGCCCAAGTATAGCGTACGGATT |
| | | AAATTCGCGCAATCGCTCTTGACCATGTTAAATGCTTGCGCGTAACATCGAAAAGGAGGCA |
| | | AGACATTTCAGAAGTAACATATCAGTTGACGGCTTACGGTGCTGAGGTTTAAAATCCGACT |
| | | GATTGCTATCCTATCGCTGAGGAATGACTAACCTTGCAAATCCAAGTCTAGAACTGTCCTA |
| | | GTTCTGTACCATGCCCAGCGTTCGGATGTCAGTACGTGTATGCAGCATTTAGGAGGTGATG |
| | | TCTCCCAGTCGGTCAATAAGCTTTGCTTACCTCACGGATAACTAAGTTCATCTCCAGTGTA |
| | | CGAAGATTCTCTAGCACTAACTATTCATTGTAACTAATTGGTATCCGACTTTAAGCCATAG |
| | | TGTGGCATGACGTAAGTTATGTCAGTTCTTTGGAACTTTTTGCGCAGCTGTGTTGACGAAA |
| | | CACAGGTTGCAGGTTGGTCTAGGTAAGGGATGCACTCACTGCGATGTGATCCTTTAATGGC |
| | | CATTTAAATCTATCTCGAGTATAGCGTGTATACTTACTATGAAGCAAATTAGTATACATAT |
| | | AACAATGAATATACACATAGTGGGAGGTTGCCATTCATCCATGTAGGCATGTAATATGGCA |
| | | CCTCCTCTTTGGATACAGAGGCCCATGCCTCCGAATCACATATTTACTTAAACAGTTAACG |
| | | GAATTCAGGTATCCCGTTTCATTATTCGAAACGTCTCTGGGGTTACCTTACTTACGTTATC |
| | | TGCATGAGAATAGAGTCCATCGGCGTTTCTAACAATCAATCATGCTTGCAATTCAGCGAGT |
| | | GTAGAGGAATTGTAAGAACGCCGGATGCTCCCTTTACCTTATCCGCACAGGCCCCTACGAT |
| | | TGAACTATTGAAAGTTTTATTACAAATCTCATATATGGGGGAGCAGTTAAAGTTCTGCATA |
| | | AGAAGGACCTAGGATAATGCCATAAAAGGTTGATATGGAAATACTATTGGAATAAGAAAGT |
| | | ATATGGTGTCTATAATGGATATATGAGTAAACGAAGGCATTTCTTACACTTTGATTTCATT |
| | | AACTGTAATCTCTATTTGTGTTGGCGAATCCGGTAAACAGAGGTTTATAACTGGTTTACCT |
| | | TAGTCGAGTGTCTTAGATATACATGTCGATTCAGATCAATCCTACTCATCCCAAACGCACA |
| | | TGTCACGATACGTACTTTATACAGTAAGAGGCACAATGTGGGTGCCCTCTCTCGTCCGACT |
| | | TATTGCGGACGGAGAAATAGTTAGTACGGACTGTCACAAGTCTGTAACCACTAAAGATCGG |
| | | GCAGCTCAGACATTATTGAAGGTAGGCCAAAGTATCATTAATGCTTTG |
| 349 | 39.90% | ATTAATAAATGTCTAACGGTCTAGAAATGCACCTAATTTGCTACTGCTGAACTCCTGATTA |
| | | CTCCTCCTCGTTTATACTTGTTCATTAAGAATTTTTTCCGTCTAGATTAAGTACACGGTAA |
| | | TACACACGATTAAATACACCGCCACAGATCTTCGCTATCAATATTACATTTTGTTCACTCA |
| | | TTACGATAAGCGTGGCTTGGCTGAGTTCTAGACTTATCGTGTTAACGTCAATGAAAACTTA |
| | | TGGATTTGAAGCTACGATGCTAATCTAACTTTACCTTAAGCAAGAAAGACCTTCGTTAATA |
| | | GGACCCTTAAAGCCTGTGATGTCGGTTAAACGGTTCTAGTTTGATAGTGACGTTAGGGACT |
| | | CGGTATACATCTTAGCCGAACTGTCTAAATTACTTTAGAGAAACTTTTCCCTGGGGGAGGC |
| | | ACGTTCCGTTTATGGACCTCATTTGAGACTCAATATGTACAACTAATAGTGTGATTAGATC |
| | | CTGATTCCCATACGTATCGGCTCGCCCTTAATCAATACAGATCCGTGCTATGTCCATACTG |
| | | CGATTCCAAAGGTTGTCTAACAAGACAAACTTGAGAGAGGCTTCACAAAGCAACCCAGCAC |
| | | CCTTGTCCTCTTTTTTAGGGGTACGCTGACATCTGGATGCATTAAGAAATACGTATCTAGA |
| | | AGGATCGCGATAAGTCGCACAAGTTTACCACCTTATATTCTGCAGGCTGCTATTGGAGGTA |
| | | ATACGTGCTCGCACACGCCCAAGTGAGGCATTCTTACAAGACTTACCTTACAGCCTATTAA |
| | | TAACGTCGAATTTTGCGCAGCAACCAATTCCAGGGCAAACTATAAGCCTTATTGAGGTTAA |
| | | TAGGGCGCAATATATTTACGATAGAAGGTAAATCTATAATACTGTCACTTGTCAATGATGA |
| | | TGGTCTAACTAATTGATTCCCATGCAAGTGGCGAACCAGGCTTACTTTAGTTTAATAGCGA |
| | | TCAAGTATACTAAGCACACACTGAATGTATCACATAAGATACGTAAAATAAATCAACTCAT |
| | | TAAATCAAAGACAGATTCACAAATGTTTCGTGTTTTAACAGATCTGAATATAAACTCTGCT |
| | | GATGTGATCGTAGGACGTAAGAAGGTATAGTTGAAGAATAGCGTGAATATCTGATCTCTGT |
| | | TAGCAAATACATCACGATTATCACCAGGTTTACCACAACAATAAGATTGTGACTGACACTA |
| | | CTTTCTATATGAATGTATTCTCATGAGGATGCGTAAGACGTATAGGATCATACTGAATTAT |
| | | AACTCCATATTAGGGTCTATATCACATACATCTCCAAGTTAAAAAGTCTATTGGCGATTCC |
| | | ACACAACTCGCGCTAGTAGTACATTTTACCGGTACCGGTACAGTCTAAGTTATTGATCTAG |
| | | GTTCAACTTCTAAAATACTGAAGTCTCAGGTATATAGAATTTATACTACTCGCGGGACGTA |
| | | AAGCCCCTCTGTGGTTAGCGTCGCAGCGTCGAGTAAATTCCTTATAGAGCCTAAACCTTGA |
| | | TAATTTCGACGTACCGTTATAACGCAATTAATAGACTTCTCATTTTCCTGCCGAGTCGGGT |
| | | CTGGTATAGTCTAGGACGGGGGTAGATATGATCGTCGTCTTCTCTAATCTAATTTAATCTA |
| | | TAACCACAGCGTACAAGTAAGGTATGTAAGATACAGAGATAAATTAGAGATTTGTGTTACT |
| | | CCGCATGTTGAACTAAACCCAAAGGTTCACGCCGTATGCCTTTGAAGTTCCTCCGCTGAAA |
| | | AGGCTCCGGGTGTCCCCTACCCGATATGGCGGAAATCGTTAATTCTCATAACGACCAACCT |
| | | TACCTTGGACACACCTAAGCACTAAGTCGGTAAATGGAGTACACAATGTGGGAGTTGTGTT |
| | | TAACATAATGAGGCTCGTTCAGACTATGTTCGAGGCGTATAACGATTTGTGACAGATTCCT |
| | | CATCAACTCGGGTCAGATTTATAGGAATGGTAAATTCCCTATATCCTA |
| 350 | 39.60% | TATGGTGTGGCACATATGAATAAAACAAGGAGAAGCAGCCGACAATACTTAGAACGTGTCA |
| | | GAACAATCAAGATGTCTGAAACGTTCAACAATCGAGTTATTCCGGGCTAATTTATTCCCAT |
| | | CCTTATATACAGAGCCGCACAATACCAAGTAACGTGCTTTGGGCCACGAACTCACTCTAGT |
| | | CTTCCGGACCCTCCGGTACTACTCGGTATGGTGGATATTCATGAGAATGGTTTTAGTCTTA |
| | | AAAAAATGTGAACAAGAAAACATTTACGTCCAAGAAAGCGGTATTTTGTTTGGGTCTAGGA |
| | | AACAATCAGTCGTGGACCTGGGCGAGATCGGCTGTTTTCGACCGATTTTATGCTAAGCAGA |
| | | AGGAAGTGACCGAGGTTGTGTTTAGATCCAGTAAAAGTCGTCATACCCGAGGAGATTTCTG |
| | | TGGTGCCTAGTGACTAGCGATCCCGTGCAGCAGTTCAAATGCGCTGGATAGTTCGCTCCTG |
| | | CACCACTAGTTCACACCAGAAGTATGTCTTTTAAGAGACTGTCTAAGAAATATAGTCTCTA |
| | | AACGTGACTATCGTTCACTCCCTGTACAAATCTAGGACTAACGGGTATAGATTAAACGTAT |
| | | TAGAATTTCGGAGCATTAGAATTTTGTTGTTCTAAGTTAGGATGATTTCAAGTGTCCATGT |
| | | AAATTGAGGTCAATATAGGACGATCTACATCCGAGATAGGCCAAGTACGATTCTGTGTTAC |
| | | ATTTTGCGTTCGCACAAGCTAGGACGAGGGTATGAGCATTTTGTGCTAACCGAATGAGATG |
| | | CAGCTTATTGTATCCTTACCCGCAACATAGGGCATGAAGGCGTGGTTCGAGAATCGCGCGA |
| | | GATAAATACATGTTTCGATTTATGTCAACCACTGCAATGGTTTATAAATGTTATTCAAGCA |
| | | TCGATTCAATAACCTCTGGATGTAGTAATATCTGCGGGTGTGTAAGTGCGATATCCTAAGT |
| | | CGGGAGATTTAACAATACCTTGGGATGCTCCGGACAATTTTCGACGTACGCAATTATGAAC |
| | | ATGCATTGATTGACTAAACTTAAGAAACATAATCAGTGTATAGTATTGTAACAATGGATTC |
| | | TGAGTGTCTAATGTTTTCTCGCTCCATGTTATAACACATAATTATACTTATAATACCATCC |
| | | CATCTTTAAGTACAAAACCTTGTTGCGCTGCTTTATGGAGACTATTGAGCCCAACGGGTTG |
| | | AGTGGTTATTACTATTTGAAGTAAAAGCAGTATCTACTCAGATTCCTAGAGGTAAATATGA |
| | | ACTTGTTTTCTATCTGGTTATCTATTTTTAGTTTTATGGATATGGACGAAGTTAAAAGTTA |
| | | TAGACCTGACATTCTTCTCCCATAGGTATAGTAGTGGAGTTAAACAAGTTCTTAGTGGGGG |
| | | AAATGACGTACAGACTACTATCTTGATGATAGCTTTTCGATCAAAGAAGAGTTTCAACCGC |
| | | TGTAAAGGTTTATATGCGATGTAGTGTGGTACGATAACGTACTTTGCCGATCATTCACTGA |
| | | TTCCATTAGGTACGACACTCTCAGTTACAAAGCGGTACTAACCTAGCAAAAAGTGAATATC |
| | | GCCCTACAAACTATTACTGGAGTGCGGTGGCAGCTTTGGCGAAAATTGGCCGAACTCTTTG |
| | | CTGTTTATATGGTAACTATTCTCACTATGCTACTGATTGGAAAAAGATATTTGCCAACTAA |
| | | TAGTCGTAATGTTAGTATTGATAGGGATTATAGGCATTTAAAGTTCCCTGAAACATACGGT |
| | | AAATAAGATCTCTTTTAACAAGACCAGGGGTGGCTCACTGGGGTAGCAAATACTTAACGAT |
| | | CCCTTTTTCATCAAGTGAGTTATCTGCTTTGGATTCTTACAACTAGATGTTATAAAGAAAG |
| | | AAGCTGCGCAGTTTGCATGACTAAAATTTATATGAAGTAGTAGTTATTAGTACTATCTCTT |
| | | AGTAGGCTAGAATGTAAACCTGCAGACATCATGGAATGCACATACCCG |
| 351 | 38.40 | TCAATAGCCCAGTCGGTTTTGTTAGATACATTTTATCGAATCTGTAAAGATATTTTATAAT |
| | | AAGATAATATCAGCGCCTAGCTGCGGAATTCCACTCAGAGAATACCTCTCCTGAATATCAG |
| | | CCTTAGTGGCGTTATACGATATTTCACACTCTCAAAATCCCGAGTCAGACTATACCCGCGC |
| | | ATGTTTAGTAAAGGTTGATTCTGAGATCTCGAGTCCAAAAAAGATACCCACTACTTTAAAG |
| | | ATTTGCATTCAGTTGTTCCATCGGCCTGGGTAGTAAAGGGGGTATGCTCGCTCCGAGTCGA |
| | | TGGAACTGTAAATGTTAGCCCTGATACGCGGAACATATCAGTAACAATCTTTACCTAATAT |
| | | GGAGTGGGATTAAGCTTCATAGAGGATATGAAACGCTCGTAGTATGGCTTCCTACATAAGT |
| | | AGAATTATTAGCAACTAAGATATTACCACTGCCCAATAAAAGAGATTCCACTTAGATTCAT |
| | | AGGTAGTCCCAACAATCATGTCTGAATACTAAATTGATCAATTGGACTATGTCTAAATTAT |
| | | TTTGAAGAAGTAATCATCAACTTAGGCGCTTTTTAGTGTTAAGAGCGCGTTATTGCCAACC |
| | | GGGCTAAACCTGTGTAACTCTTCAATATTGTATATAATTATAGGCAGAATAAGCTATGAGT |
| | | GCATTATGAGATAAACATAGATTTTTGTCCACTCGAAATATTTGAATTTCTTGATCCTGGG |
| | | CTAGTTCAGCCATAAGTTTTCACTAATAGTTAGGACTACCAATTACACTACATTCAGTTGC |
| | | TGAAATTCACATCACTGCCGCAATATTTATGAAGCTATTATTGCATTAAGACTTAGGAGAT |
| | | TAATACGAAGTTGATATATTTTTCAGAATCAGCGAAAAGACCCCCTATTGACATTACGAAT |
| | | TCGAGTTTAACGAGCACATAAATCAAACACTACGAGGTTACCAAGATTGTATCTTACATTA |
| | | ATGCTATCGAGCCAGCCGTCATGTTTAACTGGATAGTCATAATTAATATCCAATGATCGTT |
| | | TCACGTAGCTGCATATCGAGGAAGTTGTATAATTGAAAACCCACACATTAGAATGCATGGT |
| | | GCATCGCTAGGGTTTATCTTATCTTGCTCGTGCCAAGAGTGTAGAAAGCCACATATTGATA |
| | | CGGAAGCTGCCTAGGAGGTTGGTATATGTTGATTGTGCTCACCATCTCCCTTCCTAATCTC |
| | | CTAGTGTTAAGTCCAATCAGTGGGCTGGCTCTGGTTAAAAGTAATATACACGCTAGATCTC |
| | | TCTACTATAATACAGGCTAAGCCTACGCGCTTTCAATGCACTGATTACCAACTTAGCTACG |
| | | GCCAGCCCCATTTAATGAATTATCTCAGATGAATTCAGACATTATTCTCTACAAGGACACT |
| | | TTAGAGTGTCCTGCGGAGGCATAATTATTATCTAAGATGGGGTAAGTCCGATGGAAGACAC |
| | | AGATACATCGGACTATTCCTATTAGCCGAGAGTCAACCGTTAGAACTCGGAAAAAGACATC |
| | | GAAGCCGGTAACCTACGCACTATAAATTTCCGCAGAGACATATGTAAAGTTTTATTAGAAC |
| | | TGGTATCTTGATTACGATTCTTAACTCTCATACGCCGGTCCGGAATTTGTGACTCGAGAAA |
| | | ATGTAATGACATGCTCCAATTGATTTCAAAATTAGATTTAAGGTCAGCGAACTATGTTTAT |
| | | TCAACCGTTTACAACGCTATTATGCGCGATGGATGGGGCCTTGTATCTAGAAACCGAATAA |
| | | TAAGATACCTGTTAAATGGGAAACTTAGATTATTGCGATTAATTCTCACTTCAGAGGGTTA |
| | | TCGTGCCGAATTCCTGACTTTGGAATAATAAAGTTGATATTGAGGTGCAATATCAACTACA |
| | | CTGGTTTAACCTTTAAACACATGGAGTCAAGTTTTCGCTATGCCAGCCGGTTATGCAGCTA |
| | | GGATTAATATTAGAGCTCTTTTCTAATTCGTCCTAATAATCTCTTCAC |

In one embodiment, the first stuffer has a sequence comprising at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in Table 2. In another embodiment, the second stuffer has a sequence comprising at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in Table 2.

It is preferable that the stuffer sequence not interfere with the resolution of the cleavage site at the target nucleic acid. Thus, the stuffer sequence should have minimal sequence identity to the nucleic acid sequence at the cleavage site of the target nucleic acid. In some embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 nucleotides from the cleavage site of the target nucleic acid. In some embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 base pairs from the cleavage site of the target nucleic acid.

In order to avoid off-target molecular recombination events, it is preferable that the stuffer sequence have minimal homology to a nucleic acid sequence in the genome of the target cell. In some embodiments, the stuffer sequence has minimal sequence identity to a nucleic acid in the genome of the target cell. In some embodiments, the stuffer sequence is less than 80%, 70%, 60%, 55%, 50%, 45%, 40%, 35%, 30%, 25%, 20%, or 10% identical to any nucleic acid sequence of the same length (as measured in base pairs or nucleotides) in the genome of the target cell. In some embodiments, a 20 base pair stretch of the stuffer sequence is less than 80%, 70%, 60%, 55%, 50%, 45%, 40%, 35%, 30%, 25%, 20%, or 10% identical to any at least 20 base pair stretch of nucleic acid of the target cell genome. In some embodiments, a 20 nucleotide stretch of the stuffer sequence is less than 60%, 55%, 50%, 45%, 40%, 35%, 30%, 25%, 20%, or 10% identical to any at least 20 nucleotide stretch of nucleic acid of the target cell genome.

In some embodiments, the stuffer sequence has minimal sequence identity to a nucleic acid sequence in the donor template (e.g., the nucleic acid sequence of the cargo, or the nucleic acid sequence of a priming site present in the donor template). In some embodiments, the stuffer sequence is less than 80%, 70%, 60%, 55%, 50%, 45%, 40′%, 35%, 30%, 25%, 20%, or 10% identical to any nucleic acid sequence of the same length (as measured in base pairs or nucleotides) in the donor template. In some embodiments, a 20 base pair stretch of the stuffer sequence is less than 80%, 70%, 60%, 55%, 50%, 45%, 40%, 35%, 30%, 25%, 20%, or 10% identical to any 20 base pair stretch of nucleic acid of the donor template. In some embodiments, a 20 nucleotide stretch of the stuffer sequence is less than 80%, 70%, 60%, 55%, 50%, 45%, 40%, 35%, 30%, 25%, 20%, or 10% identical to any 20 nucleotide stretch of nucleic acid of the donor template.

In some embodiments, the length of the first homology arm and its adjacent stuffer sequence (i.e., A1+S1) is approximately equal to the length of the second homology arm and its adjacent stuffer sequence (i.e., A2+S2). For example, in some embodiments the length of A1+S1 is the same as the length of A2+S2 (as determined in base pairs or nucleotides). In some embodiments, the length of A1+S1 differs from the length of A2+S2 by 25 nucleotides or less. In some embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 nucleotides or less. In some embodiments, the length of A1+S1 differs from the length of A2+S2 by 25 base pairs or less. In some embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 base pairs or less.

In some embodiments, the length of A1+H1 is 250 base pairs or less. In some embodiments, the length of A1+H1 is 200 base pairs or less. In some embodiments, the length of A1+H1 is 150 base pairs or less. In some embodiments, the length of A1+H1 is 100 base pairs or less. In some embodiments, the length of A1+H1 is 50 base pairs or less. In some embodiments, the length of A1+H1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In some embodiments, the length of A1+H1 is 40 base pairs. In some embodiments, the length of A2+H2 is 250 base pairs or less. In some embodiments, the length of A2+H2 is 200 base pairs or less. In some embodiments, the length of A2+H2 is 150 base pairs or less. In some embodiments, the length of A2+H2 is 100 base pairs or less. In some embodiments, the length of A2+H2 is 50 base pairs or less. In some embodiments, the length of A2+H2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In some embodiments, the length of A2+H2 is 40 base pairs.

In some embodiments, the length of A1+S1 is the same as the length of H1+X+H2 (as determined in nucleotides or base pairs). In some embodiments, the length of A1+S1 differs from the length of H1+X+H2 by less than 25 nucleotides. In some embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In some embodiments, the length of A1+S1 differs from the length of H1+X+H2 by less than 25 base pairs. In some embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 base pairs.

In some embodiments, the length of A2+S2 is the same as the length of H1+X+H2 (as determined in nucleotides or base pairs). In some embodiments, the length of A2+S2 differs from the length of H1+X+H2 by less than 25 nucleotides. In some embodiments, the length of A2+S2 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In some embodiments, the length of A2+S2 differs from the length of H1+X+H2 by less than 25 base pairs. In some embodiments, the length of A2+S2 differs from the length of H1+X+H2 by 24, 23, 22, 21, 20, 19 18, 17, 16, 15, 14, 13, 12, 11, 1, 9, 8, 7, 6, 5, 4, 3, or 2 base pairs.

E. Donor Templates Generally

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs or to boost overall editing rate, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g., flanking or adjoining) a target sequence to be cleaved. These homologous regions are referred to here as “homology arms,” and are illustrated schematically below:

[5′ homology arm]-[replacement sequence]-[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson 2016, which is incorporated by reference herein, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or then, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino, which is incorporated by reference.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

In certain embodiments, silent, non-pathogenic SNPs may be included in the ssODN donor template to allow for identification of a gene editing event.

In certain embodiments, a donor template may be a non-specific template that is non-homologous to regions of DNA within or near a target sequence to be cleaved.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g., administered to a subject) immediately, or they can be maintained or stored for later use. Those of skill in the art will appreciate that cells can be maintained in culture or stored (e.g., frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 3 and 4 set forth several, non-limiting examples of genome editing system implementations. Those of skill in the art will appreciate, however, that these listings are not comprehensive, and that other implementations are possible. With reference to Table 3 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 3
Genome editing components

| RNA-guided | | Donor | |
|---|---|---|---|
| Nuclease | gRNA | Template | Comments |
| Protem | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded or |
| | | | double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both a |
| | | | gRNA and a donor template. |
| DNA | | | A DNA or DNA vector encoding |
| | | | an RNA-guided nuclease, a gRNA |
| | | | and a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding |
| | | | an RNA-guided nuclease and a |
| | | | gRNA |
| DNA | | DNA | A first DNA or DNA vector |
| | | | encoding an RNA-guided nuclease |
| | | | and a gRNA, and a second DNA or |
| | | | DNA vector encoding a donor |
| | | | template. |
| DNA | DNA | | A first DNA or DNA vector |
| | | | encoding an RNA-guided nuclease |
| | | | and second DNA or DNA vector |
| | | | encoding a gRNA and a donor |
| | | | template. |
| DNA | | | A first DNA or DNA vector |
| | DNA | | encoding an RNA-guided nuclease |
| | | | and a donor template, and a second |
| | | | DNA or DNA vector encoding a |
| | | | gRNA |
| DNA | | | A DNA or DNA vector encoding |
| | RNA | | an RNA-guided nuclease and a |
| | | | donor template, and a gRNA |

| | | |
|---|---|---|
| RNA | [N/A] | An RNA or RNA vector encoding |
| | | an RNA-guided nuclease and |
| | | comprising a gRNA |
| RNA | DNA | An RNA or RNA vector encoding |
| | | an RNA-guided nuclease and |
| | | comprising a gRNA, and a DNA or |
| | | DNA vector encoding a donor |
| | | template. |

Table 4 summarizes various delivery methods for the components of genome editing systems, as described herein. Again, the listing is intended to be exemplary rather than limiting.

TABLE 4
Delivery vectors and modes

| | | Delivery | | | |
|---|---|---|---|---|---|
| | | into Non- | Duration | | Type of |
| | | Dividing | of | Genome | Molecule |
| Delivery Vector/Mode | | Ceils | Expression | Integration | Delivered |
| Physical (e.g., | | YES | Transient | NO | Nucleic Acids |
| electroporation, particle gun, | | | | | and Proteins |
| Calcium Phosphate | | | | | |
| transfection, cell compression | | | | | |
| or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 4, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 4, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 5, and Table 6 lists exemplary polymers for use in gene transfer and/or nanoparticle formulations.

TABLE 5
Lipids used for gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl- | DOSPA | Cationic |
| 1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3- dimethylaminopropane | DLinDMA | Cationic |
| 2,2-dilinoleyl-4-dimethylaminoethyl-[1,3]- dioxolane | DLin-KC2- | Cationic |
| | DMA | |
| dilinoleyl- methyl-4-dimethylaminobutyrate | DLin-MC3- | Cationic |
| | DMA | |

TABLE 6
Polymers used for gene transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system. e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see. e.g., Lee 2012). Lipid-mediated transfection, peptide-mediated delivery, GalNAc- or other conjugate-mediated delivery, and combinations thereof, can also be used for delivery in vitro and in vivo. A protective, interactive, non-condensing (PINC) system may be used for delivery.

In vitro delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrvlate, acrylic and methacrylic acids, and others such as poly(vinyl alcohol), poly(vinyl pyrolidone), and poly(vinyl acetate): poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### EXAMPLES

The principles and embodiments described above are further illustrated by the non-limiting examples that follow:

### Example 1: Targeted Integration at HBB Locus

Previously, it was thought that longer homology arms provided more efficient homologous recombination, and typical homology arm lengths were between 500 and 2000 bases (Wang et al., NAR 2015; De Ravin, et al. NBT 2016; Genovese et al. Nature 2014). However, the methods described in the instant example can surprisingly be performed using donor templates having a shorter homology arm (HA) to achieve targeted integration.

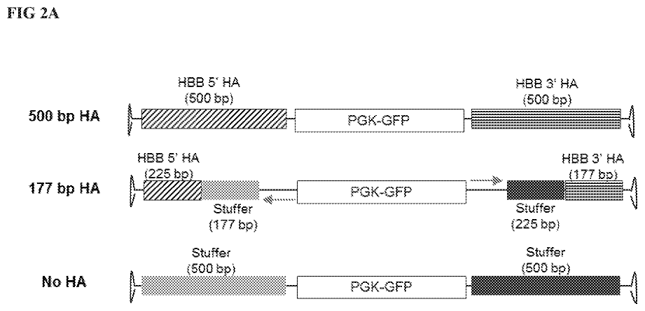

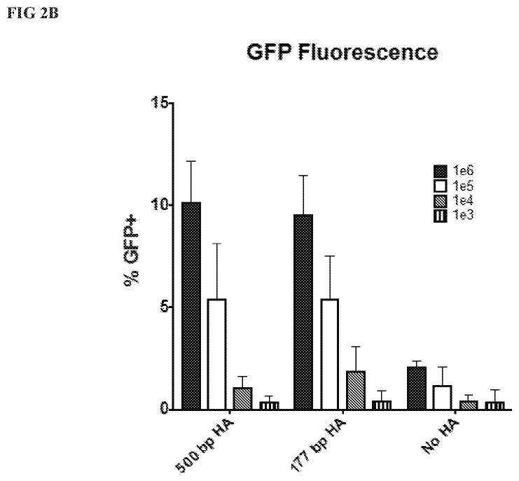

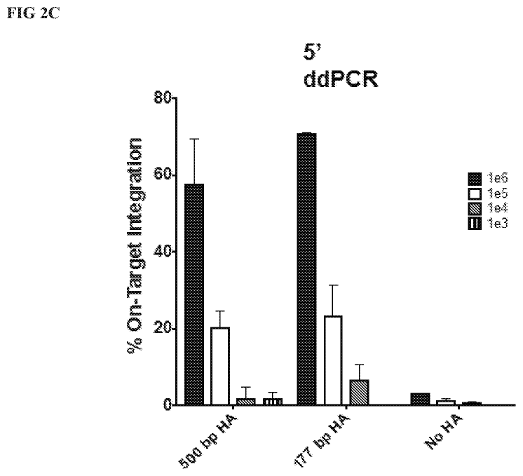

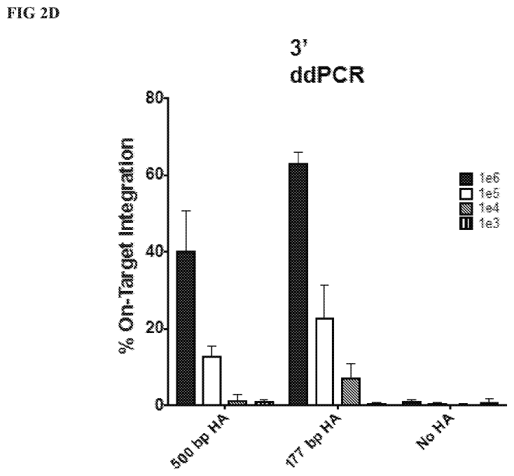

To test whether shortening the homology arms negatively impacted targeted integration efficiency, two AAV6 donor templates to the HBB locus were designed (FIG. 2A). The first donor template contained symmetrical homology arms of 500 nt each, flanking a GFP expression cassette (hPGK promoter, GFP, and polyA sequence). The second donor template contained shorter homology arms (5′: 225 bp, 3′: 177 bp) in addition to stuffer DNA and the genomic priming sites, as described above, flanking an identical GFP cassette. A third donor template having 500 nt of DNA that was non-homologous to the human genome 5′ and 3′ of the same GFP cassette was used. The 5′ and 3′ stuffer sequences were derived from the master stuffer sequence and comprised different sequences in each construct to avoid intramolecular recombination.

Table 7 provides the sequences for the master stuffer and the three donor templates depicted in FIG. 2A. A “master stuffer sequence” consists of 2000 nucleotides. It contains roughly the same GC content as the genome as a whole, (e.g., ˜40% for the whole genome). Depending on the target locus, the GC content may vary. Based on the design of the donor templates, certain portions of the “master stuffer sequence” (or the reverse compliment thereof) are selected as appropriate stuffers. The selection is based on the following three criteria:

1) the length

2) the homology, and

3) structure.

In the second exemplary donor template design depicted in FIG. 2A (HA+Stuffers), the stuffer 5′ to the cargo (i.e., PGK-GFP) is 177 nucleotides long while the stuffer 3′ to the cargo is 225 nucleotides long. Therefore, the 5′ stuffer (177 nt) may be any consecutive 177 nucleotide sequence within the “master stuffer sequence” or the reverse compliment thereof. The 3′ stuffer (225 nt) may be any consecutive 225 nucleotide sequence within the “master stuffer sequence”, or the reverse compliment thereof.

For the homology requirement, neither the 5′ stuffer nor the 3′ stuffer have homology with any other sequence in the genome (e.g., no more than 20 nucleotide homology), nor to any other sequence in the donor template (i.e., primers, cargo, the other stuffer sequence, homology arms). It is preferable that the stuffer not contain a nucleic acid sequence that forms secondary structures.

TABLE 7
Nucleic Acid Sequences for the Master Stuffer and Donor Templates.

| | | SEQ |
|---|---|---|
| DESCRIPTION | SEQUENCE | ID NO: |
| Master Stuffer | TACTCTTAATTCATTACATATTGTGCGGTCGAATTCAGGGAGCC | 352 |
| | GATAATGCGGTTACAATAATTCCTATACTTAAATATACAAAGAT | |
| | TTAAAATTTCAAAAAATGGTTACCAGCATCGTTAGTGCGTATAC | |
| | ATCAAGAGGCACGTGCCCCGGAGACAGCAAGTAAGCTCTTTAAA | |
| | CATGCTTTGACATACGATTTTTAATAAAACATGAGCATTTGAAT | |
| | AAAAACGACTTCCTCATACTGTAAACATCACGCATGCACATTAG | |
| | ACAATAATCCAGTAACGAAACGGCTTCAGTCGTAATCGCCCATA | |
| | TAGTTGGCTACAGAATGTTGGATAGAGAACTTAAGTACGCTAAG | |
| | GCGGCGTATTTTCTTAATATTTAGGGGTATTGCCGCAGTCATTA | |
| | CAGATAACCGCCTATGCGGCCATGCCAGGATTATAGATAACTTT | |
| | TTAACATTAGCCGCAGAGGTGGGACTAGCACGTAATATCAGCAC | |
| | ATAACGTGTCAGTCAGCATATTACGGAATAATCCTATCGTTATC | |
| | AGATCTCCCCTGTCATATCACAACATGTTTCGATGTTCCAAAAC | |
| | CGGGAACATTTTGGATCGGTTAAATGATTGTACATCATTTGTTG | |
| | CAGACCTTAGGAACATCCATCATCCGCCGCCCTTCATCTCTCAA | |
| | AGTTATCGCTTGTAAATGTATCACAACTAGTATGGTGTAAAATA | |
| | TAGTACCCGATAGACTCGATTTAGGCTGTGAGGTTAGTAACTCT | |
| | AACTTGTGCTTTCGACACAGATCCTCGTTTCATGCAAATTTAAT | |
| | TTTGCTGGCTAGATATATCAATCGTTCGATTATTCAGAGTTTTG | |
| | GTGAGGAGCCCCCTCAGATGGGAGCATTTTCACTACTTTAAAGA | |
| | ATAACGTATTTTTCGCCCTGTCCCTTAGTGACTTAAAAAGAATG | |
| | GGGGCTAGTGCTTAGAGCTGGTAGGGCTTTTTGGTTCTATCTGT | |
| | TAAGCGAATAAGCTGTCACCTAAGCAAATTAATGCTTTCATTGT | |
| | ACCCCGGAACTTTAAATCTATGAACAATCGCAACAAATTGTCCA | |
| | AAGGCAACAATACGACACAGTTAGAGGCCATCGGCGCAGGTACA | |
| | CTCTATCCACGCCTATCAGAATGTCACCTGGTTAATGGTCAATT | |
| | TAGGTGGCTGGAGGCACATGTGAAGCAATATGGTCTAGGGAAAG | |
| | ATATCGGTTTACTTAGATTTTATAGTTCCGGATCCAACTTAAAT | |
| | AATATAGGTATTAAAGAGCAGTATCAAGAGGGTTTCTTCCCAAG | |
| | GAATCTTGCGATTTTCATACACAGCTTTAACAAATTTCACTAGA | |
| | CGCACCTTCATTTTGTCGTCTCGTTGTATATGAGTCCGGGGTAA | |
| | GAATTTTTTACCGTATTTAACATGATCAACGGGTACTAAAGCAA | |
| | TGTCATTTCTAAACACAGTAGGTAAAGGACACGTCATCTTATTT | |
| | TAAAGAATGTCAGAAATCAGGGAGACTAGATCGATATTACGTGT | |
| | TTTTTGAGTCAAAGACGGCCGTAAAATAATCAAGCAGTCTTTCT | |
| | ACCTGTACTTGTCGCTACCTAGAATCTTTAATTTATCCATGTCA | |
| | AGGAGGATGCCCATCTGAAACAATACCTGTTGCTAGATCGTCTA | |
| | ACAACGGCATCTTGTCGTCCATGCGGGGTTGTTCTTGTACGTAT | |
| | CAGCGTCGGTTATATGTAAAAATAATGTTTTACTACTATGCCAT | |
| | CTGTCCCGTATTCTTAAGCATGACTAATATTAAAAGCCGCCTAT | |
| | ATATCGAGAACGACTACCATTGGAATTTAAAATTGCTTCCAAGC | |
| | TATGATGATGTGACCTCTCACATTGTGGTAGTATAAACTATGGT | |
| | TAGCCACGACTCGTTCGGACAAGTAGTAATATCTCTTGGTAATA | |
| | GTCGGGTTACCGCGAAATATTTGAAATTGATATTAAGAAGCAAT | |
| | GATTTGTACATAAGTATACCTGTAATGAATTCCTGCGTTAGCAG | |
| | CTTAGTATCCATTATTAGAG | |
| Donor template | TTATCCCCTTCCTATGACATGAACTTAACCATAGAAAAGAAGGG | 353 |
| design 1 | GAAAGAAAACATCAAGCGTCCCATAGACTCACCCTGAAGTTCTC | |
| (HA only) | AGGATCCACGTGCAGCTTGTCACAGTGCAGCTCACTCAGTGTGG | |
| | CAAAGGTGCCCTTGAGGTTGTCCAGGTGAGCCAGGCCATCACTA | |
| | AAGGCACCGAGCACTTTCTTGCCATGAGCCTTCACCTTAGGGTT | |
| | GCCCATAACAGCATCAGGAGTGGACAGATCCCCAAAGGACTCAA | |
| | AGAACCTCTGGGTCCAAGGGTAGACCACCAGCAGCCTAAGGGTG | |
| | GGAAAATAGACCAATAGGCAGAGAGAGTCAGTGCCTATCAGAAA | |
| | CCCAAGAGTCTTCTCTGTCTCCACATGCCCAGTTTCTATTGGTC | |
| | TCCTTAAACCTGTCTTGTAACCTTGATACCAACCTGCCCAGGGC | |
| | CTCACCACCAACTTCATCCACGTTCACCTTGCCCCACAGGGCAG | |
| | TAACGGCAGACTTCTCAAGCTTCCATAGAGCCCACCGCATCCCC | |
| | AGCATGCCTCCTATTCTCTTCCCAATCCTCCCCCTTGCTCTCCT | |
| | GCCCCACCCCACCCCCCAGAATAGAATGACACCTACTCAGACAA | |
| | TGCGATGCAATTTCCTCATTTTATTAGGAAAGGACAGTGGGAGT | |
| | GGCACCTTCCAGGGTCAAGGAAGGCACGGGGGAGGGGCAAACAA | |
| | CAGATGGCTGGCAACTAGAAGGCACAGTCGAGGCTGATCAGCGG | |
| | GTTTAAACGGGCCTCCTAGACTCGACGCCCCCGCTTTACTTGTA | |
| | CAGCTCGTCCATGCCGAGAGTGATCCCGGCGGCGGTCACGAACT | |
| | CCAGCAGGACCATGTGATCGCGCTTCTCCTTGGGGTCTTTGCTC | |
| | AGGGCGGACTGGGTGCTCAGGTAGTGGTTGTCCCGCAGCAGCAC | |
| | GGGGCCGTCGCCGATGGGGGTGTTCTGCTGGTAGTGGTCGGCGA | |
| | GCTGCACGCTGCCGTCCTCGATGTTGTGGCGGATCTTGAAGTTC | |
| | ACCTTGATGCCGTTCTTCTGCTTGTCGGCCATGATATAGACGTT | |
| | GTGGCTGTTGTAGTTGTACTCCAGCTTGTGCCCCAGGATGTTGC | |
| | CGTCCTCCTTGAAGTCGATGCCCTTCAGCTCGATGCGGTTCACC | |
| | AGGGTGTCGCCCTCGAACTTCACCTCGGCGCGGGTCTTGTAGTT | |
| | GCCGTCGTCCTTGAAGAAGATGGTGCGCTCCTGGACGTAGCCTT | |
| | CGGGCATGGCGGACTTGAAGAAGTCGTGCTGCTTCATGTGGTCG | |
| | GGGTAGCGGCTGAAGCACTGCACGCCGTAGGTCAGGGTGGTCAC | |
| | GAGGGTGGGCCAGGGCACGGGCAGCTTGCCGGTGGTGCAGATGA | |
| | ACTTCAGGGTCAGCTTGCCGTAGGTGGCATCGCCCTCGCCCTCG | |
| | CCGGACACGCTGAACTTGTGGCCGTTTACGTCGCCGTCCAGCTC | |
| | GACCAGGATGGGCACCACCCCGGTGAACAGCTCCTCGCCCTTGC | |
| | TCACCATGGTGGCGACCGGTGGGGAGAGAGGTCGGTGATTCGGT | |
| | CAACGAGGGAGCCGACTGCCGACGTGCGCTCCGGAGGCTTGCAG | |
| | AATGCGGAACACCGCGCGGGCAGGAACAGGGCCCACACTACCGC | |
| | CCCACACCCCGCCTCCCCCACCGCCCCTTCCCCCCCGCTGCTCT | |
| | CGGCGCGCCCTGCTGAGCAGCCGCTATTCCCCACAGCCCATCGC | |
| | GGTCGGCGCGCTGCCATTGCTCCCTCCCGCTGTCCGTCTGCGAG | |
| | GGTACTAGTGAGACGTGCGGCTTCCGTTTGTCACGTCCGGCACG | |
| | CCGCGAACCGCAAGGAACCTTCCCGACTTAGGGGCGGAGCAGGA | |
| | AGCGTCGCCGGGGGGCCCACAAGGGTAGCGGCGAAGATCCGGGT | |
| | GACGCTGCGAACGGACGTGAAGAATGTGCGAGACCCAGGGTCGG | |
| | CGCCGCTGCGTTTCCCGGAACCACGCCCAGAGCAGCCGCGTCCC | |
| | TGCGCAAACCCAGGGCTGCCTTGGAAAAGGCGCAACCCCAACCC | |
| | CGTGGAAGCTCTCAGGAGTCAGATGCACCATGGTGTCTGTTTGA | |
| | GGTTGCTAGTGAACACAGTTGTGTCAGAAGCAAATGTAAGCAAT | |
| | AGATGGCTCTGCCCTGACTTTTATGCCCAGCCCTGGCTCCTGCC | |
| | CTCCCTGCTCCTGGGAGTAGATTCCCCAACCCTAGGGTGTGGCT | |
| | CCACAGGGTGAGGTCTAAGTGATGACAGCCGTACCTGTCCTTGG | |
| | CTCTTCTGGCACTGGCTTAGGAGTTGGACTTCAAACCCTCAGCC | |
| | CTCCCTCTAAGATATATCTCTTGGCCCCATACCATCAGTACAAA | |
| | TTGCTACTAAAAACATCCTCCTTTGCAAGTGTATTTACGTAATA | |
| | TTTGGAATCACAGCTTGGTAAGCATATTGAAGATCGTTTTCCCA | |
| | ATTTTCTTATTACACAAATAAGAAGTTGATGCACTAAAAGTGGA | |
| | AGAGTTTTGTCTACCATAATTCAGCTTTGGGATATGTAGATGGA | |
| | TCTCTTCCTGCGTCTCCAGAATATGC | |
| Donor template | GTCCAAGGGTAGACCACCAGCAGCCTAAGGGTGGGAAAATAGAC | 354 |
| design 2 | CAATAGGCAGAGAGAGTCAGTGCCTATCAGAAACCCAAGAGTCT | |
| (HS + Stuffers) | TCTCTGTCTCCACATGCCCAGTTTCTATTGGTCTCCTTAAACCT | |
| | GTCTTGTAACCTTGATACCAACCTGCCCAGGGCCTCACCACCAA | |
| | CTTCATCCACGTTCACCTTGCCCCACAGGGCAGTAACGGCAGAC | |
| | TTCTCTACTCTTAATTCATTACATATTGTGCGGTCGAATTCAGG | |
| | GAGCCGATAATGCGGTTACAATAATTCCTATACTTAAATATACA | |
| | AAGATTTAAAATTTCAAAAAATGGTTACCAGCATCGTTAGTGCG | |
| | TATACATCAAGAGGCACGTGCCCCGGAGACAGCAAGTAAGCTCT | |
| | TTAAACGGTCTAAGTGATGACAGCCGTAAGCTTCCATAGAGCCC | |
| | ACCGCATCCCCAGCATGCCTGCTATTGTCTTCCCAATCCTCCCC | |
| | CTTCCTGTCCTGCCCCACCCCACCCCCCAGAATAGAATGACACC | |
| | TACTCAGACAATGCGATGCAATTTCCTCATTTTATTAGGAAAGG | |
| | ACAGTGGGAGTGGCACCTTCCAGGGTCAAGGAAGGCACGGGGGA | |
| | GGGGCAAACAACAGATGGCTGGCAACTAGAAGGCACAGTCGAGG | |
| | CTGATCAGCGGGTTTAAACGGGCCCTCTAGACTCGACGCGGCCG | |
| | CTTTACTTGTACAGCTCGTCCATGCCGAGAGTGATCCCGGCGGC | |
| | GGTCACGAACTCCAGCAGGACCATGTGATCGCGCTTCTCGTTGC | |
| | GGTCTTTGCTCAGGGCGGACTGGGTGCTCAGGTAGTGGTTGTCC | |
| | GGCAGCAGCACGGGGCCGTCGCCGATGGGGGTGTTCTCCTGGTA | |
| | GTCCTCGGCGAGCTGCACGCTGCCGTCCTCGATGTTGTCCCGGA | |
| | TCTTGAAGTTCACCTTGATGCCCTTCTTCTGCTTGTCGGCCATG | |
| | ATATAGACGTTGTGGCTGTTGTAGTTGTACTCCAGCTTGTGCCC | |
| | CAGGATGTTGCCGTCCTCCTTGAAGTCGATGCCCTTCAGCTCGA | |
| | TGCGGTTCACCAGGGTGTCGCCCTCGAACTTCACCTCGGCGCGG | |
| | GTCTTGTAGTTGCCGTCGTCCTTGAAGAAGATGGTGCGCTCCTG | |
| | GACGTAGCCTTCGGGCATGGCGGACTTGAAGAAGTCGTGCTGCT | |
| | TCATGTGGTCGGGGTAGCGGCTGAAGCACTGCACGCCGTAGCTC | |
| | AGGGTGGTCACGAGGGTGGGCCAGGGCACGGGCAGCTTGCCGGT | |
| | GGTGCAGATGAACTTCAGGGTCAGCTTGCCGTAGGTGGCATCGC | |
| | CCTCGCCCTCGCCGGACACGCTGAACTTGTGGCCGTTTACGTCG | |
| | CCGTCCAGCTCGACCAGGATGGGCACCACCCCGGTGAACAGCTC | |
| | CTCGCCCTTGCTCACCATGGTGGCGACCGGTGGGGAGAGAGGTC | |
| | GGTGATTCGGTCAACGAGGGAGCCGACTGCCGACGTGCGCTCCG | |
| | GAGGCTTGCAGAATGCGGAACACCGCGCGGGCAGGAACAGGGCC | |
| | CACACTACCGCCCCACACCCCGCCTCCCGCACCGCCCCTTCCCG | |
| | GCCGCTGCTCTCGGCGCGCCCTGCTGAGCAGCCGCTATTGGCCA | |
| | CAGCCCCATCGCGGTCGGCGCGCTGCCATTGTCCCTGGCGCTGT | |
| | CCGTCTGCGAGGGTACTAGTGAGACGTGCGGCTTCCGTTTGTCA | |
| | CGTCCGGCACGCCGCGAACCGCAAGGAACCTTCCCGACTTAGGG | |
| | GCGGAGCAGGAAGCGTCGCCGGGGGGCCCACAAGGGTAGCGGCG | |
| | AAGATCCGGGTGACGCTGCGAACGGACGTGAAGAATGTGCGAGA | |
| | CCCAGGGTCGGCGCCGCTGCGTTTCCCGGAACCACGCCCAGAGC | |
| | AGCCGCGTCCCTGCGCAAACCCAGGGCTGCCTTGGAAAAGGCGC | |
| | AACCCCAACCCCGTGGAAGCTCCAAAGGACTCAAAGAACCTCTG | |
| | GATGCTTTGACATACGATTTTTAATAAAACATGAGCATTTGAAT | |
| | AAAAACGACTTCCTCATACTGTAAACATCACGCATGCACATTAG | |
| | ACAATAATCCAGTAACGAAACGGCTTCAGTCGTAATCGCCCATA | |
| | TAGTTGGCTACAGAATGTTGGATAGAGAACTTAAGTACGCTAAG | |
| | GCGGCGTATTTTCTTAATATTTAGGGGTATTGCCGCAGTCATTA | |
| | CAGATACTCAGGAGTCAGATGCACCATGGTGTCTGTTTGAGGTT | |
| | GCTAGTGAACACAGTTGTGTCAGAAGCAAATGTAAGCAATAGAT | |
| | GGCTCTGCCCTGACTTTTATGCCCAGCCCTGGCTCCTGCCCTCC | |
| | CTGCTCCTGGGAGTAGATTGGCCAACCCTAGGGTGTGGCTCCAC | |
| | AGGGTGA | |
| Donor template | TACTCTTAATTCATTACATATTGTGCGGTCGAATTCAGGGAGCC | 355 |
| design 3 | GATAATGCGGTTACAATAATTCCTATACTTAAATATACAAAGAT | |
| (no HA) | TTAAAATTTCAAAAAATGGTTACCAGCATCGTTAGTGCGTATAC | |
| | ATCAAGAGGCACGTGCCCCGGAGACAGCAAGTAAGCTCTTTAAA | |
| | CATGCTTTGACATACGATTTTTAATAAAACATGAGCATTTGAAT | |
| | AAAAACGACTTCCTCATACTGTAAACATCACACGCATGCATTAG | |
| | ACAATAATCCAGTAACGAAACGGCTTCAGTCGTAATCGCCCATA | |
| | TAGTTGGCTACAGAATGTTGGATAGAGAACTTAAGTACGCTAAG | |
| | GCGGCGTATTTTCTTAATATTTAGGGGTATTGCCGCAGTCATTA | |
| | CAGATAACCGCCTATGCGGCCATGCCAGGATTATAGATAACTTT | |
| | TTAACATTAGCCGCAGAGGTGGGACTAGCACGTAATATCAGCAC | |
| | ATAACGTGTCAGTCAGGTCATCGACCTCGTCGGACTCCGGGTGC | |
| | GAGGTCGTGAAGCTGGAATACGAGTGAGGCCGCCGAGGACGTCA | |
| | GGGGGGTGTAAAGCTTCCATAGAGCCCACCGCATCCCCAGCATG | |
| | CCTGCTATTGTCTTCCCAATCCTCCCCCTTGCTGTCCTGCCCCA | |
| | CCCCACCCCCCAGAATAGAATGACACCTACTCAGACAATGCGAT | |
| | GCAATTTCCTCATTTTATTAGGAAAGGACAGTGGGAGTGGCACC | |
| | TTCCAGGGTCAAGGAAGGCACGGGGGAGGGGCAAACAACAGATG | |
| | GCTGGCAACTAGAAGGCACAGTCGAGGCTGATCAGCGGGTTTAA | |
| | ACGGGCCCTCTAGACTCGACGCGGCCGCTTTACTTGTACAGCTC | |
| | GTCCATGCCGAGAGTGATCCCGGCGGCGGTCACGAACTCCAGCA | |
| | GGACCATCTGATCGCGCTTCTCGTTGGGGTCTTTGCTCAGCCCC | |
| | GACTGGGTGCTCAGGTAGTGGTTGTCGGGCAGCAGCACGGGGCC | |
| | GTCGCCGATGGGGGTGTTCTGCTGGTAGTGGTCGGCGAGCTGCA | |
| | CGCTGCCGTCCTCGATGTTGTGGCGGATCTTGAAGTTCACCTTG | |
| | ATGCCGTTCTTCTGCTTGTCGGCCATGATATAGACGTTGTGGCT | |
| | GTTGTAGTTGTACTCCAGCTTGTGCCCCAGGATGTTGCCGTCCT | |
| | CCTTGAAGTCGATGCCCTTCAGCTCGATGCGGTTCACCAGGGTG | |
| | TCGCCCTCGAACTTCACCTCGGCGCGGGTCTTGTAGTTGCCGTC | |
| | GTCCTTGAAGAAGATGGTGCGCTCCTGGACGTAGCCTTCGGGCA | |
| | TGGCGGACTTGAAGAAGTCGTGCTGCTTCATGTGGTCGGGGTAG | |
| | CGGCTGAAGCACTGCACGCCGTAGGTCAGGGTGGTCACGAGGGT | |
| | GGGCCAGGGCACGGGCAGCTTGCCGGTGGTGCAGATGAACTTCA | |
| | GGGTCAGCTTGCCGTAGGTGGCATCGCCCTCCCCCTCGCCGGAC | |
| | ACGCTGAACTTGTGGCCGTTTACGTCGCCGTCCAGCTCGACCAG | |
| | GATGGGCACCACCCCGGTGAACAGCTCCTCGCCCTTGCTCACCA | |
| | TGGTGGCGACCGGTGGGGAGAGAGGTCGGTGATTCGGTCAACGA | |
| | GGGAGCCGACTGCCGACGTGCGCTCCGGAGGCTTGCAGAATCCC | |
| | GAACACCGCGCGGGCAGGAACAGGGCCCACACTACCGCCCCACA | |
| | CCCCGCCTCCCGCACCGCCCCTTCCCGGCCGCTGCTCTCGGCGC | |
| | GCCCTGCTGAGCAGCCGCTATTGGCCACAGCCCAGCGCGGTCGG | |
| | CGCGCTGCCATTGCTCCCTGGCGCTGTCCGTCTGCGAGGGTACT | |
| | AGTGAGACGTGCGGCTTCCGTTTGTCACGTCCGGCACGCCGCGA | |
| | ACCGCAAGGAACCTTCCCGACTTAGGGGCGGAGCAGGAAGCGTC | |
| | GCCGGGGGGCCCACAAGGGTAGCGGCGAAGATCCGGGTGACGCT | |
| | GCGAACGGACGTGAAGAATGTGCGAGACCCAGGGTCGGCGCCGC | |
| | TGCGTTTCCCGGAACCACGCCCAGAGCAGCCGCGTCCCTGCGCA | |
| | AACCCAGGGCTGCCTTGGAAAAGGCGCAACCCCAACCCCGTGGA | |
| | AGCTTGCGACCTGGAATCGGACAGCAGCGGGGAGTGTACGGCCC | |
| | CGAGTTCGTGACCGGGTATGCTTTCATTGTACCCCGGAACTTTA | |
| | AATCTATGAACAATCGCAACAAATTGTCCAAAGGCAACAATACG | |
| | ACACAGTTAGAGGCCATCGGCGCAGGTACACTCTATCCACGCCT | |
| | ATCAGAATGTCACCTGGTTAATGGTCAATTTAGGTGGCTGGAGG | |
| | CACATGTGAAGCAATATGGTCTAGGGAAAGATATCGGTTTACTT | |
| | AGATTTTATAGTTCCGGATCCAACTTAAATAATATAGGTATTAA | |
| | AGAGCAGTATCAAGAGGGTTTCTTCCCAAGGAATCTTGCGATTT | |
| | TCATACACAGCTTTAACAAATTTCACTAGACGCACCTTCATTTT | |
| | GTCGTCTCGTTGTATATGAGTCCGGGGTAAGAATTTTTTACCGT | |
| | ATTTAACATGATCAACGGGTACTAAAGCAATGTCATTTCTAAAC | |
| | ACAGTAGGTAAAGGACACGTCATCTTATTTTAAAGAATGTCAGA | |
| | AATCAGGGAGACTAGATCGATATTACGTGTTTT | |

Targeted integration experiments were conducted in primary CD4+ T cells with wild-type S. pyogenes ribonucleoprotein (RNP) targeted to the HBB locus. AAV6 was added at different multiplicities of infection (MOI) after nucleofection of 50 pmol of RNP. GFP fluorescence was measured 7 days after the experiment and showed that targeted integration frequency with the shorter homology arms was as efficient as when the longer homology arms were used (FIG. 2B). Assessment of targeted integration by digital droplet PCR (ddPCR) to either the 5′ or 3′ integration junction showed that (1) HA length did not affect targeted integration and (2) phenotypic assessment of targeted integration by GFP expression dramatically underestimated actual genomic targeted integration.

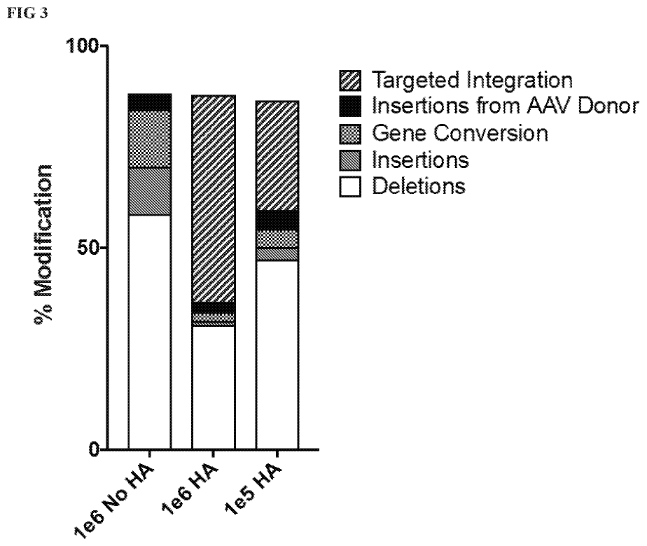

The genomic DNA from the cells that received the 177 nt HA donor (1e6 or 1e5 MOI) or no HA donor (1e6 MOI) was amplified with the 5′ and 3′ primers (P1 and P2), the PCR fragment was subcloned into a Topo Blunt Vector, and the resulting plasmids were Sanger sequenced. All high quality reads mapped one of the three expected PCR amplicons and the total number of reads were: 1e6 No HA—77 reads, 1e6 HA Donor—422 reads, 1e5 HA Donor—332 reads. The analysis allowed for the determination of on-target editing events at the HBB locus, including insertions, deletions, gene conversion from the highly homologous HBD gene, insertions from fragmented AAV donors, and targeted integration (FIG. 3A). To calculate targeted integration, the following formulas were used, taking into account the total number of reads from the 1^{st} Amplicon (AmpX), 2^{nd} Amplicon (AmpY), and 3^{rd} Amplicon (AmpZ). The results are summarized in Table 8 below.

$$Sequencing ( Overall ) = \frac{Average ( AmpY + AmpZ )}{AmpX + Average ( AmpY + AmpZ )} \times 100$$

$$Sequencing ( {5}^{′} ) = \frac{AmpY}{AmpX + AmpY} \times 100$$

$$Sequencing ( {3}^{′} ) = \frac{AmpZ}{AmpX + AmpZ} \times 100$$

TABLE 8
Comparison of Targeted Integration Frequency at
HBB locus Using Different Methods of Calculation.

| | Assay | % Integration |
|---|---|---|
| 1e6 MOI | GFP | 9.6% |
| | 5′ ddPCR | 70% |
| | 3′ ddPCR | 62% |
| | Sequencing | 51% |
| | (Overall) | |
| | Sequencing | 57% |
| | (5′ Junction) | |
| | Sequencing | 43.9% |
| | (3′ Junction) | |
| 1e5 MOI | GFP | 4.3% |
| | 5′ ddPCR | 21.9% |
| | 3′ ddPCR | 20% |
| | Sequencing | 27.2% |
| | (Overall) | |
| | Sequencing | 31.9% |
| | (5′ Junction) | |
| | Sequencing | 21.8% |
| | (3′ Junction) | |

The sequencing (overall) formula described above provided an estimate for the targeted integration taking into consideration reads from both the 2^{nd} amplicon (AmpY) and 3rd amplicon (AmpZ). When either the 2^{nd} amplicon (AmpY) or 3rd amplicon (AmpZ) was used alone to calculate targeted integration, the output was similar, showing that this method can be used with only 1 integrated priming site (either P1′ or P2′). The sequencing read-out matched the ddPCR analysis from either the 5′ or 3′ junction, indicating no PCR biases in the amplification, and that this method can be used to determine all on-target editing events.

### Example 2: Targeted Integration at HBB Locus in Adult Mobilized Peripheral Blood Human CD34+ Cells

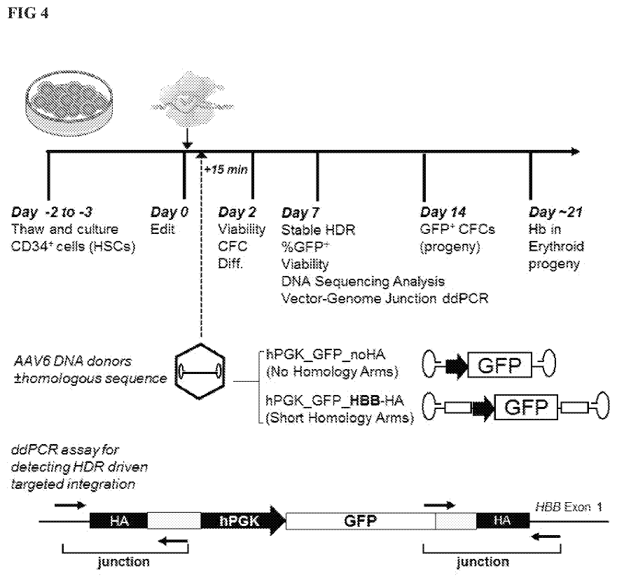

In this example, the goal was to determine the baseline level of targeted integration at the HBB locus in hematopoietic stem/progenitor cells, the population of cells which would be targeted clinically for gene correction or cDNA replacement for the treatment of b-hemoglobinopathies. Here, the donors described in Example 1 and depicted in FIG. 2A and Table 5, were used to deliver the PGK-GFP transgene expression cassette flanked by short homology arms (HA). The experimental schematic, timing and readouts for targeted integration are depicted in FIG. 4. Targeted integration experiments were conducted in human mobilized peripheral blood (mPB) CD34⁺ cells with wild-type S. pyogenes ribonucleoprotein (RNP) targeted to the HBB locus. Cells were cultured for 3 days in StemSpan-SFEM supplemented with human cytokines (SCF, TPO, FL, IL6) and dmPGE2. Cells were electroporated with the Maxcyte System and AAV6±HA (vector dose: 5×10⁴ vg/cell) was added to the cells 15-30 minutes after electroporation of the cells with 2.5 μM RNP (using HBB8 gRNA—targeting sequence CAGACUUCUCCACAGGAGUC). Two days after electroporation, CD34+ cells viability was assessed in the cells and cells were plated into Methocult to evaluate ex vivo hematopoietic differentiation potential and expression of GFP in their erythroid and myeloid progeny. On day 7 after electroporation, GFP fluorescence was evaluated by flow cytometly analysis in the viable CD34+ cell fraction. In addition, assessment of targeted integration was also analyzed by digital droplet PCR (ddPCR) to both the 5′ or 3′ integration junction, ddPCR analysis and Sanger sequencing analysis were performed as described in Example 1.

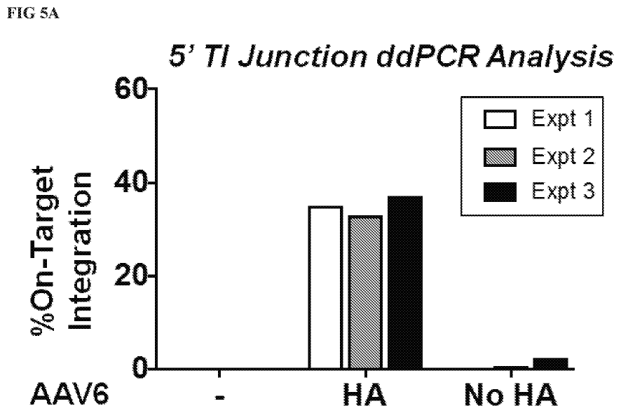

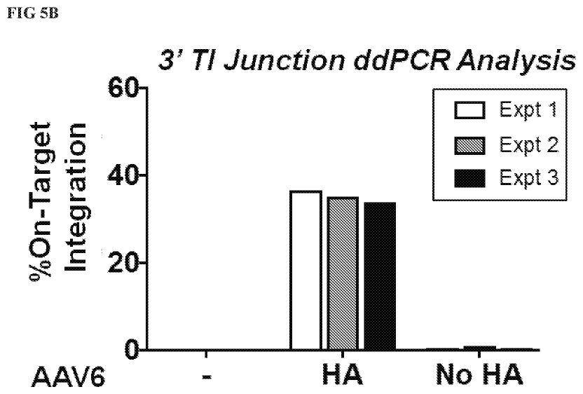

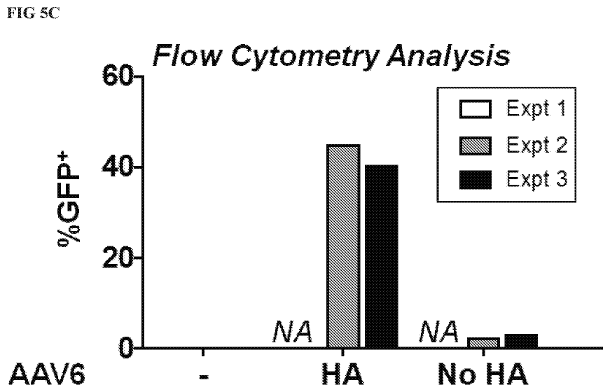

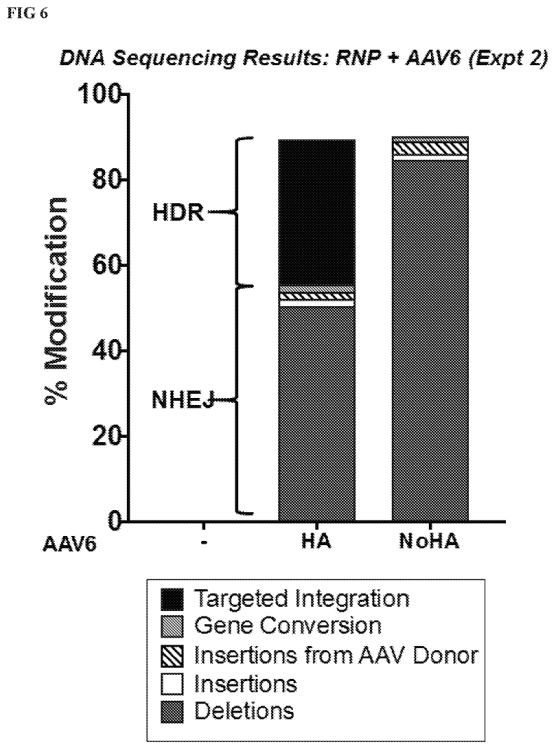

Three separate experiments were conducted and the day 7 targeted integration results are depicted in FIG. 5. Targeted integration as determined by 5′ and 3′ ddPCR analysis was ˜35% (FIG. 5A, 5B). Expression of the integration GFP transgene in CD34⁺ cells 7 days after electroporation was consistent with the ddPCR data, indicating that the integrated transgene was expressed (FIG. 5C). DNA sequencing analysis confirmed these results, with 35% HDR and 55% NHEJ detected in gDNA of CD34⁺ cells treated with RNP and AAV6 with HA (FIG. 6, total editing 90%). In contrast, for CD34⁺ cells treated with RNP and AAV6 without HA, no targeted integration was detected, and the only HDR observed was 1.7% gene conversion (that is gene conversion between HBB and HBD), while total editing frequency was the same (90%).

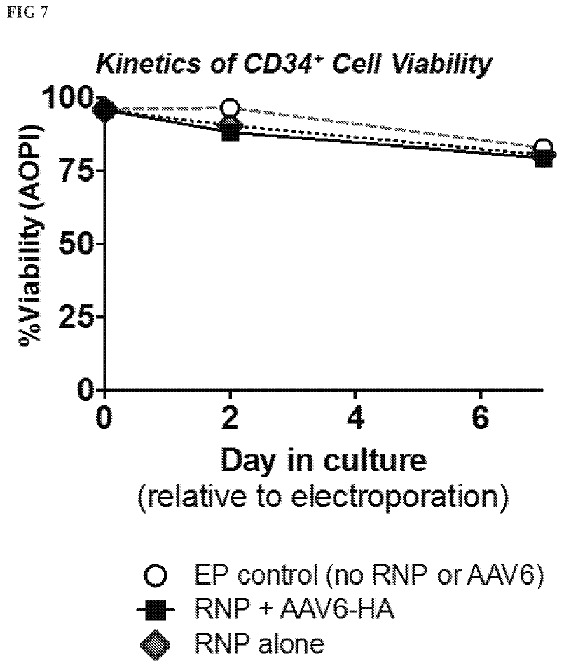

Importantly, between days 0 and 7 after electroporation there was no substantial difference in the viability (as determined by AOPI) of cells treated with RNP+ AAV or untreated (EP electroporation control) (FIG. 7). This indicates that the RNP and AAV6 combination is well-tolerated by CD34⁺ cells.

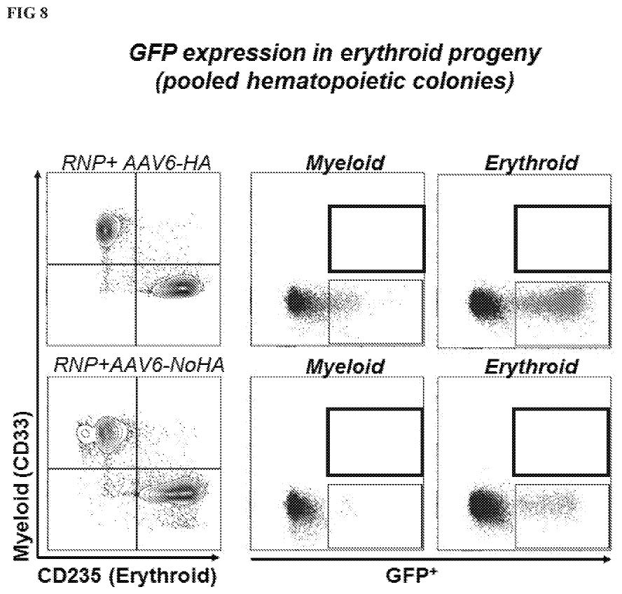

To determine whether the cells containing the targeted integration maintain differentiation potential, CD34⁺ cells on day 2 were plated into Methocult to evaluate ex vivo hematopoietic activity. On day 14 after plating CD34⁺ cells into Methocult, GFP⁺ colonies were scored by fluorescence microscopy. For the CD34⁺ cells treated with RNP with AAV6-HA and RNP with AAV6 with no HA, the percentages of GFP⁺ colonies were 32% and 2%, respectively. Pooled colonies were collected, pooled, immunostained with anti-human CD235 antibody (detecting Glycophorin A, erythroid specific cell surface antigen) and anti-human CD33 antibody (detected a myeloid specific cell surface antigen) and then analyzed by flow cytometry analysis. GFP expression was higher in the CD235⁺ erythroid vs.CD33⁺ myeloid cell fraction for progeny of cells treated with AAV6 (FIG. 8). This suggests that although the human PGK promoter is regulating transgene expression, higher expression occurs in the erythroid progeny, consistent with the integration of this gene into erythroid specific location (HBB gene). These data also show that integration is maintained in differentiated progeny of HDR-edited CD34⁺ cells.

### Sequences

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein.

Such equivalents are intended to be encompassed by the following claims.

### REFERENCES

Aliyu et al. Am J Hematol 83:63-70 (2008)
Angastiniotis & Modell Ann N Y Acad Sci 850:251-269 (1998)
Anders et al. Nature 513(7519):569-573 (2014)
Bae et al. Bioinformatics 30(10):1473-1475 (2014)
Bothmer et al. Nat Commun 8:13905 (2017)
Bouva Hematologica 91(1): 129-132 (2006)
Briner et al. Mol Cell 56(2):333-339 (2014)
Brousseau Am J Hematol 85(1):77-78 (2010)
Canvers et al. Nature 527(12):192-197 (2015)
Chang et al. Mol Ther Methods Clin Dev 4:137-148 (2017)
Chen et al. Nat Commun 8:14958 (2017)
Cong et al. Science 399(6121):819-823 (2013)
Comish-Bowden Nucleic Acids Res 13(9):3021-3030 (1985)
Davis & Maizels Proc Natl Acad Sci USA 111(10):E924-E932 (2014)
Fine et al. Sci Rep 5:10777 (2015)
Frit et al. DNA Repair (Amst.) 17:81-97 (2014)
Fu et al. Nat Biotechnol 32(3):279-284 (2014)
Guilinger et al. Nat Biotechnol 32(6):577-582 (2014)
Heigwer et al. Nat Methods 11(2):122-123 (2014)
Hinz et al. J Biol Chem 291(48):24851-24856 (2016)
Hsu et al. Nat Biotechnol 31(9):827-832 (2013)
lyama & Wilson DNA Repair (Amst.) 12(8):620-636 (2013)
Jiang et al. Nat Biotechnol 31(3):233-239 (2013)
Jinek et al. Science 337(6096):816-821 (2012)
Jinek et al. Science 343(6176): 1247997 (2014)
Kleinstiver et al. Nature 523(7561):481-485 (2015a)
Kleinstiver et al. Nat Biotechnol 33(12): 1293-1298 (2015b)
Kleinstiver et al. Nature 529(7587):490-495 (2016)
Komor et al. Nature 533(7603):420-424 (2016)
Lee et al. Nano Lett 12(12):6322-6327 (2012)
Lewis “Medical-Surgical Nursing: Assessment and Management of Clinical Problems” (2014)
Makarova et al. Nat Rev Microbiol 9(6):467-477 (2011)
Mali et al. Science 339(6121):823-826 (2013)
Nishimasu et al. Cell 156(5):935-949 (2014)
Nishimasu et al. Cell 162(5):1113-1126 (2015)
Ran et al. Cell 154(6):1380-1389 (2013)
Ran et al. Nature 520(7546):186-191 (2015)
Richardson et al. Nat Biotechnol 34(3):339-344 (2016)
Shmakov et al. Mol Cell 60:385-397 (2015)
Thein Hum Mol Genet 18(R2):R216-223 (2009)
Tsai et al. Nat Biotechnol 34(5):483 (2016)
Wang et al. Cell 153(4):910-918 (2013)
Xiao et al. Bioinformatics 30(8): 1180-1182 (2014)
Yamano et al. Cell 165(4):949-962 (2016)
Zetsche et al. Nat Biotechnol 33(2): 139-142 (2015a)
Zetsche et al. Cell 163(3):759-771 (2015b)

1. A genome editing system, comprising:
a ribonucleic acid (RNA) guided nuclease;
a guide RNA targeting a target nucleic acid of an HBB gene; and
an isolated nucleic acid for integration into the HBB gene, wherein:
(a) a first strand of the target nucleic acid comprises, from 5′ to 3′, P1--H1--X--H2--P2, wherein
P1 is a first priming site;
H1 is a first homology arm;
X is the cleavage site;
H2 is a second homology arm; and
P2 is a second priming site; and
(b) a first strand of the isolated nucleic acid comprises, from 5′ to 3′, A1--P2′--N--A2, or
A1--N--P1′-A2, wherein
A1 is a homology arm that is substantially identical to H1;
P2′ is a priming site that is substantially identical to P2;
N is a cargo;
P1′ is a priming site that is substantially identical to P1; and
A2 is a homology arm that is substantially identical to H2.

2. The genome editing system of claim 1, wherein the first strand of the isolated nucleic acid comprises, from 5′ to 3′, A1--P2′--N--P1′--A2.

3. The genome editing system of claim 1 or claim 2, furthering comprising S1 or S2,
wherein the first strand of the isolated nucleic acid comprises, from 5′ to 3′,
A1--S1--P2′--N--A2, or A1--N--P1′--S2--A2;
wherein S1 is a first stuffer, wherein S2 is a second stuffer, and wherein each of S1 and S2 comprise a random or heterologous sequence having a GC content of approximately 40%.

4. The genome editing system of claim 3, wherein the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site.

5. The genome editing system of claim 3 or claim 4, wherein the first stuffer has a sequence comprising at least 10 nucleotides of a sequence set forth in Table 2, and wherein the second stuffer has a sequence comprising at least 10 nucleotides of a sequence set forth in Table 2.

6. The genome editing system of any one of claims 3-5, wherein the first stuffer has a sequence that is not the same as the sequence of the second stuffer.

7. The genome editing system of any one of claims 3-6, wherein the first strand of the isolated nucleic acid comprises, from 5′ to 3′, A1--S1--P2′--N--P1′--S2--A2.

8. The genome editing system of claim 7, wherein A1+S1 and A2+S2 have sequences that are of approximately equal length.

9. The genome editing system of claim 8, wherein A1+S1 and A2+S2 have sequences that are of equal length.

10. The genome editing system of claim 7, wherein A1+S1 and H1+X+H2 have sequences that are of approximately equal length.

11. The genome editing system of claim 10, wherein A1+S1 and H1+X+H2 have sequences that are of equal length.

12. The genome editing system of claim 7, wherein A2+S2 and H1+X+H2 have sequences that are of approximately equal length.

13. The genome editing system of claim 12, wherein A2+S2 and H1+X+H2 have sequences that are of equal length.

14. The genome editing system of any one of claims 1-13, wherein A1 has a sequence that is at least 40 nucleotides in length, and A2 has a sequence that is at least 40 nucleotides in length.

15. The genome editing system of any one of claims 1-14, wherein A1 has a sequence that is identical to, or differs by no more than 1, 2, 3, 4, 5, 6, 7, 8, 9, 10, 15, 20, 25, or 30 nucleotides from a sequence of H1.

16. The genome editing system of any one of claims 1-15, wherein A2 has a sequence that is identical to, or differs by no more than 1, 2, 3, 4, 5, 6, 7, 8, 9, 10, 15, 20, 25, or 30 nucleotides from a sequence of H2.

17. The genome editing system of claim 7, wherein
A1+S1 have a sequence that is at least 40 nucleotides in length, and
A2+S2 have a sequence that is at least 40 nucleotides in length.

18. The genome editing system of any one of the previous claims, wherein N comprises an exon of a gene sequence, an intron of a gene sequence, a cDNA sequence, or a transcriptional regulatory element, a reverse complement of any of the foregoing or a portion of any of the foregoing.

19. The genome editing system of any one of claims 1-17, wherein N comprises a promoter sequence.

20. A composition comprising the genome editing system of any of claims 1-19 and, optionally, a pharmaceutically acceptable carrier.

21. A vector or plurality of vectors encoding the genome editing system of any one of claims 1-19.

22. The vector or plurality of vectors of claim 21, wherein the vector is a viral vector.

23. The vector of claim 21, wherein the vector is an AAV vector, a lentivirus, a naked DNA vector, or a lipid nanoparticle.

24. A composition comprising the genome editing system of any of claims 1-19, wherein the isolated nucleic acid is carried by a viral vector.

25. The composition of claim 24, wherein the viral vector is a parvoviral vector and the guide RNA and RNA guided nuclease are complexed with one another.

26. A method of altering a cell comprising contacting the cell with a genome editing system of any of claims 1-19, a composition of claims 20, 24 or 25, or a vector of claims 21-23.

27. A kit comprising a genome editing system of any of claims 1-19, a composition of claims 20, 24 or 25, or a vector of claims 21-23.

28. A genome editing system of any of claims 1-19, a composition of claims 20, 24 or 25, or a vector of claims 21-23 for use in therapy.

29. A method of altering a cell, comprising the steps of:
forming, in at least one allele of an HBB gene of the cell, at least one single- or double-strand break, wherein the at least one allele of the HBB gene comprises a first strand comprising: a first homology arm 5′ to the cleavage site, a first priming site either within the first homology arm or 5′ to the first homology arm, a second homology arm 3′ to the cleavage site, and a second priming site either within the second homology arm or 3′ to the second homology arm, and
recombining an exogenous oligonucleotide donor template with the at least one allele of an HBB gene by homologous recombination to produce an HBB allele, wherein a first strand of the exogenous oligonucleotide donor template comprises either:
i) a cargo, a priming site that is substantially identical to the second priming site either within or 5′ to the cargo, a first donor homology arm 5′ to the cargo, and a second donor homology arm 3′ to the cargo; or
ii) a cargo, a first donor homology arm 5′ to the cargo, a priming site that is substantially identical to the first priming site either within or 3′ to the cargo, and a second donor homology arm 3′ to the cargo,
wherein the altered HBB allele comprises a nucleotide sequence encoding a functional β-globin protein.

30. The method of claim 29, wherein the first strand of the exogenous oligonucleotide donor template comprises, from 5′ to 3′, the first donor homology arm, the priming site that is substantially identical to the second priming site, the cargo, the priming site that is substantially identical to the first priming site, and the second donor homology arm.

31. The method of claim 29 or claim 30, wherein the first strand of the exogenous oligonucleotide donor template further comprises a first stuffer or a second stuffer,
wherein the first stuffer and the second stuffer each comprise a random or
heterologous sequence having a GC content of approximately 40%; and
wherein the first strand of the exogenous oligonucleotide donor template comprises, from 5′ to 3′,
i) the first donor homology arm, the first stuffer, the priming site that is substantially identical to the second priming site, and the second donor homology arm: or
ii) the first donor homology arm, the cargo, the priming site that is substantially identical to the first priming site, the second stuffer, and the second donor homology arm.

32. The method of claim 31, wherein the first stuffer has a sequence having less than 50% sequence identity to any nucleic acid sequence within 500 base pairs of the cleavage site, and wherein the second stuffer has a sequence having less than 50% sequence identity to any nucleic acid sequence within 500 base pairs of the cleavage site.

33. The method of claim 31 or claim 32, wherein the first stuffer has a sequence comprising at least 10 nucleotides of a sequence set forth in Table 2, and wherein the second stuffer has a sequence comprising at least 10 nucleotides of a sequence set forth in Table 2.

34. The method of any one of claims 31-34, wherein the first stuffer has a sequence that is not the same as the sequence of the second stuffer.

35. The method of any one of claims 31-34, wherein the first strand of the exogenous oligonucleotide donor template comprises, from 5′ to 3′, the first donor homology arm, the first stuffer, the priming site that is substantially identical to the second priming site, the cargo, the priming site that is substantially identical to the first priming site, the second stuffer, and the second donor homology arm.

36. The method of claim 29, wherein the altered HBB allele comprises, from 5′ to 3′,
i) the first priming site, the first donor homology arm, the priming site that is substantially identical to the second priming site, the cargo, the second donor homology arm, and the second priming site; or
ii) the first priming site, the first donor homology arm, the cargo, the priming site that is substantially identical to the first priming site, the second donor homology arm, and the second priming site.

37. The method of claim 30, wherein the altered HBB allele comprises, from 5′ to 3′, the first priming site, the first donor homology arm, the priming site that is substantially identical to the second priming site, the cargo, the priming site that is substantially identical to the first priming site, the second donor homology arm, and the second priming site.

38. The method of claim 35, wherein the altered HBB allele comprises, from 5′ to 3′, the first priming site, the first donor homology arm, the first stuffer, the priming site that is substantially identical to the second priming site, the cargo, the priming site that is substantially identical to the first priming site, the second stuffer, the second donor homology arm, and the second priming site.

39. The method of any one of claims 29-38, wherein the step of forming the at least one single- or double-strand break comprises contacting the cell with an RNA-guided nuclease.

40. The method of claim 39, wherein the RNA-guided nuclease is a Class 2 Clustered Regularly Interspersed Repeat (CRISPR)-associated nuclease.

41. The method of claim 40, wherein the RNA-guided nuclease is selected from the group consisting of a wild-type Cas9, a Cas9 nickase, a wild-type Cpf1, and a Cpf1 nickase.

42. The method of any one of claims 39-41, wherein contacting the cell with the RNA-guided nuclease comprises introducing into the cell a ribonucleoprotein (RNP) complex comprising the RNA-guided nuclease and a guide RNA (gRNA).

43. The method of any one of claims 29-42, wherein the step of recombining the exogenous oligonucleotide donor template into the HBB allele by homologous recombination comprises introducing the exogenous oligonucleotide donor template into the cell.

44. The method of claim 42 or claim 43, wherein the step of introducing comprises electroporation of the cell in the presence of the RNP complex and/or the exogenous oligonucleotide donor template.

45. A population of cells made by the method of any of claims 29-44.


SEQUENCE LISTING (355 sequences)

SEQ ID NO: 1 - protein, 1345 aa, Streptococcus mutans
  misc_feature (10)..(21): N-terminal RuvC-like domain
  misc_feature (759)..(766): RuvC-like domain
  misc_feature (837)..(863): HNH-like domain
  misc_feature (982)..(989): RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  misc_feature (10)..(21): N-terminal RuvC-like domain
  misc_feature (760)..(767): RuvC-like domain
  misc_feature (844)..(870): HNH-like domain
  misc_feature (989)..(996): RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  misc_feature (10)..(21): N-terminal RuvC-like domain
  misc_feature (762)..(769): RuvC-like domain
  misc_feature (840)..(866): HNH-like domain
  misc_feature (985)..(992): RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - DNA, 3159 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10 - DNA, 3159 bp, Staphylococcus aureus
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - DNA, 3159 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 12 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 13 - DNA, 3249 bp, Neisseria meningitidis
  misc_feature (1)..(3249): Exemplary codon optimized Cas9
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 14 - protein, 859 aa, Artificial Sequence
  Synthetic Cas9 consensus sequence derived from Sm, Sp, St, and Li Cas9 sequences
  misc_feature (4)..(4): Xaa can be any naturally occurring amino acid
  misc_feature (8)..(18): N-terminal RuvC-like domain
  misc_feature (21)..(21): Xaa can be any naturally occurring amino acid
  misc_feature (23)..(23): Xaa can be any naturally occurring amino acid
  misc_feature (24)..(24): Xaa can be any naturally occurring amino acid
  misc_feature (26)..(26): Xaa can be any naturally occurring amino acid
  misc_feature (29)..(31): Each Xaa can independently be any naturally occurring amino acid
  misc_feature (33)..(33): Xaa can be any naturally occurring amino acid
  misc_feature (36)..(36): Xaa can be any naturally occurring amino acid
  misc_feature (45)..(45): Xaa can be any naturally occurring amino acid
  misc_feature (54)..(54): Xaa can be any naturally occurring amino acid
  misc_feature (63)..(63): Xaa can be any naturally occurring amino acid
  misc_feature (71)..(71): Xaa can be any naturally occurring amino acid
  misc_feature (75)..(75): Xaa can be any naturally occurring amino acid
  misc_feature (76)..(76): Xaa can be any naturally occurring amino acid
  misc_feature (78)..(80): Each Xaa can independently be any naturally occurring amino acid
  misc_feature (82)..(82): Xaa can be any naturally occurring amino acid
  misc_feature (84)..(84): Xaa can be any naturally occurring amino acid
  misc_feature (85)..(85): Xaa can be any naturally occurring amino acid
  misc_feature (89)..(89): Xaa can be any naturally occurring amino acid
  misc_feature (90)..(90): Xaa can be any naturally occurring amino acid
  misc_feature (98)..(98): Xaa can be any naturally occurring amino acid
  misc_feature (100)..(100): Xaa can be any naturally occurring amino acid
  misc_feature (106)..(106): Xaa can be any naturally occurring amino acid
  misc_feature (113)..(113): Xaa can be any naturally occurring amino acid
  misc_feature (116)..(116): Xaa can be any naturally occurring amino acid
  misc_feature (125)..(125): Xaa can be any naturally occurring amino acid
  misc_feature (126)..(126): Xaa can be any naturally occurring amino acid
  misc_feature (128)..(133): Each Xaa can independently be any naturally occurring amino acid
  misc_feature (135)..(135): Xaa can be any naturally occurring amino acid
  misc_feature (137)..(137): Xaa can be any naturally occurring amino acid
  misc_feature (139)..(147): Each Xaa can independently be any naturally occurring amino acid
  misc_feature (153)..(155): Each Xaa can independently be any naturally occurring amino acid
  misc_feature (157)..(157): Xaa can be any naturally occurring amino acid
  misc_feature (159)..(159): Xaa can be any naturally occurring amino acid
  misc_feature (161)..(161): Xaa can be any naturally occurring amino acid
  misc_feature (163)..(163): Xaa can be any naturally occurring amino acid
  misc_feature (166)..(168): Each Xaa can independently be any naturally occurring amino acid
  misc_feature (170)..(170): Xaa can be any naturally occurring amino acid
  misc_feature (171)..(171): Xaa can be any naturally occurring amino acid
  misc_feature (173)..(175): Each Xaa can independently be any naturally occurring amino acid
  misc_feature (177)..(177): Xaa can be any naturally occurring amino acid
  misc_feature (183)..(183): Xaa can be any naturally occurring amino acid
  misc_feature (185)..(187): Each Xaa can independently be any naturally occurring amino acid
  misc_feature (189)..(189): Xaa can be any naturally occurring amino acid
  misc_feature (192)..(195): Each Xaa can independently be any naturally occurring amino acid
  misc_feature (198)..(198): Xaa can be any naturally occurring amino acid
  misc_feature (199)..(199): Xaa can be any naturally occurring amino acid
  misc_feature (202)..(202): Xaa can be any naturally occurring amino acid
  misc_feature (206)..(206): Xaa can be any naturally occurring amino acid
  misc_feature (207)..(207): Xaa can be any naturally occurring amino acid
  misc_feature (210)..(210): Xaa can be any naturally occurring amino acid
  misc_feature (212)..(212): Xaa can be any naturally occurring amino acid
  misc_feature (213)..(213): Xaa can be any naturally occurring amino acid
  misc_feature (219)..(219): Xaa can be any naturally occurring amino acid
  misc_feature (220)..(220): Xaa can be any naturally occurring amino acid
  misc_feature (222)..(222): Xaa can be any naturally occurring amino acid
  misc_feature (224)..(224): Xaa can be any naturally occurring amino acid
  misc_feature (226)..(226): Xaa can be any naturally occurring amino acid
  misc_feature (236)..(236): Xaa can be any naturally occurring amino acid
  misc_feature (240)..(240): Xaa can be any naturally occurring amino acid
  misc_feature (241)..(241): Xaa can be any naturally occurring amino acid
  misc_feature (244)..(246): Each Xaa can independently be any naturally occurring amino acid
  misc_feature (248)..(248): Xaa can be any naturally occurring amino acid
  misc_feature (249)..(249): Xaa can be any naturally occurring amino acid
  misc_feature (250)..(250): Xaa can be any naturally occurring amino acid
  misc_feature (252)..(254): Each Xaa can independently be any naturally occurring amino acid
  misc_feature (256)..(256): Xaa can be any naturally occurring amino acid
  misc_feature (257)..(257): Xaa can be any naturally occurring amino acid
  misc_feature (268)..(268): Xaa can be any naturally occurring amino acid
  misc_feature (271)..(271): Xaa can be any naturally occurring amino acid
  misc_feature (273)..(273): Xaa can be any naturally occurring amino acid
  misc_feature (277)..(277): Xaa can be any naturally occurring amino acid
  misc_feature (280)..(280): Xaa can be any naturally occurring amino acid
  misc_feature (281)..(281): Xaa can be any naturally occurring amino acid
  misc_feature (283)..(283): Xaa can be any naturally occurring amino acid
  misc_feature (289)..(289): Xaa can be any naturally occurring amino acid
  misc_feature (290)..(290): Xaa can be any naturally occurring amino acid
  misc_feature (292)..(294): Each Xaa can independently be any naturally occurring amino acid
  misc_feature (301)..(301): Xaa can be any naturally occurring amino acid
  misc_feature (308)..(308): Xaa can be any naturally occurring amino acid
  misc_feature (319)..(322): Each Xaa can independently be any naturally occurring amino acid
  misc_feature (328)..(328): Xaa can be any naturally occurring amino acid
  misc_feature (329)..(329): Xaa can be any naturally occurring amino acid
  misc_feature (335)..(337): Each Xaa can independently be any naturally occurring amino acid
  misc_feature (346)..(346): Xaa can be any naturally occurring amino acid
  misc_feature (347)..(347): Xaa can be any naturally occurring amino acid
  misc_feature (356)..(361): Each Xaa can independently be any naturally occurring amino acid
  misc_feature (363)..(363): Xaa can be any naturally occurring amino acid
  misc_feature (370)..(373): Each Xaa can independently be any naturally occurring amino acid
  misc_feature (375)..(375): Xaa can be any naturally occurring amino acid
  misc_feature (376)..(376): Xaa can be any naturally occurring amino acid
  misc_feature (379)..(379): Xaa can be any naturally occurring amino acid
  misc_feature (386)..(390): Each Xaa can independently be any naturally occurring amino acid
  misc_feature (393)..(393): Xaa can be any naturally occurring amino acid
  misc_feature (395)..(395): Xaa can be any naturally occurring amino acid
  misc_feature (396)..(396): Xaa can be any naturally occurring amino acid
  misc_feature (398)..(398): Xaa can be any naturally occurring amino acid
  misc_feature (400)..(400): Xaa can be any naturally occurring amino acid
  misc_feature (403)..(403): Xaa can be any naturally occurring amino acid
  misc_feature (407)..(407): Xaa can be any naturally occurring amino acid
  misc_feature (410)..(410): Xaa can be any naturally occurring amino acid
  misc_feature (411)..(411): Xaa can be any naturally occurring amino acid
  misc_feature (413)..(416): Each Xaa can independently be any naturally occurring amino acid
  misc_feature (418)..(418): Xaa can be any naturally occurring amino acid
  misc_feature (422)..(422): Xaa can be any naturally occurring amino acid
  misc_feature (428)..(428): Xaa can be any naturally occurring amino acid
  misc_feature (431)..(431): Xaa can be any naturally occurring amino acid
  misc_feature (433)..(433): Xaa can be any naturally occurring amino acid
  misc_feature (437)..(439): Each Xaa can independently be any naturally occurring amino acid
  misc_feature (445)..(445): Xaa can be any naturally occurring amino acid
  misc_feature (451)..(451): Xaa can be any naturally occurring amino acid
  misc_feature (456)..(456): Xaa can be any naturally occurring amino acid
  misc_feature (459)..(459): Xaa can be any naturally occurring amino acid
  misc_feature (465)..(469): Each Xaa can independently be any naturally occurring amino acid
  misc_feature (481)..(481): Xaa can be any naturally occurring amino acid
  misc_feature (482)..(482): Xaa can be any naturally occurring amino acid
  misc_feature (484)..(484): Xaa can be any naturally occurring amino acid
  misc_feature (490)..(490): Xaa can be any naturally occurring amino acid
  misc_feature (494)..(494): Xaa can be any naturally occurring amino acid
  misc_feature (495)..(502): RuvC-like domain
  misc_feature (497)..(497): Xaa can be any naturally occurring amino acid
  misc_feature (506)..(506): Xaa can be any naturally occurring amino acid
  misc_feature (510)..(510): Xaa can be any naturally occurring amino acid
  misc_feature (513)..(513): Xaa can be any naturally occurring amino acid
  misc_feature (514)..(514): Xaa can be any naturally occurring amino acid
  misc_feature (517)..(517): Xaa can be any naturally occurring amino acid
  misc_feature (520)..(520): Xaa can be any naturally occurring amino acid
  misc_feature (525)..(525): Xaa can be any naturally occurring amino acid
  misc_feature (526)..(526): Xaa can be any naturally occurring amino acid
  misc_feature (529)..(529): Xaa can be any naturally occurring amino acid
  misc_feature (531)..(531): Xaa can be any naturally occurring amino acid
  misc_feature (532)..(532): Xaa can be any naturally occurring amino acid
  misc_feature (534)..(534): Xaa can be any naturally occurring amino acid
  misc_feature (542)..(542): Xaa can be any naturally occurring amino acid
  misc_feature (546)..(546): Xaa can be any naturally occurring amino acid
  misc_feature (547)..(547): Xaa can be any naturally occurring amino acid
  misc_feature (553)..(553): Xaa can be any naturally occurring amino acid
  misc_feature (555)..(575): HNH-like domain
  misc_feature (556)..(556): Xaa can be any naturally occurring amino acid
  misc_feature (560)..(560): Xaa can be any naturally occurring amino acid
  misc_feature (563)..(563): Xaa can be any naturally occurring amino acid
  misc_feature (565)..(565): Xaa can be any naturally occurring amino acid
  misc_feature (567)..(567): Xaa can be any naturally occurring amino acid
  misc_feature (579)..(579): Xaa can be any naturally occurring amino acid
  misc_feature (582)..(582): Xaa can be any naturally occurring amino acid
  misc_feature (583)..(583): Xaa can be any naturally occurring amino acid
  misc_feature (585)..(585): Xaa can be any naturally occurring amino acid
  misc_feature (588)..(588): Xaa can be any naturally occurring amino acid
  misc_feature (590)..(590): Xaa can be any naturally occurring amino acid
  misc_feature (592)..(592): Xaa can be any naturally occurring amino acid
  misc_feature (594)..(596): Each Xaa can independently be any naturally occurring amino acid
  misc_feature (610)..(610): Xaa can be any naturally occurring amino acid
  misc_feature (616)..(616): Xaa can be any naturally occurring amino acid
  misc_feature (628)..(628): Xaa can be any naturally occurring amino acid
  misc_feature (631)..(631): Xaa can be any naturally occurring amino acid
  misc_feature (633)..(633): Xaa can be any naturally occurring amino acid
  misc_feature (634)..(634): Xaa can be any naturally occurring amino acid
  misc_feature (636)..(636): Xaa can be any naturally occurring amino acid
  misc_feature (638)..(641): Each Xaa can independently be any naturally occurring amino acid
  misc_feature (643)..(645): Each Xaa can independently be any naturally occurring amino acid
  misc_feature (653)..(653): Xaa can be any naturally occurring amino acid
  misc_feature (657)..(657): Xaa can be any naturally occurring amino acid
  misc_feature (659)..(659): Xaa can be any naturally occurring amino acid
  misc_feature (660)..(660): Xaa can be any naturally occurring amino acid
  misc_feature (665)..(665): Xaa can be any naturally occurring amino acid
  misc_feature (666)..(666): Xaa can be any naturally occurring amino acid
  misc_feature (668)..(668): Xaa can be any naturally occurring amino acid
  misc_feature (669)..(669): Xaa can be any naturally occurring amino acid
  misc_feature (670)..(677): RuvC-like domain
  misc_feature (680)..(680): Xaa can be any naturally occurring amino acid
  misc_feature (681)..(681): Xaa can be any naturally occurring amino acid
  misc_feature (683)..(683): Xaa can be any naturally occurring amino acid
  misc_feature (686)..(686): Xaa can be any naturally occurring amino acid
  misc_feature (696)..(696): Xaa can be any naturally occurring amino acid
  misc_feature (697)..(697): Xaa can be any naturally occurring amino acid
  misc_feature (704)..(704): Xaa can be any naturally occurring amino acid
  misc_feature (708)..(708): Xaa can be any naturally occurring amino acid
  misc_feature (710)..(710): Xaa can be any naturally occurring amino acid
  misc_feature (711)..(711): Xaa can be any naturally occurring amino acid
  misc_feature (714)..(714): Xaa can be any naturally occurring amino acid
  misc_feature (717)..(720): Each Xaa can independently be any naturally occurring amino acid
  misc_feature (722)..(722): Xaa can be any naturally occurring amino acid
  misc_feature (725)..(725): Xaa can be any naturally occurring amino acid
  misc_feature (727)..(727): Xaa can be any naturally occurring amino acid
  misc_feature (733)..(735): Each Xaa can independently be any naturally occurring amino acid
  misc_feature (740)..(740): Xaa can be any naturally occurring amino acid
  misc_feature (742)..(742): Xaa can be any naturally occurring amino acid
  misc_feature (749)..(754): Each Xaa can independently be any naturally occurring amino acid
  misc_feature (758)..(761): Each Xaa can independently be any naturally occurring amino acid
  misc_feature (763)..(768): Each Xaa can independently be any naturally occurring amino acid
  misc_feature (771)..(771): Xaa can be any naturally occurring amino acid
  misc_feature (774)..(777): Each Xaa can independently be any naturally occurring amino acid
  misc_feature (782)..(782): Xaa can be any naturally occurring amino acid
  misc_feature (784)..(786): Each Xaa can independently be any naturally occurring amino acid
  misc_feature (788)..(788): Xaa can be any naturally occurring amino acid
  misc_feature (790)..(790): Xaa can be any naturally occurring amino acid
  misc_feature (795)..(795): Xaa can be any naturally occurring amino acid
  misc_feature (799)..(799): Xaa can be any naturally occurring amino acid
  misc_feature (801)..(801): Xaa can be any naturally occurring amino acid
  misc_feature (802)..(802): Xaa can be any naturally occurring amino acid
  misc_feature (804)..(804): Xaa can be any naturally occurring amino acid
  misc_feature (806)..(813): Each Xaa can independently be any naturally occurring amino acid
  misc_feature (815)..(818): Each Xaa can independently be any naturally occurring amino acid
  misc_feature (820)..(820): Xaa can be any naturally occurring amino acid
  misc_feature (823)..(827): Each Xaa can independently be any naturally occurring amino acid
  misc_feature (829)..(829): Xaa can be any naturally occurring amino acid
  misc_feature (830)..(830): Xaa can be any naturally occurring amino acid
  misc_feature (832)..(832): Xaa can be any naturally occurring amino acid
  misc_feature (835)..(837): Each Xaa can independently be any naturally occurring amino acid
  misc_feature (842)..(844): Each Xaa can independently be any naturally occurring amino acid
  misc_feature (846)..(846): Xaa can be any naturally occurring amino acid
  misc_feature (848)..(848): Xaa can be any naturally occurring amino acid
  misc_feature (851)..(851): Xaa can be any naturally occurring amino acid
  misc_feature (857)..(857): Xaa can be any naturally occurring amino acid
  misc_feature (857)..(857): Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 15 - protein, 8 aa, Artificial Sequence
  RuvC-like domain
  VARIANT (2)..(2): Xaa is Val or His
  VARIANT (3)..(3): Xaa is Ile, Leu, or Val
  VARIANT (5)..(5): Xaa is Met or Thr
IXXEXARE

SEQ ID NO: 16 - protein, 8 aa, Artificial Sequence
  RuvC-like domain
  VARIANT (3)..(3): Xaa is Ile, Leu, or Val
IVXEMARE

SEQ ID NO: 17 - protein, 8 aa, Artificial Sequence
  RuvC-like domain
  VARIANT (4)..(4): Xaa is His or Leu
  VARIANT (7)..(7): Xaa is Arg or Val
  VARIANT (8)..(8): Xaa is Glu or Val
HHAXDAXX

SEQ ID NO: 18 - protein, 8 aa, Artificial Sequence
  RuvC-like domain
HHAHDAYL

SEQ ID NO: 19 - protein, 30 aa, Artificial Sequence
  N-terminal RuvC-like domain
  VARIANT (2)..(2): Xaa is Lys or Pro
  VARIANT (4)..(4): Xaa is Val, Leu, Ile, or Phe
  VARIANT (5)..(5): Xaa is Gly, Ala, or Ser
  VARIANT (6)..(6): Xaa is Leu, Ile, Val, or Phe
  MOD_RES (7)..(26): N-terminal RuvC-like domain, each Xaa can be any amino acid or absent, region may encompass 5-20 residues
  VARIANT (29)..(29): Xaa is Asp, Glu, Asn, or Gln
KXYXXXXXXXXXXXXXXXXXXXXXXXTDXY

SEQ ID NO: 20 - protein, 12 aa, Artificial Sequence
  N-terminal RuvC-like domain
  VARIANT (2)..(2): Xaa is Ile, Val, Met, Leu, or Thr
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (6)..(6): Xaa is Ser, Tyr, Asn, or Phe
  VARIANT (7)..(7): Xaa is Val, Ile, Leu, Cys, Thr, or Phe
  VARIANT (9)..(9): Xaa is Trp, Phe, Val, Tyr, Ser, or Leu
  VARIANT (10)..(10): Xaa is Ala, Ser, Cys, Val, or Gly
  VARIANT (11)..(11): Xaa is Val, Ile, Leu, Ala, Met, or His
  VARIANT (12)..(12): Any amino acid or absent
DXGXXXXGXXXX

SEQ ID NO: 21 - protein, 12 aa, Artificial Sequence
  N-terminal RuvC-like domain
  VARIANT (2)..(2): Xaa is Ile, Val, Met, Leu, or Thr
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (7)..(7): Xaa is Val, Ile, Leu, Cys, Thr, or Phe
  VARIANT (9)..(9): Xaa is Trp, Phe, Val, Tyr, Ser, or Leu
  VARIANT (10)..(10): Xaa is Ala, Ser, Cys, Val, or Gly
  VARIANT (11)..(11): Xaa is Val, Ile, Leu, Ala, Met, or His
  VARIANT (12)..(12): Any amino acid or absent
DXGXXSXGXXXX

SEQ ID NO: 22 - protein, 12 aa, Artificial Sequence
  N-terminal RuvC-like domain
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (11)..(11): Xaa is Val, Ile, Leu, Ala, Met, or His
  VARIANT (12)..(12): Any amino acid or absent
DIGXXSVGWAXX

SEQ ID NO: 23 - protein, 12 aa, Artificial Sequence
  N-terminal RuvC-like domain
  MOD_RES (12)..(12): Any non-polar alkyl amino acid or a hydroxyl amino acid
DIGTNSVGWAVX

SEQ ID NO: 24 - protein, 73 aa, Artificial Sequence
  HNH-like domain
  VARIANT (8)..(8): Xaa is Lys or Arg
  VARIANT (12)..(12): Xaa is Val or Thr
  VARIANT (13)..(13): Xaa is Gly or Asp
  VARIANT (14)..(14): Xaa is Glu, Gln, or Asp
  VARIANT (15)..(15): Xaa is Glu or Asp
  VARIANT (19)..(19): Xaa is Asp, Asn, or His
  VARIANT (20)..(20): Xaa is Tyr, Arg, or Asn
  VARIANT (23)..(23): Xaa is Gln, Asp, or Asn
  MOD_RES (25)..(64): HNH-like domain, each Xaa can be any amino acid or absent, region may encompass 15-40 residues
  VARIANT (67)..(67): Xaa is Gly or Glu
  VARIANT (69)..(69): Xaa is Ser or Gly
  VARIANT (71)..(71): Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 25 - protein, 27 aa, Artificial Sequence
  HNH-like domain
  VARIANT (1)..(1): Xaa is Asp, Glu, Gln, or Asn
  VARIANT (2)..(2): Xaa is Leu, Ile, Arg, Gln, Val, Met, or Lys
  VARIANT (3)..(3): Xaa is Asp or Glu
  VARIANT (5)..(5): Xaa is Ile, Val, Thr, Ala, or Leu
  VARIANT (6)..(6): Xaa is Val, Tyr, Ile, Leu, Phe, or Trp
  VARIANT (8)..(8): Xaa is Gln, His, Arg, Lys, Tyr, Ile, Leu, Phe, or Trp
  VARIANT (9)..(9): Xaa is Ser, Ala, Asp, Thr, or Lys
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (13)..(13): Xaa is Asp, Ser, Asn, Arg, Leu, or Thr
  VARIANT (14)..(14): Xaa is Asp, Asn, or Ser
  VARIANT (15)..(15): Xaa is Ser, Ala, Thr, Gly, or Arg
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (19)..(19): Xaa is Lys, Leu, Arg, Met, Thr, or Phe
  VARIANT (20)..(20): Xaa is Val, Leu, Ile, Ala, or Thr
  VARIANT (21)..(21): Xaa is Leu, Ile, Val, or Ala
  VARIANT (22)..(22): Xaa is Thr, Val, Cys, Glu, Ser, or Ala
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XXXHXXPXXXXXXXXXXNXXXXXXXXN

SEQ ID NO: 26 - protein, 27 aa, Artificial Sequence
  HNH-like domain
  VARIANT (1)..(1): Xaa is Asp or Glu
  VARIANT (2)..(2): Xaa is Leu, Ile, Arg, Gln, Val, Met, or Lys
  VARIANT (3)..(3): Xaa is Asp or Glu
  VARIANT (5)..(5): Xaa is Ile, Val, Thr, Ala, or Leu
  VARIANT (6)..(6): Xaa is Val, Tyr, Ile, Leu, Phe, or Trp
  VARIANT (8)..(8): Xaa is Gln, His, Arg, Lys, Tyr, Ile, Leu, Phe, or Trp
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (22)..(22): Xaa is Thr, Val, Cys, Glu, Ser, or Ala
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XXXHXXPXSXXXDDSXXNKVLXXXXXN

SEQ ID NO: 27 - protein, 27 aa, Artificial Sequence
  HNH-like domain
  VARIANT (1)..(1): Xaa is Asp or Glu
  VARIANT (3)..(3): Xaa is Asp or Glu
  VARIANT (8)..(8): Xaa is Gln, His, Arg, Lys, Tyr, Ile, Leu, or Trp
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XVXHIVPXSXXXDDSXXNKVLTXXXXN

SEQ ID NO: 28 - protein, 27 aa, Artificial Sequence
  HNH-like domain
  VARIANT (2)..(2): Xaa is Ile or Val
  VARIANT (6)..(6): Xaa is Ile or Val
  VARIANT (9)..(9): Xaa is Ala or Ser
  VARIANT (11)..(11): Xaa is Ile or Leu
  VARIANT (12)..(12): Xaa is Lys or Thr
  VARIANT (14)..(14): Xaa is Asp or Asn
  VARIANT (19)..(19): Xaa is Arg, Lys, or Leu
  VARIANT (22)..(22): Xaa is Thr or Val
  VARIANT (23)..(23): Xaa is Ser or Arg
  VARIANT (25)..(25): Xaa is Lys, Asp, or Ala
  VARIANT (26)..(26): Xaa is Glu, Lys, Gly, or Asn
DXDHIXPQXFXXDXSIDNXVLXXSXXN

SEQ ID NO: 29 - RNA, 116 nt, Artificial Sequence
  gRNA
  misc_feature (1)..(20): a, c, u, g, unknown or other
  misc_feature (1)..(20): targeting region
  misc_feature (21)..(42): first complementarity domain
  misc_feature (43)..(46): linking domain
  misc_feature (47)..(70): second complementarity domain
nnnnnnnnnnnnnnnnnnnnguuuuagagcuaugcuguuuuggaaacaaaacagcauagcaaguuaaaauaaggcuaguccguuaucaacuugaaaaaguggcaccgagucggugc

SEQ ID NO: 30 - RNA, 116 nt, Artificial Sequence
  gRNA
  misc_feature (1)..(20): a, c, u, g, unknown or other
  misc_feature (1)..(20): targeting region
  misc_feature (21)..(42): first complementarity domain
  misc_feature (43)..(46): linking domain
  misc_feature (47)..(70): second complementarity domain
nnnnnnnnnnnnnnnnnnnnguauuagagcuaugcuguauuggaaacaauacagcauagcaaguuaauauaaggcuaguccguuaucaacuugaaaaaguggcaccgagucggugc

SEQ ID NO: 31 - RNA, 96 nt, Artificial Sequence
  gRNA
  misc_feature (1)..(20): a, c, u, g, unknown or other
  misc_feature (1)..(20): targeting domain
  misc_feature (21)..(32): first complementarity domain
  misc_feature (33)..(36): linking domain
  misc_feature (37)..(50): second complementarity domain
  misc_feature (51)..(62): proximal domain
nnnnnnnnnnnnnnnnnnnnguuuaagagcuagaaauagcaaguuuaaauaaggcuaguccguuaucaacuugaaaaaguggcaccgagucggugc

SEQ ID NO: 32 - RNA, 47 nt, Artificial Sequence
  gRNA proximal and tail domains derived from S. pyogenes
aaggcuaguccguuaucaacuugaaaaaguggcaccgagucggugcu

SEQ ID NO: 33 - RNA, 49 nt, Artificial Sequence
  gRNA proximal and tail domains
aaggcuaguccguuaucaacuugaaaaaguggcaccgagucgguggugc

SEQ ID NO: 34 - RNA, 51 nt, Artificial Sequence
  gRNA proximal and tail domains
aaggcuaguccguuaucaacuugaaaaaguggcaccgagucggugcggauc

SEQ ID NO: 35 - RNA, 31 nt, Artificial Sequence
  gRNA proximal and tail domains
aaggcuaguccguuaucaacuugaaaaagug

SEQ ID NO: 36 - RNA, 18 nt, Artificial Sequence
  gRNA proximal and tail domains
aaggcuaguccguuauca

SEQ ID NO: 37 - RNA, 12 nt, Artificial Sequence
  gRNA proximal and tail domains
aaggcuaguccg

SEQ ID NO: 38 - RNA, 102 nt, Artificial Sequence
  Unimolecular gRNA derived from S. aureus
  misc_feature (1)..(20): n is a, c, g, or u
  misc_feature (1)..(20): Targeting domain
nnnnnnnnnnnnnnnnnnnnguuuuaguacucuggaaacagaaucuacuaaaacaaggcaaaaugccguguuuaucucgucaacuuguuggcgagauuuuuu

SEQ ID NO: 39 - RNA, 42 nt, Artificial
  Modular gRNA derived from S. pyogenes
  misc_feature (1)..(20): a, c, u, g, unknown or other
  misc_feature (1)..(20): Targeting domain
  misc_feature (21)..(42): First complementarity domain
nnnnnnnnnnnnnnnnnnnnguuuuagagcuaugcuguuuug

SEQ ID NO: 40 - RNA, 85 nt, Artificial Sequence
  Modular gRNA derived from S. pyogenes
  misc_feature (1)..(9): 5′ extension domain
  misc_feature (10)..(33): Second complementarity domain
  misc_feature (34)..(45): Proximal domain
  misc_feature (46)..(85): Tail domain
ggaaccauucaaaacagcauagcaaguuaaaauaaggcuaguccguuaucaacuugaaaaaguggcaccgagucggugcuuuuuu

SEQ ID NO: 41 - RNA, 62 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
nnnnnnnnnnnnnnnnnnnnguuuuagagcuagaaauagcaaguuaaaauaaggcuaguccg

SEQ ID NO: 42 - RNA, 102 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
  misc_feature (63)..(102): Tail domain
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 43 - RNA, 75 nt, Artificial Sequence
  Unimolecular gRNA derived from S. pyogenes
  misc_feature (1)..(20): Targeting domain
  misc_feature (1)..(20): a, c, u, g, unknown or other
  misc_feature (21)..(36): First complementarity domain
  misc_feature (37)..(40): Linking domain
  misc_feature (41)..(58): Second complementarity domain
  misc_feature (59)..(70): Proximal domain
  misc_feature (71)..(75): Tail domain
nnnnnnnnnnnnnnnnnnnnguuuuagagcuaugcugaaaagcauagcaaguuaaaauaaggcuaguccguuauc

SEQ ID NO: 44 - RNA, 87 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (43)..(46): Linking domain
  misc_feature (57)..(70): Second complementarity domain
  misc_feature (71)..(82): Proximal domain
  misc_feature (83)..(87): Tail domain
nnnnnnnnnnnnnnnnnnnnguuuuagagcuaugcuguuuuggaaacaaaacagcauagcaaguuaaaauaaggcuaguccguuauc

SEQ ID NO: 45 - RNA, 42 nt, Artificial Sequence
  Modular gRNA derived from S. thermophilus
  misc_feature (1)..(20): a, c, u, g, unknown or other
  misc_feature (1)..(20): Targeting domain
  misc_feature (21)..(42): First complementarity domain
nnnnnnnnnnnnnnnnnnnnguuuuagagcuguguuguuucg

SEQ ID NO: 46 - RNA, 78 nt, Artificial Sequence
  Modular gRNA derived from S. thermophilus
  misc_feature (1)..(3): 5′ extension domain
  misc_feature (4)..(27): Second complementarity domain
  misc_feature (28)..(40): Proximal domain
  misc_feature (41)..(78): Tail domain
gggcgaaacaacacagcgaguuaaaauaaggcuuaguccguacucaacuugaaaagguggcaccgauucgguguuuuu

SEQ ID NO: 47 - RNA, 85 nt, Artificial Sequence
  Modular gRNA derived from S. pyogenes
gaaccauucaaaacagcauagcaaguuaaaauaaggcuaguccguuaucaacuugaaaaaguggcaccgagucggugcuuuuuuu

SEQ ID NO: 48 - RNA, 96 nt, Artificial Sequence
  gRNA from S. pyogenes
  misc_feature (1)..(20): n is a, c, g, or u
  misc_feature (1)..(20): Targeting domain
nnnnnnnnnnnnnnnnnnnnguuuuagagcuagaaauagcaaguuaaaauaaggcuaguccguuaucaacuugaaaaaguggcaccgagucggugc

SEQ ID NO: 49 - RNA, 96 nt, Artificial Sequence
  gRNA
  misc_feature (1)..(20): n is a, c, g, or u
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
nnnnnnnnnnnnnnnnnnnnguauuagagcuagaaauagcaaguuaauauaaggcuaguccguuaucaacuugaaaaaguggcaccgagucggugc

SEQ ID NO: 50 - RNA, 104 nt, Artificial Sequence
  gRNA
  misc_feature (1)..(20): n is a, c, g, or u
  misc_feature (1)..(20): Targeting domain
  misc_feature (21)..(36): First complementarity domain
  misc_feature (37)..(40): Linking domain
  misc_feature (41)..(58): Second complementarity domain
nnnnnnnnnnnnnnnnnnnnguuuuagagcuaugcugaaaagcauagcaaguuaaaauaaggcuaguccguuaucaacuugaaaaaguggcaccgagucggugc

SEQ ID NO: 51 - RNA, 106 nt, Artificial Sequence
  gRNA
  misc_feature (1)..(20): n is a, c, g, or u
  misc_feature (1)..(20): Targeting domain
  misc_feature (21)..(37): First complementarity domain
  misc_feature (38)..(41): Linking domain
  misc_feature (42)..(60): Second complementarity domain
nnnnnnnnnnnnnnnnnnnnguuuuagagcuaugcuggaaacagcauagcaaguuaaaauaaggcuaguccguuaucaacuugaaaaaguggcaccgagucggugc

SEQ ID NO: 52 - protein, 12 aa, Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, S. mutans
DIGTNSVGWAVV

SEQ ID NO: 55 - protein, 12 aa, S. pyogenes
DIGTNSVGWAVI

SEQ ID NO: 56 - protein, 12 aa, L. innocua
DIGTNSVGWAVL

SEQ ID NO: 57 - protein, 12 aa, Flavobacterium branchiophilum FL-15
DLGTNSIGWAVV

SEQ ID NO: 58 - protein, 11 aa, Pedobacter glucosidilyticus
DLGTNSIGWAI

SEQ ID NO: 59 - protein, 12 aa, Bacteroides fragilis, NCTC 9343
DLGTNSIGWALV

SEQ ID NO: 60 - protein, 12 aa, Fusobacterium nucleatum
DIGTNSVGWCVT

SEQ ID NO: 61 - protein, 12 aa, Acidaminococcus sp. D21
DIGTNSVGYAVT

SEQ ID NO: 62 - protein, 12 aa, Coprococcus catus GD-7
DMGTGSLGWAVT

SEQ ID NO: 63 - protein, 12 aa, Oenococcus kitaharae DSM 17330
DIGTSSVGWAAI

SEQ ID NO: 64 - protein, 12 aa, Catenibacterium mitsuokai DSM 15897
DLGTGSVGWAVV

SEQ ID NO: 65 - protein, 12 aa, Mycoplasma gallisepticum str. F
DLGVGSVGWAIV

SEQ ID NO: 66 - protein, 12 aa, Mycoplasma ovipneumoniae SC01
DLGIASIGWAII

SEQ ID NO: 67 - protein, 12 aa, Mycoplasma canis PG 14
DLGIASVGWAIV

SEQ ID NO: 68 - protein, 12 aa, Mycoplasma synoviae 53
DLGVASVGWSIV

SEQ ID NO: 69 - protein, 12 aa, Eubacterium rectale
DIGIASVGWAIL

SEQ ID NO: 70 - protein, 12 aa, Enterococcus faecalis TX0012
DLGISSVGWSVI

SEQ ID NO: 71 - protein, 12 aa, Ilyobacter polytropus DSM 2926
DIGIASVGWSVI

SEQ ID NO: 72 - protein, 12 aa, Ruminococcus albus 8
DVGIGSIGWAVI

SEQ ID NO: 73 - protein, 12 aa, Elusimicrobium minutum Pei191
DLGVGSIGFAIV

SEQ ID NO: 74 - protein, 12 aa, Akkermansia muciniphila
DIGYASIGWAVI

SEQ ID NO: 75 - protein, 12 aa, Prevotella ruminicola
DTGTNSLGWAIV

SEQ ID NO: 76 - protein, 12 aa, Cand. Puniceispirillum marinum
DLGTNSIGWCLL

SEQ ID NO: 77 - protein, 12 aa, Rhodospirillum rubrum
DIGTDSLGWAVF

SEQ ID NO: 78 - protein, 12 aa, Lactobacillus rhamnosus GG
DIGSNSIGFAVV

SEQ ID NO: 79 - protein, 12 aa, Sphaerochaeta globus str. Buddy
DLGVGSIGVAVA

SEQ ID NO: 80 - protein, 12 aa, Rhodopseudomonas palustris
DLGIASCGWGVV

SEQ ID NO: 81 - protein, 12 aa, Mycoplasma mobile 163K
DLGIASVGWCLT

SEQ ID NO: 82 - protein, 12 aa, Streptococcus thermophilus LMD-9
DIGIGSVGVGIL

SEQ ID NO: 83 - protein, 12 aa, Staphylococcus lugdunensis M23590
DIGITSVGYGLI

SEQ ID NO: 84 - protein, 12 aa, Eubacterium dolichum DSM 3991
DIGITSVGFGII

SEQ ID NO: 85 - protein, 12 aa, Lactobacillus coryniformis KCTC 3535
DVGITSTGYAVL

SEQ ID NO: 86 - protein, 12 aa, Nitratifractor salsuginis DSM 16511
DLGITSFGYAIL

SEQ ID NO: 87 - protein, 12 aa, Bifidobacterium bifidum S17
DIGNASVGWSAF

SEQ ID NO: 88 - protein, 12 aa, Lactobacillus gasseri
DVGTNSCGWVAM

SEQ ID NO: 89 - protein, 12 aa, Acidothermus cellulolyticus 11B
DVGERSIGLAAV

SEQ ID NO: 90 - protein, 12 aa, Bifidobacterium longum DJO10A
DVGLNSVGLAAV

SEQ ID NO: 91 - protein, 12 aa, Bifidobacterium dentium
DVGLMSVGLAAI

SEQ ID NO: 92 - protein, 12 aa, Corynebacterium diphtheriae
DVGTFSVGLAAI

SEQ ID NO: 93 - protein, 12 aa, Staphylococcus pseudintermedius ED99
DIGTGSVGYACM

SEQ ID NO: 94 - protein, 12 aa, Capnocytophaga ochracea
DLGTTSIGFAHI

SEQ ID NO: 95 - protein, 12 aa, Prevotella denticola
DLGTNSIGSSVR

SEQ ID NO: 96 - protein, 12 aa, Ralstonia solanacearum
DIGTNSIGWAVI

SEQ ID NO: 97 - protein, 12 aa, Pasteurella multocida str. Pm70
DLGIASVGWAVV

SEQ ID NO: 98 - protein, 12 aa, Comamonas granuli
DIGIASVGWAVL

SEQ ID NO: 99 - protein, 12 aa, Helicobacter mustelae 12198
DIGIASIGWAVI

SEQ ID NO: 100 - protein, 12 aa, Agathobacter rectalis
DIGIASVGWAII

SEQ ID NO: 101 - protein, 12 aa, Clostridium cellulolyticum H10
DVGIASVGWAVI

SEQ ID NO: 102 - protein, 11 aa, Methylophilus sp. OH31
DIGIASVGWAL

SEQ ID NO: 103 - protein, 12 aa, Neisseria meningitidis
DIGIASVGWAMV

SEQ ID NO: 104 - protein, 12 aa, Clostridium perfringens
DIGITSVGWAVI

SEQ ID NO: 105 - protein, 12 aa, Wolinella succinogenes DSM 1740
DLGISSLGWAIV

SEQ ID NO: 106 - protein, 12 aa, Azospirillum sp. B510
DLGTNSIGWGLL

SEQ ID NO: 107 - protein, 12 aa, Verminephrobacter eiseniae
DLGSTSLGWAIF

SEQ ID NO: 108 - protein, 12 aa, Campylobacter jejuni, NCTC 11168
DIGISSIGWAFS

SEQ ID NO: 109 - protein, 12 aa, Parvibaculum lavamentivorans DS-1
DIGTTSIGFSVI

SEQ ID NO: 110 - protein, 12 aa, Dinoroseobacter shibae DFL 12
DIGTSSIGWWLY

SEQ ID NO: 111 - protein, 12 aa, Nitrobacter hamburgensis X14
DLGSNSLGWFVT

SEQ ID NO: 112 - protein, 12 aa, Bradyrhizobium sp. BTAi1
DLGANSLGWFVV

SEQ ID NO: 113 - protein, 15 aa, Bacillus cereus
DIGLRIGITSCGWSI

SEQ ID NO: 114 - protein, 12 aa, Sutterella wadsworthensis
DMGAKYTGVFYA

SEQ ID NO: 115 - protein, 12 aa, Wolinella succinogenes DSM 1740
DLGGKNTGFFSF

SEQ ID NO: 116 - protein, 12 aa, Francisella tularensis
DLGVKNTGVFSA

SEQ ID NO: 117 - protein, 12 aa, Gamma proteobacterium HTCC5015
DLGAKFTGVALY

SEQ ID NO: 118 - protein, 12 aa, Legionella pneumophila str. Paris
DLGGKFTGVCLS

SEQ ID NO: 119 - protein, 12 aa, Parasutterella excrementihominis
DLGGTYTGTFIT

SEQ ID NO: 120 - protein, 12 aa, S. thermophilus
DIGTNSVGWAVT

SEQ ID NO: 121 - protein, 12 aa, Eubacterium yurii
DVGTNSVGWAVT

SEQ ID NO: 122 - protein, 12 aa, Butyrivibrio hungatei
DMGTNSVGWAVT

SEQ ID NO: 123 - protein, 12 aa, Solobacterium moorei F0204
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Coprococcus catus GD-7
DIDHIYPQSKTMDDSLNNRVLVKKNYN

SEQ ID NO: 126 - protein, 27 aa, Peptoniphilus duerdenii
DQDHIYPKSKIYDDSLENRVLVKKNLN

SEQ ID NO: 127 - protein, 27 aa, Catenibacterium mitsuokai DSM 15897
QIDHIVPQSLVKDDSFDNRVLVVPSEN

SEQ ID NO: 128 - protein, 27 aa, S. mutans
DIDHIIPQAFIKDNSIDNRVLTSSKEN

SEQ ID NO: 129 - protein, 27 aa, S. thermophilus
DIDHIIPQAFLKDNSIDNKVLVSSASN

SEQ ID NO: 130 - protein, 27 aa, Oenococcus kitaharae DSM 17330
DIDHIIPQAYTKDNSLDNRVLVSNITN

SEQ ID NO: 131 - protein, 27 aa, L. inocua
DIDHIVPQSFITDNSIDNLVLTSSAGN

SEQ ID NO: 132 - protein, 27 aa, S. pyogenes
DVDHIVPQSFLKDDSIDNKVLTRSDKN

SEQ ID NO: 133 - protein, 27 aa, Acidaminococcus sp. D21
NIDHIYPQSMVKDDSLDNKVLVQSEIN

SEQ ID NO: 134 - protein, 27 aa, Lactobacillus rhamnosus GG
DIDHILPQSLIKDDSLDNRVLVNATIN

SEQ ID NO: 135 - protein, 27 aa, Lactobacillus gasseri
DIDHILPQSFIKDDSLENRVLVKKAVN

SEQ ID NO: 136 - protein, 27 aa, Staphylococcus pseudintermedius ED99
EVDHIFPRSFIKDDSIDNKVLVIKKMN

SEQ ID NO: 137 - protein, 27 aa, Olsenella uli
EVDHIIPRSYIKDDSFENKVLVYREEN

SEQ ID NO: 138 - protein, 27 aa, Bifidobacterium bifidum S17
DIDHIIPQAVTQNDSIDNRVLVARAEN

SEQ ID NO: 139 - protein, 27 aa, Mycoplasma gallisepticum str. F
EIDHIIPYSISFDDSSSNKLLVLAESN

SEQ ID NO: 140 - protein, 27 aa, Mycoplasma canis PG 14
EIDHIIPYSLCFDDSSANKVLVHKQSN

SEQ ID NO: 141 - protein, 27 aa, Ilyobacter polytropus DSM 2926
DIDHIIPYSRSMDDSYSNKVLVLSGEN

SEQ ID NO: 142 - protein, 27 aa, Uncultured Termite group 1 bacterium
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Clostridium cellulolyticum H10
QIDHIYPYSRSMDDSYMNKVLVLTDEN

SEQ ID NO: 145 - protein, 27 aa, Clostridium perfringens
EIDHIIPFSRSFDDSLSNKILVLGSEN

SEQ ID NO: 146 - protein, 27 aa, N. meningitides
EIDHALPFSRTWDDSFNNKVLVLGSEN

SEQ ID NO: 147 - protein, 27 aa, Pasteurella multocida str. Pm70
EIDHALPFSRTWDDSFNNKVLVLASEN

SEQ ID NO: 148 - protein, 27 aa, Enterococcus faecalis TX0012
EIDHIIPISISLDDSINNKVLVLSKAN

SEQ ID NO: 149 - protein, 27 aa, Eubacterium dolichum DSM 3991
EVDHIIPISISLDDSITNKVLVTHREN

SEQ ID NO: 150 - protein, 27 aa, Acidovorax ebreus
QVDHALPYSRSYDDSKNNKVLVLTHEN

SEQ ID NO: 151 - protein, 27 aa, Streptococcus thermophilus LMD-9
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Staphylococcus lugdunensis M23590
EVDHIIPRSVSFDNSYHNKVLVKQSEN

SEQ ID NO: 154 - protein, 27 aa, Roseburia intestinalis
DIDHILPYSITFDDSFRNKVLVTSQEN

SEQ ID NO: 155 - protein, 27 aa, Wolinella succinogenes DSM 1740
EIDHILPRSRSADDSFANKVLCLARAN

SEQ ID NO: 156 - protein, 27 aa, Cand. Puniceispirillum marinum
EIEHLLPFSLTLDDSMANKTVCFRQAN

SEQ ID NO: 157 - protein, 27 aa, Azospirillum sp. B510
DIDHILPFSVSLDDSAANKVVCLREAN

SEQ ID NO: 158 - protein, 27 aa, Bradyrhizobium sp. BTAi1
DIDHLIPFSISWDDSAANKVVCMRYAN

SEQ ID NO: 159 - protein, 27 aa, Nitrobacter hamburgensis X14
DIDHILPVAMTLDDSPANKIICMRYAN

SEQ ID NO: 160 - protein, 27 aa, Dinoroseobacter shibae
DVDHILPYSRTLDDSFPNRTLCLREAN

SEQ ID NO: 161 - protein, 27 aa, Verminephrobacter eiseniae
EIEHILPFSRTLDDSLNNRTVAMRRAN

SEQ ID NO: 162 - protein, 27 aa, Lactobacillus coryniformis KCTC 3535
EVDHIIPYSISWDDSYTNKVLTSAKCN

SEQ ID NO: 163 - protein, 27 aa, Rhodopseudomonas palustris
QVDHILPWSRFGDDSYLNKTLCTARSN

SEQ ID NO: 164 - protein, 27 aa, Ralstonia syzygii R24
QVDHILPFSKTLDDSFANKVLAQHDAN

SEQ ID NO: 165 - protein, 27 aa, Helicobacter mustelae 12198
QIDHAFPLSRSLDDSQSNKVLCLTSSN

SEQ ID NO: 166 - protein, 27 aa, Mycoplasma mobile 163K
DIDHIVPRSISFDDSFSNLVIVNKLDN

SEQ ID NO: 167 - protein, 27 aa, Mycoplasma ovipneumoniae SC01
EIEHIIPYSMSYDNSQANKILTEKAEN

SEQ ID NO: 168 - protein, 27 aa, Mycoplasma synoviae 53
EIDHVIPYSKSADDSWFNKLLVKKSTN

SEQ ID NO: 169 - protein, 27 aa, Aminomonas paucivorans DSM 12260
EMDHILPYSRSLDNGWHNRVLVHGKDN

SEQ ID NO: 170 - protein, 27 aa, Ruminococcus albus 8
EVDHIVPYSLILDNTINNKALVYAEEN

SEQ ID NO: 171 - protein, 27 aa, Fibrobacter succinogenes
EIEHVIPQSLYFDDSFSNKVICEAEVN

SEQ ID NO: 172 - protein, 27 aa, Bacteroides fragilis, NCTC 9343
DIEHIIPQARLFDDSFSNKTLEARSVN

SEQ ID NO: 173 - protein, 27 aa, Capnocytophaga sputigena
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Parvibaculum lavamentivorans DS-1
EVEHIWPRSRSFDNSPRNKTLCRKDVN

SEQ ID NO: 176 - protein, 27 aa, Bacillus cereus
IVNHIIPYNRSFDDTYHNRVLTLTETK

SEQ ID NO: 177 - protein, 27 aa, Prevotella micans
DMEHTIPKSISFDNSDQNLTLCESYYN

SEQ ID NO: 178 - protein, 27 aa, Prevotella ruminicola
DIEHTIPRSAGGDSTKMNLTLCSSRFN

SEQ ID NO: 179 - protein, 27 aa, Flavobacterium columnare
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Elusimicrobium minutum Pei191
DIEHLFPIAESEDNGRNNLVISHSACN

SEQ ID NO: 182 - protein, 27 aa, Sphaerochaeta globus str. Buddy
DVDHIFPRDDTADNSYGNKVVAHRQCN

SEQ ID NO: 183 - protein, 27 aa, Nitratifractor salsuginis DSM 16511
DIEHIVPQSLGGLSTDYNTIVTLKSVN

SEQ ID NO: 184 - protein, 27 aa, Acidothermus cellulolyticus 11B
ELDHIVPRTDGGSNRHENLAITCGACN

SEQ ID NO: 185 - protein, 28 aa, Bifidobacterium longum DJO10A
EMDHIVPRKGVGSTNTRTNFAAVCAECN

SEQ ID NO: 186 - protein, 28 aa, Bifidobacterium dentium
EMDHIVPRKGVGSTNTRVNLAAACAACN

SEQ ID NO: 187 - protein, 28 aa, Corynebacterium diphtheriae
EMDHIVPRAGQGSTNTRENLVAVCHRCN

SEQ ID NO: 188 - protein, 33 aa, Sutterella wadsworthensis
EIDHILPRSLIKDARGIVFNAEPNLIYASSRGN

SEQ ID NO: 189 - protein, 33 aa, Gamma proteobacterium HTCC5015
EIDHIIPRSLTGRTKKTVFNSEANLIYCSSKGN

SEQ ID NO: 190 - protein, 33 aa, Parasutterella excrementihominis
EIDHIIPRSLTLKKSESIYNSEVNLIFVSAQGN

SEQ ID NO: 191 - protein, 33 aa, Legionella pneumophila str. Paris
EIDHIYPRSLSKKHFGVIFNSEVNLIYCSSQGN

SEQ ID NO: 192 - protein, 33 aa, Wolinella succinogenes DSM 1740
EIDHILPRSHTLKIYGTVFNPEGNLIYVHQKCN

SEQ ID NO: 193 - protein, 30 aa, Francisella tularensis
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Solobacterium moorei F0204
DIDHIYPRSKIKDDSITNRVLVEKDIN

SEQ ID NO: 196 - protein, 28 aa, Veillonella atypica ACS-134-V-Col7a
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN

SEQ ID NO: 199 - DNA, 5 bp, Streptococcus thermophilus
  misc_feature (1)..(1): Any nucleotide (e.g., A, G, C, or T)
  misc_feature (4)..(4): Any nucleotide (e.g., A, G, C, or T)
nggng

SEQ ID NO: 200 - DNA, 7 bp, Streptococcus thermophilus
  misc_feature (1)..(1): Any nucleotide (e.g., A, G, C, or T)
  misc_feature (2)..(2): Any nucleotide (e.g., A, G, C, or T)
  misc_feature (7)..(7): A or T
nnagaaw

SEQ ID NO: 201 - DNA, 4 bp, Streptococcus mutans
  misc_feature (1)..(1): Any nucleotide (e.g., A, G, C, or T)
  misc_feature (4)..(4): A or G
naar

SEQ ID NO: 202 - DNA, 5 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
nngrr

SEQ ID NO: 203 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
  misc_feature (6)..(6): Any nucleotide (e.g., A, G, C, or T)
nngrrn

SEQ ID NO: 204 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
nngrrt

SEQ ID NO: 205 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
  misc_feature (6)..(6): A, G, or C
nngrrv

SEQ ID NO: 206-250 - skipped (45 entries)

SEQ ID NO: 251 - DNA, 2000 bp, Artificial Sequence
  Exemplary stuffer sequence
tcaatagcccagtcggttttgttagatacattttatcgaatctgtaaagatattttataataagataatatcagcgcctagctgcggaattccactcagagaatacctctcctgaatatcagccttagtggcgttatacgatatttcacactctcaaaatcccgagtcagactatacccgcgcatgtttagtaaaggttgattctgagatctcgagtccaaaaaagatacccactactttaaagatttgcattcagttgttccatcggcctgggtagtaaagggggtatgctcgctccgagtcgatggaactgtaaatgttagccctgatacgcggaacatatcagtaacaatctttacctaatatggagtgggattaagcttcatagaggatatgaaacgctcgtagtatggcttcctacataagtagaattattagcaactaagatattaccactgcccaataaaagagattccacttagattcataggtagtcccaacaatcatgtctgaatactaaattgatcaattggactatgtcaaaattattttgaagaagtaatcatcaacttaggcgctttttagtgttaagagcgcgttattgccaaccgggctaaacctgtgtaactcttcaatattgtatataattataggcagaataagctatgagtgcattatgagataaacatagatttttgtccactcgaaatatttgaatttcttgatcctgggctagttcagccataagttttcactaatagttaggactaccaattacactacattcagttgctgaaattcacatcactgccgcaatatttatgaagctattattgcattaagacttaggagataaatacgaagttgatatatttttcagaatcagcgaaaagaccccctattgacattacgaattcgagtttaacgagcacataaatcaaacactacgaggttaccaagattgtatcttacattaatgctatccagccagccgtcatgtttaactggatagtcataattaatatccaatgatcgtttcacgtagctgcatatcgaggaagttgtataattgaaaacccacacattagaatgcatggtgcatcgctagggtttatcttatcttgctcgtgccaagagtgtagaaagccacatattgatacggaagctgcctaggaggttggtatatgttgattgtgctcaccatctcccttcctaatctcctagtgttaagtccaatcagtgggctggctctggttaaaagtaatatacacgctagatctctctactataatacaggctaagcctacgcgctttcaatgcactgattaccaacttagctacggccagccccatttaatgaattatctcagatgaattcagacattattctctacaaggacactttagagtgtcctgcggaggcataattattatctaagatggggtaagtccgatggaagacacagatacatcggactattcctattagccgagagtcaaccgttagaactcggaaaaagacatcgaagccggtaacctacgcactataaatttccgcagagacatatgtaaagttttattagaactggtatcttgattacgattcttaactctcatacgccggtccggaatttgtgactcgagaaaatgtaatgacatgctccaattgatttcaaaattagatttaaggtcagcgaactatgtttattcaaccgtttacaacgctattatgcgcgatggatggggccttgtatctagaaaccgaataataacatacctgttaaatggcaaacttagattattgcgattaattctcacttcagagggttatcgtgccgaattcctgactttggaataataaagttgatattgaggtgcaatatcaactacactggtttaacctttaaacacatggagtcaagttttcgctatgccagccggttatgcagctaggattaatattagagctcttttctaattcgtcctaataatctcttcac

SEQ ID NO: 252 - DNA, 2000 bp, Artificial Sequence
  Exemplary stuffer sequence
aaaacgtactacgtccactaatatagtgctcagggcctttaaagttatgaacaggaatacggcgatgacgatagagatgtacaactcagtgcgaaccccagtgtatgtacaaaaagttactaattcactttactgttttgaggatgtacctgccaaaaagattcagattatcaaagtcagatctttatatgacggaacgcgcaaaggatcctattaggatgcgcctcaaaaagccatctaaaaagttcatgtattgagcttattagtaaaggtatcaacaaaaatgattccaccttatataaataagcttgatcccattaattgaataataaagaccgagtaatcacttttatgcatgtaacaaaaatcccgtttgcggctatgctacaacggtcatcccatagaatattatcatcgtacaagcccaagacccgatgctcaacattagagccaaataacgtgcacactcctaatatgagatgactgccgcttttaacaccagatctgttagttaggccacgcacttccaagtttatctagagtgcatgtctttatatatgttggtcccctgtaatgacttataatatttccttcgactgtgttgaacatctgtaacaataaagactaaagctctgggtatataaggttgcagtggtaccttattaggtccattatcgcagaatactgcggatggacaatcttgccaatttaattgactatctattagtttgcacaatataacgattcgtcttggacaaatttggcgagtgagccccttactcgctcaaaatgttacaattgccgagctcggagttgaatgattagttacatattatagaacacaatgcagatgtagttagacaagatgtgttgatgaatgtcaagtctgactggagtaaaggaacaagagcacccacctacgtatattgcgcattttaaatgtagcctcgactctaacacgtgcgacgtgagtcataattgtgcatgttattagatctatggaatgttgtttttttaattatcaaacgtacgtcaaaccgccaaactccgtgtgccatagagtatactcctgaagttcgaaattaggccataaagtctttcttgctggttgtgaaatgaaggggtgtttcataatttaactttgactgcttctgttgggacgacgtacccgttcgtttgtttgtcctactatttagtatcttaaaacagtccatttaccgttaatgttcttaacccttaaagatacaaacttagctctgtaatcaacttcaagacgtctttgacagaacgtctaagacccagatctgtgttagccaactcgtattcaatttcgtaccggtggacttcggcccctcacactgccattagttgatgctgaactttgtatttgctgggtaggatatataacgattttgcagatgtgtgtgctaagtatattgtcttagtgacggtccagcatataaaacacctacacaagaaggttattcttaatggttgattgaatattattaaattgttgcttttactttttcctcctacaaattgtcatgagctcaaatttgttgacctaaggtattaatattgtatcctacacggattgtgaacggtagggtcgtaacaatcgtactttacggcttaaaaattgtaagcaccttgccaggtagatgaaaacttaaaggatagaagtatagtaactcacatgcttgcggcagcatcgtagggcagaggtgtgatcttggtgattgaaattaaggggtaggatgatcggccgcatatatcggctactaggattagatagatgcaacgctttactttaatcaagtgacgtccgtataagtaagacatctaatggctgtatttttgtatacaagtataaggaaccggggagtctttatagcgacgcgtaattatatattccaaatcagttaagtggcgtcggttacgaaactaaagagagtgttcaagacgcaatgaagaatcgtgagcgtaattgttcgcgc

SEQ ID NO: 253 - DNA, 2000 bp, Artificial Sequence
  Exemplary stuffer sequence
aaccctcgtgtccggtaaaacacgcttcgaatacaaaagattatataggtacggaaggctgggaatctttcttcgatggaactgagattatattccactgtaaccttattatgactatagatttccaacatacggatagattaataccgactgtagattccatacttgaactatgaagccgtacgagtacccatactataactaagactatgacacgtgtgaattcgtgtttatcatagtgcaaactcttgctattccacatgggagtttagaactcagctgttcctatacaattagcactacaaacccactaatatggatagcatgataccatctgaggaggatttggtgttaccatgttgtaatctaagaagtttcacaaaatcaacgttagataaacggcaatatacgcgcactaataatgaaccccaagatatcagttgaaaaattttcgatctcctctttaaattaacaaatattgcagagtaagtaccgaaattgtgacacaagtgccgtttgcccgtctttttcacagcctataaagttcagatctatatgggctcccacttaaccttcagatagataacaagttactggaagtgattctatcataatacaatcaactataacacatccaatgatatatctcgagaaagtcgtagtctagagctccttctattatccggtcttacctaaatagttatatttagttgcccatttaaaattggataggaggaggggtgctcatgatttaaaaaccaactgtgcatgcggttctttgatgtggatccaccttgcaaagcgctaaagataaaagtagtcactacaggaattcaacttccgtcgttgtcagctggcgcgggaacccatcttgtgtaaaaaactgtataaccagacacgtggactcgaccgagaaacagtcagaacctgtcacaagaaataatcttgattaaaggctttcacggcaaacggacctcttccctgctgaagtgtacgattgaatatccacatcgaaggtcaattaccctcatcttttacatggtcataagacaataatctcctatttggattaaaatccgcgcacgaaagataagagtggaatcgattgcattatcgagtttttaagccccatacccgacagatgtgtaaaaagtgtagtggtaatggcgtcaccaagacctatgcttctcataataataggacgtatgccctagctactgctaacggtcgctcttacaatactagctaaaagaaacaaatttgaaaagttatgtaggaagtcattggcggtgaaaaagtgagaaaaaaggtccccggagactgtgctttcatgttatcaaagtacatgccgagtgaagagtttgttttgatcaacttttattatctggagtcattatacgatattgccatggttccttggctgtccaaccaggggtcttttacaccagataatcttctactacactacacctcaggtacgattctttcgttatcaatcgactacaagattatagtgtctctaaggcgtgatgtaggttttccctcaatgacaaagactttacagcaatccggttcaatacgagaattaagtgtgcgagtaacagcaaagtaaaatctaacagaaaggagactcagaaaacaacctattgaggactgtaatatcaactcagcattattgtttactttaaaatctaataatcgtttcgaggatatgagcacggtatcctaacatcaagacaaataccacatcatctaaatacaactggttgcaatgagtcgaatcgcgaacaaataaagcaactataagcacgataaaccactgttatgggaatgataaacagtcttatgacgtggtctatctgtcgtaggtggtaaagccttctgaagatcactatccagttctggcctcaagaaccatttagacagccttttctaaacatgatcgttgctataaggaccggggacacctagacaaactcacggaagggataacttacatc

SEQ ID NO: 254 - DNA, 2000 bp, Artificial Sequence
  Exemplary stuffer sequence
actgcttatataggaggtacaaacagatacaatccttagttaactagagagaatgctttttttcgaccgacacgcttataacttcactgggcatggtcaccatatttaggtaaaacaaactgctgcgctatatgtcgtacacatcctgagtgtaccaatatgtaggtggaaggcaagttcaatgagacgtcagttaccaagcaaatttacattctagcagttataaatgtattatgacgcagttcttgtggtgagcgatcatttacattaaaactttattcaagagcgtatattagcatatattttccggagagtgcactacgggccgaaatttaggctggaactccgcaaattggttacgaccctgtatacatagttcttattattaagtaaaatgtgtgaataaaacctacacgacgcgtcatatacgtaaaagtttatctcttgtagtaatcaactaaattaacttactactatctggtcgtccgtatgaccctgtgagcagattattttcgactcgacatctatgaattctacggcacgaaaagttggtaacttgtactgggttaaacaatgtgtattcgggagtctgcggaagaacgtttttaatgtaacttcctttgcaaaccaaaatttggtctattcaaactgacactagcgtaatctataccgcatgagatcctgacatgatcctatatctatgcgcataggtactcgcaccaataagtgggtcgtagaatttcacgtaactcaatgttgtctcctttcattttttgttaattcgagaaaactacaaaaatagttagtaaaatgctcaaggagtcaggtgctacctgtggaatacatctatgtccaatggaacttgctccctcggatgtgcgatttcgttgttcagttgggcctttaaggaatacagcaactccaactctttgattttaggtaagtatttgattcgcggaaagtacagtgtataatctgttatttgccaagacgtcatcgaaatcgagtgtatcgagatcagaccatcgcgctatcgcaagatatgaagagcatagacagatcacgatgccaatcagtgtcgatggtgcgaagacgcagcccctgtgatcaaatcgtccgtttctcgatttactagcggaaaacaaaaacgaagcggtgaataccctgcgagctaatgtctttacccggttatacgagctgataactcggaaaatgctaatatcgaggctgcgcacttaaaaaaatactttaataatattaataagcatagctgtatcataacttaaaattctactgtatgatttagaatctaacagtgttaacgatctacagaccgcactaagatgaagacggactaatctcctccctaattttccttgttgattagcaaagggagatccttttgttatttgaggtttacgagaaagatgtaagagtcgaaataattacgtaaacctcatagtcgtcacctagagcaactataacatgaaccactcgccttggttaaatataaaataacttcttctctgtaacattgttgcacacaagcgagcgacaaaatttcacaacatttgttgcgtagataatattactgcatcatttttgcgtcagagtgaatgtcacttatataactaggaaaaattagtaggatagctcttgcggttgagagtaatgtcgactgaatcgaccgccatagatggtagagggagtgattcaaatagattaatgtatgcgctccatctataaggacggacaaggatcaatgttcccttatacttagctaacaggaccctctccgaaggtctgataatgcactcatataagcatcgatgcgtcctgagtagaaaaatctttacaaacttttaatagataagttatcttggaggtgctatctattcaaatctctgaacagatctgcggcatgataatgtctttgtaccggtgtgaataatgtgagtcagacgtctgtgcgaagtgggaaccgaaatcttttaatcatt

SEQ ID NO: 255 - DNA, 2000 bp, Artificial Sequence
  Exemplary stuffer sequence
gattcggtcgcgttccataatcgaacccttaagcccatcttccagctgttaacgttatgtaccatcttacctcaatgtcagcgatctatgaggttcatgtttttggtggattaaaaaacttctttatagtggtttagacagaacgtttagcgctgcgctcgaagtgtcttatctaacggaggactaaaattacctggtcactccttagacttttcgtagtacttaattgccggacatccgttgggctacaccagcaagaacacaaagtggtatgtgtgaagctagactgacctcatgattcgtactacattataagaatcaagcttcccggatttgtgttctgagatattaccacgtacatttttaagggggttcttgacatcgtaacgctaaggctgattaaagaggagggtgctatgcagagtttattggtgtttcatcaatgtatcacacaaaattagctactataggaagtagctttggtgcgagcagggggcggtatggttaagaaagctatggtaagaaaggcccaggtgatactacgtgtaaggttgtgaagagccacaagagccaagttttgatattcgacttcctccgaatctacagcttatcgagggttaaacgttacgcatattacgagattacatgatagcttctcagttctagcacatttatgagaccctttgaatggtgtcaataaataggaggtccccatatgacaagtagaatactaactataagagatttgtaacgctggataccatttgcagaggattggcccaaagaatgattgcccaacgcttatattgtcagaccttgcattagaagaataacgcagaatacgactgcagtttgatataattttggctctgggttgccttagtatcattactaatagacttgtggtctatatccatttgtttaatggaatagactgggtaaaacacacctcttccaggctgtagttcttcatgttgtaaggatccgtcatggcgtgcaaactaggggaggtattttttgctaattgcggtaacggctccagttgggatatcgtcaatatgtgccactcggccctttctctgagacgctaagatttccgtaaggtatagcgataagagtctctaatgccagaggaattgttaccgcgagcaagattcatgtctatatataaaatatcatccactttgaattactggttggaatcatcgttcgcgttataacaaaaaaccttttaattatgttaccacagatctcgaagtcccttttgaggcagaagtttaaatataagctctaattgtcgcatctaacgggtatatcgtctcaacggtaggtcaaaaacatttgttaacttcagactgtacattcgcatttaactcgccatgtaaaccgcaatacatctcgtgcctatctctcctagtaacgtattatcgctgggtgaaagcgcaactaagtaataagtgaatgtcattcacaatacctaactctatccgacgcgtaagagcgacccagcagtttaatgacatgataaatcaaattctatgcaaggcagtacttgctttgtggacgatagcgattttccaccgtattgcgaagtcagttatgctgaaattttattccattcgcataacaccaaggcttactcttaggaaaaaatgtaataccgattttggtatgaagtatgttacagtacagaatgaaatgcccggcggcgtggtcaaactgtttcctgaggttcatatagggaaaggtcatccctcagaattggccccgtaatcgcaaagcctacgggagctttcttaagtccaaccggtaaagccaaatctcaattcatatgaggaaatgtttgaccgataaagaatagattgtcgaactaacagtcacagagaaaatacgagtagcatcacctaaacaaagcaggtaataaaatagactaatggagatcatcgtatcggcttatgacctgcgtccatttaaaggcaatgaatacattaccgactaga

SEQ ID NO: 256 - DNA, 2000 bp, Artificial Sequence
  Exemplary stuffer sequence
agttatgaggttcacttctcatataacactatcaacaatgatcatctcttgcgaaacaagcgccctacacagcttcaatggaaccaagagccataatgaggtaagggacggctagttactaataaaggaatcgattttacaaacactaaatgaaaaacttgcgctggttgcaatgctataaaaaaatgaaatgcaaaccagtgaagatcccgatcaaccgttcgctgatttttattgatgctgtacgttgtgttagtttaatgatatataggccatctccaggttacttaggacgccaaaattactattttgaagctcaaccgtggtataatagctacaataattaattgatgcctgcaggtcgtatctcgaacgattgtacgcattacctatgatatgaacagaatctgtatcccatacttaaaatcttgaccttgtaaagatttcgcatacgcattaagaaatttcgttctacccgcacggattgtccaagtatatctggccattcacagaagttactaatcttcatctctaagtttaaggccgacaaagggtccaaaacctgcgtaggttacaacgcagcttacactcagtgactaaccaacgctcagtagggtaactggacttgttctcgctattcagctggtactgtaatgatcaacttagaacggccctatggctaagcaaggagtacgcaatgttttagaatacgtgtttgctcacacaggtagtagtttaatataccccctgacaagatatgttaacatagatgaagtttggtattacttatagccagactattcttcaacatatacactgggttttaggagtgtgcaatttataaggacagttatattcctacaatcgttgtatgatccttttgggtttggtagaactacgtttgggccgcgcctttggtcaaccacggactttctgtctagatgccaattcctacaagcttagtcctatcaatttagtagagaacaaattttgtcatcactgaattgtcgtcttactatcggatcattctccgctaattataggattattagtaacgcgtatataggagcgattaatgactcatcaatgaatagcatcactaggtgtattatatgaacctctctctattctattaactgcccactgtgggtaatttgagttatacctgaccggtccctcggatccttaatcctttgatgtcgataggtaactgaagtgtaagatcctgatatatgaagccggtaaggagacggagattttatattagtgttcttggatactgtgctagaaggttctactctaactcaaacaggttataaagtaggaaggaaaaagttgatagtggtaaactaattatgagttggcttgcttattccaagttagcgaggttttcatgacgtaagtctgataaggtttgctggaagctgaaaagttttacaaaaacgttgttttagaatggtttgtccccgaaaatcgaacctggcatagccctcaggagacgaacaagcccaggcaaaccgggggtttctcgcttattgctataatcacctctagtgttgtagaagcaattacggtggggaggcgtcaatgtggcctgagttccgttgaggacttttcacgtgtaggacccattaatagaggagatatatgtctttcagctgcggaattcataatagtggaaagaagaaaagggattactagattaatattactcatcccagacttaagttgaaagctacatcttcacacccaggaaaccggaccgcctttgttcaggtctaagtagtctggaacagaaccgtatcaactgccccaattcataggtgttagcgtgacagcgatcgcggatttttagtccagactggctgggccatccgcttcaataagttagaggactacatacaacgatggacccaattggcaatagtcgtggtaaacttcgaaggggcggtgtaagattcaagctgtagtcgtgatgaaggagatcatcgtataaacag

SEQ ID NO: 257 - DNA, 2000 bp, Artificial Sequence
  Exemplary stuffer sequence
atacatctagactactaagagggattatcccagcgcagtcccacccaaacatcaatctgtccctttgttctaatatatctctggtcgcgaatgagtaaacggggctaaaggtccattatttttatgtaggagcatgttgcttattatggcatagcagtcgccatccccctgtcactcgatctagatacatctcacattgattggaaacttctacaaaacgttagtacttaagatgagtgatttagtgcatttctcgttttcacaaactttgctaaacaaacgtattgagtggcgcgttttttgatttgtcgcataaccgtttactccctgttcgaaggaaatcgatctccttataaataatgagtacattatacagctagcataatctgcgtgtggcaaaagtgaacgtttaatctacaattgatggaaaaatagcccgttagtccttttaaagacgtcttggaaaaatattgagacaaccttcgtccaaaatatgtcaaagcttcgtcacatcttttcacctattactaactccgtagttcaactgactttagagggcaagttttgagacaatatcttagggctgactaataagacggttatatttcaagaaggaaagatcttaagagtcaaaaaaacgtcagggctatcgttacgatattggtatgaacagtaatgatatattttgcagatcttaatataacgacattcgaacacaatagcgtcagacaaaggttaccactcctctataattactgcagcttcaattgatgagcgtcatttaattttggccggacatttacatcgtgagctggcagcacgctcagctttattgttcttgccagaacattacgaatagccgttcaatgccaattagtatgataaaagtagtgagtgtaaaacatggcctgggtttaaagaatgagtaactattattttgtaggaataactgattcccttgagttctatcttaagttgtacagaatcacactcctacagcgaataagcaacgacatagaatccgttatttcgtatgtctcggcgggacatgtataagtagcatacgttatatcggttgtcgcacgaaccgccttcattccaaaggcgcttacaaatctgcagtaaaaagcttagcatttactatagagtatcggcgttgaccgttaagcccgtcccgtccattcaatcactcaattgatcatcttttggcaatagtcgtcatatgagaaaatagctctgtcgttgttattattggctagagtataagctgttaaactacagaatgacgttttgtggaaagtggacgtaagatccttgttcgcgaagactcgcacggtggggaacaattcctgggaatatttgatctacgtacggttattctgcatgtgattacaatatttccaacgcagtccttttgacattatatgaaaccagacccgatgcatatgttttctgactggtggtttgagtcagagtcaacaaaagtatcagtctttcgttactaaatcttcctaagtaaatggtgggcgaccattccttgtaacctgttctgttataggtactattccagcctggaaatcgtggaacacatcgatctagttgtctatctataagagaacactcggttccaaatatgtaatccgcacgtaagagaggagtctcgtacatgatatataacgttgggtacatttcttagacattccggtgatacataatgtacaagtcacatgattacaccagctggtagatagaatacctgagactgggtcctagatgattataacaagtgttacatggacgctctcgttttgttgttggcttaacaccagggcttgctccatgttctcatgtcgttattactgaattatcttccattatgatcctggacggatgaacgaagcagaagataacaaagatgactgaatgccggaaaaggaattaggccctgatatatcgcgcttctttatgcatgtttacgctgtaccaataaacgcaagagg

SEQ ID NO: 258 - DNA, 2000 bp, Artificial Sequence
  Exemplary stuffer sequence
gtacccgtatatcgtcacttcatttgaagctattattaatgtaaaatccttccgtcacacactcttttcaaaaagggaagtctaaattaacattcagatgaaaagcgctgacccacatgggaatatcctttctacgctatcagccgaaaagctccagcgattagctaaatatctaagcctccagaacagagttattatatattggttcgaatatgctaatattacagtagaaagtaaggtaccggcacttttaacgccgaagtcgaccggtgtagctgtgaaaatatatttagtacacgtaatattaattggaaattgatgagatcgaatcttcaggagaatctgacgagcattactaatcgcgcgtgacgggaacgttaatatacaagcgtctattctaggttataataaactcctatctggcaagttgaatggttttttcaaaactttaacgttctggctatacaaagctagttgctttaacttatcgcatactatgatccttcccatcaatcaatctcagtgactataaacgcaagtgacacaattgtctgcgttccacatttctaaatctcttatcgctcattccctctacacaaagttcgattaccaaacgcgggtctacacacaagcttacaaggattacaatatccaattttttgttatcaaaggcgaactcaacgaatttaatcgttggtcattggtatggaatggcgattataagaaaactcttttagtcatagtagctcgagatgaagtgaaccgggccagtcggtagtttcactatcgcgcagtagtcacgatcagttcttagaatctatctcctaatcaagtccaacaagcaatccgaaatgttgctttctataaagggtatgtgtacctgccaatattaaacttgattcactcaatagtgattttaaatatgtccatatttatgcaagaatcattgacattagtaaattcagccgtgcatttgacacaataaaggtagatttagactgcatatttcccgcatatttattattgtcaacgcacaaagttgatggaccgaccacgatcgcatcgaagaccgtctaaacgacgatattcttcggagatccatatttgttttcaattaccgaccattgttcatcaagtgtagttcagtcggaaatttttcgtgtgctttttaaaataccaaatctgaggaaaaagctcgctagatgttgagtcaatccgtaagaatatgccccaggagacatatgtaagtcacagccgtagactctcggttaccccacgatatgttccatatgcaacgtttgttgagtaatatgcagttcagtcgggcgtattatcaacagacagactggcacagtaaattttatcatcgggtttaaaatatctagatacctcagtttcaagggggagttgaactttaacacgagatcaaactacatacacaagattatcagtgggtacgctgagacttatccttagcctggagagagtccagctacaggaactgctagtacttagcgtgcgacctcaaatcgagagaactaattaccctgatcgacagatcgggcaagttaagcaaacgcggctcgcgtgtagaaccataacaattggagatgctcctgcttaagagattatagaaccgcaacccatcaatcgtcagttacccgagggctcacgcacgcggtgatggaagttagttcctttgtacgcacgagctgcaatacgtggtgattataatcggcgcacactaaaggggtggatacaatagtagaagcatatacgtcgcataggcgtacgcgggcgaaaattttaatcgttaacgtggcactaacagcgttttgtctccccactcgtgggttgcggtgcatcgcacatattcccacaacacctcttaatgctttattatttgtattaatggcgcgaatctgcctgatattagtattcgcactagtgggtaacgaaatcttagtcgctggctactgcagaactaattgcgttgcgat

SEQ ID NO: 259 - DNA, 2000 bp, Artificial Sequence
  Exemplary stuffer sequence
actagctacagatctgtaatagaaaaatgcagatgcttgttctgcgtcgactcgctcatcaacatcctgtctcacaagttatgcatcctgtgcattttattgaagctttgatggggattagatcgtgtatggaaatgtttattcgcctggataagatctgtcggcttattcgtggccaataataggtcaatttgcggaaacataaagactcgcataccaatactcgcttatcctgaggttaaatttagtgtatgtagacgaacaacagtatttagtagtatgacgttcccccgtattgccagaactcctgaatatttggatatgaggtatgactacgaaaaaaatactacgttgctcataaccattggtgcagggataccgaactcattgttaagggacgccacagtccagtctcttttcgttcagagcgtgtttttcaaagtgcttgtattagtgtggacagagtttactgatctctccgcacttggactgattgtgatcccgatcatctcttttcataattgtaacacgctttcatagtacacttctgtacattgaagagtgcttgcagccggacagtcctatagaatttggcgtttgttcggccaatgtgtgcattttaactttaggcgccatctcttgagattactcctttgaaaaattttggcggaggttaactctggtctttaacataggcgtgcttaacacgagctttacggtcaggtacaggtaacaaaacaggtctaaatttatttaagcagcttctgatactttccaagggtcacagttggggagccttccgaggtatgacaatcagttttcaaaaggtgtagaatatcatatattctatctaggccagagcattctaagctgttaaaagagtgctatgctcagaagttgactgttctaatcgaaaatcggacatagataacccgcataccacaagtcccgttgtaacgtacccatcgtttttgattctatgtctttgctaatgattggcgattgagacatcctacttctgtagcttggctgttatgcgatccaaaatggtatccagtggtggatgtccgccgcaaactgaaactccctatcagttctttgaaattaatttgcgggctatccgactcattctttaggaattaacagaagaacacgcgtctgtaccaaggttcttctttgttatatcacataacaatgaatcacgttctatgatgaatccaggtatagaagttgtaggtaagcacttgtataagggggcgctcctctcagattgattcattatttactaaaaaaggagcgtgttattacttctaacaactcctcgccattatatattatttaactaccattcccactagaaatggatatcgtgttctaagaccctaattgtgctcattaaactaactaccgcaccaaccgccttgaatcaccggaccacactagttaagctgccgatacccaatatggtattttagtgtataccggatatgaccttatttacgaatggattgagctcaccccatagatcagtaccagcgttattatgaaaatcttgttattttaacagagagacatgcttggtcattactacgaatttgagtttacgttatacaaggcgatccaaacggacaatagcgcgatacgagattatagtaccaatagcacgaatcagttttagcgatctcgtccgatctgtcaagccgaatgactctgaaacgttagtatctgaaacgtttcattcagcctaagatatgtatagtatcattataccgtgtgggtagaacaatcaaatgcagataaagctatttaatgcacttcacataacctctccgttggaaatccatgtattctctaatcaattgaattgtaccttagaaagcacagggggacacctgaagacctcccatctcttaaggttaccggcacgtgaaacttcaaaagtcagacaatcaaacggcaacgtgaatgtcttcggaagtggtggtatgcacatcgcgtca

SEQ ID NO: 260 - DNA, 2000 bp, Artificial Sequence
  Exemplary stuffer sequence
ttaatagaagtaataagtgctattggactaaaatcgcgtcaattagctatagaacagctctgtgacgaactatcaatggggcattcgttcactagtggataccgtacaagctcgccgtgatcgtgcgtcaaggatagtgccagagcgccgcgctatatgtgtaacgacgcataagtagatgtttatgttattgggcaaagtcattcttatccataataagcgctgccgataaagattcatcagagatattgagattctccatacttgactaatctctgagtaattaaaatatatttctaatcggataagttagggatcaccgaacccaatgaacttagtttaatgtgttctcgcgaatatccccatgatataaagatccgaatacctcagctccgtgcgtgctcgtgcagtcgtgcgttttctatgaatcaaccatcagtaacgagtagcggtaactacttctcgagtttaaccaaagcctatgtatactagcgtgcaatcacgtgcggaaggtccgacctacagcagcattttcgttcgaaaaacgaaaactaatgtgcactatgttgaatgggcattcaggccttaacttctaacgttaaactagatttgcgattattaggtatgagatcgaccaggtcgccacagataattaaagatagccctagcaaagtgataaggtccggatgttagaacttgcaagagtgtgtaagattatttactctcggtgcgtcgacaggcgaaacccataacttttatcggtcaagattacgaccttcagctagtatcttgagatttgaaagggcctaaaagcaatttagtgtacttgtgtaacataaccttaattattgatggttctatcgactcccagcggtaataatcttgtaatattgtcggatttagttgaagggcaggttgacataccgaacaatagctagtatcaatgtataactagcaggcatctaatttcgtaaacactcctgacacttgtcgtgtctaagcatgttaggacaaaagaccagtttttttaaacctgactgtaccggcaacgccacagattttatgtctcgcatacgtacgaactgaatttgagggggctcaggtttggacttacaccgcacgtgactatactgagatcgaggctccattaacggcaacataagactagcactgtatgatctgaagccaggctctggtgaaattgcgggtagttaacgacatttatcgacgaacccttgataaaaagtgattatgttgtatctgcgtgatatattcttttcgtgttcagtctctagaacttcgtgcgtaataaagattatagaggaacggttaacctcattacaagacggagaccgttcatagacgccgatggattacagggtctactatagctacctagaacactggtgaacatagggataacatacaattaacaatattccgagccaaattatgtcttgagtcttggttgttatctatatcgttattatgttagaaactaataaatgcgataagaactagattttacagtagatccaaataccggaatctatcgggacgattgattaagacttactcaaacctaactttagcccgattttgcaattagagatacgtcgatttcgagacaagagtagcgtccccatggcaaatatccacggacagataatgacacgtgagggatggcaagagtagttgctcaggatgtaggcgttgatggtctggcgctaatgtcgtggctacctgttgagtctcgcgtaatgactagtagtgttcgaacgtatgaccaagttccttcctagtgttaccactttgacacatacccaggggtttgccgcatgtcgctactatagtataggtgctgctatgaagcttctgaatcagcggctaacaagtacctaagaaaattggacatcttttggatgacagtgcacaggagcctatactgaattatcggtgatcgatgcttcatgtaatcaaaaccagcgcgtacacacttt

SEQ ID NO: 261 - DNA, 2000 bp, Artificial Sequence
  Exemplary stuffer sequence
tactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagataaccgcctatgcggccatgccaggattatagataactttttaacattagccgcagaggtgggactagcacgtaatatcagcacataacgtgtcagtcagcatattacggaataatcctatcgttatcagatctcccctgtcatatcacaacatgtttcgatgttccaaaaccgggaacattttggatcggttaaatgattgtacatcatttgttgcagaccttaggaacatccatcatccgccgcccttcatctctcaaagttatcgcttgtaaatgtatcacaactagtatggtgtaaaatatagtacccgatagactcgatttaggctgtgaggttagtaactctaacttgtgctttcgacacagatcctcgtttcatgcaaatttaattttgctggctagatatatcaatcgttcgattattcagagttttggtgaggagccccctcagatgggagcattttcactactttaaagaataacgtatttttcgccctgtcccttagtgacttaaaaagaatgggggctagtgcttagagctggtagggctttttggttctatctgttaagcgaataagctgtcacctaagcaaattaatgctttcattgtaccccggaactttaaatctatgaacaatcgcaacaaattgtccaaaggcaacaatacgacacagttagaggccatcggcgcaggtacactctatccacgcctatcagaatgtcacctggttaatggtcaatttaggtggctggaggcacatgtgaagcaatatggtctagggaaagatatcggtttacttagattttatagttccggatccaacttaaataatataggtattaaagagcagtatcaagagggtttcttcccaaggaatcttgcgattttcatacacagctttaacaaatttcactagacgcaccttcattttgtcgtctcgttgtatatgagtccggggtaagaattttttaccgtatttaacatgatcaacgggtactaaagcaatgtcatttctaaacacagtaggtaaaggacacgtcatcttattttaaagaatgtcagaaatcagggagactagatcgatattacgtgttttttgagtcaaagacggccgtaaaataatcaagcagtctttctacctgtacttgtcgctacctagaatctttaatttatccatgtcaaggaggatgcccatctgaaacaatacctgttgctagatcgtctaacaacggcatcttgtcgtccatgcggggttgttcttgtacgtatcagcgtcggttatatgtaaaaataatgttttactactatgccatctgtcccgtattcttaagcatgactaatattaaaagccgcctatatatcgagaacgactaccattggaatttaaaattgcttccaagctatgatgatgtgacctctcacattgtggtagtataaactatggttagccacgactcgttcggacaagtagtaatatctgttggtaatagtcgggttaccgcgaaatatttgaaattgatattaagaagcaatgatttgtacataagtatacctgtaatgaattcctgcgttagcagcttagtatccattattagag

SEQ ID NO: 262 - DNA, 2000 bp, Artificial Sequence
  Exemplary stuffer sequence
ggccctatagattttaacctaagctctagcttgtgtgtgctcagagtactgctcataaatatgctcgataaaggaggtaaggcatatcgtaatttggaagataataccacacttattggtaacacgttggaatcacatattaattatgagccagccttggcattcgagcagggatatgtgggagtatcagttgagtttggctccttgctactgccctctgatgctctgcttgctctagcttaggtcattaatgataaaaaagagccagagtgtgggctaaacaggcaacggtaccgttgtagagcgaggtattgctatcgggagacgtcgggtcaaagtgggattcatgcagtaagtttgccaaagggtctgcttaaagagaccgattccggaaggctatatgccatagcaaggtatgcactgcattgagctgaaaactcttgagcatagtatttactaaataaagaatctgatatcttctagcgtgttcactggactattatttagatggtcgccaacaacaagcgtgcgaatcatatagacccaacccagggtggtattgaattctatattaaaatgtctcgcccttataactctctaggtttccatagtacaaacctaggtgtcgtcaactgcatgcactgctttttgtatcggtaatgttgatcgacccgatgggctttttttaataaaggtcttgtttagttgatcatactaccaattttggtggtcgatggctcaatgaccaatggaatctttatagtaaaagagcccttggcaccaacgaatcatggaatttaggacgatgtctcatttaccatattttgcattcagactatgactttcaataatagaatatcatcgtcaaacaccgtggatatggcatcgacaagtgttgggatgcccactgaataacgtctcttcgtcatctttagggcggctatccattaaggaggattttatttttatagcagtcttagtccgaggcattggcgccaaacatcggctcaacactagacacgtctttaatggaaagtatctagtgttactgcggtacggaaagcaagttcagtacttttatccaatctaagtatcacccagcttatatttaaaagctaggtaatagggaagttactaataactcatgcgcgtgtagtgtagtcttgctgtcgcttaaagcaactgaatgaatgtacggctgacaaaggcttacccaagaaaactctcttgtacgctacaagaaacctgtaacaagagaaaaatattttagcccacgtatagtgaggccaaacttgatgcccgtaaaagcaaacaagtaatattcagcagaatttgcggtcattcaagtgtttaggtacgtaacttttacagaattagctgttgattaggtaatactaaatcaaaatgtcgtaataccgaagcagaagtatatgatctaatttgtcgcctcgcttcatgctacgaatgttacttcgtttattacagctgcaaacttgcagtgacttgcatttgataggattcttcctagggaaccatactgggccgcggacagggagtcaggaactcataacggatgaagatgtaatctctataggggtgaataacaggattgaagatagtaatctaagtactctcatctcgtggacgactttaagcgcactgacagcgactcgcgattcgacgaacacccgtgatcgatttacacgttcattctgaaagatatacaggtaataattctaaaagataattgagtaccaatatataggttttatgatcttaggcgcatgtcactgacgagagaaaagatagtcttgccgcctctaagtgttctatttctggacgtgcctgggcattaagggcgacgttgacttttatacacatttcatgtccactaacaattttatatcacgtagcaggacataaagggaggactctataaaaagtttcgctatatacgtacagtacgttcaaaatctccagaggaaagcttgtaaaaaaag

SEQ ID NO: 263 - DNA, 2000 bp, Artificial Sequence
  Exemplary stuffer sequence
cgctcgacacgagtataacaaatatcgatagatgctatagtgataaggtataagtaaaatagtactgcgaatacaaatagcttggagaaatacgttcatcctttaacttcaaaaatttttggacctcaggcacgttgtcattattactggcaggtgataccacccaaaaatcgtacccgcaatatatcttcggtaattcttgccaagttgggattttacatacttagtattaatagtgggatcagcttcgatcgaagaccataactcagtatgtgtattcctcatacaagatttctgaaggacgaaggctcatcaatgctgaggtgttatcaggtcaataacaagccgcattaacgccgtaaccctaatgccataattctttgacgaaatgccaaatagtttcatcaggaatcacattatttggataaggaagcacaacaaacgctttaatctatacccctagaattaagaggacagcatgataggctttgcaatgaaccagtctcctaagcgtaccaccactccggagccttatggcgcgccggtattatggcgatgcactgcctgggcgaaactcgagtgaatcatttttcccgatatacacagcagtacgccgacggtctggtaaaaaaaacgttataggctttgaccgcatggtgatcgtggttaagtgcctttacctagagtgctgctagatgtaacacaattgatctgacagtttacgaccttgtaatccaagaaccatatagatgacccgctgagttagtaagataatgcacgctccggggctaaatctagtgcggttcatgaataccgaatcaactacggttattggctgcggtagaatatttagttgtgttaaatatactctaagatgaacatgtatcactataatcactcaccccctctgcgttcataagtaagtggctagtgtgatagtaacttgtatcagcgaccactactatatgtggaagcttttgaatgagaatctccgcacatgatgatgtattgatacaattcttttgttcgaaaaagcttcggtgttttttaggacaggagattaacgctttagagtcatacatatatgtcaagaaaccggggaaaaaatgccagcccagagtgttctaaacgataggttgttcagtttttaataacccgcgacgcgtcaagtaacgtcacgggtcagctacgattaccaatttgctataaactttcccccgacgagccaaatccctcaaagctgccagataaaaggatagcaacctgtactccccgtcaaatctaatgcattcttgttttttaagtctcgtgtaacatgcgttggctaatcttctctaccgggtccagtgccctttcagcttatgcctcacctttgattagtaatggacatcagcttttagtcacatcggagtgccaattataccgttatatctttctctgatgcagaccgacctgtcgtgtaccgattcatcctagggtaactagccgtggcaaaatatctttatcgtgttgtcaggacttggttgttatatactctagcccgtagatttaaaataaattaagtgtagatcgtccaaatatctaaagcaatcgcagtttttatcacatcatgtgttaaaatgcgatcaaaagaaaaatactgttatttcgagagtcaaggctgtgaggaaatatgatgaagactgccatcctggtggactggcggccccaacgttgaagtttctatttgatcggttattaaaggatactcgagaacaacatcgaaggaataaacttttatagaaagtctccgaaatgaataacttaagatataaatttatcgcgcgatagttctggtggatgatagctttattcctcttaatgcagtatagctattgcacctattaatttgtataataacgtatcatgttagacggtcagcatgatattccggatagtggaagcaaattacgacatctaaatatgtcgctagtatttgagtcattatagcttcgaggctt

SEQ ID NO: 264 - DNA, 2000 bp, Artificial Sequence
  Exemplary stuffer sequence
ctctaacgtgcatttcttcgtcgcctttgtaagaccccacaaaaacatgacgctttagggatatggtccaagactccgaattgaaagtatgctggtatgatatgggacgtttttgaaacccccctctcacgcgggtaattgggtttttagttagtgtatcatagtaggtatatctacgaactacgtctgactgagagagactttgtgcctctcaaccgctatggtgtcagcgactgatattggagttatttacccgtcgttatacgtgggtaatctttactacggttcaaggtaactaatctagtgtaggtagaatgctgaagaattacccgttggacccggtagtccgtccgctccacgcatggaatgcatgagtaacgtctaggtgaatatccggagtgcataactttttggtatctagtccgctactggatgcagaatgacatatttttttcgagtgcttactattactcttctcaaacagaacgatcattatgttgcttaaattcacgctatgttctcgatgtaaaacaattttcgtagagaaagatgcgtaaaacgcagagttagcatataaaaagtacaatcaagcccgaagcactcacaagaaacataggggctaaatgttaccgtccaagtgagtaggatttaatatcaagccgggcttattgggtacagtacgtggacggactacgacgcatgtgtgttatagaatgaagtgcctacaactgaagcacaattactaaaggaatgtacctgggtttacactaagcatcccatcctcttcgcggttcagcctgatgtaaacgtaaatctcgtcttcccattattaagacgcctcgatctacgataggtgatacgtgtacatcggtggaccatgtgttttgatattcaacgatgtaagtatggttccctgcagtgaacccctcttcaagtcgtcgatgtacctgcaagtgtacaatcggaagaccatgggtccatatgtaaaaataagttaggggtcttttggtctgtgttggttataatcgatattgccaaaatattatggacagttagttcgaattttgtgtatggtagccgtcgaaaagggtggacgttaagtatatccatcccagcggctgggagatatgtagaccgacgagtgttaagttattccacttactttaggacgaaatcaatacgattattttacatcggaggacatgacaacaaaaaactactcggtttcgacaggtggaagatgtcgctgcgcaccagtagagcttaggagagcgacggtactcatttgcagcatgggtacgtaatcacgttagtaaataagtaagtatgccttctcttatgtcattttataagctataatggtgttgtgccaacttaaagattgacacatgatatgctaccagataagcctcgagtcgcctatattttgctactaaacctgattaactagagaataggtataatccctggtaaccagtaattttaatactatgttgccacttgatgtagacctggctgtggttactaaggtgctttgaaaccattgaccacccgtttctgctcgggttgtgcatctaacgtaaatattcagagataacgtggctctgctattatttttatattgcctgctgacatatcatcatccttgaatggccagcaacagttcttgatcggcagaggccccatgaactagggtaatatagcagattaactatcggttaactgtattaaacttgtgtaatacttatattgactaattgggattgcctttgtcgttatctcgtttatcttgaaaacggtgatgtttttagaggcgatagtattgaatagctcgaatgatcaccagccatcaagaatgtagctaactccgaaactccttgacgagagctcaagcgaatactaggtcggcgctgctatccgcagagttcagggttctacccggggtataaaatcccattgatcattcagatattatggacttggcgtttatgcgacgagtc

SEQ ID NO: 265 - DNA, 2000 bp, Artificial Sequence
  Exemplary stuffer sequence
aagaagcagctagtgctacttcggaatagttgtcgtttaagtccgttcaaacatgacgctctagtcattttgaaacctaaaccagtaataatagactgactcagaatgattatactgctatctctagtttaaggagatccagcgaaataacttggtgaactatgccgagatactataaaaagatcaaggacgggtcgctcacggttttggtttattttactacttcttcgtggctgtattagtcgatgcaagttctaataaatagcaaacgttttaagtgggattagtacatattgatggacgtccaccacgtcaaatctcgcagcgtcatagaaggagctataaccattcactgcgactacgacatgtgtttgggtagtgccaactacccgcttccgcgtccctgccgttctgtacacttataaaattgatattttaatcagtggatgtgctgatacggggcactgagatgatgaatagtattaggctgtagtaccttatgtacgcaagaaattttagagtaaagattagtctgtgggtaaggaaaaagctaagttatgattatccatggccatggcatctacaagctgatgaacgtaccaacattatctaatttaagaacttaacttgtcttatcctctcttaaagtcttaatttgcactattaagcttagggaagtcgcaaccaaactcgtgtagtattgagataaattattaaactttcttagtatctactgatatccgtatcaagtatgcttataaattcttgttctgcctgacaggctagtgaatcctgcacccgggacgattgcaggtgtatacaggccctcacgctagcaatcaataccaatacgaaataagggctaacatttttcgtaacagattagaagcagtcccgttcagaacttaccactgcaccaacggaggtactgaattcggactcatagaatcctcgagtagtaagaccgtagaagagacagtgcatattaatgtcatagatcaatttatattttatatggttgcccatttcatgatacccctttaaatttataacttagaaaaggagccgcactaataatgagcggcatgctgtaaaaaagtaggccaaaacgcaagataaggtacctttgttgtccaatcaaattaattgatttattcttcgatcgatcgaccgtcatagttgaagtaactatttagttacggcagatacagcgtatcaattcattcggtgactttgcttagataactgctcgataatccggaattatcatcgttcaaagtccttcccttactaaggctcttggattcagatgatcggtcatccctaacaaacagcccactgccatgctgctatggtgacattcgttactacattgatttctgcagaccttcatccataatacgatggtaacgtctcgcttactatgcacggtgtgcccctgcctatatcttcacgatataccaagtggagaaccgtaggcatgtagtcattcaggtggccactctccttcacattatgtttagaggtcatgaataaccctaatcgtgtgacctcaaacagcatcgtattccgaataagtaacaagtaggggtgtttcaagttgcatgacacaataggatatgattctcaaccaaacttggcaataaacgcataggtttagcagtactaacaagccattatgtttaatatagagcatggcttactctgtcatgttcaaggtggctaaacccaacgcgttaatacactcatcggttacagtgtttttagaagagcaattgatatctcttcaggtgatacctggttcattatcctaattcagttggttcaggaagccttataactaccaattcgatatttttaagcatatagattaggtgataccacaccgtaggaaattgtgcagaatttggtgtctagaaatttaacattaagtgatcagaaaattctctgtgttaaacgactgttgcgaatctgtgtctttcaacctcaagtacgatctc

SEQ ID NO: 266 - DNA, 2000 bp, Artificial Sequence
  Exemplary stuffer sequence
taacaacctgtaactgtcaactaatgacctccttaccaaaattgagggtagttggttcaaagagaatgcagcatgacgcagagcttgtagtcacatcgttcttctagtacgcagagtgtagagttaagattattaaactcagagcacgttgtggacaaaccaataccagtccattcaattacatggtatctaacagtatcgtacaactttaatatggtctagggctagtgaagtgtaccaactacttgatacgcagtaaataatttcatcctatctttacgtcgccatcgaaaagcaaagttatggcgcgtggaaattcagatgaaccataaccaaacagataaattggcagcagttttttgtagacatttatataagaagagctcgaggcgtaggttaattctatacaacgctatgatagtcaagttctacttgaccaactacgctgggaatgtttattaaattcaactgggggcaaactagcatatactgtctgagtgtccttcgatggttctatacaaacggggtgtcgaggtactagtggaatggagaaactaccgacaaacgcatatcttatcttctactcgggatttatgaaattttttgcgtatactattcctgtgagcaatgttcaacagcgtagtgagcctcataacgtcacatcaattgtttcacgtctgtggctatcgagtattccttaacttaactagagtatagacattagagtctaattctatgcaagttagataactactactactgtcgtacttcattcagttcctgctcgtactcggcgacgctataaccggcctagtttgtgcgtcgccagataactgttccttttaaacgtataaaaagtacgaaagattaacccagcggaagttgggccccataaatgtcatatagggactcagactactgttaaaaactcctagtatacattgtagataatcaactaaagttggactatcaagaatcaaactgtaatcaggtcacagaacaaatggactaatagagctatctaatcatcatacagatttatacccagtggaaacaaaactttaccccttgaggatttactggagttgtgtcaagttagaaatcggtcaacataaattagaaaatgccttggaacgctgtataactgatcacatatagctgtgcctaatgcttcaatcgtcaatgctgaccacaatctacctgacttggaaatccgctacacccatatccatatacttaaagaatccgtactttatatcctattcaccgatgtccgatgtggcgctatgtgtgtctagtagtatatcagttcaaggcgagaatgaagaagaatacagggtctctttagagcactgtgtcactgtttcttaggccagttaattctagaaatcaaataaatgaataactcgcgacggctcaaaagaaatctatggtttacgcataagctgtaggtacttctaagcttgatttgcttccgggggatcctaatctaaatgtgaaggggcagatttagatctctgctcattgagtgggaggttggacattgaacatagaactaccttccctgcgtgctgtaagattatgagaatctatgctcggtcgttgtctaaaaatcagactacaagggtaagaataataacagaccgaaatagatgtctccttcaagatagtcagtttgcgcaagtctggcaggaacgttaagtaatcctgagttataatagcgcccttttaagctttcctggcgaaaaccgaaccaagcccccgtaacacaatgtcactatccgtacgaaagttagtgtaataacgactgtacctattataagcacatttggttggctatcttctccctagattcctggcggaaaagaagcatgtctacgttcgataggactcatttttgaggaaaactattataacggctataacgcgcgattaatccctgtcggtccatcattcacgtgagtgtaaaattgtgattagtacttaaacgggttcgtgga

SEQ ID NO: 267 - DNA, 2000 bp, Artificial Sequence
  Exemplary stuffer sequence
ttgacgatttatatagctactacttagccttactacatattccggcgtgccggtagatatgactaagttaatacttacagacattcaatattaggatttcggtgacctcgatctctcttgattgaataaaaaatggatattaatgcgtcgatagttgtgataagttatgtatgatgtcctgagggacatatgataatcttctaatagttaccttaaaccgaattgtgtttatgatgaaaaatataggtgaagttagcacctatcaccagactttgggatagttagtccgtaccaagcagcagttcaactgacaggaacgtcaattctgtctctcattactttggccatggattgaaaatcgacttcagtctgactcacaacagttatagaaggattttggctcaccactcttcgaaataggtcatttaatgcgtactgctttttttgacggccctttattcattctattgagggaatccctaactttagccacacgcaaactggtttatatggatactctcaagattgtttacatatccagaagcttatacttcctcaatgtgatgcacacaaggtgggatcatcttgtttctacaatgcagaatgaattaaaaatcgcccttcctggcacatcttgctgtacggctacagagtaaaattagctcgttatttatgagtgtttacacaacccaaatctaagtcgaatgtactttaaacttggcgtggattcatagacatgcaatcagtgttaaattgtcactcaaacacgtgcctgacttcagacaaattcatggattcaagctgctaatattcacaatagacgagataggggcgtagctttttctgtacgatgggggaatatacgagcatttctatgaaccaaaacaggcaaaatgagcaaataccttgtgcatcatatagtttccatcaactggagaaagcctcttgatcggctacaacttttcaagtccttgcggcgttggccctgaagtactatagccttttgttctcactaatctagccaatcacttgttgactattcttgcctcacccatagagtggtaatggaattccaaaaacctattcccgagtttaacccgtattgtttgagaggagttcctagtgtcttcattaaattgcacatggactctacggaaattactttttattaaatcatagaatctctgtcatcagtccatgcgtcctcagtcaataacggtcgccgtgtctacggaaaggttcattctatgcctgtaaagtacatctaacacaatttagtgtgggtcttctactacagttcacccgggaaacgttttatgtacgagtgttggtaaagcgtcctcatcaagtcgatccattgtaaggaatcgactatatactccagcttaactaggaccccgttacatcttaatggtaggtctaagaggtgataagactggaacctacatcatgagttgagtgagcaatgagagccagcaaatggtgggaagactagaccaacacaggatctcatgcttcctgtagcagtgcaactcagttcgctgcgaaaataattaacatatcccctattggcaaaaccctgcatacgtatttagcaaatatctgtaggggtcgtccaatagcagtgccgttttataaattgggttgatacataacactgaatcaagtgaaatcgaacggtggtaaaatggcttgaaaggggaagttgtttaacattcgctagcgacacatgttgcatggttagggttgctatttcgcctcattctcgttacgacattctcaaccagtagcccaccaacccaattaaggtcacgcacgaacctatcatccacttacctcttacaacataaaatagtcaatacaccttcctcaattagccttaatcaaataaagctagttatttttgtctcctggggatcagggcgcttacttcgtactcgcttcccccgctaggaaggccactggttcccgaagaaacgtgaataattgcacatgcttta

SEQ ID NO: 268 - DNA, 2000 bp, Artificial Sequence
  Exemplary stuffer sequence
agtatggaaggtgcctcggtaattacggaaagagcttatctgccggaaacttttattttgtttcatcaaaaggttatacgataataccgcatctaccttttcgtatcaaaattggtccacaaatccaacttattgtcatcttgaatcacacattcatctttccgtctaatgaaggagcgtcattacttgttgtatgaaacgcaaattctctacactagtaagtgagacattaactacagcctattaaataattcaggtagactgatgagtaatatttcttctatatatatgtgatactcactctctactgagttgactagtggactctttgttcttgtacacacacaacagagaaatgcctagaacaaagtcaaagaaagcgcctagatgactttgtaaattgcaccagatctgaagtcgagtcgtgaatagaactttgcataagactctaggacttccgatggcgtattatacttaggaaaccaagccggtagtaagaatcgaggataatactctgggaagtcttccgtatttgcgtcaacaaccagcttctggatcaagcatttcttaactagattaagcttcctctttcgttttaaagcgttttacttcagcaattgtaatccctacatttgtattagccgaatagaacgatgctcctacaacaccaggccgacctcatgttacgatggccgagaccataactcttcgatgaatcattagtggaagagttatctactgacggcatgatcctgggacatgaaattggaaagcatttgcacacgttaattcgccttttacttcaacgctcggacccggtataagataaaattagaccgttatcttcgtagatcgtaatacgtatcatctcgtatatgccgcttgtattcaacggtttcctttttagactggagcgatctacgctggcttggtttaaggactatgctagggtttgtacgtaatccctttaataattaacgaccgagctgacaaactgaataagtacagcatcaacaggacggttcgattgacagctggaaacctattaggcatcttggcccttagcataagtcccagtattatttgttcctccagtaaaaatctccccggaattagagcagcggtgaaatttatggacttgacctttttggtttagtcgtagagggacaaatatcatctcatctgaacgctcatcaccagttagttcatccaaattcaattaggaggcgtcatattgtcgggcgtctgtaacggagccagatctagaagttcattgctataaagaattagtgtgcttggcacatcacctaatcaaattttgggaagcagcatagctattcaggtgttggtcaaccagataaagtctatgaagaaaaaaacctgtgttagttctgcgtattagtattgtagtataatgtacgacatcccgaaagttaaattcaggtcgcagagtccctagtccaccgttctaactcacaaatcgatgttcggacatagctatttaacagtccatatttaccttaagtgtttcgacttatgtatgctagttaggtgtgtggctcgccttcccactgttagaccacatctagacggacatcgttaataatatctgatatacacaaaaacgtttaccatagaaaacactatattcatggacactttatcatattcctcgcccatcctcacgacccagataatagggagttgtagtttttctaaacggttttaatatgcaggtccataaagcatgcagtacattactgtttaaaactttaattcagatatatcctggagaagaaaatctcgattggttaatcacttcattgttaaattcgatttcgctatacgtttctgtactaggaaatttttcatattaggcacgcggtgttggttccgtaacactattaatttcctcccggttcgatcatggcttgcggtaagtcctcaatttaacataattgagataccgaaatcaacccagcgtcgcagtattttgagtt

SEQ ID NO: 269 - DNA, 2000 bp, Artificial Sequence
  Exemplary stuffer sequence
ggttaaatgcatgctcacgcctcgccaggttgttaaaccatgtacttactcaatttgacaactgatgtccactctccacctcgcgcgatgctactttcttaatactaacgccaccttgtcaaacacctagatcgttctaagtgtagcaccagacagagtagacaccgtaaaaggtgaaaaggggattaatttctcctccttttgcacaaaaaagttaaggggtaggccggaggaaggttaacgcgaagcacctgcgtaatcggtttcgtgctatatcggagatataccgtaatgactcgtcgacgaaagtcgaaggctttaagctccatgccccatgttggtgcgttaggactttggtaaagtggtaaaatttagatctctttgtgtcctttatatcaagttagtgtgaatgctgagttttctcattttttaatgtaagtgattaatatgaagatgtgtagtctaatttggagaaccaacttaaaacggaataggatcggtgtatcaatgcatatgaaaccggtaaatttagtcctgttgacctgaaactgatgggaacaaaccctcaaacgctatcgcaacaccgtcctaggttccatgcactattaacctgttattgcccgtgcggagatctggtttttattgttttatactctagatatattagcggttatgtttttctgttaatttaagatgcatagtctactttgacctccggcaacgtgatttgtagaaaatatttcccacacacactatatgtgctactcaggttacccatagtttatgtaataagtatcactttaaaccctccacccgcccatacaatagaagcccttcaattatacgaggaggtattgacctgactagtttaccaaagccaaagatacctggacaagttggacaaatactaaaggactactgtagcatagtgtttgcgggccagtatacgcttatttaaacgatactactgataagaaacactggggtcaacgtgctttcatcacctgtccattactccaacagtcccaattttttaaagaaggaattttcgggacagtgaacgcggaatcgctaataatattcagatagatagctcgacacaatataactaatcagacaaaaactattcaaaactttctcctaggtagtgcgcggctcttttacgtggggtttattcacctgcgaattatcctgatgcccaggagcaaactcattataataccaccaggtgacagcctacaagtttcatggcatggctgcaacctgcacacgaacgcttatgcagcatgtgctcttgagttataccagctacttgattcgatatatggtttttgtgaagaatttgataccattgacacgggatgttgcaaatatttaataagtccatgcatactaataccaacgccagagatagattgtcagtagaactcttgaagtcaatatggaccgagtgacttgggtggtttatcccactgttagaaagttatcgtaaaataaattcttggtcaaatctaatccttataaacactctgttattactctgcttcgaatatgttgttattgaccatgctgataactacatcctttatgttaattcaaggcattctctgaaagtcaacaattaacttcatatcagacatttgacctattcctcacttttctataacatgacaatcacggtgattaaaaacatgacgcgtatcggcagcaaaccactgtactgatatgtaagagcgcccgtcgcatagatattttagactctgtccaaatcactctacgccaacttgaggtcagaatgcataccgtggtaagctgaatagttcttatacactttctaatttacccagatgacgattttttgttatatgaatgacgatcttggcattatactgccaagactgcaatcaaatcctaaattcataatttagtaagtcaatagcagatctgaatcccataaatgaattctatcgaagtacctacactatgtcacgtagaacaag

SEQ ID NO: 270 - DNA, 2000 bp, Artificial Sequence
  Exemplary stuffer sequence
ctaggtaaattcttaggtagccgagttgaactattaataagtctcgtctgtgagtatgtcttccgttaggtattttcatatagcttcatgtgcctgtaaagacagaagtataattggatacatcagactttttatcccttttacagtctagaaagacctacttgaaacatgtttcttaatgggtaacgtagtgaattatgctcgtttttcctttggtagaatgatatttatctccatatgctctgagttggataatttgtaaagaattatacacgttaattcaacctctttatcaatgaactacgcgggcttgatcagagtaaactcacaatagtatcttgatcttcacaatctgatggatattgatgcgagttatacgacctgtggcatatcaacaatgaagtgaagtgtctgtccttatgattcgaaacaaaataagtgtccttgctagctacacccacaccgcggtgtgcatcccataaaggctcaggtatagtcttgtcataagcgctacactgccattcgtttagaatcattgtttagcaatctcaaaagtaataacatccgactttcgaataggttcagtttcctgatctactggagcctatatatatgcacagacgaatctcgtacatggcataagcaagtcatgagaagaggctgtaccacgtaaatataagcctctgattacgctgaagcttaataatcatcacccatctacgaatccgattgagggcataggctttcatgtctttttcgctgtaggtctatgcgattgtgagactattgagttttccacaatatggtggtaggtactgagtagggtacatttcactgtcctattgcgctgtcgtatgtctatccgccgttgccgtcgtcgatgttataccatttgactaacagtgttatgagtcactcccttggatgcgatgtaccttctgttgtgagggatgtaagttgcagttaagcactattagcgaataacgctaggattctggaagaagaaaacacagggtcgcttcaggtctcgagaatcttacggttagaaaatttggatctgaataaagagatgtctagccagtgtgggggttgaataagctaaatgtctgcaatgtgtatgcttctgcacagatattaacaaatccgccatatttaggcacatttggtaatggctgacaatcggatctcaagaattctatactgagttatcggactacaactaaaaagatgctatataaaattgtcataattcatgaaaagccagtaggccgaccatcatcgctctaagttgagttgtttgacgcgaggcaacattacgtgcatggacgatatacacgttactagttgtatggtatttcggctaagtttcctagctaatttcattaaaagctgcgcattggtgtttttcagcctatatactgacgtagtaaacttacatacttaattatactaggtaatgatatagaaaatggctgtacatcctttctgaaatgcttccatgcaatggtgctacaagtcttagatttacattataatcggaaaaacatcaacagtatgattacctaggaggagctagcatatccagaaagtagaatagcagaagccaccaacagactgggtgagagtgacgttatgacggatggatcataccccatcttaggagggtcaggtcatttctcaatcatatgtttccagatgcgatgcaaagacaaggcccagaaatttcaattgtaggccaatcgtccggtcgtattaatctcaaccaagtaaataaaaagcatgtgggctgggcgcagtcaaagtcgcttttcttggtccttactaatctgaagaatatacagtaaacagaggatagtggggctagttcagagtaataggcaacaaaccctttcatgcattactgtagaatttgatactattgcgtgtatcgcttttaactttataaagagtcgatacagcgcaggctcataatgtttggagtctgtctaataaacatctaa

SEQ ID NO: 271 - DNA, 2000 bp, Artificial Sequence
  Exemplary stuffer sequence
tttatctatttcatatattgctagataaagttgactgacttattacgattattgtcccagacagccgagctgggccgtgcgtcaatgcacgggctcagcctcctaatgttagcatttgttactcttggaacatttggatatagttgattttttgatagtgcaaaggttctcggtcatccggtataacgatactccctaccctagacattcaatcggtgcgatggtaagtccgtttcgcactgaaagcctgtagagtctatttgatgtttacttaatgcgatttgactcaaatgtaggttaggagtccgttcgcatcctatgcagtgataaactatctagtgtgtttaaaaagacgcaaccactaccaatcagaccagcaaatttacatcaatttatgtcaaaacgcccttacttcgtctaaatatagatatatcaccacatcaagcctgcacttctcacactatgttctatgtcatgtcgttgtaccgaacaattgatatttaaccggagttgaagatcagctaaagagagaagttatataaccaacaaatacagcccacccatcaatgatcgtgaaaacaaactgtacttaacagttcaagaacagtcaccatttctcgacgtacaaaagattcttccattatggttcgatacaaattgttcaaacgcctgtctatagcagggctccgccatatttcgagcatactaaatcattgggtggtcaaacagtctcacaaacaggtctgttgcgattcatacgagacgaccatacttaggcgttgaaatgtcgttgcatttaagtaacaaatactatagaccgctggtagtcgccatataactctggctccagattatacatgacctgtttagaaaggcaatgggaagagggcaaaaccccaagattgttcctaatagttgtagataaatggatgatatctgcatcatcactgtttagagaatcccgctttcctttattcggttatactcaccgtttctcggcgggttgagacatgcataacttctatctatcgttgagaattatcaacttcaattcccgagactgtcattatctatagttgaggaaccttcgtcgctgctattgaatagtaagaacccctctagtccagctgatgcttgtggtaactgcactagtaattcatctgccatccgtgcttaattgggcatgctttgttgcatcccactcccgaacttgaaggttggaactctcgttttgccagcacagttaacagggagtaagacctattggtgtgacataacagttaggtaaatccatctaaacacgtgtgtttactaatattcagtcggtggactaacagacaggagcttacccatccgtggatgttttcttaagggtgtcgttagaatgaatagtacatgtatagtactgtccgaggtgtagatagaataaatgtgaccgtgatctcagatttatggttcaaacgttctaattttccgaggagtagtacatgttggtaccttttcacattatggtgctaattaggcatgtataatatcatatcatagctttgcccatactgactatactaaaattgctattttggaaagtttataaggccgtttctcattgtatctaagacctaagcttcgcgtcaagaaatacccttacaatcggcctatttaaaattattcatttgtctagggcgcgatgatcctttccgaatattttatcgattactacttatggatacccgttagacgcttatcctcctactacaccgtactaattacgtacttttttcgaagtacgatctgattagtgtcgaccaccttgcccttaaatctgatcgctcccaccagtacgcaggacacacgtaacggtttcgatacccagcgagatcagccttaccagtgcttgtgtggtataaccacactatttcaatgcacaatgacaagagtactatgttaattcacatgcctatctagttcaattacgttcagactcataaaatgccattgct

SEQ ID NO: 272 - DNA, 2000 bp, Artificial Sequence
  Exemplary stuffer sequence
tcgaaattggatcgacggagctaatacgcaaattatttgtttgtgatttctatcgcgcttcaaaacctacaaaaaataacagcctttggtgtaattcgtcgtggccataaatatggcttattctatatatccgaggcccaggccataacaaattctccaagatttactaaattagtacggccttcattccgacgggaagtttaaactcaagccatggagtccggtagtctttcaactttgtcgtatgacggtatgctacatgcccccaatccgctattgaacaatggcaaacactacagcagttagccagagaattacgctctttcactttcctagaagtacacaagtcctgaacctaccaactgactgtacacaccctctatggtacttttgctgtttagttgccgaatgatgcatcatgtctgatttttcgggctagccttagctgagtgtcagcttcaccctgataagacaggagtcagaaacggaatttcattaataccgcctaaggcgaaagagaggctgtcatgtaagccggcaggtttcccccttacggggcccacactctcccctcgctatgaaatgacacttcacaaacagtcgctactcaggatttattccaagttccaacgatgttgagtacattgagaatgtattatattaagctaataggcagttttctccaactatcgattattcggctgatatagcccccatcctgagacgttattacgtcactgaggatgatctattcacacaacacttgggttaccatagttcggaatgcgatctaacgtctcacaatggtttttggtggaagtatagtcttattccccgggctatcgcaagcacccaggagtagtttcgttggtgtcatgcttatccctacgacccaccagagtgtccaatcaatttacacctaaactggaacctaatatattaatcaaactttaaatctctatatattcagactactttactcactttgatgttagatgcgtaacaagcatataaacccgtttgtgatcgtactcaatcgcacccttctcgttattgattgatccttgcgcgaggtaacctgggtaatctctaagttatcgatgcaccgtatcaacattcatgatcgaaaaaagtttagtgagaaggagttaatggatcgttccgactaaactaatggaattatgtatgggatgtatttcgtttgagccaattaactaggaactaactcatacatcttgcaatagtggtagcgtaaaatggttgaacgtagttgaaatagtagggatacgacatgtcccctaagcctcacccttggtagttctcgtaagcggacaacgcgttatcatcacgctttggagtgtactagtttatgtctactgcgttcgctgacaataagaacagcaatatcccaattctcagtactgacgtaggaccattagcgctataaaaaaagtagcgtgaactgtcatttattaagcattccattttatccagtgtccgctaggcggctaaattatacaaacagaacggtgttcttatactgttactacctccacaagtgggatttacgaacgcagaaagagataagctcactctcgctatgtgcaccgatgagtcatacagaggtcatcagtaaaggaactcaatctagagttacagtccagcaatccaatccggatgccaacaggcgtaacgattatattcaaccactaagccgcataaagtatcgatgattagcgggggaatacctcctaaacagtttgaccggaacgtctacaatactttgccggttatcaatgaaatatgcggggacgaaccatgcatcgttactcagcctttggtgtacgccagtaggagtactacttgttcttcttacacgacacgtagctacttctatgtatagtaatgtagttgactatagaatgacgaatagagaagggaaccagagctcacttattccgtcaactcgatttatcatgttgttaaaaaagataaaatgt

SEQ ID NO: 273 - DNA, 2000 bp, Artificial Sequence
  Exemplary stuffer sequence
gctactattttagatatgcatcaaagaaaaacaaggacatctcctgtatacgtataggtaataagaagaggatccaacggaaaaagccaccggtggagataataactattgttagcaagtccagttttctgtcaggggcaacgttaagatagaggccagggtaattatttaactactagctgcacttcgacttcattttctgagctctgtaaataccaatggagcgagtagctacggttaaacagatatcggctggatgtcggtggtaggaaaatgtgcctgttgcggctgataagcattaacttacctaaacatagattgttggttttcctaaggttttataagaacgtatataaagatttcttaaatgacaagcttagcctgcataggctacatgtgagtgtggatggcttcgacagtgatcccgcagtggaccagattccattacctgaatgaaaacgttcaattaaaccacttaccgtatcactctgtccttgtagccctgtaaaatgagacttgcggataccaaattagccaaattattcatctaactataatacttcttccatgaaacattaatacggccaccgggaagccaccgattctgtcgccttatattttttgctctatgtctttcttttagtccgacaactaatgtgaacaaatttcgacctaacaaaatagagacaaataaccctatattaatacaacgctacgaagatcttcaataggattggtccgattatagaccaattatacttttacataatatgtacaaaacatctcggcattcgatggcattggcgtggatattcgattgtaaaagcaatggatttttcttgcgctgaaaatgatgatcgccctcgatcatctgtatagcacgggtcgaagtttcagaaatgatagttgctcaatttggttcacttcgaatttacgctgatgtcccaagcgacatgtccccgatcaacatggttgttggatatcaaaaagctgataaaaaatgtgaaaggacacgcctccaacgcgtaactgtttcacctacttccatttcgaggaactgggtcgatttaacgacatcaaagttgtttgctcagacagtcttcctatgaaaatgaaaagtgatctaggagtagaacccgatggctattaataaacacactcttactaaataatttggcgagcatcagagcgtaggtactcggaacctgattgccgttccgctttctatacactgtgaataacaaagtcattgaggtgacaaccttgccgcgtgcacggtctaaagcatgaaattttaaagcaacaatcaaatctctaacggcctatctcaagttacgcagctggcggtaggtgggttttcgcactgactctttaaccaagctgctgctaaaatactcttacctcactgttgatataatggtcgcgattacagataatcccgcacatctgtcaaatagaagatccagtaaagagtccaaatcagagagacccaataaagtaaccaaggcattaccgtttcacgaggtggactttcatgaaagcataagtatggcgtataatataatgttatttggaaaaaagatctccacaacctgttttaccgctgaaaaacctaaataccgtaccagacgaaccacttgatagtcgaatgcgccattgaaggaaacattctccgttaatctgattttaagctcatcaggcttttatctttgcgttatctacatttgacgattaccaaggatcaattacgtgattggactatacttaatatcaatgtacgaaatcgtctacgatactacaaggtaaccactgataattcctcattgctctatgttcacactgaccttgctaatcgacgtggacttgcgtccttgtctagcttataatagtgagatttaatgacaatgctggtataataccgtgcaactacacgcatagaaattactcagcgctcgagaaaagtagattacttcgctccttcggagttttgcgtattttca

SEQ ID NO: 274 - DNA, 2000 bp, Artificial Sequence
  Exemplary stuffer sequence
cctcatttgcccttttatatttacccgagttagttcacgaatgtgccataattctggtcgcagcaaactgcggtgtttagaaataatcttccgttattcgtttatcaagacctcgttgtttagtagttctagctgaatgcggtctattaagttggagaagatctgggttcattacattagaacccaaactaattattaagttctgctcattagcattaggtagaatctattcttgtccggcgctgttgctactgggtttagtctaagtagtactttaactgttcctaagggatgctgcaaaatgagatatactcctccgataatgatcaatttggattttgggcagcggtaaatgttttatagtgtgaattgtgttactaaatttcatgacgtaagctgaccttctaaccgtcgtgcttggaggatttacgcggcgccaaaaagaaatatactagtcccaatcgcactaggatttgtttaaaaaaagacggaaaacctgcaaccaaaggtgtcttgtactgactctatctgcaaaatttggatgttctagctccgtttatggtcgctacatggaaacgctattggttaaagattcactataggccagttcaagtttcccgaaaaatcgtgacggacgttatactctaacattgataagaaccatgtatcaagcgatccgcaatatagggaaacacggcgaagatcaaatttatagatgggaggaagcacacacaatatgagtattagtgtgctgaaatcagcagcgtaaagtgcttctgttccacctatacttttacgagtctcgtaatagcgtattaccatgtaagatgcattaaagctataactttatggcaaaaaaggtaatttattcgctcattactattatttgtcgttttgcataaataaagtgttgttacttcaggaagctttaattctctgtctgccttaacccgaattctacgcgatctccgtatagcagatgagaaccggtgacacgagacccgcactcgcaagtcgtttcttgaggctaacgacaaaatgaagccatcagcgaaatctcatccgttaggctacccaaagttaagactttccctgtatcccgctaatgcgtcaattggtagacgtatcgggattagatattcaagaccaagtcaggtagagttggcgctagttgaacatggacctggccttacaaacaagaagaccacgagagccctagtacaggaatttatcggaaaaaataagaaaattaaaatccccgatctgtgtggtgctcaaataaggcaagggcgcttagcctcacagtcgttactaagtcaaggttctaaaagcacgtgttttagcttgatggatcatgacttcgctacggtcactactccaccgtgtttctggaggtatgcaagggaaaatcgagggatgtgctcaaatctgtggcaaccggagcaccattctaggtaacttccattaacttttgatttagagtatatggttaagctattaaacgtttcctaaggacaagtgggatagtgatatacttttttcggcgacatcaatccaggattatccgctaacagatcgcctagcgctaccgcatatgatgatatccttaggaagagatccaccccggccaagaaactccacactcaataggcggtgacctatttgtgagttatgcagatgtgtttcaagactcaacgccgacaaagttcaccaccagagagtgtaaggcttatcaaatttctgattttatcgacttataaatttgacacgtctaacagattcggcctttgattgtaaacatcgccgctatgatattttcgtgatcctttgggatacgagatgcatcagtactggccccgaatatttccattttaattactgtgtaatgcttaggttcacaatcaacaagtagttcgtgaaaatgttactataatatccacacaaagatttacgcactctaatggtggacgttggacctctgttaacccgctttcgttatt

SEQ ID NO: 275 - DNA, 2000 bp, Artificial Sequence
  Exemplary stuffer sequence
actaaagtcctggagagtatgcttggcctcgtgcggtaacatttgaacagcatgctaggtgctagtagaccctttcttgacagcggaatttgctgttattcaaaccacctgtcaggccaattctggagcgcaacccacagtgatagaagatagtcgttacaatcaaatcccacaacttgagactagccctcagactgcaacagtacacagttatgctgtggagacaaataaaatacgttatgtattggtcattagatttggctttcttatacgtcgtgtagtaatgcttgtgatcggttgccgacatggttacgaatagctgtttattaatttaaaattcaattctgtcgatttagaggatggataatatccgctatgtagacatgagtgagttccttatccttcaattcccttttttctgttattttggatctacgaatgaggtattaagttcgtagcactcgtccgtttcgtggaatgacttattcgagatggcttgataaggaattgtacctcaaaggtttcattgttaagaagatgaattttcacgcccatggcataagcatatgattacgtccactaggtcatagacacatgataactcgtcgctcaaaataatcgaaagaacgtctatcggccaaattattactttgatcccaaaggagaaatcatattggggcgcgggacttcatgtgtattaccatccagcaagcatttgataaaagtaactcctatattattatgaatagcggtaagtttctttgaccaacctgacaataacaccaagtgactcactgagcccgttatctactaggtattcgcgaataccgtaaaagcttgatgcaggtgacaatgagaattatcattagcgtactgtatgctcaacctagcctccttgcaagatttcgttctatctattttgtattcatttctttccgcgacatgcattcttttgctagatcctgggtcctgcaatcatttataagcacgcaacttagcttaaaagtgtggagacgagacgtacaatcactacttcccatcacttcttctcttataagcgtaccgaaagacctcgtattttattaaacaataacgtgcagttggcctaacataattcgatgtctttcagtgttctaggaaaggtgcggtgtgtctagcaagcatgtcagccctacagattcttaacatacctatgtgtctaaatcgagtatactataatgatgtaccataagcccttgccaaaggatcatattcggactagttattgccttctggatggggtacttagactaacattttaaacctcttgcgatacgacctggtgctaatacactattccttcttttctcacgcgaactttcagtatcgtacaaaagtatgggatttaaaccttttgaagtttggtcgtgattatttgtttttagggcctcctcgacgcctcaaatagggatttcttcagcactacatattttgagccgtatgcgaacccttcttaggaccgcggtagtttgttcacgagcacgttggccacaccccaattatccagaaagccggacttaagacatattgagtttgttagtgcataaatagggtcgcatattgatctgcgactcgagtaaatgtcgtactggtgatatattctcccgttttcgaaggccccaatcaattactaattaccctatttacgaatgtcgagagatgttcaaacgaaacatgagggcgcatcccaacgcccattttgaaacttgattgttgtataattcttaatttttgtagattcagcgttcttgacacattttaaagacgtcagttcaccgtacctaccccttcggttacgcgaaaaagattaggttaacgatttctatcgttcgttggttgttatttctgcagtacattaattttataacttgatatatcaaatctgtttttgattaatgtttgaaagcaaatcgtaacaccaaggaatgcaaataatcatacgtggcggaccagctactata

SEQ ID NO: 276 - DNA, 2000 bp, Artificial Sequence
  Exemplary stuffer sequence
tacatccccatcagtcaagacgattcgttaacaaatatcgctgactgggagaatcccagcatgtcttggctggctaaatagaagctactatgttacgcacttccattttgaattacaggcgacaacattaccagacttagttaattattaaacaagatcactttgcgacagtcctctgaggatcagttagagtgcaatcacttaagtaatacaaaaatacagaaggattctctggcgaacaggtttattagcgcatggccaaatttctaatcaacccctttagttagtacccatttctagccaatatcaaatgtactccaagccggcgtatagttgtcagtgtgtgatttaacgaataggatccccccccataacaaatactaataagagtggagcaattatagtttagatcgtaaaggtttaaataaataaacgtcaagcacaattatggactcgtatggggacaaattgagcctactagcagttctagcgaaataagttgacctaaccagtccatggactgccggttcgttgaagtcggtccaacggattgcagatcattgctaggcagttggtagataaatttctagtacttatagtcacgtaattgtcaaaagtcctacgagcgtggtcaccgtattactacgacctccatagttttctaccgtgcattctgaaagaaatatggctggagtgtcctagctcatgatagaaaacgcctacacttagccaatcagacattaatgcggtaacggatcaagcattacagggcggattggtcgcatatcattgcacggaaagcgttgccttaagttcggtacattccactttcaacttcatattgactcaaatagtgggacagtgatttacgcggagttttaatctaaaaattcttgagtttatgatagaacagatctaaattacggtttttatatgtagtggtattaataatgttcataaccctagatatttccgagattagcactcgttcggcgcattgccggtatagaacaatatgtgaagaaatttgcacctaagaagttgatattctcctctacatgcgtataatatatagtaccataagtggatcattattaaaataaatctgagtgggtggacttatcttctgtcaccctaactggatcagcagtgggctagtagccattaaggaacaaccacttggcccgaaactatttgaaaagtgataaatacatacacgatttactacataaccactcctcttgttgataggcatgcccaaggattcgtatgggcgattttccataaacctacagggtgattcgcgcatataaataacaccaaagcagtcaggctttttgtatgaagtgtagcttccctaacagtatgatagttgtgtagagtcgcttctgaactggctgaccctagttataattagttcggcggaggatgggccgcgagacaaagtatactcgaaccttagggccgcattccaaaggttatttagataaaagtacgcaaacccgcacatgagttgaaataatgaagtacaatgttatttattgtgcgtggtaatagtctcgtgactgaaaatttttacctttagggttctctatccggaggagcgtcatgagctcaaatacaaaatcggagcattgactcaattactactttatgacaaattctacgtctaagcgatttttctaaatcgccgtgatcaacaaactagatctacaccagtgatgcatgctcacggcgaatgtcctgaagtcagatctaattcttaagggttggattagctggctatagcaagccatattaatatgattagtcgtgtatggtttacgctacctctccatagatatttctaacttacatttgtaaatgtttccaagcataccgtcagtataaatacccaatgatgtggctctccttcaagtgtttagataatagctatttccataaggtgcctcccctatccgctcatcctcgggtttcatatgttgtaagtggcacttaga

SEQ ID NO: 277 - DNA, 2000 bp, Artificial Sequence
  Exemplary stuffer sequence
ttgtttcttggagggttacttacgattattcaatgtcaagctggtaccaaataatatgttaacatcgacaaccttgctgattctttaactgtacgatttactcaatccttacaacagtctttccccccgatgcttccgataatccggatggaatgtaaaagctttaatttagccataatggagctactctgcaacagtaaggcaaaattttcttaaatggaggccaggcaagatttgtccccgccagaatagcctactccacaatattctctttaaatattcgccatgctatctcacgcatccatgaacaggttatgaaagcgtagagtcaaacgtacactttaggttaggtgccttgtggggatttcacgccacaaagtagagtagaagcagtgtatcaaactatgtgtaaaagtaatttcatatagtaatagccaccaagaatgcgaacataggtgtcggcctgaagatctaaaattatacttattaacaatcatgtgagtaggttggattttaacacgttcataagtatcgatcgcttcgcttaaatagaataaagtacacatcatgtgacgacgcgcttcgattattgtgctgcgttaagagtagtaggataatttttgatagacctgtctataacacggtatttaatccgaagttcactatacaatcataataggatatcgtgttctgtctcgatgatctattcgtcgcttcgggtgcaatataggattcctatatgaaactcacttccctgagcattgggatttcttgatagctagatcgcgttagagtcgggcggtgtatagtctcggatacaagaacataagagtaattatgtggaaccttttcatgtgattgtgctaactgtgtgatattcgcaataattcctacatcttagtttttagactggacttttttttcccaagctctaagcatacattattcgctgcgtatgtcactgacctagaggaataagtgttctgctgtcaaaactaactctctctagcagcctttttgaccatattatcaattacgcgccatcccataataacttcaaaatttgcaaccatcggaattagaaatcccgacgtaatcaagacgaatcttcgccgattatcgagcttacataatcgaaggtgcatttctgaaccttggctacgctaaccctctagtcggggcaagatgacttggttatctggttaactaggaactcctagcctcatattgtatcaatctgatctaatacagcgtctaccaattatttgattaggtttgcttgccctcatagcatcgcagcgagtatctcacaatgtgtatgggtattcttctagttacgagtttagacggagaataagccgcttgtggttaacctctgtaaatacctctagttgaataagtgtgcaacccaattcacattcgtcatgttaacaaatcggcaatctttccactaatgagaaaaaacaaatcattaatatatgtgaaagtaattattgtgtcctcataacggtaaagacttacgagtaggtaacaatctcaacttcaccaattaccacctagattccagcaccgccaacgtaatcagtgttccgtgcgtcttacacaagagaactccttaagcggctagcgtatacttttaagagcagtgggtatgtggcccggggcatctattgtttaccgtaatataagcgcactagtctatttttacactaaatatcattccatatccggttctttcagtaacaaaagtaaacacagtgttttggaagcagtgtatcaagaattgtgaacttctttcaccggcgcagggatccactgtctagagagaatcttaattctatcaaccgaccctccatgtcttatagattgtgtcaacggagcacctaaccgtatccttaaaaatttagaggaaatagaactctcattcttcagcctgttaagccaattaaatcgaaaccgttgctattaggtgtaacggtagatgtgataaaagggtcaca

SEQ ID NO: 278 - DNA, 2000 bp, Artificial Sequence
  Exemplary stuffer sequence
aggacgagctctaggggtgcccctgctgttgttggttatttaaaagccgcgatgaagagaacgctagggggaaaaaacgatttgcctagaatagtggatcggcgttttgatgtaagtgtaattgggtagaaggacttgttttacatttgcgaaatcttgctcggggacgttataatatggccttgaaatggatgatgacaatatagttttaatgttattataattagagtatcgtattattaaaaaggatgtccactgtggatccaagttaagcattaggcgcgttgaagagattgtaccgcccgaaccaatgcaattgacatgcctaactagcaagacaaacgtgttaagactaaagtccctcctatcaacgtacacctcatacgcttgactaggtagaatactaaaatactctcgtaatgaatacctattatctaagtgactgctgcgttcttttaggtggtgaactggctccggaaagtgtgctaatagtctatatgtccgcgcctgccacgtaaccacgaggcggatcagctagaaacataaagccgtttgagcaataagtgactatacttaacggtctgtaaattcgcgcttcaatacctcttactctctgcgttctatcccgtctttttataaattcaactatacgctccattggttatcgccatatgagtccttatctacttaaactggctaccaattccttgctctaagctaatgaaagtccattcgcaggattacaacatcaatgctaactttctcttgcatacagtatatcgtctaataaatgtataggctcccggaggtcggaacagcagtactcccggccacgtatcccgaatacaaaccttattagtaaaggaaacactagtgagagcgtacggggattactcgaaatatcgcaggaaggtggttaatatgccaaggaaatacgaataattctctccgcattccgaaactgttagcacatagacaagacaaagagtttactgacacatcttttgacaacccgcactctacaacgacctactctttatacaagtacggattattgtaacgctccagcctagagagagtaacccggagttatatggagtcgcttgaggagaaatattaaagctgaattctgttacgactagtaacattaccagccgaggtctgaataacgtgcctatggcgatcaggacaatacgagagaatttcttctaccacactatgtgcagcagctcactcaagagtcctatgtagactgtttaaccagtaaggattgttgtgcggaagtgtaatatggtcgagaaataccgctaatatggataagttaattgaacttcggacgtcacattctcctataatgaggatctattcaaatcgttttgaagtaacctcctcatttgagtaaactaggcttgcctggagatggggcccccaactgtaatgtgttatgtttagtttgaactcagttggctcaaagtatcccgcagtactaatattaaatcttgttattgtacagctggcgaagaaagttaagaaatgtgactcctatactattactggatttacaaagtaagcgtctttgacattaattatggtattgacaaatcaaatgagagacagtaagatgatgacattcgctcatattgtatggctcgttgactgatgcaaatagtaccaaaccctttttttagaattccagatgaggaattagatttttcagtcaatagttacttgttatgccacgtaggcttatgtcccctaaatcgcatataataagatagagtgcgaatgcgtgcacgtgtacactaatcagggcaaactaaacatttaacctttggagaaattccgtggcgctgaacttagtgatgatatatgattaagggatccgttttgttttcgataatctaagaactgacgaaggcactaatatcggagttacacaggaaatagaatgtcgcaagatgtgccttaggagtcagaaatcaacgagtgttgatcccaca

SEQ ID NO: 279 - DNA, 2000 bp, Artificial Sequence
  Exemplary stuffer sequence
acaacgactttcgaaggtggctgaagaaaaccacatgataaaatcgcgagtatggtaaaattagctacctgagtatatttaatcgaggttatatcttttgtgagtcggacacaaattctatatttgacggagcatagggcagacggacatataaaattataaacagtctgtacggcggggcctccaattggattcccgcgatcatatcagtcagttgggaaccataaattgcgaaactcagtactatgcttcaatgcccctttctaacacgtttatcgcttcaacctaacggtatttgcactccgactatcgtcttatgcctcacaatcagatgtaataatgcgggatttataaagattttgaaccattggacaactgacggcttctcatctcaccttgacgagagtatttcctattaacctgaatttcgctaaatacttatctttatcgccaataattcctttatgatacacagggcttctccaattcatccacgcagaaactgcccaaatgaggagaataaaaaactttataattaaatgaattttatagcctatgcgtatccccctacttcaaatctgtgcagtgatgataaactattgtaatgaagatcatttaattcgcgagattaaacagattcatgttctaatgcgattattctggtgtgatatcgtgcatggataatagaaagctgatccatttagaaaccaagcttatgcctatccgcacctttaacacacgcatagattagcgctctgcgcgaatcctgcgcgttgcaactgtactgatacaatgcgcaccaaaacaacttatactctagcaatgtacacacatattgcgagccaatctgttcagtttccctttgatatttcaggataatcagatggacgccaaatagattactcttatactgaggaaaatatgaagttcaggttcagcgttacacgcaaatcagcgattaggtctgcctaatatgatttacgtaaataaatctaccaactagaaatccggatattttacaataatcatggcaacgggtatgaccactgggttcgatccatatacctgatgggctcggcaaaagtctgtaagaattctctacatcccgatcgatgcttctttatttattttacttcataaactcgtatttaagctatgcattgccaacagggcttaaataagaaaaagtgttgcacacagaagttgctatgccgcaatggaaagagtactttcatgaaaatacgtagatatttaggagctttcatttagtaggtcatctggttgaccatatactaatcggatacttgcgaattattgtcctttcagcagtgaatcctgagactgataagccagcaggcgggaatcgtattagtaaaatttaaggacatctgagtacgggcgaaatctacaacacgacgaaatcatcaatctattatgacataagtattggacagtacgtctgactgggaaacatagctttatgttggatatgtacattagtgcaaatctgtgttacgtgttaaatcatcgcgttctagaactcttaatcacatagcgagctaccttggcgaacactcgttactgttctcgttttgctatcatgtcctaaaagcggcaaaagttattactgcaggaccgaaaaatatgaaaaacttattttttcatgggactacacaaatcgagttgagcctttaagcggttctatgttacttgagtatcttgaacttggaggggggttataatgataatagcaatacataggttatgataaactgtcctgttttagatacacgggagccttagtaggcttattttaatagtgtagttgttgatatgaataatatagaaaggccatggaggagaagtgctatgttaagagggcagtcgcggtcacgtgtgccattgacgctcacttatatgctgcgttttcgcagtgtctcaaagattaaattagccatatggtgtctattgttttcgtaaacgcctagcatgcgttcgtc

SEQ ID NO: 280 - DNA, 2000 bp, Artificial Sequence
  Exemplary stuffer sequence
cttgtgcgtcgaaatcgaaactcaaatagtatgtacgctgaaaataataaagcctagctaacaatccatccgcgtttagatcgtaattcacattttaccgataaaaagttaagtacaacattggaattgttattacttagccagccaataacgcgtcctaattaccaaaaaaaacagactctgaatcatggtagattaattgggtatcgataacattatccaaattcagggggccattcgcttaagaaaagagatgttaacgtactccagcgatctgcggtgttctgactgtaaaaatacgcatacatttcacccatagcagaagacgtaggacgtcttttctaccaggtgtctgtattacataccccatgcatatctaaaaggattctggacgtattttgatttttaccagttgagatagtgtcaaattctgactttcaaatgacaatcgcaaaaatgtatgcgaaggctgatgatcttgtaatcaatactggtgctagtcacatactgttgtagatacgccagatttacactatacacagtgaacaaggtcatgtcaataacaactatttttgtttataatcactaaccctgcatatgagggtcttgatccaagttcgaatggttgagaattccgagtttattggtaagggaagatgtatcaaatataatccttgcttacttcccaacagtcacaagaagcagagttaacgactgattacggctggaccaataaatattgaaacatcgcaataaaacttgaagaaatttgactacaaagtttaagtgtatacagtagatcggttagggtatactcaattagggcggaacccgcattcctgtcgataagctagtagtaggtggttttcaggttggtatcaaccatcaatattcgacatacattaatccagtgaataggggcgtccggattttgtaaagcattaaccttctgtataaatactgccaatcatatggcttgagtaaccgtttttgtcagtggaatcgtcccctcgctagaagcatctgtacgatatctaatggctgtagttgccttaaatcggaaaggtaagtcggaacctgggctctcattcgaataagaccaatcctaaacggcgaattcctttatcttgttaactgctgtgtcaagtcctcttatcgaaaattcttacatgtttactcttgcgattaactatggtgaactaatcccaacaatgactgttcgtaatagatgtgtttgtaaaattagtattttggtgacatctctagtcatttcatgccttcatagatcatcggtatttcgcaataatctgctcatactatgtacagaaataccactaccttctgacacccttgctagcactctggaactaaataactcatagacgaaaatacaatgcaaagctcatcttcttttgaatattgagcgaagtagattgttgacgttaagaaatgagtagtttcattcgagaacatccgtaatcaactacaattataatctcacaagatcggtctattaaatcgctcatactcctaggactagaaccaacgatcgaatttgtgctttgggcttaggtaaagacgtataatcctacctagaagttatccatttatccacttgataacatatgtctattccccaatcataataagacgtagaagaaaacgactctcacaacgacagtatgccctaatatgcgatggcgactgaaaatcttacggcgcccgcctcaatcacgttcacgtgacccagcacattagatccaggactgactcaagatcattactcggcgatcaacgcactatcctcaattggctatgtgcgaactcctcgtataggataaggatattccggtctccgtatacgctaggctcagtaacgcgtcttactctgggtcaagggtttaaagatcatagcggtatcatacaaaaaatcatatggcctactttgtcgttttaagcgaagatcaacgacgtaatagctaacttaatgagcaagattt

SEQ ID NO: 281 - DNA, 2000 bp, Artificial Sequence
  Exemplary stuffer sequence
tcgataggacagataagtgaccgcttgttgagtcttatatgtattggacttaacatcgagcaacagtctgtaacatatgtcactacgtgattgaaggccgtcgtcagtaattaaggataaggcggtaagacataagataccgtacaaggatatttatcgttatctcaaggtcaaatctaactataggtaacaattaccttctactagtaggggaattccgttggatagctagtaaaagattgcttcaactaatccaacaaagtattacatcaaaacagattggttatcaagattggagcttcagaactagagtggtgagcaaagcactctcatgccttttgtaagaaccgggaatgaaccgcaagaatcacttgacaaaggtattgggtggttatgttgccgggaagctacgattatatccaataggctacggtcgttgtacaaccggttgtctatctggtacttggttgatgacctaggtgcgagccattctgccaaatttatatggagattaagagtggtctttgcctgatgaaagggccaactgccgaagtactttggagcagtgttgactgcagctccaaacatcttgtattttaatatttcggaatagacatctatcgttagtgaggaaagaatttgatcccgcgctattttcccgacattctcaacacttggattacttaactcatagaattttctacctattatattataacaaaaaggtcagtattggtcctgacgtatctgattcacgtattacggggcggggtggaaaaacttggtttcctagagccttagacgagcgttaatatacaacaaactagtttcacataatattacgtatggagtagactcaaacaatggatcgcggcgacgtggatggtattatcgcatgatgcaattctaacgatgaatttgtgtccgcgctgttgtcgttttaacaacgattttgaggttatgatagttataatcattagaacatgtccgaaattcaagtggttcaccttagctttgtcaattttgtcacacttcagggagggtccaggaggaactgcaatcgtcagtctgaatcgttcgagcagtagaaatgacctaatttgctcgtgacgtactgacgataccaaatcaatgattgagttcgaggatctgatgtttggagcttgcgttggacgatctgatactcaaaagtcgacactcaacattttttgccacgacagatattctccagacttaagaaatccttgctgaatatcaaacatgcagcttagattagttattatgtaaattgtgagatactatgctaactcgatagtgaggtgttggtctgacaccgtgaattaataggtcgtccttaacaagtaccacttagattcctcgcttttgagtctttgacgcctttggccggatgcatgtataaatccttttcaaaaggctgttcattcccatccaagttctgtaataggtctatctttacttctggtaacaagagggagttgggttacgacgagtaattgttgtagcaaggataaactgctatttttgattaacagcctcacatataatacgggcagccaagtcagcctgccggcaaatttagcagtgtttctgctcgccaatgtctcgagactcctagctctctcgtccattgctgactagaactagccaattcggcgagcattagagtgctaaaaaaatcggtacaggagcctaagggtatccgggcagaagcaagtggtgccaaagacagttagtttatgagcttacgtccaatgatagaatttgcaaacggtatggttaccttcttttctgtatcttctcaatgtaatatgttaatgaacacattgttaatgtggtttcatatagtaaagtagaaaactagccgacaaccaaagtaagaggagcagttttagaatcaaatacaccaacttaaaaatttgcatctatgtttttgacaattgacatacgacataataaaagtaggatagttgtagatcgtc

SEQ ID NO: 282 - DNA, 2000 bp, Artificial Sequence
  Exemplary stuffer sequence
acaacaatccagaattaaagagtcaatgattaaagtctctataattcttggtggttaaggtgcaacttttgtcaagccaatgcttctctagcttacgaaaggaactagtattacaatttgttaccgcatatactaatgatcaaacattgtacaggtacggttaataggcgcactagtaacaccgtcaattattatcctcgtccgacctgagaaaggatgatagatcgtgcatagagggacttgtggaacgaagaacatttcctacgcagctacaaaagatatattgcaccagggacgtcacactaaagatgtatactacagcattgtttctcataacctctaggtaggtctgtagattcagcgtatatcgactacctacatctcgtctgatattcatctatcgccttaaaattgtgtaaaataatctgaggtcatcaatggttttgtttttacattatgtaaggtccgtaatggtaacttgtgaaccgacatagttccccgtcgcttaggtgtgcagataattagatccaatggatcaattctcggagatagtcttctacggcattctatctgtacacgtattggtacgggggtcgtaggcagggagacatctacaaaagttagcggttgctgaattattaatatacagctttacgcttatacggttgactacaaaaaaattacaagattcttcatgagattgtacctgtcaacttaattcgtatcaaaaattctaaagtgcgcatctaacttcatacaacggagaaaagtacatataagtagggtgtgaacgcagataacgttcaaaatgatttaaactatgattgagatgtccaagttaaggacggtagggttgctaccgtggactataaaccctaatgcctaaatctttatattcgggaattgtttcgggttagggggaatacgcacgaggctaacacaatatgcatagtgcgtatcattagcgtatggaggacgaaaagagatatacccaattatagcctgaatgtcttaatcagacccttatcgtcatctcatttttgactacaatcggtaataactactcgggtttactagatcctaacgggatgactcataatagaacgaatagtgtaaaagcaacctacgcgtaagaccttcccggtcatgaggatgtcatcctatgcaagcgttcctcccgcgaacgccacgtgatctctcgattccattctataggattcattaaagctctactattaccccaattgctgggtgttctaagatctataatgttattgtccagattaagttctcctgcactactcgcgattgtgtctttcgcccgcttgtccccccgtaattggatcgggccttcgcgttctgctaatatttgttacgtcacgtcggataacccctacttgtgcaacatcctgacgaatgttgtaaaaagtttttctttggaaatttgtacagttaaaagacaagataatatgattggatggcaagtgactgtaaagttctatccagtgtttcgtatacgattaatgaaactaaacgagaaactttgctgacctccacccaagatagccttcactctttcactaactccacggtgaattttttttagtaattttcataaaggcaaagactaagtttacctagtaacgccaatccccccaccatagtacactgtgattcgaaaaaaggatatttttgagcttctatgctttagggatatttagtttaacggaaagcaccgtcagcttggaatattaaacacgcacatgatttatggacccatagttgacatcaaggtctttgataccgacggttttcgtattttccagtgaaagccgaagctttacaaaggagagagtaattgagcaaatttctcactgcatgtcacagggactgataaattagtccaaaaactttattacgtttgaccttagaggtaccctaatgcggcttattatttggaggccagactattgcgcgtaacaggctgtttgagcatcggt

SEQ ID NO: 283 - DNA, 2000 bp, Artificial Sequence
  Exemplary stuffer sequence
ctcctcgagcttatagaaaagtcaacgaatgtgtagaaccaagaaagtgaccagctatcaaataaataacaagtgagaggtacagcgtatctaataggcgaaagtctagctccaggtatcggtgaagtctaactatgaattaaacgcattgcgtagctacatggttttacacgcaccattaacaggcgcataactactgcctgaatcgctctgatattaaagtcaaaggaagctaaagacttgctatatcgttgcatggtgttaagtaaatacgactcgagtattttaaaaaatcctctgaatcgaccaactatttattcgttcattctctgtcattgagtagcgctaatcaatgtagtatttggatcaataaccctctgggttaggcgactacatgagtacccttggaaaaactctggtcgagcaaaacaagacacatggggttaaataaagtctatacagtttataattatgcaaatttgacgaattttgtacagaattttatctataatcttacgggggtatacatatgacagctttccggtgttacaatactccttgtgctttgtacacttggcggaaaattcaccacaatgtatggggttccgcgcaagctctctttttcggtaatctgggattccttttttgtgcccttttacataacaagacgaattggtctcctttttactcagaaagaattataatacttttcttacttgtccgtttcccctcatctttttttacctccaaatccgattcatcgccttaagtccagtgtcttccaatgtagtggtttaacgcgagctacataaccatcccggatgtatacgattctacagcgtcttgaaaatattatgtttaggtttcgggtgaaacgcacctagaaattatagcaataataatcttaaatctcctcatcataatagataggttattgataggcgacatgaaacccagcggattcacctatcaccaatcaaaccacagttccttttgatgcagtcattcctacaggcatcctattaacaaacaagcgtgtgccgatgaagaattcgtatctgttaagcatccgacggcacatgtgcaagagtcgatctcctgataccaattttagtacttctcctctgattaaaacaacttccaaagttccaacagatggagtatagataatcaagtttccagaattaatcagtaatttgacaagtggaagcgctagaggactattcccggtaatactataacaagtaatagtgaccttgtgtataaatagacgttgatagatatatatacacttcttgatagctgaggtagacgttgatacaacccgcaagtgagtccattaccttaggccctacgaacatgctcaaacccttttatgctttcccagactcaaaatcaatacgtagatatattgtaaccgtatagaaaagagcttctgttggatacagtggtataacagctcatgttcaaggtttatacggtatgacaaatgtgattttcttttatgtgagataaccgaaccaatttcgaaagattactactagttgaaataccaattttaaaggtatcctttccattagaccccttatattattctactgtattagcaaattttagaaagttcgtgtggtactcaaatccgatgaaactattcaccgtgaccattaaataagtttgatgatcaccgagaattcacacctcgtaaataacacctatcttaatagaattcgtgcgcagctctaagagagagcatcttccaaaacgaagagctgtttacaattgctgccacgtctttgatatacactcttttattgtccaatccgatgtttcacaataggatccatggttccggttacttcctagctaaaagggtttgcccacgcggtgagggaagtctgtcggtatattagacgtagtgttcacgaataagtaagatttttaatttggaatggtttgcaacaattacataaggataagtaaacgcgccgtataatgctcta

SEQ ID NO: 284 - DNA, 2000 bp, Artificial Sequence
  Exemplary stuffer sequence
atctcatagataactctatgaggagttaacgcctagaaattttggtctgcatggtacagttacatatcgtatgaattcgtctaacatttgaacggaccacaccatctgatccgcactcaatggacagtaggcattcggttacactttcgtctggaagaacagtccgaatatgaaaatatgcttagatgattccaagttaatttcgtctataaataagtagcttttgctctataaagataacctcctacagtcgtaacagagctcatatacgataagaagagtatacttttagtttttcgcacatttagccattcaatcgagaacatagacgcctcgagccgaattgcttagcacattttcctaataaatgtattcgaatatccaaaatgaacttgcatgactccgtagcacgcactagatttagtgtgcctaaagattaatatcccaaggttgggctagaactaaaaacgctgttgccaataggttagattgtaaactggcccttaacaagctgattatcaggtgctttggatacttagcacatacttaacacatcggcgtgaataagtgggaaaatgtgcacaaactcattagaaattctgtgattgggtctttacgttatgttaaagttggtattgcttataataacttattctcgcagcgtactcgagaacgtttgaattcgtgagagcccttaaatcaacgacccccggcgtttagaaacggcaatccatatacctgtcataaattatcttagaattattattataccctagccttagccattttgtttaccagaacacggatggatctagttacgattcatataaagtgagagaggctagtgttgtaagggagtgagagagcttgcatcttacgagctcttagctcctcttatcaaaatatcatttgggcccaacaacgcgtaagtcagatgatctattagcagtttggatatgttcaagaagtcctccagcgggtttgcgagattctctgtatcgttgacttgtgacatatgatttgtattccaagacggtcagttgcaatcttgcctgaactagttggattatcagccaccccaggctgttgcatctaattaagttttcctatctgtaaaacctttcacttagcaatggcttaatgctcttaccgatcagctggaagccggtagtactgtcacttggttttcttaacctatcaaaacggaaacaagccgtatttttgatggtagcacttcaaatggtgggcaaccgactaaagaacgtcactctttaaattctcataagttaaaatcggatgtcgagtcaatattttgtcgggccatgggaaagagagcagtatgctaccttcttaatctctaccttactttagacaagcatacgtcaacaactgtgactcttcaaggacgggtattccctgactcaatgctttggaagaacatttaactgggttccattatagtggtcggactctttatgcttatgtcgcaccaggtccatctatcgaattcctgtattctataaacaccggctgcactctaagaaagatcgagcttctgattccaaaagtctataaatgatcagttagcctagcgccgacacattgctccgttagaagcttgacgtttgttattatgagggatcacagattaccgtgtgtcgattggtggctcacttatctatgagccagtttcgttatggtcatacctttaattaagggaacatcgtgctaaaatttttagaatggggtactgtctagactgtctcgaggattcatgccgatgaagacctgaaatttgaatcggaacttttgtggcaccgccgtatcgcaaaatgagaaaaagatatcgttaaccccttataaaccgcaactaactaagtcaaaataagtcgacgtgacttaagatactgattaagaaatggtatcacggctcttttgcaataccattaccaaaattgccaatgaaactgttttggcctatcttaagccacgaataatat

SEQ ID NO: 285 - DNA, 2000 bp, Artificial Sequence
  Exemplary stuffer sequence
attcttaaagtcgattcggtgtcataatagggttatctaacatatgtacaaacgccctataaagttattatcggactggtgcataagtaacagttcgctataaagttaaatgctatcaagagaaataaggcatactgtgatgaaaacgaggtcgtacagaaacacctgcaggaattaatctgccgtatcatacaaggaatatcgttggagtcaagatgactgcccatttgcagttgtcatcttaactgatgatggtttcttgcttgatagcacccgcctcagtaaaaacagatggaacactccaatgctagccaactgaaatttaacgttagtaccaaaggcatccaagcagtcccctggctaagttggagtgtggcatcgatataaaatagttaaaaaaacggtctgatgtttcatgcagtcgcaaccacgcatacggttccggttcgcaacgattgatgtggcggtctcagtattttacaagttttaacatgtcggcagccgctaggtagatacctgcaccctgtggtttcgtatatagggaatttcggtgctttaagataaggattactcataggggatattactcgattgcctcgaaaaatgcgatgagtctctatattcaacggtctattacaggctttctattttctcgggacgcctaggagttgaatgatgcacatcattaagctacttatgcggtcttccataccattccaatgtcgtcgaaagaggatgcagtgacaactcaggatactaataattccttgagaactgtctatttcaagcctattctaacataattagttgctagccatataagaaaatatcatcaaacagatagggttgataacagagggtgctgcccgtatagtgaacatcgtaaccgggtttcacatcctagattggtggcctcctactatgtaagatgtagttatactgaatgtggtgttgtgatcaagacgtaggaaaatttatcagatatgccaactagtatcatcctgagttataaagggggtaatttcggacaaaggtgttgtttcaaaaggttcaagccgacgtacccgcacatcaacttatcttgtaatgattcaaggtttatgtagcttgatcaccaagcaacccaagcgagctgtaccagatacgattatgttaataaaggtttggcgtactagacttaacgctaaggtttcgtaatgtaacgcctgcattcacgtcaataatagctcagtatgtgagaagtccgatgctgttaattctaataacgctcccacttgaaggagaaagcgggagtaggtgcgtttgttcagaaaccacttaagcggtttgtttgtacgtacaaaatttgcttttagatgtatagttgtatacataaccatcgtccgaaagtaaccttcatatgaaactcaaaggcattagttgggaagcagtatgtggcgtttgtgacacatcgggattataaaattccaatatatattctaagtagcagttaaatgaactccactatggttaaatacttgtacctatcgttattcgcaattgtgccacttttacatagattgtgaaccggtatatcgcgtggtcaagaccaggcttcaaagctgtagagaactgtttattctttgagtgacatagtatcgagacttgtataaacatggatggtacacaacgttggaaaagccgaaagccaataagatatttaagcattatgcttttatgtcaacactgactttctaaaccacacaccttaaatcagtagaacagcattttgaaggagtggctaaaccatgttgcgtgcaattctccgggctcgtaaaaacgtgtcgtgctaaaggctctaaatctcgcagtaaaggaggccctccaaactaacttaactcattttgacgaactcaagtagcttctattaaattcgtccgaataccatgaagaacgggattcgcatactgcgttcgccgtagtggagctcgttacaaatcaaatggatcgataaacaaacg

SEQ ID NO: 286 - DNA, 2000 bp, Artificial Sequence
  Exemplary stuffer sequence
ttagtatagttaagataatgcgtcgctaaacaacataaagattctttaccgatgagttctcgctggtattcgcttttttagtcttactcgctcaagttatcttgagagatgtggaactgaaccacttgaggtagccccatcaattataaggaaattgaaataggatcgaaatattctgaactatttccatctagtctactgaaattaacattgacacctttcacaaacgaatggcaaaaaaggacggatccatccccacagacaacttcgtttatttcagcacatttgtccctggacaacagccgtatgtggttcgacatactacctgatagtgagcggttatcgaaatgtccttgactagctactaagaggctttatacaatattcctacacacatagacccagtagatatgagttctagttggagatttttcaacacaattacgccacgaggtccgacaacgtatcctccacagttaggaacatttattacaaggaggttagctccgtgctacagcaacacgaattactccaccgtgttgagcaggtaaacgagggcaaaatacaccccaaagcgtaactgcatacgactttccgctcgaagattgttaaaacaagactgcaatttctgtggcaaaagacactaaagatgacagtacagcacccatggagagtttgtacccggttcgacctaagtatctgttgtccagaatcgtgaaatttgaagtggcctaaaagctgagacgagtatagtagggtggaggtttcctatatgttggtcggtcagtaaatatttaaaccacgggagttaaacttatcttaaatgtatctatacattagtatataggctgagattcgatatatatagacgccaccccgagaaatagaaagatagtgattcaaattcctaacagttcggagtggtatacgcatttctgagtaatttggcgtacaaagtttgagtagagcacagagttgataactagagcaatgtctgagagtggattaacttggtgtgctctgctagaaatccccagtgatgatctctcataaaaagtgactgcaagactaggatacaatttattatcgaagtatcaagatcgtgggttccttttttcctggtcaaagatgaatctgtcttacttaacgaaacacaggaacttttcttgcataggcaccgatcttgctatgtattgaagctacttcaaaggacctatcagcgggtgtacacaatgtcggaacatgcataaatggcagaaggcgatgagtcatttcgcacaccaacaggccgacgagcgtaggagcgactcagaacactaccaactatagcataacgataaacggagaacgtccatgccgttatgtgaccattcggttcggagtcgtgggttaccgaccacgatagaacatggcacactgctttctcacttccccaataagaaacaccctggacgtatacctcgattggatctggagacagtactcggatccacacctaagtagtacctcactgtgggcgatggccaagacgcgaggttgactatctgcgtggtggaaaaggccgacagatctttatcaattgtagtgagctgatgagtcctttatccgttataagctacttttattgggtaatagatggtgctcttactccttcgagttaatatatagaaatcaccgcaaagttaaacgcaacatgagtggtttggattaacaacttctggaatcattataaccttaggagcgttctagtgatgctgaaattgagacagtaaaaagtgcccatgatgtaggaaagtcactataaagtgaatctcttgtccttaaacataaagcgcggtaaacactcacgttaagatggttgtggccacaacatgactcttgtggttcttgacgtgttaacgcggtggcactagcagggatgatacaagttgatgcttacccatatgattattgttccccggagccaccactaagccactaaatgaagatttttgcggcga

SEQ ID NO: 287 - DNA, 2000 bp, Artificial Sequence
  Exemplary stuffer sequence
gatgttctgaagttccttagcgtacaaacacaaaacgtgcattggaaaatggagagggaaccctctatgtctgatgattttttcggttgagctaattccagtgcaatcgacaataagggcatgtccgaaattcgctttttaatggtagtaggtccggcatcattatgttgtcggcctaaataccataatcattgctcaaccttcaactctttgctggaacaattagtacttttcgtttgcgcttaaccatgcgtataatgtaataaaagcaccagtttatagatatcggaaaatttagagttcatgccatagtttgaaccgacggtaggtacctataacgtcttttgatttccgcaacctatgtattgtaagcagttgtcctaaggagtattttcactgtctaagtggtaaccagcggcgagaacatagtcggcggaacggttctgatttcgactagcatcggcgacattgccttgtcaatctccataatgatataaacatggtcttttaactctcacaacctaaattattaacaggtcgatacttctctggcgaggttgttttaaaacttccactccggataggaatttcattgaaaatataaaaggttgatgtgtcaatcgaagtctaaaaagaatgaagattagtgtcgcctaggacatctatttgttttaaagtgcaaggaacgtgttcacgtagaattgtgaaattggatacatgtttagtgtcatgcattgtttatgggattgactataacttagatagagaactagttacccttattactttgcagtatatgaacgactgattgtcaagactgagcctaaattaaagtaatcagcacattttggatatggataggagctcagtttctggtttcactctcatcgacttctttgtccaaatacggcaatcacgtaatgcataaaaattcaaacataatgtgatgaaagaacatatcacccgtctaaaaaattaaatatatactatagtgctgcaatacatccttaaattgtcctatattggtaagtcaaacgatacaacctgcattcttgggggataactgatgtttactggacggcggaaatactttaatttataggctactccagtgcatagtaagaatcataatttggtagcgcctagtaaaaagaaatcctcaaaaactaaacgctattctgatcgctatcatcaagaaatgaattgtaagtgagggctgtattctaactcatcctagcaggatttattgcctgcatcatcgacattctgttcgaagcggtgatccccatttggacaaattcaaggtttggattatctagcgcccttggagtctctttacgtgtttaggtgttcctgtaggaaaatcatcttattgtcgcgaatagaaggtacaaaaagacctcaaagttaccatatgcaccatggagatgaaacggtaaaagtaactgggaccaaagctgtccttccgggattcattattaccataatcattaggcatcaataatattctgtgcgatatgttgctcggcttattaacctcaatgaaacaatatgaccgcatatcgctacagtaaatctacgacgtttttactgattgattgaatcgcactttttaataattgtatgccccgatacataaaatgtcataatcgagaagcatatagtagtattgtagtatcctcaggatcggttggtagctttaatacgtgtaaatttttctcgtaattatcgagagtgtggagacgtccgtgtactggattcgtaagaattcaataccctgatgtccgtccgagtagatcgataaagtaagtagggatattcagatatttaatgtatttcctgtacactgtgacatctctgcaacgagattgttatactggcggcgcgtaggaaaaattcaaccagtctgtttgcagggatagttaaaattcattagagaccagagcaaataatgagcatccgaaatgtatccaaagcgatatacgcgcttacaaactctg

SEQ ID NO: 288 - DNA, 2000 bp, Artificial Sequence
  Exemplary stuffer sequence
ttgatgtgcgaatataacattgatcatcagaggcaaggtgataggtattaaaacgttagcgtccacgctcctggttctataaaacttctttagatgctgctaagtccattgatttactgttttatagatacgagagtaaatatagtttaaattttttaagtttgaaatacgtgtagctatcgttgcgctaaggagagttgtctatgtactagtgatttcagtcggaaatagcagaaacatgaacctatcacatgactgtcgaatggaaaatttggagtctggaacattcagtatgagatatacattaatccatgactcagaggaattgacccactaatgttattcttagttgcaattccaggtatgtctagaatttgcaatcggttagccgttgtgtacttcgtatcaattttcaaacagaatacaaaaccacgctagttagccgaaattactcctaattgtcgtcactatgtaagagatttagaaaaaatagtatttggtactactaagataatcgctgtccactataaacttgtaggtagttagtcgagtgttctgcaagggtacattcatggaattcgcgagcaacgttcgcttctccccaaatattgatataaagacgatccattctatgtattttcgcactagtaaaatacctatctactcgacttacgctatagctcagggatctatttgtaggcatccacagctcagacgaaataatagatttacgaactgatagcggccctccatgcctgctaatcatgttcatacatccaaacaaatcgttttgttggtagacaacaacatagcgataatttcaactggttgaaatggttgtatagctgaatataaacgatcccaaaaaattcaagatggtggctgcaccggaacgacgttaatagcgtgaggaggtgttaaaagcaacaaaatcacacccgccgtcttctagggtaagcgggtgccagccgggtctactggataagtagatatttagcaaagaacctcagttatccattttctggttacgtgcacaattagttttgcatctgccggcttttgtctctggcacttgacaaacctagcaaaactcaactgaggggttaacacgctctaagattcctcttactagatgaggtattcatctgcgtatctgattctacgttataggctttttctctcgaatactaatgtctggactgatcaataagaattggctaattgcggaagtcaaaatagaaccaattatattcatacttctattattagttctaggatgattttcccgaccatcggtagtaggaggaggtgatgtaactcagtagtattatgctgagtgattgcacctctgattctattaatatggggggatgctgcttgcctcgtgggttagtgtccggatgaaaacccccctaacctattcacgtatagtatcccagtcaattgagtcagtgaccttaatcctaacaaaaaatacagaatgctgtgaatgacctcgttcttcttattgtgcacgatctgattcgaaaatgaacggtatagagtctgagcatcacgatataagagattcattctgtattatttacgaaaggcgtagcaccattcgatcagcgagcagaaccacggggcagtattgaatttccgtttttccgatttcaaaacggctagaaatggctgctggatgatagatgcccaactcacacggttgaacttgcttatcaattgtgcggttcatatcagacatagcagtctgcttggaagatattgagtaacttcagcattcaaacgcgcaaagctattgagttgcccctgatgctgtctatcgtgtattaagtgatcgtgggaattagacatacaactttacctcttctagcttgtttatagagcctcaccgaggtataaatcattaattacccaggagaccggttttgctattaccttgtaatgttcaaaaaagaagtggaacacagtgaaagcctcatttctcaagcaagtgagta

SEQ ID NO: 289 - DNA, 2000 bp, Artificial Sequence
  Exemplary stuffer sequence
tgtagacatttgtcttcaatctaacctctttctcacgaaataagggcttgtattgttccttcgtttgtttaccgcacagaaacagcttcacttaacatacattgtaagtgtgtatttctcggggtacgtaacataacgaaacttaaagcaatcagacatacagtgccattccctacggtactgtctcagtatgttaatactactcatttgcaaaaggatgtacgcacttcatactacagctgctgacggtgtatatcaaacaattatattaacgctcgtaggatagttcacgtccgccatatctttgatttaggcttcaaaattcagaataatacgaaatagtctgtctactaggccaaagtcacttaagggctaagagtgtaatgagtaatcaaaataataatcgttgagtcgtcaattggagcatcagttatggcattaaaacatctagtgggtcgaaaggatcaggaaattatgtatgggtgagagtcgctgctacggtatcgcttttggattgagggctactacactcagtacccacagtgtgtgtattaataagaatcgcaatatgcgtccttttaagttttaaggtaccctacctttcatatctagtggaaatcatttacgcctatgcgacaaattagagacttttatttgtaaaacattggatgttggaatgaccctagatgcatgttaaatagcacgttcattagtggtacacgcctatcactaacgctatggaaaaatagaagaagccagaacaagtaaacctatggtgacaaataattacataaggaaatccctcataattagaataccataaaacgttagttgtactatccgtaatctaccttctagcgtggaatagttgagtgtattctagtcacgccccgttccataacgatacatgtaaaatttacagcgacgtttaggaaccctacaaggggagcagcagcgaggatagctgactagccttacaataagcacccatacttatgattgacatgatggtcatgcggcgttaccactccgctagcgttacttctttcgtcttgtaccggtttggcaatgcgatgcagcccaggtaccgtagagaaagtagcgatgtgtgaggtcgagtactttgtcagaaagcaagtcggattgcggtcccatttaccgcgacgtgcatttgtacagtatgaccgttttttaccacttactgatgaggccagactaataaacgatatttggtcacaggacaatattacggccaattatgaaataactgactggcctattgaatgactaggaatgtcaagtccagactctagctatttgggaggtttatatgtttggaccgacttgtgggagtttgacactacgagtaacaagattatccctttttatgctgcgctagttgacatggattgacgaggttattaatatccatgactaactcatcacagcttcccgagccgagacggattattttaatctcgttgatcgatatattaggtgacgtgagaagaagatgtgtcgtaatcagtaatagttaggatcaagaggttaaaagaagcgccttcttcacagattctcagtatctaccagcacagagttctcagtttctaacgtgttccgtatggatttgcgccactttctgaataagtcttatgagatatacttacctggtccagatgtagcagcgagttaagattataactgcggtttagcacgcagcgtttaaatacaaatactcttgactgttataacgttcaggattaggaacaggttcctcacggatatagaacccaattcacgtgcatgaggtattctatcttagggggaggaactgcgctggagcttgaaactgaccctctaggcgcttgctttcactgagatctattcaaactgacgtttagtaagaaatcataagacttatctacgccgccttataatttatgttattaaaacatgatcatgcgatcaattaggtaaatttctttgtgccttgcaatatg

SEQ ID NO: 290 - DNA, 2000 bp, Artificial Sequence
  Exemplary stuffer sequence
cgaatatttatttttcctacgcacctacactatcgtgaagttcatggtatcaattatatgtcactagagccacaaatacgtacttaaatcatttacctcgactgaaggttgtaggcttggacatactcttgccaccattgtaacaaaggtagatcggttggacccgaaatttggtacttttaatctagaatcagcaatatcctacggaaaggcccaagagatgtctcaatggatgagagtgtaattacctaatttcagaaaagagagtttaacacaaataagaacagacgaatatcaataaagtgcacgtcgggcctaaatgagcccacagcctggatagattaagtgcgatacgtcgctaccaacgaacaaaagtatttggtattatgacatcggctccgacggtataggataggaataactcccaaacaatataatcttggatacgattaagtttgagtttgattgatcccatcaaacatttgttggtataaagttaatgtgtgatccagttagaattatatgaacatagtgttgtcacgattttgagacgaccgttaaacattatactgcggtggcatagcaagttcatctcctgacattagtcagcatttaatagtaagcaggagtactattaacacgctcctataatcggttgcctgttggggataatcagaacatgaaaaactccatattagaaaattacataatatagatcacgtgtatgaaacctaataccgcgaatataattacattatgattgcaatacatagggtagactcctagttaacgtaaaccaaataaccgactcgagaaacacaggactaacaattataatttataaactaagagtgctatactagttactgcctgatacctatgtttatttgcaagtcaaaagtttcaaatagcccttggcaagctacatgatgggtgattggaggtgggactaggagttccgtccttagtctgaataaagaacatgatgtgcaccgatttgtcgtctactcggacgttgtggcaagaataaaagtgaggtatagtaccgctagccgcagagatactgccttcatatgcgccgatactctattgttcataaacagcaatgaggcagagcacataatcttaattattaatttagttaacggcttcccaatttagcaatgaataaattttttgaggtgcatctgtgattaattcacccagaaacgctttcgcgaattacctgtcactatagatccttaatgaattatcttcgtcgtcggaacaattatcggactttattttgcctgttttatgtatcgagttaaataacgggaatcataattttatattacatctgttttgtatagcggatctcagtaggttacatcactgtcgtcggattcaacagcaacaacaccgttaatgaatatagctacactgcatgagtcccaacagcactggtccactagaaatatataattatacgaatactttgctatgttcatgacctgtcaaaggagaaatctagtaaagacccacggatatcgaagaacattgtagttctgactcggtttgaatgtccggtaactgcaggttcccgttatactgagcggtccgaaaatggcagtctaagtccccctacatgacgattgctatttattaggtctcagaatataacattagacacaagagcacaatagtcggagtatgcgttatcgagaccgtatatgagtcaatcgaacgtagatcgatcatagctaactaggtggtgtatcactgacgacttgacgatgttttatcgctgattagtttatgatcttgtaaagattggatgctacatattatggtaattttgctacttcccccaactataccaaatgactcactgtttatcaaaggtgactggataggcgctaggtatatcccggtgcgcaattattgccctggcgagccgaacatctcgaatatgtaaagacgaatactccctaattaccttttcgaggtaacaatgaata

SEQ ID NO: 291 - DNA, 2000 bp, Artificial Sequence
  Exemplary stuffer sequence
atcgagttggtttctacgagtagctggcaagcgcacatagaacacacattgcatgtgagtggagcgattgcgagacgaaacaaccttccaaaagcccaacgattacagtgctagttatctatgggaacttattcccttagggccaaagtccctaggttattctatacgactcacaccgaagaggctgtaaattaacccgaatatagatgattagtcctttgtttgtcttagggatggcaccataataaaattgtcaaattagggtacaggactagttcgatttcttctatccgtcgtcctaggtttatatgtggccgtcaccactgtatcacatgccagctagcaacagtatgatgtatagcggcaaatcattcgtcgggggcatgcagaacgtcagttaactttaaagatgagactacgttttggtcacaatacaatgacttagactcatctcttaactcagacaatcacttttatacttagtgcaatgtgtcacagccacttaatggcctagctaatccttatagtcggtagctagcgagttatagaatcttgttgtggataatcctgctcaaccttgcctggaagtctaagaccagtactagaagttaggcgtcggagtctgtgatgctaaagttgttcggccaactaattaggggtgtacctccttgtctaatcctcttagaaattattcgagaagggtacagtacccctcacaaagagaatctaagttaccgtctgaagtctgagtgatccgttttgaggtaaacagctgttatacatacttacagcttagtctacatgacctactaagcgcttcgtgctccttaccgtcccagaatacccatggctcgcgtctcctgccgtacaatacgtagatttaatactcgtaatgtttacaaaaaatggctcagcgaatatgaatacgatatacagtaccatatttatggatacaaaatttgtggcatccgcctaatagggctttcctcagggcttactccacatactgttcaaccttctaggttcagtaaaagtggagaccacgatgcagtgtccttcttaatctggccttatttgtcgatcccttatctcgctaagattagtcacacgacaaagaggtcgttaatgacgtatctagccacaatcgacagtcttctggcgaagatatctacaagagtcgttgattcgtcacttttagccttgtaaaattgccctttgaataggtgacacccgaatggattggtactttcgtaattaaccgagactttggagaattgtctccggcgtttcatgtggcgaagaatagaggtgactttgatggcaccagaatctcactgacaattgctatagacctaatatcggatatttctgcaacttcctaatcgaaaaaatttctacaaaccagtcgcagccttgagtattcgcccttgacatagattcacaagattgagtcgcaaatggtcctatgataatggatgtgttattgctggaactttatcatgatgcaaagaggttataatattttgtgttagtagcacacttaatgcacgcagaatccttaatcaatcattagctgctaatgagaatcaaccgaccgtgttggtgttactggaattatattcagtatcgctctgatcttaaggccctcagcacctgaggtctaacgaaaatttttttaagcccattctcgcaaggccacaaccatcagtctctcgagaacgacattggacctcatatccaagcctccggttattcaccgatgtatttcttcgagtatctaaaatctgccaatacgattcaagagaagttagtatgcgggatcatgtagcgtacctttatatgaataaaacatacctggtagatggaaacttggtgacccgggagtacgtcattctggtactgatacttgagggtgaacatggtgcgtgattccagtatagcggtgaacctacgacaatatgtgcatggcattgcttatttggtgtatcgttttttgagaa

SEQ ID NO: 292 - DNA, 2000 bp, Artificial Sequence
  Exemplary stuffer sequence
taactatatggtgtctgtttactacgattgcattaagatttctagcaatcttctccagtaactgcacttccccatattgtagaagcgacttatggagctaatctttcacttggtttaatgctaactgggatttgagcacgtaaaacttaactcggaccactttgttgacataattccgctgcttatatacccatattcatgtctacgattataaagttcttcgtatttggctaagcgtctctacctaggctcaagcctttttagccaatctgaacgctaaacgggtgctagcctagtgattatttaatgacgatttgagttcatggacgaaattacattattactgtctaaccggacaacgggcacgtcacaataagaagggtacagttgggatcgcagtttattcatgctgtatgccaattctactacctctcgtcatcttaattcatatatagctgaagggctagcaagtagtggatgactataatcgggatttagaagagttttttcctcgaacattagccttatgtgtctattttgttaaaattgacatgctaaacgatagctattagctggaggaataacataatgttgtaaaaggtaaccagctcatcacttcaggaatcttacttcctacgatggctgtcttttagtcgacgtaaagaaacccaaccaaggaatacttagacagacaggagatcatcctacaaagatagtcgatcttttatttagtccaacgcttaccaatgaatagggctgtctgagactcaaaatattggaccatgggtttcgcaaagcgcaaacggagaactatgatttcttgttgtggcagcgtatggtccccacgggtgactgtacaatcacggagacttttatcatataacgatagtacatttatctggataccggatccttcatttctcggaactctatacttactttaatttaatggcccgaaatctattatccttaaattacaccgccgtggactcggaatgaagatgagtccgcaaggcatactgttagatcggctgagatattgcctagtgcaatcgatcttttgatggtatttgtgtacattctaattcgaggcgaaactgtcaataaactaatgggaaaagcaagcatatcacgagaaatattctaggggataacattacgttttcggaacacaacaggttcgacataaatcttttatcatattatttgcttacaattatttagggcttccgcccatactcagtagttcaaatgatgcaaaggatgtggtgtctagtagatctcttaaatttctatcgaatggcgtagttacattgcagttatttttacatggcaaaatgatcaaatttgtacgcaatagcagtaacatattctctgtagtctatatctttatgattggagactgttaaaagctgatatgactaatcaagaaaatatcgaaatttgatctacgacttaacattttaactaagcagacatcataacgtttattcttcaacgggccgttactgctaaacattaatctaacgtaaatcggaactctgcagagtgcccgtctcttattttgtctgaattttagaatttacaaggagatgctcaagccgagttagaagaagagaaatataatgaatccaccgagtgtatgtttatacataaagaactatctttaggcgacgtgctagatcccactatgttcatgtgtaacgcatttattggtggaactctcgcaaaatcttacattatttcgccattacgtctatacaaaagctagatccgtgaagggtcataacctcctttaaaggcatgaaagaggttatctaacttatgattctataacatcgtcactggtggagtaaaaacatctgtgataaatacttgtgatactctctaacatccctgtaatatgatgatcataacgcttgcacctattaacttaaaagaaagttgtcttatggtgattcttaaataaaagtgcctgagccaccttgtgtaatttttaa

SEQ ID NO: 293 - DNA, 2000 bp, Artificial Sequence
  Exemplary stuffer sequence
cacaatagtatagggacgtctattattgaaaattataccatgtggacatattctggatttgaatttattttttacgaacttactcgtctctttgtcgaactgatcgaaccatgataggcggtccatacgtgtagtgtgtgctagaagcatctgtacttgtattgaaaggaacaaagtcaaccatgctgttcaccaatttgatacgaaggaatgtcctatctaaccgggcttattttacaggctaagtaggtgaataatgacaggaaaaattcgaataaatcagaagagttttaagtaaggctcactggtcgaacggtgataatactggcggcaagttctatgtagcttattagataactcttcgggtgagagaaagagcttataaatgtggcgctgaaatccgatgccagctgtagccgagtcgcgtcatctcctaacggatcagttaacattatgcttactggacgtaaagtggcttgtctagctctcatgcgccttgtaaagctttttctcactgtgttcgattatagtgctctcagcctaccgttgcaaacaatgactagcgactgagatgacaacacgccacacatatcgagtggtaccgtattgggagggtagtggagagaccacccgatatggataacacgtacaagatgtggttaaagagccaatcacaaattgagcggcgatcgtgtcgacaatttttcattgtgtaagcatgcatgtatactagaaatagagtaatacttagcatatacgattaactcttggtgagatgagattctagctttaaaagaggggataccgatagagtaatacatgttcttttgagcaaatgggttgttcgccctgatccatgataacgactatttcatagctctaatttagatgcttgacccagtgtaaagatccgttttaactaacttagatgataatgagaaataaagtaattgactacttagtacactttaaatcctccagtcgatgtgtattgtcgctatatcgcaacccgatgttcacatacagggtcctgactttgggtataccttagtacgtaacaatctcactcacaatcaatccaagcgcggttactatgttacgacggggaagcaatacacagctaggcgtgcagtactgctcttagctctccgaaatctgatctagatgcccaaataattttgtttccaaagctagcgaggttttacgaccagtcatgacagattctgcagttgaagcatgtcacaggtaagcaaaagcgtggaacggatggagcgagtaatcaatagaacttactttacgagcggtgttacaaaattgggtataatgcactagccgacatcgatggtgtagtgaattggactggcaccctcaaggcctcgcccaactcagtctcgctagtttgctacctgcatcctatgaagctgtttttaaaaatatcgatttctagcggtagttaaactattaggaagggctaaaacaaagttaattatacttatgtgaacttacaatttatatattagaaagtgagtaagcatatctgaacaagcatcatcgtaatgaggtcggttcgaagtataaacttaagttaacgacatcttccaataccatcgaagtctactaagtaagttaggtgcttaatgatcattcatagtgtagcaagtccccgcaactagataaagtcaacgacttaggagtttagatagaattgtgtaccactagctcgctacaattggtttgtctagacttaatcccttacctgttgagaccgactctatttcggtaaaaatcggcaaaatacggtaacattgtctgcagtctgaacacagactagcttatatacatggatcaaccatcaggtgtgactatgttttattatatgaactgttaccatggcgcctacgacaatagtatatttccatttcggttaccagtttttgtctactttatccattaagtgatatatatacatgtgtccaacgttatatggacagcgttgtgca

SEQ ID NO: 294 - DNA, 2000 bp, Artificial Sequence
  Exemplary stuffer sequence
taaaagaacggacatggcgcacaaaatgactatgaggcggttacttctgatgatcacaccctagttcttactcaggctattgtacaccctgccctctcaatatacccggaaatatgcatttatacggcaatcgatcttgaatcccagttcgagtctttacaaattccatcgtttactacgcaacgtcatgctaaataacaccttcccatatatgtagcgtgggcgggactattagagtcactttgtgctaaacagccggtaagtataatagtttactccggaaggtgtcaatatgtttagcgactgtattttggtactttatccctaaacttagctaatttacacatatagcagctggaggagcaaggtatcatttaatcttgcttaagaccctagtttgtacccctgtcgcacactaaacccaaaattgcgacattgagccacttaggccacattcgttaatctggtagttacagcacaatggctataatatacagatacgtctagaaaaaagttatttaatgcatagcttgcataatcgattctttaaaacagggtggggagctacgtatctaggattttattctacgtcatgataacgaatcttcctgaacgtactagatggcgactatcggagaatgatttagaacgccgggtgtgtcttgatgatataacaataagtaccacgaaaagaatgtaaataacttgatatcgactgtcacaatttgtttgtatcattgttcgtatcattatgctcctgctcgtgtcgcaattcccctttcaccttttggttctttatacacaatcatattatagacttatacggaatattggttgtaacttagagtaataccgattgaacccacatgtcgctgactgcgacgctacggcatcttaagccgatatatcgtcgtgacgtaactaggagtccgtaagcgaagagtagcatagcgatgatcgtttcagactcggagtattagagttaccatgctagccacatagaacggccttccgtaaccggtggcactcgttcgcagtgggaagcccaagttagaataaattgctaaatctgattctcccgtctggacttcgatcttcgagctagagtgccactacgggcactaacacattcaacgagtttcgtcgggtggctcgactatcggcacgagtgttgctctacgagaatacctgccttccttactgcgatttctctttacgctcttccactggtgccaagtggctgtatattactggtcgagtagggctcgctgattgtcgtgattcaaaaacgcaactctaaaatccatacctttgttgaatacctttattctcgttatcatagaggtgttcgggccctcactatcgatggcagatatagcttctccgctcgtactttcatatagatgttccccaacagctttaaagttagaatgatccactttcagggcatccagtaactcgagcaattatgtatgtaaccgatctttcgatgataggggatagtacaccttaacccttgtccccggtgaattgcggcgacaccatgcggtaggcgtatgtacggtgtgcccttaattaacatcgctactgtactacacggttaggtcgtttgaaaaggcagccatgaatgttaagatcttattttaaaattgatcatttacatttagctgctttgggggtaaatctactgatccaggtattaatctcttttgtataatgtaccaattgtagtaggttctctatgttcttaagtttcattgtcgataataaactaatcggcaaaggaagaaaactcaataacttgtattgtaccaaaaaagcgggggctatagttagatcggtgactcactttcttcgatataagggaaacccaccgtataacgacggtgatcttaagccttctcccaggttaacgtatagcctacaaatgaatgcattcaaaatgtcgtaagccttttacctggaaagcacaaacgatagcgcatttccttaaagtacct

SEQ ID NO: 295 - DNA, 2000 bp, Artificial Sequence
  Exemplary stuffer sequence
acttgcacagaaatgacaaagacgtcgattcacgataaggcattccaataagtataacataatcgtgtttcggggcgcacaaaatagatacccaaaagagtgtcctttccactcgacagtagagctcatagttccgtgagattcttgcctcgtaactagtagactgtctatcgcaagaatatcacacccaatatttaacaacgctctgacgtagtagtggctacttgtgcgaataatctagtttctcatatttgcgattcaacttacggctaaacggcctcatagtttttccctattttgaacataagtcgctgttaagcagagtgatacttcccttatttaagtgtaagatgttaaacactaagctagaacacagtaagcccccgtatcttagacgtaatagccctgttagattaaaggattgcgatcgacataccaacagatgacattaaagcaagtatagcttcaattcccgccacggtaaacacctatcacgatacaaaggatagacttaccgagtaccgtagttagtaacctctaagctagtaaatcaaagttttcgctagttattcataagaacaaaattacaaaatgcgtatttacaactcatttacagtgatgagaccgattctaatccaatcggtgttagttttgcttatctgaaaatactgttagaaatgacgtggctgttaatcaatgtataacgtgcatgcgctgaatatcaatcatcagtatcgaggagttggcatacgcgggggctgttgttaaaaattgatccgaatcatctggtttactccactaatggattaagcctcctcaaggcagctgatgtgaaacccaaagatgtcaatttgatttcggtaattaattgaaatccctgtcctgagcagactataaacagataaccgtatggaaatctgattccttagacgttttcaaatctattcaagtaaatttttacgggaatcttaaacgatatcgttccgtgaagtaattcaaaaaacggtcttgatcttataattcacgtttgatactaatttagtcctccgctccctaatgattttttacgaaatggtccagtttattgtttttaaaactctttggaaaattcgtgtatgaggatgataaattgttcgatcaacgtttgtatacttagatctcaagcaagaactgtcagcgacctgtcgttaggtagtttgttgcctgccacctcgcgaccttaggaaaggaaggtaatctattccttaatacgtactatgtacaagagatgcaagaaaagggcaacatgagaacggttagtctctttgaccctcttactggttagtgaatatttttaccagctgctacgatgcaggatatctggccctttgactgttccatggacacgagcccgaaggatatttatttaatcgagagctgtatttagtatcttcataggacttgaaatcggataccgctgtaattgtggaacctcatgagacctcctaacaaaacaagtatcgacctgccctatctccgacatttactcaactctacccccaggttgacaatttaggatggtgtctatgggaaatatgattcgtaacgtgctgcctcaagaataggttatgaaaatatatatataaaattctatgatagttccttcgtctcactcaatactaagtcgttaagccaactagctcgggcgggctattagttgccatatgaggatccatgaatcaaacaaataatgcaattctgctaaaaagtgtgtatatagagcgtacacacaagaaacaaaactgaccgatccgacttaaccatttcaatataatgctgcacccttgtcctcaatagcttgcagggggcaattacgtttggagtctggttgtggtaatactcgactgtcctcggcgatatagaataattatagagtgtattatagcacaaattattaatagattccatagcctggcgttacatgaatattctcagttaaagcatttgaacgatcaagtggt

SEQ ID NO: 296 - DNA, 2000 bp, Artificial Sequence
  Exemplary stuffer sequence
aggaacaatgttaatatcaagtcgggtccaaaaagatgtgtaaagtttgcgaaccgttgcgatctgtttctgtatcgtcttacactgtcagggcactaggactcactacgactcatatgtacattgtttagctcactccgagacgcttagtgaatcgttaataggttgatttgttattgaagctgtctgacttattatcttcttaaacgactttttacgtattgggagtcataggcgttttacagatatccgcgtcagtccacgacgtggtgctctatcggataggtacaatcaacaagaatgattattgctcatcttaatttactatgtgcgccgtttcaccccaaattcgctcaagctcagaccattgagggcggaataggattgaggggtagtgaggcgctgctgtattaggcaaccccggtggttcatttgaaaaaacaatcgcggaaacaactctaggcctaaggggaacaatcgctttgactatgagcttctatacctttgaatatacactttgcgtggagcttggcgcgactccttttgaggtaatgcgatcctacccattttgggttccctcttaattatattatcggcttttgtcaccatgatctcataatactgataagttacccctgatgttacgaccccgcagccgttagatattttatttaggaggacctacccaaggcctatgatcctttctctatatcacgaggattacagacaagagatgtgtaatccgcccaagttactctactcaaggttgcgcatattaggggagggcgtttgacagttgcagtatgccatcttggaaggcaacaataaacggtacacaactttacaaatattccataattgtttctacttttcattcattcattatgtatccctctatacttataaaacatgtacgacatgtcctgtagagcgggacctgttcccgctcatgacagacgagttatttgtctccgacgtatcatccatctttaaatattgaatagcagcagcatcaagtgtggataagtgcaagcactattaaatccgcgtgaactttcatatgacatgagaatcggactgtctgttatcgtaaataaacccgagataatgttaaaactattctaatgacttcatgaagcaggatcatctaaagttatcacaagaggtggtcttgagtcttgcaaacttcagaaaacatttacaaacgattcaaattagcctaaaccacttacttaaccactcatattccacaagttacggttctttagaatattaaggtgtaatgacccatcgagccttatagctcgaatcaagattaaaagaatattctaaatgaccataccggttacatgtgtgggcggagtcaaaagtttttctgactattaggtgcacaaaggtgttcagaacttaaccaaactcttagcacatttgattagctagtcagattaaggtctccactttcttttctgtggtagttcggtaaattgatgggcattaacaaacttaaggttgattacaatggggggttatcggatggttattgtaattgacccgtccatagatttgcttaaaaatcgcattttgaatacatatcctaacttccaagcattacacagcgctgcactatagagctaggatgactgtacaacctcggattatagcttctacgtaaggcgtggccgtggctggtataatagtggggtggagggagaattgacaaaaaaagtttatcatttaaatattagtaatggggttgtcgttctaggaccgtatttcgcgtactaagtcacatacccttatatattttccacagcaagtctatcattgcaagctgttaacttcattccggcggctgctgaaccagtatcagttggtccacagaagctaaagttagcaaagtaatacacgccaacctacttatatatgtatatcgtatagcttaattgagatgtcgtagccattacatgctgagccttatttttgaccgagaccaggtacac

SEQ ID NO: 297 - DNA, 2000 bp, Artificial Sequence
  Exemplary stuffer sequence
ttggacgtcgaaattatttttgatatacgtgtaatgatagactaaaggcaaaaagaaggagtataagtctaagttcgaagaggcggatttggttatacgtcctgcacctcttgccagacattcttttaattcttgtgacctggacttgaagttcctttttgcgaccatttgtgggtttagtacgaaacccccataagcagttagcattaaaccatcaggtttgactcgccacattcgctatcgcaaatgctactaattcatcttaatctgacccccccgggaaggaagccatttaatagataatctgagtcgttccagagatgtacttctcagataaaccgtgaacactattacgacatatgctgaataaccagtatgtatggctgttgtcgactctcattcctatagtggagagaactgatacatacatattccctacacggatgttaaagagtcgcaggacctggtgaggcactggatcaacaagttgccaaactgagtgccagtggagctaatcacaccttcggctctgcgttacatgcgttagtgaaggtccttgaggtgtgccagcaaagattgttaacatataatctaagggattatatggtgtatatgggactgaaaacctagaggtctgtggggaaagaccgtacagtccctgaccatcacaataaaaaatagccaaaatagcgtgccattctaaaattttaatttttaatcaatcgcgactcctttggtttcatgctagttgattctatttaagaatccaagtgagttttaatcttaaccctaatgatttaaggttccagtaagcaaataaacgactcgccgtaaagcgaaattgatcgatacgtttcttgctttatttttgggtacagcaatccttcgaaatgttggcttcgtaattccctccagtaacttaaatcagttaatttgcattgtaagaaaacagcaagtgaatcatgtcgccgcttcagtaacttactgcaaaatgaaagcctaataaatagttacccatctatctaagtataaacgacttttgcttatgtccacccatgctaggctgtgaatcctcttacgtataacgtgctttgcgtgtactttcgaactttctaagtatcaatcgcaaatcgaagtaacttaccaccgctcgtaggaattgcatgttaaaaagggttaactcccttcgctttgtcgtttcccaacctgatgaaggaaggtgaaatacaacatatggaatgatatatatcacaaatacacacgactctggaccagtgcaaagtagttataaactcaaaacgcccccgacatacattaattctacttcgaaaaatatgttgccctaacgaaatggtttgcctaacagcggcaaaagatatgtcgactcgattgtatttaaatcgattattaagattgggatgagggccacgtagccgaaactgcaacataccgaaatgggcgttacaatgcattaattataatttattggcgctcagccttaattaacaatctaggcgtgctcatactgtgtactttaaagcaccatttacatgtcataacagattattgatgttacgtaaaattcatagtatacagtatcacctcgatcaaattcatatgtttttattttaaacaagagtactcctgtgtcgttctgaattactattagtcaggtgcgttaagctctgcagaacgataccgactatctgtgcatctacctgattcgaaaatgaaggcgattgggactctccactagttctgagttgtcctcctcgatttacaaaagataacttcagctggatgtttatcgaacgcacaaatcttaacaatggtttaagtagccgaatcagattcgccattcaaatctttgctctagtttcatcagtccgagttactctcaaaataacaacctaactcgtcttgcctacactggttctgggttttatatttagagacataatcacgaaacttcatgcactatagaaggcaccatgctgttc

SEQ ID NO: 298 - DNA, 2000 bp, Artificial Sequence
  Exemplary stuffer sequence
tgagcttcgctttttccagagtcgctgactaaagtgaagtgtctagtcgttgtccatgcgatatcggggtccatcaactagaattcatttacggtacgcgttgtcatgccttatatttagcaataagactaacggaagctcctctggagggaaagtaagaacgtccccccgggaacatacctaaaataaaggtgcatgaaccatcacggagtggagacgcaaaagatcaattagtacaaatcagcaggagacatgcaaagaccgcgcccctttctttttataccatcttaatagcctttactgatcgtgtatgttttcatcgtgcacctaattatggaaattctatgaagcttttgctcctaatcgtttagtaatgctctcggatgccacgttatcttactgagaagcccgtgaccaaagcatggtgacaatagaaccaatatatatgaaaataccgggttcgtctgaagactgtgtagtaacaaaggtattcttgtgaattcacgtttttaatctcatctactatcggatatgacaacaaactctgattagggtaatataaaatttaccgttcggcctaattaaaggacaaccggtatgtaaaacagcaacatcacctagcacgaaatttacctatgagtgtggaattcgttagcgctgtcgacgtgcataacctacgggttgttgcatacgggtcagtgggataatgttgactcggtccttagtaaagactagctcttcttattcttgcgcttgtaactgacaagtcgagttcacgtgggcgcagtaaagtcgggaagacggtaatcgcaaaagttcggtaaaactaacagtttttaacgagtccgtaagttcaagggcctaaatagctggaggattttaacgtctaaacattcgggacacagtgtatgacccgcataaaaggttcaaagaaataatacttagagccgtcgttcggatcttatatgtttgaatgaacccttaatcaccctataacatgaagctacgacacattaatcagatcaaaacctacttagagctcgtccgatactacaacttgaaatcttccaccaaaactaaagggtccattatgtcaaaataccatttctatttatattttaaccatcaattcgcctatacccctaatcagcattaatctcgcttaaagatggtagagttaaatacaacgcagagcttttatactaccagtgatggatcacaggattgcgtttcaaaaggtgatagcaattaccaatgacctttgacagtaatgttacatcctaaccggattatttggaataccctctatttgctttctgtttagccgacgcctgtaattgtctacctgcgtgcgttgtgatgccggtccgctcgatttaagcactccgatatctcatgtaggtgtggactttggacaaggggaaataactctcaatgacaatcgtactgcttatgttaggcaatgctggcatatgcaactctgaggctaactaagttagtcttgtccgtgatctcagaacagtaactatttagttgcttgcgagtatatttcggtagagacgtatcttctactaaacacggttaaatattttttggttatctctcgcccggtctagtagtgccataacgtttacgaggtcatataactgtcatacattgcaaggcgctttatctcaattgtgaacaagtaattatagccatgatacaatttttggacggaacttgttttatctaaatcgaaagaacctacattgcctcggcatagacctcggaagcagctagttcactagctgcttcatgatggtccaagcttgtgaaagattcacataaaatcaacctccgtgggagtctccgatggacgaagctgtgtgactggatattatctcatgattgcgtcacccttaacatgtgtgaggtagagctaactatagaaataccagtcgagttagcgacataatgcgaattgatccgcctgtcaattcctccttatacgcgccgtt

SEQ ID NO: 299 - DNA, 2000 bp, Artificial Sequence
  Exemplary stuffer sequence
attgtccattcttgtatttgaatcactccctaatgaaccaaactctctaagcccattcttgtagtatttaacacacatgacaacggtccaattttcatgtatagtcggagtaacgcgatatactgaatcttctgacttatcagacatataagatgtaaaaacagcggatcaaaagtgttctctgctgggtgaaaaatgacaattaagcgtggtattatctctgtaaataacacagggatttatatgtaaggatcgcgccctcatacattcattaattctcactcagacttccctccttcgggctacgttagattgaaatgaaaataacatgttgtaatcattaaatagtacatactgagtttttaaagtcgaatactacaaaaaatatcatactttttttaccagttcagtattggagtcgacacatgatctaacataacagaagacatagcgatggggattatcgacctttttatgggtagtaacaggtggttgccggatgcactagcatgatcaggtctcctactcacacagtccttctgactgttaggttgtctttgcttataaaaatactcggattattgcgccacaattatttgatcaacgagcttcttggagagaataaaaatattacacttcggatagataatacaggttaggttctcctatgaatttgaagatcccatgttcgttaccgtccaagagccacggcttgcttgctcgaaattaaagtgggcattcgcgcgggatgggaagtaccctcagtcttgacaattcccatcgtcaatattagaacggtggattcgccatcaccaggaaacgtattgctgatgatgatttcaatactgaagtcgtacacttctcacccggaaacgttaaaaggacgataatgacttaattgagatcatcgaggtacgagcccatgccttaggtcgcttcgtaggggtcctccttaaaggagactgtttcttacatgatttgttacttcgttgaaaataaatcatggatcgacgtcaccaattactggggtacctgagtatatagcgtagaacgtgaaagtgattacacctgtataggaaatgatgagctcggggaaccataatgaattatagtgtaaagataaaaaacttgccccgtgccacgagaaggaatgtagcagacaatcatggggacattgtaacttacccagactttaatttcgttttcactataccactcaattatgatgtgacattctggaattgatagcgtatgttgcagccttctaaactcaacactgagctccttaagggttattatggttatatttgagactataatataatccgagttcggtcgaagtgagtaatctttggagggtttaggggggcagaattcactataagcagcagagattttcttagaaagagccgggtcccgttccaataagccctaccggacgtttataatcattggtgcatcagtgaggccttctgttcatcttctattctgctgtacccttcttgcaccaacgcgttggatccttgtatcgagtcactgccaggtttgtggattttttgcagcccaccctacgttatatcttaacaatcggataattaaaccaagctatcgaatgctatgagctaccacagattatcatcgattgttttccctatcattacgatccctgacggactacttagtatgtccttttcttaatattcgttaagaactggagtacaggctgattacacaaccagtaggattaggattaaatagagaaatgtatccggaaaagcggagttactgtttgggtctttaaccgcgaatcgcggttttttttctaatatgcagtgatcctttatttggttactgtacatctgctgaacacgctatgtggatctcccacagttgcaagtgcaaaatattaataaattaatcacaatacagtacagctagatttcatactaaatgctgatttttgaccgcaccctcgagagtaattcaatgacggccatgta

SEQ ID NO: 300 - DNA, 2000 bp, Artificial Sequence
  Exemplary stuffer sequence
aatcagaatgagcagatgtaaaacatatttatgtaagcaggttatcccgtatggcactcgttgctctaagtagatgtttttgtctcgggtaacttatgtccccatcctcagagtgtatttacttttatttaacccgacggtgagaacatacaacgggtcaacaagacaatacgaccattatactgctaaactctcttcctcaggtgctatatgagttacgacacaatttttgatgttaaagtcgaccctagctgctaactgaacttctgggacttaaaactaccagaaaggatgaagaattagtttggtcaataactatatacgaaacgccctgaaggaagtcgtattaaatttggagtgcataagacatggtgagcgaaaactaacacctacctcttagatacagattacttttagttatcttctggtctatcgttgatcattctaagtttattcagcactagagacttttggaatacgactgccaaagctagtataggattatctaaagatcattattattaacggataatgcgaaatttgctagatcgtatatactattaatgcagcaacttaactaaagatatatttacagtggggcttatgcaaccggtgagccctcggttctttatgattcgtcaagtaaagttgcacaacgttcacgatttaatcttattctttgatcttgggctgatgtatcctcattatttatgatagaaaattgattggtgcatttgattcgcccgatactagacccacagctgttgttcgatcccgtatacaatgagagcatgttcagatcaacagtaggtgtaacatcttatgttccgagccttctagtaaccaacgaacacctggcaaatgaatttgccatctttccgctgtacgaataggggtaatgtgcccttgatttaaaatgttatcgataggggaactacagatactgagaactcctgaaacgacgttaacaaacctcctgcaaaacttgcactctttgaacgaggttgcctagtttccagaagtaggttcttgtcacttgaatttcgatggaattctccttatctatccagtgacgaggaagaagaaatgggtttttacaaggactaagtgtttagacagaaaaactaatctttcagtaaaggtgagaagtgattttgcagagggagattgtgttacgaggatagtactgacgtttatatgagaaatagttatcgataatgtgcgtgtctttaccaagggactgaccaactgatgtggaaatttaactcttcatgatcacataatttcaatacgttaacagttagaagcggtgatctttacaaagtagacaatgagttattgtcccatagcaatgcctaatgtcgagcgtgcttcaaacaattgaatggcgttattttttgatccttaggaaacaaaaaccagcaacgtaacttattcttgtatcttcatgtaatcacattaccggtatagagatggttttacatatacgcacgttactttgagatagcgaagcatacgaatatacacgatacaatgtcagaaggataaaatcactatggcctcactcggtgcatttgatttcaaaggcttaatgtagctctgttcgcactcgtggatatagttggagccagatagactaggaagatgtttgtttagatagtatcctcgttcgtgcataatatccttgagatagtataggtcgaatctccacagcagcaagattctccgtgagcattgccactctttcagtagtaagcctaagtaattcattaagcgtaattagagacttattttccatatctgcgcgtcgagtttcttctgcagccctagttaggagacatacgggacgcttgcgtttttatcgtagattcacttagtacagggaagataaacatgagaggaaatccgacacctaacaatactttcaaactgaggggctggattgtacttaccttcacatcatcgaagtcaattcttcaccttcacaagctctttcttcg

SEQ ID NO: 301 - DNA, 2000 bp, Artificial Sequence
  Exemplary stuffer sequence
atttacacccatgccgaacataaataaacaaacacaaaaggatgagaggaataatgggttaactaaggggagtcgaatcgtattgatacttatgaatggctatgttacactcaggttgtactggatttcgtttgcgctacagcttagacctttcgctaaagatacacgccgcagtgtctgaaacagacgcacatttaaaccgctgggctgttaacgctcattctcgctgaactagtctgtcatttatcagtgacatcagcttatctccaatcctcataagaccgtcgacaggaaccctcaattccactcgtaacagtcccacgctgggttgcgtagtctgttgtaagaattcattcatggttgaaatggggctgatgactatgaggcggcatctattggtatggtttagtagacgatcagaggaagtctgtatagtcagggctcaatatgtatccacgtagtaatgttgcctgctaccgacacgatttagacaacgtcagcgtaattacgaacacgacctcggttccacgtgtcatcgtctagatggtccctttgttcgtaggcctccaagacctcagtaatatctaattcgagcttcaagtttgctagacgttgacttgacgtagcagataaatcgcactgtaatggaatgatacctgaatcccgttaacttccagcatggcacatacgatttttaaattacgcttaagataaagaagcagtgcggtctaatccaaagtgcacaagcatatcaaaactcaggtctggtttgtacgattatttggagcagattttcaagatagttatgccaatctctccataaccatatacagtgacggggaccctctatgatacgtcatctccgggacctactttgacgctggagtcttacagatggtgggaccatttgtgcttaagctacttttagtgcggtaggagccctccacaatatgattcaaacctaaagaagctaggagccctctcgaccctggtacttggcattggcttaaatttcacgtatacgccatagcagattagtttaatctccgattttcaaaatactagatagggagagttctataccacattaactcgccccgatgggagaacgcacaagagttagttttcgacgccgcgtaaaacaattcaacatggccctcgagtctgctactgtagtgcatgaaagctttcctagttgggctagtagcccaagattctggaaaaattcaagttagtcgacagatgtttccgccttacgagtaatttaaagaggttaccccgagaccgcaaagagtttagtgcatcttatgtgcattgtgttgttcgtcagggggctttgcacctaaacggtcttacgtacaagctcagttcgtggatacatgaaagtcttggagtcaagacctacaaatcgacgcgattctaagtctaatgtatccttacttcgggcgtattgtgatagtatcataacggttaagacagtttaggataaaccgcagagacaaaaaatctcgttcgtgtaactgagtatatagtgtacacttgtgcccgcaaatgcatattattgatcgagtaatttaacgtgtgcctccttggtagagggtttccctaacatactccttttcctgattacctcagtctcctgcttcaaccggtctccataagtgagaggttgtgtgtaccgcactttagaagagtagaggtttggcaaattttgggagcattagactagtcgaatttcatacttcttagtcgtctgggagaacgtaagacctgattaaacgcatgatacacgaagtcattcagttcttcagttaagaggttgcatcaaatagcactagcttaaatgtaaatcgtcttaagtccaactattatgcggcacttgatcaccatttcactcacctcatcactacgcttgatagtatgatctcatcgtgatggtacccagttgagatcagcgaggatctcctcataaatttacacattgttaaaaggtcccgcgc

SEQ ID NO: 302 - DNA, 2000 bp, Artificial Sequence
  Exemplary stuffer sequence
tagatctgctttgtgaatgccgaatttcagattgactgtccgcgcgctagctcattatgacccggcagttgaaatcgtatagggttggacccaactactaacggaactcaaccactcgccctgtacgagatcacagggaacgtcggctaaggaggttatggtggccttaccttagcactatataaagtgcgttcgaaacctcagtgattccccgatagtatgatttttaagttctaagattaaatttgatacatcagttggtcctagagttagtgctactaagcttaaatcaaccaaaattttacccgttctattcagaaggaaactatagtggtagcaagtgtgacagtaggtatagacttaaatagttacggcgaaatagaaagattacgacgttcagccttgtgtatcgaatttgtgactttagaggcacacagagtaatggacctatcatctacgtcctgtcagagtatcatgtgcatgattcgacagaaatctcaataataacccaaatcgggctctcttgcattgaataattcatcatcaacatgaggtaatagcaaaatgcctttacttcagttgattagggtgatggccgatcacctatgtatttgaacatatattgtatatccggtcggaatatggcatccttagccgtcgtgcgccggctttcggaatttgatctgtctctgtttagacgcgtaacctcaattcgccgcaaactagatcactattctaataatctcactaggaatctattcgacatgcgatctttgattataggattcagaatctaagaaattgctacgatggggtgtcatagcgatgtctatttgagtttctatagtgaattggccatttgttttggcatcatagatcgctgacacaatcattgtgtctttcatcgatctggagtacagttagaagagaagcgagggctggtaacatgcttatagattcttatacttactaccttagggtacactaacaatatttgacattataggtcgaccaaaaagatttctctatcaggtttagagacaaagtcgtcgacatatttctgtttgaactcttgaggatgcacgaaagtgtctatcggggtatcagtgagaaggcgtggcaagcattctctaggtgaattccaccctttttagtcctcgttagtaccccgtagaccgcggaacatcgagaagttattcgtaaacgtgtctatctgttctatgttaggagtaggtcattgaacaaattgagctttcaaatagattctagaatgtagcgcgtaagtatgtcccgatagcggttttcagtgtattagttgcatctaatgtaattgagatgaagaaaaccttggtcgaagagacatgcctaaagaagaaggctaagtgaaggcctttatatcacgtggttcatagcccattatataaaaatttatattggagatgtcccattggtattgatagatggttggtagctgtcagcagtgcgccctaggtaaaccagaagactccttaacagatcggtataattattcgaggtttccggctctagcattcagacatggaaggttctttctaagcggatatattgctcgaagcccgtgaacctttagaatcaacctttattatctctaaccatcttttttacgtttcacctttaacttacgcgaatcgattcacgactgccgaagtacaaacgatgactcagtgttggttttcgctacaacattgagctcagctctatagcgcggactacaagttctgcgtagattttgccaaaaaaagttgcgggtagccttattcatttaacgtatgactgggaggcgctcaaatctctcactgcacctattcgcagacgcaaattatggcgtcgaccccaaactttcaggtaaatagctcacaagattgaccattggcaagtttgaactagtgtcgtaacgtcctgaacaaatgtttttctagccgctcctgctaaccttatggacattttcctcttcacccctg

SEQ ID NO: 303 - DNA, 2000 bp, Artificial Sequence
  Exemplary stuffer sequence
aaactacagaagaacccaaaggctactcactccctttgctgtgttcagctcgctggctcgtcaagataacggactcatgtctgtgggcaaagcaatttattacagctatacctttgtggaaaagtctccttgtaaaattgttagcaatattgtttcgagttatatcgaatttaaggtttattgttattcgtgaccataaggagctaacatgatgcggtttaatgcgtatggaaaagcgatagtgtttttagtgagggaatgtagaagacctcgtttcaacccttaccatacccgagggtgtcttaatctgttattaaataaagagcagcaaaataaaaaaaaaatgcagtgtctatcaaattcccaaatttggctacgtcgttcactaccaattttcaaaataataagaagaagtatatggatccagtctgattgtctttccgatcagcaatataaagcaccaacgtcttataagagctaaatagtgatgattccatgcagtataattcaattcccctaaagctactgtcgataaacttcatataacatatgtacttggaccgtttggtttggacttgacaggctttaagcagtctgcatcatgagcctccttctagatgtgcaagcattccccagaggcggttcgcttcagcgtggtaaggaatgatctctgggtcggaggtagtgcagaatgaccacttatcctatctagtggtttactttatctaaaacaacaggggactagatcttattatacggccaaaactgaaatgaagatcatctcatgaatattctcttaacatgagaaatttccgttgtcaatttttaaatggattaatgtcataaaatctgggatatggcgagcttaacacaatgcccctagtttacgttaagaaacatttgatacatcaacaaaacgtaggatccgccccggttttttggaatccacttctagaagcaggagcgggtcgctgtatttaagtcataaaggacgtcgttttacgaacaagaccgtgtatgaatctggactgttacaacggcccatccccaccactagttatactagtcaccgaataatctgaactattttactagaaagtctagaaattcatcctttgacataaatggattggaattaaaaaaagaatttcaaatataatcatataaaagtggatgcaccagagctcatgcgacgtcattctacgagcgatttatagcttataccaataaaccccgcgtgtattaacggtccagtcaaaaatactatgataccgaacaaggtttatcgacttgtcccgttgaaatcctagatgaagtttataaccaaatggcgcccctttagtgacgctgtaaacgcagatttatcaaacaggaaacatttctgattaaccagaagtatgcgtagtgaaggtatatcgcgcagtaacattcaggtgcttcggggattcaaaaacgtgttgctggtatagctcgcctgttttatcgaatgtagtctcaaaatctagccgagtttatcaactggtcgacgctggaagtctgcacttgaacatcgttcacatgtaagccagagataatggcctcagcatcgtcttattgctaatctcacgctgctttgtcgcgacgtactctctgcattaccaaatgggattagtttaatttcgttctctgggtgaccttgtgcacgctatgtgggtttgtattagttgattaaagagtccctttgaagatggcttcactcaccacatgactacacttcctatcgaggtaaggaaacgttttcttgtgcaaacaccccagacttaccaagtttaaagttttgtataatattaagaatttatctaacactgagacaccatacacagcttccgtaccctattggtccacaatataagacgttagatattgccaataaatgcttcattcggttttttgttagacaattggaaaatcttatacataacatataaacgtttcgcatccctggttccttccgataggtc

SEQ ID NO: 304 - DNA, 2000 bp, Artificial Sequence
  Exemplary stuffer sequence
tcgttttatcacgttttaacattgaatctttagtgcaaccaagagccacttctcctgggttataatcatcatctatttagcataccaacgcgtttggctgcctcggtttgtatatagtcgtaaaagcctccggtttatgaggtgatggaaattagttggatacttgaatagataatatcccatgcggtattcacccactgaatcacatcgcctgatgatccttgctgtttgcgggagagctcttctaatgatttttgcaaatgctgtgcatccctaatagtcttttacagggcaaagtacagggattgacagcccccgaatgtctacagccgacaaaccgaaagtcttctaccccgaggtagctgaaggtgcatagacgtagacatgttgactaatctcatcttgtctactatcttgtacacaaaatcaaaattacaattatatggaaggcatgggatgagtgatcgttaattagacaggggcgtctttggcaatgcattctcttatgataaaaggttgaccagattactgctcatgacttagtgtccaccggcccaacaattaataattaagagactcaaccgacatacgttaatacccaataatgccccaatacccagacttttacagggttattcgtgaacatgagtccctcgacatcttcccagattttaatccccatattactagtttgtaacagattggttatgggactgattagaacagggaatttcagctggaaatcactactaacttattgctagtttgccgatctaagaagagtctttgctaattgattttaaagagatattctgaacacgtcaatatccaaattttatccgcaccattctgacgtaatgacgcctagagaacgagttggtggcagtctatcgcttctgtttattttaaccttcaaaatatgataaggccccagttataaactattttttacggcaacttcggattaagtgttctatacgccaaaactattgatttacttaacatttcatcccgagaagctccgtcttatcaagtacgagatgatcccctattagaaaaaccacggctagtatcaacgacatgcgttacacacacgcctcagtgggggccgtcacacatagttcaaatattgatactgctcgtctcgatatgtgttcaatgtcggcaatcaagcagtgtcggaactgaacccgcactacgggctcgtaaacgacccaaaatcccctaatcaatcattgtagtaatggtagcaacttgtatgtcctgtcaacgcaacaccctcctggtgaattattctattagaactactaaaaaataaacccgaggtccagctctatcgtacacgacacgaaaacgtatcaaggtacagttcgatagccgtacttattatggtgactagcgccatatacaaggtcataagggaccttgttagcggtgtgttcacttcatcgtcagcgactcgttcgactgtcatttcaatgaaatctttaatgagtttaatagagtaggaagggacagtaagatattttatgaataatgtcgtacgtaggatttttttcaaatgatgactatcacagtacggcatacggaaaattcagtagggaattagatcaagtgtaaaattactggtatactagcgtatacctagtacgatgataattaacaatcacccccagcatgatgtgagaatagtaaagtatccatatttacaactaaaaagctcggaagctgaaatcccaaaccgcttgaacagctctcgaataataccggtgtttatcatcggaaggacagcgcctcaggattttcggcaaatcatagctcttatcttcgatctaagcgtttgatgaatattagaatcggactgagatataaagaatagtgatatatgtcggaaaacgacgatgtcattttagactatgatcttaagacggagaaagctaccatcataacaccgacttgtcctgccattgtattactggctttccatcgtgagggatagc

SEQ ID NO: 305 - DNA, 2000 bp, Artificial Sequence
  Exemplary stuffer sequence
attatgatcccaggcttcgttgagtctaatagctatccgactaatcaacttctcaggcatgtctcgactccgatcctggtggccttaaatttcttaggtgcacggaattgtgtgtacctggtatgtagagactataacgactcacttcttgccaattaggattcaaaactccctacttgagcaacgtgttcccccgcattatccatatcacaacagttgaatttttctaacgtcttctcctcaaaccggagggaagtgtgaatgtactgttgtccggccatgcctgaggtattttgattctagttagtaattacattaggaactcacttcgtcaactcaaacacgttgacaaatgtgcagttgggtaatacatgccgtgcaaagcatgtatgaccgtggtctactagatggcttcgcgatttactgttttgcgatataggcgtcggaataaacttcagcaggtgcggatgctgatctggcgccgtcatttataaagatatggctacgacttagctcgtgagatcgagacaaaatcaagatcttatcgtcttccacaaaaagtaccctcaatcggatattcggaccgtaaaaaagagcatggcgcttgattatcgtagctagcgcccaaggaacaattgtattattcagattaaaccccggattggacctattttcatcctagtagaaacggtgacgacgcgacttccgaaaactccaggaacagtgcggtctacccaggttgtagtagatgcccgttttctcagggcaaccagggcatcatacgttaacttaatcggttttaaccgcgaagttcgatacggactgatttaataataaacgcgaacaacctagtaatatcataaattgcggcgtgtacttcagaaatggtaactaaatgtcagacttcttgaaaaggaacaagcgcgctttctcaagtttgttgagtctcatcataatgggggaactccgtacatggtccgatggactcgatatccgaaggcgataataattatccccgtgttctacgctatttacgaactattaataatgatcggtcatgtcggtggtttattccattcctttatctccgataagtacgttaccatgggattacgcaacagctagattttcaaatgatcgggtcgaatccggcctaaacgaaacgtcgctagcgattgagaacggatgtacagatctctcgaatacatgagatgcgcgtaatcatagtgtacgatagaacctcatgttatcaacaggtgctatcttagtaaaatacatagtcatattctttacacgcgtaaagattctttgagccagcgaacatggaaatgggcgttggtgtgtttctccccggctttcgtaatagtcgccaccatccgcttgggtgctgattcgatcagttctaaccaaggagcctgacagtcttcgatttttgtgtattcctgtagaatatggcaccataattcagcgggaaaaaattgtcaactcagcagtgtctattaagagattactctcgcttttggactggtacagcctttacctagtaatatagacggacaaaaattttgtgagtcagacggcatatcctgaaaacaaatacaagtgtagtctacgttttagaatagactgagtggcgtcggtagaagttactgctcgagttattgtaaaattcttgccaagaacgaagttactccatatggaaaagatgactcaatcgagtcttactagattatttccgaagtcttaaacgtttagacctaacttagtcgaaagttgagctccagaagtcatctctcccagtttatcaatagtgggtggaacaaattcatcggctgttgaccttattgcatccacctcgttggagttatcttgccatgtatcctcaagtgttccgacctggaagtatgtagaaacccctttgaaatatctatcacaaagcaatatcttatattatcttcgtagtttttagaattatatctatttaagggcacaaagtctag

SEQ ID NO: 306 - DNA, 2000 bp, Artificial Sequence
  Exemplary stuffer sequence
ttaacaataaatgattaggttgtgcttgcctcctaattttgtttaaaaagttgttcttctgctgactagtttgattctactcatttctgtagtaccggttcggcgtactttttttagaggaaaatactaatgtgcggaggagggcttaagaaaactgcagatcactggatgagcaggaaaaccgaaggacgtgcacgaaaatcggacttgctgttgtgactatacgcaggctagaatcaataccgtcggtgctcgtgcctcagccgtatcagatatgattcttgagcgatgttatcgttggatcaaatagttcttttcgtggaaaggtatggttagatatccggggcctcttaatattggtttcgactagatctgacagagtcgggtcaaagctaacgctgtcgctaatgatgacagtgtcaatctggttaagtatactctggagttattagtcgatctctctcagtgtttcttaaggtgttctcagctggccgggttgtgcgcttgtgagggagcgatagcagtttgtgctcggtctacgcagtagatcgttcacaacttagtcagaccaatttatattcctatgcctaagaaatagtagatcatctaaatgtagttgccgatcaactcaaaaatcatgagcagtgataaacgctagtacggagctagcatatgcgcctgccgatagattgcatagaaccacagaatctctaaatttctggcactgactttaccttacttgtctactgatcatttagttctaaggcgggtcccagcatatactgagtaaaggaaattgcaacggtccaacaaagaatcaataagtaaatagaactcatcaatctccatggttttttaccctgtggtatgagagcttcgagacagtacaaatacattctacgagtgcatttattaaacacacggaccctatacaaattaatagcatcactagctcgaaacctattacagcctgaacgtttcgaacgcacttcggtatacagtgtactcgcgcgcgtgttgaaccgaaggtgctagccgaattagttggattcgtatatatgtgggatcccgatttccaagtccttgctggtttaacacacggatattagttgctattattagcgtgtttgaaaaccatgtcagagttaacgaccggctaaaaagccgacttataaaaagccgagtggtttggcaaccttctactggtcttggaattaacttctgaataaatacaaacatgaaaagagtgaactgctagactgcacctgtggaatgatccataacagttaaattactccgccgagtccattttgctgacggtggattatcctaactgaagagcgtacagcgattctgtccaaccgttgaaatcagtaattttctatacctactatcgtttgaccaaactcagggaagcatacctaaatatcatcaaggcgagaaacttttagacccatagttgtattatagtctaatttcaatgcacattctgttcaggcacagactgatattgaaagaggcccgcgactttgaaggtgggctaaatttatgcaataatggcacaccaatcaacacagtctagaacttaccaaaccaagcctagattcacctatctatttttgatccgactgtataacgtattgtaatacctcaagacataagacactcataacaatttaactttctcttattaggaggctcctctatgggattcgtcgtcgagttaaatgatttgaggttttatgtggactccgagcacgcccggtaagaatttctaggacttaggatacaatgcaactcagtggagtatgttcccccgtgtgatctatatgatagctgagtacgacaataggcatgcgattcagactatccgcttttaattaccaatgaatgtcacgacggagaacgttatgaaaggttttctctagcacgccctatcgctcttatatgcgaaatacattcctgcttgtgaatggccgggattgcttacacattagcct

SEQ ID NO: 307 - DNA, 2000 bp, Artificial Sequence
  Exemplary stuffer sequence
cttaagatttcagctagaatggttctggcgcgcctaagaaactaggttaagtcttcttttgcgcgttaaataaaaattttgtcggtagttcttaaatggtgcacgaagttgactgcatatatatatgaagcacctaagagctctatccccccttaaatgtcaagattggctaatataccaccccatacacatgattaacccggttaccttcgacaggtttggatctttaaatacaattagttgatcttcgctctggcagagctcgggttcgttcgtagtgtataaaatatctctacttgcaattatcgtttaacccctgcaagagcgtctattggtcttgctgttttcttacagttgtatgctcgccatgtataggcaggtaaacagactttgacaagggtgggcgagtcgcgtagaacctttccatgaaggcatttatttttgattatctctgatacctgggtgtgtataattggatgcaacgtcgcttgctaagacattcgagctcgaaattctaggattttgtctataccctttagaatcttcacttctataaatgactaaaaacatgggaaatgacaaattagcaagcggcgcttttttgaatcaatcactagatatatttctaaaacttagcaatgctttcatgaaaaccactaattttaattacatatttgtaaataacccgcatcaaacgcaagttgatgtcgcatcatatatatctccatagtcatttctattcaactggcatgttcggttaatcaaacaaacctgacaacattattggtctcatcaaaatttgctctattggcatccagaagattgaattttgagtgaccagtaatattaccctctgggactacttgtatcttttgtaaaagacgtataattgtagggaaaatttgaagttgtaaactagaacaatgaaataaatcacaagcctcttaaatttccgagtgtgtttaatagctgtccgaagaataaatatccagggaggatctgatctctaaaaaggaaactttcctaggtgcaattcatgggacaatagtctttaccatcatttggatcggaatctttaaagatttaacgtaaaactgtagatgggtgaagcaaccactggtgtcaggattgttgtaataacctacaatacgaaaacacatggaaatatttttttcacgagctatacacgtagttatacgtatgaaaacaaacaggactcaaataatctatagaggaatttataggttcttcgtgaacgtttcgagagcatagacatgattacaggctgcagatgattgctctagggacactggatacgtctgtctcagtatattaagaggcattaacttatagagctggtttgagttcctcatgagagagaatatatatttgcacaatgatactcaaaaacttaccgctctgcacaatccgcacatcgcgatcatacgcgccgttaaagttatcatccaatatactcataaatggtgtaacctagctcctaccacaaactgagtaccgggatcgctatccacatcgctgaaacaatgggaaaagaaaggtttccttcgagtcacgcactgactagatctacaatacttatgctctagaacgcgtgatatttctatgtaaagtaaagcatgctactaaggtacatctaattttacgaaaccgtatactactactcgccattggtatactttagactttgtaagtaaaaaacgagtagggcctcaaggacatagtcactgcttatacagcgaaacgaagctgctaacaaagctcagaccggtattgctgttagtatattcttgttagaagcgtacatcggttgggccgtatggtccgattaccttaagaatagttgactaggatcgtctctaaggtcgtacttacccacctagcagctgatatcttcgatgcctatatctgtataggtagagattcattctcagcgcattgccgcggtagatcctatgtagattatttagcatagttaatta

SEQ ID NO: 308 - DNA, 2000 bp, Artificial Sequence
  Exemplary stuffer sequence
gaaccttgggtccttatcctgaaataaaaagaaagtgcacgtctccgtaatatatggatgtctcagtgatatccacgattacatcaagctgagttatttttaatgatagttgactgtattgcctaaaacgtatctgtagtaatgaatacataaaggtactggtgattgagaagttctcattaaacgttaaaatccgcatcatctgtaaaaggtgggtaattgcactatagagggtagaccacgcctgtagcccgcttagaacaattcttgtactatcatttttaagtccttcaatgtctatcataagtattggacattgcacgagaaaacacgggacaaaatgctcgtcgtttgagactatggatcgctattcgggtcgagcaatctgaaacagatattgtcatgtttggaaggtgagcccattagtagtaagcgctttataccactattcaggagtaataatttaaggagtgtaacagtatgatgtctaccggtacacgggagattgtaatacagtagtagctccttatggcttgggaataaattacaaactgaacgctttctttagagctctagtgtcctgatttatgggtaaggcgtattatctgcaagtctcagttcgggataggtattccgtcatctaatattacctctagggtgtatactaccatcctttgcagactataaatactatctatcgtcggcactgatagatggaggattccttgcaagacctgatatctccgtctccatgtctagtttatagatttgccttacaagttcatttatgcatgtgtaatagaatgatttatatgaaccgtcatagttccattttagcatccgagcgtgtgtcctctctcgtaattaggcgtacgtcgaatcattttgctttcactgtaaataggcaaagcaaaatgtagcaaaggaaggaatgaaatgatcattctcatgctacatgtgtccttatacataaaaatatatatacttgattaattgcacatgaatcacttacattcgattatcataatacatcccccactcggattgctccacgaccagatggttaaaaagttgaatctgtgctttgatttttaagtgagcactcacgtagtatgaaaccgctagctcaggttttttttggggatcgttcagtattcacgaaagaagaatgcggcggggtggttccacaccatatcaactagtgtttatagttgcttatataacggcaaccggctagtaaatggtaacttaacagtaaaatgtctaggattagtaaacatatattatggaggcgttaaggctgtacgccttgatagtacacacctttttacaatcacaatcctaggttgatctaaaaccgttgacgtcaagtccattataaaatcttaatcgcctgatttccctgtcctaaaatgaagagattaaagaagtgaaatatatccctaagccagaagtgggagaataccatttggatatatgcgagcttctgccaaatcttagagatttctggacttttcaattatccaatatgaggcttgaggattaccaactctggactacatgacagttccacagaaactatttagttagacgcagagccaattagaacctcgacaattaggtaaagtaaagtttacaatactgttaagtcgcgtaaaaaaggttgattcaactatgacgggtatagaggaggaaatagaggctctcgttagctgtgtcgttggacatagtaactttttacaaagaatgttagagctgttgaatatttacgcttatacaaagtatctgctgtatcacgacggattttatccatgcagggcagtaatccatcaggcttttggagaggacagccttgggaaggatatcgtcacgaggcgtttcgcactcagacacccgaaaaaattacgaggaaatgataatcgtaacgtggcgcctagcgctggataattaccataatttaacagaggccacaacaggttttcacccttcaatgagtgtaa

SEQ ID NO: 309 - DNA, 2000 bp, Artificial Sequence
  Exemplary stuffer sequence
gattctgtacaattgtttcaaaatatagcttaacacatttgatggaataataagggttccaactagatatagttagttaggagttacgggagtggtgctcgggtacaccgaagcgtttatgtctaagctctcttctgagggggctcagacagctggtacaataattcatccgagccgcggtgaatgcggcatcaggccccttctatacttataaaagagcatatctaatttattggcatattcctgcaggctacataaagtcactcggtcgaggcatccctattcgggctaaatttcaacacgtctggtttgaatagcgactgttttttacagatggcttggataaccaatcaaccttcaagaagcacagttcttatgttaggaaccgtatgcaaccgtagactcctattttcacttgcgtgagcattcaacgaaattgggaagacagatggacttacattaacgtatcggactacgatcgtaatatccgtgatgtgagtattatagtatacaagagtgaggagatggaaatcatgacggttatcccacgtagcagcacacgcagatgcagaccagacagatacgaataaacttttttgtacggttgcccggtaaactagcctgggatcccgcgaacaaatgttagaataaaaacgcgagagacttgctttagtagcttttcatcaggattccttgcaaaaagttaacacaaagtaagcgtgttgttagtaatgtaatgtttgtgaggtaacactgtgggttaagtagtactaatgatctttctttgctgtttgactttcaaaatgcgtggagttcagtggtggcaaagattgtttaagtcttacgtattggtagtactcgttaagcttgaaagtttcgattatctctttttattccgatctgaaatgagcttgttctatccgaagctgaggtagtccacttagaccgatctatcgctaacgagaataatacttattatttaaatcctttctcatgccaatagaggagactgtcatggtaaccggtatgcttgtgttcatattaattctaagatttgctacaggattaagtctagttcaagtcctattccaaataccacaatctctaaggcctcacacgccttaacagaaaggggattatacgcgtcggttgttcgttatgccttatagtactcaacccataaatagatcgcacataagagtatgaatcggttgatgaaaaagtacataactcactacagtgccggatgagagattcccgtgaattaactagtggctacaaaacgtaacgtgcgaagagcaaaggtggccgcgatattacctttactttcggtgccttagtaaaagaggataatggcaaaatgaacgtcctgggcaatcagaccagagggaatatgcttagctattggctttgtaattgttgtagtttttaatggttctaaatatcaacaaataccatcatgatagttaccgatcagatgagcttgagccgttgaaaagaatgcaaatacaaaatcttgttcattaatccgatgcaacgtgccggcttgaaattcattttcgaagtagtgcgtccccgcgtatagacgctacagtagctccgaaggtctattgttagaacaacattttagaaacgggcctaataggagttcctcgggaaaaagaggaagggacaagttgattgtctattaagatagatgatcctattatagcgatgtcaatactacgcccagtgacaccatcaaaatagactggaaatgatggtacgattggatgagaagatcattagctgcctttaccttcgacgacttcgtcgtagtgagggttctgaccaatgtccatagcagttgaaagcgcgacattactcgaacaacgctgtggtcactctttaatgattcgtataatgaatcttcctctgcaacagttggacagaaaagtggcttcttgcttaggacctagctagactttgttgcctttctatgtaatacgtacgcaaattcc

SEQ ID NO: 310 - DNA, 2000 bp, Artificial Sequence
  Exemplary stuffer sequence
cagtagatgaggataagcccaagtatcgattccaggaagccgccatatggagatatagaggtatctctggcttcgcgaactcacaaaggagtgtctcgatggacctccataggtaacaaagatcaaggccccttaccaactcatgttctataaactgacatctatgcaataaagttaacaccagaaggtgggtcagaccacaaaccacaaccccgctcaattttagaacaaagtctactaagaggtgcgaatcaagccgaaaacgggagtttattgtccatatgatgctggatcggattattgtattataatagcctaagatcgtgtctccgatccaaatgcgtgtacgcatcaatcctgagagatccgggatggttgctggggttaataacttctcctttatatccggatgactgctaattcctcaaatgcaatcattctggaattatgaggcctattaaacgaatttaacagtacctagtcggtagaaacaattctaccccgcatccttaagtctactttcagagctactggcgcctttgacgcataggtaaaaccggcgactagaggaatgtcgtatcaagataagccctaatttacttatgctagcctgtgttcgataaataagatgtctgaattgaattcgcgcagaaaccagtgctgccacggtgaagagtgatcggggcggctatcaactacgcggtgaactaccccaaaacatttaggacatgcgaatatatcaaagagaaatcaattccattagttcgaagatgagcacgatcgttactaactgcagacaaagaaggcactattgatagaaccgattgacaacccgaacgtgtaccggagtttggatcagatcttgagactgcgcttaaaagcaagaacccatcacaaaaaggcaatagcattaggaggaatcgcgcacaagtacaataactttttccgtattttaataatattaattgtccttctcaccacgaggccgtttccttcgtggaaccagtcgtcctactttctctccgtaatttcattttatttagaataaaggtatatacggacgactatcgttcggaacaactaataacagtgcttggaggtgaatagaagtaagttgaactgagctaaagtgaacaactacaattcgtagccctgatttcattgtcattttttttctgactcaacaccccaaagatcgcgcaaagaataaggccatagctcaaacccgaaaaaatcttctaaggcctgataacttagttattatatgaacaccggtaatccctgcatgcagcatatatgaaataaaatgccgtcgttttcattgtttcgtataagtagggaacgaggtccatgtgctattttgctcttttatgtgtgcccaaggggtactggaatgtcgagtaatactcagtccttcaatgctcatcttgtgaccaaattcattggggaactccattgggaaaggaatctgtgagagtgaatccagactaggatctacccacattgtagtctgaattttaccttctagaaagtaccgctcaagttgactatattttacacaatgtgggctgatggctggtctccggttgaggaaggatcaatcatactcatcatgcatacatgaagatatactagtatgattaacaataggttttcaaaacagacactcgacttattgagcaccctattggctaagcaactgcatctgcactagcaatggatcttaaggcatcatataaccggttaggtactttcttgttaggtagaacaacacggttgatcaggccaatcgctactgaagtaatgaaatcaataaacactgagtcttatgaagtactattacaatctcctagggtcgtatcagacctttgttatgttttaaggacaatgcgggatctctcatccaaaaagcgaaattgataccaggcattggtagtcaagattaccgaattattttacgtaggtcattatatgcctgcaattttggcgctttacgctca

SEQ ID NO: 311 - DNA, 2000 bp, Artificial Sequence
  Exemplary stuffer sequence
gtttaatctccttgactaacaggagtctcttgccaacggatgtacgtaaccgtatgttaagacattatgaagagttaatattacatgcaaccattcgatttgccataaatgtaccgaacgccgttatatttacttactggatgaaagattcaagaatcaatataagttaaaatcttaaaaagatcaatcatacgtataaagtctatttgctattagagacgactgtctgatttgatgatgcagcgcgttgttataaacctcataaataagaggcggtggctttcttactattagcacaagtctcactgagtagtagaataactcttactctatatgtttcatcaggtacgaccccacgtggcaaaattacattttgcacacgaggcacattaagaccgaagagaacatttggccgagaggtatgtcaaagccggcttaatgatatcgacacaactcataaatggtgaaagttataaccaggtaatcttatgggattctgtggagtaaagcccattggacttcggaataaataagcaagctaatcagttataatagcatatatgttaataccaagcgtggaatgagcacattttggcagtttaacactaagcttgataaaactcgtagagtagcgattggacactacaagacgcgtgtttcgctagagacgaaccaccttgtgccaacagattactctgaagctcgcctatttgtggaagtaaatattacgtaacggttatagcattgttaacgatgattttgtcgagtaacggtatgaatttatgaaaaacgtcaaacaagcgtgatcagtttcgcatgatcgaattgagtttttgcccgcgcagggttcgcgtcaaaacaccttagagtaaatacttaagaggaatcgctacgtctatttgtaaaagtccgagtacccaccttggaatccccatttttttttttccagtcagctcaacggttgaatccacgtgtccgaagaagctctgagcaaactatggtgtcgccgttctaagcccatttcaaacgttatggagcgttgtgcctctttgttggcacttgttattcaccgcggcgaagtaacgcgctcgtcaagcgaatcattttatgcctactcgggctatagttaacggagttaaaatgcttcaagtgtaggtcgacaaaagatcaggaattcgagataaactctccatgtgaaatagcaagtttacgtcctcgtttttgattatagactaagattacgaattctttagcgctggctcatttgaatccaaaaccgtagaataagaaccccagacttatgtcctcgaaattatcaggtaagagaacaaataattcacgagtactgacagtataagcgcttatgtgagacgaccacgtaactacaatttataaacttgaccgttattatgtagtatttagtggctcataaaaccagcttagcttagatctgtgagactgaccagctgacccacaagacttttacattgaagttgcagctatatggaaacgtactttataatttcttaatgtaagaataaatttgctgtatcgctttgttcgtttgaactcttttctatgtaaaaggctgactaacccaggaagaggggagcatattttacaaattagtaagcgctctctcattcatttaatgatcaccttataccgacttcagcctatggaagatcttgcgctgttgcgtacctacagcgggtaaacggatgtgttaaacacgatagtaatagtaagtttccgttaggctgtagtttataacagtaacataagtgctaacgagatcaacacaattcaagttgcgaaagcaagaaaatcttgctacatatatcttagataagtatgaaaacatagattgcgtttttacaaaaagtacgaaaacattatattctcaagctcacgctccatgaacatgccatggatgcgagagctacttaatattatccggtaattattaaagtaactaccggttgcgcacaacggcttaa

SEQ ID NO: 312 - DNA, 2000 bp, Artificial Sequence
  Exemplary stuffer sequence
gactcttcttctcagtccacgtttgaaaatcagacaactacatattcaatggaagcgctgagtcggagtggctttccgattgactgcaggtgtctggcgatagattattaaaataaccgaggacctcatctgtgattacttatgttaacacgtcgttacaagcaaaatgtacagatcgtgtgtgggttaggggttcactagaatcggtggggcaaatttgccgcaaccgatatcgtatctgtcgccatttagtgggagctgggcgtgctatcagaatttatttaaacggtttggggacaaaagaggaccttatactggtagtataccttctttagtctttgctccgattgaatacaccggaacctaatttgtaaagaggcccagatgttggacagagtggttatgagtgcaggtttatagttcaagcatcagaatagtattaagataaaactgagggctttcaggccttgatttaaatgtgagagtattgtcaggccatttggaaatatcataaaatcctttgtgccagatagttatgaagctgcttagatccacttgccttcatttgagtctgctgactgccaattagagtcctcctcggtacgtatgaatagaaaacttcaaatacgattctccccaatttgctctgtgcagccttgccgatagtcctttatgtcatacactaggtgtgagctccaagggtcttggttccagccccgcaattcagataaacataagccccagtagcggaggagattttgaataccaaactaactttataacccgcgcatggccagtgccatagcgaatgcgcggggagaagtcattttagaagcctatcaggcgatcccggatcattaccctcgtataataaatagccttagctgcaagttcgtgtcgccgccaacgtattcggtatcagactctgatgtcctttaatagtgattatgacgactgtcataaactttgtagtagtgtatattatcgattgcgttttattcatcttgatgatgggatacatctgcacttttgagctaatctaagatcaaatatctattttcacgatcccgctactacggctcgagaaagttactttaccggaccgggcttaacacaagacttacgacgtcctggatagaattttaggggtttctaaattgatccggtttgagaacttcttacttatattccagtttcgaggactaggcatttcttcattaagaccgaggcatgggttatttttatattgtgatgcaaatcggtttgccccgccggagagactacatgccagttggtaacgtgacaaggcatgtgcaacgttctttagtgtcgctacgggattctgaagtctactgcttacctgattataccacggttcaacttcggttacaaaggatattcgctattgcacgggatggaaatttattcatgtcccaaaaaacaaactcgacaaaggtgcccacatgcggcctcattttacagtgcacttatgagctattgcgagctccctccaaatattggtgggacagttaataaaaacgatctgataaaaatagtaggtatcgagacctaagattggaatgatcacattcgcgtgttataagattggagatgttctaacttggatgaaaatgttagttacaataaccatatcctggttcgaagagtattgagatggactttcgacattataatatgatttcagaaaggtcgcacatgactgatcctttcctctgcaggtggtcctgtcatcgggtatgtttttttcctctagataaatggatattgtaagcaaatagtaattcctgcatgctggataccatacatgatgtgaccgccataagctaaccagcttctaaaaaaatacactccttgctagtatggtgattagttacggtgcatgaaaatagtaggaacgctgattctcgttcattttgtgtgcgttccacgacgaatttctgttcaaagtcctgcagatcttattgagacctttacagcac

SEQ ID NO: 313 - DNA, 2000 bp, Artificial Sequence
  Exemplary stuffer sequence
tcgtaggctaatagaaacagaattatcaattccttatttaatacatcactggactgagtcattctctcagagcaaaaggtaatcgcttcattaaggtattgtctatcctgtaagaacacccacgccgtggatatatctcaacatgtaattagggggtacatgcagtgtcgcaaaattcaagcgcgaactggggcatttctagttatgctagctaatctactcttgtaaaggagctttcgactaaaaactgccactataatctgattcaatggtggtaataagcggtaatctttaaccgtgtttttgctgtccgacttagtgaattgatacgtttatagggaaaaaataggtcgctcaatataccttaaagataatatcaccggcatgcgcctatgaggtatcgatcctgtgtctatgaggtaaaaaacgagactaaagtttgactgtattaataattatgaaagggaaccttgtagtcaaaagattaagagcaaacccgtctttcaatgacaagacatacattggatgcctcgaaattgattattaagtaaccagaaccaatgattatactaagagcttattcctttctccgcagactcttaagaaacaaggacaactgcccctgagcaaccagcctgctgatacgtccaaacaacccgttatcattagcctgtattgagctaaaagcacgtttattacttacatggcaagtattatttattatgtggctcgtataggtcgggtatagaaatgttgcacattacaagaaagttcaatcataaagcgaatcgtttatgttagcagactttatctacagttaacacgaggctagcgagatgtgctacttttcaagtgtttggaatgcatccgaggtcactataggcaattctttaccgcgatcaattcgtatttgaaacgcccggctagcctcccatagattcccagtcaaaggaatcaaggctgcgccattctgtgatttactccctctttggacaaccaacgtactagcctgcaggatacgatgccaacattaatttttataaccgtgagatcaacgcggtcaaggaaaaagttaggcataatatcgcggacaccctggcgtgaacgattaacatctgcgggatatgaacatttctcgatttactttaatgatacttggcttcataataaacataatacatccccctgaggttgataaacgttagaaacttaggcgagtccataagcgctttaaaggatcttttatcacacacgcgaaacattaccattcgataaaactcttatcactcatcccgaaatgccagtttcgcacatgcaaaaataagccttcgagattggtcacgcccgatcagtcgtctttcgctacctaacctatgataaaatagttcttaggagtcaggcaattgacttgcctgtgtctctttggaggcttccaagttcggatttaagggtatatgcctgttgtagtcggacaaatagataggataagcgctttccaggcggactacactattagtaactatcagcgaatataaatgtactcggcagcttaagcgtagacttagtactcgcaggacctcttgctcgttctagcatatatcctggtcgtttttaacattttaagctcgaaaaagttgtcggaagatgactccattagatggacgattaacgaacaaaggtctgtgaatgacatacacatctgatcagtattggccgcattcgcaggatagtacatcgcggggcagacgtattaaatcaacctctccacacccgggtttcgttttgccattgttgccctcgacagcagcgtttcattaataggaggctttataatacgtccagaaggtgtcagaggcctacgagctcacgaacgtatcctcataaacttattgtgtcaccagtcaagtcgtattttatctcctaaaacgacttacccacaccttatggaggcttagcgatcgtgtatatatgcttcttattatagtgcaccctgggttcta

SEQ ID NO: 314 - DNA, 2000 bp, Artificial Sequence
  Exemplary stuffer sequence
attgggcatttcgtcggacactaaatgaacattaaaggattgatcttagagtgctatattgaatcactcagcccagtccttcggacttccttgtatttcactgggcgtatactacattctcaaaataattttgcgagtcaattaaactagataccacctatggggggtttcgtcttggtttcaaattagatggtagtaagtttacgtgaacaccgttgagacgtagacggcttttatgggttgtctgtgttagactcattgagctgctcatccgaattattcattcagtactatttagcacttggacatccctgctagagctctgcgaaatgcggtattaggtctggggtgacctccagctcaattaatttacaccggtagtaaccaaaggttagttaaactcacgaaaatgatactcactgttttgtgtatccttagttatatgtcggcggattcaaccttcggataataagtaaatggtctcagatcgtagctgcaaaaaatcgtaaagcaactgttgttaagattggctactcctaacaaattccgcctccctcaagcaggacacttcggaatacaatccggaaatatggcgtgaaccctctatgatcgactgattccaatcacggttcagtccactctatctaattaacttatcgggtagatactagaaactcactcaaaccgtattcgtgaaataattattcggagtcagtaagcaaagcccagtgtgtattttacacttaattggctctctgtcaacttcttgcaaattaatccattacttgataataatatatcgcgttcaatggcaagaaatccaccgcagaatcgcaaatggactccctctcatctaggttaaagcaaaaatgttgagattccacctaaaagtggatatagaagacaaaattatttgtaccaacagtaaacagggacggaaggtgcctctcaggtagttactgaatacctgttagacgggttctgcccggcttctatgacttgagattatgtggttctacagtatatcatccgtctaggagtgaacctaatgaaaaatactctaggttggtacgtattcattcacataaacggatgcgatgagttggcgggttggaagttctgttaatgtcgtaagtacttataggctgacaagaggtaactgtcatacgaaaggattcggtctcgacggccgaactctaaaaggtctccttttccggagaacacaagactcttctgcttctgaccgtatttggatagatccatcggcggtacctttgtttgttggatcgtaacatctcttttgatcctactatgtgccaactcagttagttcgcgctgaattaagattcaagatcctgttcatatcttttataaaacatgtggatgtcttaaaactcatctcttcaaacgccattgctcgtttctggagtgttacgggttcggagtagagtggtattggatgtcaatatgtgaatttatccactctgacatacacaacgagtccgagaattttagatcgtgcctccaaacagcgctcaaatcttacaaatattaatgtagagccatggccccatgcagagatgttacattcgcatggatcaatctaagtttgtacaaaagaaaggcacttcttaatctgaacttcatatcgtgtttccctagcgattactatgattctagtgtagcgttagttgcttatgctctttatacactcgaggtatcatgtaccaacaacctagcgaaactgatactgagaggttgcagatagtcttcgacgatttagctactgtcatttaacattcctgcctaaaatagcttccgtccactcacgtactggatctcattctccgcgagccttatagagactggattacgtatattcaataataatctactctagaccaccgacctcatcccttgtttattgatagtggtgtccctagctgaccagtcttgttgggaagaagcatgtaacattcctattagcgccaacaacgcgt

SEQ ID NO: 315 - DNA, 2000 bp, Artificial Sequence
  Exemplary stuffer sequence
agagaacgtgtcacgtactaagtgcaaaagaggctgggttttttttgttagcttaaaacaccaatagacacaaatccatggagatttaaatgcaattattaatcttgatcgaattgtcttttagccgacaacctgttggtcccgacaataaatttaacgattgtttttatcctaagatcaaccgttgacgaacaaattaggcgaaagttatattagtagccagacgcgtttggaaacaggcaaaaactgctagaatacccgtagaaacctactggaataaatgaaccgatacgttaccgtctcaggaactacttaggtttgatagacagtggaatgccatatgtcttttagcgtaacaaccctaaaaccttattattggaaatttaccaggtaggatgtcatgtaacacgccaatccaattcatgtcacaaagtgattaggtatactagcatttataacttgggtaagtgcatctcatgtaagtaccgatgggcgtacctcttcgatgtattaaccagcacccacttcatacaagttcatcggtaagtggtttacaagaaacatcataaatagaaataacacctcttcagtgataagcggaaccccgtgccacttgaaacaatctctcgcagatgacccttggaacagggctgacagtttgaagtgacagggtgaagtcattcctttacaatttaagccgggaaatttatcaacactaaacgtaaaataaaattggcgtactgcctggacattggtcgcaatgtaatcttctttgttctcgtaaaccaaacaataatattttgaatcgtattatattgcacaggtaagccactgcaattaaattagagcccatcacttcccgggctaattgagactaagtcaaattatcctttcagacttctttaacctaaacatgaagagggttttggaattgttaaagacattccatggggtactgacgtagtaccagccagagttcgattcttacaattcacacgtataggtagagggtcccacagctacatatcctatcctgagccgaattctcgccattgttagctttaaatatttcgagccagacctgtggaatttagtgagttgaagactatgggagccataccgaagttgctaataaaattgtttctaattactcttcgtacatcagaggcacgccatgtgtgtgattaattcatcttgtttcccgtacaagcaatagcaatattgctcgcatcacgtccaccaagtaattattgtatagttactttgaactatatctctgtagcatttcgagtggtgctcagaggcgcggatcttgcctgtcggggattgtgaaagttggtcagaaagttacaacggtatggtattttagaaatcgcgaacctgattgcgtcctaacgcgatgttattagtattcaacggttggtcagagttatatacccctagagaggcctatggagatagacagtctcgcgtatctcatcataactcttgatcaatctagtcaagtagttcacgggactagccgtacacaataaggaacctaagtgcaaaaccactctttagataaggatcctgcgccatgctttgagccgcagcattctctcgatgagtccagcgtggtttgcaacacttagtacataagatagttaaatacagagcggtcctattttgaaaaagaaatcctatggaccgcaccagccggaggttacctaagacttcggacgaacatccttgtttaaatgtatgactggatgactgattttcaacagagcgaggtccaagaaaaactacaagccacttattaaagacatgagtaaggacgagttattgaaactaagacatacgtgggatagctaggtggcataatacaagcagataaccccgtacgattcaaacgatcttaacaagtattttattacaaacgggcctggttttaagagaaaaacgtgcagtaccctcaatatgagtaataagggaagtgacagggagcactcggcgat

SEQ ID NO: 316 - DNA, 2000 bp, Artificial Sequence
  Exemplary stuffer sequence
agggcttgcatatccacaaaaatgaatttatctaggttcaattacgtgttatccactccagcgaaaacttgacactaggattattgtcttttgtcgacacgttaatacagcaacgtccaagagatctcttgctttggcttgaacttgcaatattcacgggttgtttccattcttacctcgactggctagctgaatgacctttcacctgggttacgatgtacgcggggcactgtggcattaaacgaagtcattatctgcaccaacccttgataacaaaataaatatggtctgcgacaccttgtgctgggagacaaaaatcttctgtaattggttctgtacgacaggattagttcctctttatttcttaccatgtttcctcttccagcattaagatggtaaattgaatgtatagtgcgcgatacggagcacgtgtcagttgtcgctcggtcgtcgcgattattgcttggaggatcctaataaagctaaatgagtggagtagtagtatgcgtgtgtgccggccgtaatatctcattcacgtgcatcatagcgcatatattcgacacttgtaatcccgtctttcgaagaatctaggttaaatggatactactttttacacacgcatcctgcctctcggcgggaaatatgttattagaaacttctgaagttgtctggattaaagtactcatcatggctaaaacactctatttttggtgtgaatatagctctatttacttctatcgaggcctcgttctagaggttattagtgacagtccgtccgtaaattttcctgtatactcgtcttccttattagggttgaggtgtactgcatgtcttatgctatacaatcagcgtacgatcaagactgtaatatgtgtatacgaccacattatgaatgagggtaaggtgcgatagtcagtagctgcttgctattatccttaaatcgaataatgcagcgcttcaacaatagatcatatgtatttcaagcaacaattaggggattcaactagagatgctaatgtaggtttgtgaatattttggtcgtacattggtagggcatctgattgcatgtatacagtcataattcagagcgacgctctttttaaccttgggaaaggccgtgaacgaatgcgattaggccaatctagcgcatatagttaattattttactctttatctcttgagcaacagcggcaaggaaacctgggagttgctagacaccgagtagaaatcccttacttcgccagcggatcgatctgtactacatgcatcttctactaatggttgaaagtgaagctagtacttatttgcatggtgcacccattcttacaaccaggttgttctaatgtcttttcatcaattcttagcggagtgggcataatgaaagtataagaatggaagtgttctattttgcaaccggagaccacatgaaaggatcgacacagagatgcaaacagtgcatacattcgatgtggcatagaccaactcttgtacgatttaatgtgatctctgtcacaattcgtttaggtgtctatggtaaaacctcagccacaacatgtatagtcttacaggcatggctatcgtgatttaaccgtgaataacttgtcggtaacagaaactctggcacaggtgagcgtaatcaaatcaacttcagtaatgaggacttctaagatagttccgaatctgttcacagtattagcacggtgattgagttctcttctaatattcctatctttacattgcgtactgtcacagaatgctgttgcctctatgattttacaacggcaatctaaatcgtcgtatcatatgttcagaatattaaatagctcaactccgtgttgagtcctaagataaagatagaaacattgactataaaatctatccattgtaaaccagactaatcatgcaagcacaaattagagggcagaccgcggccattggaatcatttatatctttatcgtttaattcacaagaatggctaaatgccggattttgaccgggc

SEQ ID NO: 317 - DNA, 2000 bp, Artificial Sequence
  Exemplary stuffer sequence
ttacataacgactccgtcgaagccgtcccggacatcgagtctgacacttacaaccctgagagccgcttccctatatgtctatagattgcgagtgtatgccactgtcattgcagatttagggtcaccccaaaaacacgagtattattagagactacgaatcatttagcaaacaatttcgcgaagccctaattgaaaaggcaaccgattcacccctggatagataagctaaaatagtgttatgcggagcaatgttctcatttggacccatacactctattccttctgaatgaccttcgaaatacgaataagaacatggcgttcccaatcatccatatacccgttcaggctgagtagccaacatttcgtattcaaagatacagttgacaagctgacattcattgatgacttaggggctaacatatcaggccttttcttaatgtttaaatacttgcctattatgtggccatgaggagtgcgatgataccaatgttattggagtatcgttaaaaaaattcggtagtgttataattacgaactatagcttacgggtcatctattttaacatagtgagggcttcttcacacttccagtcgtcggtctgcatgaaacaaaaatgagttacatttagaggaatgcggggtaggcacaactaaacacaaggattaaattcgtcgcgacaggagtacactaaacgtaattaaaaagctaccaggcgaaacttctatttacgggcaattacgaatcctatgacacttcaaggacctctcattctaaaatagagacagcctccactcgagctccgattgagctctgctctcttccaaacaagaacctccgtgcgagcagcatatagcgagcattcttcggaaggacctatatagatcggtcagttgggaaatcttacaaaacgtcgagcatatattatttgccgtccgcaacctatgcacaggggcctttaaatcagtttatttaaaaaatctaatttcaaacagtcttgcaataggttaggtgggtatagagtatcaaaaatacgtgactaaaaacaacagaagttgataaacaacagtgattttcgggatttatgctacaccttagcgagaaacttctgttaacattgtctatgctttgaaactatgtaaaggaattcgtgatatggtatacctaataggcccataccattaaactgaatcatagtggacgagaagctttatcgccctctaatgcgtagtgacgaatgaaaatcagacaaccattatagaagtccgagtcagccacggatgttcggaattgctatatatacgcatgacttgccaaagttgtggtttactgtatatttcgtattccacaattacatatagctaaatctacgatcgcggcgcggtataagatttcaaactcggtaaacttgaatgatttaaatcatccaattgttttatggatcgtggcctggagtttggcaattaattaaaggatatttagctgaatgtgtaaaataatttttaacccaaatgtgtctataatatgtgctcggataaagctcaggcataaccacagatctacgcgaccttgtgatcgtccttgtatgtgtatatagagcaactaccaacagttgttcagacgcaatcaaacgatagctttacgataggatgttcatttattaccaagtactattattcactctatagggttattatatcctctactactccggggtgcgcaactttccttacgccattattaacggaatgagcggtaagcggcaccttctatatcatcgtcataagagtgagatgtaatgttactatgccttatgcttgccatggtaagccgaaaataagaagatcacaaaatagcaccatcttttccatagattctcataaacattgatgtttgagcaaaataacagctattacaatgatgtaaattattataaatgtctaatcataagccagtaatttcgttaagcaatctagagaagtatcttaagagcgttaagaacc

SEQ ID NO: 318 - DNA, 2000 bp, Artificial Sequence
  Exemplary stuffer sequence
cctcactgagaccaattatgacttttctcttgcaattacacaatagtgcgttaagtactgaaaaccatcctcaaggctaaatgttataagatttttcatacgagtggcgaaaaccaagtcaaactggttaaatgatgtctactacaagtttgggcttggctgacaaatttttctatgagctactgtaataatgcgtcttcatacgaacgcactctgcccataaataggcgatggacctaatacgtcaagcccatcttcaaatagtttttcttgtaaatttttgtcttgacagacatgatacgttaacgttgtctttgaccattatatcttcgcgatagggtcgagttcgtatttattaaattgatgaaattgcgacacatatcacgtgacttaatcccgaaaaattagagttcttgcgcttgtcataggcatgaaaagctcccctcataatacgtttgacctttaacgtatgtctttaacatatgttcctggtaaccaggatttaaagtcatggtcagccttcgaaaaatgtgagaagatcgcgaatacatcacgaactctctcaggcaaacatctcatccaccatttatatagtagatgcgctacccactgttaacctgtttgagatgtcgatttaaacgttagaaggtggttccatcgctggattgcaacctttacttaaggtcgatgatacgtacaatcgctttactttaagctaagttattggcatactactgaaattcacttcctggcagacttgcgttgctctcgcaatcccgcagtcctttatgatgtctaggcgttttacaaatcgacagtcattgtattaaagtcattggattgtacggtgtaagtcgacagggaacgtgttgagttaatagtaaaaggttcagattcttgcaagcgcgcttttctatcgcctggtttatcaaactcatggtgattatatattttgcaattcatcagccctcatatgttggtaagactcggattgggtcgacgccagactaacgtcataaatgttagaattattaaagacgcaattgtttatgatactcactaatgggtcgttagatacttattgttttaaggcaccagcctccatttgtccgagtccaggcccgagcttgggcgcaaaacttttagtatctaactgtgagtgacaacctttagagttctctcgtatagaaggtccgacgtcagagtatcataacctactggaattggccgggttcgcgtgcactctcacttcctgccagaacgcaattaagcatgctggtagtctcgacccggtacctcactctatcaaatgaaactatagtatacctatcgatcttaagatgtgggttctagctgtgactgcccgaagaaatagtatttcaacgacccgatcgtctaggagcgttgtgggagggttcaatgctctcgtatcgattcccaagacgttgtggacatactagctggcgaataatactatgtgtagtgaagtttgcggtaatctgcgtagtggctaattaagaaacaccgagccgtgtcttttgcaaactcatcgaggcgttgactaaaatgtctaacggttagggcgatattttatttttacccgcggtttattatctatgagtactccccattcccatatagcgtgcatagtttacttttccatatgttattagcaggctgtccgcccaaacgttgcgctagccaccgttagatcacagtcatattatcataacgattaccaggttatagtttcactgactaaggagcccataaatgttcattttcactagacatgctatgggtttggcccgaccaagattgataaactgcggtaatggcgatatgattaaacgattaaacttttaactaccatggggagacaagacttcttaactagtcggtatggattgctgcttgtaaagctaaacaagctgaatgtaagaacaggctggccggttcataacactatcacgagtggctgacagagttttacttatagt

SEQ ID NO: 319 - DNA, 2000 bp, Artificial Sequence
  Exemplary stuffer sequence
attcgcattgtttgagtagccgagcactagtgggatcatttaccttctcgcggaagagttacaaaagtactgaggaaatatgtgaattgttatagcttttaggaaagtaaacatgaaacaaggtagaacagatgacgacgtgatacaattatttacacaactggaaaattccgtcaaagttttaaagtatattccttgagtcctattattgaatattcgaaaggtagtcacctgagttgtcccgtaataattacataagtatccgtatggcaacaaatatctcctagatccgggccgcggatagttttcgctaaagtatctaaatcgaacttcttagcatacgattactagactatcaccttgagtagtctatatctctgcgagtgtaaaatgcacacgccgttaaatcgcctaaatgcctttccgtggccattatatgccccacttgctttcaattcattccataaactatgatcatggacccggttgcgagatgttacagataaagtcgaaactttcaagagcagctgacgacaggtaaaattacgatgcactgcggtgtaaggaaataatctccaggttgcaatagacatttaaattgtagaggaatagagttacgcaaaccaagcccaaggatctaccgaacccctctaccttatacaaactcgtcagccgaaatataccaaatagcacgttgcctagaggtttacattaatcattttacacgatccctttactattaatatatcgattccgatctaaaaggcgtttcaaggatagcaatagtcctatcaaaatcattcagttactggcaatccaaccaattcgctgtacacgacggggtgaggtcgtaaaatattatatgtcatagatgcactgtttgcgaccatgtctagcatttttcaatagctccacccacgcgttggcgacccattgttattcaaaaatgggccgcatgaagagttaattcgtcttgttctgacataagtgttgaccatcagacaatagacgtataccgctggttacctctaatcgaagatccagagctccttatgcaacgtatagtaaacctggctcggaaaggggttactcttatttttagcacctacattcgggatcaaatcatatgcactttcaagatggtgctcactataacacaataacttgggtttccagttaggatgaggaatccgccaggttactctatgaagtcaagctcttccgtagtttaggcgacgcttgacccgcgttcctcacaagtaacgcgacagattggagcaatagcgactgcttcaccatatagggacttacatacagatcgaatgatttgcagctttaacaacccataacgatctgcactagatgcgatgagatctctgtaaaacgaaacttggaattacccagagcagttctaattaagctttttcgataatattacacagcaactaaatgagcacgtatgctcaagtgtcgcaaaatccttattgtataggaataggtcgttgtcacaacataggtctgtcaccaaactcagacattatagtactttacggagcatgtttagacataatctgcacaatgctgattagtctcagtgtggtcaaattctttaacgtctctgttccaatcaaagtgagcagactgattgcatcacaactccatcacttaaccaattattaatagtccacacaattcattcactcttcactgttcagcactcagtcatgctctggatattccatatttccccgccacatatactgagtttggtcactcatatgttcgctaaaatcgatttttaagccattcttgcctattaacgacggtcctaatcgtttcccttcaccatggatatacggtacgggccctattatctgcgttacgcaatgtcaataaaagatattctaagaagaaaaaaagataagttgcgtaagcgtgctgcaagagacactctctcttcgcagtaaactaatttttcctttaagaatacaaagcgaaca

SEQ ID NO: 320 - DNA, 2000 bp, Artificial Sequence
  Exemplary stuffer sequence
ggattagattgtgccataacgcaacaggtaaaattattagaccagcaaaagaatcctaacgtatacaattttatcgtacataacccgtgaatcttattaaacccagccaggccgccttactttgctccaagtaggagcataatgcatagaagtttcagtatcctgtctaaagctattaagtcgaaatgagacaaaagtgacgagttattaacgatcagaaactagtctaaagggaaccctcctgcggccatttcttgaggacttacgtgcaccatatcatgaggtcctactgtgggaaaggaaatcctcagtttacatgatttgaaatactgtagtgacctgtcaatttactgatttctatgcataaaatgacaatctcaccgagtacgcataaatcagcgcagatctcatatattcataataatctccgggacgttattaaattaatttttttctagacagatattcagaagtccgacgttatacaagtgcccagtaacatgttctgagcaaatagattgtcgacagccccaattaaccacctactagtctttaggcactgtgtgaatgaagctattaagtactagacataatgtcattgctggctctagctgaagagtatacctagctttttttccagatttttgagtacgggatctgttcttgttgaacaaataatctggatggcgccatacaggcgtcgcctggagcgtcaagctcacataccctatcgtcaaagtatgttccgtcaaaggtgtctcagcacttaaatacttaaacaatccgagtttcgagttctaaatggttgcacaatatgcctggtagattgatataatcttgaagcaacgatggatgaacaaaaattattgatacttacttttacccacacaaaccgtctgagtgtctttttaagagggttacgaatatataaaagcggatcacgatattccaccgggaatagcgcaattagtcatatggaacatggtgtgaaaccacaactatgaaatctatccgtacaccaaccaagagacctaaaagttttacataatccgtttgctttcgtattgccctctatctaatgaaaacccattgacaattataaagaacaaaggttatcacacgctgcgtatttagagaagagaggacatgtgggatcaatgtggtcgcaaaaattatcactttaatcaacaccgattctaagaagaaataaacgtcgtattcaagggtactgtataggtacgttaagcgttgtcgtacactcagcgatttaactaacagccgggagaatgcataattatgataaagtgaatccacttagcgtctcgaatagaggctatttcgcttgcaatcaaatgcttaagagtatcctaaccaattttagacaaatatcagtatgtttatcgattaagctggacaattcctctacacagatgtttaagcgaactagcattttcatcctcccgactcataggagtccttcgttgcacagtagatagtcagcgtgtgttctcttctccaattgatatgctgaaaaactataggttacccgtttcggtcggataaagaatttgacttaattttcttgccgatagtaggtatactgtaaggcagccaatataaccgttagagcttgattagtatgatattcgctccttttaatgtatctacatctagctctggaaaacccggtgtagaagtaatgtattaagtctgcgaagcgggaatctgcttgtgacaaagattctgtcgcccgcaaacgtcaagtaataaatcgcagatacggtcagaaattccttctgcatttcaagattagtaatctattcgattccaaacatcctgctcctaacagaatgcgcacgggacctaatgaacttttcatatacgtttcatcaagcagtagtgttcggaaacgagacataacagggtacatgtgcatcaacctttaaaaaccaatctctatttggtatagtcgtattcgaaatccagtagtgaggtgaaaa

SEQ ID NO: 321 - DNA, 2000 bp, Artificial Sequence
  Exemplary stuffer sequence
aattggagccaaccataaattggatggtagttccaaaattttataacctattctagtgtctgcaagtatttaggagataggtgaattacacgtcgtacacataaatatgataatgcgatcaagagtgaatggggtctatagtaatatgatgtaaaacttaaggatattgtggactgatttaacgttacgtagtcctgacaagagtttagatgccaggtcgtagaagttgtgtatccccctattctcccaatggtagataccgtgataaaagataaattcctgttaaggaagtcgaggatgttctgtggagtgcagagttctacatgtgatgagataacctaagagaaaaagtaatttatagattgcccccgttaggagctacacccgactatttgtttcgttaagatatttgttcgtaccatgctgttataacgacactccctcgaatcttattttatggcaattaaagatgttacaggtggcgttggcaattctggtaaactccgcactttacaaattgttgtttgcaactctctcatattgtatgcaatcgaccccaaaccctcatcctcgaccctatgaatgaaggttttctgtgccaaaagccattttactcaaaaattagcttttaatttggggagcttaatagcgaattccagaatcgtttcatggggattaggagatatattataggagtccaccaatagtctattgacttagtggttttggctcatgcacggtggacaaaacttcaggcgtgttatctaattacaacccgtattcatacatatcaggggtgttgatttcagagaatagattaggaaactacgagcaataccaattttgaagatatggtctactagtagctcacttactcaacattgctactttattcgaaggcccatattgaggaatactgtcttgttgagtaaaacgatacccgtaactttaaactataaaggcataccagaaaaagtgtcaccgcaggaaaatataagaacgtccatcaatatatgatgcaaactagagaaagagcttgataaattatcaaactagcacttctgggaatactccgtggttgcaaggttacagggttcagtcaaagagttattaaatcgattgatatacttattcaagtgattgattctatatagctacgcatatctgctgactttttcgaaacgttgcctggttgtccagagcatgttttggacgagaaatttcgcgcagatatcatgattacgattggcaactaaggatgactagcgtaatgagaacctggctaattttgtgtttcttattcaaattgtataactaggtaaggaacgactcgttcagaatgagttctaatcataatcttctaaaatactgacagaaataataatatatattatgactattcagaaaacctataaaaagcactccgtagaagctcttcaatcttagaatcctcacctaggaacctgaagattattgtattgacttattttgtagttattaaagaaatccaacgacggggacgactgcttgtatgtaatatttccgttccacaagccgggagtaataataagcaaccgtagaggagcaatgggtttttatctcacgcacaggatgtcggagtagcgagccgtctgagtatgttatcaccaaagatatatgtaatatggttaatcagctgatttaaagagaacttcatcccaacctcgaccgacgatccgattactgtttatcgtcataccttacgagatgtcaggtcctcgcacaaaccgccacaaattccttgtcactgcaagaataagtttgtccgcaaactgtctacgcgctaggtcgttgtatgtattgatgagccctatccttatgacactcggactgctagccttctgagatttacgacaggcagtctagtattaaacccttactactttttgctgtatattgcattgcaagttccaacaagttaatgaaacacaaaccgtgatcgcctcaccccacaaaaggct

SEQ ID NO: 322 - DNA, 2000 bp, Artificial Sequence
  Exemplary stuffer sequence
gtaagggtcgaacctctgatcatattcgattactaataactccagatatatagaattgagaaaggcaaatgtattttaaacagcaagaaactgtttcaattcggcttatctgatgtacatttaataaatagaatgaagatcgagtattagaactgatatgaaagttcgtaacatcaggacgattagagtttatgcatgctaacaggaactgacctgctgacattatatcatacaatttcctgcgtcccgcttatggatggcgtcaataggctagtaacctaattgcagcttagaataaggagaaccaagtaacgacaacaaaatgaaaagcaatagatggcggactgcgctttaattgcattgaaatactctgggcttcaagtgttagttcattaaagctgtctcgcgatacacaaacgctgcgaagtggttccggagtaaatgtgaccaatgttagacagtgggcccgccatgaatgtgaagttagttactaggaagagtattctcagtttggtgtttactagaggtgtgcttggcgtttatctgggataataattgtaactcaattctattctttttcgttttttctgctcatatcgaagttttgctcgcctcaatcaacgttgtttgtatagcacttaggatcactctgcgcatagggaatgcttaaatcagggagttcatcggtgtccatcctgcagggacatgaaagctgtcatacacggactcgtaccggtctgacaatccgctttgcctcatagcaactattgagccgcattcgcgtggagctgaactatcagaatggctagaaaggataaacctgtggtgggtccacgagattggtcttcttatgttaatattagctcacaaagtccagagttagtatccatctcttccagtcacatggaattttactaattattgtggtatcattattataaaaatgacattatctagcatgactccctaccactagtgcagagctactatgtacataactcgctgtttatgcgatactccaacaagtagatacggtaatttcgatataggatgaaaaaaccttcataacagcttaagtttaacttcgagggtccgtgtaatcggacaacgcacatacgaagtggcacgacctttcatttgggctcccttttgcaggctagtaaacctagtatacatgaaagccgtcttgcttgtgcctacggcttatttcgttgaacgtacgtctaatagtgccaaggaacgaacacacggctagatcataatattactccaggtgatggtttcggtatttgcaaagtaaagataagttatctgattcacaacaatcgagaatttgtcctgtttgaacgccgaaatattatcttactattgctttactcagatacctccaataaattataaaatggcttgtttgaatgtgtatcgaaaccgaaagctatatcttttgaccgaattaaccaaatgctacgcgtttgctgtttattatgtccatcatcgctttaggttaagcttaataggttagggaaaactaccagcattcacataatatcctatctaggaagttaaattcacccatgtatactatactacttagtctacaatatttctgctttattctttatttccattatcaaagtatttcggctcttaaatggggcaattacgaaagatatgattctagctcatgctcaattgagatgaatttatgactttaatggggtgtaccatttaataatgcagcgctaacataacgtgcgacgctaatatcatttactaatagattttcattcactataataattaataatcttctggccccatggcacaggcaattttaaatccgtacccgtcagccctaaaatgccaagattagtgaatctggtgtcatacaggactaacaggtgcaaaaaccggttgcgtcatcaaaacgcaggatttactcaggatcttaagaaatctaaattttcgcagaatcgctcatcgccaaaattttaggcgtc

SEQ ID NO: 323 - DNA, 2000 bp, Artificial Sequence
  Exemplary stuffer sequence
cacgtggttttcagcggttaacgcaatctgcattattggtagaattttacacttaacaaaatatcaccacgcggacaactgatttagcaaatgccgtccgtgacgcgggacccgcagcacattattagacatagtacatcagcctgtaaccgatcagtcatcacatatcccggaaagatttcaatccagttgtaatcaacgcgtaaagttatataatcacttcaatcaccttactaacttcagaatggcagcctaaaaatctgatgctacgaaccgcatggtgttgaataaattcaatagaatggagctcctggatatttcacgacgccgggacagaaatagtgttatagagaagaaggcatgccgttttactcgattcgtaagtagtttgacgaagcaaaaacttggggaagaacttatgagttagccacgacaactaccgggaggatttgcttttcttcctccatgccaatcttggagggagtacctcaatcacacgatgaatcagccttaatgggcgcccaaaacattcttggtgccagaaaagcggatgcttcctcgaatgtgtaatcagaaaagtggtagatgaatctccggctccatcatggatagagctgcaggtattggtgcagcaggaacgaaggttctaccagtaagtaaagtttgacgttagttacgagtctagaaggcccaaagggcaaccaaaacgtcggcaccataacatctacaggtggtaggctaatgtaaaagtggttataattgctaggcagaaataaggccgttcattgggcatgtgtacactccattgatggagcttaattcctctcaaaataattacattctgttaacaagaaataacttattggtcgatctacgagctagcaataaataatcatgaccaaagagctgtgctgtgatcagaagttatgacgcttatacagagagcattgtaaagggcaggccgaagcaaattcacagagtacctgaagcgaacaaaggaagagacttctttataatttacatcgcttggcaattaaagaagcgaaacacagttgctcgaatcacatccttacgtgtcgtcgacaatatcataagcattactagtttagagaggtgagatatcggtagtaggtattagaacattctaatacctaaagctcattactattagcacctttcctcaccttatttggatttcccgcacgccgttcgcaccgagctaagtgcaataagccatggcgatgacttagatgtcacattgccccatgaattcaccccagtgagttgagacgatttgaagtttaatacgtcgttcgtggacagcttgaatgtttcacacgtggtaagttgcatatgaacatataggaggggccacaaagcttatgcgtgaagcaaatatgattcctccctcgatccgttaattagagttgctgaagggcataaactttagcgagtttgtattaacatagtcatatgaagtaacagagacccgtcataacgcttgaaaacctgaactcagaatgcgctttgtgtaccataggcatataccccacattacggagatgataatcgacaaatgctccaagaagtagacctctagccatcatcacgtgtctctactgtattctccgaagttccggaggccagttcttaagtaggcacagaacacacgatggatttcctagggacgtacgtatgttcgacttctcgtcagtaatcgcgacagaaatgggaaggtgagcttaacctaacccacatttttgtcatgggactctgtgaatggtgtttcttatgaagctatcacggtgtaaagatatctagacacgctatgtgctactccgataaccctacgtttaggtttacgagattggagaaatatactttattaattcttccctggaatcgtaccaacaagttccaaaatggctctgcggtctgtcaaaatatgaagggctcaacttgacaggacgactgaccggaaatgatttaagtgaacctcc

SEQ ID NO: 324 - DNA, 2000 bp, Artificial Sequence
  Exemplary stuffer sequence
ataattatcgacatagatgtgcttcactcgatttgacagctggatagtaagaattagtgtataacccaatacgtatgctaatacaaaccctggactgatttgaatgtaatcctattcataatattttagctaccgtaaatgtattctgcaattgaatttcgtgtgaatgtaaaaggtttagaagtttcctaagttatcgggtgacgtttttaatgggtcttaccgtagattcagacaatcttttggaaaccaactgaagaaggaaatcacacgacctggcggataagggtttgtaattcgcgttaaaaaactgacgtttgctataagagacgttaatgtaaatgtaacgctttaaattctctgtgcgagagttttttaaatgagatcaaggattgttaatttcaggaagctccgttattggattttgccttctcattcgtcactatccctctccgatcaatccgattgagtcctagtgtagaaagttcacatagaaagcagttttccgattagtctagcggggtactaagtgaacactagtcagttggtgatatactatagctaggctgtgataatgttaatcggtttgtgcctactggaatgcttaatttcatcttgaggacttgcgctaggaatcggtatgtcttcgttaagtccaaagtgccttttcgacagatgttggattgatgcactcctccgaaaaggaatcaaattgggtttataaattttgtctttgtgacacctgccgaatttagatctcaccattatccacaataaccctattatctttacctacttccgtcggagcttgattatgaatattggcagaattatgtaatagtcattaatatgttgaataaagatatcaatacattcagacaattgaattaatcctgcgtaaaaacctacttaggacgagttgctggtatttgtttttataatggtagacatgagggacatattacgaacctctgtaagcctgttctgatgtggccggcgatcacgttacctgatgagatttatagatctcaagtcggatgtcctctttaataaactgaaaaattgacgactaagtgggctaattatgccatcagaaataagctaaccaaacctctaaagtcgaccctgtagtataactggcagtgctagatatcacagggtgtttgtctactgaaatttcggcattctggtcacacttattgccgataggttctagtagctagtttatctagactccaattgaaagcttacttcggcctatcaggttgaatgatagacggtctgtcttaagaaactacaggacatatactgcatcgaatgcgtttaaatcctaacgcagaagggttgttatctgatcatcagtaagcaccaatctgcatgattacagacgtaccaacaactgaatacatcctgcctcctgagaactagaacctattgtattgcggatgagggtaagataggtagaaacctgctgccaacttatcgataataattatgaaccatgcgtgggtgttgatatagacttaatatgacctcctgtctggttcatataccagttttcaatgcttaagagaactagcttgtacggagtttttttaatacaagtgctaaattaacaattgttcaaaaacagtttatagtagtaaggtattgtaccaatcgtatagcaataaatcatacctgtgtttactccatactttcttgattatcgggcacgagaagaggacaactcccaaacatcaatgtagccatagtgaatgaaaaaagtcggttatgaatcgttagctaaatcgtttgctccaattaacaaaactataacctaaactggtgaacacatagataaatgccaactcgttatcgtgttatgctatagatccgaatttggtggttctccgagtctgtatcgtttttaatcgagatcttaccttattcctaaccacatttcgtaagcctattgaaacgggtattgccggttcgcccatctggtagtacgtaaacga

SEQ ID NO: 325 - DNA, 2000 bp, Artificial Sequence
  Exemplary stuffer sequence
gaggttagtgatcaagcgcattagctttttactgcggaacgcatacaggatatttacgcttaaaaaggtggatttcgtatttattaagtattctctttactgaattattgtccatcagtaatcgctggctttatgaactatcaacattcggtgttgtgttaagttattaatgacacatgctcgacgttccccaattcccgtgcgtgatatattatcatatgaccattaaatgattaaaggggcataatattttgaaataacactattaatttgaaacttttgtccttttcgcactacatgttggtaacatcgcacgcactaaatactgacatatcgtgcaccatgctttctaatagcactccgttccagtccatagctgagactgtcttttcggacaacacaatagataagagtctatctctcatcaaaactgtaagaaaagctctaccataattggggccgaaacgtaatacgattattatgatatcgctcctgccgaggtcaaacaccatagcactcaaaaatggtatccaatttagaggggctatgagtagttaaaaaataggaattaaggtggcaacaggacagaagtcaataggttcccttgaaggctagattaacagaactgtaatgtgactgcctgtaagcgcactggagacatcaagtattgtacgagtataattgcactttggaggtacaacatcgcactcgactctttcatcgatattttttcgtgggtgaacttgagttaaagttgatggtcccattcacaacgagcggttttcgcgatgtaaacgccggccaaagacaacctaacgccgaattattctacttcatatgcctaagtaagcccgttctttggagaagtctcatcctctattattatacatagttatcatattagtctagtcgccaaagtgtggtttctaattgataaatataataagttaaaaaatgagagctcaaagtttttccttaccgtgccgcacaagtaagtagtctcaaaaggaccgcgtagggagggaaaatttaatgagttctaatataatatgcaggcttgtgaaagctgacattgactactctggactggtcggatagttgctagacatacctattgtgacaaactgacccattatcgagtctagtagaaccggtccgtacaattacacattcttcgtaaactagttctataaagactaaaaaaatctatatcacttggagaattatggaagatgagtcaactccgaagtgtggtcaaaaatattacagattgtatcaaatcgaataggccgtaaacaaggggtatacgttcacagtacaaaataaatcaaagccttcaattatatcgagagattattacactaccgctgctcttgactagtcaaacgtacctctcattgacaacattcagcatgattattgctccatgtcaaagactccgtgttcccattagttttaaaggcataatttatctcttttcctcttggataacgagagataattagacaatgctagtttcaccaagcccgactcgataagtggcggttttagcctacccaatcgcctaaatatatcaaaaatgacttgtacgcgataatactgctcgggtagttaacggccaagtacacgctcacagaacaacggttgtaccgcttatctaattagggaatgtacggctctctcactaatatgcgattaatctattttgatttttatgcagagcatcctaagtgaaactctagatgccgccaatttttgtttatcatttcgccaaccgtgaattccaagatggcccgccaaagggcgtataaatcgagtatttacgaagtaataagttaattctaaaattctttaaatatgaagacaaacaatgaattgattatgatttccagatatttactttggtaccggattaaacccatttgaacgtcattcgatatcaaagtccgctaataagggtttcaattacaattcttcaggagaacacatcggtaaccttc

SEQ ID NO: 326 - DNA, 2000 bp, Artificial Sequence
  Exemplary stuffer sequence
gcgcaaaccagcaaattaggtttgaccttcaacaactgtaactcgatctgcagacgagtgagtaacaacagctactggtacaatttttttgtaccgcagcattcaggtattaccccttcacgctcagtacagaggtatcgggcatccgtataaaaaattgacttctttttacgatagtccaatagaccgttagcttctacttcatagtactaataataacctaatgcaatagtctggataacattcacgggacactgatactagaatcaactacgctgatgagcatgtccagactgacaatcggtcgacatgagaaggaatagaaaaaatcctaccctgttaattctggtcatgtttgctggtctctttcctactcggtgcttctcaaatgccacatattcgagcataatacctagttataggcataaacttattgttgctgcccatgttgagcattttttatatttaggccttttacgaatttctgtttctattactaaagatgtcagagtaataccaccttcagacagaatcacatgattaaaactatagaatcggcggtacaaagatgtatctcacctatagagtatgctgataaaatcatagaccctagacatactattcttatcgccccttagaaattattgtaggggttgcgattacaacgcatacggtatttgctatatgagcactcatggcttatgtgtacaatttattgatatatatatttagagctccggatcgggttacagaatcacttcacgacccagcaaatgctaatgatttaagcgtagtatattggctttgtgtccagttttcactacgggttcctttctatgtcctgataatctgtacaaccgacataccctgaattcatgccgcatatgtcgtgttaacagtgatctagggtccagtgatagggtcattttcgtatcgtcgcatctgtatcgattggaaaagaattatacagtccgattatcacttagaactacacgaggggacctcttatctgccctacctattggagttaaagttctaactgctcaatctcaagacggccgaagatggttttaaaatgacggtccacacatttacagacaaattggaatgcttagatatatcctactgttgatttttgtccaaaattagaggcgatgtaaccccactgaaagattgagcagtacagtaattctaacttgaaaaaataaatttttgggtatgctcaatctttaaggtgacctactaacaatatcctagatcccatacggtagttcgacagagatccaatacattctaatcgaacattagtaagttaaataatatagagctacatttctaagtaaatcgatgcttgaagatattggtagttcgcagaatttgcatccatcacaaacactagtctttacgtttgccaattgctaggtagagtagattacgagtcaatcagaagaccaaattttttgacccataggatacaacacgtagtcatgacaatcgcatatcgctagtatgttagatctaagaaaatagtctacttaaccgggtcatacatctcagctattaacgatattatgttgccttatgttagacacgtcaataagtagagcatgcatttctgcctcaaataacaaatttgttaatatgcaatgaatacctgagttgaatgaacccaaactaaactcagggtccttccatagcgagagcgctaggctaacatgagattctgacgtcttcgtgagttgacaggatcttgccaacaaattacatatttgaataggcatgtacgatccattatactatgagtgccagagaaaactctgctggccgaccgttttacggggggaaagtcaaatatgtagtaagtacgaattttcctgggagactatagttgctgaacgttcttattctcattttcttgaagttaaggatggtaaaacatactatacctatgtagatattctttggtagtataactattatagtagcgtagacgttatgtg

SEQ ID NO: 327 - DNA, 2000 bp, Artificial Sequence
  Exemplary stuffer sequence
gcctaaagacctctatattttaagctagcataaaggcaggagacgttctaacatcgcaccgagttcgactatgaagagaggtattatcaaccctgtctcccagttcacaccggttgcattatcatgacgtttttgatttgttttttttgagtaacgggttcattgtacgttcgatagagtactcgataaacgactcattccacgcaagcctattttgtaacttataactagacattagtctatggctactttcacacccgaacttacgaacaacgagtattttttttttggcaaaaacgtaacgttcgtatgtggcctaagtcattaaaagacaaatattgaagaaaaacccatgatttaataccgataggacattacaagggtcattagagataacaaataaattaggcttcttccaagagttatccgactagttgtgctccagatctgcgatactgatcgaatttatacctcattagacattcgtagtcattggtgttggacttgaagttctgtacaatcctcggtgatcactcttggacaacctgctgataaaacatgtctatcgtcagtccagtttgtataataaactaatgagacaatatacaaaacaatccgtggcactacatgttgtataccaacataaattctgaagacctatgattcttgtggccgaatagtcaacagattttacgatcactaataaccatatatctgttacttgtcttctcagataggagcggactagaaatactcacttatgttattcttacgttactgtgccagacgagaggtttttgcagactctatggtttgccggatcttgctaggaaaagggtaactggtgcctgattgcatgaactatgtggtatgactatagatgaagcatccgtcactgagctcttcgaagtcttttatgagacaagaatattctttgatagaatcatctatgtctcaatttaatcaagggaacggttgggtactaaatcgagttatcatgaggtcctatcggaatgcattgtatttgagcaatatctataactgtaggtactatggcggatatttattttccttgctgcgacttcatgtagcaagtcggcaattccccgcggttttacattttctgcttcgaggtattaaggccctaaagttgtatatattataaattaaagatctggattattaactcagtgcagagggcgtaatctgacgtggcgacatgtagatgaagcttgcccaaaagatatgagatcttaatatctataagaagtatgcctactgttaattttggggagaaatgctaccccggacaattatgcgattgtcaagcgaatatcttgattttatccttggaataggtatattacttcggttacaccagatatgaacctatctattacttcatattttactcaggcttggtcgggacctgtgttactttaaaggcattaaaacatacagcgtcgacaatcctcctaatcaatatcctcagaaggaatttactcgcaatagcgaactgagttttttgcctgtacaacggtcgtgcctactcaatcattgccgcatactaatctctatcatattgcctttacggggcgaccaaggaggaatcctatctaatcccagggcacctggaacacctgcggaacatgcttcaataataacatcgtataagtctatgtctgcgcttgtgacgtcatagtacttcttctagtgatatattacgccgttggattgggatcacgtttagaacgacactgtgaacttctatatgtactcttttctcacgatatgccgtcgagttttttatcgataataggcagtgttggagcgggacgtgtcattagtaataagtttttcctatcaatttcctgcgatacttgactcctttggggcaaacatagacgacggttggagtcaaggtgaaccaaaatagaagtacctgggtaaatgcttcataggcacttggacaagacattaagtcgacacactatgccttt

SEQ ID NO: 328 - DNA, 2000 bp, Artificial Sequence
  Exemplary stuffer sequence
aatgttcggtcccgggtaagctatcattctataaaagtcccaccccgcttatttaagattcacagcgccgcaatgacgcggaacagggttgtctatgatgacctaactacggcactttaggtatcatatattgagttgagcgaatggatctgctaggcttcccgtctatcggatgctttaatgcaggttaatggcccgattgaagtttatagtatatatatacactgtgatggtgtaactacgttacttcgttactgatcaattttcaaattatctcatttgttaggctacaactaggactaaagctcaagtaaccgatgcgaagaggccgagatggtataatcaacgggggtgtaatctaatatacgaatcatgctaggagagcagcttatcgtcaaaactctgttggccagattctaattactctttattgtatcttttttcatgtagattaaccgtgaagacagtagttcatgtacgttagtcaattattgagaacattagcttgaatggacgcgtgctcaaataataccccagtaatctaaaccatattgttaatcttttacaagacccaccaatgacctaatgagttcacctccacatacctgtcattaggtgaccttatttccacatttgtattaaatactaataactgaccatattgtgctgtggttctgtacacttgtatacctgttcggctaatactagtcagtgatttcatagcgaatataacatttgacaagactgtagcaacaagtttttggtatagggtttgttaaagcataccgcgcaggacgaccgtctcttacattaatttactcgttttaatctataattatccatataatcaactagtcctgagccaaatcttcaatttcccccgcgtttgagattgcttgatgaggcgaaataagaggcgaacggaactccaaaaaagagcgatcttttatcacgtccctccataacgctttataagtcattagtcggcatcgttacaaattaatgatagaccagaaagtacacagacgtgtcttttatcctgtaacgaccctaattcggcaccgtctactaaatgctttgccgtacgctctgatgattctatccagcgattacgtatatgttccggggtaactacctaaatctaatgcggccataggcccatactgatccgccgatttcgcgcactgctttacttatatacatcagtactactcgggcaaccggtaaataatttacaatagaagtttaagtgcagttacatgcttaagatatcgagagaacttgtgaaatacgtacactaggattttctcaaattcgtgacattacaaggtctggtttcgcgattctcttggactgatataatatgattgaaaaatgtagtagatatgatcctggataacatttttaaacaagtcttgggtgagctcggtaccttaaatccgatcatagaatacaacatggcacctacattcatattaaatagtctattacatgataagactccttcatgtctgaaacattggttagacaattcgcggtttcagtgggtagcgtgttctattgacttcgaaatgagaaagtgtttcggcgcgtacggtatatcttcccccatgattatacataacatccttctaaaaatcgcgccactgcagggtcctcttttcttatatattattgaggatttggaccgatcaaacttaatattaaatatgattctacatacaaaggtaatgatggcaatctacttgcgggctcgactcgtagtctgttcaatgaaaaatacatttctcaagaaataatcttcgagctatttcactctgtagttaaagtttcaatcttgttacatactgcttatacaaatttaatttaaaagcatgtgtcaatttaaggctaaatgctcagtgtaaattgtattggtaaactccctaagactaatgaataacttgataatgtggatagattaaatccgtgcaagcctatcctaaaatcaatttgaagtg

SEQ ID NO: 329 - DNA, 2000 bp, Artificial Sequence
  Exemplary stuffer sequence
tacaaattgtccacgggcgtgaaaacaagcccattcttcttcaattgcaagatttgcgatacttaaaccttactgatttaataatcgattcaaaacgcaagagtcatgaacagaacgagaccccgccatatttaaatgcacattcgtgcagcgatgggtatattgaggctgtgagaggctcaattaaacattttaccaggagatgggcaaaataatgcgtggggatcgcgggactataatctaatcagtcatactctaaagtgagcttcgtgatatcttgaggataaaaaagggcctaagcgcacagggttattgagttccagctaatgatgctcgataataatcggccgtaacttcaatgcgaagagaatatacgattctgaacagttacagataaggcctattaggcgcgaaaatagtcgtctaaaagaggagaactgctggtcgagaatgagtgggggttattctaacaaaggtagctaggtgtggttataaacgagaaggactacacccaattgatctcgataatagggcgggattgtttattgacagtagtgaggtgttctaataacagaaatttagttaaggtgcgtattcttggagtagagcacaaaacccgctaatgagcattgtatgaatccgcgacaaaagagcaaagatcacagcaacgaaagtctaattgaaatagtcctcgattatgccggtgagttgaaaaaagttgtacgttcgtttatgccgttctagataatttacacatcacattcctcacgtaactacatgatttacctactatcacttccaatcaccaactcggatttaggaatactgtaacttatttccgattatccgattgagacctaagcagaaaaacataagatgcccatccgaattgtgatgtggataccagttgtgataattcgtcggattgaactcagcctgcttaccgcttttgatcgcagtcgccgcgggtagatgtagttagcctcaccggctggatacatatctccaggaaatcgcggagtatcaatctctagagtaaatcccctgccttccgttgatcgtcttgctcacctaaatgtctgaactaggctgagaacacaaccatactccggccacgtagacgatgctgaatattacgcagctatactcaaagttaaactcttctcagtgatttatgatgtagcttagtgatctttacagatttggtatcgattgggaatccagtttaaaactgaaacgacatatagaaatatgtaccaatctaccagcgcaaaccgagtcgaagtcatattatacggtaaatcaccatcgtgtgatatattgcaatttgaactgatttttaatccctagcttaaatacttcattgatttctcgcctttaattctctgaacgttacaatttttctgcccaacggtcctcctctagaatacctcgagagccgacacaaatacagttagagaatttttggtgatttgtgcgacttattagaaccacggggtcatgaccttagcccgaataggtagtatccggatatctgaaactccaggcagtaataatacattgccggaacgacaatcggatctagtgaatgcgacatagacggtaatatgttaagcacctcatagatgattactatcaggaaatatcaatttaaagctgcgatgaaagggtcaggacccagccctttcaagtctacgtaactccactagccacattgtctaagggtgccaatcatagatcatgcatcaacaccggcgatacgcttgttcaggcattcatatcttatagttataaaatttgtttatcgtgtgcaggggtcgatttttctcactttcggcaaccaggaaaagtagtaattactatataaaatgaaggcgaatttcggattactctgcaaaaaatcattagaatacacatctaggatccggaggtatctgcctccatgaagttaactccattgtggatatgatgcgagtaacatatttaggtccgaagaaagg

SEQ ID NO: 330 - DNA, 2000 bp, Artificial Sequence
  Exemplary stuffer sequence
atcatctacctaagacagagctgaccgtatccattgtcaatagaacagcaacgattttttccatcgctggaagagtgatgcgcactagttcatttcggacaagtaacttggacgcgatacaagatacaatcgatgtcacagcctctttagtacataccatggaattatgaatcgactaaaaacgcagacgtataattcagctgatcgaatgatttcgattatataccgaagtcagtgacgagaaccttcactttgcgggataccgaactctgtcacaagaaataagtataggttagaatccagagaaaacattgaatattatgttttttcgcaccaaaataatccaacgatgttacgcttagttagtggatatcatgacttcactaaacacttggattgttatctaaagtttttatcttcctggctgcgacattgtttatttaagacgtagttaaaaaagtcgaccacggaggaggaattacatcgtcgctgatgagcccattttcgctaaatgcagtcgactacgaagagtttttcgcgtatcgtcaacataagttgatctttttagataacaaacaaaactcttcgcatcgacgtaaaacatttttcataggcgctttttacaccgaagaatctcagcttcagaattgtacgatgtcttgtcacagatatcctttaaacaaataactaatagcgttgattgtttgacatctactccttattgttatgaatgtataccatattgttatatgctattaaatcccacatattgcggttcgcactaaaatgaacatctatataacttgactgttacttgaattagttatggtccagctaatttttcattctaggcatttaatcctttatgttccatagtttccttcgacgccttgaacgatgggtgcgagtccgacggactaacatttataaacacatttgtgggtttgggtttgctacagatatctggacgcaggatgtttagagtaacatctgttgtcatttggctagcaaaatttgagttacctgatagaccttcctcattcccttaatattaaactgtctttctcgaataccgttcgcacagggtccaggaaatgtgatgttatgacggcgtgcaatggttagtccttatgcaggagtttctccgcacccatcaatgccattattttacagtcaaaaaaacataaacttgtatgacgaatgcagacctttgaacttttgttaacctacttttgtaaaaccagcgaaccctaacagttatgtaacgagatccgttaaccaaaagcggttatccgaggataagcttcctacgacgtcacatttgtcatcttccttaccggtatgaattgtatgcaggtccctattcgaaatgtggttataactgatgggtatcagcaggttatttataacgcgtactttatccttgtaggttagttgctcagtacgcccaaatcaaagaggaggccgaggtgcaggaaggacctgactgacaatcgtaactaaattatccaacaggattgttaattgacaatgtttacactgactatggcaaaaattgtctcccaaacggctgcggacagcgttctttttatcgatctgaggtagcacttgcatatggatatagcaataagaaatagggagataccagcgaagaacggagtagatgcctgtgacgtgtgccgacctgacattgattatcgagcatgcggattaaaattcaacaactattcccgtgaagagtgccagcctgtagtcaattattgtggatattatctaagttcagatcatacctctcgtcggtgaaaacagatagaggccaaagggcaaatctattgaatgattgacaatttgatcatatacgtgtctaagaattaattgtaacggatgcgaattcgttaatcttcctggggtactcttctccacgtcacgagagataacaacaacatcaggcttctgataaatagcgtaacaacgtattatcaaatgcatcctgtctgtat

SEQ ID NO: 331 - DNA, 2000 bp, Artificial Sequence
  Exemplary stuffer sequence
ttaatgacccctgccttactgcataaatctcctaattgtgtaatcactcctcactcagataacgctttacgtatggattaccaagtaagtgaaatcactatacaagagattgcctaattttgctaagttagcgttgttcgtgttttataattttattgtgagtctttcaccgaagtagaaggaagtaaactcgcagtttcttataaccacttctaggcgatgtagacgacatagaaaatggggtaaggaactcataatttttaagtcaatgatacagccttaaaagataaaaattagattaccgtttaatgagggtacgtgaccattaacagtaagaaagcctgcaagcatgggacaggtgctattgcagagctcataaacgaaatgtcgcttgggcgtcctgcaccagatacttagtggcggatgtcaatagcgaggacgaatcattggatgaatattagctagtggatacggaaaaacgtgactacgattgcggcatcgagttcttaaccctctcatggaggcatctctcgaccttacacagtgagagtgcattttgttcgccagtctactatgacacattaaggctcaaacacgctctgcttattcatttggccttggggttctagatcacactacaattgccctttgcaagaaaaacaaatgtcattgaaaaattaactgctgtcttataaacctaaactaccagatactgtaattggttttaggtttgagcatccaccaacaccaatagccaagattgttaaactctaataactgtctaatacacgtgcatattcatagtgaatcagtgcggttcattttctgaagagctccaatctgaacgatacaaggcgtcctgcgcgtggattaaaaacaacttaagcgttacgcagagcagtattccattttataatataccgtttgccgcaggaggttatattgtagaagattagttcattttgtgggggatttacaggccaatatttaccaaattttacgaggtagttgaacctagtgttacttcgtgaggctcgaacggtcttcccgctccaactgtacctttagatgggggcttctttggatgtaacgaagtaccggcttaatatgagacgtttgtacgcgaggcattcttatttaacccatacttaatcaattcaaaatttatcttggtgagtagcactggagaatttggtatccatagcggaccgatagaaagattgttataccaaaattcatgaatgacgcttagtattttctagtttgataacatggttaagactacattctatccgaattcttattaaaattgaaatgacgcattgcatgctgtgattccaaaaccatgccgacaggaggtcttcttaaaaattcagcgtgaggttactacaccttcaaaagtgcataattggtggacaactaaaggataattgggtaagatctttctacattccattaaaaaattctaacaaaccctatctcatgttaagtacttatgttgcctcttactacattgaccctacactcagatatgataaattgatgtttaacctaactatttaaaagctcaataccttcctttttacgcgcaataaaaggttaggcacttttaatgtgaaatttcagcgaaatattcgatcttgatataactaagtttacagttcctattactactcattataatagaatgtatgggctatgaataataaatggacccttagaaggataaatgcattgattcgatgctagagtaaactgatggctcagacagaatcatgcccatggggaaacataacacctaatcagcatcaactaaaagtcacatgtacgagagcagaatcaaatacaaatcaattatataacgtgaacgtagaatccggaccagggacgtttctactctgactatattaccgccagctgctatagtaatcgcgtatggagcatgtatttgctgactaatgctaaagtacaacattactgtgtaatttaaaatgctacct

SEQ ID NO: 332 - DNA, 2000 bp, Artificial Sequence
  Exemplary stuffer sequence
tgtacttgtcttcttgtttgtcacatacggaccctaaatgaccttgtctagttatccgatacaccttgcttaagtagcctcccctagggggaacttattacggaataacagttttacagtattaatcaaactcttatccacgttttcctgtgatcacaacgtattgtttcccttgatttgttgagaatctctattgagccttttatctattagagtctccgtcgcacataatcccggtgcgttgaacagatactggctagactccttacttttctatcagttgaacggaggatacgagcttcaaaataatgatttgtttgtagatgtcagagcatcgtcgtgagaggaacccggatagggggaataacaggtagcgttgcggttgcctgactaaaacccaggactcaagtttcattattaacattatttgcatgaatgacagtgtcgcagatctggtataatgaccaacgatcgtttagtagataaattccaatctaacaaacactaaccagtatctcagcccacattgcatcttgttttagcaatcctgcagatatcagaaccctcctgcagtgaattgactagtgcacgacggtaacatatctctttaatagcgcaccgtcctcaacgtagatgttacgtctggggttatattgggccggaatgtcctgggcttggactaatgaaggcaaaggctataaatgtgcttattatttacttctgcgtacttatttggagaatgtcatattaaagatgtcgcggtggtcggattaattgaataatgtgcgacttggatgcacctcaatcttcattgttttgaaaagtctggagacgtgcaattacactctatatgtctttgtattaatcgttataagctctaaaggagatagcaagctcgggcaaatggtagattaatgcttcaagaaaatacaagcctggggattcacattccgaatatacaactaatgacgctctcattctcttgcaagtatagtaatcggcccgctactctatggggagtatggcatcaggagagagtatcattgacattcgaagtttgcatactgagcaataagcgggtaatgcttcaaaacaaagtgcactcacttaatgtcggacattgtttataagtgttagcgctcaattttccgcaatcacgctcgagcactaatagttggagttcgctttagtttgataataacaaatatgactttgtcgcgagattgcctatttgcatccaggactatcgaacgcaacaaactcgtgaagaggccgcattttaactgcaggatagtaagatctaattatgaaatacatagtccagaaaatcattcgagactacttaacaaatagtttcagaggttctagactttctcaaatgtatgtagttcgtgaatatgtagttatactcaattacgactttgatttttatttaccgcctaagaaacttgattgaaataatctagaagcctcaatcctgctccatcacaaacataatatactgaaagctagagggcgttaccacagtggtacgtctagattccaaagcgtgctaggagattagtggtcgaaacgcaggttccgcgagcagtatcaccctacaaagtagctggttacagtcaacacctagcagcaatttcttcacttttgttacgatacgtccgtggcatgatcgtcgttgcctaattctacgacttaaagataccgaaaaaagcaaaatctagaaccatgatagagctacaaaatccctctacccgttcgtacgtgcttcctaatcagatcaactatgtgagcgacatagttttagctagtacttgagcgggagttttgttctcgtctctgaatatataaagtgtttaatgaagtgctatgagggccactcatctttagcatactaaatcatcagacataaaggtcacccgaaataatcaagcagaagactaacagaacatgctaagagaggtctttcaactacgcacttgatagataaccgttagctc

SEQ ID NO: 333 - DNA, 2000 bp, Artificial Sequence
  Exemplary stuffer sequence
tcacgacgagtgaggtctgagaccgtcatcaaagatcgtaacactttttaccgggctgccataacgtaagatgcatgactgcaagaaagttcacggtggtaatttcaatgagtcattgtcattccctgaaggacgtataatactatgttacgtagattattagggatccttatgcgttgaggagatatcttgccttgagtgaaagaaactcatctgtttagaaacataccaaatatgtcagacacggtcggctttgataagagtccctaactaattggctgcacattacgattcgccgaaaatatatgttgggagtagtgtacacgattttagacaaattcccgagatgatgaccgtgacatgtacaatcgcactaaaaatccccggtattagactttgaagtggttttggtatgtgatcttaagcatattcactatactagcataacaatggtggttgcttttggacgcaagttctgagtatatgactatgaagcggaatcgattaattatgtcttccaataaagcttagaagtatggttcgtgaacagcttccagtataatttagagaggccgacaatatatatagggttttatttactattggccaagaacatcctcagtcgatctaaacttcttccaaagcactaattctatcgcaaaatggtattataacaacactaatcttggagtcaactcatatacgcgcgtgtagagtcatgtaatactcagcggctaactacatgtattatgtcaagtcttccttgctatgaatactggtattcctttgtggattaaaacggtaccgtcatgtaattttgagataaagatctaggacggggaagaaaatagtaatacggtatgtatgcgttgagttgggtctggatattcagtcaactatgggtaactgaggactttgacgctgcatcccctgctggtgcgtagtcctaaaaaaaattctctgggacaatatgtcttcacaagatccttgtgagaatcccgcttccggtccggctgggccatatagactcctattactttcaaacttcgcacagaatcttaaatatgagattgtaaggaaactatcagatctgctctagacaccgacggaggagctcccggaacgttccaaagcttttttttctaagtgttgcacttggccggtcgtacacgcagagcggtagataacccaaatacagttcttctctatgtctacgcccattatgggacgcgtggagtctctgtgacgttgacggtttataggttaagtatgcttacggatgaatattaatgaatcgtcgtagttattgaagacggccgatgtagtatgcaccgtcagccgattccaaactagtatcttgctcctgagttactctgttagattcctgtcagtttatccattttagtgtagaaatatccttgaatggttgtaccatggctcctagaactagacaagataaaatgttataccgtctggtgaacatttaacctcgtacttatccggactaatggtaattgtcgaccgcctcctgaaaactcgcattggtgtcgaaaaaagcaatgagcgcgtatttttatggagataggtgcatgtattagtctgtattcttagatgctctgtcgataacatgatgtaatgcgaattgattagaacaatctgagaggctgaaattgattgcctgcccaaacacgatacggttcgatagctagctgccgatgcgcttcgatattaaacgtaggcaaagacttccattctgttggtggtaatcctatcgattccttaatgaacccacgacattggatattgatatcgtgcttagatatttgccaccatatgatgtatataattaaaatacatatgcttaaggcgatagtatttactccctgtacgcgcagttaccgttggcatgtaacaatttaatggcccaatgaagcgactacgaaccatataatttgctacaatagtactattaacatgctatgaatttatgcaaaaaaaaa

SEQ ID NO: 334 - DNA, 2000 bp, Artificial Sequence
  Exemplary stuffer sequence
gagttgattttccgcatttcatggaaatataatagggtaacgtttagttacggaacgtattcttttgaaaactctacttagtgtcgcaactaaacttctctgttttagtacagtcaggattagagactactaagaaattcctgatctgctcgctactgccacactttacgcaggaggcttgttttcgcagtaaccggtgagttaaggtccaacagggtcagatgtcccttttgtcaccacgaatcactggctcattagaaattgatagatttgttaaaacgaacctctatgtcaacaaatgcttggaacgtcattatgacagtgttttgatgtcagtttatccagaagggcgagagggtcatggcgcggtcaattagaggttcgcatattagtacttaggtattgtcagatcaccggagtttggaaaccctgcttgtgtgatacctacaacttaacttggcccaacatgagaacgttccatgcttctggtatccgtgtttaagctctcagtggagaaattcttaaaatgatattcgtaactaaaggcatgaaacaaaatgtgaggatcggttataatggacacagtcctgaccccttcgattgacctaaaatattgaaactacattcaagtagcgagaattttttaattgttcctaaagttttattattagataagtggtcgatgtgtaggaaataagagatgataagaaaaccagacgttatttaaagggaaatgtccaccagtgccccagcgttataacatgatagccaagaatttggttatacgcaaagttcgattgcgtgctcggttactggagatcaaattaatggagcttcaataatagtactaaatcatgttttcaatttcttagcacatccccactaatagtttgtctcagatattatatgatatagttgatcgaccctgttatacgcctaaaaccaattctctttcgctacccgagagtgaaaacatattcaaagttgtcagcctcgacgtttaatcttcgtaataatttgtcggtaacagattaaatacggaagacaaatattattatcttcaactgtccaaattctccgtctccatttgagacttactcatacttcagtgaccttggcactatagctgatgtttggagagaattaaaccgagatacttataataatgagagctaatgaaatggtagttcgtatatgcggttatagactgtaagaactatccaacagactctgccgcactctcagatttcatcttaggctaggttataatgtatgggacggctcggatattctattgaatttaacaatttcgtccaacaacccttggtaactgagtttcccgattacatgacgatccagcttaccgtaaccatagaacttggcaatcctctccttaaggcgcatgactagatcatcaatcgcacttcttcaatcaagttctctatctggcgcggacatactgttttacgtctcgtttcattgtaaaaacccttctgtgtaataagaacacgcgactttgatggttgcgatccctacgtaacgtgcacttaactacatatacttggtgagattgtgctccatattgaaagtcgatgttaatcaagacggagttgtgattaataaaatggcataatacacctgtgtttttcctatataatccagagaggaaaataactgttttccgaccaagtttgtactagatttatgattttccgaatatgcatctgcgtgagtgtgtacgtctgtgtgcatacgtcattcagaaagatcttccgtatgtgagaccttttggatcagttgttcatttttgtacctgcctactttagaccaggttctaaaaggctcatttaacacatgattattatagatcatataaccattactcctaatcaaatttgtgccatcgttgcaaccgaaatcgtctagcaagatgatcatcgagcaataccgaccctttatataggctcaaccctatattcagaggaaaatcacggtttgtc

SEQ ID NO: 335 - DNA, 2000 bp, Artificial Sequence
  Exemplary stuffer sequence
gtccatcattgactctgttttctcgaggaactctgcaaaccagataagagattattagcatatatgtacctagaaggacatattatcgtggacatcccgggtgtttgctatttgagatttattgattgttttttggtaaaagatctgatttacatggcattatagccgaggctcatgtttacattagcatagtaggctggactagttgcgagagattttgttacccgggatcaattgccattacatcaaatcacgtgaaacgcttttccaatacatgcatatcccagccgatacttagtacgagatgatagttgtacgacggatatataattacgtctatacgttataaattgtcacctgtcaccactttctgaattaaaagctgagggacgagccgtattaatactaagagcgtaagagcctcctagggttatataacttccgcactcagctattattattgaacctgcgtacaagtatctacttattcaagttactacgtatgaattagtaagcatcttgttttacttatgaccgcaatttcatacgttgcatgataagacaagttcaagcacaataactacggcagtaggaattgtggctcgacaagagagagctgttttcgccgttctggggatgagcatatttaaagttgtttaacacatcctttaacgataacaaaagacatacacaggatgaggtatttctgtcaagagaattggtagtttgtgttaagaagatccctgaccgtccttagatggaagaattaacgtccatagctggaggtgttgtctttattcacggaagcataagagactcgtagtacagaataagacggtctcagggtatccaccaggatcaacgccagaaagtgggcaacagatcggaagtggaattcggaacaaacttcatatgtgaaagaaaagctttgatacgacttccatgccttggtgataggtcaaatttagctattagaaactgcaatgggagatgttcgtgcatgggaagtaaatgtatcgaccataatcgctctgcgggctagagcttgcggacagttagcggttctttagacgggctgaaccctatcgagaaccgatacagcaatgtagtccattacgacatatgtgcttcctcgactttactggagaaccttaagacgcgatggattatttaactaaatttccagttatctgaactggcataatttacaacaaacctaaacattttccatagaaactcgttatgagcatttcatgcagtgcgtccactgtgatatctgtaatggtaatcggtcctcatgcgatacggctcggtagtttgtcttgcgacttaaggcaatgatgtgtggcatgctgtccagaagcagatagatcagggtcaagtattgcccgcccatttaattactaaagagaataatgcacataataatctctattgttaatgatataattattctagtgatttatatctttataaggtaagcgatttcaacaaattaaattaaacgccataaatttctagcaatttagatactgtatgggactattagggactccataattaacgtatgacatactacactaataactaaactctatttgacagttgcattgcttaaacacccttgtgtgttaaaccatacaaccttatgtctggctatatttgtacttcaggaccgggattcatgataagtgcttaggaacctagacgatgaatcaagatcaacgtcttatttataaaacgttgacacaatattaatcctacaagatctaactttaccattaaacagaacttgctaatccctaatgaccaacagacttctggcaacgagaaaaaaataatcataatttgtgcggtacactttagcattaatttctaggattcagctagctgggcctagggaacacgagctttacgtggcgtcgtccgaatcgttagagaaacattgtgagatactcgatatttttatcggtagaatcctccctcattcttacaatgta

SEQ ID NO: 336 - DNA, 2000 bp, Artificial Sequence
  Exemplary stuffer sequence
ctcaacagcattctatagccactaatcttatctcacaggcgcattgctgccataccgttagagggtttatgagtgtggtgccaaatttaatttccagctattgctgagaagtcatataagtttaagtgcctctattcatgaatctacgaagactacgccgtctgcgcactggctttgccgtcccacttaatttaacgttaatatgcaggtccgggttaattcatgaaatttatacgagggggtagattgtcgcattatacgctcacctacaaatctgcctatcagcacagccattatgactagatttaccggggaattttcatatacacaaaccacactcattttcccacttataggattgagtctcagatcacacttgtgctgcttgctgcaaatccttttatcattgttcatggttacttgtttaactaatatcattcatttaagatagggtatctttataccttgaggccaagttttttcacagaatactgaacatcgaaacctttacttcaaatagatcaggtaagattgtttttcatttaaagcgattcgctcatacagctttctgttaatagtgatatggattggaaactaaattaccgagatatatcgtcatcgtcggcaagcagctgctttatactaggatacagaagacggccgtttccagtaaaaaaaccgccgattcgatcttcgattattacctttttacttgcggcaccaaatgtagctgaattatgttatgagctatgcgtagtataccccctttgtcctagtgctaggctctatcattttatgaaatttaactcttgctccaggatacgtcggatgtacttttaacaaaatctactgagaggacaggattgaccacgtaatagtagaactgataggcgggatgataggatcatgggcagtattgctgattttagaccttggagatagctgcttaatgagctcctcgacctcacacttactgcaaggtcaagataagaaaatctcctaaagatcaaaccattccaaattcgtgtttacataaattttactattatacatcgtaatgttaagtgatttagctactgtgtgtctaggatccaggatagtcgtctaagaagccgaccaacgtgctaaataggatttgaacagcgttatagtttagtttataaggttgtctattttatcagttactgcacgacacatatactctcagagaatagggtatcacggtatacatcgctatcatattgactaacgattgttcacggcttatattttcacgagcattccaatgtggtaaccattcgcaatcatctgggctctcagttgttaatgtagaatttaaccaggttccgtattagtcgaaatcgatgctctatgacctcaaccttcctcttgtcatgatagggtgactaaagaagtttccgatacgcgacgtgaagtccgattattatccagatggtaaagtgaagcttaaaacataagagatcattctctctgatgagacataatgatatcatttcaaagttctgttaataatacaactgctagtcaacggaatcctttccatctaaaggcgaacactaactaatttgaatgagaaagataacactaaaaccgccaacctagtagttacttgagctaacacatatattacttaagtagctttatctctggtctaagtcggaggtcacaatgacttggacttcttttagtttttcgagtacaactagacaatgacctcccgacgtagcatatagaaagttagaacataggattaccgagtggtaatagcccaatcaaattatggtgcgaaaagatagtactgtactcattacttccggtatgggacaaagccgatctatttgtcggagcacgttaattttatgaccggctaccctacgtttactgagtctaaaaatttgtaaatacaaaaatttttcccgcgctaagttaaccataactctcaagttatacggggtaatggatcttaagttcccggaaaa

SEQ ID NO: 337 - DNA, 2000 bp, Artificial Sequence
  Exemplary stuffer sequence
gtaagactgattaagaaattacatagggacctggaaccggtatcagatttcaaattttggataataaaccgccaggtgttaacccatcaacatctagtattggcgtagtgagatctcttgcatttcagacatcctgggacggcaggagtttctatccattttccgcaagtgttatgctccaattgacagatatgtcgccgaggaacaccaatctggagaatatttagtcgagaggcacaactggtgttataatcttagtgttatcaagatgaccttttggagtcctttggatacatgaacccatacaaattatcagcgctctactcttctgtaacacctcggaaatacactgaaacagatgtcagagataaccatgagtggtgattgcaatcggtgaccatgttcgtagatcagtcctacgagcgtccatatggcgacgagggaactccacctttcgagcaatcatattggattgagcaaatggtcattcaaaaatatactgttcactctgccaatataaaaatagcactcgttttttctattaggacgatactaagtgggcactttatccctaaataactttcacaaacccgattatagatcccccgtatccaactggtagaaggcggctcggatctatcaagcatttgccgaattttgcgtgaaatttttccactgactgctaagcataaaccgatgaagccaatcttgaatgggttatcttgaaaatattttgctagatttcatagaaactttgattaactatatacgatatacttatgaataacgcgaattacatatatagacatgttctacgttccctgaccttgcgtcaacaaaaatcggttatgtcttaatcagaattgtattataatacatacgtagccgttttttaactactgcttataagagaatatttctatacttactacacagatgtttggactataaatagaatgacatgggggcaggggaatatgtataaatgcctgtgtgatctccaactgcgcattttgccgatgatatgtagataatactttgagtcttggacggccaacgcgcacagactacacactactatagacaatggatgatttcagacgcaataaaatgctaaaatcctaccgattgtcatatttttaagtctatacctcaccgtatattgaattcatgtcgtatccgagcgattttcgatttgccctgagaccatagataaaactcactgagctctaacgtaagattcaattcaatcaattataagagcaaaagtgtaacccgtcgaagttattaagctgaaatagtcgcaaaaactgtcaggtattgctgtccaagttagcggggcgccatgagaatgtgaatgacacggctccttgatatcacagcgtcaatgtttaggtggattagagcagagatataacgaatgctcatccgatatgacgtataaacaaatgagtaatgttaacacttttatactccggtacctcagtattccagatctgacgtccgtggacacagtcctcaattacgctgttattgtatggactacccatcgctgcttgacacgatcttgaatttatatagctacgaatgcagaggttttgcaccgcttggcactaccgagtataaggattatgtcagtcgaggcctgaagcggggactgtgaaaagcactccacacacaacagccaatgtagagccttcgtgtttgaaattctaggttttcaacatagttttttggctgctattctattaactactagctttacttgtaatcttcggctaaagtaggaatgtattaattcgctcaccgaatatcgcccatccttgaccacgatgtcccgtcaatttgtaaaaggcatctagtattcatcacggtatggtatcccttaagttgtgtatggctacaaaaaagtaatggaatctaactaattccatcatgcgcgattcatgagctcgtgtctgtatgaaagaatataccattcaatagacacaacaatgatt

SEQ ID NO: 338 - DNA, 2000 bp, Artificial Sequence
  Exemplary stuffer sequence
caagctagtctaaactaacaacagcaggagggcgagaacgttggccacaagacattaggcgttctgtttatcaagcatcgacgtctaataattttaatactaaaattcgtcactatctagttgttcaccatggatttttatgtaggcgatatcaattcagtaaggtaaccctagttctctgggctcatgtatgaaatcgggaagaaagatatgaatgaaaagaacctaactactgaagggtagtcgacgagaggcagctaataggcaacctttgtcccttcggacggactggttgctgaaattaatttacataaattaatgaaacatccccaacgccaccttacccatagggcgtctcacgctatacggtctattttaatgcctaagaatttacgatgagcctataaataccttagttgtgaacgaaacgcagcacacgacaatcgtacaacctcacttttaatgttatatacgggcgcggcttggtaaatgccgtagctctagtaacataatgcatcctcaccataccagcaaagctaaaaatcttcaaatattcgtataaaactaaccagtttaacgtgtatgaggcggtctttttaccagtttgggagcatattgcacgtactatcttctttttagcagacctgggatctgagaacttcccctgggtagtcttacgattatagttagcctaatagattatttgttcgttaggaagaattcatatatactaggttatccttcaggttgaaaattaaggacgttacagatttttcacaattataccgactaccataagtgggagcgcgaatagcatttgagtatttggatcaagcatctgctgggttacacgtattaattagacccttgccgagatctagggaaacaaaatccagacccgcagtacgtgggtggtatgacgcttcttaggataggagcgcaagtccatagacctttatattactacgtttacctgatctaaataatctgatagaaaattaaccaggagtcccattaaggtattcaaccacggaacagagtataatctggttgataaagtcgttttgatctgttaaagatttgttaaactaaacgagacttctttgggtaacatcatacaagtctgataaaggatgatgcagggactagtctaaaatgagggagtctttgggtatccaccaaataatttcaggagttaagagcacttccaacgatgcagtcctttggccttctcgtgcgacaaggcaagaaaagtttataactctacagcttgtgtaactcgaaagctgacctactatataatgttattggaaatcaaactcagggttatcttcaaacagtttgttattggctagacagctattacctttaattggtccttaatcttgcctatggacatgctccacacattaaacatacttaatggcatgcaattatagattgtcccgttcattcactatagcttcataatggttggggtagtacacgcaaagtctacttatatgggcaacgcgccggcccgtctttcctgttaagttacgggaggtcgctaattactattttactgggaatgcgcaatcaaatcttgattgagaccaacgccaggcccgaactattcttattgttccagagtctttacttgaatgcatagtatcgggatggggtgatgccggccaccggatcaccatggatatacgtcagttggcccacgtgttaattaatgtcatattgttatgggctaatacattactgtattgtttaaatacaattcgtcatgcattatcagtactgtgtaatttatataagcgttcatcattgaacgtgtattttgttggtgcgtactgagttagatattggagaaattccctaaccaaggaacaatgactggacttgttagcgatgtaagagtaatgcaaaagttaatgagactgatattggaaacagtattgtttaggctagtctagaaataaactgctcataaagaatcttgcagttaatat

SEQ ID NO: 339 - DNA, 2000 bp, Artificial Sequence
  Exemplary stuffer sequence
ttcactattaagtacacctagtcagacgtgaaagttagttcttttcacgtctcatatagtgctattttcgaccacgtcttgcaatcgtgatagacagagctgtcattaacaagatcaagttataaaattgtacgggttgtacctgcttatagttatatgttgaaattgcaaggccgcgttgtgaccggtttgacggaatctgaagggattagaggagtttatatttaatttctttcatgtagagatagaacccaataacctctcgctacatagaactaacgttttcgcagtgatttaccttgtgaagtgcacagtacacttcactgccttttactcgcatattgatacagtagccaaaagtatcattattagtgcataaccttcacctattccaacggttttacgcattctgcgtacgttcgattgaaatagaacaaatataactataattggtacccatgatgtaacattttacctcagtaatatgtcgaagataggctaagtccccagctagcgtaactagctaagccttgatgcgtattccttaatcttgtttaacgtctctgcttacgctagtttttagtagagcataagatagcaatttcaggatggaacgagttatagaacagaccactcctacagtgagtagggtcacatgtattgtccgacactgtttattcaattccaatcttttaagtgcgaatataataagaagcaccctttcaaacaattgttataatacgttttcatgacaccaacgatgtcgactatgatgtgcttctcttttggttagacatctttgcatttcgacgactccttttcattgagcaggttttagttagctaagtgtttcctacattgtagcgcattagtctaatagagagtgagcattagtcacaatatagtccaatggatctgagaagccttatgaggcgtgcttagggaacaattgcagtttaggcagaaagagttaccctttaagggtggtattcttatctcatatctatcttattggtgcaaagtttgtctttgaacgacagagtaactccattcgcagccttgctaaaagtggagagacgcaaaagtggaggcacaggtcgtttcttttagtcgtatatccagtttatgagcttcacatttaagatcaaatcccttctcgaaataaaaaggattcccactttaaataggcgattgattgtgcgcactatttattcgtaatctatacgtaaagaaactgaacgccacagcctaatacatgctagtatttcatacatgtgagccgaagacacgcacttcctttttgatgcgagaatttagggcgaccaagtctggtaacattctgtcctagttgccgagtaacatagatataagccttagcagggcgcggctataccttggtagtaagacgggtgtttgagtaatattagtagcttaattaacagcggtcaatcgccaaacggaattgtaactggaatgtcgtataatcccatttatatctcagcacataaatcaaaatggctgtgagatttaaagaggttagtaattgttcagaaatccgaaatcctcataaccaaataaaattcgcatatgcatacttgatcggcggagcgatgaaagaattacacttttagtatccaattataaacatcatttgcggcctacttttcccagtaaatcaatacgtggagaactggctcgtactctgctctacacttattgaatgagttagccaatgtagagctggatactaagctctagaagttactccagaacaattaccacgttaataacttctattattcagagtcgtaacagccctcaagtcctctcttgttcgcctgtcagcaatctcctacggacctaccctgccaggtagttgctgtctaagccactattagagttgctagatttgttaattataatgcttcgccatagtcatccacggtcagggcggtacctcgcagcttgtgtaagggatccctcgagtaactcttgatgat

SEQ ID NO: 340 - DNA, 2000 bp, Artificial Sequence
  Exemplary stuffer sequence
cgtagtattttgtgagctagatggagtactccgattcaaggtattatgaacgatagataccgtggctatatcataggattgctacactgtaggttccagaccttagcgaagcggataccttccgttcggttatctgttaaaaactttacatcttcatgataaagtgtgcctacctttgtatcactgatgtacttccctacaatagatactctttaagacctgagtacgccgaaagaatctgttcgatctagcaacgacaaaacagttatcagcatatccgtatattgtggtgtagcgtcttcgtgtactaatttagatttctgcatctgtctagttacgtgtagggcctatgacggtcccttgcttttcccgggaaatatcaattgcagttgtgaaaattgtttataggaaaacacaaatctaaataaattactccaaggatcttctcccagatgactattcttagataatgagaaagggagactcgattaagtaatattgtcgagcaccacaatctgcctatattctaacttagtaataattaattaattatgagtcaaccaaagggtcgtttagctgattcatatacatactatatttgatcaccacctacgagcagttggcataatttccttgttgactagttttgacccacgtgattcccctaaattttttgtgctctatgaccgacaaccacagtgtaatgtctcaggtaaaaatgagtacatactacttttccagattgcataagttatagacttcggtattttccaaatattattgcattgtactacaaaactaacgggtatgagtagacacaaacgatcacgggtttcacttatgaataacgttgtaacgataagtgcgcctcgcctgcaccgcatcactaacgcctttttcgaggtaataccacgttccgaagaatctatttagttcctcgaataaaacattattgataagtagtgaatcaccagcctcccaaaaataccagaagagagaaacaggtctttcaattgctggtactatttgatatcctttacacgttttctattctccagtgtaagtctcgttatgcaagtttgtcaatatcagaacaatatgatatacaacacctcgcaagctgctagcagttagatgcgatccgatgatgatcgataaaaacttatgtactggacctgctggtttagcctttaagaataagttgattcttgacatacagctcgggcgataggattgaagagtaaaagcgatgtaaaccaggtctgtgttcgatgcagagcaagttcctgcatcggatttttcggatatgcagcttagatggttactcaaatccaattccgggctgttgtctgtacaatttgggaggttgacattgccacctgggcaaatgttgtccgagaattcgcccgatgagagaagggacttggtggagtcacaagaataggcgatttcgccccaaatttaatatccaaaagaaggcgttctactaaccgtaacgttagacatattcgtacagtgaagttcgcactatgtgtgcattactcaagtatctgttgtataggataccttagtggttcagtattaaacacgattcttttatcttgtatgttgtaatagcgatcgttacttatcaacagagttaaaccatggtacaagtgcacaagtcattaagcatctagactgcactacatcgcttctatattcaccatatgacgttacaatctcccaaagtaagtatgtgacaacttctccggccagctacatccggtagaattgtgttaactaacagtgtaattatactccatcatacgatttaaccggttgaatgactaaaacttaagtagttctcgcatgggtctccgcctcactggtaatatgtgaccgctctattgaattcgagaccaggatcaattacatcctcaccgggtaaagagtagatcaggatttttaagtgagtaacctggcgatgaatacaaggttgtactgcagttttaccctga

SEQ ID NO: 341 - DNA, 2000 bp, Artificial Sequence
  Exemplary stuffer sequence
gatttaaatggtaattaaaatcgaaggttttaaaaggtgagaatttttttataaaatgcaatctgttacgcccctaatattcggtttcatgatttgcttaatattgtatcaacacaagcatattgttaaacagtctctgtactttcttgatgaccaataatgaacagatgaagtcttcatatattgaacttcaattgaatgcgtgcatgccattattcgtcatcgagaattaggaagaaaacaattgcagccttctagcgccaattgcgattagtaagcttcgccctcacgtactaaattatattagactgatcggagacattaacaagctgcttattccgtcttgaagaccgtatttcttactgttacggtgtccttaggcgtcatatatcaactaatataaaccggtactttattcataatagccgatattcagtgattgtttgccataggctactttctttcccaaatccccggtatcgctatcctatgatttctgcgtcaggggttaattacggcgacaccagcctaacccaagatcagactaggataatatttcactggcaatactcatcgattaattcaactagtatctattttttcacactccgcaaaaaagggcaaaacaaagtcgtcaagccgggaataagggttattcttgcagtcttcgtaataaaatttgaactcagttattgcgaatttactcgtataaagcttctattatcattctctgattactcaaaaacgctccatgagggtagtagcacataagtagaattgctcatagtggcttctttctctcaatccctttgatactcatttttatattacttacatgtaacgattgttgaaggccagcaaaccatataagtggacagaacagggaacaagagaaaataatacagaaagtagtaactagtcaagaaagtctagatgaatctataagttgtacctatcgaactatgatcgtagcattttcagtctacttgagggagaggctgtaaggaattttagcggccagatatatatcgctggaaccaagttatcgcatggaaacttgatcacgtacagaatgtgatgtacgcgcaaattagatctgaaatccctctgtcctcattttttaattaatacaattaatatcaaaggccttcttttctgaatgttattagacggaacacggaactgcgattcatcatcctaactacacaacacgaactgaccagatttgcgtgtaatcgtcacgtgccgttgcttactctagtaaaccccggcgcaagggcgaattgtgaaaaaatgagtcaattcgctacagtggcaaaaaacgagctcctggacgacacaacctcgtatagcaaggcgtagctcaatgcgccagatattcaggtattgtagcccatgacaacaagaaataaagctatagtaggcatcattatcgtttcgtccggcagcttttttctgacttccacctcattgcgtcttatgtcattactgcgtagggtcacctatatgagtcttcatccctgggacactgaagggagtacgccagtatttcatctatgaataaacctcgattactcctttatgagaacaatacttacactcgacggggtcttgtggtagtgatcttaagattatctaccatttgttcacccttgaaaaaagagacttacctctcgacttttttctatactgggccccgaccgctgacatgcagaatattgaggagatgcagattgatatttacaaaaattaaagcagatactcaacgcatattctatgaaaatcagggacacccagggtggtgctttaggatgatttacatgaaactttaaaaggaccgggataaactggccgccggtctttcactgccacagggatcttattcattcggatatattattgccactcaagataaattctgttagtaagtgttaaagtgtatcattattgcccattcttcagactcgagaacttcgaaggcaaatgctggacgtgtgtac

SEQ ID NO: 342 - DNA, 2000 bp, Artificial Sequence
  Exemplary stuffer sequence
agatccacggccctgaaatcgccatcgctgttcttctttgatgaataatgcaagggctgagttcatcagtgtattcgaatgctactatatttcagtattgtgagtatcacagctgtaatcttcggaaatacaaggatgtttgtcgacctcgctaacactagattattttggcccgttactatttatatttttatgacttcaaaatgcgcttcaagattgtaactctggttgatataggatgcagggaccggctcagggccgctctgcactacattaatacctcagggatctctatttcgttagagcacacgacttagtgactagaatagctttaaatgtaaaacttcatcatatattcctcctggctaagccttaatttcattcttggggctgttgccaagactgctcaagagttagtttttctttctccttgtagtacccgttctcctaagtgcaaataatctatacacacttcatattgggtataccattcttggtttattgtcacctgttatgtattttgcatcaaaataatcatcgatgtatacgttaacccaggagacaatcgaccggctaattccgggaacgtagatgtatgtaaagtaacatgtatttcaatttcttctgaagtatgagatttcagttgcacaaaaggtactcagcatgtcttatcatccatagggccgcaattatagaggatcttgagtggagggtccatacgaggccttaggaagccggcttatctcagcgaaggttatcgagatgctaaatttacggataaagatccgttactcttctttagaactaccgttccaactcgaacatagaatcggctccgaattcttgggtaccttgcagaactgaaaaatagatatctcggtatctaaaggcagaaatagttttcgctctggattggtttctaaagtgaatctcaagttctaggtaagcattcaagtccattggggaccattaggggttaatacgcactgacgtcggtctttcgattgataaatacttaacctcgttagcagtgagggtcaacaatcattaatctccagctatagagcgggttagccagattttatatcggcgtcattccttttatctttgaaatttaggccaaaaagaagggaactggttctattcgcgaattgaaccgcatttatggtaatagatctgaccacgtgctactgctcacttacaatagctagttttcggctcaaactttgtataaggctcactaggcatataacgagttaaaacttttcacatgatacgtgactagcttcgcccgacatactatatataaggtctaccgttgcgggaaaagatgaagatgatattatcaagtctttgactaataaattaacttatgcttacaaatttccaaaatagatattccagtcgtctatccttctattacagagaaaggcagacttaatccgttcattatataatttatttagatgttagtctttctggtgggtcgattgttagtctttacatagaactcctttaatgttcataagtttccatcagtagaaagtgagcttatgggttattcacctttgatattaaaagatttactactgctataatctacctagctcagctgagaggcaagaggatcacatgttattgttataatgctttgattggtaaactatagtgtcaaggcaattcgagtgtcgccaagttacgtcgattagatcgatcattaaaatctaataatgtttagagtttgttagagtaatggtgttgatcggcacataagagtcagaacgcgggagtattgatattttgccgaattgcaaatttatcaacatcggttctacgtatcgttgatgtcctaaggccttagttacgtagcttacatttaatgcgcatagggttgaagcgtgtgttaatcgctctttcaaataagtgttaggaaatatacgaagtaacgaatatcagcctaattccagcgactaaaatgaaacaagagcatccggtggtag

SEQ ID NO: 343 - DNA, 2000 bp, Artificial Sequence
  Exemplary stuffer sequence
ttgatagtgtgattaattagctggtcattatcggtatcgttgacaacagtaggatgatggcgattgtctgcagatttcgtccattaatataagtaatacttgttatgatgtccaacttagatatattggagttttattgctctatttcctgtacccttgtgacgagtaactgctccgtgatataggcaagttaagtgtgtcgcaatatggcagtaggctgaataccacacatactgtctttctaaataacactaggcgactacctttaacttcatctaaggacgttatttcacactaagcactccgtcccgagaacagggtctattgaggctactgattgcgtaaagtagttggacacgcatgggttctagatcctcatctctggtttctcaacatattgagttatactttctgttagttgttaagccgggcgatcaaagcatttctacttcagaaatggaggactgtagttatatactacattctgaagcggtaccattaatgctttccgcattgatgaatatctatatttacagtttggtgaacacaattaggagagtcggactgcgcaaacagaatatttagttacttatagttaatatacacctatacacggtagaaggtcagttcatatagacttctgggtgtgtacttcatcagaagtctcctgtctgtttagccaatcgccaccttctcagtcccgtgggagtaccactcgaatagatcgttgttttcgttgttgataaacggaccccgtcttattttcgttaccatttaatacgatatcatataattgaaatattaggaaacggcatttcaaatacgaacgatttgaacttcacctaccttttgacatttatattacaattttatagggcaaaacgtaatgcacctaaatttactgcacttcagatctaccaattgatttgtcacaccagctatttaacgaacaatatgactaaatattagctggtatgcaatctgaaaagtcaacatggtatttctgcttacaccggtagggttaatggaagttctgcgcccattcgaattttagaactgaacaataattcatgaaaatttacgttagcagtacctttttgtcttactagttgttgcagaaatttaaacattacttggtagcctgctgtgtatataaaagagcgatctccgataagttgttaatctgttgctacctaagcgcttactgtgtgccttggctcgcgtatatgcccaggtcaacatttatttgtcgctcgactcgaaataatctatatcataagatgggaacgagtatgctccatgagggagccggactaggcattcaattttgtttgagtctttagtaaccatacctattcatgcgtagttaacttcgtagtaaagcagcgtttatacataaacaccaaaaaatgtcctaggggcataccaagaatctaagaaacagcgcagtagttcgttcggtttggcaaccatacgaaagtatcattgcacacgacgcatacagcatcctaggagtttactatgtcttcgtttttttgtaggccccacacacattaaattcgatttattacactcagagtacctgtccgccaattcacgtgagtaccttcgcgcagcagataatacattgctatgcgttcagaccattgtaagaaaacagatcatgactctagaaaaagtggccttagatcaataaatgttaaatccggttctctctaacctcgccgtacacagttaaaatcaacgcgcatacataaacattgatcttatgggggctcacatagtgagacaatagtagtacccagtgttatacctaatctaatatataggctaaaaggtagattaattgtctgatcatagatctcaaccgatcatggatagctgggaatacgttataaaggtaggtctacgacccgcgaaatctcgaggaaccacaacagaaaccattgtctgtacgagcgacagcgtatgtactccgtggctggtctacctcggtaatg

SEQ ID NO: 344 - DNA, 2000 bp, Artificial Sequence
  Exemplary stuffer sequence
gggtagttttttctccaaggatccccttaactagggtgaagattgggattaaacctaagataaagatataacggtcactggcgacaagcttacaaatttgcgctttacaacagaccaaggcgaaagtaatcttggccctactaaaccaagggaaatcagtagtagtgttctccaaataggcaaggctaatatctatactgtccctgcatgatgtgttaagccataggcgtgtaatgttattccttttcctaaccagcttttaatgtatccttgtgtaggaagaactgcgaagttatgttactccgaagccaaccaacatgtgtcctcttggcaccatgattcgaaggtgatattataagttattcgaccgtgaagattacatattactggatggtgtataaatagaccatacgttcattgaagcgtgactgaagccgacaacggcttacgtaatgattcaaaatcggtaataaggataacggttatatatagtagaattcgagatggaaaaaccaacttgctaatgacaatattaagggtatatcacactgtggtttgtaaagtagtcacctattcgtgatgccgtgtacttcaacttatagtaaaaagtattgttttctaaccagcggtaacctgttgcaaaaaaccacgtttaaccgattgatagcttgtggtaaagtggcatagagtatacttcctccatctgtagtacttaataggtgttccagttgcagtataaacctttcttcgagtatcatcactaagaccattagacataggatatatacaataagagctggaacttgaatcttctaatgacagactttactaattatagttcaagcgcagtttaactataaatacaattgtcaattcatcatatggtaggcaagattcctttagcctggcgtacagtggcccggaggccttgaccaaaacatggttctgttatatcacgagatggattgactatgctcgtgaatctggagaggcactaacttggtaacgcccgtactctaccgcagcgggacaggtgatagactgtctatgtaaatcgtcatcaatctatatttcaatacaactataaatccagacaagtatccttgagataatagttaatctatcctaactaataagaagaaaagagacgatacggtagtagattaagctttcgcggaaacaagaggaatctacagaaaacaccctaaataagctattccatgccgcctttgctatgaacgaagtacggaagcatgatgcttatcaacgtcaggaacctagctcaaatcaaggtcttaccagtgacgataacatgggtgcggatggttatttgtggagaggcgtaatacaatgtacttgttttcaggatatcaatttaatttcacttagaatacgagacggccgacaactttaacgaatacatttgcatcccacattaatacctgagtgccgctcatatcgtcctagcacaatttttaacagaagttttggtggtgagtagaacaacaacatgtagtcatcttaagcgtatgaaatctggctctcaaattcatgtttaatagtgtttaatcttttatgtataaatcgtttttatggtttagacgaagcactcaaaaatatagactgatgcctatgacctgtgctatctttattttccagggcaaagatgatctttccgagtccatatcttgaatgacttcccgcctgaaccaatacctggtcggaaggaggactcattaataaacatgcataaatggcagatctgaactggacggctgacttatctcacaatgtgttctaaagtccacaccgtttctgtaccaatgaaaggacgaattatacatgcattggtttggttaaaaccaatacttggtaacgatctggaccgggcggttagaatgatgaattaatgcgccgtatgtggaatgaagtcctgttaaaatgcaaaaggtggctcttcgagagttgttgggttgaatgagagaaacgccaccttcaca

SEQ ID NO: 345 - DNA, 2000 bp, Artificial Sequence
  Exemplary stuffer sequence
tagtatctagtttcaggtgtgcacagaatagttatcctcctttgtctgtggctatttggagaacgtattagaggaagcatatggcaaaatggcctgtacacgatagatggtatcatgtttggaggacgctaggcatttcgccctaaacaccgcaacgatacctaaagagctcgtcaatgggcttgccgattaaatacgcaagttttagtcagtccagaccacatttaccggtaattatgcacagacaagatattatgctggtttatagcccatatttgtctccccctaaagtgagctctgatatttggttaggtcgagtagtacagtttgctatctatggatacgatgtaattgtgcttgagatacgtgcatcacgaacattgctaagcggattcgcaatgttcgtgatgcatggagtagtctaagcaatccaacaagcgcctgaatataattttgtcacaagtaaaccttcatattgtctaacatacagagctgttttaccccctcatgatctaaatctttcgcttcttcccaaactgcacgccctattcgcctgttagcgcattcaaccctaatacagctgttgtggggatactctgattgaaacaaagttctctatggaagcttcatcattaggccatacgaaatagaatcccctgttgtccaggtgcttctcgactgcgttgcggttcttattttggctttgctaataggaacttctctcttcgagctcggtcgaacgccagttcgtcaactataccgccttctttttgcgcaaggtcatcgaaactgaggtccatcctgggacaagagatcagttaagcctacacttgtgtgagactccgcagaaaatcgggaccaaagcgttagggcttcccaattatgaggatctatggtgtcattgaaattgataatccttatagggccatttttatccctgacctgaattctatttggtgaataaagtattggtcgcctttcgagggatactactatgttatggacctaatggatgaccatctggaacattagcaacagcaactctaatcttattttatcatcttcagtgtaatatatcgtacattttaggctttcctttatgttaaattgttattatgaaagaggtgtattataagctagttaagcgcgttaaaacacaagtggtctgctgtcattcatataccaaagaaggtcttgatggacaatgtcttcacaagaccatgcatagattctaaatcgatatgacacctaacaaatgcgggctaatattcgatttctgactcccacactgtgagcacgtttattgcggagacttttaagcgagatactcttactccccattgccatatatgtaaaatggacttccaattctgcatatttcagtacatccggactgcgttataagcattgtcgtggatgcatcaccatcccatagttccacttcttttttttagttcagatccaaactacactatagggtgacttattgtcgatcaaaattattatatgtaagtaatagatcatacatcaacaccgaggtctttgtccaatagaaatagtatgtcctggagttttatcaaatacctgccatgtgcaagttcacagaataggacgcttctacagaattcataaaatcccacatccttagcgtaagttgtcagatgaattaattatatttttgatacggccccagttattctcgaagtccactcttaaaaaaagttattgtacgaacttgcataaatcgataacctgttaccaacatgccccggcataaatcaacaacgtggttcggatacgacaatatcaatcaatccgaaattcaaaatagaatattcaacttgacttaatcgcagttcattcgtgaatagacacatattagctctcgcgcgctttcttatcttcacagcttcttctcgatacctgaataagtacgggaccatttatgttcataagcattcagtgaaactgcagtctaaatactattggcatatacttat

SEQ ID NO: 346 - DNA, 2000 bp, Artificial Sequence
  Exemplary stuffer sequence
gatatgccatctatcgaggcctgttagcttaggacattacatgacagtgagacctagatatatagttgcatgagtagatgtaaccgaaggtactcagggacagaactgacggattgacgtttttcagtatcgtaaaagtttgagatccaacaatgaaagcttgatgcgccagatgatggaaatgcgcaaactgtcgtgtgataacacgggaattggtgctaagctggaatggtctaattcaagttccaatccatatccatctatgtgcgaggaatttgtaacggtaattatattgccttacaattattatcaaccaacacacttgaacgatgtaattgggggtatataccaataatagtactgccaactactgttttttgcaagaattaatcgtagtccgaattaaaagaaaagacggtgtacgcaacccaagtaattaaacgaataatcatacggtcgatatgctcattcgataaaacgcgagatctttaagttctctcaccggggtaatgcataattgccttaattggaaattgctttaggtgagagtcagtaaaccattggtgagatgtggttatactgcacctcacgcaaattaatattctaactttaacctgaattatgggttcccctcatcgggaagtatatctagtgccaacctatcacagttgcgcacatatgtttagaaatggttagtcggtcaggggaactcacgtaagcggtagtagtagaatttaatttatggtctcctaaagcatcgacatagtacactgcgaccattctaacacatactaaactttgaacttactgatatcttttatgtttgacttccttgctacgcaagtccaggcccagacagctgagttgtccttacacgagctatttgctgatcatatggtttaatcggcacgcgaattgcaagtttgatttaaggtgagcgcatacttgaatacagccagggagctccctactcagcgatcgtcttcagagatttcacgaaaatataagcattcccatcagaaattctaattaaaccttaccggaggtggggattactcgcagagttaaataatgagcccacattatgcgtttgcttctggagattatgggtggtttttcccgtaccgcctaatatagtatgcttcgactcagcaacttcactctaaaccctagagagcctctgtatgtacgcgcgtggatgaaatcaagaatggttggagtcaatgactggggcacaagtgtaatctggttcgattaatacatggcactaggtgctacgaggacgagtgaatgcaatatatgagtccttgctaataagcatcgaagatactctccggtactccttcatattcgactaatcggtgcactcaactttaggggggctccttattataaaatacatatagggtttgtttaaatgatttgttctattaatacgggcaaaattaatgcaatgttcacctaggcacgttggtactcgccgccaaacattggcattaatggggatacttagaaacaacataacatgaaaaatatctaggaacgccaacatatacgccgtgaccgtctgtcttaatagactctttttgtttaaagggtactgagtgattaactaatgctttccaatcctttccgttagaaggctattactacaagtgtttcccacgtgccgttaaaaatagaattatctttgtgggtttacgagcgcgtactgaaaacaggtttcttggatgggataatattatagatagcaataaagtaaactggaaaacagtattggatagcatgtgatggaccttgacccccttgtggcataagataatctcagcgtttcgttacacttacattcactgttaatgtctataggcaagttactatttggagtatttcaaagtgaacggaagaaatagaagtgctaacaaactccgtcatagtaggatcatatctccagagcgacctcatacatgctaaaaacctagtagacttcgtactatggattt

SEQ ID NO: 347 - DNA, 2000 bp, Artificial Sequence
  Exemplary stuffer sequence
aagacactttaccacataagtaaaccgttgacattatcgtggcggagagatactgcttgtactgggacactcagtattttgtggaatattgtacctagcgcctcgttccgtgaaagtgtggcatggattttcataattttatgctgtcctcattgcctacaattaatccagtaagcactagagaaatatctgctcctatgctgagattagccttatgaggtctttatatctttctgtaaaggccattgttcttttgatcctggagtctctgaattttgatttgtccctcaaagccttatgtgtacccggtcccggagcatgaagacgtatatcttgaagtaatccgaaagtatttaggtgtcgttgtccagtagtaatcccggttatgggttataattaagtgttaacatccgagcttggtctgtataatagtgtgtttgaatagtaaatatcaggactctacagggacctattctacttcgggttgtgtatcttccttggaataacttttgctacgcaaaaaagctataacaaggtctggagacggatgtgatttagtagggcaaatagatttaggtcttcgatagtacagaatactatgctacaaccaatctcttcatggctttatcaatacaatgttcttccttaactcagacgggagcaattatagttagctgaaggttgcctcacaatatgtgtcagagctagcgaaaagctcctaccaatatacatcagataaggagttcatacatctgtggccgatcaagcaagcaaggccgtccggttcacgacctgggtagtctgagtttggaggagaagccatcgcctctcgcattctactagagaaagatttcacacttactgacagagctacactggtacgacgaatctacaaaactaagcaaagtcctagggtgagcaatgcatggtaactagtacgattgatcagtgcgtggtatactatccggatagtccagacgtcaagacctaatcatcgtacgtaattaaataataatgcattcaactcttcggatacgatatatacttatatgcattaactatactttctcatgcattgtatctaacaaaatctgtacggcagaattaattactaaagtcttaatgattcgaatattaatatcaattttattacgaaacaaccaaactgacaacgtagagaggcaactacccagagtcgccaagaatactgtttacgaattgtagaaaagatgtaagaatgttcggatgtcggattacttaattgcgaacgtttgtcaagtcgttgcaggataccctcatctcctcttcctagtgaattatctgaaagtactattatacaatctaaatcggatacattcgtttgtaacaccacatggttggctcagctgaccatttacgcgcgatattctgtgctatccgaaggcgtaaaaggaattcaagtcagtctcctcttcgttatgtagaaagggaggactcctccgccgtatattcagctggctttaactaggaacatagttgcagttcaaacagtagaaaatcctggaagacatttcttgatagtctatctcagaaaaaggggggtgacgttcatgtttactaagacttgaaatgtggctccgtatctgcacaaccaggtttgggcggatgccggccgccatgtaacactgaacctcgcaagaaatgcacaattgaacaaatgaatactcacatcttatcgcttaatgttaaattcaaggcgagactggctcgaattattggagcctatgaagatgtatattaatgccaaggcaccgcacatagtaaagactatactaaccaagtgtgatattcaatcgatcgttgtggggaatcaggtacagttagtggcgaacagctttgacatccgtttaactttggcagcaccacaaaccctttgcgtacgtttttgtgttataaccaagttatgttgcaacctactttgacctcttatttctttgccgcaagactgaatgtcgtattat

SEQ ID NO: 348 - DNA, 2000 bp, Artificial Sequence
  Exemplary stuffer sequence
gagcaacctacggatatactatcgattctggacatggtaagtgtgttgcgtggttaataaaaagatttcgtggtcgggggtagatatacctgtaaggtttccaacagaccgctttgtagaaagagacttagtccctttgcaaaatgaggggaccgactaagaaagcgttgaattcaggtaatactttttgacgttaccatagttgttgcagtcccggagttaaacagagacacatcgtggcggagtccgtagtatcgcatgcgtggatttattgttgtaatcagatgttcaatatggcgtcaatatacaaataaacaggtcagatggagttagccttacttaaaaaacgaaaacaatgtatgccctaagcaaaaaaactagataaggacgatcaccacagttttaagagatctatatgcccctttgacatccttattctgacaatgggcagatccaactacaagatgtcgtaccgctaacacttgactaactaacgtcaagtaaaaagttcgttagtcatattatcaagtatggacttattcatcgacaggttgtaattagccctcccctagattagctgggctgaacccctattcctacgctcccttgtcacatgtattctctacctcaataggccggaaactcgcaagcccaagtatagcgtacggattaaattcgcgcaatcgctcttgaccatgttaaatgcttgcgcgtaacatcgaaaaggaggcaagacatttcagaagtaacatatcagttgacggcttacggtgctgaggtttaaaatccgactgattgctatcctatcgctgaggaatgactaaccttgcaaatccaagtctagaactgtcctagttctgtaccatgcccagcgttcggatgtcagtacgtgtatgcagcatttaggaggtgatgtctcccagtcggtcaataagctttgcttacctcacggataactaagttcatctccagtgtacgaagattctctagcactaactattcattgtaactaattggtatccgactttaagccatagtgtggcatgacgtaagttatgtcagttctttggaactttttgcgcagctgtgttgacgaaacacaggttgcaggttggtctaggtaagggatgcactcactgcgatgtgatcctttaatggccatttaaatctatctcgagtatagcgtgtatacttactatgaagcaaattagtatacatataacaatgaatatacacatagtgggaggttgccattcatccatgtaggcatgtaatatggcacctcctctttggatacagaggcccatgcctccgaatcacatatttacttaaacagttaacggaattcaggtatcccgtttcattattcgaaacgtctctggggttaccttacttacgttatctgcatgagaatagagtccatcggcgtttctaacaatcaatcatgcttgcaattcagcgagtgtagaggaattgtaagaacgccggatgctccctttaccttatccgcacaggcccctacgattgaactattgaaagttttattacaaatctcatatatgggggagcagttaaagttctgcataagaaggacctaggataatgccataaaaggttgatatggaaatactattggaataagaaagtatatggtgtctataatggatatatcagtaaacgaaggcatttcttacactttgatttcattaactgtaatctctatttgtgttggcgaatccggtaaacagaggtttataactggtttaccttagtcgagtgtcttagatatacatgtcgattcagatcaatcctactcatcccaaacgcacatgtcacgatacgtactttatacagtaagaggcacaatgtgggtgccctctctcgtccgacttattgcggacggagaaatagttagtacggactgtcacaagtctgtaaccactaaagatcgggcagctcagacattattgaaggtaggccaaagtatcattaatgctttg

SEQ ID NO: 349 - DNA, 2000 bp, Artificial Sequence
  Exemplary stuffer sequence
attaataaatgtctaacggtctagaaatgcacctaatttgctactgctgaactcctgattactcctcctcgtttatacttgttcattaagaattttttccgtctagattaagtacacggtaatacacacgattaaatacaccgccacagatcttcgctatcaatattacattttgttcactcattacgataagcgtggcttggctgagttctagacttatcgtgttaacgtcaatgaaaacttatggatttgaagctacgatgctaatctaactttaccttaagcaagaaagaccttcgttaataggacccttaaagcctgtgatgtcggttaaacggttctagtttgatagtgacgttagggactcggtatacatcttagccgaactgtctaaattactttagagaaacttttccctgggggaggcacgttccgtttatggacctcatttgagactcaatatgtacaactaatagtgtgattagatcctgattcccatacgtatcggctcgcccttaatcaatacagatccgtgctatgtccatactgcgattccaaaggttgtctaacaagacaaacttgagagaggcttcacaaagcaacccagcacccttgtcctcttttttaggggtacgctgacatctggatgcattaagaaatacgtatctagaaggatcgcgataagtcgcacaagtttaccaccttatattctgcaggctgctattggaggtaatacgtgctcgcacacgcccaagtgaggcattcttacaagacttaccttacagcctattaataacgtcgaattttgcgcagcaaccaattccagggcaaactataagccttattgaggttaatagggcgcaatatatttacgatagaaggtaaatctataatactgtcacttgtcaatgatgatggtctaactaattgattcccatgcaagtggcgaaccaggcttactttagtttaatagcgatcaagtatactaagcacacactgaatgtatcacataagatacgtaaaataaatcaactcattaaatcaaagacagattcacaaatgtttcgtgttttaacagatctgaatataaactctgctgatgtgatcgtaggacgtaagaaggtatagttgaagaatagcgtgaatatctgatctctgttagcaaatacatcacgattatcaccaggtttaccacaacaataagattgtgactgacactactttctatatgaatgtattctcatgaggatgcgtaagacgtataggatcatactgaattataactccatattagggtctatatcacatacatctccaagttaaaaagtctattggcgattccacacaactcgcgctagtagtacattttaccggtaccggtacagtctaagttattgatctaggttcaacttctaaaatactgaagtctcaggtatatagaatttatactactcgcgggacgtaaagcccctctgtggttagcgtcgcagcgtcgagtaaattccttatagagcctaaaccttgataatttcgacgtaccgttataacgcaattaatagacttctcattttcctgccgagtcgggtctggtatagtctaggacgggggtagatatgatcgtcgtcttctctaatctaatttaatctataaccacagcgtacaagtaaggtatgtaagatacagagataaattagagatttgtgttactccgcatgttgaactaaacccaaaggttcacgccgtatgcctttcaagttcctccgctcaaaaggctccgggtgtcccctacccgatatggcggaaatcgttaattctcataacgaccaaccttaccttggacacacctaagcactaagtcggtaaatggagtacacaatgtgggagttgtgtttaacataatgaggctcgttcagactatgttcgaggcgtataacgatttgtgacagattcctcatcaactcgggtcagatttatagcaatggtaaattccctatatccta

SEQ ID NO: 350 - DNA, 2000 bp, Artificial Sequence
  Exemplary stuffer sequence
tatggtgtggcacatatgaataaaacaaggagaagcagccgacaatacttagaacgtgtcagaacaatcaagatgtctgaaacgttcaacaatcgagttattccgggctaatttattcccatccttatatacagagccgcacaataccaagtaacgtgctttgggccacgaactcactctagtcttccggaccctccggtactactcggtatggtggatattcatgagaatggttttagtcttaaaaaaatgtgaacaagaaaacatttacgtccaagaaagcggtattttgtttgggtctaggaaacaatcagtcgtggacctgggcgagatcggctgttttcgaccgattttatgctaagcagaaggaagtgaccgaggttgtgtttagatccagtaaaagtcgtcatacccgaggagatttctgtggtgcctagtgactagcgatcccgtgcagcagttcaaatgcgctggatagttcgctcctgcaccactagttcacaccagaagtatgtcttttaagagactgtctaagaaatatagtctctaaacgtgactatcgttcactccctgtacaaatctaggactaacgggtatagattaaacgtattagaatttcggagcattagaattttgttgttctaagttaggatgatttcaagtgtccatgtaaattgaggtcaatataggacgatctacatccgagataggccaagtacgattctgtgttacattttgcgttcgcacaagctaggacgagggtatgagcattttgtgctaaccgaatgagatgcagcttattgtatccttacccgcaacatagggcatgaaggcgtggttcgagaatcgcgcgagataaatacatgtttcgatttatgtcaaccactgcaatggtttataaatgttattcaagcatcgattcaataacctctggatgtagtaatatctgcgggtgtgtaagtgcgatatcctaagtcgggagatttaacaataccttgggatgctccggacaattttcgacgtacgcaattatgaacatgcattgattgactaaacttaagaaacataatcagtgtatagtattgtaacaatggattctgagtgtctaatgttttctcgctccatgttataacacataattatacttataataccatcccatctttaagtacaaaaccttgttgcgctgctttatggagactattgagcccaacgggttgagtggttattactatttgaagtaaaagcagtatctactcagattcctagaggtaaatatgaacttgttttctatctggttatctatttttagttttatggatatggacgaagttaaaagttatagacctgacattcttctcccataggtatagaagtggagttaaacaagttcttagtgggggaaatgacgtacagactactatcttgatgatagcttttcgatcaaacaagagtttcaaccgctgtaaaggtttatatgcgatgtagtgtggtacgataacgtactttgccgatcattcactgattccattaggtacgacactctcagttacaaagcggtactaacctagcaaaaagtgaatatcgccctacaaactattactggagtgcggtggcagctttggcgaaaattggccgaactctttgctgtttatatggtaactattctcactatgctactgattggaaaaagatatttgccaactaatagtcgtaatgttagtattgatagggataataggcatttaaagttccctgaaacatacggtaaataagatctcttttaacaacaccaggggtggctcactggggtagcaaatacttaacgatccctttttcatcaagtgagttatctgctttggattcttacaactagatgttataaagaaagaagctgcgcagtttgcatgactaaaatttatatgaagtagtagttattagtactatctcttagtaggctagaatgtaaacctgcagacatcatggaatgcacatacccg

SEQ ID NO: 351 - DNA, 2000 bp, Artificial Sequence; identical to SEQ ID NO: 251
  Exemplary stuffer sequence

SEQ ID NO: 352 - DNA, 2000 bp, Artificial Sequence; identical to SEQ ID NO: 261
  Master stuffer

SEQ ID NO: 353 - DNA, 2534 bp, Artificial Sequence
  Donor template design 1
ttatccccttcctatgacatgaacttaaccatagaaaagaaggggaaagaaaacatcaagcgtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagctctcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttggaatcacagcttggtaagcatattgaagatcgttttcccaattttcttattacacaaataagaagttgatgcactaaaagtggaagagttttgtctaccataattcagctttgggatatgtagatggatctcttcctgcgtctccagaatatgc

SEQ ID NO: 354 - DNA, 2383 bp, Artificial Sequence
  Donor template design 2
gtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctctactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacggtctaagtgatgacagccgtaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagctccaaaggactcaaagaacctctggatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagatactcaggagtcagatgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtga

SEQ ID NO: 355 - DNA, 2673 bp, Artificial Sequence
  Donor template design 3
tactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagataaccgcctatgcggccatgccaggattatagataactttttaacattagccgcagaggtgggactagcacgtaatatcagcacataacgtgtcagtcaggtcatcgacctcgtcggactccgggtgcgaggtcgtgaagctggaatacgagtgaggccgccgaggacgtcaggggggtgtaaagcttccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgacgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaagcttgcgacctggaatcggacagcagcggggagtgtacggccccgagttcgtgaccgggtatgctttcattgtaccccggaactttaaatctatgaacaatcgcaacaaattgtccaaaggcaacaatacgacacagttagaggccatcggcgcaggtacactctatccacgcctatcagaatgtcacctggttaatggtcaatttaggtggctggaggcacatgtgaagcaatatggtctagggaaagatatcggtttacttagattttatagttccggatccaacttaaataatataggtattaaagagcagtatcaagagggtttcttcccaaggaatcttgcgattttcatacacagctttaacaaatttcactagacgcaccttcattttgtcgtctcgttgtatatgagtccggggtaagaattttttaccgtatttaacatgatcaacgggtactaaagcaatgtcatttctaaacacagtaggtaaaggacacgtcatcttattttaaagaatgtcagaaatcagggagactagatcgatattacgtgtttt